(19) United States
(12) Patent Application Publication
(10) Pub. No.: US 2024/0358820 A1
(43) Pub. Date: Oct. 31, 2024

(54) PROTOPARVOVIRUS COMPOSITIONS COMPRISING A PROTOPARVOVIRUS VARIANT VP1 CAPSID POLYPEPTIDE AND RELATED METHODS

(71) Applicant: Carbon Biosciences, Inc., Waltham, MA (US)

(72) Inventors: Robert Kotin, Cambridge, MA (US); Sebastian Aguirre Kozlouski, Cambridge, MA (US); Carolyn Pelletier, Salem, MA (US)

(21) Appl. No.: 18/614,158

(22) Filed: Mar. 22, 2024

Related U.S. Application Data
(60) Provisional application No. 63/545,449, filed on Oct. 24, 2023.
(60) Provisional application No. 63/454,259, filed on Mar. 23, 2023.

(51) Int. Cl. A61K 39/23; A61K 39/00

(52) U.S. Cl. CPC A61K 39/23; A61K 2039/5256; C12N 2750/14322; C12N 2750/14334; C12N 2750/14362

(57) ABSTRACT

The present disclosure provides technologies comprising compositions, preparations, constructs, and methods comprising a protoparvovirus variant VP1 capsid polypeptide.

### CROSS REFERENCE TO RELATED APPLICATIONS

This application claims the benefit of U.S. Application Ser. No. 63/454,259 filed on Mar. 23, 2023, and U.S. Application Ser. No. 63/545,449 filed on Oct. 24, 2023, the disclosures of each of which are hereby incorporated by reference in their entireties.

### SEQUENCE LISTING

This application contains a Sequence Listing, which has been submitted electronically through USPTO Patent Center in XML format and is hereby incorporated by reference in its entirety. Said XML copy, created on May 1, 2024, is named “2017359-0074.xml” and is 308,234 bytes in size.

### BACKGROUND

Viral particles (or virions) are commonly utilized for gene therapy. The present disclosure provides technologies relating to protoparvovirus variant VP1 capsid polypeptides, their production and use, including in gene therapy.

### SUMMARY

The present disclosure recognizes a need for improvements in gene therapy technologies. For example, among other things, the present disclosure recognizes a need for improved compositions, preparations, constructs, virions, populations of virions, host cells, etc. Furthermore, the present disclosure specifically recognizes a need for improved production and manufacturing of virions that comprise or otherwise utilize a protoparvovirus VP1 capsid polypeptide.

Among other things, the present disclosure provides an insight that improving retention of a protoparvovirus VP1 capsid polypeptide in cytoplasm of a cell can provide a variety of benefits. Alternatively or additionally, the present disclosure recognizes a need for reduced toxicity of virions comprising a protoparvovirus VP1 capsid polypeptide in cytoplasm of a cell. For example, in some embodiments, retention of a protoparvovirus VP1 capsid polypeptide can lead to cell toxicity, thereby reducing protoparvovirus VP1 capsid polypeptide yield.

Among other things, in some embodiments, the present disclosure recognizes that one or more characteristic sequence elements of a protoparvovirus VP1 capsid polypeptide surprisingly affects internalization of virions into a host cell. Among other things, in some embodiments, the present disclosure recognizes that one or more characteristic sequence elements of a protoparvovirus VP1 capsid polypeptide surprisingly affects virion transit into a nucleus of a cell. Among other things, the present disclosure recognizes that one or more characteristic sequence elements of a protoparvovirus VP1 capsid polypeptide surprisingly affects protoparvovirus VP1 capsid polypeptide expression in a host cell. Among other things, the present disclosure recognizes that one or more characteristic sequence elements of a protoparvovirus VP1 capsid polypeptide surprisingly affects protoparvovirus VP1 capsid polypeptide toxicity in a host cell.

In some embodiments, a characteristic sequence element comprises one or more stretches of amino acid residues within a protoparvovirus VP1 capsid polypeptide. In some embodiments, a characteristic sequence element comprises one or more stretches of amino acid residues within a protoparvovirus VP1 unique region (VP1u). In some embodiments, a characteristic sequence element comprises a protoparvovirus nuclear localization signal sequence (NLS) within a protoparvovirus VP1 capsid polypeptide. In some embodiments, a characteristic sequence element comprises a phospholipase A2 (PLA2) motif within a protoparvovirus VP1 capsid polypeptide. In some embodiments, a characteristic sequence element comprises a stretch of amino acid residues between a NLS and a PLA2 motif within a protoparvovirus VP1 capsid polypeptide. In some embodiments, a characteristic sequence element between a NLS and a PLA2 motif within a protoparvovirus VP1 capsid polypeptide comprises at least one sequence variation that improves characteristic features of compositions, preparations, constructs, virions, population of virions, and host cells for gene therapy and related methods described herein, relative to a protoparvovirus reference VP1 capsid polypeptide. In some embodiments, at least one sequence variation comprises one or more deletions of a stretch of amino acid residues between a NLS and a PLA2 motif of a protoparvovirus VP1capsid polypeptide as described herein.

For example, in some embodiments, the present disclosure recognizes a splicing event that occurs in a protoparvovirus VP1 capsid polypeptide which eliminates a characteristic sequence element between a NLS and a PLA2 motif within a protoparvovirus VP1 capsid polypeptide. Surprisingly, it is an insight of the present disclosure that such splicing event is not guaranteed to occur during infection and/or production of a virion in a host cell. Moreover, surprisingly, it is an insight of the present disclosure that such splicing event is dependent on a type of host cell that is being infected and/or used to produce a virion.

Therefore, in some embodiments, the present disclosure describes that deletion of one or more amino acid residues of a characteristic sequence element between a NLS and a PLA2 motif within a protoparvovirus VP1 capsid polypeptide resulted in a significant increase of expression of a protoparvovirus variant VP1 capsid polypeptide in a host cell, relative to a protoparvovirus reference VP1 capsid polypeptide. In some embodiments, deletion of five amino acid residues between a NLS and a PLA2 motif within a protoparvovirus VP1 capsid polypeptide resulted in significant reduced toxicity of a protoparvovirus variant VP1 capsid polypeptide in a host cell, relative to a protoparvovirus reference VP1 capsid polypeptide. In some embodiments, deletion of five amino acid residues between a NLS and a PLA2 motif within a protoparvovirus VP1 capsid polypeptide resulted in significant improvement of VP1 capsid polypeptide expression, relative to a protoparvovirus reference VP1 capsid polypeptide in a host cell.

Among other things, is an insight of the present disclosure that a VP1 capsid coding sequence encoding a protoparvovirus reference VP1 capsid polypeptide may comprise an unwanted out-of-frame ATG which can affect protoparvovirus VP1 capsid polypeptide expression and/or formation. Among other things, in some embodiments, constructs described herein comprise one or more nucleotide modifications to remove out-of-frame ATG in a protoparvovirus VP1 capsid polypeptide (e.g., a protoparvovirus VP1u capsid polypeptide).

Among other things, in some embodiments, the present disclosure provides compositions, preparations, constructs, virions, population of virions, and host cells comprising a protoparvovirus variant VP1 capsid polypeptide for gene therapy. In some embodiments, a protoparvovirus variant VP1 capsid polypeptide is characterized by reduced toxicity in a host cell, relative to a protoparvovirus reference VP1 capsid polypeptide. In some embodiments, a protoparvovirus variant VP1 capsid polypeptide is characterized by improved production of a protoparvovirus variant VP1 capsid polypeptide in a host cell, relative to a protoparvovirus reference VP1 capsid polypeptide. In some embodiments, a protoparvovirus variant VP1 capsid polypeptide is characterized by increased retention of a protoparvovirus variant VP1 capsid polypeptide in a host cell, relative to a protoparvovirus reference VP1 capsid polypeptide. In some embodiments, a host cell is an insect cell. In some embodiments, a protoparvovirus variant VP1 capsid polypeptide is characterized by increased expression of a protoparvovirus variant VP1 capsid polypeptide in a host cell, relative to a protoparvovirus reference VP1 capsid polypeptide. In some embodiments, deletion of five amino acid residues between a NLS and a PLA2 motif within a protoparvovirus VP1 resulted in significant improvement of increased capsid polypeptide yield, relative to a protoparvovirus reference VP1 capsid polypeptide. In some embodiments, an insect cell is a Sf9 cell. In some embodiments, a host cell is a mammalian cell.

Among other things, in some embodiments, the present disclosure provides a construct comprising a VP1 capsid coding sequence operably linked to an expression control sequence, wherein the VP1 capsid coding sequence encodes a protoparvovirus variant VP1 capsid polypeptide wherein the protoparvovirus variant VP1 capsid polypeptide comprises at least one sequence variation relative to the protoparvovirus reference VP1 capsid polypeptide. In some embodiments, a protoparvovirus variant VP1 capsid polypeptide comprises a deletion of one or more amino acid residues downstream of a NLS sequence. In some embodiments, an expression control sequence is a promoter that improves protoparvovirus variant VP1 capsid polypeptide initiation. In some embodiments, a construct comprises a 5′ untranslated region (UTR). In some embodiments, a 5′ UTR sequence improves protoparvovirus variant VP1 capsid polypeptide initiation. For example, in some embodiments, a 5′ UTR sequence comprises a nucleotide spacer sequence. In some embodiments, a 5′ UTR sequence comprises a nucleotide spacer sequence that does not comprise an alternative translation initiation sequence (e.g., ATT, ATA, ATC). In some embodiments, a 5′ UTR sequence comprises a Kozak consensus sequence, or portion thereof. In some embodiments, such portion of a Kozak consensus sequence comprises a single nucleotide. In some embodiments, such portion of a Kozak consensus sequence comprises one to three nucleotides. In some embodiments, such portion of a Kozak consensus sequence comprises one to five nucleotides. In some embodiments, a 5′ UTR sequence comprises a nucleotide spacer sequence and a Kozak consensus sequence. In some embodiments, a 5′ UTR sequence does not comprise a nucleotide spacer sequence. In some embodiments, at least one Kozak residue may be within a translated region of a construct described herein. In some embodiments, a Kozak residue may be within a translated region of a construct described herein. In some embodiments, a 5′ UTR sequence comprises a stretch of nucleotides between an expression control sequence and a VP1 capsid coding sequence. In some embodiments, a Kozak consensus sequence comprises a eukaryotic sequence (GCCGCC - - - G). In some embodiments, a Kozak consensus sequence comprises a viral-derived Kozak consensus sequence (CCTGTTAAG). In some embodiments, a Kozak consensus sequence comprises an alternative Kozak consensus sequence (AAA). In some embodiments a construct comprises a VP1 translation initiation codon sequence of CTG. In some embodiments a construct comprises a VP1 translation initiation codon sequence of TTG. In some embodiments a construct comprises a VP1 translation initiation codon sequence of ACG. In some embodiments a construct comprises a VP1 translation initiation codon sequence of ATC. In some embodiments a construct comprises a VP1 translation initiation codon sequence of ATG.

Moreover, among other things, in some embodiments, the present disclosure provides that protoparvovirus is not as prevalent as AAV. Thus, among other things, administration (e.g., systemic administration) of compositions (e.g., pharmaceutical compositions), preparations, constructs, virions, population of virions comprising a protoparvovirus VP1 capsid polypeptide to a subject would not trigger an extensive anti-viral immune reaction that precludes efficient gene delivery. Accordingly, in some embodiments, prescreening a subject for anti-protoparvovirus antibodies is not required prior to administering (e.g., systemically) compositions (e.g., pharmaceutical compositions), preparations, constructs, virions, population of virions described herein.

Moreover, among other things, in some embodiments, the present disclosure describes that the provided compositions (e.g., pharmaceutical compositions), preparations, constructs, virions, population of virions can be administered (e.g., systemically) to a subject to achieve expression of a heterologous nucleic acid (or payload) in specific target cells, tissues, and/or organs as described herein. Importantly, unlike AAV for example, the provided compositions (e.g., pharmaceutical compositions), preparations, constructs, virions, population of virions can be administered (e.g., systemically) to a subject to achieve expression of a heterologous nucleic acid (or payload) in specific target cells, tissues, and/or organs as described herein, with minimal targeting to liver cells.

In some embodiments, provided compositions, preparations, constructs, virions, population of virions, and host cells are for use in methods of treatment, delivery, producing polypeptides, or delaying/arresting progression of a disease or disorder.

In some embodiments, provided compositions, preparations, constructs, virions, population of virions, and host cells are for use in methods of manufacturing.

In some embodiments, provided compositions, preparations, constructs, virions, population of virions, and host cells are for use in methods of characterization.

In some embodiments, provided compositions, preparations, constructs, virions, population of virions, and host cells are for use in methods of purification.

Elements of embodiments involving one aspect of the invention (e.g., systems) can be applied in embodiments involving other aspects of the invention, and vice versa.

Elements of embodiments involving one aspect of the invention (e.g., methods) can be applied in embodiments involving other aspects of the invention, and vice versa.

### Definitions

The scope of the present disclosure is defined by the claims appended hereto and is not limited by certain embodiments described herein. Those skilled in the art, reading the present specification, will be aware of various modifications that may be equivalent to such described embodiments, or otherwise within the scope of the claims. In general, terms used herein are in accordance with their understood meaning in the art, unless clearly indicated otherwise. Explicit definitions of certain terms are provided below; meanings of these and other terms in particular instances throughout this specification will be clear to those skilled in the art from context.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The articles “a” and “an,” as used herein, should be understood to include plural referents unless clearly indicated to the contrary. Claims or descriptions that include “or” between one or more members of a group are considered satisfied if one, more than one, or all of the group members are present in, employed in, or otherwise relevant to a given product or process unless indicated to the contrary or otherwise evident from the context. In some embodiments, exactly one member of a group is present in, employed in, or otherwise relevant to a given product or process. In some embodiments, more than one, or all group members are present in, employed in, or otherwise relevant to a given product or process. It is to be understood that the present disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists (e.g., in Markush group or similar format), it is to be understood that each subgroup of the elements is also disclosed, and any element(s) can be removed from the group. It should be understood that, in general, where embodiments or aspects are referred to as “comprising” particular elements, features, etc., certain embodiments or aspects “consist,” or “consist essentially of,” such elements, features, etc. For purposes of simplicity, those embodiments have not in every case been specifically set forth in so many words herein. It should also be understood that any embodiment or aspect can be explicitly excluded from the claims, regardless of whether the specific exclusion is recited in the specification.

Throughout the specification, whenever a polynucleotide or polypeptide is represented by a sequence of letters (e.g., A, C, G, and T, which denote adenosine, cytidine, guanosine, and thymidine, respectively, in the case of a polynucleotide), such polynucleotides or polypeptides are presented in 5′ to 3′ or N-terminus to C-terminus order, from left to right.

Administration: As used herein, the term “administration” typically refers to administration of a composition to a subject or system to achieve delivery of an agent to a subject or system. In some embodiments, an agent is, or is included in, a composition; in some embodiments, an agent is generated through metabolism of a composition or one or more components thereof. Those of ordinary skill in the art will be aware of a variety of routes that may, in appropriate circumstances, be utilized for administration to a subject, for example a human. For example, in some embodiments, administration may be systematic or local. In some embodiments, a systematic administration can be intravenous. In some embodiments, administration can be local. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Amelioration: As used herein, the term “amelioration” refers to prevention, reduction or palliation of a state, or improvement of a state of a subject. Amelioration may include, but does not require, complete recovery or complete prevention of a disease, disorder or condition.

Amino acid: In its broadest sense, as used herein, the term “amino acid” refers to any compound and/or substance that can be incorporated into a polypeptide chain, e.g., through formation of one or more peptide bonds. In some embodiments, an amino acid has a general structure, e.g., H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than standard amino acids, regardless of whether it is prepared synthetically or obtained from a natural source. In some embodiments, an amino acid, including a carboxy- and/or amino-terminal amino acid in a polypeptide can contain a structural modification as compared with general structure as shown above. For example, in some embodiments, an amino acid may be modified by methylation, amidation, acetylation, pegylation, glycosylation, phosphorylation, and/or substitution (e.g., of an amino group, a carboxylic acid group, one or more protons, and/or a hydroxyl group) as compared with a general structure. In some embodiments, such modification may, for example, alter circulating half-life of a polypeptide containing a modified amino acid as compared with one containing an otherwise identical unmodified amino acid. In some embodiments, such modification does not significantly alter a relevant activity of a polypeptide containing a modified amino acid, as compared with one containing an otherwise identical unmodified amino acid.

Approximately or About: As used herein, the terms “approximately” or “about” may be applied to one or more values of interest, including a value that is similar to a stated reference value. In some embodiments, the term “approximately” or “about” refers to a range of values that fall within +10% (greater than or less than) of a stated reference value unless otherwise stated or otherwise evident from context (except where such number would exceed 100% of a possible value). For example, in some embodiments, the term “approximately” or “about” may encompass a range of values that within 10%, 9%, 8%, 7%, 6%, 5%, 4%, 3%, 2%, 1%, or less of a reference value.

Associated: As used herein, the term “associated” describes two events or entities as “associated” with one another, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biologically active: As used herein, the term “biologically active” refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest.

Characteristic portion: As used herein, the term “characteristic portion,” in the broadest sense, refers to a portion of a substance whose presence (or absence) correlates with presence (or absence) of a particular feature, attribute, or activity of the substance. In some embodiments, a characteristic portion of a substance is a portion that is found in a given substance and in related substances that share a particular feature, attribute or activity, but not in those that do not share the particular feature, attribute or activity. In some embodiments, a characteristic portion shares at least one functional characteristic with the intact substance. For example, in some embodiments, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to a sequence and/or structural identity specified above, shares at least one functional characteristic with the relevant intact substance. In some embodiments, a characteristic portion may be biologically active.

Characteristic sequence: As used herein, the term “characteristic sequence” is a sequence that is found in all members of a family of polypeptides or nucleic acids, and therefore can be used by those of ordinary skill in the art to define members of the family.

Characteristic sequence element: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of a polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share a sequence element.

Cleavage: As used herein, the term “cleavage” refers to generation of a break in DNA. For example, in some embodiments, cleavage could refer to either a single-stranded break or a double-stranded break depending on a type of nuclease that may be employed to cause such a break.

Combination therapy: As used herein, the term “combination therapy” refers to those situations in which a subject is simultaneously exposed to two or more therapeutic regimens (e.g., two or more therapeutic agents). In some embodiments, two or more agents may be administered simultaneously. In some embodiments, two or more agents may be administered sequentially. In some embodiments, two or more agents may be administered in overlapping dosing regimens.

Comparable: As used herein, the term “comparable” refers to two or more agents, entities, situations, sets of conditions, subjects, populations, etc., that may not be identical to one another but that are sufficiently similar to permit comparison therebetween so that one skilled in the art will appreciate that conclusions may reasonably be drawn based on differences or similarities observed. In some embodiments, comparable sets of agents, entities, situations, sets of conditions, subjects, populations, etc. are characterized by a plurality of substantially identical features and one or a small number of varied features. Those of ordinary skill in the art will understand, in context, what degree of identity is required in any given circumstance for two or more such agents, entities, situations, sets of conditions, subjects, populations, etc. to be considered comparable. For example, those of ordinary skill in the art will appreciate that sets of agents, entities, situations, sets of conditions, subjects, populations, etc. are comparable to one another when characterized by a sufficient number and type of substantially identical features to warrant a reasonable conclusion that differences in results obtained or phenomena observed under or with different sets of circumstances, stimuli, agents, entities, situations, sets of conditions, subjects, populations, etc. are caused by or indicative of the variation in those features that are varied.

Construct: As used herein, the term “construct” refers to a composition including a polynucleotide capable of carrying at least one heterologous polynucleotide. In some embodiments, a construct can be a plasmid, a transposon, a cosmid, an artificial chromosome (e.g., a human artificial chromosome (HAC), a yeast artificial chromosome (YAC), a bacterial artificial chromosome (BAC), or a P1-derived artificial chromosome (PAC)) or a viral construct, and any Gateway® plasmids. A construct can, e.g., include sufficient cis-acting elements for expression; other elements for expression can be supplied by the host primate cell or in an in vitro expression system. A construct may include any genetic element (e.g., a plasmid, a transposon, a cosmid, an artificial chromosome, or a viral construct, etc.) that is capable of replicating when associated with proper control elements. Thus, in some embodiments, “construct” may include a cloning and/or expression construct and/or a viral construct (e.g., an adeno-associated virus (AAV) construct, an adenovirus construct, a lentivirus construct, or a retrovirus construct).

Conservative: As used herein, the term “conservative” refers to instances describing a conservative amino acid substitution, including a substitution of an amino acid residue by another amino acid residue having a side chain R group with similar chemical properties (e.g., charge or hydrophobicity). In general, a conservative amino acid substitution will not substantially change functional properties of interest of a protein, for example, ability of a receptor to bind to a ligand. Examples of groups of amino acids that have side chains with similar chemical properties include: aliphatic side chains such as glycine (Gly, G), alanine (Ala, A), valine (Val, V), leucine (Leu, L), and isoleucine (Ile, I); aliphatic-hydroxyl side chains such as serine (Ser, S) and threonine (Thr, T); amide-containing side chains such as asparagine (Asn, N) and glutamine (Gln, Q); aromatic side chains such as phenylalanine (Phe, F), tyrosine (Tyr, Y), and tryptophan (Trp, W); basic side chains such as lysine (Lys, K), arginine (Arg, R), and histidine (His, H); acidic side chains such as aspartic acid (Asp, D) and glutamic acid (Glu, E); and sulfur-containing side chains such as cysteine (Cys, C) and methionine (Met, M). Conservative amino acids substitution groups include, for example, valine/leucine/isoleucine (Val/Leu/Ile, V/L/I), phenylalanine/tyrosine (Phe/Tyr, F/Y), lysine/arginine (Lys/Arg, K/R), alanine/valine (Ala/Val, A/V), glutamate/aspartate (Glu/Asp, E/D), and asparagine/glutamine (Asn/Gln, N/Q). In some embodiments, a conservative amino acid substitution can be a substitution of any native residue in a protein with alanine, as used in, for example, alanine scanning mutagenesis. In some embodiments, a conservative substitution is made that has a positive value in the PAM250 log-likelihood matrix disclosed in Gonnet et al., 1992, Science 256:1443-1445, which is incorporated herein by reference in its entirety. In some embodiments, a substitution is a moderately conservative substitution wherein the substitution has a nonnegative value in the PAM250 log-likelihood matrix. One skilled in the art would appreciate that a change (e.g., substitution, addition, deletion, etc.) of amino acids that are not conserved between the same protein from different species is less likely to have an effect on the function of a protein and therefore, these amino acids should be selected for mutation. Amino acids that are conserved between the same protein from different species should not be changed (e.g., deleted, added, substituted, etc.), as these mutations are more likely to result in a change in function of a protein.

CONSERVATIVE AMINO ACID SUBSTITUTIONS

| For Amino Acid | Code | Replace With |
|---|---|---|
| Alanine | A | D-ala, Gly, Aib, β-Ala, Acp, L-Cys, D-Cys |
| Arginine | R | D-Arg, Lys, D-Lys, homo-Arg, D-homo-Arg, Met, Ile, |
| | | D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, Aib, B-Ala, Acp |
| Isoleucine | I | D-Ile, Val, D-Val, AdaA, AdaG, Leu, D-Leu, Met, D- |
| | | Met |
| Leucine | L | D-Leu, Val, D-Val, AdaA, AdaG, Leu, D-Leu, Met, D- |
| | | Met |
| Lysine | K | D-Lys, Arg, D-Arg, homo-Arg, D-homo-Arg, Met, D- |
| | | Met, Ile, D-Ile, Orn, D-Orn |
| Methionine | M | D-Met, S-Me-Cys, Ile, D-Ile, Leu, D-Leu, Val, D-Val |
| Phenylalanine | F | D-Phe, Tyr, D-Thr, L-Dopa, His, D-His, Trp, D-Trp, |
| | | Trans-3,4 or 5-phenylproline, AdaA, AdaG, cis-3,4 or |
| | | 5-phenylproline, Bpa, D-Bpa |
| Proline | P | D-Pro, L-I-thioazolidine-4-carboxylic acid, D-or-L-1- |
| | | oxazolidine-4-carboxylic acid (Kauer, U.S. Pat. No. |
| | | 4,511,390) |
| Serine | S | D-Ser, Thr, D-Thr, allo-Thr, Met, D-Met, Met (O), D- |
| | | Met (O), L-Cys, D-Cys |
| Threonine | T | D-Thr, Ser, D-Ser, allo-Thr, Met, D-Met, Met (O), D- |
| | | Met (O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met, AdaA, |
| | | AdaG |

Control: As used herein, the term “control” refers to the art-understood meaning of a “control” being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. For example, in one experiment, a “test” (i.e., a variable being tested) is applied. In a second experiment, a “control,” the variable being tested is not applied. In some embodiments, a control is a historical control (e.g., of a test or assay performed previously, or an amount or result that is previously known). In some embodiments, a control is or comprises a printed or otherwise saved record. In some embodiments, a control is a positive control. In some embodiments, a control is a negative control.

Determining, measuring, evaluating, assessing, assaying and analyzing: As used herein, the terms “determining,” “measuring,” “evaluating,” “assessing,” “assaying,” and “analyzing” may be used interchangeably to refer to any form of measurement, and include determining if an element is present or not. These terms include both quantitative and/or qualitative determinations. Assaying may be relative or absolute. For example, in some embodiments, “Assaying for the presence of” can be determining an amount of something present and/or determining whether or not it is present or absent.

Editing: As used herein, the term “edit,” “editing,” or “edited” refers to a method of altering a nucleic acid sequence of a polynucleotide (e.g., a wild type naturally occurring nucleic acid sequence or a mutated naturally occurring sequence) by selective deletion of a specific nucleic acid sequence (e.g., a genomic target sequence), a given specific inclusion of new sequence through use of an exogenous nucleic acid sequence, or a replacement of nucleic acid sequence with an exogenous nucleic acid sequence. In some embodiments, such a specific genomic target includes, but may be not limited to, a chromosomal region, mitochondrial DNA, a gene, a promoter, an open reading frame or any nucleic acid sequence.

Engineered: In general, as used herein, the term “engineered” refers to an aspect of having been manipulated by the hand of man. For example, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Excipient: As used herein, the term “excipient” refers to an inactive (e.g., non-therapeutic) agent that may be included in a pharmaceutical composition, for example to provide or contribute to a desired consistency or stabilizing effect. In some embodiments, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: As used herein, the term “expression” of a nucleic acid sequence refers to generation of any gene product (e.g., transcript, e.g., mRNA, e.g., polypeptide, etc.) from a nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Functional: As used herein, the term “functional” describes something that exists in a form in which it exhibits a property and/or activity by which it is characterized. For example, in some embodiments, a “functional” biological molecule is a biological molecule in a form in which it exhibits a property and/or activity by which it is characterized. In some such embodiments, a functional biological molecule is characterized relative to another biological molecule which is non-functional in that the “non-functional” version does not exhibit the same or equivalent property and/or activity as the “functional” molecule. A biological molecule may have one function, two functions (i.e., bifunctional) or many functions (i.e., multifunctional).

Gene: As used herein, the term “gene” refers to a DNA sequence in a chromosome that codes for a gene product (e.g., an RNA product, e.g., a polypeptide product). In some embodiments, a gene includes coding sequence (i.e., sequence that encodes a particular product). In some embodiments, a gene includes non-coding sequence. In some particular embodiments, a gene may include both coding (e.g., exonic) and non-coding (e.g., intronic) sequence. In some embodiments, a gene may include one or more regulatory sequences (e.g., promoters, enhancers, etc.) and/or intron sequences that, for example, may control or impact one or more aspects of gene expression (e.g., cell-type-specific expression, inducible expression, etc.). As used herein, the term “gene” generally refers to a portion of a nucleic acid that encodes a polypeptide or fragment thereof; the term may optionally encompass regulatory sequences, as will be clear from context to those of ordinary skill in the art. This definition is not intended to exclude application of the term “gene” to non-protein-coding expression units but rather to clarify that, in most cases, the term as used in this document refers to a polypeptide-coding nucleic acid. In some embodiments, a gene may encode a polypeptide, but that polypeptide may not be functional, e.g., a gene variant may encode a polypeptide that does not function in the same way, or at all, relative to the wild-type gene. In some embodiments, a gene may encode a transcript which, in some embodiments, may be toxic beyond a threshold level. In some embodiments, a gene may encode a polypeptide, but that polypeptide may not be functional and/or may be toxic beyond a threshold level.

Genome Editing System: As used herein, the term “genome editing system” refers to any system having DNA editing activity. Among other things, DNA editing activity can include deleting, replacing, or inserting a DNA sequence in a genome. In some embodiments, a genome editing system comprises RNA-guided DNA editing activity. In some embodiments, a genome editing system of the present disclosure includes more than one component. In some embodiments, a genome editing system includes at least two components adapted from naturally occurring CRISPR systems: a guide RNA (gRNA) and an RNA-guided nuclease. In certain embodiments, these two components form a complex that is capable of associating with a specific nucleic acid sequence and editing DNA in or around that nucleic acid sequence, for instance by making one or more of a single-strand break (an SSB or nick), a double-strand break (a DSB) and/or a point mutation. In some embodiments, genome editing systems of the present disclosure lack a component having cleavage activity but maintain a component(s) having DNA binding activity. In some such embodiments, a genome editing system of the present disclosure comprises a component(s) that functions as an inhibitor of DNA activity, e.g., transcription, translation, etc. In some embodiments, a genome editing system of the present disclosure comprises a component(s) fused to modulators to modulate target DNA expression.

Genomic modification: As used herein, the term “genomic modification” refers to a change made in a genomic region of a cell that permanently alters a genome (e.g., an endogenous genome) of that cell. In some embodiments, such changes are in vitro, ex vivo, or in vivo. In some embodiments, every cell in a living organism is modified. In some embodiments, only a particular set of cells such as, e.g., in a specific organ, is modified. For example, in some embodiments, a genome is modified by deletion, substitution, or addition of one or more nucleotides from one or more genomic regions. In some embodiments, a genomic modification is performed in a stem cell or undifferentiated cell. In some such embodiments, progeny of a genomically modified cell or organism will also be genomically modified, relative to a parental genome prior to modification. In some embodiments, a genomic modification is performed on a mature or post-mitotic cell such that no progeny will be generated and thus, no genomic modifications propagated other than in a particular cell.

Heterologous: As used herein, the term “heterologous” may be used in reference to one or more regions of a particular molecule as compared to another region and/or another molecule. For example, in some embodiments, heterologous polypeptide domains, refers to the fact that polypeptide domains do not naturally occur together (e.g., in the same polypeptide). For example, in fusion proteins generated by the hand of man, a polypeptide domain from one polypeptide may be fused to a polypeptide domain from a different polypeptide. In such a fusion protein, two polypeptide domains would be considered “heterologous” with respect to each other, as they do not naturally occur together.

Identity: As used herein, the term “identity” refers to overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of percent identity of two nucleic acid or polypeptide sequences, for example, can be performed by aligning two sequences for optimal comparison purposes (e.g., gaps can be introduced in one or both of a first and a second sequences for optimal alignment and non-identical sequences can be disregarded for comparison purposes). In some embodiments, a length of a sequence aligned for comparison purposes is at least 30%, at least 40%, at least 50%, at least 60%, at least 70%, at least 80%, at least 90%, at least 95%, or substantially 100% of length of a reference sequence; nucleotides at corresponding positions are then compared. When a position in the first sequence is occupied by the same residue (e.g., nucleotide or amino acid) as a corresponding position in the second sequence, then the two molecules (i.e., first and second) are identical at that position. Percent identity between two sequences is a function of the number of identical positions shared by the two sequences being compared, taking into account the number of gaps, and the length of each gap, which needs to be introduced for optimal alignment of the two sequences. Comparison of sequences and determination of percent identity between two sequences can be accomplished using a mathematical algorithm. For example, percent identity between two nucleotide sequences can be determined using the algorithm of Meyers and Miller (CABIOS, 1989, 4:11-17, which is herein incorporated by reference in its entirety), which has been incorporated into the ALIGN program (version 2.0). In some embodiments, nucleic acid sequence comparisons made with the ALIGN program use a PAM120 weight residue table, a gap length penalty of 12 and a gap penalty of 4.

Inhibitory nucleic acid: As used herein, the term “inhibitory nucleic acid” refers to a nucleic acid sequence that hybridizes specifically to a target gene, including target DNA or RNA (e.g., a target mRNA). Thereby, in some embodiments, an inhibitory nucleic acid inhibits expression and/or activity of a target gene. In some embodiments, an inhibitory nucleic acid is a short interfering RNA (siRNA), a short hairpin RNA (shRNA), a microRNA (or “miRNA”), an antisense oligonucleotide, a guide RNA (gRNA), or a ribozyme. In some embodiments, an inhibitory nucleic acid is between about 10 nucleotides to about 30 nucleotides in length (e.g., about 10 nucleotides to about 28 nucleotides, about 10 nucleotides to about 26 nucleotides, about 10 nucleotides to about 24 nucleotides, about 10 nucleotides to about 22 nucleotides, about 10 nucleotides to about 20 nucleotides, about 10 nucleotides to about 18 nucleotides, about 10 nucleotides to about 16 nucleotides, about 10 nucleotides to about 14 nucleotides, about 10 nucleotides to about 12 nucleotides, about 12 nucleotides to about 30 nucleotides, about 12 nucleotides to about 28 nucleotides, about 12 nucleotides to about 26 nucleotides, about 12 nucleotides to about 24 nucleotides, about 12 nucleotides to about 22 nucleotides, about 12 nucleotides to about 20 nucleotides, about 12 nucleotides to about 18 nucleotides, about 12 nucleotides to about 16 nucleotides, about 12 nucleotides to about 14 nucleotides, about 16 nucleotides to about 30 nucleotides, about 16 nucleotides to about 28 nucleotides, about 16 nucleotides to about 26 nucleotides, about 16 nucleotides to about 24 nucleotides, about 16 nucleotides to about 22 nucleotides, about 16 nucleotides to about 20 nucleotides, about 16 nucleotides to about 18 nucleotides, about 18 nucleotides to about 30 nucleotides, about 18 nucleotides to about 28 nucleotides, about 18 nucleotides to about 26 nucleotides, about 18 nucleotides to about 24 nucleotides, about 18 nucleotides to about 22 nucleotides, about 18 nucleotides to about 20 nucleotides, about 20 nucleotides to about 30 nucleotides, about 20 nucleotides to about 28 nucleotides, about 20 nucleotides to about 26 nucleotides, about 20 nucleotides to about 24 nucleotides, about 20 nucleotides to about 22 nucleotides, about 22 nucleotides to about 30 nucleotides, about 22 nucleotides to about 28 nucleotides, about 22 nucleotides to about 26 nucleotides, about 22 nucleotides to about 24 nucleotides, about 24 nucleotides to about 30 nucleotides, about 24 nucleotides to about 28 nucleotides, about 24 nucleotides to about 26 nucleotides, about 26 nucleotides to about 30 nucleotides, about 26 nucleotides to about 28 nucleotides, about 28 nucleotides to about 30 nucleotides, or 11, 12, 13, 14, 15, 16, 17, 18, 19, 20, 21, 22, 23, 24, 25, 26, 27, 28, 29 or 30 nucleotides

Improve, increase, enhance, inhibit or reduce: As used herein, the terms “improve,” “increase,” “enhance,” “inhibit,” “reduce,” or grammatical equivalents thereof, indicate values that are relative to a baseline or other reference measurement. In some embodiments, a value is statistically significantly difference that a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment. In some embodiments, an appropriate reference is a negative reference; in some embodiments, an appropriate reference is a positive reference.

Knockdown: As used herein, the term “knockdown” refers to a decrease in expression of one or more gene products. In some embodiments, an inhibitory nucleic acid achieve knockdown. In some embodiments, a genome editing system described herein achieves knockdown.

Knockout: As used herein, the term “knockout” refers to ablation of expression of one or more gene products. In some embodiments, a genome editing system described herein achieve knockout.

Modulating: As used herein, the term “modulating,” means mediating a detectable increase or decrease in a level of a response in a subject compared with a level of a response in a subject in absence of a treatment or compound, and/or compared with a level of a response in an otherwise identical but untreated subject. The term encompasses perturbing and/or affecting a native signal or response thereby mediating a beneficial therapeutic response in a subject, preferably, a human.

Nuclease: As used herein, the term “nuclease” refers to an agent, for example a protein or a small molecule, capable of cleaving a phosphodiester bond connecting nucleotide residues in a nucleic acid molecule. In some embodiments, a nuclease is a protein, e.g., an enzyme that can bind a nucleic acid molecule and cleave a phosphodiester bond connecting nucleotide residues within a nucleic acid molecule. A nuclease may be an endonuclease, cleaving a phosphodiester bonds within a polynucleotide chain, or an exonuclease, cleaving a phosphodiester bond at the end of the polynucleotide chain. In some embodiments, a nuclease is a site-specific nuclease, binding and/or cleaving a specific phosphodiester bond within a specific nucleotide sequence, which is also referred to herein as the “recognition sequence,” the “nuclease target site,” or the “target site.” In some embodiments, a nuclease is a RNA-guided (i.e., RNA-programmable) nuclease, which complexes with (e.g., binds with) an RNA having a sequence that complements a target site, thereby providing the sequence specificity of a nuclease. In some embodiments, a nuclease recognizes a single stranded target site, while in some embodiments, a nuclease recognizes a double-stranded target site, for example a double-stranded DNA target site. Target sites of many naturally occurring nucleases, for example, many naturally occurring DNA restriction nucleases, are well known to those of skill in the art. In many cases, a DNA nuclease, such as EcoRI, HindIII, or BamHI, recognize a palindromic, double-stranded

DNA target site of 4 to 10 base pairs in length, and cut each of the two DNA strands at a specific position within a target site. Some endonucleases cut a double-stranded nucleic acid target site symmetrically, i.e., cutting both strands at the same position so that the ends comprise base-paired nucleotides, also referred to herein as blunt ends. Other endonucleases cut a double-stranded nucleic acid target sites asymmetrically, i.e., cutting each strand at a different position so that the ends comprise unpaired nucleotides. Unpaired nucleotides at an end of a double-stranded DNA molecule are also referred to as “overhangs,” e.g., as “5′-overhang” or as “3′-overhang,” depending on whether unpaired nucleotide(s) form(s) the 5′ or the 3′ end of a given DNA strand. Double-stranded DNA molecule ends ending with unpaired nucleotide(s) are also referred to as sticky ends, as they can “stick to” other double-stranded DNA molecule ends comprising complementary unpaired nucleotide(s). A nuclease protein typically comprises a “binding domain” that mediates interaction of a protein with a nucleic acid substrate, and also, in some cases, specifically binds to a target site, and a “cleavage domain” that catalyzes the cleavage of a phosphodiester bond within a nucleic acid backbone. In some embodiments, a nuclease protein can bind and cleave a nucleic acid molecule in a monomeric form, while, in some embodiments, a nuclease protein has to dimerize or multimerize in order to cleave a target nucleic acid molecule. Binding domains and cleavage domains of naturally occurring nucleases, as well as modular binding domains and cleavage domains that can be fused to create nucleases binding specific target sites, are well known to those of skill in the art.

Nucleic acid: As used herein, the term “nucleic acid”, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments, a nucleic acid has a nucleotide sequence comprising at least one element that encodes, or is complementary to a sequence that encodes, a polypeptide. In some embodiments, a nucleic acid has enzymatic activity.

Operably linked: As used herein, refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with coding elements of interest; in some embodiments, control elements act in trans to or otherwise at a from the functional element of interest. In some embodiments, “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. In some embodiments, for example, a functional linkage may include transcriptional control. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

Pharmaceutical composition: As used herein, the term “pharmaceutical composition” refers to a composition in which an active agent is formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for, e.g., administration, for example, an injectable formulation that is, e.g., an aqueous or non-aqueous solution or suspension or a liquid drop designed to be administered into an ear canal. In some embodiments, a pharmaceutical composition may be formulated for administration via injection either in a particular organ or compartment, e.g., directly into an ear, or systemic, e.g., intravenously. In some embodiments, a formulation may be or comprise drenches (aqueous or non-aqueous solutions or suspensions), tablets, boluses, powders, granules, pastes, capsules, powders, etc. In some embodiments, an active agent may be or comprise an isolated, purified, or pure compound.

Pharmaceutically acceptable: As used herein, the term “pharmaceutically acceptable” which, for example, may be used in reference to a carrier, diluent, or excipient used to formulate a pharmaceutical composition as disclosed herein, means that a carrier, diluent, or excipient is compatible with other ingredients of a composition and not deleterious to a recipient thereof.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting a subject compound from one organ, or portion of a body, to another organ, or portion of a body. Each carrier must be is “acceptable” in the sense of being compatible with other ingredients of a formulation and not injurious to a patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Polypeptide: As used herein, the term “polypeptide” refers to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may include one or more pendant groups or other modifications, e.g., modifying or attached to one or more amino acid side chains, at a polypeptide's N-terminus, at a polypeptide's C-terminus, or any combination thereof. In some embodiments, such pendant groups or modifications may be acetylation, amidation, lipidation, methylation, pegylation, etc., including combinations thereof. In some embodiments, polypeptides may contain L-amino acids, D-amino acids, or both and may contain any of a variety of amino acid modifications or analogs known in the art. In some embodiments, useful modifications may be or include, e.g., terminal acetylation, amidation, methylation, etc. In some embodiments, a protein may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, a protein is antibodies, antibody fragments, biologically active portions thereof, and/or characteristic portions thereof.

Polynucleotide: As used herein, the term “polynucleotide” refers to any polymeric chain of nucleic acids. In some embodiments, a polynucleotide is or comprises RNA; in some embodiments, a polynucleotide is or comprises DNA. In some embodiments, a polynucleotide is, comprises, or consists of one or more natural nucleic acid residues. In some embodiments, a polynucleotide is, comprises, or consists of one or more nucleic acid analogs. In some embodiments, a polynucleotide analog differs from a nucleic acid in that it does not utilize a phosphodiester backbone. Alternatively or additionally, in some embodiments, a polynucleotide has one or more phosphorothioate and/or 5′-N-phosphoramidite linkages rather than phosphodiester bonds. In some embodiments, a polynucleotide is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a polynucleotide is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a polynucleotide comprises one or more modified sugars (e.g., 2′-fluororibose, ribose, 2′-deoxyribose, arabinose, and hexose) as compared with those in natural nucleic acids. In some embodiments, a polynucleotide has a nucleotide sequence that encodes a functional gene product such as an RNA or protein. In some embodiments, a polynucleotide includes one or more introns. In some embodiments, a polynucleotide is prepared by one or more of isolation from a natural source, enzymatic synthesis by polymerization based on a complementary template (in vivo or in vitro), reproduction in a recombinant cell or system, and chemical synthesis. In some embodiments, a polynucleotide is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a polynucleotide is partly or wholly single stranded; in some embodiments, a polynucleotide is partly or wholly double stranded. In some embodiments, a polynucleotide has a nucleotide sequence comprising at least one element that encodes, or is the complement of a sequence that encodes, a polypeptide. In some embodiments, a polynucleotide has enzymatic activity.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a genotypic variant thereof. Those of ordinary skill will appreciate that a protein can sometimes include more than one polypeptide chain, for example linked by one or more disulfide bonds or associated by other means.

Recombinant: As used herein, the term “recombinant” is intended to refer to polypeptides that are designed, engineered, prepared, expressed, created, manufactured, and/or or isolated by recombinant means, such as polypeptides expressed using a recombinant expression construct transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of a polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control. In some embodiments, a reference is a negative control reference; in some embodiments, a reference is a positive control reference.

Regulatory Element: As used herein, the term “regulatory element” or “regulatory sequence” refers to non-coding regions of DNA that regulate, in some way, expression of one or more particular genes. In some embodiments, such genes are apposed or “in the neighborhood” of a given regulatory element. In some embodiments, such genes are located quite far from a given regulatory element. In some embodiments, a regulatory element impairs or enhances transcription of one or more genes. In some embodiments, a regulatory element may be located in cis to a gene being regulated. In some embodiments, a regulatory element may be located in trans to a gene being regulated. For example, in some embodiments, a regulatory sequence refers to a nucleic acid sequence which is regulates expression of a gene product operably linked to a regulatory sequence. In some such embodiments, this sequence may be an enhancer sequence and other regulatory elements which regulate expression of a gene product.

Sample: As used herein, the term “sample” typically refers to an aliquot of material obtained or derived from a source of interest. In some embodiments, a source of interest is a biological or environmental source. In some embodiments, a source of interest may be or comprise a cell or an organism, such as a microbe (e.g., virus), a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchioalveolar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Subject: As used herein, the term “subject” refers an organism, typically a mammal (e.g., a human, in some embodiments including prenatal human forms). In some embodiments, a subject is a non-human primate. In some embodiments a non-human primate is a cynomolgus macaque. In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to a qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture a potential lack of completeness inherent in many biological and chemical phenomena.

Target site: As used herein, the term “target site” means a portion of a nucleic acid to which a binding molecule, e.g., a microRNA, an siRNA, a guide RNA (“gRNA”) or a guide RNA: Cas complex, will bind, provided sufficient conditions for binding exist. In some embodiments, a nucleic acid comprising a target site is double stranded. In some embodiments, a nucleic acid comprising a target site is single stranded. Typically, a target site comprises a nucleic acid sequence to which a binding molecule, e.g., a gRNA or a gRNA: Cas complex described herein, binds and/or that is cleaved as a result of such binding. In some embodiments, a target site comprises a nucleic acid sequence (also referred to herein as a target sequence or protospacer) that is complementary to a DNA sequence to which the targeting sequence (also referred to herein as the spacer) of a gRNA described herein binds. In some embodiments in the context of RNA-guided nucleases, e.g., CRISPR/Cas nucleases, a target site typically comprises a nucleotide sequence (also referred to herein as a target sequence or a protospacer) that is complementary to a sequence comprised in a gRNA (also referred to herein as the targeting sequence or the spacer) of an RNA-programmable nuclease. In some such embodiments, a target site further comprises a protospacer adjacent motif (PAM) at the 3′ end or 5′ end adjacent to the gRNA-complementary sequence. For an RNA-guided nuclease Cas9, a target sequence may be, in some embodiments, 16-24 base pairs plus a 3-6 base pair PAM (e.g., NNN, wherein N represents any nucleotide). Exemplary PAM sequences for RNA-guided nucleases, such as Cas9, are known to those of skill in the art and include, without limitation, NNG, NGN, NAG, NGA, NGG, NGAG and NGCG wherein N represents any nucleotide. In addition, Cas9 nucleases from different species have been described, e.g., S. thermophilus recognizes a PAM that comprises the sequence NGGNG, and Cas9 from S. aureus recognizes a PAM that comprises the sequence NNGRRT. In some embodiments, Cas9 from S. aureus recognizes a PAM that comprises the sequence NNNRRT. Additional PAM sequences are known in the art, including, but not limited to NNAGAAW and NAAR (see, e.g., Esvelt and Wang, Molecular Systems Biology, 9:641 (2013), the entire content of which is incorporated herein by reference). For example, the target site of an RNA-guided nuclease, such as, e.g., Cas9, may comprise a structure [Nz]-[PAM], where each Nis, independently, any nucleotide, and z is an integer between 1 and 50. In some embodiments, z is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50. In some embodiments, z is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48,49, or 50. In some embodiments, Z is 20.

Treatment: As used herein, the term “treatment” (also “treat” or “treating”) refers to any administration of a therapy that partially or completely alleviates, ameliorates, eliminates, reverses, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of a given disease, disorder, and/or condition.

Variant: As used herein, the term “variant” refers to a version of something, e.g., a gene sequence, that is different, in some way, from another version. To determine if something is a variant, a reference version is typically chosen and a variant is different relative to that reference version. In some embodiments, a variant can have the same or a different (e.g., increased or decreased) level of activity or functionality than a wild type sequence. For example, in some embodiments, a variant can have improved functionality as compared to a wild-type sequence if it is, e.g., mutated to confer reduced toxicity in a cell. As another example, in some embodiments, a variant can have improved functionality as compared to a wild-type sequence if it is, e.g., mutated to confer improved protein production in a cell.

### BRIEF DESCRIPTION OF THE DRAWING

FIG. 1 shows alignments of an N-terminus region of exemplary protoparvovirus VP1u within a VP1 capsid polypeptide. Alignments depicted by FIG. 1 reveal significant conservation of a stretch of amino acid residues (or amino acid motif) within exemplary protoparvovirus species including bufavirus (BuV), cutavirus (CuV), tusavirus (TuV), minute virus of mice (MVM), canine parvovirus (CPV), and feline panleukopenia virus (FPV). Alignments depicted by FIG. 1 also show significant conservation of a putative nuclear localization signal (NLS) upstream of a five amino acid motif. Alignments depicted by FIG. 1 also show highly conserved PLA2 motif residues downstream of an amino acid motif.

FIG. 2 shows alignments of highly conserved parvovirus PLA2 motif residues.

FIG. 3 shows an image depicting adjacent splice donor/acceptor sequences between a NLS (KRARRG-SEQ ID NO: 145) and initiation of a PLA2 motif that results in deletion of a five amino acid motif in a reference canine parvovirus (CPV) VP1 capsid polypeptide sequence, according to an embodiment of the present disclosure.

FIG. 4 shows an image depicting two adjacent donor/acceptor sequences between a NLS (KRAKRG-SEQ ID NO: 146) and a PLA2 motif that can result in deletion of a five amino acid motif in a reference minute virus of mice (MVM) VP1 capsid polypeptide sequence, according to an embodiment of the present disclosure.

FIG. 5 shows an image depicting adjacent splice acceptor/donor sequences between a NLS (KRAKRG-SEQ ID NO: 146) and a PLA2 motif that can result in deletion of a five amino acid motif in a reference rat H-1 parvovirus (H-1PV) VP1 capsid polypeptide sequence, according to an embodiment of the present disclosure.

FIG. 6 shows an image depicting adjacent donor/acceptor sequences between a NLS (KARG-SEQ ID NO: 147) and a PLA2 motif that can result in deletion or partial deletion of a five amino acid motif in a reference cutavirus (CuV) VP1 capsid polypeptide sequence, according to an embodiment of the present disclosure.

FIG. 7 shows a schematic depicting deletion of a five amino acid motif in a VP1u region of a canine parvovirus (CPV) VP1 capsid polypeptide, according to an embodiment of the present disclosure. In some embodiments, such a deletion leads to reduced toxicity in insect cells, high capsid yield. Moreover, the present disclosure describes that this approach can be applied to other protoparvoviruses.

FIG. 8 shows a graph demonstrating that a canine parvovirus (CPV) reference VP1 capsid polypeptide (1) exhibited elevated toxicity in insect cells at 72 hours post-infection (hpi), and (2) affected VP1 capsid polypeptide yield, compared to other genuses in family parvovirinae (such as bocavirus or erythroparvovirus), according to an embodiment of the present disclosure.

FIG. 9 shows a graph demonstrating that a canine parvovirus (CPV) variant VP1 capsid polypeptide exhibited more than double the average percent cell viability at 72 hpi compared to a CPV reference VP1 capsid polypeptide, according to an embodiment of the present disclosure.

FIG. 10 shows a Western Blot that measured levels of canine parvovirus (CPV) VP1 capsid polypeptide and VP2 capsid polypeptide in the supernatant and pellet of insect (Sf9) cells infected with a baculovirus construct (BEV) comprising a CPV variant VP1 capsid coding sequence, according to an embodiment of the present disclosure.

FIG. 11 depicts exemplary protoparvovirus construct elements that can improve production and/or reduce toxicity of a protoparvovirus variant VP1 capsid polypeptide in host cells, according to an embodiment of the present disclosure.

FIG. 12 shows a schematic that depicts alternative initiation of a VP1 capsid polypeptide leads to a longer or shorter VP1 capsid polypeptide which can negatively impact virion potency, according to an embodiment of the present disclosure.

FIG. 13 shows a schematic depicting models for involvement of AAV Rep helicases as motors to incorporate a viral genome into a preformed capsid, as (A) a single-stranded molecule using the initial ‘scanning’ function before the first duplexed base pairs are encountered or (B) by unwinding a double-stranded dimer or multimer genome on a capsid surface at the same time or (C) simultaneous replication (arrow) of a double-stranded monomer genome being packaged, according to an embodiment of the present disclosure.

FIG. 14 shows virion yields (vg/mL) of virions comprising a CPV VP1 capsid polypeptide encoded by a VP1 capsid coding sequence according to SEQ ID NO: 148 (Exemplary CPV Construct 7) produced in host HEK293 cells, virions comprising a CPV VP1 capsid polypeptide encoded by a VP1 capsid coding sequence according to SEQ ID NO: 130 (Exemplary CPV Construct 5) produced in host HEK293 cells, virions comprising a CuV VP1 capsid polypeptide encoded by a VP1 capsid coding sequence according to SEQ ID NO: 139 (Exemplary CuV Construct 6) produced in host HEK293 cells, virions comprising a CuV VP1 capsid polypeptide encoded by a VP1 capsid coding sequence according to SEQ ID NO: 133 (Exemplary CuV Construct 3) produced in host HEK293 cells, virions comprising a CuV VP1 capsid polypeptide encoded by a VP1 capsid coding sequence according to SEQ ID NO: 134 (Exemplary CuV Construct 4) produced in host HEK293 cells, and virions comprising an exemplary control HBoV1 capsid polypeptide produced in host HEK293 cells.

FIG. 15A shows virion density of virions (or particles) that were detected and isolated via ultracentrifugation in CsCl of virions comprising a CPV VP1 capsid polypeptide encoded by a VP1 capsid coding sequence according to SEQ ID NO: 148 (Exemplary CPV Construct 7), and virions comprising a CPV VP1 capsid polypeptide encoded by a VP1 capsid coding sequence according to SEQ ID NO: 130 (Exemplary CPV Construct 5).

FIG. 15B shows a western blot analysis of capsid composition and amounts of VP1 and VP2 capsid polypeptides of virions comprising a CPV VP1 capsid polypeptide encoded by a VP1 capsid coding sequence according to SEQ ID NO: 148 (Exemplary CPV Construct 7), and virions comprising a CPV VP1 capsid polypeptide encoded by a VP1 capsid coding sequence according to SEQ ID NO: 130 (Exemplary CPV Construct 5) produced in host HEK293 cells.

FIG. 16A shows virion density of virions (or particles) that were detected and isolated via ultracentrifugation in CsCl of virions comprising a CuV VP1 capsid polypeptide encoded by a VP1 capsid coding sequence according to SEQ ID NO: 139 (Exemplary CuV Construct 6) produced in HEK293 cells.

FIG. 16B shows a western blot analysis of capsid composition and amounts of VP1 and VP2 capsid polypeptides of virions comprising a CuV VP1 capsid polypeptide encoded by a VP1 capsid coding sequence according to SEQ ID NO: 139 (Exemplary CuV Construct 6) produced in host HEK293 cells.

FIG. 17A shows virion density of virions (or particles) that were detected and isolated via ultracentrifugation in CsCl of virions, a CuV VP1 capsid polypeptide encoded by a VP1 capsid coding sequence according to SEQ ID NO: 133 (Exemplary CuV Construct 3) produced in HEK293 cells.

FIG. 17B shows a western blot analysis of capsid composition and amounts of VP1 and VP2 capsid polypeptides of virions comprising a CuV VP1 capsid polypeptide encoded by a VP1 capsid coding sequence according to SEQ ID NO: 133 (Exemplary CuV Construct 3) produced in host HEK293 cells.

FIG. 18A shows virion density of virions (or particles) that were detected and isolated via ultracentrifugation in CsCl of virions comprising a CuV VP1 capsid polypeptide encoded by a VP1 capsid coding sequence according to SEQ ID NO: 134 (Exemplary CuV Construct 4) produced in HEK293 cells.

FIG. 18B shows a western blot analysis of capsid composition and amounts of VP1 and VP2 capsid polypeptides of virions comprising a CuV VP1 capsid polypeptide encoded by a VP1 capsid coding sequence according to SEQ ID NO: 134 (Exemplary CuV Construct 4) produced in host HEK293 cells.

FIG. 19 shows a schematic depicting a structural model of interaction between a virion comprising a protoparvovirus VP1 capsid polypeptide encoded by a VP1 capsid coding sequence described herein and a transferrin receptor (TfR).

FIG. 20 shows fluorescence imaging of human neuroblastoma cell line SH-SY5Y cells (left) and kidney cell line HEK293 cells (right) transduced with MOI 1E+4 vg/cell of virions comprising a CPV VP1 capsid polypeptide encoded by a VP1 capsid coding sequence according to SEQ ID NO: 126 (Exemplary CPV Construct 1).

FIG. 21 shows a bar graph depicting virion yields (vg/mL) of virions comprising a CPV VP1 capsid polypeptide encoded by a VP1 capsid coding sequence according to SEQ ID NO: 130 (Exemplary CPV Construct 5) produced in host HEK293T cells, across three independent experiments.

FIG. 22 shows (left) a western blot analysis of capsid composition and amounts of VP1 and VP2 capsid polypeptides of virions comprising a CPV VP1 capsid polypeptide encoded by a VP1 capsid coding sequence according to SEQ ID NO: 130 (Exemplary CPV Construct 5) with (+) and without (−) trypsin treatment conditions, produced in host HEK293 cells and (right) a western blot analysis of capsid composition and amounts of VP1, a VP2 cleavage product (VP2′), and VP2 capsid polypeptides of virions comprising a CPV VP1 capsid polypeptide encoded by a VP1 capsid coding sequence according to SEQ ID NO: 130 (Exemplary CPV Construct 5).

FIG. 23 shows fluorescence imaging of kidney cell line HEK293T cells transduced with MOI 1E4, 1E3, 1E2 vg/cell of virions comprising a CPV VP1 capsid polypeptide encoded by a VP1 capsid coding sequence according to SEQ ID NO: 130 (Exemplary CPV Construct 5) with (+) and without (−) trypsin treatment conditions. Imaging was performed at 2 days and 6 hours.

FIG. 24 shows a bar graph depicting GFP transgene expression as measured by GCU×μm2 per image of HEK293T cells transduced with MOI 1E4 vg/cell, 1E3 vg/cell, and 1E2 vg/cell of virions comprising a CPV VP1 capsid polypeptide encoded by a VP1 capsid coding sequence according to SEQ ID NO: 130 (Exemplary CPV Construct 5) with (+) and without (−) trypsin treatment conditions. Measurements were quantified via Incucyte.

### DETAILED DESCRIPTION OF CERTAIN EMBODIMENTS

Among other things, the present disclosure recognizes that compositions, preparations, constructs, virions, population of virions, and host cells comprising a protoparvovirus variant VP1 capsid polypeptide are particularly advantageous as a vehicle for gene therapy.

First, due to a larger virion genome size, a protoparvovirus (˜5.3 kb (e.g., canine parvovirus) compared with ˜4.7 kb of AAV) can package a nucleic acid at least 0.6 kb greater than AAV, thereby allowing delivery of a therapeutic gene(s) whose size exceeds the capacity of AAV. A larger virion genome size also allows delivery of a therapeutic transgene(s) together with genomic safe harbor (GSH) sequences that accommodate site-specific recombination of the transgene(s) at a desired genomic location. Such site-specific recombination allows integration of the transgene at an inert location in the genome, as opposed to random integration that could disrupt an essential gene and its expression.

Second, unlike AAV, protoparvovirus is not as prevalent as AAV. Thus, administration of a virion comprising a protoparvovirus variant VP1 capsid polypeptide would not trigger an extensive anti-viral immune reaction that precludes efficient gene delivery. That is, in some embodiments, no prescreening of a subject for anti-parvovirus antibodies is required prior to administering (e.g., systemically) compositions (e.g., pharmaceutical compositions), preparations, constructs, virions, population of virions described herein. Accordingly, a virion comprising a protoparvovirus variant VP1 capsid polypeptide can achieve gene delivery with the efficiency unparalleled to AAV.

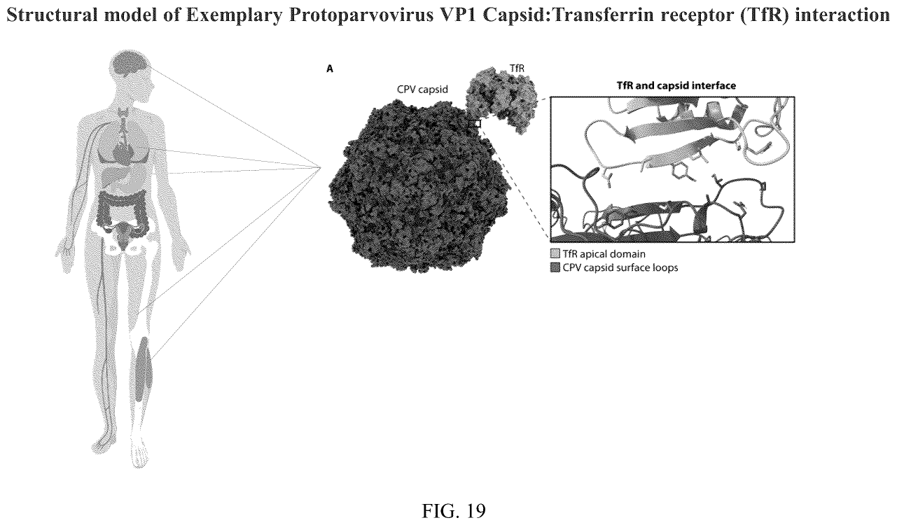

Third, protoparvovirus has an extraordinary tropism for specific tissues. For example, protoparvovirus has a tropism for hematopoietic stem cells and is particularly useful for treatment or prevention of hematologic diseases such as hemoglobinopathies, anemia, myeloproliferative disorders, coagulopathies, and cancer. In addition, protoparvovirus can efficiently transcytose across the cells via its interaction with a transferrin receptor. Thus, protoparvovirus can cross a blood-brain barrier (BBB) and deliver therapeutic genes to nerve cells that are hidden behind an endothelial barrier (see, e.g., FIG. 19, see also, e.g., Lopez-Atacio, et al., J. Virol. (2023), the contents of which are incorporated by reference herein in its entirety).

It is an insight of the present disclosure that a model of capsid: TfR interaction and capsid: TfR binding (e.g., as described by Lopez-Atacio, et al., J. Virol. (2023) can be extended to a protoparvovirus described herein. It is an insight of the present disclosure that a model of capsid: TfR interaction and capsid: TfR binding (e.g., as described by Lopez-Atacio, et al., J. Virol. (2023)) can be extended to a canine protoparvovirus described herein. Further, it is an insight of the present disclosure that a VP1 capsid polypeptide encoded by a VP1 capsid coding sequence described herein exhibits a capsid: TfR interaction and capsid: TfR interaction binding (see, e.g., FIG. 19). Among other things, in some embodiments, interaction with a TfR receptor results in cell-specific tropism. Also, as described herein, TfR is a receptor of interest for blood-brain barrier (BBB)-transcytosis-mediated CNS delivery. For example, in some embodiments, virions comprising a VP1 capsid polypeptide encoded by a VP1 capsid coding sequence described herein exhibit cell-specific tropism for central nervous system (CNS) cells. As another example, in some embodiments, virions comprising a VP1 capsid polypeptide encoded by a VP1 capsid coding sequence described herein exhibit cell-specific tropism for kidney cells. As another example, in some embodiments, virions comprising a VP1 capsid polypeptide encoded by a VP1 capsid coding sequence described herein exhibit cell-specific tropism for lung cells. As another example, in some embodiments, virions comprising a VP1 capsid polypeptide encoded by a VP1 capsid coding sequence described herein exhibit cell-specific tropism for bone marrow cells. As another example, in some embodiments, virions comprising a VP1 capsid polypeptide encoded by a VP1 capsid coding sequence described herein exhibit cell-specific tropism for muscle cells. Accordingly, a virion comprising a capsid protein of protoparvovirus provides a novel means of gene therapy for patients afflicted with e.g., neurodegenerative or neuromuscular diseases. Accordingly, a virion comprising protoparvovirus capsid protein(s) provides a new modality for gene therapy that can target specific cells/tissues/organs for treatment or prevention of a wide range of human diseases.

Protoparvovirus capsid polypeptides comprise two main structural polypeptides, VP1, with an approximate MW of 81 KDa, and VP2 with an approximate MW of 58 to 62 KDa. In some embodiments, viral capsid polypeptide stoichiometry is VP1: VP2 (from about 1:10 to about 1:20, e.g., about 1:10, 1:11, 1:12, 1:13, 1:14, 1:15, 1:16, 1:17, 1:18, 1:19, 1:20).

For example, in some embodiments, the present disclosure recognizes that a protoparvovirus VP1 capsid polypeptide (e.g., within a VP1 unique region (VP1u)) harbors amino acid residues that are useful for virion internalization. Moreover, among other things, the present disclosure recognizes that a protoparvovirus VP1 harbors amino acid motifs that are useful for transit to a cell nucleus. Additionally, among other things, the present disclosure recognizes that a protoparvovirus VP1 harbors amino acid motifs that are useful for productive virus infection. Moreover, among other things, the present disclosure recognizes that a protoparvovirus phospholipase A (PLA) motif allows for endosomal escape early during infection. For different protoparvovirus species, for example, a N-termini of a protoparvovirus VP1 also harbors stretches of basic amino acids that function as nuclear localization sites (also referred to as nuclear localization signals) (NLS) which can be recognized by importin proteins (alpha, and beta) in host cells. In some embodiments, recognition by importin proteins mediate nuclear delivery (Mantyla et al. 2020, Lyi et al. 2014, each of which is hereby incorporated by reference herein in its entirety).

For example, as described herein, in some embodiments, expression of protoparvovirus full capsid polypeptides (composed of VP1 and VP2) in baculovirus-Sf9 systems has been reported to be challenging, for example, due to cell toxicity. Without wishing to be bound to any theory, it is believed that cell toxicity is presumably a result of protoparvovirus VP1 capsid polypeptide retention in cell cytoplasm, ultimately resulting in protein aggregation and subsequent toxicity (Yuan et al. 2001, the contents of which is hereby incorporated by reference herein in its entirety). Moreover, in some embodiments, differential phosphorylation of MVM capsid (VP1) by host Raf1 kinase led to VP1 capsid polypeptide retention in the cytoplasm (Riobolos et al. 2009, the contents of which is hereby incorporated by reference herein in its entirety). Without wishing to be bound to any theory, it is believed that phosphorylation does not occur in insect cells due to a different sequence and structure from mammalian Raf1.

Moreover, in some embodiments, the present disclosure recognizes splicing events found in a protoparvovirus VP1 capsid polypeptide (e.g., within a VP1u) that eliminates five amino acid residues downstream of an NLS. It is an insight of the present disclosure that these five amino acid residues are conserved across protoparvovirus species. Surprisingly, in some embodiments, the present disclosure describes that this deletion resulted in significant improvement of protoparvovirus VP1 capsid polypeptide expression in a host cell. In some embodiments, a host cell is an insect cell. In some embodiments, an insect cell is a Sf9 cell. In some embodiments, a host cell is a mammalian cell.

### 1. Protoparvovirus

Among other things, the present disclosure describes compositions, preparations, constructs, virions, population of virions, and host cells comprising a protoparvovirus variant VP1 capsid polypeptide relative to a protoparvovirus reference VP1 capsid polypeptide. As described herein, protoparvovirus is of particular interest as a gene therapy composition. For example, neutralizing antibodies against human protoparvovirus, including bufavirus, tusavirus, and cutavirus have low prevalence in many Western countries (Vaisanen, Mohanraj et al. 2018, the entire contents of which are hereby incorporated by reference herein). While circulation of human protoparvovirus, inferred by the prevalence of virus-specific antibodies, has shown to be greater than 50% in the Middle East or Africa, circulation in European countries and in the United States is strikingly low, varying between 0% and 5% (Vaisanen, Mohanraj et al. 2018, the entire contents of which are hereby incorporated by reference herein). This is a feature that makes protoparvovirus particularly attractive for gene therapy as compared to AAV-derived vectors, which has a human IgG prevalence of 40-70%.

Moreover, protoparvovirus has capacity to encapsulate and deliver a larger nucleic acid molecule as compared to AAV-derived vectors. For example, bufavirus can incorporate DNA molecules of ˜5.1 Kb, allowing design and delivery of genomes that encode larger proteins or contain cis-acting regulatory elements in these vectors (when compared to AAV), while tusavirus and cutavirus can incorporate a genome similar to AAV (˜4.6 Kb).

Further, protoparvovirus can target certain cell types, tissues, and/or organs. Human bufavirus and tusavirus have been isolated from respiratory and gastrointestinal (GI) tracks (or stool) in humans, and studies performed in non-human primates suggest that bufavirus can elicit a systemic infection (Vaisanen, Mohanraj et al. 2018, the entire contents of which are hereby incorporated by reference herein). Accordingly, in some embodiments, bufavirus can be used for gene therapy targeting different human organs including but not limited to small intestine, liver, heart, lung, brain, and muscle. In addition, parvovirus capsid polypeptides can tolerate harsh environmental conditions such as low pH levels or physiological conditions found in stomach. Such tolerance makes a virion comprising a protoparvovirus capsid polypeptide(s) suitable for transducing cells of gastrointestinal track, including intestinal stem cells. The small intestine epithelium is organized into two fundamental structures: villi and crypts. Villi form functional absorptive units populated by a diverse group of differentiated cells, including enterocytes, goblet, enteroendocrine, tuft, and microfold cells. Each villus is supported by at least six invaginations, or crypts of Lieberkuhn (Clevers 2013, the entire contents of which are hereby incorporated by reference herein). Crypts are occupied mainly by undifferentiated cells, including transit-amplifying cells; however, differentiated enteroendocrine and Paneth cells also reside in crypts. Wedged between Paneth cells are crypt base columnar cells, which maintain homeostasis through both self-renewal and continuous replacement of differentiated cells that are constantly turned-over. Targeting intestinal stem cells with a virion comprising a protoparvovirus variant capsid(s) of the present disclosure, therefore, opens a possibility to prevent or treat different GI related complications including hereditary hemochromatosis, or inflammatory bowel disease. Use of validated genomic safe harbors for targeting a transgene in intestinal stem cells is substantially beneficial for providing a long-term expression and avoiding any differentiation effect that is often associated with random genomic insertion.

In some embodiments, a protoparvovirus is of a species selected from Carnivore protoparvovirus, Carnivore protoparvovirus 1, Chiropteran protoparvovirus 1, Eulipotyphla protoparvovirus 1, Primate protoparvovirus 1, Primate protoparvovirus 2, Primate protoparvovirus 3, Primate protoparvovirus 4, Rodent protoparvovirus 1, Rodent protoparvovirus 2, Rodent protoparvovirus 3, Ungulate protoparvovirus 1, and Ungulate protoparvovirus 2. In some embodiments, the protoparvovirus is selected from canine parvovirus, feline panleukopenia virus, human bufavirus 1, human bufavirus 2, human bufavirus 3, human tusavirus, human cutavirus, Wuharv parvovirus, porcine parvovirus, minute virus of mice, megabat bufavirus, and a genotypic variant thereof.

a. Characteristic Sequence Elements

Among other things, in some embodiments, the present disclosure recognizes that one or more characteristic sequence elements of a protoparvovirus variant VP1 capsid polypeptide surprisingly affects virion internalization into a host cell, relative to a protoparvovirus reference VP1 capsid polypeptide. Among other things, in some embodiments, the present disclosure recognizes that one or more characteristic sequence elements of a protoparvovirus variant VP1 capsid polypeptide surprisingly affects virion transit into a nucleus of a cell, relative to a protoparvovirus reference VP1 capsid polypeptide. Among other things, the present disclosure recognizes that one or more characteristic sequence elements of a protoparvovirus variant VP1 capsid polypeptide surprisingly affects productive virus infection, relative to a protoparvovirus reference VP1 capsid polypeptide.

i. VP1 Sequence Elements

Among other things, the present disclosure recognizes that a protoparvovirus reference VP1 capsid polypeptide comprises at least three characteristic sequence elements within a protoparvovirus VP1 capsid polypeptide (e.g., within a VP1 unique region (VP1u)). In some embodiments, a protoparvovirus reference VP1 capsid polypeptide comprises a VP1 Sequence Element 1, a VP1 Sequence Element 2, a VP1 Sequence Element 3, or any combination thereof. In some embodiments, a characteristic sequence element is a VP1 Sequence Element 1 as described herein. In some embodiments, a characteristic sequence element is a VP1 Sequence Element 2 as described herein. In some embodiments, a characteristic sequence element is a VP1 Sequence Element 3 as described herein.

In some embodiments, a VP1 Sequence Element 1 functions as a nuclear localization signal sequence (NLS). In some embodiments, a VP1 Sequence Element 2 comprises a stretch of one or more amino acids downstream of a NLS. In some embodiments, a VP1 Sequence Element 3 comprises a PLA2 motif. In some embodiments, a VP1 Sequence Element 2 comprises a stretch of one or more amino acids upstream of a VP1 Sequence Element 3. In some embodiments, a VP1 Sequence Element 2 is between a VP1 Sequence Element 1 and a VP1 Sequence Element 3.

In some embodiments, VP1 Sequence Element 1 comprises a stretch of amino acids that function as a nuclear localization signal sequence (NLS). In some embodiments, Sequence Element 1 comprises a basic structure: (K/I)RARRG. In some embodiments, Sequence Element 1 comprises a basic structure: KARG. In some embodiments, Sequence Element 1 comprises one or more of a K residue, an A residue, an R residue, a G residue, or a combination thereof.

In some embodiments, VP1 Sequence Element 2 comprises a stretch of five amino acids downstream of Sequence Element 1. In some embodiments, VP1 Sequence Element 2 comprises a stretch of five amino acids immediately downstream of Sequence Element 1. In some embodiments, VP1 Sequence Element 2 comprises a stretch of more than five amino acids downstream of Sequence Element 1. In some embodiments, VP1 Sequence Element 2 comprises a stretch of more than five amino acids immediately downstream of Sequence Element 1. In some embodiments, Sequence Element 2 comprises a basic structure: LVPPG (SEQ ID NO: 1). In some embodiments, Sequence Element 2 comprises one or more of an L residue, a V residue, a P residue, a G residue, or a combination thereof. In some embodiments, Sequence Element 2 comprises a basic structure: WVPPG (SEQ ID NO: 2). In some embodiments, Sequence Element 2 comprises a basic structure: WVPPGYNFLG (SEQ ID NO: 3). In some embodiments, Sequence Element 2 comprises one or more of a W residue, a V residue, a P residue, a G residue, or a combination thereof.

In some embodiments, VP1 Sequence Element 3 comprises a PLA2 motif. In some embodiments, a PLA2 motif comprises a Ca2+ binding loop. In some embodiments, VP1 Sequence Element 3 is downstream VP1 Sequence Element 2. In some embodiments, VP1 Sequence Element 3 is immediately downstream VP1 Sequence Element 2. In some embodiment, Sequence Element 3 has a basic structure: LGPF. In some embodiments, Sequence Element 2 comprises one or more of an L residue, a G residue, a P residue, or a combination thereof.

ii. NS1 Sequence Elements

Among other things, the present disclosure recognizes that members of the genus protoparvovirus encode NS1 proteins that are generally greater than 30% identical to each other at the amino acid sequence level as determined by pairwise sequence alignments (Cotmore S. F., et al. Nov. 9, 2013). Among other things, a member of a genus protoparvovirus encodes an NS1 protein that has greater than 30% identity to an exemplary NS1 amino acid sequence according to SEQ ID NO: 4.

Exemplary Canine Parvovirus (CPV) NS1 Amino Acid
Sequence
(SEQ ID NO: 4)
MSGNQYTEEVMEGVNWLKKHAENEAFSFVFKCDNVQLNGKDVRWNNYTK
PIQNEELTSLIRGAQTAMDQTEEEEMDWESEVDSLAKKQVQTFDALIKK
CLFEVFVSKNIEPNECVWFIQHEWGKDQGWHCHVLLHSKNLQQATGKWL
RRQMNMYWSRWLVTLCSVNLTPTEKIKLREIAEDSEWVTILTYRHKQTK
KDYVKMVHFGNMIAYYFLTKKKIVHMTKESGYFLSTDSGWKFNFMKYQD
RQIVSTLYTEQMKPETVETTVTTAQETKRGRIQTKKEVSIKCTLRDLVS
KRVTSPEDWMMLQPDSYIEMMAQPGGENLLKNTLEICTLTLARTKTAFE
LILEKADNTKLTNFDLANSRTCQIFRMHGWNWIKVCHAIACVLNRQGGK
RNTVLFHGPASTGKSIIAQAIAQAVGNVGCYNAANVNFPFNDCTNKNLI
WIEEAGNFGQQVNQFKAICSGQTIRIDQKGKGSKQIEPTPVIMTTNENI
TIVRIGCEERPEHTQPIRDRMLNIKLVCKLPGDFGLVDKEEWPLICAWL
VKHGFVSTMANYTHHWGKVPEWDENWAEPKIQEGINSPGCKDLKTQAAS
NPQSQDQVLTPLTPDVVDLALEPWSTPDTPIAETANQQSNQLGVTHKDV
QASPTWSEIEADLRAIFTSEQLEEDFRDDLD

Among other things, the present disclosure recognizes that members of a species within genus protoparvovirus can be characterized by encoding an NS1 protein that shares at least 85% identity with a NS1 protein encoded by other members of the species (Cotmore S. F., et al. Nov. 9, 2013, the entire contents of which are hereby incorporated by reference herein). Among other things, the present disclosure recognizes that members of genus protoparvovirus are monophyletic.

The present disclosure also recognizes that genomes of founder protoparvoviruses are distinctive because they contain many reiterations of a tetranucleotide sequence 5′-TGGT-3′ (or its complement 5′-ACCA-3′), which is a modular binding motif of the NS1 duplex DNA recognition site, generally depicted as (TGGT)₂₋₃ (Cotmore et al., 1995, the entire contents of which are hereby incorporated by reference herein). Minute virus of mice NS1 recognizes variably spaced, tandem and inverted, clusters of TGGT motif, allowing it to bind to a wide variety of sequences distributed throughout replicative-form viral DNA. TGGT/ACCA tetranucleotide clusters are also dispersed throughout genomes of new viruses, suggesting significant biological similarities with founder members. For example, in a 4822 nt sequence of bufavirus 1a (human) (JX027296) there are 95 copies of ACCA or TGGT, while in a 4452 nt sequence of a melanoma-associated human cutavirus (KX685945) there are 105 separate copies.

b. Virions

Among other things, the present disclosure describes a virion comprising a protoparvovirus variant VP1 capsid polypeptide comprising at least one sequence variation relative to a protoparvovirus reference VP1 capsid polypeptide. In some embodiments, a virion comprises a protoparvovirus variant VP1 capsid polypeptide and a heterologous nucleic acid sequence.

X-ray reconstructions indicate that first ordered VP residues in protoparvovirus capsid polypeptides are located inside a particle at a base of the 5-fold pore, leaving unresolved VP1 and VP2 N-termini of ˜180 and 37 residues, respectively (Halder et al., 2013, Agbandje-McKenna et al., 1998, Xie and Chapman 1996, the contents of which are hereby incorporated by reference herein in its entirety). A C-terminal region of this unresolved sequence forms a slender glycine-rich chain, present in both VP1 and VP2, which in minute virus of mice (MVM) variant VLPs can be modeled into claw-like densities positioned inside the capsid below the 5-fold channels in some cryoEM reconstructions (Subramanian et al., 2017, the entire contents of which are hereby incorporated by reference herein). However, in X-ray structures of MVM virions, but not empty particles, a first 10 amino acids from a single copy of this sequence (VP2 G37-G28) can be modeled into submolar density that occupies a central pore of most 5-fold cylinders. Although all VP1 and VP2 N-terminal peptides are sequestered in empty particles, a subset of MVM VP2 N-termini become exposed at a virion surface early during genome encapsidation (Cotmore and Tattersall 2005, the entire contents of which are hereby incorporated by reference herein), presumably via a poorly understood conformational shift that involves expansion of the 5-fold cylinders. These externalized VP2 N-termini contain a nuclear export signal (Maroto et al., 2004, the entire contents of which are hereby incorporated by reference herein) that in some cells effectively converts a trafficking-neutral capsid into a nuclear export-competent particle. Virions are released from infected cells in this form (Cotmore and Tattersall 2005, the entire contents of which are hereby incorporated by reference herein), but both in an extracellular environment and during cell entry, exposed N-termini undergo proteolytic cleavage, which removes ˜25 amino acids and converts VP2 to a form called VP3. Because X-ray structures show slightly less than one polyglycine tract threaded through each cylinder, it is significant that ˜90% of the ˜50 MVM VP2 termini eventually become surface exposed and cleaved. X-ray structures of cleaved, predominantly VP3, virions indicate that this proteolysis allows the polyglycine tract of cleaved proteins to be retracted into the capsid interior, where it folds back and assumes additional icosahedral ordering extending to residue G30, while being replaced in cylinders by a new cluster of VP2 N-termini (Govindasamy L, Gurda B L, Halder S, Van Vliet K, McKenna R, Cotmore S F, Tattersall P, Agbandje-McKenna M. 2010, unpublished observations). Externalized VP2 N-termini also serve an important structural role, stabilizing the cylinders prior to cell entry and preventing premature exposure of VP1 N-termini and ultimately the genome (Cotmore and Tattersall 2012). Thus, in members of genus Protoparvovirus, 5-fold cylinders serve as portals for three different forms of cargo, mediating 1) genome translocation into and out of an intact particle, 2) VP1SR extrusion prior to bilayer transit, and 3) early externalization of some VP2 N-termini concomitant with genome encapsidation. This is in sharp contrast to viruses in many other parvovirus genera, which rely on just one or two of these portal functions.

A second distinctive feature of protoparvovirus virions is that in X-ray structures not only is a capsid icosahedrally ordered, but so is ˜11-34% of the single-stranded DNA genome, forming patches in each asymmetric unit that are positioned below a cavity on an interior capsid surface. This ordered DNA comprises 2-3 short (8-11 nt) single-strands, which adopt an inverted-loop configuration with phosphates chelated in interior by two Mg++ ions while bases point outwards towards a capsid shell where they establish non-covalent interactions with specific amino acid side chains (Halder et al., 2013, Agbandje-McKenna et al., 1998, Chapman and Rossmann 1995, the contents of which are hereby incorporated by reference herein in its entirety). For example, atomic force microscopy has been used to probe rigidity of individual MVM particles along their 5-fold, 3-fold and 2-fold symmetry axes, which showed that in empty particles, but not in DNA-containing virions, two-fold axes can be easily distorted by nanoindentation, suggesting that a genome has a major influence on capsid rigidity of this region (Carrasco et al., 2006, the entire contents of which are hereby incorporated by reference herein). Single alanine mutations that did not compromise intracapsid interactions but did disrupt major interactions between a capsid and bound DNA patches, had no effect on empty particles but abrogated a genome-enhanced 2-fold rigidity seen in full particles, indicating that it derives predominantly from these ordered DNA: capsid interactions (Carrasco et al., 2008, the entire contents of which are hereby incorporated by reference herein). This perhaps indicates an importance of a full-length, 5 kb genome in establishing wild-type capsid dynamics, as also suggested by in vitro uncoating studies (Cotmore et al., 2010, the entire contents of which are hereby incorporated by reference herein).

c. Genome Organization and Replication

Protoparvoviruses have heterotelomeric genomes of around 5 kb, flanked by hairpin telomeres of ˜120 nt at their left-end, generally in a single sequence orientation, while a right-end hairpin is ˜250 nt and can be present as either of two inverted-complementary sequences dubbed “flip” and “flop.” Right-end of protoparvovirus genomes can be excised from replication intermediates in a hairpin configuration by hairpin transfer, which in MVM involves binding of NS1 complexes to two separate clusters of (TGGT)₂₋₃ binding sites, one that positions NS1 over a cleavage site (5′-CTATCA-3′) and a second that is ˜120 bp away, at a hairpin axis. For cleavage to occur, NS1 complexes at these two sites must be coordinated, and a origin refolded, by recruiting DNA bending proteins from a host HMGB family, which bind to NS1 and create an essential ˜30 bp double-helical loop in the intervening G-rich origin DNA (Cotmore et al., 2000, the entire contents of which are hereby incorporated by reference herein).

In contrast, origin sequences generated from a left end of this virus are not cleaved in a hairpin configuration because there is a critical TC/GAA mismatch in a hairpin stem. To create an active origin, a left hairpin must be unfolded and copied to form a base-paired junction region that spans adjacent genomes in dimer RF, in which two arms of a hairpin are effectively segregated on either side of a symmetry axis. However, only a TC arm gives rise to an active origin because a dinucleotide serves as a spacer element that is positioned between a NS1 binding site and a binding site for an essential co-factor, called parvovirus initiation factor (PIF, also known as glucocorticoid modulatory element binding protein GMEB). PIF is a heterodimeric host complex that binds to two spaced 5′-ACGT-3′ half sites positioned near an axis of a DNA palindrome. In an active origin, PIF is able to interact with NS1 across a TC dinucleotide, stabilizing its binding to a relatively weak NS1 binding site, but it cannot stabilize NS1 binding to an identical binding site across a GAA trinucleotide in an inactive (GAA) arm (Christensen et al., 2001, the entire contents of which are hereby incorporated by reference herein). In consequence, sequences in the hairpin configuration or perfectly-duplex hairpin arms carrying a GAA sequence are not cleaved, making them potentially available for alternative roles such as driving transcription from an adjacent P4 promoter (Gu et al., 1995, the entire contents of which are hereby incorporated by reference herein). Due to major disparities in cleavage efficiency between a left- and right-end origins, progeny negative-sense single-strands are preferentially displaced from a right end of a genome, with the result that protoparvoviruses typically displace and package predominantly (˜99%) negative-sense progeny ssDNA.

Viruses in this genus use two transcriptional promoters at map units (mu) 4 and 38, and a single polyadenylation site corresponding to mu 95, to create 3 major size classes of mRNAs, all of which have a short intron sequence between 46-48 mu removed (Pintel et al., 1983, the entire contents of which are hereby incorporated by reference herein). In MVM this splice has alternative donors (D1 and D2) and acceptors (A1 and A2) of different strengths, which are positioned within a region of 120 nt so that a potential D2: Al splice is eliminated by minimal intron size constraints. Splicing therefore creates 3 forms of each mRNA size class that are expressed with different stoichiometry (Haut and Pintel 1999, the entire contents of which are hereby incorporated by reference herein). Transcripts arising from P4 that have just this central intron removed encode a single form of NS1, translation of which terminates upstream of D1. In some P4 transcripts however, a second, long intron between 10-40 mu is also excised, creating mRNAs that encode NS2 proteins of ˜25 kDa. These share 85 amino acids of N-terminal sequence with NS1, but are then spliced into a different reading frame and finally reach a short central intron where 2 disparate C-terminal hexapeptides can be added. This generates variants called NS2P and NS2Y that are expressed in a ˜5:1 ratio. P38 transcription is strongly transactivated by the C-terminal domain of NS1, mediated by NS1 binding to upstream 5′-TGGT-3′ repeat sequences (Christensen et al., 1995, Lorson et al., 1996, the contents of which are hereby incorporated by reference herein in its entirety). Alternative splicing at a short intron also causes two size variants of a capsid polypeptide to be expressed with ˜1:5 stoichiometry, with VP1 (˜83 kDa) initiating at an ATG codon positioned between the two acceptor sites while VP2 (˜64 kDa) initiates downstream of the splice.

During infection, newly synthesized capsid polypeptides assemble as two types of trimers (VP2-only and 1×VP1+2×VP2) in the cytoplasm, and are transported into the nucleus for capsid-assembly using a non-conventional, structure-dependent trafficking motif (Lombardo et al., 2000). However, this translocation is restricted to S-phase (Gil-Ranedo et al., 2015, the contents of which are hereby incorporated by reference herein in its entirety), and is dependent upon trimer phosphorylation by the cellular Raf-1 kinase (Riolobos et al., 2010, the contents of which are hereby incorporated by reference herein in its entirety).

Ancillary polypeptides encoded by protoparvoviruses include the NS2 variants, which appear to have multiple functions that are mostly mediated by interactions with host proteins, and a small alternatively translated (SAT) protein (Zádori et al., 2005, the contents of which are hereby incorporated by reference herein in its entirety). MVM NS2 is not essential in transformed human cell lines, but its absence in murine cells leads to rapid cessation of duplex DNA amplification early in the infectious cycle by an unknown mechanism (Naeger et al., 1990, Ruiz et al., 2006, the contents of which are hereby incorporated by reference herein in its entirety). This early defect can be abrogated by relatively low levels of NS2 expression, but much higher levels of NS2 are required later in a cycle to enable efficient capsid assembly (Cotmore et al., 1997, the contents of which are hereby incorporated by reference herein in its entirety), which is a pre-requisite for the subsequent accumulation of progeny DNA single-strands, and for virion release. In a late capsid defect, VP polypeptides are expressed, but most fail to assemble into capsid polypeptides and are rapidly degraded, perhaps reflecting inadequacies in nuclear translocation of precursor subunits linked to a severe dislocation in normal nuclear/cytoplasmic protein trafficking, as discussed below. During MVM infection NS2 associates with proteins from a cellular 14-3-3 family (Brockhaus et al., 1996, the contents of which are hereby incorporated by reference herein in its entirety) and with the nuclear export factor CRM1 (Bodendorf et al., 1999, the contents of which are hereby incorporated by reference herein in its entirety). Significantly, a NS2 nuclear export signal (NES) engages CRM1 with “supraphysiological” affinity, which is independent of presence of RanGTP and thus can potentially resist cytoplasmic release (Engelsma et al., 2008, the contents of which are hereby incorporated by reference herein in its entirety). During wildtype MVM infection CRM1 can be detected in perinuclear cytoplasm, but this redistribution is exacerbated in infections with mutant viruses that carry point mutations close to the NS2 NES that cause CRM1 to bind at even higher affinity (López-Bueno et al., 2004, the contents of which are hereby incorporated by reference herein in its entirety). These mutations also accelerate onset of a late step in infection, which is characterized by a cytoplasmic accumulation of large, typically nuclear structures including NS1 and empty capsid polypeptides, again suggesting major disruptions in normal nuclear/cytoplasmic trafficking pathways. Following transfection into A9 fibroblasts, wildtype MVMi genomes express low levels of NS2, but when genomes were engineered to express one of a NS2-NES mutations, resulting low levels of mutant NS2 were able to drive wildtype levels of virus progeny accumulation, confirming that cumulative late infection blocks seen in cells expressing insufficient NS2 result from a stoichiometric limitation of NS2: CRM1 interactions (Choi et al., 2005, the contents of which are hereby incorporated by reference herein in its entirety). Studies with mutant viruses in which NS2: CRM1 binding was impaired, rather than enhanced, similarly indicate that during infection this interaction is required for the efficient release of virions (Eichwald et al., 2002, Miller and Pintel 2002, the contents of which are hereby incorporated by reference herein in its entirety).

A second protoparvovirus ancillary polypeptide, SAT, is encoded within a capsid gene and is expressed late, from the same mRNA as VP2. SAT accumulates in endoplasmic reticulum (ER) of a infected cell (Zádori et al., 2005, the contents of which are hereby incorporated by reference herein in its entirety). Like NS2, it enhances the rate at which virus spreads through cultures but it acts via a different mechanism that involves induction of irreversible ER-stress and is linked to enhanced cell necrosis (Mészáros et al., 2017b, the contents of which are hereby incorporated by reference herein in its entirety). Although both SAT and a dependoparvovirus ancillary polypeptide, AAP, occupy similar positions in a capsid gene and contain essential N-terminal hydrophobic domains, these polypeptides are not known to exhibit functional homology. Thus, in protoparvoviruses early virion export is a distinctive feature that can be driven by multiple mechanisms, either occurring prior to cell lysis and mediated by VP2 signals or Crm1 interactions that vary with cell type, or linked to enhanced cell necrosis and driven by SAT. During export, some virions can be internalized in COPII vesicles in a endoplasmic reticulum and undergo gelsolin-dependent trafficking to a Golgi, where they undergo tyrosine phosphorylation, and perhaps by other modifications that enhance their subsequent particle-to-infectivity ratios (Bär et al., 2008, Bär et al., 2013, the contents of which are hereby incorporated by reference herein in its entirety). Release at early times in a cycle allows infection to spread rapidly, potentially enhancing overall progeny production from infected tissues and prior to accumulation of neutralizing antibodies.

d. Exemplary Protoparvovirus

Among other things, the present disclosure provides exemplary protoparvovirus that can be used in accordance with embodiments described herein.

Exemplary Protoparvovirus species include human bufavirus genotypes 1, 2 and 3, human tusavirus, human cutavirus, canine parvovirus, porcine parvovirus, minute virus of mice and megabat bufavirus (see also Table 1 for nomenclature designated by International Committee on Taxonomy of Viruses (ICTV); world wide web at talk.ictvonline.org/taxonomy/, the entire contents of which are hereby incorporated by reference herein).

i. Kilham Rat Virus (KRV) and Minute Virus of Mice (MVM)

Kilham rat virus (KRV), one of the original viruses used to establish family Parvoviridae, was isolated in 1959 from lysates of an experimental rat tumor (Kilham and Olivier 1959, the contents of which are hereby incorporated by reference herein in its entirety). Over the next decade, a succession of similar single-stranded DNA viruses were discovered in transplantable tumors, tissue culture cell lines, or laboratory stocks of other viruses. Some of these, such as MVM, closely resemble viruses now known to infect wild rodents, while other members of the same species (Rodent protoparvovirus 1), such as LuIII (M81888), appear to be distant recombinants of viruses found in nature. Studied extensively in the intervening years, these viruses have served as important model systems for defining the basic characteristics and underlying biology of the family. In rodents, viruses from species Rodent protoparvovirus 1 exhibit a range of pathologies, from asymptomatic viremia to teratogenesis and fetal or neonatal cell death. While these viruses fail to infect normal human cells, host restrictions are often relaxed when human cells undergo oncogenic transformation, allowing viruses to become preferentially oncolytic, and suggesting their potential for use in clinical cancer virotherapy. To this end, Phase I/IIa clinical trials were recently completed using virus H-1 (X01457) to target advanced glioblastoma, which provided evidence that a virus was well tolerated and could partially disrupt the local immune suppression commonly associated with cancer (Geletneky et al., 2017, Angelova et al., 2017, the contents of which are hereby incorporated by reference herein in its entirety).

In some cells parvovirus infection results in delayed but significant type 1 IFN release, whereas pretreatment with exogenous IFN-beta strongly inhibits the viral life cycle (Grekova et al., 2010, Mattei et al., 2013, the contents of which are hereby incorporated by reference herein in its entirety). During MVMp infection of mouse embryonic fibroblasts (MEFs) the IFN response did not involve mitochondrial antiviral signaling protein (MAVS) and RIG-I sensing and did not conspicuously inhibit viral DNA replication (Mattei et al., 2013), although pretreatment of cells with IFN-beta-neutralizing antibody did enhance infection in another study (Grekova et al., 2010, the contents of which are hereby incorporated by reference herein in its entirety). However, infected MEFs become unresponsive to Poly (I: C) stimulation, suggesting that a virus is able to inactivate antiviral immune mechanisms elicited by type I IFNs.

ii. Feline Panleukopenia Virus (FPV)

Feline panleukopenia virus (FPV) is also known as feline parvovirus, and is closely related to mink and raccoon parvoviruses, which have existed for over 100 years, and canine parvovirus (CPV), which arose as a variant in the mid-1970s and in 1978 spread worldwide, causing a disease pandemic among dogs, wolves and coyotes. These variants all belong to a single species, Carnivore protoparvovirus 1. In adult animals, viruses in this species predominantly infect lymphoid tissues, leading to leukopenia or lymphopenia, and intestinal epithelia, resulting in severe diarrhea, dehydration and fever. In contrast, infection of neonates is characterized by cerebellar lesions in kittens or ferrets, potentially leading to ataxia, or by myocarditis in puppies. Disease is well controlled by vaccination, but mortality in affected litters varies between 20 and 100 percent (reviewed in (Kailasan et al., 2015a, the contents of which are hereby incorporated by reference herein in its entirety)).

iii. Porcine Parvovirus (PPV)

Porcine parvovirus (PPV), a member of the species Ungulate protoparvovirus 1, is a major cause of fetal death and infertility in pigs worldwide, although PPV infection alone rarely causes disease in non-pregnant pigs or piglets. However, when seronegative pregnant sows are exposed to a virulent PPV strain during first 70 days of gestation, transplacental infection can lead to a syndrome called SMEDI (stillbirths, mummification, embryonic death, and infertility) (Mészáros et al., 2017a, the contents of which are hereby incorporated by reference herein in its entirety). Weakly pathogenic and vaccine strains of PPV exist (e.g., NADL-2), which are lethal if injected into amniotic fluid but they do not cross a placental barrier as efficiently as pathogenic strains (e.g., Kresse), so disease is rare. Widespread vaccination programs are in place to prevent SMEDI, but some newly emerging virulent PPV variants cannot be neutralized by antibodies raised by exposure to current vaccine strains (Mészáros et al., 2017a, the contents of which are hereby incorporated by reference herein in its entirety). Co-infection with PPV can also potentiate the effect of porcine circovirus type 2 (PCV-2, Porcine circovirus 2, family Circoviridae) in the development of post-weaning multisystemic wasting syndrome (PMWS).

iv. Bufavirus (BuV)

Most newly discovered viruses segregate to species in a new branch of the Protoparvovirus tree, established for bufavirus 1a (human). Two genotypes of this virus, BuV1 and BuV2, were identified in 2012 in viral metagenomic analysis of fecal samples from diarrheic children in Burkina Faso and Tunisia (hence the name “bufavirus”) (Phan et al., 2012, the contents of which are hereby incorporated by reference herein in its entirety), while a third genotype, BuV3, was later discovered in the diarrheal feces of Bhutanese children (Yahiro et al., 2014, the contents of which are hereby incorporated by reference herein in its entirety). To date, BuV DNA has been detected in diarrhea of children from Burkina Faso, Tunisia, Bhutan, Thailand, Turkey, China, and Finland, and of adults from Finland, the Netherlands, Thailand, and China, but has not been found in non-diarrheal feces, suggesting a causal relationship (Väisänen et al., 2017, the contents of which are hereby incorporated by reference herein in its entirety). When analyzed for the presence of anti-BuVI capsid IgG, the seroprevalences of adults from Finland and the USA were low (˜2-4%), but much higher rates were found for adults in Iraq (˜85%), Iran (˜56%) and Kenya (˜72%) (Väisänen et al., 2018, the contents of which are hereby incorporated by reference herein in its entirety).

v. Cutavirus (CuV)

A second human protoparvovirus in a bufavirus branch, called cutavirus (CuV), was detected in a small number of diarrheal samples from Brazilian and Botswanan children, and in four French skin biopsies of cutaneous T-cell lymphomas, from which the virus derives its name (Phan et al., 2016, the contents of which are hereby incorporated by reference herein in its entirety), and in malignant skin lesions from a Danish melanoma patient (Mollerup et al., 2017). Etiological significance of CuV in human disease has yet to be determined.

Prevalence rates for IgG against CuV were evenly low (0-˜ 6%) in the same sample series mentioned above for bufavirus, confirming that CuV is widely distributed through human populations (Väisänen et al., 2018, the contents of which are hereby incorporated by reference herein in its entirety). In contrast, IgG directed against a third new, as yet unclassified protoparvovirus that was detected in a Tunisian human fecal sample (hence tusavirus, TuV) (Phan et al., 2014) was not present in the same panels of sera, and its DNA has yet to be detected in other fecal samples (Väisänen et al., 2017, Väisänen et al., 2018, the contents of which are hereby incorporated by reference herein in its entirety), so evidence for TuV being a human virus is thus, so far, insufficient. It segregates phylogenetically with viruses occupying the original branch of the protoparvovirus phylogenetic tree, discussed previously.

vi. Canine Parvovirus (CPV)

Canine parvovirus (CPV) is a well-studied species of protoparvovirus. CPV infects wild and domestic dogs. CPV has a genome size of ˜5.3 kb, 600 bp larger than AAV. The large genome makes CPV particularly attractive for the transfer of genes in human cells that cannot be accommodated in AAV derived vectors. Because CPV does not normally infect humans, there is no humoral immunity pre-existing against CPV in human population, i.e., humans are seronegative for CPV capsid antigens. This is in stark contrast to AAV; humans are seropositive for AAV capsid antigen such that presence of neutralizing AAV antibodies excludes a large percentage of patients eligible for AAV gene therapy. Therefore, a lack of neutralizing antibodies against CPV antigen in humans makes the CPV viral particles, or a virion comprising a capsid polypeptide of CPV or a variant thereof, particularly useful for highly potent gene therapy applications to prevent or treat different human genetic diseases that cannot be treated efficiently with AAV-derived vectors. Without wishing to be bound to any theory, CPV uses a canine transferrin receptor (TfR or CD71) as a cellular receptor to enter the cell, a protein expressed in the external membrane of a canine host cells (Goodman, Lyi et al. 2010). CPV also can interact with a human TfR counterpart and therefore internalize and transduce human cells. In addition, as described above, a VP2 capsid polypeptide of CPV can be engineered to comprise at least one sequence variation that alter tropism and the specificity/affinity of target cell interaction and eventually the efficiency of target cell transduction.

TABLE 1
Exemplary Isolates of Protoparvovirus

| | |
|---|---|
| Species of Protoparvovirus | Carnivore protoparvovirus |
| | Carnivore protoparvovirus 1 |
| | Chiropteran protoparvovirus 1 |
| | Eulipotyphla protoparvovirus 1 |
| | Primate protoparvovirus 1 |
| | Primate protoparvovirus 2 |
| | Primate protoparvovirus 3 |
| | Primate protoparvovirus 4 |
| | Rodent protoparvovirus 1 |
| | Rodent protoparvovirus 2 |
| | Rodent protoparvovirus 3 |
| | Ungulate protoparvovirus 1 |
| | Ungulate protoparvovirus 2 |

| Exemplary Viruses | Accession No. | Ref Seq No. |
|---|---|---|
| Sea otter parvovirus | KU561552 | NC_030837 |
| Canine parvovirus | M19296 | NC_001539 |
| Megabat bufavirus 1 | LC085675 | NC_029797 |
| Mpulungu (shrew) bufavirus | AB937988 | NC_026815 |
| Bufavirus 1a (human) | JX027296 | NC_038544 |
| Wuharv (rhesus) parvovirus 1 | JX627576 | NC_039049 |
| Cutavirus (human); | KT868811 | NC_039050 |
| Human Cutavirus 1 | | |
| Tusavirus; | KJ495710 | — |
| Human tusavirus | | |
| Minute virus of mice | J02275 | NC_001510 |
| Rat parvovirus 1 | AF036710 | NC_038545 |
| Rat bufavirus SY-2015 | KT716186 | NC_028650 |
| Porcine parvovirus; | L23427 | NC_001718 |
| Porcine parvovirus 5 | | |
| Porcine bufavirus; | KT965075 | NC_043446 |
| Protoparvovirus (porcine) | | |
| Porcine parvovirus 2 | — | NC_025965 |
| Porcine parvovirus 6 | — | NC_023860 |
| Feline panleukopeniavirus | FJ231389; | — |
| | KP769859 | |
| Human bufavirus 1 | JQ918261 | — |
| Human bufavirus 2 | JX027297 | — |
| Human bufavirus 3 | AB847989 | — |

e. Genotypic Variants of Viruses

An ordinarily skilled artisan appreciates that a species of virus comprises clusters of genetic variants (Van Regenmortel MHV (2000) Virus Taxonomy-Seventh Report of the International Committee on Taxonomy of Viruses). Genetic variants may comprise mutations (that encompasses point mutations and insertions-deletions of different lengths), hypermutations, several types of recombination, and genome segment reassortments. Mutation is observed in all viruses, with no known exceptions (Domingo (2019) Virus as Populations 2020:35-71). Recombination is also widespread, and its occurrence was soon accepted for DNA viruses as well as RNA viruses. Genome segment reassortment, a type of variation close to chromosomal exchanges in sexual reproduction, is an adaptive asset of segmented viral genomes, as continuously evidenced by the ongoing evolution of the influenza viruses. Three modes of virus genome variation are compatible, and reassortant-recombinant-mutant genomes are continuously arising in present-day viruses.

Accordingly, a genetic variant of viruses described herein may comprise a polypeptide described herein or those belonging to a virus or virion described herein (e.g., a capsid polypeptide (e.g., VP1 capsid polypeptide, VP2 capsid polypeptide, or variant thereof), NS1 polypeptide, etc.) with a polypeptide sequence that is at least, about, or no more than 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a polypeptide sequence of the exemplary sequences presented herein or a polypeptide sequence of the polypeptide of exemplary viruses referenced herein.

f. Marker and/or Reporter Genes

Exemplary marker genes include but not limited to any of fluorescent reporter genes, e.g., GFP, RFP and the like, as well as bioluminescence reporter genes. Exemplary marker genes include, but are not limited to, glutathione-S-transferase (GST), horseradish peroxidase (HRP), chloramphenicol acetyltransferase (CAT) beta-galactosidase, beta-glucuronidase, luciferase, green fluorescent proteins (e.g., GFP, GFP-2, tagGFP, turboGFP, sfGFP, EGFP, Emerald, Azami Green, Monomeric Azami Green, CopGFP, AceGFP, ZsGreenl), HcRed, DsRed, cyan fluo-rescent protein (CFP), yellow fluorescent proteins (e.g., YFP, EYFP, Citrine, Venus YPet, PhiYFP, ZsYellowl), cyan fluorescent proteins (e.g., ECFP, Cerulean, CyPet AmCyanl, Midoriishi-Cyan) red fluorescent proteins (e.g., mKate, mKate2, mPlum, DsRed monomer, mCherry, mRFPI, DsRed-Express, DsRed2, HcRed-Tandem, HcRed 1, AsRed2, eqFP6l 1, mRaspberry, mStrawberry, Jred), orange fluorescent proteins (e.g., mOrange, mKO, Kusabira-Orange, monomeric Kusabira-Orange, mTangerine, tdTomato) and autofluorescent proteins including blue fluorescent protein (BFP).

Marker genes may also include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where a marker sequence is the LacZ gene, a presence of a construct carrying a signal is detected by assays for β-galactosidase activity. In some embodiments, where a marker gene is green fluorescent protein or luciferase, a construct carrying a signal may be measured colorimetrically based on visible light absorbance or light production in a luminometer, respectively. Such reporters can, for example, be useful in verifying tissue-specific targeting capabilities and tissue specific promoter regulatory activity(ies) of a nucleic acid.

Marker genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate cellular metabolism resulting in enhanced cell growth rates and/or gene amplification (e.g., dihydrofolate reductase).

### 2. Compositions

Among other things, the present disclosure provides compositions. In some embodiments, a composition comprises a construct as described herein. In some embodiments, a composition comprises one or more constructs as described herein. In some embodiments, a composition comprises a plurality of constructs as described herein. In some embodiments, when more than one construct is included in the composition, the constructs are different from one another.

In some embodiments, a composition comprises a polynucleotide encoding a protoparvovirus variant VP1 capsid polypeptide. In some embodiments, a composition comprises a polynucleotide encoding a protoparvovirus VP2 capsid polypeptide.

In some embodiments, a composition comprises a virion as described herein. In some embodiments, a composition comprises one or more virions as described herein. In some embodiments, a composition comprises a plurality of virions. In some embodiments, when more than one type of virion is included in a composition, the more than one type of virions are each different types of virions.

In some embodiments, a composition comprises a cell. In some embodiments, a composition comprises a host cell. In some embodiments, a composition comprises an insect cell. In some embodiments, a composition comprises a mammalian cell. In some embodiments, a composition comprises a target cell.

In some embodiments, a composition is or comprises a pharmaceutical composition.

Among other things, in some embodiments, the present disclosure provides at least one sequence modification to a protoparvovirus VP1 capsid polypeptide that alters affinity and/or specificity of a virion to a cellular receptor involved in internalization of a virion, optionally wherein a cellular receptor is a transferrin receptor. In some embodiments, the at least one sequence modification of a protoparvovirus VP1 capsid polypeptide comprise: (a) at least one sequence variation that reduces toxicity of the virion in a host cell; (b) at least one sequence variation that increases virion production and/or virion production in a host cell; (c) at least one sequence variation that increases capsid polypeptide yield; or (d) any combination thereof.

In some embodiments, further provided herein is a virion comprising a protoparvovirus variant VP1 capsid polypeptide comprising a heterologous peptide tag. In some embodiments, a heterologous peptide tag allows affinity purification using an antibody, an antigen-binding fragment of an antibody, or a nanobody. In some embodiments, a heterologous peptide tag comprises an epitope/tag selected from hemagglutinin, His (e.g., 6X-His), FLAG, E-tag, TK15, Strep-tag II, AU1, AU5, Myc, Glu-Glu, KT3, and IRS.

Among other things, the present disclosure provides polynucleotides, e.g., polynucleotides comprising a VP1 capsid coding sequence operably linked to an expression control sequence, wherein the VP1 capsid coding sequence encodes a protoparvovirus variant VP1 capsid polypeptide. The present disclosure also provides methods utilizing such polynucleotides, e.g., in a composition (e.g., a pharmaceutical composition).

In some embodiments, a polynucleotide of the present disclosure may be or comprise DNA or RNA. In some embodiments, DNA can be genomic DNA or cDNA. In some embodiments, RNA can be an mRNA, an miRNA, a shRNA/siRNA, a gRNA, etc.

In some embodiments, a gene product is expressed from a polynucleotide comprising a VP1 capsid coding sequence operably linked to an expression control sequence, wherein the coding sequence encodes a protoparvovirus variant VP1 capsid polypeptide. In some embodiments, expression of such a polynucleotide can utilize one or more control elements (e.g., promoters, enhancers, splice sites, polyadenylation sites, translation initiation sites, etc.). Thus, in some embodiments, a polynucleotide provided herein can comprise one or more control elements.

In some embodiments, a VP1 gene is a protoparvovirus VP1 gene. In some embodiments, a protoparvovirus VP1 gene is a bufavirus VP1 gene as described herein. In some embodiments, a protoparvovirus VP1 gene is a canine parvovirus VP1 gene as described herein. In some embodiments, a protoparvovirus VP1 gene is a cutavirus VP1 gene as described herein. In some embodiments, a protoparvovirus VP1 gene is a feline panleukopenia VP1 gene as described herein. In some embodiments, a protoparvovirus VP1 gene is a minute virus of mice VP1 gene as described herein. In some embodiments, a protoparvovirus VP1 gene is a tusavirus VP1 gene described herein.

In some embodiments, a protoparvovirus VP1 capsid polypeptide is a bufavirus VP1 gene described herein. In some embodiments, a protoparvovirus VP1 capsid polypeptide is a canine parvovirus VP1 capsid polypeptide as described herein. In some embodiments, a protoparvovirus VP1 capsid polypeptide is a cutavirus VP1 capsid polypeptide as described herein. In some embodiments, a protoparvovirus VP1 capsid polypeptide is a feline panleukopenia VP1 capsid polypeptide as described herein. In some embodiments, a protoparvovirus VP1 capsid polypeptide is a minute virus of mice VP1 capsid polypeptide as described herein. In some embodiments, a protoparvovirus VP1 capsid polypeptide is a tusavirus VP1 capsid polypeptide as described herein.

Among other things, in some embodiments, the present disclosure describes exemplary constructs that have been engineered (e.g., see Exemplary Variant VP1 Capsid Sequences, see also, e.g., Table 4) to improve protoparvovirus VP1 capsid polypeptide production of a protoparvovirus VP1 capsid polypeptide in a host cell. Among other things, in some embodiments, the present disclosure describes exemplary constructs that have been engineered (e.g., see Exemplary Variant VP1 Capsid Sequences, see also, e.g., Table 4) to reduce toxicity of protoparvovirus VP1 capsid polypeptide in a host cell.

One skilled in the art would appreciate that a change (e.g., substitution, addition, deletion, etc.) of amino acids that are not conserved between a same polypeptide from different species is less likely to have an effect on the function of a protein and therefore, these amino acids should be selected for mutation. Amino acids that are conserved between a same polypeptide from different species should not be changed (e.g., deleted, added, substituted, etc.), as these mutations are more likely to result in a change in function of a polypeptide.

In some embodiments, a polynucleotide in accordance with the present disclosure comprises a protoparvovirus variant VP1 capsid polypeptide that is at least 85%, at least 90%, at least 95%, at least 98%, or at least 99% identical to a sequence of SEQ ID NOs: 103-110.

In some embodiments, a polypeptide provided herein comprises post-translational modifications. In some embodiments, a protoparvovirus variant VP1 capsid polypeptide provided herein comprises post-translational modifications. In some embodiments, post-translational modifications can comprise but is not limited to glycosylation (e.g., N-linked glycosylation, O-linked glycosylation), phosphorylation, acetylation, amidation, hydroxylation, methylation, ubiquitylation, sulfation, and/or a combination thereof.

a. Constructs

Among other things, the present disclosure provides that some polynucleotides as described herein are polynucleotide constructs. Polynucleotide constructs according to the present disclosure include all those known in the art, including cosmids, plasmids (e.g., naked or contained in liposomes) and constructs (e.g., protoparvovirus-related constructs) that incorporate a polynucleotide comprising a VP1 capsid coding sequence operably linked to an expression control sequence, wherein the VP1 capsid coding sequence encodes a protoparvovirus variant VP1 capsid polypeptide. Those of skill in the art will be capable of selecting suitable constructs, as well as cells, for making any of a nucleic acids described herein. In some embodiments, a construct is a plasmid (i.e., a circular DNA molecule that can autonomously replicate inside a cell). In some embodiments, a construct can be a cosmid (e.g., pWE or sCos series).

Constructs provided herein can be of different sizes. In some embodiments, a construct is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, or up to about 15 kb. In some embodiments, a construct is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb.

In some embodiments, a construct is a viral construct and can have a total number of nucleotides of up to 10 kb. In some embodiments, a viral construct can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some embodiments, a construct is a protoparvovirus construct and can have a total number of nucleotides of up to 6 kb in a single construct. In some embodiments, a construct can have a total number of nucleotides in the range of about 1 kb to about 2 kb, 1 kb to about 3 kb, about 1 kb to about 4 kb, about 1 kb to about 6 kb, about 2 kb to about 3 kb, about 2 kb to about 4 kb, about 2 kb to about 5 kb, about 3 kb to about 4 kb, about 3 kb to about 6 kb, about 4 kb to about 6 kb.

Any of constructs described herein can further include a control sequence, e.g., a control sequence selected from the group of a transcription initiation sequence, a transcription termination sequence, a promoter sequence, an enhancer sequence, an RNA splicing sequence, a polyadenylation (polyA) sequence, a Kozak consensus sequence, and/or additional untranslated regions which may house pre- or post-transcriptional regulatory and/or control elements. In some embodiments, a promoter can be a native promoter, a constitutive promoter, an inducible promoter, and/or a tissue-specific promoter. Non-limiting examples of control sequences are described herein. The foregoing methods for producing recombinant constructs are not meant to be limiting, and other suitable methods will be apparent to the skilled artisan.

b. Capsid Modifications

Among other things, the present disclosure describes insertion of one or more heterologous peptides into one or more residues of a protoparvovirus VP1 capsid polypeptide, or variant thereof, as described herein. In some embodiments, insertion of one or more heterologous peptides is at one or more residues of a protoparvovirus VP1 capsid polypeptide that map(s) onto a structural overlay of one or more residues within a variable region (e.g., VR (e.g., VR-IV, VR-V, VR-VIII)) of a parvovirus VP1 capsid (e.g., AAV capsid, e.g., AAV2 capsid, e.g., AAV5 capsid, e.g., AAV8 capsid, e.g., AAV9 capsid, or any variant thereof). In some embodiments, a heterologous peptide comprises or is a heterologous targeting peptide.

AAV VRs differ between serotypes and are responsible for serotype-specific variations in antibody and receptor binding (see Tseng and Agbandje-McKenna, 2014, the entire contents of which are hereby incorporated by reference herein). In some embodiments, one or more heterologous peptides increases cell specificity and/or viral transduction efficiency and/or increases virion performance of a protoparvovirus variant VP1 capsid polypeptide.

Adenovirus capsid modifications are described by Buning and Srivastava, 2019, the entire contents of which are hereby incorporated by reference herein. It is an insight of the present disclosure that, in some embodiments, one or more modifications introduced into one or more residues of an AAV capsid can be introduced into one or more corresponding residues of a variant VP1 protoparvovirus, as described herein. In some embodiments, one or more modifications described by Buning and Srivastava, 2019 are introduced into one or more residues of a protoparvovirus variant VP1 capsid polypeptide.

Among other things, the present disclosure describes insertion of one or more heterologous peptides into one or more residues along a 3-fold axis of symmetry of a protoparvovirus variant VP1 capsid polypeptide. Residues in regions along a 3-fold axis of symmetry of a capsid can be responsible for serotype-specific variations in antibody and/or receptor binding (see, Callaway et al., 2017, the entire contents of which are hereby incorporated by reference herein).

It is also an insight of the present disclosure that one or more modifications at one or more residues along a 3-fold axis of symmetry of a protoparvovirus VP1 capsid polypeptide, or variant thereof, can help re-direct or expand tropism (e.g., cell surface targeting) of viral-based gene therapies described herein.

Adenovirus capsid modifications are described by Buning and Srivastava, 2019, the entire contents of which are hereby incorporated by reference herein. It is an insight of the present disclosure that, in some embodiments, one or more modifications introduced in a variable region of an AAV capsid can be introduced into one or more residues along the 3-fold axis of symmetry of a variant VP1 protoparvovirus, as described herein. In some embodiments, one or more modifications described by Buning and Srivastava, 2019 are introduced into corresponding residues (e.g., along a 3-fold axis of symmetry) of a protoparvovirus VP1 capsid polypeptide. In some embodiments, one or more modifications are introduced into one or more residues along the 3-fold axis of symmetry of a protoparvovirus VP1 capsid polypeptide. In some embodiments a capsid modification is a peptide insertion. In some embodiments a capsid modification is a peptide insertion into a residue of a protoparvovirus VP1 capsid polypeptide that corresponds to a residue described by Buning and Srivastava, 2019. In some embodiments, one or more heterologous peptides is inserted into one or more residues along the 3-fold axis of symmetry of a common VP1 region of a protoparvovirus VP3 capsid polypeptide. In some embodiments, one or more heterologous peptides is inserted into one or more residues along a 3-fold axis of symmetry of a common VP2 region of a protoparvovirus VP1 capsid polypeptide.

In some embodiments, a heterologous peptide is inserted into one or more residues of a protoparvovirus variant VP1 capsid polypeptide corresponding to residue 587 of a common VP3 region of AAV2. In some embodiments, a heterologous peptide is inserted into one or more residues of a protoparvovirus variant VP1 capsid polypeptide corresponding to residue 588 of a common VP3 Region of AAV2. In some embodiments, a heterologous peptide is inserted into one or more residues of a protoparvovirus variant VP1 capsid polypeptide corresponding to residues other than 587 or 588 of a common VP3 region of AAV2. For example, in some embodiments, a heterologous peptide is inserted into one or more residues of a protoparvovirus variant VP1 capsid polypeptide corresponding to residue 453 of a common VP3 region of AAV2. In some embodiments, a heterologous peptide is inserted into one or more residues of a protoparvovirus variant VP1 capsid polypeptide corresponding to residue 585 of a common VP3 Region of AAV2. In some embodiments, a heterologous peptide is inserted into one or more residues of a protoparvovirus variant VP1 capsid polypeptide corresponding to residue 520 of a common VP3 Region of AAV2. In some embodiments, a heterologous peptide is inserted into one or more residues of a protoparvovirus variant VP1 capsid polypeptide corresponding to residue 584 of a common VP3 Region of AAV2.

In some embodiments, a heterologous peptide is inserted into one or more residues of a protoparvovirus variant VP1 capsid polypeptide corresponding to a common VP3 region of AAV1. For example, in some embodiments, a heterologous peptide is inserted into one or more residues of a protoparvovirus variant VP1 capsid polypeptide corresponding to residue 590 of a common VP3 Region of AAV1.

In some embodiments, a heterologous peptide is inserted into one or more residues of a protoparvovirus variant VP1 capsid polypeptide corresponding to a common VP3 Region of AAV3. For example, in some embodiments, a heterologous peptide is inserted into one or more residues of a protoparvovirus variant VP1 capsid polypeptide corresponding to residue 586 of a common VP3 Region of AAV3.

In some embodiments, a heterologous peptide is inserted into one or more residues of a protoparvovirus variant VP1 capsid polypeptide corresponding to a common VP3

Region of AAV4. For example, in some embodiments, a heterologous peptide is inserted into one or more residues of a protoparvovirus variant VP1 capsid polypeptide corresponding to residue 586 of a common VP3 Region of AAV4.

In some embodiments, a heterologous peptide is inserted into one or more residues of a protoparvovirus variant VP1 capsid polypeptide corresponding to a common VP3 Region of AAV5. For example, in some embodiments, a heterologous peptide is inserted into one or more residues of a protoparvovirus variant VP1 capsid polypeptide corresponding to residue 575 of a common VP3 Region of AAV5.

In some embodiments, a heterologous peptide is inserted into one or more residues of a protoparvovirus variant VP1 capsid polypeptide corresponding to a common VP3 Region of AAV6. For example, in some embodiments, a heterologous peptide is inserted into one or more residues of a protoparvovirus variant VP1 capsid polypeptide corresponding to residue 585 of a common VP3 Region of AAV6. In some embodiments, a heterologous peptide is inserted into one or more residues of a protoparvovirus variant VP1 capsid polypeptide corresponding to residue 585 in combination with mutation of a tryrosine to phenylalanine at residues 705 and 731 and mutation of threonine to valine at residue 492 of a common VP3 Region of AAV6. In some embodiments, a heterologous peptide is inserted into one or more residues of a protoparvovirus variant VP1 capsid polypeptide corresponding to residue 585 in combination with mutation of a tryrosine to phenylalanine at residues 705 and 731 and mutation of threonine to valine at residue 492 and mutation of lysine to glutamic acid at residue 531 of a common VP3 Region of AAV6.

In some embodiments, a heterologous peptide is inserted into one or more residues of a protoparvovirus variant VP1 capsid polypeptide corresponding to a common VP3 Region of AAV8. For example, in some embodiments, a heterologous peptide is inserted into one or more residues of a protoparvovirus variant VP1 capsid polypeptide corresponding to residue 585 of a common VP3 Region of AAV8. In some embodiments, a heterologous peptide is inserted into one or more residues of a protoparvovirus variant VP1 capsid polypeptide corresponding to residue 590 of a common VP3 Region of AAV8.

In some embodiments, a heterologous peptide is inserted into one or more residues of a protoparvovirus variant VP1 capsid polypeptide corresponding to a common VP3 Region of AAV9. For example, in some embodiments, a heterologous peptide is inserted into one or more residues of a protoparvovirus variant VP1 capsid polypeptide corresponding to residue 588 of a common VP3 Region of AAV9. In some embodiments, a heterologous peptide is inserted into one or more residues of a protoparvovirus variant VP1 capsid polypeptide corresponding to residue 589 of a common VP3 Region of AAV9.

In some embodiments, a heterologous peptide is inserted into one or more residues of a protoparvovirus variant VP1 capsid polypeptide corresponding to a common VP3 Region of AAV9P1.

In some embodiments, a heterologous peptide is inserted into one or more residues of a protoparvovirus variant VP1 capsid polypeptide corresponding to a common VP3 Region of AAV-PHP.B. For example, in some embodiments, a heterologous peptide is inserted into one or more residues of a protoparvovirus variant VP1 capsid polypeptide corresponding to residue 588 of a common VP3 Region of AAV-PHP.B. In some embodiments, a heterologous peptide is inserted into one or more residues of a protoparvovirus variant VP1 capsid polypeptide corresponding to residue 589 of a common VP3 Region of AAV-PHP.B.

Table 2 shows exemplary heterologous peptide sequences that can be inserted into one or more residues of a protoparvovirus variant VP1 capsid polypeptide described herein.

TABLE 2

| Exemplary Sequence Name | Amino Acid Sequence | SEQ ID NO: |
|---|---|---|
| Exemplary Heterologous Peptide 1 | QAGTFALRGDNPQG | SEQ ID NO: 5 |
| Exemplary Heterologous Peptide 2 | NGRAHA | SEQ ID NO: 6 |
| Exemplary Heterologous Peptide 3 | RGDAVGV | SEQ ID NO: 7 |
| Exemplary Heterologous Peptide 4 | RGDTPTS | SEQ ID NO: 8 |
| Exemplary Heterologous Peptide 5 | GENQARS | SEQ ID NO: 9 |
| Exemplary Heterologous Peptide 6 | RSNAVVP | SEQ ID NO: 10 |
| Exemplary Heterologous Peptide 7 | CDCRGDCFC | SEQ ID NO: 11 |
| Exemplary Heterologous Peptide 8 | PRGTNGP | SEQ ID NO: 12 |
| Exemplary Heterologous Peptide 9 | SRGATTT | SEQ ID NO: 13 |
| Exemplary Heterologous Peptide 10 | SIGYPLP | SEQ ID NO: 14 |
| Exemplary Heterologous Peptide 11 | MTPFPTSNEANL | SEQ ID NO: 15 |
| Exemplary Heterologous Peptide 12 | QPEHSST | SEQ ID NO: 16 |
| Exemplary Heterologous Peptide 13 | VNTANST | SEQ ID NO: 17 |
| Exemplary Heterologous Peptide 14 | CNHRYMQMC | SEQ ID NO: 18 |
| Exemplary Heterologous Peptide 15 | CAPGPSKSG | SEQ ID NO: 19 |
| Exemplary Heterologous Peptide 16 | EYHHYNK | SEQ ID NO: 20 |
| Exemplary Heterologous Peptide 17 | ASSLNIA | SEQ ID NO: 21 |
| Exemplary Heterologous Peptide 18 | TQVGQKT | SEQ ID NO: 22 |
| Exemplary Heterologous Peptide 19 | LPSSLQK | SEQ ID NO: 23 |
| Exemplary Heterologous Peptide 20 | WPFYGTP | SEQ ID NO: 24 |
| Exemplary Heterologous Peptide 21 | DSPAHPS | SEQ ID NO: 25 |
| Exemplary Heterologous Peptide 22 | GWTLHNK | SEQ ID NO: 26 |
| Exemplary Heterologous Peptide 23 | GMNAFRA | SEQ ID NO: 27 |
| Exemplary Heterologous Peptide 24 | LGETTRP | SEQ ID NO: 28 |
| Exemplary Heterologous Peptide 25 | RGDTATL | SEQ ID NO: 29 |
| Exemplary Heterologous Peptide 26 | PRGDLAP | SEQ ID NO: 30 |
| Exemplary Heterologous Peptide 27 | RGDQQSL | SEQ ID NO: 31 |
| Exemplary Heterologous Peptide 28 | EQLSISEEDL | SEQ ID NO: 32 |
| Exemplary Heterologous Peptide 29 | FNMQCQRRFYEALHDP | SEQ ID NO: 33 |
| | NLNEEQRNAKIKSIRDD | |
| | CX | |
| Exemplary Heterologous Peptide 30 | GLNDIFEAQKIEWHE | SEQ ID NO: 34 |
| Exemplary Heterologous Peptide 31 | LCTPSRAALLTGR | SEQ ID NO: 35 |
| Exemplary Heterologous Peptide 32 | QVSHWVSGLAEGSFG | SEQ ID NO: 36 |
| Exemplary Heterologous Peptide 33 | LSHTSGRVEGSVSLL | SEQ ID NO: 37 |
| Exemplary Heterologous Peptide 34 | VTAGRAP | SEQ ID NO: 38 |
| Exemplary Heterologous Peptide 35 | APVTRPA | SEQ ID NO: 39 |
| Exemplary Heterologous Peptide 36 | DLSNLTR | SEQ ID NO: 40 |
| Exemplary Heterologous Peptide 37 | NQVGSWS | SEQ ID NO: 41 |
| Exemplary Heterologous Peptide 38 | EARVRPP | SEQ ID NO: 42 |
| Exemplary Heterologous Peptide 39 | NSVSLYT | SEQ ID NO: 43 |
| Exemplary Heterologous Peptide 40 | NDVRSAN | SEQ ID NO: 44 |
| Exemplary Heterologous Peptide 41 | NESRVLS | SEQ ID NO: 45 |
| Exemplary Heterologous Peptide 42 | NRTWEQQ | SEQ ID NO: 46 |
| Exemplary Heterologous Peptide 43 | NSVQSSW | SEQ ID NO: 47 |
| Exemplary Heterologous Peptide 44 | RGDLGLS | SEQ ID NO: 48 |
| Exemplary Heterologous Peptide 45 | RGDMSRE | SEQ ID NO: 49 |
| Exemplary Heterologous Peptide 46 | ESGLSQS | SEQ ID NO: 50 |
| Exemplary Heterologous Peptide 47 | EYRDSSG | SEQ ID NO: 51 |
| Exemplary Heterologous Peptide 48 | DLGSARA | SEQ ID NO: 52 |
| Exemplary Heterologous Peptide 49 | GPQGKNS | SEQ ID NO: 53 |
| Exemplary Heterologous Peptide 50 | NSSRDLG | SEQ ID NO: 54 |
| Exemplary Heterologous Peptide 51 | NDVRAVS | SEQ ID NO: 55 |
| Exemplary Heterologous Peptide 52 | PRSTSDP | SEQ ID NO: 56 |
| Exemplary Heterologous Peptide 53 | DIIRA | SEQ ID NO: 57 |
| Exemplary Heterologous Peptide 54 | SYENVASRRPEG | SEQ ID NO: 58 |
| Exemplary Heterologous Peptide 55 | PENSVRRYGLEE | SEQ ID NO: 59 |
| Exemplary Heterologous Peptide 56 | LSLASNRPTATS | SEQ ID NO: 60 |
| Exemplary Heterologous Peptide 57 | NDVWNRDNSSKRGGTT | SEQ ID NO: 61 |
| | EAS | |
| Exemplary Heterologous Peptide 58 | NRTYSSTSNSTSRSEWD | SEQ ID NO: 62 |
| | NS | |
| Exemplary Heterologous Peptide 59 | ESGHGYF | SEQ ID NO: 63 |
| Exemplary Heterologous Peptide 60 | GQHPRPG | SEQ ID NO: 64 |
| Exemplary Heterologous Peptide 61 | PSVSPRP | SEQ ID NO: 65 |
| Exemplary Heterologous Peptide 62 | VNSTRLP | SEQ ID NO: 66 |
| Exemplary Heterologous Peptide 63 | LSPVRPG | SEQ ID NO: 67 |
| Exemplary Heterologous Peptide 64 | MSSDPRRPPRDG | SEQ ID NO: 68 |
| Exemplary Heterologous Peptide 65 | GARPSEVTTRPG | SEQ ID NO: 69 |
| Exemplary Heterologous Peptide 66 | GNEVLGTKPRAP | SEQ ID NO: 70 |
| Exemplary Heterologous Peptide 67 | KMRPGAMGTTGEGTRV | SEQ ID NO: 71 |
| | TRE | |
| Exemplary Heterologous Peptide 68 | MNVRGDL | SEQ ID NO: 72 |
| Exemplary Heterologous Peptide 69 | ENVRGDL | SEQ ID NO: 73 |
| Exemplary Heterologous Peptide 70 | KTLLPTP | SEQ ID NO: 74 |
| Exemplary Heterologous Peptide 71 | HLNILSTLWKYR | SEQ ID NO: 75 |
| Exemplary Heterologous Peptide 72 | SKAGRSP | SEQ ID NO: 76 |
| Exemplary Heterologous Peptide 73 | RGD | SEQ ID NO: 77 |
| Exemplary Heterologous Peptide 74 | PERTAMSLP | SEQ ID NO: 78 |
| Exemplary Heterologous Peptide 75 | ESGLSQS | SEQ ID NO: 79 |
| Exemplary Heterologous Peptide 76 | SEGLKNL | SEQ ID NO: 80 |
| Exemplary Heterologous Peptide 77 | SLRSPPS | SEQ ID NO: 81 |
| Exemplary Heterologous Peptide 78 | RGDLRVS | SEQ ID NO: 82 |
| Exemplary Heterologous Peptide 79 | TLAVPFK | SEQ ID NO: 83 |
| Exemplary Heterologous Peptide 80 | YTLSQGW | SEQ ID NO: 84 |

Among other things, in some embodiments, the present disclosure describes compositions, preparations, constructs, virions, population of virions, and host cells comprising a coding sequence that encodes a protoparvovirus variant VP1 capsid polypeptide further comprise an insertion of one or more heterologous peptides as described by Borner et al., 2020, the contents of which are hereby incorporated by reference in its entirety. In some embodiments, a heterologous peptide comprises a length of from 10 amino acids to 20 amino acids. In some embodiments, an insertion of one or more heterologous peptides is at one or more residues along a 3-fold axis of symmetry of a VP1 capsid polypeptide. In some embodiments, a protoparvovirus variant VP1 capsid polypeptide confers increased infectivity compared to the infectivity by a reference virion comprising the corresponding protoparvovirus reference VP1 capsid polypeptide. In some embodiments, the heterologous peptide alters cell specificity and/or viral transduction efficiency. In some embodiments the heterologous peptide increases virion performance.

In some embodiments, a protoparvovirus variant VP1 capsid polypeptide comprises a threonine to serine mutation at a residue corresponding to residue 590 of a HBoV reference VP1 capsid polypeptide (SEQ ID NO: 85), relative to a protoparvovirus reference VP1 capsid polypeptide. In some embodiments, a protoparvovirus variant VP1 capsid polypeptide comprises an aspartic acid to asparagine mutation at a residue corresponding to residue 86 of a HBoV reference VP1 capsid polypeptide (SEQ ID NO: 85), relative to a protoparvovirus reference VP1 capsid polypeptide. In some embodiments, a protoparvovirus variant VP1 capsid polypeptide comprises a serine to asparagine mutation at a residue corresponding to residue 474 of a HBoV reference VP1 capsid polypeptide (SEQ ID NO: 85), relative to a protoparvovirus reference VP1 capsid polypeptide. In some embodiments, a protoparvovirus variant VP1 capsid polypeptide comprises an alanine to threonine mutation at a residue corresponding to residue 149 of a HBoV reference VP1 capsid polypeptide (SEQ ID NO: 85), relative to a protoparvovirus reference VP1 capsid polypeptide. In some embodiments, a protoparvovirus variant VP1 capsid polypeptide comprises a threonine to serine mutation at a residue corresponding to residue 590, an aspartic acid to asparagine mutation at a residue corresponding to residue 86, a serine to asparagine mutation at a residue corresponding to residue 474, an alanine to threonine mutation at a residue corresponding to residue 149, or any combination thereof, of a HBoV reference VP1 capsid polypeptide (SEQ ID NO: 85), relative to a protoparvovirus reference VP1 capsid polypeptide.

Exemplary HBOV reference VP1 capsid polypeptide
(SEQ ID NO: 85)
MPPIKRQPRGWVLPGYRYLGPFNPLDNGEPVNNADRAAQLHDHAYSELI
KSGKNPYLYFNKADEKFIDDLKDDWSIGGIIGSSFFKIKRAVAPALGNK
ERAQKRHFYFANSNKGAKKTKKSEPKPGTSKMSDTDIQDQQPDTVDAPQ
NASGGGTGSIGGGKGSGVGISTGGWVGGSHFSDKYVVTKNTRQFITTIQ
NGHLYKTEAIETTNQSGKSQRCVTTPWTYFNFNQYSCHFSPQDWQRLTN
EYKRFRPKAMQVKIYNLQIKQILSNGADTTYNNDLTAGVHIFCDGEHAY
PNASHPWDEDVMPDLPYKTWKLFQYGYIPIENELADLDGNAAGGNATEK
ALLYQMPFFLLENSDHQVLRTGESTEFTFNFDCEWVNNERAYIPPGLMF
NPKVPTRRVQYIRQNGSTAASTGRIQPYSKPTSWMTGPGLLSAQRVGPQ
SSDTAPFMVCTNPEGTHINTGAAGFGSGFDPPSGCLAPTNLEYKLQWYQ
TPEGTGNNGNIIANPSLSMLRDQLLYKGNQTTYNLVGDIWMFPNQVWDR
FPITRENPIWCKKPRADKHTIMDPFDGSIAMDHPPGTIFIKMAKIPVPT
ATNADSYLNIYCTGQVSCEIVWEVERYATKNWRPERRHTALGMSLGGES
NYTPTYHVDPTGAYIQPTSYDQCMPVKTNINKVL

c. Exemplary Capsid Construct Sequences

The present disclosure provides technologies (e.g., compositions, methods, etc.) that are or comprise constructs described herein. In some embodiments, technologies described herein comprise a protoparvovirus variant VP1 capsid polypeptide. In some embodiments, technologies comprising a protoparvovirus variant VP1 capsid polypeptide result in improved characteristics compared to technologies comprising a protoparvovirus reference VP1 capsid polypeptide as described herein.

Among other things, in some embodiments, constructs described herein comprise a VP1 capsid coding sequence and a VP2 capsid coding sequence. In some embodiments, constructs describe herein further comprise a Rep sequence (e.g., AAV Rep protein).

i. Reference VP1 Capsid Sequences

In some embodiments, constructs, compositions, virions, or populations of virions comprise a parvovirus VP1 capsid polypeptide having a VP1 capsid coding sequence that shows at least 70%, at least 80%, at least 85%, at least 90%, at least 91%, at least 92%, at least 93%, at least 94%, at least 95%, at least 96%, at least 97%, at least 98%, at least 99%, at least 100% overall sequence identity with that of a parvovirus reference VP1 capsid selected from the group consisting of those in Table 3A.

Table 3A shows exemplary parvovirus reference VP1 capsid polypeptide sequences described herein.

TABLE 3A

| Reference | | | |
|---|---|---|---|
| Sequence | | | SEQ |
| Name | GenBank # | Sequence | ID NO: |
| Exemplary | AJ564427.2 | ATGGCACCTCCGGCAAAGAGAGCCAGGAGAGG | SEQ ID |
| Canine | | TAAGGGTGTGTTAGTAAAGTGGGGGGAGGGGA | NO: 86 |
| parvovirus | | AAGATTTAATAACTTAACTAAGTATGTGTTTT | |
| VP1 capsid | | TTTATAGGACTTGTGCCTCCAGGTTATAAATA | |
| coding | | TCTTGGGCCTGGGAACAGTCTTGACCAAGGAG | |
| sequence | | AACCAACTAACCCTTCTGACGCCGCTGCAAAA | |
| | | GAACACGACGAAGCTTACGCTGCTTATCTTCG | |
| | | CTCTGGTAAAAACCCATACTTATATTTCTCGC | |
| | | CAGCAGATCAACGCTTTATAGATCAAACTAAG | |
| | | GACGCTAAAGATTGGGGGGGGAAAATAGGACA | |
| | | TTATTTTTTTAGAGCTAAAAAGGCAATTGCTC | |
| | | CAGTATTAACTGATACACCAGATCATCCATCA | |
| | | ACATCAAGACCAACAAAACCAACTAAAAGAAG | |
| | | TAAACCACCACCTCATATTTTCATCAATCTTG | |
| | | CAAAAAAAAAAAAAGCCGGTGCAGGACAAGTA | |
| | | AAAAGAGACAATCTTGCACCAATGAGTGATGG | |
| | | AGCAGTTCAACCAGACGGTGGTCAGCCTGCTG | |
| | | TCAGAAATGAAAGAGCTACAGGATCTGGGAAC | |
| | | GGGTCTGGAGGCGGGGGTGGTGGTGGTTCTGG | |
| | | GGGTGTGGGGATTTCTACGGGTACTTTCAATA | |
| | | ATCAGACGGAATTTAAATTTTTGGAAAACGGA | |
| | | TGGGTGGAAATCACAGCAAACTCAAGCAGACT | |
| | | TGTACATTTAAATATGCCAGAAAGTGAAAATT | |
| | | ATAGAAGAGTGGTTGTAAATAATTTGGATAAA | |
| | | ACTGCAGTTAACGGAAACATGGCTTTAGATGA | |
| | | TACTCATGCACAAATTGTAACACCTTGGTCAT | |
| | | TGGTTGATGCAAATGCTTGGGGAGTTTGGTTT | |
| | | AATCCAGGAGATTGGCAACTAATTGTTAATAC | |
| | | TATGAGTGAGTTGCATTTAGTTAGTTTTGAAC | |
| | | AAGAAATTTTTAATGTTGTTTTAAAGACTGTT | |
| | | TCAGAGTCTGCTACTCAGCCACCAACAAAAGT | |
| | | TTATAATAATGATTTAACTGCATCATTGATGG | |
| | | TTGCATTAGATAGTAATAATACTATGCCATTT | |
| | | ACTCCAGCAGCTATGAGATCTGAGACATTGGG | |
| | | TTTTTATCCATGGAAACCAACCATACCAACTC | |
| | | CATGGAGATATTATTTTCAATGGGATAGAACA | |
| | | TTAATACCATCTCATACTGGAACTAGTGGCAC | |
| | | ACCAACAAATATATACCATGGTACAGATCCAG | |
| | | ATGACGTTCAATTTTATACTATTGAAAATTCT | |
| | | GTGCCAGTACACTTACTAAGAACAGGAGATGA | |
| | | ATTTGCTACAGGAACATTTTTTTTTGATTGTA | |
| | | AACCATGTAGACTAACACATACATGGCAAACA | |
| | | AATAGAGCATTGGGCTTACCACCATTTCTAAA | |
| | | TTCTTTGCCTCAAGCTGAAGGAGGTACTAACT | |
| | | TTGGTTATATAGGAGTTCAACAAGATAAAAGA | |
| | | CGTGGTGTAACTCAAATGGGAAATACAAACTA | |
| | | TATTACTGAAGCTACTATTATGAGACCAGCTG | |
| | | AGGTTGGTTATAGTGCACCATATTATTCTTTT | |
| | | GAGGCGTCTACACAAGGGCCATTTAAAACACC | |
| | | TATTGCAGCAGGACGGGGGGGAGCGCAAACAG | |
| | | ATGAAAATCAAGCAGCAGATGGTGATCCAAGA | |
| | | TATGCATTTGGTAGACAACATGGTCAAAAAAC | |
| | | TACCACAACAGGAGAAACACCTGAGAGATTTA | |
| | | CATATATAGCACATCAAGATACAGGAAGATAT | |
| | | CCAGAAGGAGATTGGATTCAAAATATTAACTT | |
| | | TAACCTTCCTGTAACAAATGATAATGTATTGC | |
| | | TACCAACAGATCCAATTGGAGGTAAAGCAGGA | |
| | | ATTAACTATACCAATATATTTAATACTTATGG | |
| | | TCCTTTAACTGCATTAAATAATGTACCACCAG | |
| | | TTTATCCAAATGGTCAAATTTGGGATAAAGAA | |
| | | TTTGATACTGATTTAAAACCAAGACTTCATGT | |
| | | AAATGCACCATTTGTTTGTCAAAATAATTGTC | |
| | | CTGGTCAATTATTTGTAAAAGTTGCGCCTAAT | |
| | | TTAACAAATGAATATGATCCTGATGCATCTGC | |
| | | TAATATGTCAAGAATTGTAACTTACTCAGATT | |
| | | TTTGGTGGAAAGGTAAATTAGTATTTAAAGCT | |
| | | AAACTAAGAGCCTCTCATACTTGGAATCCAAT | |
| | | TCAACAAATGAGTATTAATGTAGATAACCAAT | |
| | | TTAACTATGTACCAAGTAATATTGGAGGTATG | |
| | | AAAATTGTATATGAAAAATCTCAACTAGCACC | |
| | | TAGAAAATTATATTAA | |
| Exemplary | J02275.1 | ATGAGTGATGGCACCAGCCAACCTGACAGCGG | SEQ ID |
| Minute | | AAACGCTGTCCACTCAGCTGCAAGAGTTGAAC | NO: 87 |
| virus of | | GAGCAGCTGACGGCCCTGGAGGCTCTGGGGGT | |
| mince VP1 | | GGGGGCTCTGGCGGGGGTGGGGTTGGTGTTTC | |
| capsid | | TACTGGGTCTTATGATAATCAAACGCATTATA | |
| coding | | GATTCTTGGGTGACGGCTGGGTAGAAATTACT | |
| sequence | | GCACTAGCAACTAGACTAGTACATTTAAACAT | |
| | | GCCTAAATCAGAAAACTATTGCAGAATCAGAG | |
| | | TTCACAATACAACAGACACATCAGTCAAAGGC | |
| | | AACATGGCAAAAGATGATGCTCATGAGCAAAT | |
| | | TTGGACACCATGGAGCTTGGTGGATGCTAATG | |
| | | CTTGGGGAGTTTGGCTCCAGCCAAGTGACTGG | |
| | | CAATACATTTGCAACACCATGAGCCAGCTTAA | |
| | | CTTGGTATCACTTGATCAAGAAATATTCAATG | |
| | | TAGTGCTGAAAACTGTTACAGAGCAAGACTTA | |
| | | GGAGGTCAAGCTATAAAAATATACAACAATGA | |
| | | CCTTACAGCTTGCATGATGGTTGCAGTAGACT | |
| | | CAAACAACATTTTGCCATACACACCTGCAGCA | |
| | | AACTCAATGGAAACACTTGGTTTCTACCCCTG | |
| | | GAAACCAACCATAGCATCACCATACAGGTACT | |
| | | ATTTTTGCGTTGACAGAGATCTTTCAGTGACC | |
| | | TACGAAAATCAAGAAGGCACAGTTGAACATAA | |
| | | TGTGATGGGAACACCAAAAGGAATGAATTCTC | |
| | | AATTTTTTACCATTGAGAACACACAACAAATC | |
| | | ACATTGCTCAGAACAGGGGACGAATTTGCCAC | |
| | | AGGTACTTACTACTTTGACACAAATTCAGTTA | |
| | | AACTCACACACACGTGGCAAACCAACCGTCAA | |
| | | CTTGGACAGCCTCCACTGCTGTCAACCTTTCC | |
| | | TGAAGCTGACACTGATGCAGGTACACTTACTG | |
| | | CTCAAGGGAGCAGACATGGAACAACACAAATG | |
| | | GGGGTTAACTGGGTGAGTGAAGCAATCAGAAC | |
| | | CAGACCTGCTCAAGTAGGATTTTGTCAACCAC | |
| | | ACAATGACTTTGAAGCCAGCAGAGCTGGACCA | |
| | | TTTGCTGCCCCAAAAGTTCCAGCAGATATTAC | |
| | | TCAAGGAGTAGACAAAGAAGCCAATGGCAGTG | |
| | | TTAGATACAGTTATGGCAAACAGCATGGTGAA | |
| | | AATTGGGCTTCACATGGACCAGCACCAGAGCG | |
| | | CTACACATGGGATGAAACAAGCTTTGGTTCAG | |
| | | GTAGAGACACCAAAGATGGTTTTATTCAATCA | |
| | | GCACCACTAGTTGTTCCACCACCACTAAATGG | |
| | | CATTCTTACAAATGCAAACCCTATTGGGACTA | |
| | | AAAATGACATTCATTTTTCAAATGTTTTTAAC | |
| | | AGCTATGGTCCACTAACTGCATTTTCACACCC | |
| | | AAGTCCTGTATACCCTCAAGGACAAATATGGG | |
| | | ACAAAGAACTAGATCTTGAACACAAACCTAGA | |
| | | CTTCACATAACTGCTCCATTTGTTTGTAAAAA | |
| | | CAATGCACCTGGACAAATGTTGGTTAGATTAG | |
| | | GACCAAACCTAACTGACCAATATGATCCAAAC | |
| | | GGAGCCACACTTTCTAGAATTGTTACATACGG | |
| | | TACATTTTTCTGGAAAGGAAAACTAACCATGA | |
| | | GAGCAAAACTTAGAGCTAACACCACTTGGAAC | |
| | | CCAGTGTACCAAGTAAGTGCTGAAGACAATGG | |
| | | CAACTCATACATGAGTGTAACTAAATGGTTAC | |
| | | CAACTGCTACTGGAAACATGCAGTCTGTGCCG | |
| | | CTTATAACAAGACCTGTTGCTAGAAATACTTA | |
| | | CTAA | |

In some embodiments, constructs, compositions, virions, or populations of virions comprise a protoparvovirus variant VP1 capsid polypeptide having a polypeptide sequence that shows at least 70%, at least 80%, at least 85%, at least 90%, at least 91%, at least 92%, at least 93%, at least 94%, at least 95%, at least 96%, at least 97%, at least 98%, at least 99%, at least 100% overall sequence identity with that of a protoparvovirus reference VP1 capsid selected from the group consisting of those in Table 3B.

Table 3B shows exemplary protoparvovirus reference VP1 capsid polypeptide sequences described herein.

TABLE 3B

| Reference | | | |
|---|---|---|---|
| Sequence | | | SEQ ID |
| Name | GenBank # | Sequence | NO: |
| Exemplary | AFN44271 | MPAIRKARGWVPPGYNYLGPFNQDFSK | SEQ ID |
| Bufavirus VP1 | | KPTNPSDNAARKHDLEYNKLIKQGHNP | NO: 88 |
| polypeptide | | YWNYNHADEDFIKETDQATDWGGKFGN | |
| sequence | | FVFRAKRALAPELAPPAKKKTKTKHTE | |
| | | PEYSHKHIKAGTKRGKPFYLFVNLARK | |
| | | KARMTDTQDVSEQQSDQPSVASTSAKA | |
| | | GGGGGGGGSGVGHSTGNYNNRTEFYYH | |
| | | GDEVTIVCHSSRHIHLNMSESEEYKIY | |
| | | DTDRGPTFPTDQTLQGRDTINDSYHAQ | |
| | | VETPWFLINPNSWGTWMNPADFQQLTT | |
| | | TCREVTLEHLDQTLDNIVIKTVSKOGS | |
| | | GAEETTQYNNDLTALLQVALDKSNQLP | |
| | | WVADNMYLDSLGYIPWRPCKLKQYSYH | |
| | | VNFWNTIDIISGPQQNQWQQVKKEIKW | |
| | | DDLQFTPIETTTEIDLLRTGDSWTSGP | |
| | | YKFNTKPTQLSYHWQSTRHTGSVHPTE | |
| | | PPNAIGQQGRNIIDINGWQWGDRSNPM | |
| | | SAATRVSNFHIGYSWPEWRIHYGSGGP | |
| | | AINPGAPFSQAPWSTDPQVRLTQGASE | |
| | | KAIFDYNHGDDDPAHRDQWWQNNLPMT | |
| | | GQTDWAPKNAHQTNVSNNIPSRQEFWT | |
| | | QDYHNTFGPFTAVDDVGIQYPWGAIWT | |
| | | KTPDTTHKPMMSAHAPFICKDGPPGQL | |
| | | LVKLAPNYTENLQTDGLGNNRIVTYAT | |
| | | FWWTGKLVLKGKLRLPRQFNLYNLPGR | |
| | | PRGTEAKKFLPNEIGHFELPFMPGRCM | |
| | | PNYTI | |
| Exemplary | M19296.1 | MAPPAKRARRGKGVLVKWGEGKDLITX | SEQ ID |
| Canine | | LSMCFFIGLVPPGYKYLGPGNSLDQGE | NO: 89 |
| Parvovirus VP1 | | PTNPSDAAAKEHDEAYAAYLRSGKNPY | |
| polypeptide | | LYFSPADQRFIDQTKDAKDWGGKIGHY | |
| sequence | | FFRAKKAIAPVLTDTPDHPSTSRPTKP | |
| | | TKRSKPPPHIFINLAKKKKAGAGQVKR | |
| | | DNLAPMSDGAVQPDGGQPAVRNERATG | |
| | | SGNGSGGGGGGGSGGVGISTGTFNNQT | |
| | | EFKFLENGWVEITANSSRLVHLNMPES | |
| | | ENYRRVVVNNMDKTAVNGNMALDDIHA | |
| | | QIVTPWSLVDANAWGVWFNPGDWQLIV | |
| | | NTMSELHLVSFEQEIFNVVLKTVSESA | |
| | | TQPPTKVYNNDLTASLMVALDSNNTMP | |
| | | FTPAAMRSETLGFYPWKPTIPTPWRYY | |
| | | FQWDRTLIPSHTGTSGTPTNIYHGTDP | |
| | | DDVQFYTIENSVPVHLLRTGDEFATGT | |
| | | FFFDCKPCRLTHTWQTNRALGLPPFLN | |
| | | SLPQSEGATNFGDIGVQQDKRRGVTQM | |
| | | GNTNYITEATIMRPAEVGYSAPYYSFE | |
| | | ASTQGPFKTPIAAGRGGAQTYENQAAD | |
| | | GDPRYAFGRQHGQKTTTTGETPERFTY | |
| | | IAHQDTGRYPEGDWIQNINFNLPVTND | |
| | | NVLLPTDPIGGKTGINYTNIFNTYGPL | |
| | | TALNNVPPVYPNGQIWDKEFDTDLKPR | |
| | | LHVNAPFVCONNCPGQLFVKVAPNLTN | |
| | | EYDPDASANMSRIVTYSDFWWKGKLVF | |
| | | KAKLRASHTWNPIQQMSINVDNQFNYV | |
| | | PSNIGGMKIVYEKSQLAPRKLY | |
| Exemplary | AXQ00350 | MAPPAKRARRGLVPPGYKYLGPGNSLD | SEQ ID |
| Canine | | QGEPTNPSDAAAKEHDEAYAAYLRSGK | NO: 90 |
| Parvovirus VP1 | | NPYLYFSPADQRFIDQTKDAKDWGGKI | |
| polypeptide | | GHYFFRAKKAIAPVLTDTPDHPSTSRP | |
| sequence | | TKPTKRSKPPPHIFINLAKKKKAGAGQ | |
| | | VKRDNLAPMSDGGVQPDGGQPAVRNER | |
| | | ATGSGNGSGGGGGGGSGGVGISTGTFN | |
| | | NQTEFKFLENGWVEITANSSRLVHLNM | |
| | | PESENYRRVVVNNLDKTAVNGNMALDD | |
| | | THAQIVTPWSLVDANAWGVWFNPGDWQ | |
| | | LIVNTMSELHLVSFEQEIFNVVLKTVS | |
| | | ESATQPPTKVYNNDLTASLMVALDSNN | |
| | | TMPFTPAAMRSETLGFYPWKPTIPTPW | |
| | | RYYFQWDRTLIPSHTGTSGTPTNIYHG | |
| | | TDPDDVQFYTIENSVPVHLLRTGDEFA | |
| | | TGTFYFDCKPCRLTHTWQTNRALGLPP | |
| | | FLNSLPQAEGGTNFGYIGVQQDKRRGV | |
| | | TQMGNTNIITEATIMRPAEVGYSAPYY | |
| | | SFEASTQGPFKTPIAAGRGGAQTDENR | |
| | | AADGDPRYAFGRQHGQKTTTTGETPER | |
| | | FTYIAHQDTGRYPEGDWIQNINFNLPV | |
| | | TEDNVLLPTDPIGGKTGINYTNIFNTY | |
| | | GPLTALNNVPPVYPNGQIWDKEFDTDL | |
| | | KPRLHVNAPFVCONNCPGQLFVKVAPN | |
| | | LTNEYDPDASANMSRIVTYSDFWWKGK | |
| | | LVFKAKLRASHTWNPIQQMSINVDNQF | |
| | | NYVPSNIGGMKIVYEKSQLAPRKLY | |
| Exemplary | AQN78782.1 | MPAIRKARGWVPPGYNFLGPFNQDENK | SEQ ID |
| Cutavirus | | EPTNPSDNAAKQHDLEYNKLINQGHNP | NO: 91 |
| VP1u-VP2 | | YWYYNKADEDFIKATDQAPDWGGKFGN | |
| polypeptide | | FIFRAKKHIAPELAPPAKKKSKTKHPE | |
| sequence | | PEFSHKHIKPGTKRGKPFHIFVNLARK | |
| | | RARMSEPAENTNDQPNDSPVEQGAGQI | |
| | | GGGGGGGGSGVGHSTGDYNNRTEFIYH | |
| | | GDEVTIICHSTRLVHINMSDREDYIIY | |
| | | ETDRGQLFPTTQDLQGRDTLNDSYHAK | |
| | | VETPWKLLHANSWGCWFSPADFQQMIT | |
| | | TCRDIAPIQMHQKIENIVIKTVSKTGT | |
| | | GETETTNYNNDLTALLQIAQDNSNLLP | |
| | | WAADNFYIDSVGYVPWRACKLPTYCYH | |
| | | VDTWNTIDINQADAPNRWREIKKGIQW | |
| | | DNIQFTPLETMINIDLLRTGDAWQSGN | |
| | | YNFHTKPTNLAYHWQSQRHTGSCHPTV | |
| | | APLVERGQGTNIQSVNCWQWGDRNNPS | |
| | | SASTRVSNMHIGYSFPEWQIHYSTGGP | |
| | | VINPGSAFSQAPWGSTTEGTRLTQGAS | |
| | | EKAIYDWAHGDDQPGARETWWQNNQHV | |
| | | TGQTDWAPKNAHTSELNNNVPAATHFW | |
| | | KNSYHNTFSPFTAVDDHGPQYPWGAIW | |
| | | GKYPDTTHKPMMSAHAPFLLHGPPGQL | |
| | | FVKLAPNYTDTLDNGGVTHPRIVTYGT | |
| | | FWWSGKLIFKGKLRTPRQWNTYNLPSL | |
| | | DKRETMKNTVPNEVGHFELPYMPGRCL | |
| | | PNYTL | |
| Exemplary | YP_009508805 | MPAIRKARGWVPPGYNFLGPFNQDENK | SEQ ID |
| Cutavirus | | EPTNPSDNAAKQHDLEYNKLINQGHNP | NO: 92 |
| VP1u-VP2 | | YWYYNKADEDFIKATDQAPDWGGKFGN | |
| polypeptide | | FIFRAKKHIAPELAPPAKKKSKTKHSE | |
| sequence | | PEFSHKHIKPGTKRGKPFHIFVNLARK | |
| | | RARMSEPANDTNEQPDNSPVEQGAGQI | |
| | | GGGGGGGGSGVGHSTGDYNNRTEFIYH | |
| | | GDEVTIICHSTRLVHINMSDREDYIIY | |
| | | ETDRGPLFPTTQDLQGRDTLNDSYHAK | |
| | | VETPWKLLHANSWGCWFSPADFQQMIT | |
| | | TCRDIAPIKMHQKIENIVIKTVSKTGT | |
| | | GETETTNYNNDLTALLQIAQDNSNLLP | |
| | | WAADNFYIDSVGYVPWRACKLPTYCYH | |
| | | VDTWNTIDINQADTPNQWREIKKGIQW | |
| | | DNIQFTPLETMINIDLLRTGDAWESGN | |
| | | YNFHTKPTNLAYHWQSQRHTGSCHPTV | |
| | | APLVERGQGTNIQSVNCWQWGDRNNPS | |
| | | SASTRVSNIHIGYSFPEWQIHYSTGGP | |
| | | VINPGSAFSQAPWGSTTEGTRLTQGAS | |
| | | EKAIYDWSHGDDQPGARETWWQNNQHV | |
| | | TGQTDWAPKNAHTSELNNNVPAATHFW | |
| | | KNSYHNTFSPFTAVDDHGPQYPWGAIW | |
| | | GKYPDTTHKPMMSAHAPFLLHGPPGQL | |
| | | FVKLAPNYTDTLDNGGVTHPRIVTYGT | |
| | | FWWSGQLIFKGKLRTPRQWNTYNLPSL | |
| | | DKRETMKNTVPNEVGHFELPYMPGRCL | |
| | | PNYTL | |
| Exemplary | ACD37389.1 | MAPPAKRARRGLVPPGYKYLGPGNSLD | SEQ ID |
| Feline | | QGEPTNPSDAAAKEHDEAYAAYLRSGK | NO: 93 |
| Panleukopenia | | NPYLYFSPADQRFIDQTKDAKDWGGKI | |
| Virus VP1 | | GHYFFRAKKAIAPVLTDTPDHPSTSRP | |
| polypeptide | | TKPTKRSKPPPHIFINLAKKKKAGAGQ | |
| sequence | | VKRDNLAPMSDGAVQPDGGQPAVRNER | |
| | | ATGSGNGSGGGGGGGSGGVGISTGTFN | |
| | | NQTEFKFLENGWVEITANSSRLVHLNM | |
| | | PESENYKRVVVNNMDKTAVKGNMALDD | |
| | | IHVQIVTPWSLVDANAWGVWFNPGDWQ | |
| | | LIVNTMSELHLVSFEQEIFNVVLKTVS | |
| | | ESATQPPTKVYNNDLTASLMVALDSNN | |
| | | TMPFTPAAMRSETLGFYPWKPTIPTPW | |
| | | RYYFQWDRTLIPSHTGTSGTPTNVYHG | |
| | | TDPDDVQFYTIENSVPVHLLRTGDEFA | |
| | | TGTFFFDCKPCRLTHTWQTNRALGLPP | |
| | | FLNSLPQSEGATNYGDIGVQQDKRRGV | |
| | | TQMGNTDYITEATIMRPAEVGYSAPYY | |
| | | SFEASTQGPFKTPIAAGRGGAQTDENQ | |
| | | AADGDPRYAFGRQHGQKTTTTGETPER | |
| | | FTYIAHQDTGRYPEGDWIQNINFNLPV | |
| | | TNDNVLLPTDPIGGKTGINYTNIFNTY | |
| | | GPLTALNNVPPVYPNGQIWDKEFDTDL | |
| | | KPRLHVNAPFVCQNNCPGQLFVKVAPN | |
| | | LTNEYDPDASANMSRIVTYSDFWWKGK | |
| | | LVFKAKLRASHTWNPIQQMSINVDNQF | |
| | | NYVPNNIGAMKIVYEKSQLAPRKLY | |
| Exemplary | AKI88071 | MAPPAKRARRGLVPPGYKYLGPGNSLD | SEQ ID |
| Feline | | QGEPTNPSDAAAKEHDEAYAAYLRSGK | NO: 94 |
| Panleukopenia | | NPYLYFSPADQRFIDQTKDAKDWGGKI | |
| Virus VP1 | | GHYFFRAKKAIAPVLTDTPDHPSTSRP | |
| polypeptide | | TKPTKRSKPPPHIFINLAKKKKAGAGQ | |
| sequence | | VKRDNLAPMSDGAVQPDGGQPAVRNER | |
| | | ATGSGNGSGGGGGGGSGGVGISTGTFN | |
| | | NQTEFKFLENGWVEITANSSRLVHLNM | |
| | | PESENYKRVVVNNMDKTAVKGNMALDD | |
| | | THVQIVTPWSLVDANAWGVWFNPGDWQ | |
| | | LIVNTMSELHLVSFEQEIFNVVLKTVS | |
| | | ESATQPPTKVYNNDLTASLMVALDSNN | |
| | | TMPFTPAAMRSETLGFYPWKPTIPTPW | |
| | | RYYFQWDRTLIPSHTGTSGTPTNVYHG | |
| | | TDPDDVQFYTIENSVPVHLLRTGDEFA | |
| | | TGTFFFDCKPCRLTHTWQTNRALGLPP | |
| | | FLNSLPQSEGATNFGDIGVQQDKRRGV | |
| | | TQMGNTDYITEATIMRPAEVGYSAPYY | |
| | | SFEASTQGPFKTPIAAGRGGAQTDENQ | |
| | | AADGDPRYAFGRQHGQKTTTTGETPER | |
| | | FTYIAHQDTGRYPEGDWIQNINFNLPV | |
| | | TNDNVLLPTDPIGGKTGINYTNIFNTY | |
| | | GPLTALNNVPPVYPNGQIWDKEFDTDL | |
| | | KPRLHVNAPFVCQNNCPGQLFVKVAPN | |
| | | LTNEYDPDASANMSRIVTYSDFWWKGK | |
| | | LVFKAKLRASHTWNPIQQMSINVDNQF | |
| | | NYVPNNIGAMKIVYEKSQLAPRKLY | |
| Exemplary | J02275.1 | MAPPAKRAKRGWVPPGYKYLGPGNSLD | SEQ ID |
| Minute Virus | | QGEPTNPSDAAAKEHDEAYDQYIKSGK | NO: 95 |
| of Mice VP1 | | NPYLYFSAADQRFIDQTKDAKDWGGKV | |
| polypeptide | | GHYFFRTKRAFAPKLATDSEPGTSGVS | |
| sequence | | RAGKRTRPPAYIFINQARAKKKLTSSA | |
| | | AQQSSQTMSDGTSQPDSGNAVHSAARV | |
| | | ERAADGPGGSGGGGSGGGGVGVSTGSY | |
| | | DNQTHYRFLGDGWVEITALATRLVHLN | |
| | | MPKSENYCRIRVHNTTDTSVKGNMAKD | |
| | | DAHEQIWTPWSLVDANAWGVWLQPSDW | |
| | | QYICNTMSQLNLVSLDQEIFNVVLKTV | |
| | | TEQDLGGQAIKIYNNDLTACMMVAVDS | |
| | | NNILPYTPAANSMETLGFYPWKPTIAS | |
| | | PYRYYFCVDRDLSVTYENQEGTVEHNV | |
| | | MGTPKGMNSQFFTIENTQQITLLRTGD | |
| | | EFATGTYYFDTNSVKLTHTWQTNRQLG | |
| | | QPPLLSTFPEADTDAGTLTAQGSRHGT | |
| | | TQMGVNWVSEAIRTRPAQVGFCQPHND | |
| | | FEASRAGPFAAPKVPADITQGVDKEAN | |
| | | GSVRYSYGKQHGENWASHGPAPERYTW | |
| | | DETSFGSGRDTKDGFIQSAPLVVPPPL | |
| | | NGILTNANPIGTKNDIHFSNVFNSYGP | |
| | | LTAFSHPSPVYPQGQIWDKELDLEHKP | |
| | | RLHITAPFVCKNNAPGQMLVRLGPNLT | |
| | | DQYDPNGATLSRIVTYGTFFWKGKLTM | |
| | | RAKLRANTTWNPVYQVSAEDNGNSYMS | |
| | | VTKWLPTATGNMQSVPLITRPVARNTY | |
| Exemplary | AIT18930 | MAPAARPRKGWVPPGYNYLGPGNDLDA | SEQ ID |
| Tusavirus | | GEPTNKSDAAARKHDFAYSAYLKQGLD | NO: 96 |
| VP1 | | PYWNFNKADEKFIRDTEGATDWGGRLG | |
| polypeptide | | HWIFRAKKHILPHLKEPTLAGRKRPAP | |
| sequence | | AHIFVNLANKRKKGLPTRKDQQKDTLD | |
| | | SNAQQPVREADQPDGMAASSSDSGPSS | |
| | | SGGGARAGGVGVSTGDFDNTTLWDFHE | |
| | | DGTATITCNSTRLVHLTRPDSLDYKII | |
| | | PTQNNTAVQTVGHMMDDDNHTQVLTPW | |
| | | SLVDCNAWGVWLSPHDWQHIMNIGEEL | |
| | | ELLSLEQEVFNVTLKTATETGPPESRI | |
| | | TMYNNDLTAVMMITTDTNNQLPYTPAA | |
| | | IRSETLGFYPWRPTVVPRWRYYFDWDR | |
| | | FLSVTSSSDQSTSIINHSSTQSAIGQF | |
| | | FVIETQLPIALLRTGDSYATGGYKFDC | |
| | | NKVNLGRHWQTTRSLGLPPKIEPPTSE | |
| | | SALGTINQNARLGWRWGINDVHETNVV | |
| | | RPCTAGYNHPEWFYTHTLEGPAIDPAP | |
| | | PTSIPSNWGGGTPPDTRASSHNQQRIT | |
| | | YNYNHGNKDENLNNFSLNPNIELGSII | |
| | | NQGNFLSYEGNGQQINTTAGVGKNGET | |
| | | ATSDPNLVRYMPNTYGVYTAVDHQGPV | |
| | | YPHGQIWDKQIHTDKKPELHCLAPFTC | |
| | | KNNPPGQMFVRIAPNLTDTFNATPTFS | |
| | | EIITYADFWWKGTLKMKIKLRPPHQWN | |
| | | IATVLGAAVNIGDAARFVPNRLGQLEF | |
| | | PVINGRIVPSTVY | |

ii. Exemplary Variant VP 1 Capsid Sequences

In some embodiments, constructs, compositions, virions, or populations of virions comprise a VP1 capsid coding sequence that encodes a protoparvovirus variant VP1 capsid polypeptide. In some embodiments, a protoparvovirus variant VP1 capsid polypeptide is encoded by a VP1 capsid coding sequence with at least 85%, 90%, 95%, 98% or 99% sequence identity to a VP1 capsid coding sequence described herein. In some embodiments, a protoparvovirus variant VP1 capsid comprises a polypeptide with at least 85%, 90%, 95%, 98% or 99% sequence identity to a polypeptide of a sequence described herein. In some embodiments, constructs described herein comprise fewer ATG sequence(s) across the length of a VP1 capsid coding sequence (e.g., in frame or out of frame) that encodes a protoparvovirus variant VP1 capsid polypeptide. In some embodiments, constructs described herein comprise fewer ATG sequence(s) across the length of a VP1 capsid coding sequence (e.g., in frame or out of frame) that encodes a protoparvovirus variant VP1 capsid polypeptide due to a substitution in one or more of “ATG” relative to a protoparvovirus reference VP1 capsid coding sequence described herein. In some embodiments, constructs described herein comprise fewer ATG sequence(s) across the length of a VP1 capsid coding sequence (e.g., in frame or out of frame) that encodes a protoparvovirus variant VP1 capsid polypeptide due to a deletion in one or more of “ATG” relative to a protoparvovirus reference VP1 capsid coding sequence described herein. In some embodiments, constructs described herein comprise fewer “ATG” sequence(s) across the length of a VP1 capsid coding sequence (e.g., in frame or out of frame, e.g., at position −3 or +4 relative to the first position of a VP1 capsid coding sequence) that encodes a protoparvovirus variant VP1 capsid polypeptide due to a conservative amino acid substitution in one or more of “ATG” relative to a protoparvovirus reference VP1 capsid coding sequence described herein. In some embodiments, constructs described herein comprise fewer “ATG” sequence(s) across the length of a VP1 capsid coding sequence (e.g., in frame or out of frame, e.g., at position −3 or +4 relative to the first position of a VP1 capsid coding sequence) that encodes a protoparvovirus variant VP1 capsid polypeptide due to a conservative amino acid substitution of one or more nucleotides surrounding an “ATG” (e.g., a conservative amino acid substitution within a Kozak consensus sequence) relative to a protoparvovirus reference VP1 capsid coding sequence described herein. In some embodiments, constructs described herein comprise fewer “ATG” sequence(s) across the length of a VP1 capsid coding sequence (e.g., in frame or out of frame) that encodes a protoparvovirus variant VP1 capsid polypeptide due to a conservative amino acid substitution of one or more purines surrounding an “ATG” (e.g., at position −3 or +4 relative to the first position of a VP1 capsid coding sequence, e.g., a conservative amino acid substitution within a Kozak consensus sequence) relative to a protoparvovirus reference VP1 capsid coding sequence described herein. In some embodiments, constructs described herein comprise an alternative translation initiation sequence (e.g., CTG, TTG, ACG, ATC) to improve potency relative to constructs comprising an ATG initiation sequence.

In some embodiments, a protoparvovirus variant VP1 capsid polynucleotide comprises a VP1 capsid coding sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a sequence selected from SEQ ID NOs: 97-102.

In some embodiments, a protoparvovirus variant VP1 capsid polypeptide comprises a polypeptide sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a sequence selected from SEQ ID NOs: 103-110.

### Exemplary Variant VP1 Capsid Polypeptide Coding Sequences

Exemplary canine parvovirus (CPV) variant VP1 capsid polypeptide construct sequences may be or comprise a VP1 capsid coding sequence according to SEQ ID NO: 97.

CTGGCACCTCCGGCAAAGAGAGCCAGGAGAGGATATAAATATCTTGGGCC
TGGGAACAGTCTTGACCAAGGAGAACCAACTAACCCTTCTGACGCCGCTG
CAAAAGAACACGACGAAGCTTACGCTGCTTATCTTCGCTCTGGTAAAAAC
CCATACTTATATTTCTCGCCAGCAGATCAACGCTTTATAGATCAAACTAA
GGACGCTAAAGATTGGGGGGGGAAAATAGGACATTATTTTTTTAGAGCTA
AAAAGGCAATTGCTCCAGTATTAACTGATACACCAGATCATCCATCAACA
TCAAGACCAACAAAACCAACTAAAAGAAGTAAACCACCACCTCATATTTT
CATCAATCTTGCAAAAAAAAAAAAAGCCGGTGCAGGACAAGTAAAAAGAG
ACAATCTTGCACCAATGAGTGATGGAGCAGTTCAACCAGACGGTGGTCAA
CCTGCTGTCAGAAATGAAAGAGCTACAGGATCTGGGAACGGGTCTGGAGG
CGGGGGTGGTGGTGGTTCTGGGGGTGTGGGGATTTCTACGGGTACTTTCA
ATAATCAGACGGAATTTAAATTTTTGGAAAACGGATGGGTGGAAATCACA
GCAAACTCAAGCAGACTTGTACATTTAAATATGCCAGAAAGTGAAAATTA
TAGAAGAGTGGTTGTAAATAATATGGATAAAACTGCAGTTAACGGAAACA
TGGCTTTAGATGATATTCATGCACAAATTGTAACACCTTGGTCATTGGTT
GATGCAAATGCTTGGGGAGTTTGGTTTAATCCAGGAGATTGGCAACTAAT
TGTTAATACTATGAGTGAGTTGCATTTAGTTAGTTTTGAACAAGAAATTT
TTAATGTTGTTTTAAAGACTGTTTCAGAATCTGCTACTCAGCCACCAACT
AAAGTTTATAATAATGATTTAACTGCATCATTGATGGTTGCATTAGATAG
TAATAATACTATGCCATTTACTCCAGCAGCTATGAGATCTGAGACATTGG
GTTTTTATCCATGGAAACCAACCATACCAACTCCATGGAGATATTATTTT
CAATGGGATAGAACATTAATACCATCTCATACTGGAACTAGTGGCACACC
AACAAATATATACCATGGTACAGATCCAGATGATGTTCAATTTTATACTA
TTGAAAATTCTGTGCCAGTACACTTACTAAGAACAGGTGATGAATTTGCT
ACAGGAACATTTTTTTTTGATTGTAAACCATGTAGACTAACACATACATG
GCAAACAAATAGAGCATTGGGCTTACCACCATTTCTAAATTCTTTGCCTC
AATCTGAAGGAGCTACTAACTTTGGTGATATAGGAGTTCAACAAGATAAA
AGACGTGGTGTAACTCAAATGGGAAATACAAACTATATTACTGAAGCTAC
TATTATGAGACCAGCTGAGGTTGGTTATAGTGCACCATATTATTCTTTTG
AGGCGTCTACACAAGGGCCATTTAAAACACCTATTGCAGCAGGACGGGGG
GGAGCGCAAACATATGAAAATCAAGCAGCAGATGGTGATCCAAGATATGC
ATTTGGTAGACAACATGGTCAAAAAACTACCACAACAGGAGAAACACCTG
AGAGATTTACATATATAGCACATCAAGATACAGGAAGATATCCAGAAGGA
GATTGGATTCAAAATATTAACTTTAACCTTCCTGTAACGAATGATAATGT
ATTGCTACCAACAGATCCAATTGGAGGTAAAACAGGAATTAACTATACTA
ATATATTTAATACTTATGGTCCTTTAACTGCATTAAATAATGTACCACCA
GTTTATCCAAATGGTCAAATTTGGGATAAAGAATTTGATACTGACTTAAA
ACCAAGACTTCATGTAAATGCACCATTTGTTTGTCAAAATAATTGTCCTG
GTCAATTATTTGTAAAAGTTGCGCCTAATTTAACAAATGAATATGATCCT
GATGCATCTGCTAATATGTCAAGAATTGTAACTTACTCAGATTTTTGGTG
GAAAGGTAAATTAGTATTTAAAGCTAAACTAAGAGCCTCTCATACTTGGA
ATCCAATTCAACAAATGAGTATTAATGTAGATAACCAATTTAACTATGTA
CCAAGTAATATTGGAGGTATGAAAATTGTATATGAAAAATCTCAACTAGC
ACCTAGAAAATTATATTAA

Exemplary cutavirus variant VP1 capsid polypeptide construct sequences may be or comprise a VP1 capsid coding sequence according to SEQ ID NO: 98.

CTGGCTCCAGCTATTAGAAAAGCCAGAGGTTACAACTTCCTAGGACCCTT
CAATCAAGACTTCAACAAAGAACCAACTAATCCATCAGACAACGCTGCAA
AACAACACGATTTGGAATACAACAAACTAATCAACCAAGGACACAATCCT
TATTGGTACTACAACAAAGCTGACGAAGACTTCATCAAAGCAACAGATCA
AGCACCAGACTGGGGAGGAAAATTTGGCAACTTCATCTTCAGAGCCAAAA
AACACATCGCTCCAGAACTGGCACCACCAGCAAAAAAGAAAAGCAAAACC
AAACACAGTGAACCAGAATTCAGCCACAAACACATCAAACCAGGCACCAA
AAGAGGTAAGCCTTTTCATATTTTTGTAAACCTTGCTAGAAAAAGAGCCC
GC

Exemplary cutavirus variant VP1 capsid polypeptide construct sequences may be or comprise a VP1 capsid coding sequence according to SEQ ID NO: 99.

ACGCCAGCTATTAGAAAAGCCAGAGGACCCTTCAATCAAGACTTCAACAA
AGAACCAACTAATCCATCAGACAACGCTGCAAAACAACACGATTTGGAAT
ACAACAAACTAATCAACCAAGGACACAATCCTTATTGGTACTACAACAAA
GCTGACGAAGACTTCATCAAAGCAACAGATCAAGCACCAGACTGGGGAGG
AAAATTTGGCAACTTCATCTTCAGAGCCAAAAAACACATCGCTCCAGAAC
TGGCACCACCAGCAAAAAAGAAAAGCAAAACCAAACACAGTGAACCAGAA
TTCAGCCACAAACACATCAAACCAGGCACCAAAAGAGGTAAGCCTTTTCA
TATTTTTGTAAACCTTGCTAGAAAAAGAGCCCGCATGTCAGAACCAGCTA
ATGATACAAATGAACAACCAGACAACTCCCCTGTTGAACAGGGTGCTGGT
CAAATTGGAGGAGGTGGAGGTGGAGGTGGAAGCGGTGTCGGGCACAGCAC
TGGTGATTATAATAATAGGACTGAGTTTATTTATCATGGTGATGAAGTCA
CAATTATTTGCCACTCTACAAGACTGGTTCACATCAATATGTCAGACAGG
GAAGACTACATCATCTATGAAACAGACAGAGGACCACTCTTTCCTACCAC
TCAGGACCTGCAGGGTAGAGACACTCTAAATGACTCTTACCATGCCAAAG
TAGAAACACCATGGAAACTACTCCATGCAAACAGCTGGGGCTGCTGGTTT
TCACCAGCAGACTTCCAACAAATGATCACCACATGCAGAGACATAGCACC
AATAAAAATGCACCAAAAAATAGAAAACATTGTCATCAAAACAGTCAGTA
AAACAGGCACAGGAGAAACAGAAACAACCAACTACAACAATGACCTCACA
GCACTCCTACAAATTGCACAAGACAACAGTAACCTACTACCATGGGCTGC
AGATAACTTTTATATAGACTCAGTAGGTTACGTTCCATGGAGAGCATGCA
AACTACCAACCTACTGCTACCACGTAGACACTTGGAATACAATTGACATA
AACCAAGCAGACACACCAAACCAATGGAGAGAAATCAAAAAAGGCATCCA
ATGGGACAATATCCAATTCACACCACTAGAAACTATGATAAACATTGACT
TACTAAGAACAGGAGATGCCTGGGAATCTGGTAACTACAATTTCCACACA
AAACCAACAAACCTAGCTTACCATTGGCAATCACAAAGACACACAGGCAG
CTGTCACCCAACAGTAGCACCTCTAGTTGAAAGAGGACAAGGAACCAACA
TACAATCAGTAAACTGTTGGCAATGGGGAGACAGAAACAATCCAAGCTCT
GCATCAACCAGAGTATCCAATATACATATTGGATACTCATTTCCAGAATG
GCAAATCCACTACTCAACAGGAGGACCAGTAATTAATCCAGGCAGTGCAT
TCTCACAAGCACCATGGGGCTCAACAACTGAAGGCACCAGACTAACCCAA
GGTGCATCTGAAAAAGCCATCTATGACTGGTCCCATGGAGATGACCAACC
AGGAGCCAGAGAAACCTGGTGGCAAAACAACCAACATGTAACAGGACAAA
CTGACTGGGCACCAAAAAATGCACACACCTCAGAACTCAACAACAATGTA
CCAGCAGCCACACACTTCTGGAAAAACAGCTATCACAACACCTTCTCACC
ATTCACTGCAGTAGATGATCATGGACCACAATATCCATGGGGAGCCATCT
GGGGAAAATACCCAGACACCACACACAAACCAATGATGTCAGCTCACGCA
CCATTCCTACTTCATGGACCACCTGGACAACTCTTTGTAAAACTAGCACC
AAACTATACAGACACACTTGACAACGGAGGTGTAACACATCCCAGAATCG
TCACATATGGAACCTTCTGGTGGTCAGGACAACTCATCTTTAAAGGAAAA
CTACGCACTCCAAGACAATGGAATACCTACAACCTACCAAGCCTAGACAA
AAGAGAAACCATGAAAAACACAGTACCAAATGAAGTTGGTCACTTTGAAC
TACCATACATGCCAGGAAGATGTCTACCAAACTACACATTGTAA

Exemplary feline panleukopenia virus variant VP1 capsid polypeptide construct sequences may be or comprise a VP1 capsid coding sequence according to SEQ ID NO: 100.

CTGGCACCTCCGGCAAAGAGAGCCAGGAGAGGATATAAATATCTTGGGCC
TGGGAACAGTCTTGACCAAGGAGAACCAACTAACCCTTCTGACGCCGCTG
CAAAAGAACACGACGAAGCTTACGCTGCTTATCTTCGCTCTGGTAAAAAC
CCATACTTATATTTCTCGCCAGCAGATCAACGCTTTATAGATCAAACTAA
GGACGCTAAAGATTGGGGGGGGAAAATAGGACATTATTTTTTTAGAGCTA
AAAAGGCAATTGCTCCAGTATTAACTGATACACCAGATCATCCATCAACA
TCAAGACCAACAAAACCAACTAAAAGAAGTAAACCACCACCTCATATTTT
CATCAATCTTGCAAAAAAAAAAAAAGCCGGTGCAGGACAAGTAAAAAGAG
ACAATCTTGCACCAATGAGTGATGGAGCAGTTCAACCAGACGGTGGTCAA
CCTGCTGTCAGAAATGAAAGAGCTACAGGATCTGGGAACGGGTCTGGAGG
CGGGGGTGGTGGTGGTTCTGGGGGTGTGGGGATTTCTACGGGTACTTTCA
ATAATCAGACGGAATTTAAATTTTTGGAAAACGGATGGGTGGAAATCACA
GCAAACTCAAGCAGACTTGTACATTTAAATATGCCAGAAAGTGAAAATTA
TAAAAGAGTAGTTGTAAATAATATGGATAAAACTGCAGTTAAAGGAAACA
TGGCTTTAGATGATATTCATGTACAAATTGTAACACCTTGGTCATTGGTT
GATGCAAATGCTTGGGGAGTTTGGTTTAATCCAGGAGATTGGCAACTAAT
TGTTAATACTATGAGTGAGTTGCATTTAGTTAGTTTTGAACAAGAAATTT
TTAATGTTGTTTTAAAGACTGTTTCAGAATCTGCTACTCAGCCACCAACT
AAAGTTTATAATAATGATTTAACTGCATCATTGATGGTTGCATTAGATAG
TAATAATACTATGCCATTTACTCCAGCAGCTATGAGATCTGAGACATTGG
GTTTTTATCCATGGAAACCAACCATACCAACTCCATGGAGATATTATTTT
CAATGGGATAGAACATTAATACCATCTCATACTGGAACTAGTGGCACACC
AACAAATATATACCATGGTACAGATCCAGATGATGTTCAATTTTATACTA
TTGAAAATTCTGTGCCAGTACACTTACTAAGAACAGGTGATGAATTTGCT
ACAGGAACATTTTTTTTTGATTGTAAACCATGTAGACTAACACATACATG
GCAAACAAATAGAGCATTGGGCTTACCACCATTTTTAAATTCTTTGCCTC
AATCTGAAGGAGCTACTAACTTTGGTGATATAGGAGTTCAACAAGATAAA
AGACGTGGTGTAACTCAAATGGGAAATACAAACTATATTACTGAAGCTAC
TATTATGAGACCAGCTGAGGTTGGTTATAGTGCACCATATTATTCTTTTG
AGGCGTCTACACAAGGGCCATTTAAAACACCTATTGCAGCAGGACGGGGG
GGAGCGCAAACAGATGAAAATCAAGCAGCAGATGGTGATCCAAGATATGC
ATTTGGTAGACAACATGGTCAAAAAACTACCACAACAGGAGAAACACCTG
AGAGATTTACATATATAGCACATCAAGATACAGGAAGATATCCAGAAGGA
GATTGGATTCAAAATATTAACTTTAACCTTCCTGTAACAAATGATAATGT
ATTGCTACCAACAGATCCAATTGGAGGTAAAACAGGAATTAACTATACTA
ATATATTTAATACTTATGGTCCTTTAACTGCATTAAATAATGTACCACCA
GTTTATCCAAATGGTCAAATTTGGGATAAAGAATTTGATACTGACTTAAA
ACCAAGACTTCATGTAAATGCACCATTTGTTTGTCAAAATAATTGTCCTG
GTCAATTATTTGTAAAAGTTGCGCCTAATTTAACAAATGAATATGATCCT
GATGCATCTGCTAATATGTCAAGAATTGTAACTTACTCAGATTTTTGGTG
GAAAGGTAAATTAGTATTTAAAGCTAAACTAAGAGCCTCTCATACTTGGA
ATCCAATTCAACAAATGAGTATTAATGTAGATAACCAATTTAACTATGTA
CCAAGTAATATTGGAGCTATGAAAATTGTATATGAAAAATCTCAACTAGC
ACCTAGAAAATTATATTAA

Exemplary minute virus of mice variant VP1 capsid polypeptide construct sequences may be or comprise a VP1 capsid coding sequence according to SEQ ID NO: 101.

ACGGCGCCTCCAGCTAAAAGAGCTAAAAGAGGCTACAAGTACCTGGGACC
AGGGAACAGCCTTGACCAAGGAGAACCAACCAATCCATCTGACGCCGCTG
CCAAAGAGCACGACGAGGCCTACGATCAATACATCAAATCTGGAAAAAAT
CCTTACCTGTACTTCTCTGCTGCTGATCAACGCTTTATTGACCAAACCAA
GGACGCCAAAGACTGGGGAGGCAAGGTTGGTCACTACTTTTTTAGAACCA
AGCGCGCTTTTGCACCTAAGCTTGCTACTGACTCTGAACCTGGAACTTCT
GGTGTAAGCAGAGCTGGTAAACGCACTAGACCACCTGCTTACATTTTTAT
TAACCAAGCCAGAGCTAAAAAAAAACTTACTTCTTCTGCTGCACAGCAAA
GCAGTCAAACCATGAGTGATGGCACCAGCCAACCTGACAGCGGAAACGCT
GTCCACTCAGCTGCAAGAGTTGAACGAGCAGCTGACGGCCCTGGAGGCTC
TGGGGGTGGGGGCTCTGGCGGGGGTGGGGTTGGTGTTTCTACTGGGTCTT
ATGATAATCAAACGCATTATAGATTCTTGGGTGACGGCTGGGTAGAAATT
ACTGCACTAGCAACTAGACTAGTACATTTAAACATGCCTAAATCAGAAAA
CTATTGCAGAATCAGAGTTCACAATACAACAGACACATCAGTCAAAGGCA
ACATGGCAAAAGATGATGCTCATGAGCAAATTTGGACACCATGGAGCTTG
GTGGATGCTAATGCTTGGGGAGTTTGGCTCCAGCCAAGTGACTGGCAATA
CATTTGCAACACCATGAGCCAGCTTAACTTGGTATCACTTGATCAAGAAA
TATTCAATGTAGTGCTGAAAACTGTTACAGAGCAAGACTTAGGAGGTCAA
GCTATAAAAATATACAACAATGACCTTACAGCTTGCATGATGGTTGCAGT
AGACTCAAACAACATTTTGCCATACACACCTGCAGCAAACTCAATGGAAA
CACTTGGTTTCTACCCCTGGAAACCAACCATAGCATCACCATACAGGTAC
TATTTTTGCGTTGACAGAGATCTTTCAGTGACCTACGAAAATCAAGAAGG
CACAGTTGAACATAATGTGATGGGAACACCAAAAGGAATGAATTCTCAAT
TTTTTACCATTGAGAACACACAACAAATCACATTGCTCAGAACAGGGGAC
GAATTTGCCACAGGTACTTACTACTTTGACACAAATTCAGTTAAACTCAC
ACACACGTGGCAAACCAACCGTCAACTTGGACAGCCTCCACTGCTGTCAA
CCTTTCCTGAAGCTGACACTGATGCAGGTACACTTACTGCTCAAGGGAGC
AGACATGGAACAACACAAATGGGGGTTAACTGGGTGAGTGAAGCAATCAG
AACCAGACCTGCTCAAGTAGGATTTTGTCAACCACACAATGACTTTGAAG
CCAGCAGAGCTGGACCATTTGCTGCCCCAAAAGTTCCAGCAGATATTACT
CAAGGAGTAGACAAAGAAGCCAATGGCAGTGTTAGATACAGTTATGGCAA
ACAGCATGGTGAAAATTGGGCTTCACATGGACCAGCACCAGAGCGCTACA
CATGGGATGAAACAAGCTTTGGTTCAGGTAGAGACACCAAAGATGGTTTT
ATTCAATCAGCACCACTAGTTGTTCCACCACCACTAAATGGCATTCTTAC
AAATGCAAACCCTATTGGGACTAAAAATGACATTCATTTTTCAAATGTTT
TTAACAGCTATGGTCCACTAACTGCATTTTCACACCCAAGTCCTGTATAC
CCTCAAGGACAAATATGGGACAAAGAACTAGATCTTGAACACAAACCTAG
ACTTCACATAACTGCTCCATTTGTTTGTAAAAACAATGCACCTGGACAAA
TGTTGGTTAGATTAGGACCAAACCTAACTGACCAATATGATCCAAACGGA
GCCACACTTTCTAGAATTGTTACATACGGTACATTTTTCTGGAAAGGAAA
ACTAACCATGAGAGCAAAACTTAGAGCTAACACCACTTGGAACCCAGTGT
ACCAAGTAAGTGCTGAAGACAATGGCAACTCATACATGAGTGTAACTAAA
TGGTTACCAACTGCTACTGGAAACATGCAGTCTGTGCCGCTTATAACAAG
ACCTGTTGCTAGAAATACTTACTAA

Exemplary rat H-1 parvovirus variant VP1 capsid polypeptide construct sequences may be or comprise a VP1 capsid coding sequence according to SEQ ID NO: 102.

ACGGCACCTCCAGCTAAAAGAGCTAAAAGAGGCTACAAGTACCTGGGACC
AGGGAACAGCCTTGACCAAGGAGAACCAACCAACCCTTCTGACGCCGCTG
CCAAAGAACACGACGAAGCCTACGACCAATACATCAAATCTGGAAAAAAT
CCTTACCTGTACTTCTCTCCTGCTGATCAACGCTTCATTGACCAAACCAA
AGACGCCAAGGACTGGGGCGGCAAGGTTGGTCACTACTTTTTTAGAACCA
AGCGAGCTTTTGCACCTAAGCTTTCTACTGACTCTGAACCTGGCACTTCT
GGTGTGAGCAGACCTGGTAAACGAACTAAACCACCTGCTCACATTTTTGT
AAATCAAGCCAGAGCTAAAAAAAAACGCGCTTCTCTTGCTGCACAGCAGA
GGACTCTGACAATGAGTGATGGCACCGAAACAAACCAACCAGACACTGGA
ATCGCTAATGCTAGAGTTGAGCGATCAGCTGACGGAGGTGGAAGCTCTGG
GGGTGGGGGCTCTGGCGGGGGTGGGATTGGTGTTTCTACTGGGACTTATG
ATAATCAAACGACTTATAAGTTTTTGGGAGATGGATGGGTAGAAATAACT
GCACATGCTTCTAGACTTTTGCACTTGGGAATGCCTCCTTCAGAAAACTA
CTGCCGCGTCACCGTTCACAATAATCAAACAACAGGACACGGAACTAAGG
TAAAGGGAAACATGGCCTATGATGACACACATCAACAAATTTGGACACCA
TGGAGCTTGGTAGATGCTAATGCTTGGGGAGTTTGGTTCCAACCAAGTGA
CTGGCAGTTCATTCAAAACAGCATGGAATCGCTGAATCTTGACTCATTGA
GCCAAGAACTATTTAATGTAGTAGTCAAAACAGTCACTGAACAACAAGGA
GCTGGCCAAGATGCCATTAAAGTCTATAATAATGACTTGACGGCCTGTAT
GATGGTTGCTCTGGATAGTAACAACATACTGCCTTACACACCTGCAGCTC
AAACATCAGAAACACTTGGTTTCTACCCATGGAAACCAACCGCACCAGCT
CCTTACAGATACTACTTTTTCATGCCTAGACAACTCAGTGTAACCTCTAG
CAACTCTGCTGAAGGAACTCAAATCACAGACACCATTGGAGAGCCACAGG
CACTAAACTCTCAATTTTTTACTATTGAGAACACCTTGCCTATTACTCTC
CTGCGCACAGGTGATGAGTTTACAACTGGCACCTACATCTTTAACACTGA
CCCACTTAAACTTACTCACACATGGCAAACCAACAGACACTTGGGCATGC
CTCCAAGAATAACTGACCTACCAACATCAGATACAGCAACAGCATCACTA
ACTGCAAATGGAGACAGATTTGGATCAACACAAACACAGAATGTGAACTA
TGTCACAGAGGCTTTGCGCACCAGGCCTGCTCAGATTGGCTTCATGCAAC
CTCATGACAACTTTGAAGCAAACAGAGGTGGCCCATTTAAGGTTCCAGTG
GTACCGCTAGACATAACAGCTGGCGAGGACCATGATGCAAACGGAGCCAT
ACGATTTAACTATGGCAAACAACATGGCGAAGATTGGGCCAAACAAGGAG
CAGCACCAGAAAGGTACACATGGGATGCAATTGATAGTGCAGCTGGGAGG
GACACAGCTAGATGCTTTGTACAAAGTGCACCAATATCTATTCCACCAAA
CCAAAACCAGATCTTGCAGCGAGAAGACGCCATAGCTGGCAGAACTAACA
TGCATTATACTAATGTTTTTAACAGCTATGGTCCACTTAGTGCATTTCCT
CATCCAGATCCCATTTATCCAAATGGACAAATTTGGGACAAAGAATTGGA
CCTGGAACACAAACCTAGACTACACGTAACTGCACCATTTGTTTGTAAAA
ACAACCCACCAGGTCAACTATTTGTTCGCTTGGGGCCTAATCTGACTGAC
CAATTTGACCCAAACAGCACAACTGTTTCTCGCATTGTTACATATAGCAC
TTTTTACTGGAAGGGTATTTTGAAATTCAAAGCCAAACTAAGACCAAATC
TGACCTGGAATCCTGTATACCAAGCAACCACAGACTCTGTTGCCAATTCT
TACATGAATGTTAAGAAATGGCTCCCATCTGCAACTGGCAACATGCACTC
TGATCCATTGATTTGTAGACCTGTGCCTCACATGACATACTAA

### Exemplary Variant VP1 Capsid Polypeptide Sequences

Exemplary bufavirus variant VP1 capsid polypeptide construct sequences may be or comprise a polypeptide sequence according to SEQ ID NO: 103.

MPAIRKARGYNYLGPFNQDFSKKPTNPSDNAARKHDLEYNKLIKQGHNPY
WNYNHADEDFIKETDQATDWGGKFGNFVFRAKRALAPELAPPAKKKTKTK
HTEPEYSHKHIKAGTKRGKPFYLFVNLARKKARMTDTQDVSEQQSDQPSV
ASTSAKAGGGGGGGGSGVGHSTGNYNNRTEFYYHGDEVTIVCHSSRHIHL
NMSESEEYKIYDTDRGPTFPTDQTLQGRDTINDSYHAQVETPWFLINPNS
WGTWMNPADFQQLTTTCREVTLEHLDQTLDNIVIKTVSKQGSGAEETTQY
NNDLTALLQVALDKSNQLPWVADNMYLDSLGYIPWRPCKLKQYSYHVNFW
NTIDIISGPQQNQWQQVKKEIKWDDLQFTPIETTTEIDLLRTGDSWTSGP
YKFNTKPTQLSYHWQSTRHTGSVHPTEPPNAIGQQGRNIIDINGWQWGDR
SNPMSAATRVSNFHIGYSWPEWRIHYGSGGPAINPGAPFSQAPWSTDPQV
RLTQGASEKAIFDYNHGDDDPAHRDQWWQNNLPMTGQTDWAPKNAHQTNV
SNNIPSRQEFWTQDYHNTFGPFTAVDDVGIQYPWGAIWTKTPDTTHKPMM
SAHAPFICKDGPPGQLLVKLAPNYTENLQTDGLGNNRIVTYATFWWTGKL
VLKGKLRLPRQFNLYNLPGRPRGTEAKKFLPNEIGHFELPFMPGRCMPNY
TI

Exemplary canine parvovirus (CPV) variant VP1 capsid polypeptide construct sequences may be or comprise a polypeptide sequence according to SEQ ID NO: 104.

LAPPAKRARRGYKYLGPGNSLDQGEPTNPSDAAAKEHDEAYAAYLRSGKN
PYLYFSPADQRFIDQTKDAKDWGGKIGHYFFRAKKAIAPVLTDTPDHPST
SRPTKPTKRSKPPPHIFINLAKKKKAGAGQVKRDNLAPMSDGAVQPDGGQ
PAVRNERATGSGNGSGGGGGGGSGGVGISTGTFNNQTEFKFLENGWVEIT
ANSSRLVHLNMPESENYRRVVVNNMDKTAVNGNMALDDIHAQIVTPWSLV
DANAWGVWFNPGDWQLIVNTMSELHLVSFEQEIFNVVLKTVSESATQPPT
KVYNNDLTASLMVALDSNNTMPFTPAAMRSETLGFYPWKPTIPTPWRYYF
QWDRTLIPSHTGTSGTPTNIYHGTDPDDVQFYTIENSVPVHLLRTGDEFA
TGTFFFDCKPCRLTHTWQTNRALGLPPFLNSLPQSEGATNFGDIGVQQDK
RRGVTQMGNTNYITEATIMRPAEVGYSAPYYSFEASTQGPFKTPIAAGRG
GAQTYENQAADGDPRYAFGRQHGQKTTTTGETPERFTYIAHQDTGRYPEG
DWIQNINFNLPVTNDNVLLPTDPIGGKTGINYTNIFNTYGPLTALNNVPP
VYPNGQIWDKEFDTDLKPRLHVNAPFVCQNNCPGQLFVKVAPNLTNEYDP
DASANMSRIVTYSDFWWKGKLVFKAKLRASHTWNPIQQMSINVDNQFNYV
PSNIGGMKIVYEKSQLAPRKLY

Exemplary cutavirus variant VP1 capsid polypeptide construct sequences may be or comprise a polypeptide sequence according to SEQ ID NO: 105.

MPAIRKARGYNFLGPFNQDENKEPTNPSDNAAKQHDLEYNKLINQGHNPY
WYYNKADEDFIKATDQAPDWGGKFGNFIFRAKKHIAPELAPPAKKKSKTK
HPEPEFSHKHIKPGTKRGKPFHIFVNLARKRARMSEPAENTNDQPNDSPV
EQGAGQIGGGGGGGGSGVGHSTGDYNNRTEFIYHGDEVTIICHSTRLVHI
NMSDREDYIIYETDRGQLFPTTQDLQGRDTLNDSYHAKVETPWKLLHANS
WGCWFSPADFQQMITTCRDIAPIQMHQKIENIVIKTVSKTGTGETETTNY
NNDLTALLQIAQDNSNLLPWAADNFYIDSVGYVPWRACKLPTYCYHVDTW
NTIDINQADAPNRWREIKKGIQWDNIQFTPLETMINIDLLRTGDAWQSGN
YNFHTKPTNLAYHWQSQRHTGSCHPTVAPLVERGQGTNIQSVNCWQWGDR
NNPSSASTRVSNMHIGYSFPEWQIHYSTGGPVINPGSAFSQAPWGSTTEG
TRLTQGASEKAIYDWAHGDDQPGARETWWQNNQHVTGQTDWAPKNAHTSE
LNNNVPAATHFWKNSYHNTFSPFTAVDDHGPQYPWGAIWGKYPDTTHKPM
MSAHAPFLLHGPPGQLFVKLAPNYTDTLDNGGVTHPRIVTYGTFWWSGKL
IFKGKLRTPRQWNTYNLPSLDKRETMKNTVPNEVGHFELPYMPGRCLPNY
TL

Exemplary cutavirus variant VP1 capsid polypeptide construct sequences may be or comprise a polypeptide sequence according to SEQ ID NO: 106.

TPAIRKARGPFNQDFNKEPTNPSDNAAKQHDLEYNKLINQGHNPYWYYNK
ADEDFIKATDQAPDWGGKFGNFIFRAKKHIAPELAPPAKKKSKTKHSEPE
FSHKHIKPGTKRGKPFHIFVNLARKRARMSEPANDTNEQPDNSPVEQGAG
QIGGGGGGGGSGVGHSTGDYNNRTEFIYHGDEVTIICHSTRLVHINMSDR
EDYIIYETDRGPLFPTTQDLQGRDTLNDSYHAKVETPWKLLHANSWGCWF
SPADFQQMITTCRDIAPIKMHQKIENIVIKTVSKTGTGETETTNYNNDLT
ALLQIAQDNSNLLPWAADNFYIDSVGYVPWRACKLPTYCYHVDTWNTIDI
NQADTPNQWREIKKGIQWDNIQFTPLETMINIDLLRTGDAWESGNYNFHT
KPTNLAYHWQSQRHTGSCHPTVAPLVERGQGTNIQSVNCWQWGDRNNPSS
ASTRVSNIHIGYSFPEWQIHYSTGGPVINPGSAFSQAPWGSTTEGTRLTQ
GASEKAIYDWSHGDDQPGARETWWQNNQHVTGQTDWAPKNAHTSELNNNV
PAATHFWKNSYHNTFSPFTAVDDHGPQYPWGAIWGKYPDTTHKPMMSAHA
PFLLHGPPGQLFVKLAPNYTDTLDNGGVTHPRIVTYGTFWWSGQLIFKGK
LRTPRQWNTYNLPSLDKRETMKNTVPNEVGHFELPYMPGRCLPNYTL

Exemplary feline panleukopenia virus variant VP1 capsid polypeptide construct sequences may be or comprise a polypeptide sequence according to SEQ ID NO: 107.

LAPPAKRARRGYKYLGPGNSLDQGEPTNPSDAAAKEHDEAYAAYLRSGKN
PYLYFSPADQRFIDQTKDAKDWGGKIGHYFFRAKKAIAPVLTDTPDHPST
SRPTKPTKRSKPPPHIFINLAKKKKAGAGQVKRDNLAPMSDGAVQPDGGQ
PAVRNERATGSGNGSGGGGGGGSGGVGISTGTFNNQTEFKFLENGWVEIT
ANSSRLVHLNMPESENYKRVVVNNMDKTAVKGNMALDDIHVQIVTPWSLV
DANAWGVWFNPGDWQLIVNTMSELHLVSFEQEIFNVVLKTVSESATQPPT
KVYNNDLTASLMVALDSNNTMPFTPAAMRSETLGFYPWKPTIPTPWRYYF
QWDRTLIPSHTGTSGTPTNIYHGTDPDDVQFYTIENSVPVHLLRTGDEFA
TGTFFFDCKPCRLTHTWQTNRALGLPPFLNSLPQSEGATNFGDIGVQQDK
RRGVTQMGNTNYITEATIMRPAEVGYSAPYYSFEASTQGPFKTPIAAGRG
GAQTDENQAADGDPRYAFGRQHGQKTTTTGETPERFTYIAHQDTGRYPEG
DWIQNINFNLPVTNDNVLLPTDPIGGKTGINYTNIFNTYGPLTALNNVPP
VYPNGQIWDKEFDTDLKPRLHVNAPFVCQNNCPGQLFVKVAPNLTNEYDP
DASANMSRIVTYSDFWWKGKLVFKAKLRASHTWNPIQQMSINVDNQFNYV
PSNIGAMKIVYEKSQLAPRKLY

Exemplary minute virus of mice variant VP1 capsid polypeptide construct sequences may be or comprise a polypeptide sequence according to SEQ ID NO: 108.

TAPPAKRAKRGYKYLGPGNSLDQGEPTNPSDAAAKEHDEAYDQYIKSGKN
PYLYFSAADQRFIDQTKDAKDWGGKVGHYFFRTKRAFAPKLATDSEPGTS
GVSRAGKRTRPPAYIFINQARAKKKLTSSAAQQSSQTMSDGTSQPDSGNA
VHSAARVERAADGPGGSGGGGSGGGGVGVSTGSYDNQTHYRFLGDGWVEI
TALATRLVHLNMPKSENYCRIRVHNTTDTSVKGNMAKDDAHEQIWTPWSL
VDANAWGVWLQPSDWQYICNTMSQLNLVSLDQEIFNVVLKTVTEQDLGGQ
AIKIYNNDLTACMMVAVDSNNILPYTPAANSMETLGFYPWKPTIASPYRY
YFCVDRDLSVTYENQEGTVEHNVMGTPKGMNSQFFTIENTQQITLLRTGD
EFATGTYYFDTNSVKLTHTWQTNRQLGQPPLLSTFPEADTDAGTLTAQGS
RHGTTQMGVNWVSEAIRTRPAQVGFCQPHNDFEASRAGPFAAPKVPADIT
QGVDKEANGSVRYSYGKQHGENWASHGPAPERYTWDETSFGSGRDTKDGF
IQSAPLVVPPPLNGILTNANPIGTKNDIHFSNVFNSYGPLTAFSHPSPVY
PQGQIWDKELDLEHKPRLHITAPFVCKNNAPGQMLVRLGPNLTDQYDPNG
ATLSRIVTYGTFFWKGKLTMRAKLRANTTWNPVYQVSAEDNGNSYMSVTK
WLPTATGNMQSVPLITRPVARNTY

Exemplary tusavirus variant VP1 capsid polypeptide construct sequences may be or comprise a polypeptide sequence according to SEQ ID NO: 109.

MAPAARPRKGYNYLGPGNDLDAGEPTNKSDAAARKHDFAYSAYLKQGLDP
YWNFNKADEKFIRDTEGATDWGGRLGHWIFRAKKHILPHLKEPTLAGRKR
PAPAHIFVNLANKRKKGLPTRKDQQKDTLDSNAQQPVREADQPDGMAASS
SDSGPSSSGGGARAGGVGVSTGDFDNTTLWDFHEDGTATITCNSTRLVHL
TRPDSLDYKIIPTQNNTAVQTVGHMMDDDNHTQVLTPWSLVDCNAWGVWL
SPHDWQHIMNIGEELELLSLEQEVFNVTLKTATETGPPESRITMYNNDLT
AVMMITTDTNNQLPYTPAAIRSETLGFYPWRPTVVPRWRYYFDWDRFLSV
TSSSDQSTSIINHSSTQSAIGQFFVIETQLPIALLRTGDSYATGGYKFDC
NKVNLGRHWQTTRSLGLPPKIEPPTSESALGTINQNARLGWRWGINDVHE
TNVVRPCTAGYNHPEWFYTHTLEGPAIDPAPPTSIPSNWGGGTPPDTRAS
SHNQQRITYNYNHGNKDENLNNFSLNPNIELGSIINQGNFLSYEGNGQQI
NTTAGVGKNGETATSDPNLVRYMPNTYGVYTAVDHQGPVYPHGQIWDKQI
HTDKKPELHCLAPFTCKNNPPGQMFVRIAPNLTDTFNATPTFSEIITYAD
FWWKGTLKMKIKLRPPHQWNIATVLGAAVNIGDAARFVPNRLGQLEFPVI
NGRIVPSTVY

Exemplary rat H-1 parvovirus variant VP1 capsid polypeptide construct sequences may be or comprise a polypeptide sequence according to SEQ ID NO: 110.

TAPPAKRAKRGYKYLGPGNSLDQGEPTNPSDAAAKEHDEAYDQYIKSGKN
PYLYFSPADQRFIDQTKDAKDWGGKVGHYFFRTKRAFAPKLSTDSEPGTS
GVSRPGKRTKPPAHIFVNQARAKKKRASLAAQQRTLTMSDGTETNQPDTG
IANARVERSADGGGSSGGGGSGGGGIGVSTGTYDNQTTYKFLGDGWVEIT
AHASRLLHLGMPPSENYCRVTVHNNQTTGHGTKVKGNMAYDDTHQQIWTP
WSLVDANAWGVWFQPSDWQFIQNSMESLNLDSLSQELFNVVVKTVTEQQG
AGQDAIKVYNNDLTACMMVALDSNNILPYTPAAQTSETLGFYPWKPTAPA
PYRYYFFMPRQLSVTSSNSAEGTQITDTIGEPQALNSQFFTIENTLPITL
LRTGDEFTTGTYIFNTDPLKLTHTWQTNRHLGMPPRITDLPTSDTATASL
TANGDRFGSTQTQNVNYVTEALRTRPAQIGFMQPHDNFEANRGGPFKVPV
VPLDITAGEDHDANGAIRFNYGKQHGEDWAKQGAAPERYTWDAIDSAAGR
DTARCFVQSAPISIPPNQNQILQREDAIAGRTNMHYTNVFNSYGPLSAFP
HPDPIYPNGQIWDKELDLEHKPRLHVTAPFVCKNNPPGQLFVRLGPNLTD
QFDPNSTTVSRIVTYSTFYWKGILKFKAKLRPNLTWNPVYQATTDSVANS
YMNVKKWLPSATGNMHSDPLICRPVPHMTY

iii. Exemplary VP2 Capsid Sequences

In some embodiments, constructs, compositions, virions, or populations of virions comprise a coding sequence that encodes a protoparvovirus VP2 capsid polypeptide. In some embodiments, a protoparvovirus VP2 polypeptide of a protoparvovirus is encoded by a coding sequence with at least 85%, 90%, 95%, 98% or 99% sequence identity to a coding sequence described herein. In some embodiments, a protoparvovirus VP2 capsid polypeptide of a protoparvovirus comprises a polypeptide with at least 85%, 90%, 95%, 98% or 99% sequence identity to a polypeptide of a sequence described herein.

### Exemplary Bufavirus (BuV) VP2 Sequences

Exemplary bufavirus VP2 capsid polypeptide sequences may be or comprise a polypeptide sequence according to SEQ ID NO: 111.

MTDTQDVSEQQSDQPSVASTSAKAGGGGGGGGSGVGHSTGNYNNRTEFYY
HGDEVTIVCHSSRHIHLNMSESEEYKIYDTDRGPTFPTDQTLQGRDTIND
SYHAQVETPWFLINPNSWGTWMNPADFQQLTTTCREVTLEHLDQTLDNIV
IKTVSKQGSGAEETTQYNNDLTALLQVALDKSNQLPWVADNMYLDSLGYI
PWRPCKLKQYSYHVNFWNTIDIISGPQQNQWQQVKKEIKWDDLQFTPIET
TTEIDLLRTGDSWTSGPYKFNTKPTQLSYHWQSTRHTGSVHPTEPPNAIG
QQGRNIIDINGWQWGDRSNPMSAATRVSNFHIGYSWPEWRIHYGSGGPAI
NPGAPFSQAPWSTDPQVRLTQGASEKAIFDYNHGDDDPAHRDQWWQNNLP
MTGQTDWAPKNAHQTNVSNNIPSRQEFWTQDYHNTFGPFTAVDDVGIQYP
WGAIWTKTPDTTHKPMMSAHAPFICKDGPPGQLLVKLAPNYTENLQTDGL
GNNRIVTYATFWWTGKLVLKGKLRLPRQFNLYNLPGRPRGTEAKKFLPNE
IGHFELPFMPGRCMPNYTI

### Exemplary Canine Parvovirus (CPV) VP2 Sequences

Exemplary canine parvovirus (CPV) VP2 capsid polypeptide sequences may be or comprise a polypeptide sequence according to SEQ ID NO: 112.

MSDGAVQPDGGQPAVRNERATGSGNGSGGGGGGGSGGVGISTGTFNNQTE
FKFLENGWVEITANSSRLVHLNMPESENYRRVVVNNLDKTAVNGNMALDD
THAQIVTPWSLVDANAWGVWFNPGDWQLIVNTMSELHLVSFEQEIFNVVL
KTVSESATQPPTKVYNNDLTASLMVALDSNNTMPFTPAAMRSETLGFYPW
KPTIPTPWRYYFQWDRTLIPSHTGTSGTPTNIYHGTDPDDVQFYTIENSV
PVHLLRTGDEFATGTFFFDCKPCRLTHTWQTNRALGLPPFLNSLPQSEGG
TNFGYIGVQQDKRRGVTQMGNTNYITEATIMRPAEVGYSAPYYSFEASTQ
GPFKTPIAAGRGGAQTDENQAADGDPRYAFGRQHGQKTTTTGETPERFTY
IAHQDTGRYPEGDWIQNINFNLPVTDDNVLLPTDPIGGKTGINYTNIFNT
YGPLTALNNVPPVYPNGQIWDKEFDTDLKPRLHVNAPFVCQNNCPGQLFV
KVAPNLTNEYDPDASANMSRIVTYSDFWWKGKLVFKAKLRASHTWNPIQQ
MSINVDNQFNYVPSNIGGMKIVYEKSQLAPRKLY

Exemplary canine parvovirus (CPV) VP2 capsid polypeptide sequences may be or comprise a coding sequence according to SEQ ID NO: 113.

ATGAGCGACGGCGCCGTGCAGCCCGACGGCGGCCAGCCCGCCGTGCGCAA
CGAGCGCGCCACCGGCAGCGGCAACGGCAGCGGCGGCGGCGGCGGCGGCG
GCAGCGGCGGCGTGGGCATCAGCACCGGCACCTTCAACAACCAGACCGAG
TTCAAGTTCCTGGAGAACGGCTGGGTGGAGATCACCGCCAACAGCAGCCG
CCTGGTGCACCTGAACATGCCCGAGAGCGAGAACTACCGCCGCGTGGTGG
TGAACAACATGGACAAGACCGCCGTGAACGGCAACATGGCCCTGGACGAC
ATCCACGCCCAGATCGTGACCCCCTGGAGCCTGGTGGACGCCAACGCCTG
GGGCGTGTGGTTCAACCCCGGCGACTGGCAGCTGATCGTGAACACCATGA
GCGAGCTGCACCTGGTGAGCTTCGAGCAGGAGATCTTCAACGTGGTGCTG
AAGACCGTGAGCGAGAGCGCCACCCAGCCCCCCACCAAGGTGTACAACAA
CGACCTGACCGCCAGCCTGATGGTGGCCCTGGACAGCAACAACACCATGC
CCTTCACCCCCGCCGCCATGCGCAGCGAGACCCTGGGCTTCTACCCCTGG
AAGCCCACCATCCCCACCCCCTGGCGCTACTACTTCCAGTGGGACCGCAC
CCTGATCCCCAGCCACACCGGCACCAGCGGCACCCCCACCAACATCTACC
ACGGCACCGACCCCGACGACGTGCAGTTCTACACCATCGAGAACAGCGTG
CCCGTGCACCTGCTGCGCACCGGCGACGAGTTCGCCACCGGCACCTTCTT
CTTCGACTGCAAGCCCTGCCGCCTGACCCACACCTGGCAGACCAACCGCG
CCCTGGGCCTGCCCCCCTTCCTGAACAGCCTGCCCCAGAGCGAGGGCGCC
ACCAACTTCGGCGACATCGGCGTGCAGCAGGACAAGCGCCGCGGCGTGAC
CCAGATGGGCAACACCAACTACATCACCGAGGCCACCATCATGCGCCCCG
CCGAGGTGGGCTACAGCGCCCCCTACTACAGCTTCGAGGCCAGCACCCAG
GGCCCCTTCAAGACCCCCATCGCCGCCGGCCGCGGCGGCGCCCAGACCTA
CGAGAACCAGGCCGCCGACGGCGACCCCCGCTACGCCTTCGGCCGCCAGC
ACGGCCAGAAGACCACCACCACCGGCGAGACCCCCGAGCGCTTCACCTAC
ATCGCCCACCAGGACACCGGCCGCTACCCCGAGGGCGACTGGATCCAGAA
CATCAACTTCAACCTGCCCGTGACCAACGACAACGTGCTGCTGCCCACCG
ACCCCATCGGCGGCAAGACCGGCATCAACTACACCAACATCTTCAACACC
TACGGCCCCCTGACCGCCCTGAACAACGTGCCCCCCGTGTACCCCAACGG
CCAGATCTGGGACAAGGAGTTCGACACCGACCTGAAGCCCCGCCTGCACG
TGAACGCCCCCTTCGTGTGCCAGAACAACTGCCCCGGCCAGCTGTTCGTG
AAGGTGGCCCCCAACCTGACCAACGAGTACGACCCCGACGCCAGCGCCAA
CATGAGCCGCATCGTGACCTACAGCGACTTCTGGTGGAAGGGCAAGCTGG
TGTTCAAGGCCAAGCTGCGCGCCAGCCACACCTGGAACCCCATCCAGCAG
ATGAGCATCAACGTGGACAACCAGTTCAACTACGTGCCCAGCAACATCGG
CGGCATGAAGATCGTGTACGAGAAGAGCCAGCTGGCCCCCCGCAAGCTGT
AC

### Exemplary Cutavirus (CuV) Parvovirus VP2 Sequences

Exemplary cutavirus VP2 capsid polypeptide sequences may be or comprise a polypeptide sequence according to SEQ ID NO: 114.

MSEPANDTNEQPDNSPVEQGAGQIGGGGGGGGSGVGHSTGDYNNRTEFIY
HGDEVTIICHSTRLVHINMSDREDYIIYETDRGPLFPTTQDLQGRDTLND
SYHAKVETPWKLLHANSWGCWFSPADFQQMITTCRDIAPIKMHQKIENIV
IKTVSKTGTGETETTNYNNDLTALLQIAQDNSNLLPWAADNFYIDSVGYV
PWRACKLPTYCYHVDTWNTIDINQADTPNQWREIKKGIQWDNIQFTPLET
MINIDLLRTGDAWESGNYNFHTKPTNLAYHWQSQRHTGSCHPTVAPLVER
GQGTNIQSVNCWQWGDRNNPSSASTRVSNIHIGYSFPEWQIHYSTGGPVI
NPGSAFSQAPWGSTTEGTRLTQGASEKAIYDWSHGDDQPGARETWWQNNQ
HVTGQTDWAPKNAHTSELNNNVPAATHFWKNSYHNTFSPFTAVDDHGPQY
PWGAIWGKYPDTTHKPMMSAHAPFLLHGPPGQLFVKLAPNYTDTLDNGGV
THPRIVTYGTFWWSGQLIFKGKLRTPRQWNTYNLPSLDKRETMKNTVPNE
VGHFELPYMPGRCLPNYTL

Exemplary cutavirus VP2 capsid polypeptide sequences may be or comprise a coding sequence according to SEQ ID NO: 115.

ATGAGCGAGCCCGCCAACGACACCAACGAGCAGCCCGACAACAGCCCCGT
GGAGCAGGGCGCCGGCCAGATCGGCGGCGGCGGCGGCGGCGGCGGCAGCG
GCGTGGGCCACAGCACCGGCGACTACAACAACCGCACCGAGTTCATCTAC
CACGGCGACGAGGTGACCATCATCTGCCACAGCACCCGCCTGGTGCACAT
CAACATGAGCGACCGCGAGGACTACATCATCTACGAGACCGACCGCGGCC
CCCTGTTCCCCACCACCCAGGACCTGCAGGGCCGCGACACCCTGAACGAC
AGCTACCACGCCAAGGTGGAGACCCCCTGGAAGCTGCTGCACGCCAACAG
CTGGGGCTGCTGGTTCAGCCCCGCCGACTTCCAGCAGATGATCACCACCT
GCCGCGACATCGCCCCCATCAAGATGCACCAGAAGATCGAGAACATCGTG
ATCAAGACCGTGAGCAAGACCGGCACCGGCGAGACCGAGACCACCAACTA
CAACAACGACCTGACCGCCCTGCTGCAGATCGCCCAGGACAACAGCAACC
TGCTGCCCTGGGCCGCCGACAACTTCTACATCGACAGCGTGGGCTACGTG
CCCTGGCGCGCCTGCAAGCTGCCCACCTACTGCTACCACGTGGACACCTG
GAACACCATCGACATCAACCAGGCCGACACCCCCAACCAGTGGCGCGAGA
TCAAGAAGGGCATCCAGTGGGACAACATCCAGTTCACCCCCCTGGAGACC
ATGATCAACATCGACCTGCTGCGCACCGGCGACGCCTGGGAGAGCGGCAA
CTACAACTTCCACACCAAGCCCACCAACCTGGCCTACCACTGGCAGAGCC
AGCGCCACACCGGCAGCTGCCACCCCACCGTGGCCCCCCTGGTGGAGCGC
GGCCAGGGCACCAACATCCAGAGCGTGAACTGCTGGCAGTGGGGCGACCG
CAACAACCCCAGCAGCGCCAGCACCCGCGTGAGCAACATCCACATCGGCT
ACAGCTTCCCCGAGTGGCAGATCCACTACAGCACCGGCGGCCCCGTGATC
AACCCCGGCAGCGCCTTCAGCCAGGCCCCCTGGGGCAGCACCACCGAGGG
CACCCGCCTGACCCAGGGCGCCAGCGAGAAGGCCATCTACGACTGGAGCC
ACGGCGACGACCAGCCCGGCGCCCGCGAGACCTGGTGGCAGAACAACCAG
CACGTGACCGGCCAGACCGACTGGGCCCCCAAGAACGCCCACACCAGCGA
GCTGAACAACAACGTGCCCGCCGCCACCCACTTCTGGAAGAACAGCTACC
ACAACACCTTCAGCCCCTTCACCGCCGTGGACGACCACGGCCCCCAGTAC
CCCTGGGGCGCCATCTGGGGCAAGTACCCCGACACCACCCACAAGCCCAT
GATGAGCGCCCACGCCCCCTTCCTGCTGCACGGCCCCCCCGGCCAGCTGT
TCGTGAAGCTGGCCCCCAACTACACCGACACCCTGGACAACGGCGGCGTG
ACCCACCCCCGCATCGTGACCTACGGCACCTTCTGGTGGAGCGGCCAGCT
GATCTTCAAGGGCAAGCTGCGCACCCCCCGCCAGTGGAACACCTACAACC
TGCCCAGCCTGGACAAGCGCGAGACCATGAAGAACACCGTGCCCAACGAG
GTGGGCCACTTCGAGCTGCCCTACATGCCCGGCCGCTGCCTGCCCAACTA
CACCCTG

### Exemplary Feline Panleukopenia Virus (FPV) VP2 Sequences

Exemplary feline panleukopenia virus VP2 capsid polypeptide sequences may be or comprise a polypeptide sequence according to SEQ ID NO: 116.

MSDGAVQPDGGQPAVRNERATGSGNGSGGGGGGGSGGVGISTGTFNNQTE
FKFLENGWVEITANSSRLVHLNMPESENYKRVVVNNMDKTAVKGNMALDD
IHVQIVTPWSLVDANAWGVWFNPGDWQLIVNTMSELHLVSFEQEIFNVVL
KTVSESATQPPTKVYNNDLTASLMVALDSNNTMPFTPAAMRSETLGFYPW
KPTIPTPWRYYFQWDRTLIPSHTGTSGTPTNIYHGTDPDDVQFYTIENSV
PVHLLRTGDEFATGTFFFDCKPCRLTHTWQTNRALGLPPFLNSLPQSEGA
TNFGDIGVQQDKRRGVTQMGNTNYITEATIMRPAEVGYSAPYYSFEASTQ
GPFKTPIAAGRGGAQTDENQAADGDPRYAFGRQHGQKTTTTGETPERFTY
IAHQDTGRYPEGDWIQNINFNLPVTNDNVLLPTDPIGGKTGINYTNIFNT
YGPLTALNNVPPVYPNGQIWDKEFDTDLKPRLHVNAPFVCQNNCPGQLFV
KVAPNLTNEYDPDASANMSRIVTYSDFWWKGKLVFKAKLRASHTWNPIQQ
MSINVDNQFNYVPSNIGAMKIVYEKSQLAPRKLY

### Exemplary Tusavirus (TuV) VP2 Sequences

Exemplary tusavirus VP2 capsid polypeptide sequences may be or comprise a polypeptide sequence according to SEQ ID NO: 117.

MAASSSDSGPSSSGGGARAGGVGVSTGDFDNTTLWDFHEDGTATITCNST
RLVHLTRPDSLDYKIIPTQNNTAVQTVGHMMDDDNHTQVLTPWSLVDCNA
WGVWLSPHDWQHIMNIGEELELLSLEQEVFNVTLKTATETGPPESRITMY
NNDLTAVMMITTDTNNQLPYTPAAIRSETLGFYPWRPTVVPRWRYYFDWD
RFLSVTSSSDQSTSIINHSSTQSAIGQFFVIETQLPIALLRTGDSYATGG
YKFDCNKVNLGRHWQTTRSLGLPPKIEPPTSESALGTINQNARLGWRWGI
NDVHETNVVRPCTAGYNHPEWFYTHTLEGPAIDPAPPTSIPSNWGGGTPP
DTRASSHNQQRITYNYNHGNKDENLNNFSLNPNIELGSIINQGNFLSYEG
NGQQINTTAGVGKNGETATSDPNLVRYMPNTYGVYTAVDHQGPVYPHGQI
WDKQIHTDKKPELHCLAPFTCKNNPPGQMFVRIAPNLTDTFNATPTFSEI
ITYADFWWKGTLKMKIKLRPPHQWNIATVLGAAVNIGDAARFVPNRLGQL
EFPVINGRIVPSTVY

In some embodiments, a protoparvovirus capsid polypeptide comprises one or more of structural proteins of a protoparvovirus variant VP1 capsid polypeptide and/or VP2 capsid polypeptide. VP2 capsid polypeptide may be present in excess of VP1 (e.g., in ratio of VP2 capsid polypeptide to VP1 capsid polypeptide is 25:1, 20:1, 15:1, 10:1, 5:1).

iv. Expression Control Sequences

In some embodiments, a construct comprises an expression control sequence. In some embodiments, an expression control sequence comprises or is a promoter. The term “expression control sequence” or “promoter” refers to a DNA sequence recognized by enzymes/proteins that can promote and/or initiate transcription of an operably linked coding sequence. In some embodiments, a construct encoding a protoparvovirus variant VP1 capsid polypeptide can include a promoter and/or an enhancer. For example, a promoter typically refers to, e.g., a nucleotide sequence to which an RNA polymerase and/or any associated factor binds and from which it can initiate transcription. Thus, in some embodiments, a construct comprises a promoter operably linked to a non-limiting example promoter described herein. Additional examples of promoters are known in the art.

In some embodiments, a promoter comprises: (a) an immediate early promoter of an animal DNA virus, (b) an immediate early promoter of an insect virus, or (c) a host cell promoter. In some embodiments, a promoter is a polyhedrin (polh) promoter or an Immediately early 1 gene (IE-1) promoter. In some embodiments, a nucleotide sequence comprising at least one replication protein of an AAV (e.g., AAV2) comprises a nucleotide sequence encoding Rep52 and/or Rep78.

In some embodiments, an expression control sequence is a polyhedrin promoter, a P10 promoter, a CMV-b-actin promoter, an OpiE1 promoter, a JeT promoter, a Ubiquitin C promoter, or a truncated CMV enhancer and promoter. An exemplary polyhedrin promoter sequence may be or comprise a sequence according to SEQ ID NO: 118. An exemplary CMV-b-actin promoter sequence may be or comprise a sequence according to SEQ ID NO: 119. An exemplary OpiE1 promoter sequence may be or comprise a sequence according to SEQ ID NO: 120. An exemplary P10 promoter sequence may be or comprise a sequence according to SEQ ID NO: 121.

### Exemplary Polyhedrin Promoter Sequence (SEQ ID NO: 118)

CATGGAGATAATTAAAATGATAACCATCTCGCAAATAAATAAGTATTTTA
CTGTTTTCGTAACAGTTTTGTAATAAAAAAACCTATAAA
Exemplary CMV-b-actin promoter sequence
(SEQ ID NO: 119)
GGTACCTCTGGTCGTTACATAACTTACGGTAAATGGCCCGCCTGGCTGAC
CGCCCAACGACCCCGCCCATTGACGTCAATAATGACGTATGTTCCCATAG
TAACGCCAATAGGGACTTTCCATTGACGTCAATGGGTGGAGTATTTACGG
TAAACTGCCCACTTGGCAGTACATCAAGTGTATCATATGCCAAGTACGCC
CCCTATTGACGTCAATGACGGTAAATGGCCCGCCTGGCATTATGCCCAGT
ACATGACCTTATGGGACTTTCCTACTTGGCAGTACATCTACTCGAGGCCA
CGTTCTGCTTCACTCTCCCCATCTCCCCCCCCTCCCCACCCCCAATTTTG
TATTTATTTATTTTTTAATTATTTTGTGCAGCGATGGGGGCGGGGGGGGG
GGGGGGGCGCGCGCCAGGCGGGGCGGGGCGGGGCGAGGGGCGGGGCGGGG
CGAGGCGGAGAGGTGCGGCGGCAGCCAATCAGAGCGGCGCGCTCCGAAAG
TTTCCTTTTATGGCGAGGCGGCGGCGGCGGCGGCCCTATAAAAAGCGAAG
CGCGCGGCGGGCGGGAGCGGGATCAGCCAC
Exemplary OpiEl promoter sequence
(SEQ ID NO: 120)
GCGAAACACGCACGGCGCGCGCACGCAGCTTAGCACAAACGCGTCGTTGC
ACGCGCCCACCGCTAACCGCAGGCCAATCGGTCGGCCGGCCTCATATCCG
CTCACCAGCCGCGTCCTATCGGGCGCGGCTTCCGCGCCCATTTTGAATAA
ATAAACGATAACGCCGTTGGTGGCGTGAGGCATGTAAAAGGTTACATCAT
TATCTTGTTCGCCATCCGGTTGGTATAAATAGACGTTCATGTTGGTTTTT
GTTTCAGTTGCAAGTTGGCTGCGGCGCGCGCAGCACCTTTGC
Exemplary P10 promoter sequence
(SEQ ID NO: 121)
GACCTTTAATTCAACCCAACACAATATATTATAGTTAAATAAGAATTATT
ATCAAATCATTTGTATATTAATTAAAATACTATACTGTAAATTACATTTT
ATTTACAATC
Exemplary JeT promoter sequence
(SEQ ID NO: 157)
GGGCGGAGTTAGGGCGGAGCCAATCAGCGTGCGCCGTTCCGAAAGTTGCC
TTTTATGGCTGGGCGGAGAATGGGCGGTGAACGCCGATGATTATATAAGG
ACGCGCCGGGTGTGGCACAGCTAGTTCCGTCGCAGCCGGGATTTGGGTCG
CGGTTCTTGTTTGTGGATCCCTGTGATCGTCACTTGACA
Exemplary Ubiquitin C promoter sequence
(SEQ ID NO: 158)
GGCCTCCGCGCCGGGTTTTGGCGCCTCCCGCGGGCGCCCCCCTCCTCA
CGGCGAGCGCTGCCACGTCAGACGAAGGGCGCAGGAGCGTTCCTGATCCT
TCCGCCCGGACGCTCAGGACAGCGGCCCGCTGCTCATAAGACTCGGCCTT
AGAACCCCAGTATCAGCAGAAGGACATTTTAGGACGGGACTTGGGTGACT
CTAGGGCACTGGTTTTCTTTCCAGAGAGCGGAACAGGCGAGGAAAAGTAG
TCCCTTCTCGGCGATTCTGCGGAGGGATCTCCGTGGGGCGGTGAACGCCG
ATGATTATATAAGGACGCGCCGGGTGTGGCACAGCTAGTTCCGTCGCAGC
CGGGATTTGGGTCGCGGTTCTTGTTTGTGGATCGCTGTGATCGTCACTTG
GT
Exemplary truncated CMV enhancer and promoter
(SEQ ID NO: 159)
GGTAAACTGCCCACTTGGCAGTACATCAAGTGTATCATATGCCAAGTAC
GCCCCCTATTGACGTCAATGACGGTAAATGGCCCGCCTGGCATTATGCCC
AGTACATGACCTTATGGGACTTTCCTACTTGGCAGTACATCTACGTATTA
GTCATCGCTATTACCATGGTGATGCGGTTTTGGCAGTACATCAATGGGCG
TGGATAGCGGTTTGACTCACGGGGATTTCCAAGTCTCCACCCCATTGACG
TCAATGGGAGTTTGTTTTGGCACCAAAATCAACGGGACTTTCCAAAATGT
CGTAACAACTCCGCCCCATTGACGCAAATGGGCGGTAGGCGTGTACGGTG
GGAGGTCTATATAAGCAGAGCTCTCTG

v. Untranslated Regions (UTRs)

In some embodiments, any constructs described herein can include one or more untranslated regions. In some embodiments, a construct can include a 5′ UTR and/or a 3′ UTR sequence. In some embodiments, if more than one UTR is present, UTRs may come from a single gene or more than one gene.

As is understood by those of skill in the art, an untranslated region (UTR) of a gene is transcribed but not translated. In some embodiments, a 5′ UTR sequence starts at a transcription start site and continues to a translation initiation sequence but does not include that translation initiation sequence. In some embodiments, a 3′ UTR starts immediately following a stop codon and continues until a transcriptional termination signal. Without wishing to be bound by any particular theory, there is a growing body of evidence regarding regulatory roles played by UTRs in terms of stability of nucleic acid molecule and translation. In some embodiments, regulatory features of a UTR can be incorporated into any technologies (e.g., constructs, compositions, kits, or methods) as described herein to, e.g., enhance stability of a protein.

For example, in some embodiments, a 5′ UTR sequence is included in any constructs described herein. Non-limiting examples of 5′ UTR sequences including those from the following genes: albumin, serum amyloid A, Apolipoprotein A/B/E, transferrin, alpha fetoprotein, erythropoietin, and Factor VIII, can be used to enhance expression of a nucleic acid molecule, such as a mRNA. In some embodiments, 5′ UTR sequences have also been known, e.g., to form secondary structures that are involved in elongation factor binding.

In some embodiments, a 5′ UTR sequence from an mRNA that is transcribed by a cell can be included in any technologies (e.g., constructs, compositions, kits, and methods) described herein.

Among other things, the present example recognizes that selection of a 5′ UTR sequence can improve production of a protoparvovirus VP1 capsid polypeptide. Among other things, the present example recognizes that selection of a 5′ UTR sequence can reduce toxicity of a VP1 capsid polypeptide. In some embodiments, a 5′UTR is a stretch of nucleotides between an expression control sequence and a VP1 capsid coding sequence (referred to herein as “a nucleotide spacer sequence”).

In some embodiments, a nucleotide spacer sequence has a length of about 1 nucleotide. In some embodiments, a nucleotide spacer sequence has a length of about 5 nucleotides. In some embodiments, a nucleotide spacer sequence has a length of about 10 nucleotides. In some embodiments, a nucleotide spacer sequence has a length of about 20 nucleotides. In some embodiments, a nucleotide spacer sequence has a length of about 30 nucleotides. In some embodiments, a nucleotide spacer sequence has a length of about 40 nucleotides. In some embodiments, a nucleotide spacer sequence has a length of about 50 nucleotides. In some embodiments, a nucleotide spacer sequence has a length of about 60 nucleotides. In some embodiments, a nucleotide spacer sequence has a length of about 70 nucleotides. In some embodiments, a nucleotide spacer sequence has a length of about 80 nucleotides. In some embodiments, a nucleotide spacer sequence has a length of about 90 nucleotides. In some embodiments, a nucleotide spacer sequence has a length of about 100 nucleotides.

In some embodiments, a nucleotide spacer sequence has a length from about 1 to about 100 nucleotides. In some embodiments, a nucleotide spacer sequence has a length from about 1 to about 75 nucleotides. In some embodiments, a nucleotide spacer sequence has a length from about 10 to about 100 nucleotides. In some embodiments, a nucleotide spacer sequence has a length from about 1 to about 50 nucleotides. In some embodiments, a nucleotide spacer sequence has a length from about 1 to about 60 nucleotides. In some embodiments, a nucleotide spacer sequence has a length from about 30 to about 60 nucleotides. In some embodiments, a nucleotide spacer sequence has a length from about 1 to about 80 nucleotides. In some embodiments, a nucleotide spacer sequence has a length from about 1 to about 55 nucleotides. In some embodiments, a nucleotide spacer sequence has a length from about 10 to about 70 nucleotides. In some embodiments, a nucleotide spacer sequence has a length from about 1 to about 90 nucleotides. In some embodiments, a nucleotide spacer sequence has a length from about 1 to about 65 nucleotides. In some embodiments, a nucleotide spacer sequence has a length from about 45 nucleotides. In some embodiments, a nucleotide spacer sequence has a length from about 20 to about 80 nucleotides. In some embodiments, a nucleotide spacer sequence has a length from about 1 to about 75 nucleotides. In some embodiments, a nucleotide spacer sequence has a length from about 40 to about 80 nucleotides.

In some embodiments, there is no nucleotide spacer sequence.

In some embodiments, a 5′ UTR sequence comprises a viral 5′UTR sequence according to SEQ ID NO: 122. In some embodiments, a 5′ UTR sequence comprises a nucleotide spacer sequence according to SEQ ID NO: 123. In some embodiments, a 5′ UTR sequence comprises a nucleotide spacer sequence that does not comprise an alternative translation initiation sequence according to SEQ ID NO: 124.

Exemplary 5′ viral UTR sequence
(SEQ ID NO: 122)
CTCGACGAAGACTTGATCACCCGGGGGATCCCCTGTTAAG
Exemplary nucleotide spacer sequence 1
(SEQ ID NO: 123)
ATTCCGGATTATTCATACCGTCCCACCATCGGGCGCGGATCT
Exemplary nucleotide spacer sequence 2
(SEQ ID NO: 124)
ACTCCGGACTACTGATACCGTCCCACTTTCGGGCGCTTACCT

In some embodiments, 3′ UTRs are known to have stretches of adenosines and uridines embedded in them. These AU-rich signatures are particularly prevalent in genes with high rates of turnover. Based on their sequence features and functional properties, AU-rich elements (AREs) can be separated into three classes (Chen et al., Mol. Cell. Biol. 15:5777-5788, 1995; Chen et al., Mol. Cell Biol. 15:2010-2018, 1995, each of which is incorporated in its entirety herein by reference): Class I AREs contain several dispersed copies of an AUUUA motif within U-rich regions. For example, c-Myc and MyOD mRNAs contain class I AREs. Class II AREs possess two or more overlapping UUAUUUA(U/A)(U/A) nonamers. GM-CSF and TNF-alpha mRNAs are examples that contain class II AREs. Class III AREs are less well defined. These U-rich regions do not contain an AUUUA motif. Two well-studied examples of this class are c-Jun and myogenin mRNAs.

Most proteins binding to AREs are known to destabilize a messenger, whereas members of the ELAV family, most notably HuR, have been documented to increase stability of mRNA. HuR binds to AREs of all three classes. Engineering HuR specific binding sites into a 3′ UTR of nucleic acid molecules will lead to HuR binding and thus, stabilization of a message in vivo.

In some embodiments, introduction, removal, or modification of 3′ UTR AREs can be used to modulate stability of an mRNA encoding a protein. In some embodiments, AREs can be removed or mutated to increase intracellular stability and thus increase translation and production of a protein.

In some embodiments, a UTR sequence is at least 85%, 90%, 95%, 98% or 99% identical to any UTR sequence disclosed herein (e.g., SEQ ID NOs: 122-124)

vi. Kozak Consensus Sequences

In some embodiments, a construct of the present disclosure comprises one or more Kozak consensus sequences (also herein to as Kozak consensus sequences). In some embodiments, natural 5′ UTRs include a sequence that plays a role in translation initiation. For example, in some embodiments, they harbor signatures like Kozak sequences, which are commonly known to be involved in a process by which a ribosome initiates translation of many genes. Kozak sequences generally have a consensus sequence CCR(A/G)CCATGG, where R is a purine (A or G) three bases upstream of a translation initiation sequence (ATG), which is followed by another “G”. In some embodiments, Kozak sequences may be included in synthetic or additional sequence elements, such as cloning sites.

vii. Polyadenylation Sequences

In some embodiments, a construct of the present disclosure may comprise at least one poly(A) sequence. Most nascent eukaryotic mRNA possesses a poly(A) tail at its 3′ end which is added during a complex process that includes cleavage of a primary transcript and a coupled polyadenylation reaction (see, e.g., Proudfoot et al., Cell 108:501-512, 2002, the contents of which are hereby incorporated by reference herein in its entirety). A poly(A) tail confers mRNA stability and transferability (see, e.g., Molecular Biology of the Cell, Third Edition by B. Alberts et al., Garland Publishing, 1994, the contents of which are hereby incorporated by reference herein in its entirety). In some embodiments, a poly(A) sequence is positioned 3′ to a nucleic acid sequence encoding a transgene. In some embodiments, a poly(A) sequence is positioned 3′ to a VP1 capsid coding sequence encoding a protoparvovirus variant VP1 capsid polypeptide.

In some embodiments, polyadenylation refers to a covalent linkage of a polyadenylyl moiety, or its modified variant, to a messenger RNA molecule. In eukaryotic organisms, most messenger RNA (mRNA) molecules are polyadenylated at a 3′ end. In some embodiments, a 3′ poly(A) tail is a long sequence of adenine nucleotides (often several hundred) added to pre-mRNA through enzymatic action, polyadenylate polymerase. In higher eukaryotes, a poly(A) tail is added onto transcripts that contain a specific sequence, a polyadenylation signal. In some embodiments, a poly(A) tail and a protein bound to it aid in protecting mRNA from degradation by exonucleases. As will be understood to those of skill in the art, polyadenylation is also important for transcription termination, export of mRNA from a cell's nucleus, and translation. Polyadenylation occurs in a cell nucleus immediately after transcription of DNA into RNA, but additionally can also occur later in cytoplasm. After transcription has been terminated, an mRNA chain is cleaved through action of an endonuclease complex associated with RNA polymerase. A cleavage site is usually characterized by presence of a base sequence AAUAAA near a given cleavage site. After an mRNA has been cleaved, adenosine residues are added to a free 3′ end at a cleavage site.

In some embodiments, a poly(A) signal sequence is a sequence that triggers endonuclease cleavage of an mRNA and addition of a series of adenosines to the 3′ end of a cleaved mRNA. A “poly(A)” portion refers to a series of adenosines attached by polyadenylation to an mRNA. In some embodiments of for the present disclosure, such as, e.g., transient expression, a poly A is between 50 and 5000, preferably greater than 64, more preferably greater than 100, most preferably greater than 300 or 400. Poly (A) sequences can be modified chemically or enzymatically to modulate mRNA functionality such as localization, stability or efficiency of translation.

There are several poly(A) signal sequences that can be used, including those derived from bovine growth hormone (bgh) (Woychik et al., Proc. Natl. Acad. Sci. U.S.A. 81(13): 3944-3948, 1984; U.S. Pat. No. 5,122,458; Yew et al., Human Gene Ther. 8(5): 575-584, 1997; Xu et al., Human Gene Ther. 12(5): 563-573, 2001; Xu et al., Gene Ther. 8:1323-1332, 2001; Wu et al., Mol. Ther. 16(2): 280-289, 2008; Gray et al., Human Gene Ther: 22:1143-1153, 2011; Choi et al., Mol. Brain 7:17, 2014, each of which is incorporated in its entirety herein by reference), mouse-β-globin, mouse-α-globin (Orkin et al., EMBO J. 4(2): 453-456, 1985; Thein et al., Blood 71(2): 313-319, 1988, each which is incorporated in its entirety herein by reference), human collagen, polyoma virus (Batt et al., Mol. Cell Biol. 15(9): 4783-4790, 1995, each of which is incorporated in its entirety herein by reference), Herpes simplex virus thymidine kinase gene (HSV TK), IgG heavy-chain gene polyadenylation signal (US 2006/0040354, which is incorporated in its entirety herein by reference), human growth hormone (hGH) (Szymanski et al., Mol. Therapy 15(7): 1340-1347, 2007; Ostegaard et al., Proc. Natl. Acad. Sci. U.S.A. 102(8): 2952-2957, 2005, each of which is incorporated in its entirety herein by reference), synthetic poly A (Levitt et al., Genes Dev. 3(7): 1019-1025, 1989; Yew et al., Human Gene Ther. 8(5): 575-584, 1997; Ostegaard et al., Proc. Natl. Acad. Sci. U.S.A. 102(8): 2952-2957, 2005; Choi et al., Mol. Brain 7:17, 2014, each of which is incorporated in its entirety herein by reference), HIV-1 upstream poly(A) enhancer (Schambach et al., Mol. Ther. 15(6): 1167-1173, 2007, each of which is incorporated in its entirety herein by reference), adenovirus (L3) upstream poly(A) enhancer (Schambach et al., Mol. Ther. 15(6): 1167-1173, 2007, which is incorporated in its entirety herein by reference), hTHGB upstream poly(A) enhancer (Schambach et al., Mol. Ther. 15(6): 1167-1173, 2007), hC2 upstream poly(A) enhancer (Schambach et al., Mol. Ther. 15(6): 1167-1173, 2007), the group consisting of SV40 poly(A) signal sequence, such as the SV40 late and early poly(A) signal sequence (Schek et al., Mol. Cell Biol. 12(12): 5386-5393, 1992; Choi et al., Mol. Brain 7:17, 2014; Schambach et al., Mol. Ther. 15(6): 1167-1173, 2007, each of which is incorporated in its entirety herein by reference). The contents of each of these references are incorporated herein by reference in its entirety.

In some embodiments, a poly(A) signal sequence can be a sequence AATAAA. In some embodiments, an AATAAA sequence may be substituted with other hexanucleotide sequences with homology to AATAAA which are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414, which is incorporated in its entirety herein by reference).

In some embodiments, a poly(A) signal sequence can be a synthetic polyadenylation site (see, e.g., the pCl-neo expression construct of Promega which is based on Levitt et al, Genes Dev. 3(7): 1019-1025, 1989, which is incorporated in its entirety herein by reference). In some embodiments, a poly(A) signal sequence is a polyadenylation signal of soluble neuropilin-1 (sNRP) (see, e.g., WO 05/073384, which is incorporated in its entirety herein by reference). In some embodiments, a poly(A) sequence is a bovine growth hormone poly(A) sequence. Additional examples of poly(A) signal sequences are known in the art.

In some embodiments, a polyA sequence is at least 85%, 90%, 95%, 98% or 99% identical to the poly A sequence of SEQ ID NO: 125.

By way of non-limiting example, a polyadenylation sequence may be or comprise a sequence according to SEQ ID NO: 125.

Exemplary SV40 PolyA Sequence
(SEQ ID NO: 125)
TTGTTTATTGCAGCTTATAATGGTTACAAATAAAGCAATAGCATCACAAA
TTTCACAAATAAAGCATTTTTTTCACTGCATTCTAGTTGTGGTTTGTCCA
AACTCATCAATGTATCTTATCATGTCTGGATC
viii. Enhancers and 5′ cap

In some instances, a construct can include an expression control sequence and/or an enhancer sequence. In some embodiments, an enhancer is a nucleotide sequence that can increase a level of transcription of a nucleic acid encoding a polypeptide of interest (e.g., a protoparvovirus variant VP1 capsid polypeptide). In some embodiments, enhancer sequences (50-1500 base pairs in length) generally increase a level of transcription by providing additional binding sites for transcription-associated proteins (e.g., transcription factors). In some embodiments, an enhancer sequence is found within an intronic sequence. Unlike promoter sequences, enhancer sequences can act at much larger distance away from a transcription start site (e.g., as compared to a promoter). Non-limiting examples of enhancers include a RSV enhancer, a CMV enhancer, and a SV40 enhancer. An example of a CMV enhancer is described in, e.g., Boshart et al., Cell 41(2): 521-530, 1985, which is incorporated in its entirety herein by reference.

As described herein, a 5′ cap (also termed an RNA cap, an RNA 7-methylguanosine cap or an RNA m.sup.7G cap) is a modified guanine nucleotide that has been added to a “front” or 5′ end of a eukaryotic messenger RNA shortly after a start of transcription. In some embodiments, a 5′ cap consists of a terminal group which is linked to a first transcribed nucleotide. Its presence is critical for recognition by a ribosome and protection from RNases. Cap addition is coupled to transcription, and occurs co-transcriptionally, such that each influences the other. Shortly after start of transcription, a 5′ end of an mRNA being synthesized is bound by a cap-synthesizing complex associated with RNA polymerase. This enzymatic complex catalyzes a chemical reactions that are required for mRNA capping. Synthesis proceeds as a multi-step biochemical reaction. A capping moiety can be modified to modulate functionality of mRNA such as its stability or efficiency of translation.

ix. Exemplary Capsid Construct Sequences

In some embodiments, the present disclosure provides technologies (e.g., compositions, systems, particles, comprising protoparvovirus-related constructs). In some embodiments, such technologies comprise a single construct. In some embodiments, such technologies comprise multiple constructs. In some embodiments, the present disclosure provides compositions or systems comprising multiple virions each comprised of a single construct as described herein. In some embodiments, a single construct may deliver a polynucleotide that encodes a functional (e.g., wild type or otherwise functional, e.g., codon optimized) copy of a protoparvovirus variant VP1 gene. In some embodiments, a construct is or comprises a protoparvovirus-related construct.

In some embodiments, a single construct composition or system may comprise any or all of the exemplary construct components described herein. In some embodiments, an exemplary single construct is at least 85%, 90%, 95%, 98% or 99% identical to the sequences described herein. One skilled in the art would recognize that constructs may undergo additional modifications including codon-optimization, introduction of novel but functionally equivalent (e.g., silent mutations), addition of reporter sequences, and/or other routine modification.

Among other things, the present disclosure includes exemplary reference and protoparvovirus variant VP1 capsid polypeptide construct sequences described herein as shown in Table 4.

Table 4 shows exemplary constructs described herein.

TABLE 4

| Exemplary | | SEQ ID |
|---|---|---|
| Construct | Sequence | NO: |
| Exemplary | CATGGAGATAATTAAAATGATAACCATCTCGCAAATAAATAA | SEQ ID |
| CPV | GTATTTTACTGTTTTCGTAACAGTTTTGTAATAAAAAAACCT | NO: 126 |
| Construct 1 | ATAAAATTCCGGATTATTCATACCGTCCCACCATCGGGCGCG | |
| comprising | GATCTCCTGTTAAGCTGGCACCTCCGGCAAAGAGAGCCAG | |
| a proto- | GAGAGGATATAAATATCTTGGGCCTGGGAACAGTCTTGACC | |
| parvovirus | AAGGAGAACCAACTAACCCTTCTGACGCCGCTGCAAAAGA | |
| variant | ACACGACGAAGCTTACGCTGCTTATCTTCGCTCTGGTAAAA | |
| VP1 capsid | ACCCATACTTATATTTCTCGCCAGCAGATCAACGCTTTATAG | |
| coding | ATCAAACTAAGGACGCTAAAGATTGGGGGGGGAAAATAGG | |
| sequence | ACATTATTTTTTTAGAGCTAAAAAGGCAATTGCTCCAGTATT | |
| Ph-v5UTR- | AACTGATACACCAGATCATCCATCAACATCAAGACCAACAA | |
| CPV-VP1- | AACCAACTAAAAGAAGTAAACCACCACCTCATATTTTCATC | |
| CTG-Del- | AATCTTGCAAAAAAAAAAAAAGCCGGTGCAGGACAAGTA | |
| LVPPG | AAAAGAGACAATCTTGCACCAATGAGTGATGGAGCAGTTC | |
| | AACCAGACGGTGGTCAACCTGCTGTCAGAAATGAAAGAG | |
| | CTACAGGATCTGGGAACGGGTCTGGAGGCGGGGGTGGTGG | |
| | TGGTTCTGGGGGTGTGGGGATTTCTACGGGTACTTTCAATA | |
| | ATCAGACGGAATTTAAATTTTTGGAAAACGGATGGGTGGA | |
| | AATCACAGCAAACTCAAGCAGACTTGTACATTTAAATATGC | |
| | CAGAAAGTGAAAATTATAGAAGAGTGGTTGTAAATAATATG | |
| | GATAAAACTGCAGTTAACGGAAACATGGCTTTAGATGATAT | |
| | TCATGCACAAATTGTAACACCTTGGTCATTGGTTGATGCAA | |
| | ATGCTTGGGGAGTTTGGTTTAATCCAGGAGATTGGCAACTA | |
| | ATTGTTAATACTATGAGTGAGTTGCATTTAGTTAGTTTTGAA | |
| | CAAGAAATTTTTAATGTTGTTTTAAAGACTGTTTCAGAATC | |
| | TGCTACTCAGCCACCAACTAAAGTTTATAATAATGATTTAAC | |
| | TGCATCATTGATGGTTGCATTAGATAGTAATAATACTATGCC | |
| | ATTTACTCCAGCAGCTATGAGATCTGAGACATTGGGTTTTTA | |
| | TCCATGGAAACCAACCATACCAACTCCATGGAGATATTATTT | |
| | TCAATGGGATAGAACATTAATACCATCTCATACTGGAACTAG | |
| | TGGCACACCAACAAATATATACCATGGTACAGATCCAGATG | |
| | ATGTTCAATTTTATACTATTGAAAATTCTGTGCCAGTACACT | |
| | TACTAAGAACAGGTGATGAATTTGCTACAGGAACATTTTTT | |
| | TTTGATTGTAAACCATGTAGACTAACACATACATGGCAAAC | |
| | AAATAGAGCATTGGGCTTACCACCATTTCTAAATTCTTTGCC | |
| | TCAATCTGAAGGAGCTACTAACTTTGGTGATATAGGAGTTC | |
| | AACAAGATAAAAGACGTGGTGTAACTCAAATGGGAAATAC | |
| | AAACTATATTACTGAAGCTACTATTATGAGACCAGCTGAGG | |
| | TTGGTTATAGTGCACCATATTATTCTTTTGAGGCGTCTACAC | |
| | AAGGGCCATTTAAAACACCTATTGCAGCAGGACGGGGGGG | |
| | AGCGCAAACATATGAAAATCAAGCAGCAGATGGTGATCCA | |
| | AGATATGCATTTGGTAGACAACATGGTCAAAAAACTACCAC | |
| | AACAGGAGAAACACCTGAGAGATTTACATATATAGCACATC | |
| | AAGATACAGGAAGATATCCAGAAGGAGATTGGATTCAAAA | |
| | TATTAACTTTAACCTTCCTGTAACGAATGATAATGTATTGCT | |
| | ACCAACAGATCCAATTGGAGGTAAAACAGGAATTAACTATA | |
| | CTAATATATTTAATACTTATGGTCCTTTAACTGCATTAAATAA | |
| | TGTACCACCAGTTTATCCAAATGGTCAAATTTGGGATAAAG | |
| | AATTTGATACTGACTTAAAACCAAGACTTCATGTAAATGCA | |
| | CCATTTGTTTGTCAAAATAATTGTCCTGGTCAATTATTTGTA | |
| | AAAGTTGCGCCTAATTTAACAAATGAATATGATCCTGATGC | |
| | ATCTGCTAATATGTCAAGAATTGTAACTTACTCAGATTTTTG | |
| | GTGGAAAGGTAAATTAGTATTTAAAGCTAAACTAAGAGCCT | |
| | CTCATACTTGGAATCCAATTCAACAAATGAGTATTAATGTAG | |
| | ATAACCAATTTAACTATGTACCAAGTAATATTGGAGGTATGA | |
| | AAATTGTATATGAAAAATCTCAACTAGCACCTAGAAAATTAT | |
| | ATTAACTCGAGGCATGCGGTACCAAGCTTGTCGAGAAGTA | |
| | CTAGAGGATCATAATCAGCCATACCACATTTGTAGAGGTTTT | |
| | ACTTGCTTTAAAAAACCTCCCACACCTCCCCCTGAACCTGA | |
| | AACATAAAATGAATGCAATTGTTGTTGTTAACTTGTTTATTG | |
| | CAGCTTATAATGGTTACAAATAAAGCAATAGCATCACAAAT | |
| | TTCACAAATAAAGCATTTTTTTCACTGCATTCTAGTTGTGGT | |
| | TTGTCCAAACTCATCAATGTATCTTATCATGTCTGGATC | |
| Exemplary | CATGGAGATAATTAAAATGATAACCATCTCGCAAATAAATAA | SEQ ID |
| CPV | GTATTTTACTGTTTTCGTAACAGTTTTGTAATAAAAAAACCT | NO: 127 |
| Construct 2 | ATAAACTGGCACCTCCGGCAAAGAGAGCCAGGAGAGGATA | |
| comprising | TAAATATCTTGGGCCTGGGAACAGTCTTGACCAAGGAGAA | |
| a proto- | CCAACTAACCCTTCTGACGCCGCTGCAAAAGAACACGACG | |
| parvovirus | AAGCTTACGCTGCTTATCTTCGCTCTGGTAAAAACCCATAC | |
| variant | TTATATTTCTCGCCAGCAGATCAACGCTTTATAGATCAAACT | |
| VP1 capsid | AAGGACGCTAAAGATTGGGGGGGGAAAATAGGACATTATT | |
| coding | TTTTTAGAGCTAAAAAGGCAATTGCTCCAGTATTAACTGAT | |
| sequence | ACACCAGATCATCCATCAACATCAAGACCAACAAAACCAA | |
| Ph-CPV- | CTAAAAGAAGTAAACCACCACCTCATATTTTCATCAATCTT | |
| VP1-CTG- | GCAAAAAAAAAAAAAGCCGGTGCAGGACAAGTAAAAAGA | |
| Del-LVPPG | GACAATCTTGCACCAATGAGTGATGGAGCAGTTCAACCAG | |
| | ACGGTGGTCAACCTGCTGTCAGAAATGAAAGAGCTACAGG | |
| | ATCTGGGAACGGGTCTGGAGGCGGGGGTGGTGGTGGTTCT | |
| | GGGGGTGTGGGGATTTCTACGGGTACTTTCAATAATCAGAC | |
| | GGAATTTAAATTTTTGGAAAACGGATGGGTGGAAATCACA | |
| | GCAAACTCAAGCAGACTTGTACATTTAAATATGCCAGAAAG | |
| | TGAAAATTATAGAAGAGTGGTTGTAAATAATATGGATAAAA | |
| | CTGCAGTTAACGGAAACATGGCTTTAGATGATATTCATGCA | |
| | CAAATTGTAACACCTTGGTCATTGGTTGATGCAAATGCTTG | |
| | GGGAGTTTGGTTTAATCCAGGAGATTGGCAACTAATTGTTA | |
| | ATACTATGAGTGAGTTGCATTTAGTTAGTTTTGAACAAGAA | |
| | ATTTTTAATGTTGTTTTAAAGACTGTTTCAGAATCTGCTACT | |
| | CAGCCACCAACTAAAGTTTATAATAATGATTTAACTGCATCA | |
| | TTGATGGTTGCATTAGATAGTAATAATACTATGCCATTTACTC | |
| | CAGCAGCTATGAGATCTGAGACATTGGGTTTTTATCCATGG | |
| | AAACCAACCATACCAACTCCATGGAGATATTATTTTCAATG | |
| | GGATAGAACATTAATACCATCTCATACTGGAACTAGTGGCA | |
| | CACCAACAAATATATACCATGGTACAGATCCAGATGATGTTC | |
| | AATTTTATACTATTGAAAATTCTGTGCCAGTACACTTACTAA | |
| | GAACAGGTGATGAATTTGCTACAGGAACATTTTTTTTTGAT | |
| | TGTAAACCATGTAGACTAACACATACATGGCAAACAAATAG | |
| | AGCATTGGGCTTACCACCATTTCTAAATTCTTTGCCTCAATC | |
| | TGAAGGAGCTACTAACTTTGGTGATATAGGAGTTCAACAAG | |
| | ATAAAAGACGTGGTGTAACTCAAATGGGAAATACAAACTAT | |
| | ATTACTGAAGCTACTATTATGAGACCAGCTGAGGTTGGTTAT | |
| | AGTGCACCATATTATTCTTTTGAGGCGTCTACACAAGGGCC | |
| | ATTTAAAACACCTATTGCAGCAGGACGGGGGGGAGCGCAA | |
| | ACATATGAAAATCAAGCAGCAGATGGTGATCCAAGATATGC | |
| | ATTTGGTAGACAACATGGTCAAAAAACTACCACAACAGGA | |
| | GAAACACCTGAGAGATTTACATATATAGCACATCAAGATAC | |
| | AGGAAGATATCCAGAAGGAGATTGGATTCAAAATATTAACT | |
| | TTAACCTTCCTGTAACGAATGATAATGTATTGCTACCAACAG | |
| | ATCCAATTGGAGGTAAAACAGGAATTAACTATACTAATATAT | |
| | TTAATACTTATGGTCCTTTAACTGCATTAAATAATGTACCAC | |
| | CAGTTTATCCAAATGGTCAAATTTGGGATAAAGAATTTGAT | |
| | ACTGACTTAAAACCAAGACTTCATGTAAATGCACCATTTGT | |
| | TTGTCAAAATAATTGTCCTGGTCAATTATTTGTAAAAGTTGC | |
| | GCCTAATTTAACAAATGAATATGATCCTGATGCATCTGCTAA | |
| | TATGTCAAGAATTGTAACTTACTCAGATTTTTGGTGGAAAG | |
| | GTAAATTAGTATTTAAAGCTAAACTAAGAGCCTCTCATACTT | |
| | GGAATCCAATTCAACAAATGAGTATTAATGTAGATAACCAA | |
| | TTTAACTATGTACCAAGTAATATTGGAGGTATGAAAATTGTA | |
| | TATGAAAAATCTCAACTAGCACCTAGAAAATTATATTAACTC | |
| | GAGGCATGCGGTACCAAGCTTGTCGAGAAGTACTAGAGGA | |
| | TCATAATCAGCCATACCACATTTGTAGAGGTTTTACTTGCTT | |
| | TAAAAAACCTCCCACACCTCCCCCTGAACCTGAAACATAA | |
| | AATGAATGCAATTGTTGTTGTTAACTTGTTTATTGCAGCTTA | |
| | TAATGGTTACAAATAAAGCAATAGCATCACAAATTTCACAA | |
| | ATAAAGCATTTTTTTCACTGCATTCTAGTTGTGGTTTGTCCA | |
| | AACTCATCAATGTATCTTATCATGTCTGGATC | |
| Exemplary | CATGGAGATAATTAAAATGATAACCATCTCGCAAATAAATAA | SEQ ID |
| CPV | GTATTTTACTGTTTTCGTAACAGTTTTGTAATAAAAAAACCT | NO: 128 |
| Construct 3 | ATAAAACGGCACCTCCGGCAAAGAGAGCCAGGAGAGGAT | |
| comprising | ATAAATATCTTGGGCCTGGGAACAGTCTTGACCAAGGAGA | |
| a proto- | ACCAACTAACCCTTCTGACGCCGCTGCAAAAGAACACGAC | |
| parvovirus | GAAGCTTACGCTGCTTATCTTCGCTCTGGTAAAAACCCATA | |
| variant | CTTATATTTCTCGCCAGCAGATCAACGCTTTATAGATCAAAC | |
| VP1 capsid | TAAGGACGCTAAAGATTGGGGGGGGAAAATAGGACATTAT | |
| coding | TTTTTTAGAGCTAAAAAGGCAATTGCTCCAGTATTAACTGA | |
| sequence | TACACCAGATCATCCATCAACATCAAGACCAACAAAACCA | |
| Ph-CPV- | ACTAAAAGAAGTAAACCACCACCTCATATTTTCATCAATCT | |
| VP1-ACG- | TGCAAAAAAAAAAAAAGCCGGTGCAGGACAAGTAAAAAG | |
| Del-LVPPG | AGACAATCTTGCACCAATGAGTGATGGAGCAGTTCAACCA | |
| | GACGGTGGTCAACCTGCTGTCAGAAATGAAAGAGCTACAG | |
| | GATCTGGGAACGGGTCTGGAGGCGGGGGTGGTGGTGGTTC | |
| | TGGGGGTGTGGGGATTTCTACGGGTACTTTCAATAATCAGA | |
| | CGGAATTTAAATTTTTGGAAAACGGATGGGTGGAAATCAC | |
| | AGCAAACTCAAGCAGACTTGTACATTTAAATATGCCAGAAA | |
| | GTGAAAATTATAGAAGAGTGGTTGTAAATAATATGGATAAA | |
| | ACTGCAGTTAACGGAAACATGGCTTTAGATGATATTCATGC | |
| | ACAAATTGTAACACCTTGGTCATTGGTTGATGCAAATGCTT | |
| | GGGGAGTTTGGTTTAATCCAGGAGATTGGCAACTAATTGTT | |
| | AATACTATGAGTGAGTTGCATTTAGTTAGTTTTGAACAAGA | |
| | AATTTTTAATGTTGTTTTAAAGACTGTTTCAGAATCTGCTAC | |
| | TCAGCCACCAACTAAAGTTTATAATAATGATTTAACTGCATC | |
| | ATTGATGGTTGCATTAGATAGTAATAATACTATGCCATTTACT | |
| | CCAGCAGCTATGAGATCTGAGACATTGGGTTTTTATCCATG | |
| | GAAACCAACCATACCAACTCCATGGAGATATTATTTTCAAT | |
| | GGGATAGAACATTAATACCATCTCATACTGGAACTAGTGGC | |
| | ACACCAACAAATATATACCATGGTACAGATCCAGATGATGT | |
| | TCAATTTTATACTATTGAAAATTCTGTGCCAGTACACTTACT | |
| | AAGAACAGGTGATGAATTTGCTACAGGAACATTTTTTTTTG | |
| | ATTGTAAACCATGTAGACTAACACATACATGGCAAACAAAT | |
| | AGAGCATTGGGCTTACCACCATTTCTAAATTCTTTGCCTCA | |
| | ATCTGAAGGAGCTACTAACTTTGGTGATATAGGAGTTCAAC | |
| | AAGATAAAAGACGTGGTGTAACTCAAATGGGAAATACAAA | |
| | CTATATTACTGAAGCTACTATTATGAGACCAGCTGAGGTTGG | |
| | TTATAGTGCACCATATTATTCTTTTGAGGCGTCTACACAAGG | |
| | GCCATTTAAAACACCTATTGCAGCAGGACGGGGGGGAGCG | |
| | CAAACATATGAAAATCAAGCAGCAGATGGTGATCCAAGAT | |
| | ATGCATTTGGTAGACAACATGGTCAAAAAACTACCACAAC | |
| | AGGAGAAACACCTGAGAGATTTACATATATAGCACATCAAG | |
| | ATACAGGAAGATATCCAGAAGGAGATTGGATTCAAAATATT | |
| | AACTTTAACCTTCCTGTAACGAATGATAATGTATTGCTACCA | |
| | ACAGATCCAATTGGAGGTAAAACAGGAATTAACTATACTAA | |
| | TATATTTAATACTTATGGTCCTTTAACTGCATTAAATAATGTA | |
| | CCACCAGTTTATCCAAATGGTCAAATTTGGGATAAAGAATT | |
| | TGATACTGACTTAAAACCAAGACTTCATGTAAATGCACCAT | |
| | TTGTTTGTCAAAATAATTGTCCTGGTCAATTATTTGTAAAAG | |
| | TTGCGCCTAATTTAACAAATGAATATGATCCTGATGCATCTG | |
| | CTAATATGTCAAGAATTGTAACTTACTCAGATTTTTGGTGGA | |
| | AAGGTAAATTAGTATTTAAAGCTAAACTAAGAGCCTCTCAT | |
| | ACTTGGAATCCAATTCAACAAATGAGTATTAATGTAGATAA | |
| | CCAATTTAACTATGTACCAAGTAATATTGGAGGTATGAAAAT | |
| | TGTATATGAAAAATCTCAACTAGCACCTAGAAAATTATATTA | |
| | ACTCGAGGCATGCGGTACCAAGCTTGTCGAGAAGTACTAG | |
| | AGGATCATAATCAGCCATACCACATTTGTAGAGGTTTTACTT | |
| | GCTTTAAAAAACCTCCCACACCTCCCCCTGAACCTGAAAC | |
| | ATAAAATGAATGCAATTGTTGTTGTTAACTTGTTTATTGCAG | |
| | CTTATAATGGTTACAAATAAAGCAATAGCATCACAAATTTCA | |
| | CAAATAAAGCATTTTTTTCACTGCATTCTAGTTGTGGTTTGT | |
| | CCAAACTCATCAATGTATCTTATCATGTCTGGATC | |
| Exemplary | CATGGAGATAATTAAAATGATAACCATCTCGCAAATAAATAA | SEQ ID |
| CPV | GTATTTTACTGTTTTCGTAACAGTTTTGTAATAAAAAAACCT | NO: 129 |
| Construct 4 | ATAAATTGGCACCTCCGGCAAAGAGAGCCAGGAGAGGATA | |
| comprising | TAAATATCTTGGGCCTGGGAACAGTCTTGACCAAGGAGAA | |
| a proto- | CCAACTAACCCTTCTGACGCCGCTGCAAAAGAACACGACG | |
| parvovirus | AAGCTTACGCTGCTTATCTTCGCTCTGGTAAAAACCCATAC | |
| variant | TTATATTTCTCGCCAGCAGATCAACGCTTTATAGATCAAACT | |
| VP1 capsid | AAGGACGCTAAAGATTGGGGGGGGAAAATAGGACATTATT | |
| coding | TTTTTAGAGCTAAAAAGGCAATTGCTCCAGTATTAACTGAT | |
| sequence | ACACCAGATCATCCATCAACATCAAGACCAACAAAACCAA | |
| Ph-CPV- | CTAAAAGAAGTAAACCACCACCTCATATTTTCATCAATCTT | |
| VP1-TTG- | GCAAAAAAAAAAAAAGCCGGTGCAGGACAAGTAAAAAGA | |
| Del-LVPPG | GACAATCTTGCACCAATGAGTGATGGAGCAGTTCAACCAG | |
| | ACGGTGGTCAACCTGCTGTCAGAAATGAAAGAGCTACAGG | |
| | ATCTGGGAACGGGTCTGGAGGCGGGGGTGGTGGTGGTTCT | |
| | GGGGGTGTGGGGATTTCTACGGGTACTTTCAATAATCAGAC | |
| | GGAATTTAAATTTTTGGAAAACGGATGGGTGGAAATCACA | |
| | GCAAACTCAAGCAGACTTGTACATTTAAATATGCCAGAAAG | |
| | TGAAAATTATAGAAGAGTGGTTGTAAATAATATGGATAAAA | |
| | CTGCAGTTAACGGAAACATGGCTTTAGATGATATTCATGCA | |
| | CAAATTGTAACACCTTGGTCATTGGTTGATGCAAATGCTTG | |
| | GGGAGTTTGGTTTAATCCAGGAGATTGGCAACTAATTGTTA | |
| | ATACTATGAGTGAGTTGCATTTAGTTAGTTTTGAACAAGAA | |
| | ATTTTTAATGTTGTTTTAAAGACTGTTTCAGAATCTGCTACT | |
| | CAGCCACCAACTAAAGTTTATAATAATGATTTAACTGCATCA | |
| | TTGATGGTTGCATTAGATAGTAATAATACTATGCCATTTACTC | |
| | CAGCAGCTATGAGATCTGAGACATTGGGTTTTTATCCATGG | |
| | AAACCAACCATACCAACTCCATGGAGATATTATTTTCAATG | |
| | GGATAGAACATTAATACCATCTCATACTGGAACTAGTGGCA | |
| | CACCAACAAATATATACCATGGTACAGATCCAGATGATGTTC | |
| | AATTTTATACTATTGAAAATTCTGTGCCAGTACACTTACTAA | |
| | GAACAGGTGATGAATTTGCTACAGGAACATTTTTTTTTGAT | |
| | TGTAAACCATGTAGACTAACACATACATGGCAAACAAATAG | |
| | AGCATTGGGCTTACCACCATTTCTAAATTCTTTGCCTCAATC | |
| | TGAAGGAGCTACTAACTTTGGTGATATAGGAGTTCAACAAG | |
| | ATAAAAGACGTGGTGTAACTCAAATGGGAAATACAAACTAT | |
| | ATTACTGAAGCTACTATTATGAGACCAGCTGAGGTTGGTTAT | |
| | AGTGCACCATATTATTCTTTTGAGGCGTCTACACAAGGGCC | |
| | ATTTAAAACACCTATTGCAGCAGGACGGGGGGGAGCGCAA | |
| | ACATATGAAAATCAAGCAGCAGATGGTGATCCAAGATATGC | |
| | ATTTGGTAGACAACATGGTCAAAAAACTACCACAACAGGA | |
| | GAAACACCTGAGAGATTTACATATATAGCACATCAAGATAC | |
| | AGGAAGATATCCAGAAGGAGATTGGATTCAAAATATTAACT | |
| | TTAACCTTCCTGTAACGAATGATAATGTATTGCTACCAACAG | |
| | ATCCAATTGGAGGTAAAACAGGAATTAACTATACTAATATAT | |
| | TTAATACTTATGGTCCTTTAACTGCATTAAATAATGTACCAC | |
| | CAGTTTATCCAAATGGTCAAATTTGGGATAAAGAATTTGAT | |
| | ACTGACTTAAAACCAAGACTTCATGTAAATGCACCATTTGT | |
| | TTGTCAAAATAATTGTCCTGGTCAATTATTTGTAAAAGTTGC | |
| | GCCTAATTTAACAAATGAATATGATCCTGATGCATCTGCTAA | |
| | TATGTCAAGAATTGTAACTTACTCAGATTTTTGGTGGAAAG | |
| | GTAAATTAGTATTTAAAGCTAAACTAAGAGCCTCTCATACTT | |
| | GGAATCCAATTCAACAAATGAGTATTAATGTAGATAACCAA | |
| | TTTAACTATGTACCAAGTAATATTGGAGGTATGAAAATTGTA | |
| | TATGAAAAATCTCAACTAGCACCTAGAAAATTATATTAACTC | |
| | GAGGCATGCGGTACCAAGCTTGTCGAGAAGTACTAGAGGA | |
| | TCATAATCAGCCATACCACATTTGTAGAGGTTTTACTTGCTT | |
| | TAAAAAACCTCCCACACCTCCCCCTGAACCTGAAACATAA | |
| | AATGAATGCAATTGTTGTTGTTAACTTGTTTATTGCAGCTTA | |
| | TAATGGTTACAAATAAAGCAATAGCATCACAAATTTCACAA | |
| | ATAAAGCATTTTTTTCACTGCATTCTAGTTGTGGTTTGTCCA | |
| | AACTCATCAATGTATCTTATCATGTCTGGATC | |
| Exemplary | GACATTGATTATTGACTAGTTATTAATAGTAATCAATTACGG | SEQ ID |
| CPV | GGTCATTAGTTCATAGCCCATATATGGAGTTCCGCGTTACAT | NO: 130 |
| Construct 5 | AACTTACGGTAAATGGCCCGCCTGGCTGACCGCCCAACGA | |
| comprising | CCCCCGCCCATTGACGTCAATAATGACGTATGTTCCCATAGT | |
| a proto- | AACGCCAATAGGGACTTTCCATTGACGTCAATGGGTGGACT | |
| parvovirus | ATTTACGGTAAACTGCCCACTTGGCAGTACATCAAGTGTAT | |
| variant | CATATGCCAAGTACGCCCCCTATTGACGTCAATGACGGTAA | |
| VP1 capsid | ATGGCCCGCCTGGCATTATGCCCAGTACATGACCTTATGGG | |
| coding | ACTTTCCTACTTGGCAGTACATCTACGTATTAGTCATCGCTA | |
| sequence | TTACCATGGTGATGCGGTTTTGGCAGTACATCAATGGGCGT | |
| | GGATAGCGGTTTGACTCACGGGGATTTCCAAGTCTCCACCC | |
| | CATTGACGTCAATGGGAGTTTGTTTTGGCACCAAAATCAAC | |
| | GGGACTTTCCAAAATGTCGTAACAACTCCGCCCCATTGACG | |
| | CAAATGGGCGGTAGGCGTGTACGGTGGGAGGTCTATATAAG | |
| | CAGAGCTCTCTGGCTAACTAGAGAACCCACTGCTTACTGG | |
| | CTTATCGAAATTAATACGACTCACTATAGGGAGACCCAAGC | |
| | TTGGTACCGGACTCTAGAGGATCCGGTACTCGAGGAACTG | |
| | AAAAACCAGAAAGTTAACTGGTAAGTTTAGTCTTTTTGTCT | |
| | TTTATTTCAGGTCCCGGATCCGGTGGTGGTGCAAATCAAAG | |
| | AACTGCTCCTCAGTGGATGTTGCCTTTACTTCTAGGCCTGT | |
| | ACGGAAGTGTTACTTCTGCTCTAAAAGCTGCGGAATTGTAC | |
| | CCGCGGAAGCTTCCTAGGCCGCCACCATGGCCCCCCCCGC | |
| | CAAGCGCGCCCGCCGCGGCTACAAGTACCTGGGCCCCGGC | |
| | AACAGCCTGGACCAGGGCGAGCCCACCAACCCCAGCGAC | |
| | GCCGCCGCCAAGGAGCACGACGAGGCCTACGCCGCCTACC | |
| | TGCGCAGCGGCAAGAACCCCTACCTGTACTTCAGCCCCGC | |
| | CGACCAGCGCTTCATCGACCAGACCAAGGACGCCAAGGA | |
| | CTGGGGCGGCAAGATCGGCCACTACTTCTTCCGCGCCAAG | |
| | AAGGCCATCGCCCCCGTGCTGACCGACACCCCCGACCACC | |
| | CCAGCACCAGCCGCCCCACCAAGCCCACCAAGCGCAGCA | |
| | AGCCCCCCCCCCACATCTTCATCAACCTGGCCAAGAAGAA | |
| | GAAGGCCGGCGCCGGCCAGGTGAAGCGCGACAACCTGGC | |
| | CCCCATGAGCGACGGCGCCGTGCAGCCCGACGGCGGCCA | |
| | GCCCGCCGTGCGCAACGAGCGCGCCACCGGCAGCGGCAA | |
| | CGGCAGCGGCGGCGGCGGCGGCGGCGGCAGCGGCGGCGT | |
| | GGGCATCAGCACCGGCACCTTCAACAACCAGACCGAGTTC | |
| | AAGTTCCTGGAGAACGGCTGGGTGGAGATCACCGCCAACA | |
| | GCAGCCGCCTGGTGCACCTGAACATGCCCGAGAGCGAGA | |
| | ACTACCGCCGCGTGGTGGTGAACAACATGGACAAGACCGC | |
| | CGTGAACGGCAACATGGCCCTGGACGACATCCACGCCCAG | |
| | ATCGTGACCCCCTGGAGCCTGGTGGACGCCAACGCCTGGG | |
| | GCGTGTGGTTCAACCCCGGCGACTGGCAGCTGATCGTGAA | |
| | CACCATGAGCGAGCTGCACCTGGTGAGCTTCGAGCAGGAG | |
| | ATCTTCAACGTGGTGCTGAAGACCGTGAGCGAGAGCGCCA | |
| | CCCAGCCCCCCACCAAGGTGTACAACAACGACCTGACCGC | |
| | CAGCCTGATGGTGGCCCTGGACAGCAACAACACCATGCCC | |
| | TTCACCCCCGCCGCCATGCGCAGCGAGACCCTGGGCTTCT | |
| | ACCCCTGGAAGCCCACCATCCCCACCCCCTGGCGCTACTAC | |
| | TTCCAGTGGGACCGCACCCTGATCCCCAGCCACACCGGCA | |
| | CCAGCGGCACCCCCACCAACATCTACCACGGCACCGACCC | |
| | CGACGACGTGCAGTTCTACACCATCGAGAACAGCGTGCCC | |
| | GTGCACCTGCTGCGCACCGGCGACGAGTTCGCCACCGGCA | |
| | CCTTCTTCTTCGACTGCAAGCCCTGCCGCCTGACCCACACC | |
| | TGGCAGACCAACCGCGCCCTGGGCCTGCCCCCCTTCCTGA | |
| | ACAGCCTGCCCCAGAGCGAGGGCGCCACCAACTTCGGCG | |
| | ACATCGGCGTGCAGCAGGACAAGCGCCGCGGCGTGACCC | |
| | AGATGGGCAACACCAACTACATCACCGAGGCCACCATCAT | |
| | GCGCCCCGCCGAGGTGGGCTACAGCGCCCCCTACTACAGC | |
| | TTCGAGGCCAGCACCCAGGGCCCCTTCAAGACCCCCATCG | |
| | CCGCCGGCCGCGGCGGCGCCCAGACCTACGAGAACCAGG | |
| | CCGCCGACGGCGACCCCCGCTACGCCTTCGGCCGCCAGCA | |
| | CGGCCAGAAGACCACCACCACCGGCGAGACCCCCGAGCG | |
| | CTTCACCTACATCGCCCACCAGGACACCGGCCGCTACCCC | |
| | GAGGGCGACTGGATCCAGAACATCAACTTCAACCTGCCCG | |
| | TGACCAACGACAACGTGCTGCTGCCCACCGACCCCATCGG | |
| | CGGCAAGACCGGCATCAACTACACCAACATCTTCAACACC | |
| | TACGGCCCCCTGACCGCCCTGAACAACGTGCCCCCCGTGT | |
| | ACCCCAACGGCCAGATCTGGGACAAGGAGTTCGACACCG | |
| | ACCTGAAGCCCCGCCTGCACGTGAACGCCCCCTTCGTGTG | |
| | CCAGAACAACTGCCCCGGCCAGCTGTTCGTGAAGGTGGCC | |
| | CCCAACCTGACCAACGAGTACGACCCCGACGCCAGCGCCA | |
| | ACATGAGCCGCATCGTGACCTACAGCGACTTCTGGTGGAA | |
| | GGGCAAGCTGGTGTTCAAGGCCAAGCTGCGCGCCAGCCA | |
| | CACCTGGAACCCCATCCAGCAGATGAGCATCAACGTGGAC | |
| | AACCAGTTCAACTACGTGCCCAGCAACATCGGCGGCATGA | |
| | AGATCGTGTACGAGAAGAGCCAGCTGGCCCCCCGCAAGCT | |
| | GTACTAATAACTCGAGCATGCATCTAGAGGTACATCTAGATA | |
| | GAGCTCGCTGATCAGCCTCGACTGTGCCTTCTAGTTGCCAG | |
| | CCATCTGTTGTTTGCCCCTCCCCCGTGCCTTCCTTGACCCT | |
| | GGAAGGTGCCACTCCCACTGTCCTTTCCTAATAAAATGAGG | |
| | AAATTGCATCGCATTGTCTGAGTAGGTGTCATTCTATTCTGG | |
| | GGGGGGGGTGGGGCAGGACAGCAAGGGGGAGGATTGGG | |
| | AAGACAATAGCAGGCATGCTGGGGA | |
| Exemplary | ATCATGGAGATAATTAAAATGATAACCATCTCGCAAATAAAT | SEQ ID |
| CuV | AAGTATTTTACTGTTTTCGTAACAGTTTTGTAATAAAAAAAC | NO: 131 |
| Construct 1 | CTATAAATATTCCCTCGACGAAGACTTGATCACCCGGGGGA | |
| comprising | TCCCCTGTTAAGCTGGCTCCAGCTATTAGAAAAGCCAGAG | |
| a proto- | GTTACAACTTCCTAGGACCCTTCAATCAAGACTTCAACAAA | |
| parvovirus | GAACCAACTAATCCATCAGACAACGCTGCAAAACAACACG | |
| variant | ATTTGGAATACAACAAACTAATCAACCAAGGACACAATCCT | |
| VP1 capsid | TATTGGTACTACAACAAAGCTGACGAAGACTTCATCAAAG | |
| coding | CAACAGATCAAGCACCAGACTGGGGAGGAAAATTTGGCA | |
| sequence | ACTTCATCTTCAGAGCCAAAAAACACATCGCTCCAGAACT | |
| Ph-v5UTR- | GGCACCACCAGCAAAAAAGAAAAGCAAAACCAAACACAG | |
| CuV- | TGAACCAGAATTCAGCCACAAACACATCAAACCAGGCACC | |
| CTG_GTC- | AAAAGAGGTAAGCCTTTTCATATTTTTGTAAACCTTGCTAG | |
| Del- | AAAAAGAGCCCGCATGTCAGAACCAGCTAATGATACAAAT | |
| WVPPG | GAACAACCAGACAACTCCCCTGTTGAACAGGGTGCTGGTC | |
| | AAATTGGAGGAGGTGGAGGTGGAGGTGGAAGCGGTGTCG | |
| | GGCACAGCACTGGTGATTATAATAATAGGACTGAGTTTATTT | |
| | ATCATGGTGATGAAGTCACAATTATTTGCCACTCTACAAGA | |
| | CTGGTTCACATCAATATGTCAGACAGGGAAGACTACATCAT | |
| | CTATGAAACAGACAGAGGACCACTCTTTCCTACCACTCAG | |
| | GACCTGCAGGGTAGAGACACTCTAAATGACTCTTACCATGC | |
| | CAAAGTAGAAACACCATGGAAACTACTCCATGCAAACAGC | |
| | TGGGGCTGCTGGTTTTCACCAGCAGACTTCCAACAAATGA | |
| | TCACCACATGCAGAGACATAGCACCAATAAAAATGCACCA | |
| | AAAAATAGAAAACATTGTCATCAAAACAGTCAGTAAAACA | |
| | GGCACAGGAGAAACAGAAACAACCAACTACAACAATGAC | |
| | CTCACAGCACTCCTACAAATTGCACAAGACAACAGTAACC | |
| | TACTACCATGGGCTGCAGATAACTTTTATATAGACTCAGTAG | |
| | GTTACGTTCCATGGAGAGCATGCAAACTACCAACCTACTGC | |
| | TACCACGTAGACACTTGGAATACAATTGACATAAACCAAGC | |
| | AGACACACCAAACCAATGGAGAGAAATCAAAAAAGGCAT | |
| | CCAATGGGACAATATCCAATTCACACCACTAGAAACTATGA | |
| | TAAACATTGACTTACTAAGAACAGGAGATGCCTGGGAATCT | |
| | GGTAACTACAATTTCCACACAAAACCAACAAACCTAGCTT | |
| | ACCATTGGCAATCACAAAGACACACAGGCAGCTGTCACCC | |
| | AACAGTAGCACCTCTAGTTGAAAGAGGACAAGGAACCAA | |
| | CATACAATCAGTAAACTGTTGGCAATGGGGAGACAGAAAC | |
| | AATCCAAGCTCTGCATCAACCAGAGTATCCAATATACATATT | |
| | GGATACTCATTTCCAGAATGGCAAATCCACTACTCAACAGG | |
| | AGGACCAGTAATTAATCCAGGCAGTGCATTCTCACAAGCA | |
| | CCATGGGGCTCAACAACTGAAGGCACCAGACTAACCCAAG | |
| | GTGCATCTGAAAAAGCCATCTATGACTGGTCCCATGGAGAT | |
| | GACCAACCAGGAGCCAGAGAAACCTGGTGGCAAAACAAC | |
| | CAACATGTAACAGGACAAACTGACTGGGCACCAAAAAATG | |
| | CACACACCTCAGAACTCAACAACAATGTACCAGCAGCCAC | |
| | ACACTTCTGGAAAAACAGCTATCACAACACCTTCTCACCAT | |
| | TCACTGCAGTAGATGATCATGGACCACAATATCCATGGGGA | |
| | GCCATCTGGGGAAAATACCCAGACACCACACACAAACCAA | |
| | TGATGTCAGCTCACGCACCATTCCTACTTCATGGACCACCT | |
| | GGACAACTCTTTGTAAAACTAGCACCAAACTATACAGACA | |
| | CACTTGACAACGGAGGTGTAACACATCCCAGAATCGTCAC | |
| | ATATGGAACCTTCTGGTGGTCAGGACAACTCATCTTTAAAG | |
| | GAAAACTACGCACTCCAAGACAATGGAATACCTACAACCT | |
| | ACCAAGCCTAGACAAAAGAGAAACCATGAAAAACACAGT | |
| | ACCAAATGAAGTTGGTCACTTTGAACTACCATACATGCCAG | |
| | GAAGATGTCTACCAAACTACACATTGTAACTCGAGGCATGC | |
| | GGTACCAAGCTTGTCGAGAAGTACTAGAGGATCATAATCAG | |
| | CCATACCACATTTGTAGAGGTTTTACTTGCTTTAAAAAACC | |
| | TCCCACACCTCCCCCTGAACCTGAAACATAAAATGAATGCA | |
| | ATTGTTGTTGTTAACTTGTTTATTGCAGCTTATAATGGTTAC | |
| | AAATAAAGCAATAGCATCACAAATTTCACAAATAAAGCATT | |
| | TTTTTCACTGCATTCTAGTTGTGGTTTGTCCAAACTCATCAA | |
| | TGTATCTTATCATGTCTGGATC | |
| Exemplary | ATCATGGAGATAATTAAAATGATAACCATCTCGCAAATAAAT | SEQ ID |
| CuV | AAGTATTTTACTGTTTTCGTAACAGTTTTGTAATAAAAAAAC | NO: 132 |
| Construct 2 | CTATAAATACTCCGGACTACTGATACCGTCCCACTTTCGGG | |
| comprising | CGCTTACCTGCCGCCACGCCAGCTATTAGAAAAGCCAGAG | |
| a proto- | GATACAACTTCCTAGGACCCTTCAATCAAGACTTCAACAAA | |
| parvovirus | GAACCAACTAATCCATCAGACAACGCTGCAAAACAACACG | |
| variant | ATTTGGAATACAACAAACTAATCAACCAAGGACACAATCCT | |
| VP1 capsid | TATTGGTACTACAACAAAGCTGACGAAGACTTCATCAAAG | |
| coding | CAACAGATCAAGCACCAGACTGGGGAGGAAAATTTGGCA | |
| sequence | ACTTCATCTTCAGAGCCAAAAAACACATCGCTCCAGAACT | |
| Ph-Kozak- | GGCACCACCAGCAAAAAAGAAAAGCAAAACCAAACACAG | |
| CuV-ACG- | TGAACCAGAATTCAGCCACAAACACATCAAACCAGGCACC | |
| Del- | AAAAGAGGTAAGCCTTTTCATATTTTTGTAAACCTTGCTAG | |
| WVPPG | AAAAAGAGCCCGCATGTCAGAACCAGCTAATGATACAAAT | |
| | GAACAACCAGACAACTCCCCTGTTGAACAGGGTGCTGGTC | |
| | AAATTGGAGGAGGTGGAGGTGGAGGTGGAAGCGGTGTCG | |
| | GGCACAGCACTGGTGATTATAATAATAGGACTGAGTTTATTT | |
| | ATCATGGTGATGAAGTCACAATTATTTGCCACTCTACAAGA | |
| | CTGGTTCACATCAATATGTCAGACAGGGAAGACTACATCAT | |
| | CTATGAAACAGACAGAGGACCACTCTTTCCTACCACTCAG | |
| | GACCTGCAGGGTAGAGACACTCTAAATGACTCTTACCATGC | |
| | CAAAGTAGAAACACCATGGAAACTACTCCATGCAAACAGC | |
| | TGGGGCTGCTGGTTTTCACCAGCAGACTTCCAACAAATGA | |
| | TCACCACATGCAGAGACATAGCACCAATAAAAATGCACCA | |
| | AAAAATAGAAAACATTGTCATCAAAACAGTCAGTAAAACA | |
| | GGCACAGGAGAAACAGAAACAACCAACTACAACAATGAC | |
| | CTCACAGCACTCCTACAAATTGCACAAGACAACAGTAACC | |
| | TACTACCATGGGCTGCAGATAACTTTTATATAGACTCAGTAG | |
| | GTTACGTTCCATGGAGAGCATGCAAACTACCAACCTACTGC | |
| | TACCACGTAGACACTTGGAATACAATTGACATAAACCAAGC | |
| | AGACACACCAAACCAATGGAGAGAAATCAAAAAAGGCAT | |
| | CCAATGGGACAATATCCAATTCACACCACTAGAAACTATGA | |
| | TAAACATTGACTTACTAAGAACAGGAGATGCCTGGGAATCT | |
| | GGTAACTACAATTTCCACACAAAACCAACAAACCTAGCTT | |
| | ACCATTGGCAATCACAAAGACACACAGGCAGCTGTCACCC | |
| | AACAGTAGCACCTCTAGTTGAAAGAGGACAAGGAACCAA | |
| | CATACAATCAGTAAACTGTTGGCAATGGGGAGACAGAAAC | |
| | AATCCAAGCTCTGCATCAACCAGAGTATCCAATATACATATT | |
| | GGATACTCATTTCCAGAATGGCAAATCCACTACTCAACAGG | |
| | AGGACCAGTAATTAATCCAGGCAGTGCATTCTCACAAGCA | |
| | CCATGGGGCTCAACAACTGAAGGCACCAGACTAACCCAAG | |
| | GTGCATCTGAAAAAGCCATCTATGACTGGTCCCATGGAGAT | |
| | GACCAACCAGGAGCCAGAGAAACCTGGTGGCAAAACAAC | |
| | CAACATGTAACAGGACAAACTGACTGGGCACCAAAAAATG | |
| | CACACACCTCAGAACTCAACAACAATGTACCAGCAGCCAC | |
| | ACACTTCTGGAAAAACAGCTATCACAACACCTTCTCACCAT | |
| | TCACTGCAGTAGATGATCATGGACCACAATATCCATGGGGA | |
| | GCCATCTGGGGAAAATACCCAGACACCACACACAAACCAA | |
| | TGATGTCAGCTCACGCACCATTCCTACTTCATGGACCACCT | |
| | GGACAACTCTTTGTAAAACTAGCACCAAACTATACAGACA | |
| | CACTTGACAACGGAGGTGTAACACATCCCAGAATCGTCAC | |
| | ATATGGAACCTTCTGGTGGTCAGGACAACTCATCTTTAAAG | |
| | GAAAACTACGCACTCCAAGACAATGGAATACCTACAACCT | |
| | ACCAAGCCTAGACAAAAGAGAAACCATGAAAAACACAGT | |
| | ACCAAATGAAGTTGGTCACTTTGAACTACCATACATGCCAG | |
| | GAAGATGTCTACCAAACTACACATTGTAACTCGAGGCATGC | |
| | GGTACCAAGCTTGTCGAGAAGTACTAGAGGATCATAATCAG | |
| | CCATACCACATTTGTAGAGGTTTTACTTGCTTTAAAAAACC | |
| | TCCCACACCTCCCCCTGAACCTGAAACATAAAATGAATGCA | |
| | ATTGTTGTTGTTAACTTGTTTATTGCAGCTTATAATGGTTAC | |
| | AAATAAAGCAATAGCATCACAAATTTCACAAATAAAGCATT | |
| | TTTTTCACTGCATTCTAGTTGTGGTTTGTCCAAACTCATCAA | |
| | TGTATCTTATCATGTCTGGATC | |
| Exemplary | GACATTGATTATTGACTAGTTATTAATAGTAATCAATTACGG | SEQ ID |
| CuV | GGTCATTAGTTCATAGCCCATATATGGAGTTCCGCGTTACAT | NO: 133 |
| Construct 3 | AACTTACGGTAAATGGCCCGCCTGGCTGACCGCCCAACGA | |
| comprising | CCCCCGCCCATTGACGTCAATAATGACGTATGTTCCCATAGT | |
| a variant | AACGCCAATAGGGACTTTCCATTGACGTCAATGGGTGGACT | |
| VP1 capsid | ATTTACGGTAAACTGCCCACTTGGCAGTACATCAAGTGTAT | |
| coding | CATATGCCAAGTACGCCCCCTATTGACGTCAATGACGGTAA | |
| sequence | ATGGCCCGCCTGGCATTATGCCCAGTACATGACCTTATGGG | |
| CMV- | ACTTTCCTACTTGGCAGTACATCTACGTATTAGTCATCGCTA | |
| codopt_CuV_ | TTACCATGGTGATGCGGTTTTGGCAGTACATCAATGGGCGT | |
| VP1_delta_ | GGATAGCGGTTTGACTCACGGGGATTTCCAAGTCTCCACCC | |
| WVPPG | CATTGACGTCAATGGGAGTTTGTTTTGGCACCAAAATCAAC | |
| | GGGACTTTCCAAAATGTCGTAACAACTCCGCCCCATTGACG | |
| | CAAATGGGCGGTAGGCGTGTACGGTGGGAGGTCTATATAAG | |
| | CAGAGCTCTCTGGCTAACTAGAGAACCCACTGCTTACTGG | |
| | CTTATCGAAATTAATACGACTCACTATAGGGAGACCCAAGC | |
| | TTGGTACCGGACTCTAGAGGATCCGGTACTCGAGGAACTG | |
| | AAAAACCAGAAAGTTAACTGGTAAGTTTAGTCTTTTTGTCT | |
| | TTTATTTCAGGTCCCGGATCCGGTGGTGGTGCAAATCAAAG | |
| | AACTGCTCCTCAGTGGATGTTGCCTTTACTTCTAGGCCTGT | |
| | ACGGAAGTGTTACTTCTGCTCTAAAAGCTGCGGAATTGTAC | |
| | CCGCGGAAGCTTCCTAGGCCGCCACCATGCCCGCCATCCG | |
| | CAAGGCCCGCGGCTACAACTTCCTGGGCCCCTTCAACCAG | |
| | GACTTCAACAAGGAGCCCACCAACCCCAGCGACAACGCC | |
| | GCCAAGCAGCACGACCTGGAGTACAACAAGCTGATCAACC | |
| | AGGGCCACAACCCCTACTGGTACTACAACAAGGCCGACGA | |
| | GGACTTCATCAAGGCCACCGACCAGGCCCCCGACTGGGGC | |
| | GGCAAGTTCGGCAACTTCATCTTCCGCGCCAAGAAGCACA | |
| | TCGCCCCCGAGCTGGCCCCCCCCGCCAAGAAGAAGAGCA | |
| | AGACCAAGCACAGCGAGCCCGAGTTCAGCCACAAGCACA | |
| | TCAAGCCCGGCACCAAGCGCGGCAAGCCCTTCCACATCTT | |
| | CGTGAACCTGGCCCGCAAGCGCGCCCGCATGAGCGAGCCC | |
| | GCCAACGACACCAACGAGCAGCCCGACAACAGCCCCGTG | |
| | GAGCAGGGCGCCGGCCAGATCGGCGGCGGCGGCGGCGGC | |
| | GGCGGCAGCGGCGTGGGCCACAGCACCGGCGACTACAAC | |
| | AACCGCACCGAGTTCATCTACCACGGCGACGAGGTGACCA | |
| | TCATCTGCCACAGCACCCGCCTGGTGCACATCAACATGAG | |
| | CGACCGCGAGGACTACATCATCTACGAGACCGACCGCGGC | |
| | CCCCTGTTCCCCACCACCCAGGACCTGCAGGGCCGCGACA | |
| | CCCTGAACGACAGCTACCACGCCAAGGTGGAGACCCCCTG | |
| | GAAGCTGCTGCACGCCAACAGCTGGGGCTGCTGGTTCAGC | |
| | CCCGCCGACTTCCAGCAGATGATCACCACCTGCCGCGACA | |
| | TCGCCCCCATCAAGATGCACCAGAAGATCGAGAACATCGT | |
| | GATCAAGACCGTGAGCAAGACCGGCACCGGCGAGACCGA | |
| | GACCACCAACTACAACAACGACCTGACCGCCCTGCTGCAG | |
| | ATCGCCCAGGACAACAGCAACCTGCTGCCCTGGGCCGCCG | |
| | ACAACTTCTACATCGACAGCGTGGGCTACGTGCCCTGGCG | |
| | CGCCTGCAAGCTGCCCACCTACTGCTACCACGTGGACACC | |
| | TGGAACACCATCGACATCAACCAGGCCGACACCCCCAACC | |
| | AGTGGCGCGAGATCAAGAAGGGCATCCAGTGGGACAACAT | |
| | CCAGTTCACCCCCCTGGAGACCATGATCAACATCGACCTGC | |
| | TGCGCACCGGCGACGCCTGGGAGAGCGGCAACTACAACTT | |
| | CCACACCAAGCCCACCAACCTGGCCTACCACTGGCAGAGC | |
| | CAGCGCCACACCGGCAGCTGCCACCCCACCGTGGCCCCCC | |
| | TGGTGGAGCGCGGCCAGGGCACCAACATCCAGAGCGTGA | |
| | ACTGCTGGCAGTGGGGCGACCGCAACAACCCCAGCAGCG | |
| | CCAGCACCCGCGTGAGCAACATCCACATCGGCTACAGCTT | |
| | CCCCGAGTGGCAGATCCACTACAGCACCGGCGGCCCCGTG | |
| | ATCAACCCCGGCAGCGCCTTCAGCCAGGCCCCCTGGGGCA | |
| | GCACCACCGAGGGCACCCGCCTGACCCAGGGCGCCAGCG | |
| | AGAAGGCCATCTACGACTGGAGCCACGGCGACGACCAGCC | |
| | CGGCGCCCGCGAGACCTGGTGGCAGAACAACCAGCACGT | |
| | GACCGGCCAGACCGACTGGGCCCCCAAGAACGCCCACAC | |
| | CAGCGAGCTGAACAACAACGTGCCCGCCGCCACCCACTTC | |
| | TGGAAGAACAGCTACCACAACACCTTCAGCCCCTTCACCG | |
| | CCGTGGACGACCACGGCCCCCAGTACCCCTGGGGCGCCAT | |
| | CTGGGGCAAGTACCCCGACACCACCCACAAGCCCATGATG | |
| | AGCGCCCACGCCCCCTTCCTGCTGCACGGCCCCCCCGGCC | |
| | AGCTGTTCGTGAAGCTGGCCCCCAACTACACCGACACCCT | |
| | GGACAACGGCGGCGTGACCCACCCCCGCATCGTGACCTAC | |
| | GGCACCTTCTGGTGGAGCGGCCAGCTGATCTTCAAGGGCA | |
| | AGCTGCGCACCCCCCGCCAGTGGAACACCTACAACCTGCC | |
| | CAGCCTGGACAAGCGCGAGACCATGAAGAACACCGTGCC | |
| | CAACGAGGTGGGCCACTTCGAGCTGCCCTACATGCCCGGC | |
| | CGCTGCCTGCCCAACTACACCCTGTAATAACTCGAGCATGC | |
| | ATCTAGAGGTACATCTAGATAGAGCTCGCTGATCAGCCTCG | |
| | ACTGTGCCTTCTAGTTGCCAGCCATCTGTTGTTTGCCCCTC | |
| | CCCCGTGCCTTCCTTGACCCTGGAAGGTGCCACTCCCACT | |
| | GTCCTTTCCTAATAAAATGAGGAAATTGCATCGCATTGTCT | |
| | GAGTAGGTGTCATTCTATTCTGGGGGGTGGGGTGGGGCAG | |
| | GACAGCAAGGGGGAGGATTGGGAAGACAATAGCAGGCAT | |
| | GCTGGGGA | |
| Exemplary | ATCATGGAGATAATTAAAATGATAACCATCTCGCAAATAAAT | SEQ ID |
| CuV | AAGTATTTTACTGTTTTCGTAACAGTTTTGTAATAAAAAAAC | NO: 134 |
| Construct 4 | CTATAAATACTCCGGACTACTGATACCGTCCCACTTTCGGG | |
| comprising | CGCTTACCTGCCGCCACGCCAGCTATTAGAAAAGCCAGAG | |
| a proto- | GACCCTTCAATCAAGACTTCAACAAAGAACCAACTAATCC | |
| parvovirus | ATCAGACAACGCTGCAAAACAACACGATTTGGAATACAAC | |
| variant | AAACTAATCAACCAAGGACACAATCCTTATTGGTACTACAA | |
| VP1 capsid | CAAAGCTGACGAAGACTTCATCAAAGCAACAGATCAAGCA | |
| coding | CCAGACTGGGGAGGAAAATTTGGCAACTTCATCTTCAGAG | |
| sequence | CCAAAAAACACATCGCTCCAGAACTGGCACCACCAGCAA | |
| Ph-Kozak- | AAAAGAAAAGCAAAACCAAACACAGTGAACCAGAATTCA | |
| CuV-ACG- | GCCACAAACACATCAAACCAGGCACCAAAAGAGGTAAGC | |
| Del- | CTTTTCATATTTTTGTAAACCTTGCTAGAAAAAGAGCCCGC | |
| WVPPGYN | ATGTCAGAACCAGCTAATGATACAAATGAACAACCAGACA | |
| FLG | ACTCCCCTGTTGAACAGGGTGCTGGTCAAATTGGAGGAGG | |
| | TGGAGGTGGAGGTGGAAGCGGTGTCGGGCACAGCACTGG | |
| | TGATTATAATAATAGGACTGAGTTTATTTATCATGGTGATGA | |
| | AGTCACAATTATTTGCCACTCTACAAGACTGGTTCACATCA | |
| | ATATGTCAGACAGGGAAGACTACATCATCTATGAAACAGAC | |
| | AGAGGACCACTCTTTCCTACCACTCAGGACCTGCAGGGTA | |
| | GAGACACTCTAAATGACTCTTACCATGCCAAAGTAGAAAC | |
| | ACCATGGAAACTACTCCATGCAAACAGCTGGGGCTGCTGG | |
| | TTTTCACCAGCAGACTTCCAACAAATGATCACCACATGCAG | |
| | AGACATAGCACCAATAAAAATGCACCAAAAAATAGAAAAC | |
| | ATTGTCATCAAAACAGTCAGTAAAACAGGCACAGGAGAAA | |
| | CAGAAACAACCAACTACAACAATGACCTCACAGCACTCCT | |
| | ACAAATTGCACAAGACAACAGTAACCTACTACCATGGGCT | |
| | GCAGATAACTTTTATATAGACTCAGTAGGTTACGTTCCATGG | |
| | AGAGCATGCAAACTACCAACCTACTGCTACCACGTAGACA | |
| | CTTGGAATACAATTGACATAAACCAAGCAGACACACCAAA | |
| | CCAATGGAGAGAAATCAAAAAAGGCATCCAATGGGACAAT | |
| | ATCCAATTCACACCACTAGAAACTATGATAAACATTGACTT | |
| | ACTAAGAACAGGAGATGCCTGGGAATCTGGTAACTACAAT | |
| | TTCCACACAAAACCAACAAACCTAGCTTACCATTGGCAATC | |
| | ACAAAGACACACAGGCAGCTGTCACCCAACAGTAGCACCT | |
| | CTAGTTGAAAGAGGACAAGGAACCAACATACAATCAGTAA | |
| | ACTGTTGGCAATGGGGAGACAGAAACAATCCAAGCTCTGC | |
| | ATCAACCAGAGTATCCAATATACATATTGGATACTCATTTCC | |
| | AGAATGGCAAATCCACTACTCAACAGGAGGACCAGTAATT | |
| | AATCCAGGCAGTGCATTCTCACAAGCACCATGGGGCTCAA | |
| | CAACTGAAGGCACCAGACTAACCCAAGGTGCATCTGAAAA | |
| | AGCCATCTATGACTGGTCCCATGGAGATGACCAACCAGGA | |
| | GCCAGAGAAACCTGGTGGCAAAACAACCAACATGTAACA | |
| | GGACAAACTGACTGGGCACCAAAAAATGCACACACCTCA | |
| | GAACTCAACAACAATGTACCAGCAGCCACACACTTCTGGA | |
| | AAAACAGCTATCACAACACCTTCTCACCATTCACTGCAGTA | |
| | GATGATCATGGACCACAATATCCATGGGGAGCCATCTGGGG | |
| | AAAATACCCAGACACCACACACAAACCAATGATGTCAGCT | |
| | CACGCACCATTCCTACTTCATGGACCACCTGGACAACTCTT | |
| | TGTAAAACTAGCACCAAACTATACAGACACACTTGACAAC | |
| | GGAGGTGTAACACATCCCAGAATCGTCACATATGGAACCTT | |
| | CTGGTGGTCAGGACAACTCATCTTTAAAGGAAAACTACGC | |
| | ACTCCAAGACAATGGAATACCTACAACCTACCAAGCCTAG | |
| | ACAAAAGAGAAACCATGAAAAACACAGTACCAAATGAAG | |
| | TTGGTCACTTTGAACTACCATACATGCCAGGAAGATGTCTA | |
| | CCAAACTACACATTGTAACTCGAGGCATGCGGTACCAAGCT | |
| | TGTCGAGAAGTACTAGAGGATCATAATCAGCCATACCACAT | |
| | TTGTAGAGGTTTTACTTGCTTTAAAAAACCTCCCACACCTC | |
| | CCCCTGAACCTGAAACATAAAATGAATGCAATTGTTGTTGT | |
| | TAACTTGTTTATTGCAGCTTATAATGGTTACAAATAAAGCAA | |
| | TAGCATCACAAATTTCACAAATAAAGCATTTTTTTCACTGC | |
| | ATTCTAGTTGTGGTTTGTCCAAACTCATCAATGTATCTTATC | |
| | ATGTCTGGATC | |
| Exemplary | GACATTGATTATTGACTAGTTATTAATAGTAATCAATTACGG | SEQ ID |
| CuV | GGTCATTAGTTCATAGCCCATATATGGAGTTCCGCGTTACAT | NO: 135 |
| Construct 5 | AACTTACGGTAAATGGCCCGCCTGGCTGACCGCCCAACGA | |
| comprising | CCCCCGCCCATTGACGTCAATAATGACGTATGTTCCCATAGT | |
| a proto- | AACGCCAATAGGGACTTTCCATTGACGTCAATGGGTGGACT | |
| parvovirus | ATTTACGGTAAACTGCCCACTTGGCAGTACATCAAGTGTAT | |
| variant | CATATGCCAAGTACGCCCCCTATTGACGTCAATGACGGTAA | |
| VP1 capsid | ATGGCCCGCCTGGCATTATGCCCAGTACATGACCTTATGGG | |
| coding | ACTTTCCTACTTGGCAGTACATCTACGTATTAGTCATCGCTA | |
| sequence | TTACCATGGTGATGCGGTTTTGGCAGTACATCAATGGGCGT | |
| CMV- | GGATAGCGGTTTGACTCACGGGGATTTCCAAGTCTCCACCC | |
| codopt_CuV_ | CATTGACGTCAATGGGAGTTTGTTTTGGCACCAAAATCAAC | |
| VP1_delta_ | GGGACTTTCCAAAATGTCGTAACAACTCCGCCCCATTGACG | |
| WVPPG | CAAATGGGCGGTAGGCGTGTACGGTGGGAGGTCTATATAAG | |
| YNFLG | CAGAGCTCTCTGGCTAACTAGAGAACCCACTGCTTACTGG | |
| | CTTATCGAAATTAATACGACTCACTATAGGGAGACCCAAGC | |
| | TTGGTACCGGACTCTAGAGGATCCGGTACTCGAGGAACTG | |
| | AAAAACCAGAAAGTTAACTGGTAAGTTTAGTCTTTTTGTCT | |
| | TTTATTTCAGGTCCCGGATCCGGTGGTGGTGCAAATCAAAG | |
| | AACTGCTCCTCAGTGGATGTTGCCTTTACTTCTAGGCCTGT | |
| | ACGGAAGTGTTACTTCTGCTCTAAAAGCTGCGGAATTGTAC | |
| | CCGCGGAAGCTTCCTAGGCCGCCACCATGCCCGCCATCCG | |
| | CAAGGCCCGCGGCCCCTTCAACCAGGACTTCAACAAGGA | |
| | GCCCACCAACCCCAGCGACAACGCCGCCAAGCAGCACGA | |
| | CCTGGAGTACAACAAGCTGATCAACCAGGGCCACAACCCC | |
| | TACTGGTACTACAACAAGGCCGACGAGGACTTCATCAAGG | |
| | CCACCGACCAGGCCCCCGACTGGGGCGGCAAGTTCGGCA | |
| | ACTTCATCTTCCGCGCCAAGAAGCACATCGCCCCCGAGCT | |
| | GGCCCCCCCCGCCAAGAAGAAGAGCAAGACCAAGCACAG | |
| | CGAGCCCGAGTTCAGCCACAAGCACATCAAGCCCGGCACC | |
| | AAGCGCGGCAAGCCCTTCCACATCTTCGTGAACCTGGCCC | |
| | GCAAGCGCGCCCGCATGAGCGAGCCCGCCAACGACACCA | |
| | ACGAGCAGCCCGACAACAGCCCCGTGGAGCAGGGCGCCG | |
| | GCCAGATCGGCGGCGGCGGCGGCGGCGGCGGCAGCGGCG | |
| | TGGGCCACAGCACCGGCGACTACAACAACCGCACCGAGTT | |
| | CATCTACCACGGCGACGAGGTGACCATCATCTGCCACAGC | |
| | ACCCGCCTGGTGCACATCAACATGAGCGACCGCGAGGACT | |
| | ACATCATCTACGAGACCGACCGCGGCCCCCTGTTCCCCACC | |
| | ACCCAGGACCTGCAGGGCCGCGACACCCTGAACGACAGC | |
| | TACCACGCCAAGGTGGAGACCCCCTGGAAGCTGCTGCACG | |
| | CCAACAGCTGGGGCTGCTGGTTCAGCCCCGCCGACTTCCA | |
| | GCAGATGATCACCACCTGCCGCGACATCGCCCCCATCAAG | |
| | ATGCACCAGAAGATCGAGAACATCGTGATCAAGACCGTGA | |
| | GCAAGACCGGCACCGGCGAGACCGAGACCACCAACTACA | |
| | ACAACGACCTGACCGCCCTGCTGCAGATCGCCCAGGACAA | |
| | CAGCAACCTGCTGCCCTGGGCCGCCGACAACTTCTACATC | |
| | GACAGCGTGGGCTACGTGCCCTGGCGCGCCTGCAAGCTGC | |
| | CCACCTACTGCTACCACGTGGACACCTGGAACACCATCGA | |
| | CATCAACCAGGCCGACACCCCCAACCAGTGGCGCGAGATC | |
| | AAGAAGGGCATCCAGTGGGACAACATCCAGTTCACCCCCC | |
| | TGGAGACCATGATCAACATCGACCTGCTGCGCACCGGCGA | |
| | CGCCTGGGAGAGCGGCAACTACAACTTCCACACCAAGCCC | |
| | ACCAACCTGGCCTACCACTGGCAGAGCCAGCGCCACACCG | |
| | GCAGCTGCCACCCCACCGTGGCCCCCCTGGTGGAGCGCGG | |
| | CCAGGGCACCAACATCCAGAGCGTGAACTGCTGGCAGTGG | |
| | GGCGACCGCAACAACCCCAGCAGCGCCAGCACCCGCGTG | |
| | AGCAACATCCACATCGGCTACAGCTTCCCCGAGTGGCAGA | |
| | TCCACTACAGCACCGGCGGCCCCGTGATCAACCCCGGCAG | |
| | CGCCTTCAGCCAGGCCCCCTGGGGCAGCACCACCGAGGGC | |
| | ACCCGCCTGACCCAGGGCGCCAGCGAGAAGGCCATCTACG | |
| | ACTGGAGCCACGGCGACGACCAGCCCGGCGCCCGCGAGA | |
| | CCTGGTGGCAGAACAACCAGCACGTGACCGGCCAGACCG | |
| | ACTGGGCCCCCAAGAACGCCCACACCAGCGAGCTGAACA | |
| | ACAACGTGCCCGCCGCCACCCACTTCTGGAAGAACAGCTA | |
| | CCACAACACCTTCAGCCCCTTCACCGCCGTGGACGACCAC | |
| | GGCCCCCAGTACCCCTGGGGCGCCATCTGGGGCAAGTACC | |
| | CCGACACCACCCACAAGCCCATGATGAGCGCCCACGCCCC | |
| | CTTCCTGCTGCACGGCCCCCCCGGCCAGCTGTTCGTGAAG | |
| | CTGGCCCCCAACTACACCGACACCCTGGACAACGGCGGCG | |
| | TGACCCACCCCCGCATCGTGACCTACGGCACCTTCTGGTGG | |
| | AGCGGCCAGCTGATCTTCAAGGGCAAGCTGCGCACCCCCC | |
| | GCCAGTGGAACACCTACAACCTGCCCAGCCTGGACAAGCG | |
| | CGAGACCATGAAGAACACCGTGCCCAACGAGGTGGGCCA | |
| | CTTCGAGCTGCCCTACATGCCCGGCCGCTGCCTGCCCAACT | |
| | ACACCCTGTAATAACTCGAGCATGCATCTAGAGGTACATCT | |
| | AGATAGAGCTCGCTGATCAGCCTCGACTGTGCCTTCTAGTT | |
| | GCCAGCCATCTGTTGTTTGCCCCTCCCCCGTGCCTTCCTTG | |
| | ACCCTGGAAGGTGCCACTCCCACTGTCCTTTCCTAATAAAA | |
| | TGAGGAAATTGCATCGCATTGTCTGAGTAGGTGTCATTCTA | |
| | TTCTGGGGGGTGGGGTGGGGCAGGACAGCAAGGGGGAGG | |
| | ATTGGGAAGACAATAGCAGGCATGCTGGGGA | |
| Exemplary | CATGGAGATAATTAAAATGATAACCATCTCGCAAATAAATAA | SEQ ID |
| FPV | GTATTTTACTGTTTTCGTAACAGTTTTGTAATAAAAAAACCT | NO: 136 |
| Construct 1 | ATAAAATTCCGGATTATTCATACCGTCCCACCATCGGGCGCG | |
| comprising | GATCTCCTGTTAAGCTGGCACCTCCGGCAAAGAGAGCCAG | |
| a proto- | GAGAGGATATAAATATCTTGGGCCTGGGAACAGTCTTGACC | |
| parvovirus | AAGGAGAACCAACTAACCCTTCTGACGCCGCTGCAAAAGA | |
| variant | ACACGACGAAGCTTACGCTGCTTATCTTCGCTCTGGTAAAA | |
| VP1 capsid | ACCCATACTTATATTTCTCGCCAGCAGATCAACGCTTTATAG | |
| coding | ATCAAACTAAGGACGCTAAAGATTGGGGGGGGAAAATAGG | |
| sequence | ACATTATTTTTTTAGAGCTAAAAAGGCAATTGCTCCAGTATT | |
| Ph-v5UTR- | AACTGATACACCAGATCATCCATCAACATCAAGACCAACAA | |
| FPV-VP1- | AACCAACTAAAAGAAGTAAACCACCACCTCATATTTTCATC | |
| CTG-Del- | AATCTTGCAAAAAAAAAAAAAGCCGGTGCAGGACAAGTA | |
| LVPPG | AAAAGAGACAATCTTGCACCAATGAGTGATGGAGCAGTTC | |
| | AACCAGACGGTGGTCAACCTGCTGTCAGAAATGAAAGAG | |
| | CTACAGGATCTGGGAACGGGTCTGGAGGCGGGGGTGGTGG | |
| | TGGTTCTGGGGGTGTGGGGATTTCTACGGGTACTTTCAATA | |
| | ATCAGACGGAATTTAAATTTTTGGAAAACGGATGGGTGGA | |
| | AATCACAGCAAACTCAAGCAGACTTGTACATTTAAATATGC | |
| | CAGAAAGTGAAAATTATAAAAGAGTAGTTGTAAATAATATG | |
| | GATAAAACTGCAGTTAAAGGAAACATGGCTTTAGATGATAT | |
| | TCATGTACAAATTGTAACACCTTGGTCATTGGTTGATGCAA | |
| | ATGCTTGGGGAGTTTGGTTTAATCCAGGAGATTGGCAACTA | |
| | ATTGTTAATACTATGAGTGAGTTGCATTTAGTTAGTTTTGAA | |
| | CAAGAAATTTTTAATGTTGTTTTAAAGACTGTTTCAGAATC | |
| | TGCTACTCAGCCACCAACTAAAGTTTATAATAATGATTTAAC | |
| | TGCATCATTGATGGTTGCATTAGATAGTAATAATACTATGCC | |
| | ATTTACTCCAGCAGCTATGAGATCTGAGACATTGGGTTTTTA | |
| | TCCATGGAAACCAACCATACCAACTCCATGGAGATATTATTT | |
| | TCAATGGGATAGAACATTAATACCATCTCATACTGGAACTAG | |
| | TGGCACACCAACAAATATATACCATGGTACAGATCCAGATG | |
| | ATGTTCAATTTTATACTATTGAAAATTCTGTGCCAGTACACT | |
| | TACTAAGAACAGGTGATGAATTTGCTACAGGAACATTTTTT | |
| | TTTGATTGTAAACCATGTAGACTAACACATACATGGCAAAC | |
| | AAATAGAGCATTGGGCTTACCACCATTTTTAAATTCTTTGCC | |
| | TCAATCTGAAGGAGCTACTAACTTTGGTGATATAGGAGTTC | |
| | AACAAGATAAAAGACGTGGTGTAACTCAAATGGGAAATAC | |
| | AAACTATATTACTGAAGCTACTATTATGAGACCAGCTGAGG | |
| | TTGGTTATAGTGCACCATATTATTCTTTTGAGGCGTCTACAC | |
| | AAGGGCCATTTAAAACACCTATTGCAGCAGGACGGGGGGG | |
| | AGCGCAAACAGATGAAAATCAAGCAGCAGATGGTGATCCA | |
| | AGATATGCATTTGGTAGACAACATGGTCAAAAAACTACCAC | |
| | AACAGGAGAAACACCTGAGAGATTTACATATATAGCACATC | |
| | AAGATACAGGAAGATATCCAGAAGGAGATTGGATTCAAAA | |
| | TATTAACTTTAACCTTCCTGTAACAAATGATAATGTATTGCT | |
| | ACCAACAGATCCAATTGGAGGTAAAACAGGAATTAACTATA | |
| | CTAATATATTTAATACTTATGGTCCTTTAACTGCATTAAATAA | |
| | TGTACCACCAGTTTATCCAAATGGTCAAATTTGGGATAAAG | |
| | AATTTGATACTGACTTAAAACCAAGACTTCATGTAAATGCA | |
| | CCATTTGTTTGTCAAAATAATTGTCCTGGTCAATTATTTGTA | |
| | AAAGTTGCGCCTAATTTAACAAATGAATATGATCCTGATGC | |
| | ATCTGCTAATATGTCAAGAATTGTAACTTACTCAGATTTTTG | |
| | GTGGAAAGGTAAATTAGTATTTAAAGCTAAACTAAGAGCCT | |
| | CTCATACTTGGAATCCAATTCAACAAATGAGTATTAATGTAG | |
| | ATAACCAATTTAACTATGTACCAAGTAATATTGGAGCTATGA | |
| | AAATTGTATATGAAAAATCTCAACTAGCACCTAGAAAATTAT | |
| | ATTAACTCGAGGCATGCGGTACCAAGCTTGTCGAGAAGTA | |
| | CTAGAGGATCATAATCAGCCATACCACATTTGTAGAGGTTTT | |
| | ACTTGCTTTAAAAAACCTCCCACACCTCCCCCTGAACCTGA | |
| | AACATAAAATGAATGCAATTGTTGTTGTTAACTTGTTTATTG | |
| | CAGCTTATAATGGTTACAAATAAAGCAATAGCATCACAAAT | |
| | TTCACAAATAAAGCATTTTTTTCACTGCATTCTAGTTGTGGT | |
| | TTGTCCAAACTCATCAATGTATCTTATCATGTCTGGATC | |
| Exemplary | ATCATGGAGATAATTAAAATGATAACCATCTCGCAAATAAAT | SEQ ID |
| MVM | AAGTATTTTACTGTTTTCGTAACAGTTTTGTAATAAAAAAAC | NO: 137 |
| Construct 1 | CTATAAATACTCCGGACTACTGATACCGTCCCACTTTCGGG | |
| comprising | CGCTTACCTGCCGCCACGGCGCCTCCAGCTAAAAGAGCTA | |
| a proto- | AAAGAGGCTACAAGTACCTGGGACCAGGGAACAGCCTTG | |
| parvovirus | ACCAAGGAGAACCAACCAATCCATCTGACGCCGCTGCCAA | |
| variant | AGAGCACGACGAGGCCTACGATCAATACATCAAATCTGGA | |
| VP1 capsid | AAAAATCCTTACCTGTACTTCTCTGCTGCTGATCAACGCTT | |
| coding | TATTGACCAAACCAAGGACGCCAAAGACTGGGGAGGCAA | |
| sequence | GGTTGGTCACTACTTTTTTAGAACCAAGCGCGCTTTTGCAC | |
| Ph-Kozak- | CTAAGCTTGCTACTGACTCTGAACCTGGAACTTCTGGTGTA | |
| MVM-VP1- | AGCAGAGCTGGTAAACGCACTAGACCACCTGCTTACATTTT | |
| ACG-Del- | TATTAACCAAGCCAGAGCTAAAAAAAAACTTACTTCTTCTG | |
| WVPPG | CTGCACAGCAAAGCAGTCAAACCATGAGTGATGGCACCAG | |
| | CCAACCTGACAGCGGAAACGCTGTCCACTCAGCTGCAAGA | |
| | GTTGAACGAGCAGCTGACGGCCCTGGAGGCTCTGGGGGT | |
| | GGGGGCTCTGGCGGGGGTGGGGTTGGTGTTTCTACTGGGT | |
| | CTTATGATAATCAAACGCATTATAGATTCTTGGGTGACGGCT | |
| | GGGTAGAAATTACTGCACTAGCAACTAGACTAGTACATTTA | |
| | AACATGCCTAAATCAGAAAACTATTGCAGAATCAGAGTTCA | |
| | CAATACAACAGACACATCAGTCAAAGGCAACATGGCAAAA | |
| | GATGATGCTCATGAGCAAATTTGGACACCATGGAGCTTGGT | |
| | GGATGCTAATGCTTGGGGAGTTTGGCTCCAGCCAAGTGAC | |
| | TGGCAATACATTTGCAACACCATGAGCCAGCTTAACTTGGT | |
| | ATCACTTGATCAAGAAATATTCAATGTAGTGCTGAAAACTG | |
| | TTACAGAGCAAGACTTAGGAGGTCAAGCTATAAAAATATAC | |
| | AACAATGACCTTACAGCTTGCATGATGGTTGCAGTAGACTC | |
| | AAACAACATTTTGCCATACACACCTGCAGCAAACTCAATG | |
| | GAAACACTTGGTTTCTACCCCTGGAAACCAACCATAGCATC | |
| | ACCATACAGGTACTATTTTTGCGTTGACAGAGATCTTTCAG | |
| | TGACCTACGAAAATCAAGAAGGCACAGTTGAACATAATGT | |
| | GATGGGAACACCAAAAGGAATGAATTCTCAATTTTTTACCA | |
| | TTGAGAACACACAACAAATCACATTGCTCAGAACAGGGGA | |
| | CGAATTTGCCACAGGTACTTACTACTTTGACACAAATTCAG | |
| | TTAAACTCACACACACGTGGCAAACCAACCGTCAACTTGG | |
| | ACAGCCTCCACTGCTGTCAACCTTTCCTGAAGCTGACACT | |
| | GATGCAGGTACACTTACTGCTCAAGGGAGCAGACATGGAA | |
| | CAACACAAATGGGGGTTAACTGGGTGAGTGAAGCAATCAG | |
| | AACCAGACCTGCTCAAGTAGGATTTTGTCAACCACACAAT | |
| | GACTTTGAAGCCAGCAGAGCTGGACCATTTGCTGCCCCAA | |
| | AAGTTCCAGCAGATATTACTCAAGGAGTAGACAAAGAAGC | |
| | CAATGGCAGTGTTAGATACAGTTATGGCAAACAGCATGGTG | |
| | AAAATTGGGCTTCACATGGACCAGCACCAGAGCGCTACAC | |
| | ATGGGATGAAACAAGCTTTGGTTCAGGTAGAGACACCAAA | |
| | GATGGTTTTATTCAATCAGCACCACTAGTTGTTCCACCACC | |
| | ACTAAATGGCATTCTTACAAATGCAAACCCTATTGGGACTA | |
| | AAAATGACATTCATTTTTCAAATGTTTTTAACAGCTATGGTC | |
| | CACTAACTGCATTTTCACACCCAAGTCCTGTATACCCTCAA | |
| | GGACAAATATGGGACAAAGAACTAGATCTTGAACACAAAC | |
| | CTAGACTTCACATAACTGCTCCATTTGTTTGTAAAAACAAT | |
| | GCACCTGGACAAATGTTGGTTAGATTAGGACCAAACCTAA | |
| | CTGACCAATATGATCCAAACGGAGCCACACTTTCTAGAATT | |
| | GTTACATACGGTACATTTTTCTGGAAAGGAAAACTAACCAT | |
| | GAGAGCAAAACTTAGAGCTAACACCACTTGGAACCCAGTG | |
| | TACCAAGTAAGTGCTGAAGACAATGGCAACTCATACATGA | |
| | GTGTAACTAAATGGTTACCAACTGCTACTGGAAACATGCAG | |
| | TCTGTGCCGCTTATAACAAGACCTGTTGCTAGAAATACTTA | |
| | CTAACTCGAGGCATGCGGTACCAAGCTTGTCGAGAAGTAC | |
| | TAGAGGATCATAATCAGCCATACCACATTTGTAGAGGTTTTA | |
| | CTTGCTTTAAAAAACCTCCCACACCTCCCCCTGAACCTGAA | |
| | ACATAAAATGAATGCAATTGTTGTTGTTAACTTGTTTATTGC | |
| | AGCTTATAATGGTTACAAATAAAGCAATAGCATCACAAATTT | |
| | CACAAATAAAGCATTTTTTTCACTGCATTCTAGTTGTGGTTT | |
| | GTCCAAACTCATCAATGTATCTTATCATGTCTGGATC | |
| Exemplary | ATCATGGAGATAATTAAAATGATAACCATCTCGCAAATAAAT | SEQ ID |
| H-1PV | AAGTATTTTACTGTTTTCGTAACAGTTTTGTAATAAAAAAAC | NO: 138 |
| Construct 1 | CTATAAATACTCCGGACTACTGATACCGTCCCACTTTCGGG | |
| comprising | CGCTTACCTGCCGCCACGGCACCTCCAGCTAAAAGAGCTA | |
| a proto- | AAAGAGGCTACAAGTACCTGGGACCAGGGAACAGCCTTG | |
| parvovirus | ACCAAGGAGAACCAACCAACCCTTCTGACGCCGCTGCCAA | |
| variant | AGAACACGACGAAGCCTACGACCAATACATCAAATCTGGA | |
| VP1 capsid | AAAAATCCTTACCTGTACTTCTCTCCTGCTGATCAACGCTT | |
| coding | CATTGACCAAACCAAAGACGCCAAGGACTGGGGCGGCAA | |
| sequence | GGTTGGTCACTACTTTTTTAGAACCAAGCGAGCTTTTGCAC | |
| Ph-Kozak- | CTAAGCTTTCTACTGACTCTGAACCTGGCACTTCTGGTGTG | |
| RH1PV- | AGCAGACCTGGTAAACGAACTAAACCACCTGCTCACATTT | |
| VP1-ACG- | TTGTAAATCAAGCCAGAGCTAAAAAAAAACGCGCTTCTCT | |
| Del- | TGCTGCACAGCAGAGGACTCTGACAATGAGTGATGGCACC | |
| WVPPG | GAAACAAACCAACCAGACACTGGAATCGCTAATGCTAGAG | |
| | TTGAGCGATCAGCTGACGGAGGTGGAAGCTCTGGGGGTGG | |
| | GGGCTCTGGCGGGGGGGGATTGGTGTTTCTACTGGGACT | |
| | TATGATAATCAAACGACTTATAAGTTTTTGGGAGATGGATG | |
| | GGTAGAAATAACTGCACATGCTTCTAGACTTTTGCACTTGG | |
| | GAATGCCTCCTTCAGAAAACTACTGCCGCGTCACCGTTCAC | |
| | AATAATCAAACAACAGGACACGGAACTAAGGTAAAGGGA | |
| | AACATGGCCTATGATGACACACATCAACAAATTTGGACACC | |
| | ATGGAGCTTGGTAGATGCTAATGCTTGGGGAGTTTGGTTCC | |
| | AACCAAGTGACTGGCAGTTCATTCAAAACAGCATGGAATC | |
| | GCTGAATCTTGACTCATTGAGCCAAGAACTATTTAATGTAG | |
| | TAGTCAAAACAGTCACTGAACAACAAGGAGCTGGCCAAG | |
| | ATGCCATTAAAGTCTATAATAATGACTTGACGGCCTGTATGA | |
| | TGGTTGCTCTGGATAGTAACAACATACTGCCTTACACACCT | |
| | GCAGCTCAAACATCAGAAACACTTGGTTTCTACCCATGGA | |
| | AACCAACCGCACCAGCTCCTTACAGATACTACTTTTTCATG | |
| | CCTAGACAACTCAGTGTAACCTCTAGCAACTCTGCTGAAG | |
| | GAACTCAAATCACAGACACCATTGGAGAGCCACAGGCACT | |
| | AAACTCTCAATTTTTTACTATTGAGAACACCTTGCCTATTAC | |
| | TCTCCTGCGCACAGGTGATGAGTTTACAACTGGCACCTACA | |
| | TCTTTAACACTGACCCACTTAAACTTACTCACACATGGCAA | |
| | ACCAACAGACACTTGGGCATGCCTCCAAGAATAACTGACC | |
| | TACCAACATCAGATACAGCAACAGCATCACTAACTGCAAAT | |
| | GGAGACAGATTTGGATCAACACAAACACAGAATGTGAACT | |
| | ATGTCACAGAGGCTTTGCGCACCAGGCCTGCTCAGATTGG | |
| | CTTCATGCAACCTCATGACAACTTTGAAGCAAACAGAGGT | |
| | GGCCCATTTAAGGTTCCAGTGGTACCGCTAGACATAACAGC | |
| | TGGCGAGGACCATGATGCAAACGGAGCCATACGATTTAAC | |
| | TATGGCAAACAACATGGCGAAGATTGGGCCAAACAAGGAG | |
| | CAGCACCAGAAAGGTACACATGGGATGCAATTGATAGTGC | |
| | AGCTGGGAGGGACACAGCTAGATGCTTTGTACAAAGTGCA | |
| | CCAATATCTATTCCACCAAACCAAAACCAGATCTTGCAGCG | |
| | AGAAGACGCCATAGCTGGCAGAACTAACATGCATTATACTA | |
| | ATGTTTTTAACAGCTATGGTCCACTTAGTGCATTTCCTCATC | |
| | CAGATCCCATTTATCCAAATGGACAAATTTGGGACAAAGAA | |
| | TTGGACCTGGAACACAAACCTAGACTACACGTAACTGCAC | |
| | CATTTGTTTGTAAAAACAACCCACCAGGTCAACTATTTGTT | |
| | CGCTTGGGGCCTAATCTGACTGACCAATTTGACCCAAACA | |
| | GCACAACTGTTTCTCGCATTGTTACATATAGCACTTTTTACT | |
| | GGAAGGGTATTTTGAAATTCAAAGCCAAACTAAGACCAAA | |
| | TCTGACCTGGAATCCTGTATACCAAGCAACCACAGACTCTG | |
| | TTGCCAATTCTTACATGAATGTTAAGAAATGGCTCCCATCTG | |
| | CAACTGGCAACATGCACTCTGATCCATTGATTTGTAGACCT | |
| | GTGCCTCACATGACATACTAACTCGAGGCATGCGGTACCAA | |
| | GCTTGTCGAGAAGTACTAGAGGATCATAATCAGCCATACCA | |
| | CATTTGTAGAGGTTTTACTTGCTTTAAAAAACCTCCCACAC | |
| | CTCCCCCTGAACCTGAAACATAAAATGAATGCAATTGTTGT | |
| | TGTTAACTTGTTTATTGCAGCTTATAATGGTTACAAATAAAG | |
| | CAATAGCATCACAAATTTCACAAATAAAGCATTTTTTTCACT | |
| | GCATTCTAGTTGTGGTTTGTCCAAACTCATCAATGTATCTTA | |
| | TCATGTCTGGATC | |
| Exemplary | ATCATGGAGATAATTAAAATGATAACCATCTCGCAAATAAAT | SEQ ID |
| CuV | AAGTATTTTACTGTTTTCGTAACAGTTTTGTAATAAAAAAAC | NO: 139 |
| Construct 6 | CTATAAATACTCCGGACTACTGATACCGTCCCACTTTCGGG | |
| comprising | CGCTTACCTGCCGCCACGCCAGCTATTAGAAAAGCCAGAG | |
| a variant | GTTGGGTACCACCTGGATACAACTTCCTAGGACCCTTCAAT | |
| VP1 capsid | CAAGACTTCAACAAAGAACCAACTAATCCATCAGACAACG | |
| coding | CTGCAAAACAACACGATTTGGAATACAACAAACTAATCAA | |
| sequence | CCAAGGACACAATCCTTATTGGTACTACAACAAAGCTGAC | |
| Ph-Kozak- | GAAGACTTCATCAAAGCAACAGATCAAGCACCAGACTGGG | |
| CuV-VP1- | GAGGAAAATTTGGCAACTTCATCTTCAGAGCCAAAAAACA | |
| ACG | CATCGCTCCAGAACTGGCACCACCAGCAAAAAAGAAAAG | |
| | CAAAACCAAACACAGTGAACCAGAATTCAGCCACAAACA | |
| | CATCAAACCAGGCACCAAAAGAGGTAAGCCTTTTCATATTT | |
| | TTGTAAACCTTGCTAGAAAAAGAGCCCGCATGTCAGAACC | |
| | AGCTAATGATACAAATGAACAACCAGACAACTCCCCTGTT | |
| | GAACAGGGTGCTGGTCAAATTGGAGGAGGTGGAGGTGGA | |
| | GGTGGAAGCGGTGTCGGGCACAGCACTGGTGATTATAATA | |
| | ATAGGACTGAGTTTATTTATCATGGTGATGAAGTCACAATTA | |
| | TTTGCCACTCTACAAGACTGGTTCACATCAATATGTCAGAC | |
| | AGGGAAGACTACATCATCTATGAAACAGACAGAGGACCAC | |
| | TCTTTCCTACCACTCAGGACCTGCAGGGTAGAGACACTCTA | |
| | AATGACTCTTACCATGCCAAAGTAGAAACACCATGGAAAC | |
| | TACTCCATGCAAACAGCTGGGGCTGCTGGTTTTCACCAGC | |
| | AGACTTCCAACAAATGATCACCACATGCAGAGACATAGCA | |
| | CCAATAAAAATGCACCAAAAAATAGAAAACATTGTCATCA | |
| | AAACAGTCAGTAAAACAGGCACAGGAGAAACAGAAACAA | |
| | CCAACTACAACAATGACCTCACAGCACTCCTACAAATTGC | |
| | ACAAGACAACAGTAACCTACTACCATGGGCTGCAGATAAC | |
| | TTTTATATAGACTCAGTAGGTTACGTTCCATGGAGAGCATGC | |
| | AAACTACCAACCTACTGCTACCACGTAGACACTTGGAATAC | |
| | AATTGACATAAACCAAGCAGACACACCAAACCAATGGAGA | |
| | GAAATCAAAAAAGGCATCCAATGGGACAATATCCAATTCAC | |
| | ACCACTAGAAACTATGATAAACATTGACTTACTAAGAACAG | |
| | GAGATGCCTGGGAATCTGGTAACTACAATTTCCACACAAA | |
| | ACCAACAAACCTAGCTTACCATTGGCAATCACAAAGACAC | |
| | ACAGGCAGCTGTCACCCAACAGTAGCACCTCTAGTTGAAA | |
| | GAGGACAAGGAACCAACATACAATCAGTAAACTGTTGGCA | |
| | ATGGGGAGACAGAAACAATCCAAGCTCTGCATCAACCAGA | |
| | GTATCCAATATACATATTGGATACTCATTTCCAGAATGGCAA | |
| | ATCCACTACTCAACAGGAGGACCAGTAATTAATCCAGGCA | |
| | GTGCATTCTCACAAGCACCATGGGGCTCAACAACTGAAGG | |
| | CACCAGACTAACCCAAGGTGCATCTGAAAAAGCCATCTAT | |
| | GACTGGTCCCATGGAGATGACCAACCAGGAGCCAGAGAA | |
| | ACCTGGTGGCAAAACAACCAACATGTAACAGGACAAACTG | |
| | ACTGGGCACCAAAAAATGCACACACCTCAGAACTCAACA | |
| | ACAATGTACCAGCAGCCACACACTTCTGGAAAAACAGCTA | |
| | TCACAACACCTTCTCACCATTCACTGCAGTAGATGATCATG | |
| | GACCACAATATCCATGGGGAGCCATCTGGGGAAAATACCC | |
| | AGACACCACACACAAACCAATGATGTCAGCTCACGCACCA | |
| | TTCCTACTTCATGGACCACCTGGACAACTCTTTGTAAAACT | |
| | AGCACCAAACTATACAGACACACTTGACAACGGAGGTGTA | |
| | ACACATCCCAGAATCGTCACATATGGAACCTTCTGGTGGTC | |
| | AGGACAACTCATCTTTAAAGGAAAACTACGCACTCCAAGA | |
| | CAATGGAATACCTACAACCTACCAAGCCTAGACAAAAGAG | |
| | AAACCATGAAAAACACAGTACCAAATGAAGTTGGTCACTT | |
| | TGAACTACCATACATGCCAGGAAGATGTCTACCAAACTACA | |
| | CATTGTAACTCGAGGCATGCGGTACCAAGCTTGTCGAGAA | |
| | GTACTAGAGGATCATAATCAGCCATACCACATTTGTAGAGG | |
| | TTTTACTTGCTTTAAAAAACCTCCCACACCTCCCCCTGAAC | |
| | CTGAAACATAAAATGAATGCAATTGTTGTTGTTAACTTGTT | |
| | TATTGCAGCTTATAATGGTTACAAATAAAGCAATAGCATCAC | |
| | AAATTTCACAAATAAAGCATTTTTTTCACTGCATTCTAGTTG | |
| | TGGTTTGTCCAAACTCATCAATGTATCTTATCATGTCTGGAT | |
| | C | |
| Exemplary | ATCATGGAGATAATTAAAATGATAACCATCTCGCAAATAAAT | SEQ ID |
| MVM | AAGTATTTTACTGTTTTCGTAACAGTTTTGTAATAAAAAAAC | NO: 140 |
| Construct 2 | CTATAAATACTCCGGACTACTGATACCGTCCCACTTTCGGG | |
| comprising | CGCTTACCTGCCGCCACGGCGCCTCCAGCTAAAAGAGCTA | |
| a variant | AAAGAGGTTGGGTGCCTCCTGGCTACAAGTACCTGGGACC | |
| VP1 capsid | AGGGAACAGCCTTGACCAAGGAGAACCAACCAATCCATCT | |
| coding | GACGCCGCTGCCAAAGAGCACGACGAGGCCTACGATCAAT | |
| sequence | ACATCAAATCTGGAAAAAATCCTTACCTGTACTTCTCTGCT | |
| Ph-Kozak- | GCTGATCAACGCTTTATTGACCAAACCAAGGACGCCAAAG | |
| MVM-VP1- | ACTGGGGAGGCAAGGTTGGTCACTACTTTTTTAGAACCAA | |
| ACG | GCGCGCTTTTGCACCTAAGCTTGCTACTGACTCTGAACCTG | |
| | GAACTTCTGGTGTAAGCAGAGCTGGTAAACGCACTAGACC | |
| | ACCTGCTTACATTTTTATTAACCAAGCCAGAGCTAAAAAAA | |
| | AACTTACTTCTTCTGCTGCACAGCAAAGCAGTCAAACCAT | |
| | GAGTGATGGCACCAGCCAACCTGACAGCGGAAACGCTGTC | |
| | CACTCAGCTGCAAGAGTTGAACGAGCAGCTGACGGCCCTG | |
| | GAGGCTCTGGGGGTGGGGGCTCTGGCGGGGGTGGGGTTG | |
| | GTGTTTCTACTGGGTCTTATGATAATCAAACGCATTATAGAT | |
| | TCTTGGGTGACGGCTGGGTAGAAATTACTGCACTAGCAACT | |
| | AGACTAGTACATTTAAACATGCCTAAATCAGAAAACTATTG | |
| | CAGAATCAGAGTTCACAATACAACAGACACATCAGTCAAA | |
| | GGCAACATGGCAAAAGATGATGCTCATGAGCAAATTTGGA | |
| | CACCATGGAGCTTGGTGGATGCTAATGCTTGGGGAGTTTGG | |
| | CTCCAGCCAAGTGACTGGCAATACATTTGCAACACCATGA | |
| | GCCAGCTTAACTTGGTATCACTTGATCAAGAAATATTCAAT | |
| | GTAGTGCTGAAAACTGTTACAGAGCAAGACTTAGGAGGTC | |
| | AAGCTATAAAAATATACAACAATGACCTTACAGCTTGCATG | |
| | ATGGTTGCAGTAGACTCAAACAACATTTTGCCATACACACC | |
| | TGCAGCAAACTCAATGGAAACACTTGGTTTCTACCCCTGG | |
| | AAACCAACCATAGCATCACCATACAGGTACTATTTTTGCGT | |
| | TGACAGAGATCTTTCAGTGACCTACGAAAATCAAGAAGGC | |
| | ACAGTTGAACATAATGTGATGGGAACACCAAAAGGAATGA | |
| | ATTCTCAATTTTTTACCATTGAGAACACACAACAAATCACA | |
| | TTGCTCAGAACAGGGGACGAATTTGCCACAGGTACTTACT | |
| | ACTTTGACACAAATTCAGTTAAACTCACACACACGTGGCA | |
| | AACCAACCGTCAACTTGGACAGCCTCCACTGCTGTCAACC | |
| | TTTCCTGAAGCTGACACTGATGCAGGTACACTTACTGCTCA | |
| | AGGGAGCAGACATGGAACAACACAAATGGGGGTTAACTG | |
| | GGTGAGTGAAGCAATCAGAACCAGACCTGCTCAAGTAGGA | |
| | TTTTGTCAACCACACAATGACTTTGAAGCCAGCAGAGCTG | |
| | GACCATTTGCTGCCCCAAAAGTTCCAGCAGATATTACTCAA | |
| | GGAGTAGACAAAGAAGCCAATGGCAGTGTTAGATACAGTT | |
| | ATGGCAAACAGCATGGTGAAAATTGGGCTTCACATGGACC | |
| | AGCACCAGAGCGCTACACATGGGATGAAACAAGCTTTGGT | |
| | TCAGGTAGAGACACCAAAGATGGTTTTATTCAATCAGCACC | |
| | ACTAGTTGTTCCACCACCACTAAATGGCATTCTTACAAATG | |
| | CAAACCCTATTGGGACTAAAAATGACATTCATTTTTCAAAT | |
| | GTTTTTAACAGCTATGGTCCACTAACTGCATTTTCACACCC | |
| | AAGTCCTGTATACCCTCAAGGACAAATATGGGACAAAGAA | |
| | CTAGATCTTGAACACAAACCTAGACTTCACATAACTGCTCC | |
| | ATTTGTTTGTAAAAACAATGCACCTGGACAAATGTTGGTTA | |
| | GATTAGGACCAAACCTAACTGACCAATATGATCCAAACGGA | |
| | GCCACACTTTCTAGAATTGTTACATACGGTACATTTTTCTGG | |
| | AAAGGAAAACTAACCATGAGAGCAAAACTTAGAGCTAACA | |
| | CCACTTGGAACCCAGTGTACCAAGTAAGTGCTGAAGACAA | |
| | TGGCAACTCATACATGAGTGTAACTAAATGGTTACCAACTG | |
| | CTACTGGAAACATGCAGTCTGTGCCGCTTATAACAAGACCT | |
| | GTTGCTAGAAATACTTACTAACTCGAGGCATGCGGTACCAA | |
| | GCTTGTCGAGAAGTACTAGAGGATCATAATCAGCCATACCA | |
| | CATTTGTAGAGGTTTTACTTGCTTTAAAAAACCTCCCACAC | |
| | CTCCCCCTGAACCTGAAACATAAAATGAATGCAATTGTTGT | |
| | TGTTAACTTGTTTATTGCAGCTTATAATGGTTACAAATAAAG | |
| | CAATAGCATCACAAATTTCACAAATAAAGCATTTTTTTCACT | |
| | GCATTCTAGTTGTGGTTTGTCCAAACTCATCAATGTATCTTA | |
| | TCATGTCTGGATC | |
| Exemplary | ATCATGGAGATAATTAAAATGATAACCATCTCGCAAATAAAT | SEQ ID |
| H-1PV | AAGTATTTTACTGTTTTCGTAACAGTTTTGTAATAAAAAAAC | NO: 141 |
| Construct 2 | CTATAAATACTCCGGACTACTGATACCGTCCCACTTTCGGG | |
| comprising | CGCTTACCTGCCGCCACGGCACCTCCAGCTAAAAGAGCTA | |
| a variant | AAAGAGGTTGGGTGCCTCCTGGCTACAAGTACCTGGGACC | |
| VP1 capsid | AGGGAACAGCCTTGACCAAGGAGAACCAACCAACCCTTC | |
| coding | TGACGCCGCTGCCAAAGAACACGACGAAGCCTACGACCA | |
| sequence | ATACATCAAATCTGGAAAAAATCCTTACCTGTACTTCTCTCC | |
| Ph-Kozak- | TGCTGATCAACGCTTCATTGACCAAACCAAAGACGCCAAG | |
| RH1PV- | GACTGGGGCGGCAAGGTTGGTCACTACTTTTTTAGAACCA | |
| VP1-ACG | AGCGAGCTTTTGCACCTAAGCTTTCTACTGACTCTGAACCT | |
| | GGCACTTCTGGTGTGAGCAGACCTGGTAAACGAACTAAAC | |
| | CACCTGCTCACATTTTTGTAAATCAAGCCAGAGCTAAAAAA | |
| | AAACGCGCTTCTCTTGCTGCACAGCAGAGGACTCTGACAA | |
| | TGAGTGATGGCACCGAAACAAACCAACCAGACACTGGAAT | |
| | CGCTAATGCTAGAGTTGAGCGATCAGCTGACGGAGGTGGA | |
| | AGCTCTGGGGGTGGGGGCTCTGGCGGGGGTGGGATTGGTG | |
| | TTTCTACTGGGACTTATGATAATCAAACGACTTATAAGTTTT | |
| | TGGGAGATGGATGGGTAGAAATAACTGCACATGCTTCTAGA | |
| | CTTTTGCACTTGGGAATGCCTCCTTCAGAAAACTACTGCCG | |
| | CGTCACCGTTCACAATAATCAAACAACAGGACACGGAACT | |
| | AAGGTAAAGGGAAACATGGCCTATGATGACACACATCAAC | |
| | AAATTTGGACACCATGGAGCTTGGTAGATGCTAATGCTTGG | |
| | GGAGTTTGGTTCCAACCAAGTGACTGGCAGTTCATTCAAA | |
| | ACAGCATGGAATCGCTGAATCTTGACTCATTGAGCCAAGA | |
| | ACTATTTAATGTAGTAGTCAAAACAGTCACTGAACAACAAG | |
| | GAGCTGGCCAAGATGCCATTAAAGTCTATAATAATGACTTG | |
| | ACGGCCTGTATGATGGTTGCTCTGGATAGTAACAACATACT | |
| | GCCTTACACACCTGCAGCTCAAACATCAGAAACACTTGGT | |
| | TTCTACCCATGGAAACCAACCGCACCAGCTCCTTACAGATA | |
| | CTACTTTTTCATGCCTAGACAACTCAGTGTAACCTCTAGCA | |
| | ACTCTGCTGAAGGAACTCAAATCACAGACACCATTGGAGA | |
| | GCCACAGGCACTAAACTCTCAATTTTTTACTATTGAGAACA | |
| | CCTTGCCTATTACTCTCCTGCGCACAGGTGATGAGTTTACA | |
| | ACTGGCACCTACATCTTTAACACTGACCCACTTAAACTTAC | |
| | TCACACATGGCAAACCAACAGACACTTGGGCATGCCTCCA | |
| | AGAATAACTGACCTACCAACATCAGATACAGCAACAGCATC | |
| | ACTAACTGCAAATGGAGACAGATTTGGATCAACACAAACA | |
| | CAGAATGTGAACTATGTCACAGAGGCTTTGCGCACCAGGC | |
| | CTGCTCAGATTGGCTTCATGCAACCTCATGACAACTTTGAA | |
| | GCAAACAGAGGTGGCCCATTTAAGGTTCCAGTGGTACCGC | |
| | TAGACATAACAGCTGGCGAGGACCATGATGCAAACGGAGC | |
| | CATACGATTTAACTATGGCAAACAACATGGCGAAGATTGGG | |
| | CCAAACAAGGAGCAGCACCAGAAAGGTACACATGGGATG | |
| | CAATTGATAGTGCAGCTGGGAGGGACACAGCTAGATGCTT | |
| | TGTACAAAGTGCACCAATATCTATTCCACCAAACCAAAACC | |
| | AGATCTTGCAGCGAGAAGACGCCATAGCTGGCAGAACTAA | |
| | CATGCATTATACTAATGTTTTTAACAGCTATGGTCCACTTAG | |
| | TGCATTTCCTCATCCAGATCCCATTTATCCAAATGGACAAAT | |
| | TTGGGACAAAGAATTGGACCTGGAACACAAACCTAGACTA | |
| | CACGTAACTGCACCATTTGTTTGTAAAAACAACCCACCAG | |
| | GTCAACTATTTGTTCGCTTGGGGCCTAATCTGACTGACCAA | |
| | TTTGACCCAAACAGCACAACTGTTTCTCGCATTGTTACATA | |
| | TAGCACTTTTTACTGGAAGGGTATTTTGAAATTCAAAGCCA | |
| | AACTAAGACCAAATCTGACCTGGAATCCTGTATACCAAGCA | |
| | ACCACAGACTCTGTTGCCAATTCTTACATGAATGTTAAGAA | |
| | ATGGCTCCCATCTGCAACTGGCAACATGCACTCTGATCCAT | |
| | TGATTTGTAGACCTGTGCCTCACATGACATACTAACTCGAG | |
| | GCATGCGGTACCAAGCTTGTCGAGAAGTACTAGAGGATCAT | |
| | AATCAGCCATACCACATTTGTAGAGGTTTTACTTGCTTTAAA | |
| | AAACCTCCCACACCTCCCCCTGAACCTGAAACATAAAATG | |
| | AATGCAATTGTTGTTGTTAACTTGTTTATTGCAGCTTATAAT | |
| | GGTTACAAATAAAGCAATAGCATCACAAATTTCACAAATAA | |
| | AGCATTTTTTTCACTGCATTCTAGTTGTGGTTTGTCCAAACT | |
| | CATCAATGTATCTTATCATGTCTGGATC | |
| Exemplary | GACATTGATTATTGACTAGTTATTAATAGTAATCAATTACGG | SEQ ID |
| CPV | GGTCATTAGTTCATAGCCCATATATGGAGTTCCGCGTTACAT | NO: 142 |
| Construct 6 | AACTTACGGTAAATGGCCCGCCTGGCTGACCGCCCAACGA | |
| comprising | CCCCCGCCCATTGACGTCAATAATGACGTATGTTCCCATAGT | |
| a variant | AACGCCAATAGGGACTTTCCATTGACGTCAATGGGTGGACT | |
| VP2 capsid | ATTTACGGTAAACTGCCCACTTGGCAGTACATCAAGTGTAT | |
| coding | CATATGCCAAGTACGCCCCCTATTGACGTCAATGACGGTAA | |
| sequence | ATGGCCCGCCTGGCATTATGCCCAGTACATGACCTTATGGG | |
| CMV- | ACTTTCCTACTTGGCAGTACATCTACGTATTAGTCATCGCTA | |
| opt_CPV_ | TTACCATGGTGATGCGGTTTTGGCAGTACATCAATGGGCGT | |
| VP2 | GGATAGCGGTTTGACTCACGGGGATTTCCAAGTCTCCACCC | |
| | CATTGACGTCAATGGGAGTTTGTTTTGGCACCAAAATCAAC | |
| | GGGACTTTCCAAAATGTCGTAACAACTCCGCCCCATTGACG | |
| | CAAATGGGCGGTAGGCGTGTACGGTGGGAGGTCTATATAAG | |
| | CAGAGCTCTCTGGCTAACTAGAGAACCCACTGCTTACTGG | |
| | CTTATCGAAATTAATACGACTCACTATAGGGAGACCCAAGC | |
| | TTGGTACCGGACTCTAGAGGATCCGGTACTCGAGGAACTG | |
| | AAAAACCAGAAAGTTAACTGGTAAGTTTAGTCTTTTTGTCT | |
| | TTTATTTCAGGTCCCGGATCCGGTGGTGGTGCAAATCAAAG | |
| | AACTGCTCCTCAGTGGATGTTGCCTTTACTTCTAGGCCTGT | |
| | ACGGAAGTGTTACTTCTGCTCTAAAAGCTGCGGAATTGTAC | |
| | CCGCGGTTGAGGAACCTGTTAAGATGAGCGACGGCGCCGT | |
| | GCAGCCCGACGGCGGCCAGCCCGCCGTGCGCAACGAGCG | |
| | CGCCACCGGCAGCGGCAACGGCAGCGGCGGCGGCGGCGG | |
| | CGGCGGCAGCGGCGGCGTGGGCATCAGCACCGGCACCTTC | |
| | AACAACCAGACCGAGTTCAAGTTCCTGGAGAACGGCTGG | |
| | GTGGAGATCACCGCCAACAGCAGCCGCCTGGTGCACCTGA | |
| | ACATGCCCGAGAGCGAGAACTACCGCCGCGTGGTGGTGAA | |
| | CAACATGGACAAGACCGCCGTGAACGGCAACATGGCCCTG | |
| | GACGACATCCACGCCCAGATCGTGACCCCCTGGAGCCTGG | |
| | TGGACGCCAACGCCTGGGGCGTGTGGTTCAACCCCGGCGA | |
| | CTGGCAGCTGATCGTGAACACCATGAGCGAGCTGCACCTG | |
| | GTGAGCTTCGAGCAGGAGATCTTCAACGTGGTGCTGAAGA | |
| | CCGTGAGCGAGAGCGCCACCCAGCCCCCCACCAAGGTGTA | |
| | CAACAACGACCTGACCGCCAGCCTGATGGTGGCCCTGGAC | |
| | AGCAACAACACCATGCCCTTCACCCCCGCCGCCATGCGCA | |
| | GCGAGACCCTGGGCTTCTACCCCTGGAAGCCCACCATCCC | |
| | CACCCCCTGGCGCTACTACTTCCAGTGGGACCGCACCCTG | |
| | ATCCCCAGCCACACCGGCACCAGCGGCACCCCCACCAACA | |
| | TCTACCACGGCACCGACCCCGACGACGTGCAGTTCTACAC | |
| | CATCGAGAACAGCGTGCCCGTGCACCTGCTGCGCACCGGC | |
| | GACGAGTTCGCCACCGGCACCTTCTTCTTCGACTGCAAGC | |
| | CCTGCCGCCTGACCCACACCTGGCAGACCAACCGCGCCCT | |
| | GGGCCTGCCCCCCTTCCTGAACAGCCTGCCCCAGAGCGAG | |
| | GGCGCCACCAACTTCGGCGACATCGGCGTGCAGCAGGACA | |
| | AGCGCCGCGGCGTGACCCAGATGGGCAACACCAACTACAT | |
| | CACCGAGGCCACCATCATGCGCCCCGCCGAGGTGGGCTAC | |
| | AGCGCCCCCTACTACAGCTTCGAGGCCAGCACCCAGGGCC | |
| | CCTTCAAGACCCCCATCGCCGCCGGCCGCGGCGGCGCCCA | |
| | GACCTACGAGAACCAGGCCGCCGACGGCGACCCCCGCTAC | |
| | GCCTTCGGCCGCCAGCACGGCCAGAAGACCACCACCACC | |
| | GGCGAGACCCCCGAGCGCTTCACCTACATCGCCCACCAGG | |
| | ACACCGGCCGCTACCCCGAGGGCGACTGGATCCAGAACAT | |
| | CAACTTCAACCTGCCCGTGACCAACGACAACGTGCTGCTG | |
| | CCCACCGACCCCATCGGCGGCAAGACCGGCATCAACTACA | |
| | CCAACATCTTCAACACCTACGGCCCCCTGACCGCCCTGAA | |
| | CAACGTGCCCCCCGTGTACCCCAACGGCCAGATCTGGGAC | |
| | AAGGAGTTCGACACCGACCTGAAGCCCCGCCTGCACGTGA | |
| | ACGCCCCCTTCGTGTGCCAGAACAACTGCCCCGGCCAGCT | |
| | GTTCGTGAAGGTGGCCCCCAACCTGACCAACGAGTACGAC | |
| | CCCGACGCCAGCGCCAACATGAGCCGCATCGTGACCTACA | |
| | GCGACTTCTGGTGGAAGGGCAAGCTGGTGTTCAAGGCCAA | |
| | GCTGCGCGCCAGCCACACCTGGAACCCCATCCAGCAGATG | |
| | AGCATCAACGTGGACAACCAGTTCAACTACGTGCCCAGCA | |
| | ACATCGGCGGCATGAAGATCGTGTACGAGAAGAGCCAGCT | |
| | GGCCCCCCGCAAGCTGTACTAATAACTCGAGCATGCATCTA | |
| | GAGATCTAGATAGAGCTCGCTGATCAGCCTCGACTGTGCCT | |
| | TCTAGTTGCCAGCCATCTGTTGTTTGCCCCTCCCCCGTGCC | |
| | TTCCTTGACCCTGGAAGGTGCCACTCCCACTGTCCTTTCCT | |
| | AATAAAATGAGGAAATTGCATCGCATTGTCTGAGTAGGTGT | |
| | CATTCTATTCTGGGGGGTGGGGTGGGGCAGGACAGCAAGG | |
| | GGGAGGATTGGGAAGACAATAGCAGGCATGCTGGGGA | |
| Exemplary | GACATTGATTATTGACTAGTTATTAATAGTAATCAATTACGG | SEQ ID |
| CuV | GGTCATTAGTTCATAGCCCATATATGGAGTTCCGCGTTACAT | NO: 143 |
| Construct 7 | AACTTACGGTAAATGGCCCGCCTGGCTGACCGCCCAACGA | |
| comprising | CCCCCGCCCATTGACGTCAATAATGACGTATGTTCCCATAGT | |
| a variant | AACGCCAATAGGGACTTTCCATTGACGTCAATGGGTGGACT | |
| VP2 capsid | ATTTACGGTAAACTGCCCACTTGGCAGTACATCAAGTGTAT | |
| coding | CATATGCCAAGTACGCCCCCTATTGACGTCAATGACGGTAA | |
| sequence | ATGGCCCGCCTGGCATTATGCCCAGTACATGACCTTATGGG | |
| CMV- | ACTTTCCTACTTGGCAGTACATCTACGTATTAGTCATCGCTA | |
| opt_CuV_ | TTACCATGGTGATGCGGTTTTGGCAGTACATCAATGGGCGT | |
| VP2 | GGATAGCGGTTTGACTCACGGGGATTTCCAAGTCTCCACCC | |
| | CATTGACGTCAATGGGAGTTTGTTTTGGCACCAAAATCAAC | |
| | GGGACTTTCCAAAATGTCGTAACAACTCCGCCCCATTGACG | |
| | CAAATGGGCGGTAGGCGTGTACGGTGGGAGGTCTATATAAG | |
| | CAGAGCTCTCTGGCTAACTAGAGAACCCACTGCTTACTGG | |
| | CTTATCGAAATTAATACGACTCACTATAGGGAGACCCAAGC | |
| | TTGGTACCGGACTCTAGAGGATCCGGTACTCGAGGAACTG | |
| | AAAAACCAGAAAGTTAACTGGTAAGTTTAGTCTTTTTGTCT | |
| | TTTATTTCAGGTCCCGGATCCGGTGGTGGTGCAAATCAAAG | |
| | AACTGCTCCTCAGTGGATGTTGCCTTTACTTCTAGGCCTGT | |
| | ACGGAAGTGTTACTTCTGCTCTAAAAGCTGCGGAATTGTAC | |
| | CCGCGGTTGAGGAACCTGTTAAGATGAGCGAGCCCGCCAA | |
| | CGACACCAACGAGCAGCCCGACAACAGCCCCGTGGAGCA | |
| | GGGCGCCGGCCAGATCGGCGGCGGCGGCGGCGGCGGCGG | |
| | CAGCGGCGTGGGCCACAGCACCGGCGACTACAACAACCG | |
| | CACCGAGTTCATCTACCACGGCGACGAGGTGACCATCATCT | |
| | GCCACAGCACCCGCCTGGTGCACATCAACATGAGCGACCG | |
| | CGAGGACTACATCATCTACGAGACCGACCGCGGCCCCCTG | |
| | TTCCCCACCACCCAGGACCTGCAGGGCCGCGACACCCTGA | |
| | ACGACAGCTACCACGCCAAGGTGGAGACCCCCTGGAAGCT | |
| | GCTGCACGCCAACAGCTGGGGCTGCTGGTTCAGCCCCGCC | |
| | GACTTCCAGCAGATGATCACCACCTGCCGCGACATCGCCC | |
| | CCATCAAGATGCACCAGAAGATCGAGAACATCGTGATCAA | |
| | GACCGTGAGCAAGACCGGCACCGGCGAGACCGAGACCAC | |
| | CAACTACAACAACGACCTGACCGCCCTGCTGCAGATCGCC | |
| | CAGGACAACAGCAACCTGCTGCCCTGGGCCGCCGACAACT | |
| | TCTACATCGACAGCGTGGGCTACGTGCCCTGGCGCGCCTG | |
| | CAAGCTGCCCACCTACTGCTACCACGTGGACACCTGGAAC | |
| | ACCATCGACATCAACCAGGCCGACACCCCCAACCAGTGGC | |
| | GCGAGATCAAGAAGGGCATCCAGTGGGACAACATCCAGTT | |
| | CACCCCCCTGGAGACCATGATCAACATCGACCTGCTGCGC | |
| | ACCGGCGACGCCTGGGAGAGCGGCAACTACAACTTCCACA | |
| | CCAAGCCCACCAACCTGGCCTACCACTGGCAGAGCCAGCG | |
| | CCACACCGGCAGCTGCCACCCCACCGTGGCCCCCCTGGTG | |
| | GAGCGCGGCCAGGGCACCAACATCCAGAGCGTGAACTGC | |
| | TGGCAGTGGGGCGACCGCAACAACCCCAGCAGCGCCAGC | |
| | ACCCGCGTGAGCAACATCCACATCGGCTACAGCTTCCCCG | |
| | AGTGGCAGATCCACTACAGCACCGGCGGCCCCGTGATCAA | |
| | CCCCGGCAGCGCCTTCAGCCAGGCCCCCTGGGGCAGCACC | |
| | ACCGAGGGCACCCGCCTGACCCAGGGCGCCAGCGAGAAG | |
| | GCCATCTACGACTGGAGCCACGGCGACGACCAGCCCGGCG | |
| | CCCGCGAGACCTGGTGGCAGAACAACCAGCACGTGACCG | |
| | GCCAGACCGACTGGGCCCCCAAGAACGCCCACACCAGCG | |
| | AGCTGAACAACAACGTGCCCGCCGCCACCCACTTCTGGAA | |
| | GAACAGCTACCACAACACCTTCAGCCCCTTCACCGCCGTG | |
| | GACGACCACGGCCCCCAGTACCCCTGGGGCGCCATCTGGG | |
| | GCAAGTACCCCGACACCACCCACAAGCCCATGATGAGCGC | |
| | CCACGCCCCCTTCCTGCTGCACGGCCCCCCCGGCCAGCTG | |
| | TTCGTGAAGCTGGCCCCCAACTACACCGACACCCTGGACA | |
| | ACGGCGGCGTGACCCACCCCCGCATCGTGACCTACGGCAC | |
| | CTTCTGGTGGAGCGGCCAGCTGATCTTCAAGGGCAAGCTG | |
| | CGCACCCCCCGCCAGTGGAACACCTACAACCTGCCCAGCC | |
| | TGGACAAGCGCGAGACCATGAAGAACACCGTGCCCAACG | |
| | AGGTGGGCCACTTCGAGCTGCCCTACATGCCCGGCCGCTG | |
| | CCTGCCCAACTACACCCTGTAATAACTCGAGCATGCATCTA | |
| | GAGATCTAGATAGAGCTCGCTGATCAGCCTCGACTGTGCCT | |
| | TCTAGTTGCCAGCCATCTGTTGTTTGCCCCTCCCCCGTGCC | |
| | TTCCTTGACCCTGGAAGGTGCCACTCCCACTGTCCTTTCCT | |
| | AATAAAATGAGGAAATTGCATCGCATTGTCTGAGTAGGTGT | |
| | CATTCTATTCTGGGGGGTGGGGTGGGGCAGGACAGCAAGG | |
| | GGGAGGATTGGGAAGACAATAGCAGGCATGCTGGGGA | |
| Exemplary | GACATTGATTATTGACTAGTTATTAATAGTAATCAATTACGG | SEQ ID |
| CPV | GGTCATTAGTTCATAGCCCATATATGGAGTTCCGCGTTACAT | NO: 148 |
| Construct | AACTTACGGTAAATGGCCCGCCTGGCTGACCGCCCAACGA | |
| comprising | CCCCCGCCCATTGACGTCAATAATGACGTATGTTCCCATAGT | |
| a variant | AACGCCAATAGGGACTTTCCATTGACGTCAATGGGTGGACT | |
| VP1 capsid | ATTTACGGTAAACTGCCCACTTGGCAGTACATCAAGTGTAT | |
| coding | CATATGCCAAGTACGCCCCCTATTGACGTCAATGACGGTAA | |
| sequence | ATGGCCCGCCTGGCATTATGCCCAGTACATGACCTTATGGG | |
| CMV- | ACTTTCCTACTTGGCAGTACATCTACGTATTAGTCATCGCTA | |
| codopt- | TTACCATGGTGATGCGGTTTTGGCAGTACATCAATGGGCGT | |
| CPV-VP1- | GGATAGCGGTTTGACTCACGGGGATTTCCAAGTCTCCACCC | |
| AAV2_Rep- | CATTGACGTCAATGGGAGTTTGTTTTGGCACCAAAATCAAC | |
| Kan | GGGACTTTCCAAAATGTCGTAACAACTCCGCCCCATTGACG | |
| | CAAATGGGCGGTAGGCGTGTACGGTGGGAGGTCTATATAAG | |
| | CAGAGCTCTCTGGCTAACTAGAGAACCCACTGCTTACTGG | |
| | CTTATCGAAATTAATACGACTCACTATAGGGAGACCCAAGC | |
| | TTGGTACCGGACTCTAGAGGATCCGGTACTCGAGGAACTG | |
| | AAAAACCAGAAAGTTAACTGGTAAGTTTAGTCTTTTTGTCT | |
| | TTTATTTCAGGTCCCGGATCCGGTGGTGGTGCAAATCAAAG | |
| | AACTGCTCCTCAGTGGATGTTGCCTTTACTTCTAGGCCTGT | |
| | ACGGAAGTGTTACTTCTGCTCTAAAAGCTGCGGAATTGTAC | |
| | CCGCGGAAGCTTCCTAGGCCGCCACCATGGCCCCCCCCGC | |
| | CAAGCGCGCCCGCCGCGGCCTGGTGCCCCCCGGCTACAAG | |
| | TACCTGGGCCCCGGCAACAGCCTGGACCAGGGCGAGCCCA | |
| | CCAACCCCAGCGACGCCGCCGCCAAGGAGCACGACGAGG | |
| | CCTACGCCGCCTACCTGCGCAGCGGCAAGAACCCCTACCT | |
| | GTACTTCAGCCCCGCCGACCAGCGCTTCATCGACCAGACC | |
| | AAGGACGCCAAGGACTGGGGCGGCAAGATCGGCCACTAC | |
| | TTCTTCCGCGCCAAGAAGGCCATCGCCCCCGTGCTGACCG | |
| | ACACCCCCGACCACCCCAGCACCAGCCGCCCCACCAAGCC | |
| | CACCAAGCGCAGCAAGCCCCCCCCCCACATCTTCATCAAC | |
| | CTGGCCAAGAAGAAGAAGGCCGGCGCCGGCCAGGTGAAG | |
| | CGCGACAACCTGGCCCCCATGAGCGACGGCGCCGTGCAGC | |
| | CCGACGGCGGCCAGCCCGCCGTGCGCAACGAGCGCGCCA | |
| | CCGGCAGCGGCAACGGCAGCGGCGGCGGCGGCGGCGGCG | |
| | GCAGCGGCGGCGTGGGCATCAGCACCGGCACCTTCAACAA | |
| | CCAGACCGAGTTCAAGTTCCTGGAGAACGGCTGGGTGGA | |
| | GATCACCGCCAACAGCAGCCGCCTGGTGCACCTGAACATG | |
| | CCCGAGAGCGAGAACTACCGCCGCGTGGTGGTGAACAAC | |
| | ATGGACAAGACCGCCGTGAACGGCAACATGGCCCTGGACG | |
| | ACATCCACGCCCAGATCGTGACCCCCTGGAGCCTGGTGGA | |
| | CGCCAACGCCTGGGGCGTGTGGTTCAACCCCGGCGACTGG | |
| | CAGCTGATCGTGAACACCATGAGCGAGCTGCACCTGGTGA | |
| | GCTTCGAGCAGGAGATCTTCAACGTGGTGCTGAAGACCGT | |
| | GAGCGAGAGCGCCACCCAGCCCCCCACCAAGGTGTACAA | |
| | CAACGACCTGACCGCCAGCCTGATGGTGGCCCTGGACAGC | |
| | AACAACACCATGCCCTTCACCCCCGCCGCCATGCGCAGCG | |
| | AGACCCTGGGCTTCTACCCCTGGAAGCCCACCATCCCCAC | |
| | CCCCTGGCGCTACTACTTCCAGTGGGACCGCACCCTGATCC | |
| | CCAGCCACACCGGCACCAGCGGCACCCCCACCAACATCTA | |
| | CCACGGCACCGACCCCGACGACGTGCAGTTCTACACCATC | |
| | GAGAACAGCGTGCCCGTGCACCTGCTGCGCACCGGCGAC | |
| | GAGTTCGCCACCGGCACCTTCTTCTTCGACTGCAAGCCCT | |
| | GCCGCCTGACCCACACCTGGCAGACCAACCGCGCCCTGGG | |
| | CCTGCCCCCCTTCCTGAACAGCCTGCCCCAGAGCGAGGGC | |
| | GCCACCAACTTCGGCGACATCGGCGTGCAGCAGGACAAGC | |
| | GCCGCGGCGTGACCCAGATGGGCAACACCAACTACATCAC | |
| | CGAGGCCACCATCATGCGCCCCGCCGAGGTGGGCTACAGC | |
| | GCCCCCTACTACAGCTTCGAGGCCAGCACCCAGGGCCCCT | |
| | TCAAGACCCCCATCGCCGCCGGCCGCGGCGGCGCCCAGAC | |
| | CTACGAGAACCAGGCCGCCGACGGCGACCCCCGCTACGCC | |
| | TTCGGCCGCCAGCACGGCCAGAAGACCACCACCACCGGC | |
| | GAGACCCCCGAGCGCTTCACCTACATCGCCCACCAGGACA | |
| | CCGGCCGCTACCCCGAGGGCGACTGGATCCAGAACATCAA | |
| | CTTCAACCTGCCCGTGACCAACGACAACGTGCTGCTGCCC | |
| | ACCGACCCCATCGGCGGCAAGACCGGCATCAACTACACCA | |
| | ACATCTTCAACACCTACGGCCCCCTGACCGCCCTGAACAA | |
| | CGTGCCCCCCGTGTACCCCAACGGCCAGATCTGGGACAAG | |
| | GAGTTCGACACCGACCTGAAGCCCCGCCTGCACGTGAACG | |
| | CCCCCTTCGTGTGCCAGAACAACTGCCCCGGCCAGCTGTT | |
| | CGTGAAGGTGGCCCCCAACCTGACCAACGAGTACGACCCC | |
| | GACGCCAGCGCCAACATGAGCCGCATCGTGACCTACAGCG | |
| | ACTTCTGGTGGAAGGGCAAGCTGGTGTTCAAGGCCAAGCT | |
| | GCGCGCCAGCCACACCTGGAACCCCATCCAGCAGATGAGC | |
| | ATCAACGTGGACAACCAGTTCAACTACGTGCCCAGCAACA | |
| | TCGGCGGCATGAAGATCGTGTACGAGAAGAGCCAGCTGGC | |
| | CCCCCGCAAGCTGTACTAATAACTCGAGCATGCATCTAGAG | |
| | GTACATCTAGATAGAGCTCGCTGATCAGCCTCGACTGTGCC | |
| | TTCTAGTTGCCAGCCATCTGTTGTTTGCCCCTCCCCCGTGC | |
| | CTTCCTTGACCCTGGAAGGTGCCACTCCCACTGTCCTTTCC | |
| | TAATAAAATGAGGAAATTGCATCGCATTGTCTGAGTAGGTG | |
| | TCATTCTATTCTGGGGGGTGGGGGGGGCAGGACAGCAAG | |
| | GGGGAGGATTGGGAAGACAATAGCAGGCATGCTGGGGA | |
| Exemplary | CATGGAGATAATTAAAATGATAACCATCTCGCAAATAAATAA | SEQ ID |
| CPV | GTATTTTACTGTTTTCGTAACAGTTTTGTAATAAAAAAACCT | NO: 149 |
| construct 8 | ATAAAATTCCGGATTATTCATACCGTCCCACCATCGGGCGCG | |
| comprising | GATCTGCCGCCCTGGCACCTCCGGCAAAGAGAGCCAGGAG | |
| a proto- | AGGATATAAATATCTTGGGCCTGGGAACAGTCTTGACCAAG | |
| parvovirus | GAGAACCAACTAACCCTTCTGACGCCGCTGCAAAAGAACA | |
| variant | CGACGAAGCTTACGCTGCTTATCTTCGCTCTGGTAAAAACC | |
| VP1 capsid | CATACTTATATTTCTCGCCAGCAGATCAACGCTTTATAGATC | |
| coding | AAACTAAGGACGCTAAAGATTGGGGGGGGAAAATAGGAC | |
| sequence | ATTATTTTTTTAGAGCTAAAAAGGCAATTGCTCCAGTATTAA | |
| Ph-Kozak- | CTGATACACCAGATCATCCATCAACATCAAGACCAACAAAA | |
| CPV-VP1- | CCAACTAAAAGAAGTAAACCACCACCTCATATTTTCATCAA | |
| CTG-del- | TCTTGCAAAAAAAAAAAAAGCCGGTGCAGGACAAGTAAA | |
| LVPPG | AAGAGACAATCTTGCACCAATGAGTGATGGAGCAGTTCAA | |
| | CCAGACGGTGGTCAACCTGCTGTCAGAAATGAAAGAGCTA | |
| | CAGGATCTGGGAACGGGTCTGGAGGCGGGGGTGGTGGTG | |
| | GTTCTGGGGGTGTGGGGATTTCTACGGGTACTTTCAATAAT | |
| | CAGACGGAATTTAAATTTTTGGAAAACGGATGGGTGGAAA | |
| | TCACAGCAAACTCAAGCAGACTTGTACATTTAAATATGCCA | |
| | GAAAGTGAAAATTATAGAAGAGTGGTTGTAAATAATATGGA | |
| | TAAAACTGCAGTTAACGGAAACATGGCTTTAGATGATATTC | |
| | ATGCACAAATTGTAACACCTTGGTCATTGGTTGATGCAAAT | |
| | GCTTGGGGAGTTTGGTTTAATCCAGGAGATTGGCAACTAAT | |
| | TGTTAATACTATGAGTGAGTTGCATTTAGTTAGTTTTGAACA | |
| | AGAAATTTTTAATGTTGTTTTAAAGACTGTTTCAGAATCTG | |
| | CTACTCAGCCACCAACTAAAGTTTATAATAATGATTTAACTG | |
| | CATCATTGATGGTTGCATTAGATAGTAATAATACTATGCCATT | |
| | TACTCCAGCAGCTATGAGATCTGAGACATTGGGTTTTTATCC | |
| | ATGGAAACCAACCATACCAACTCCATGGAGATATTATTTTC | |
| | AATGGGATAGAACATTAATACCATCTCATACTGGAACTAGT | |
| | GGCACACCAACAAATATATACCATGGTACAGATCCAGATGA | |
| | TGTTCAATTTTATACTATTGAAAATTCTGTGCCAGTACACTT | |
| | ACTAAGAACAGGTGATGAATTTGCTACAGGAACATTTTTTT | |
| | TTGATTGTAAACCATGTAGACTAACACATACATGGCAAACA | |
| | AATAGAGCATTGGGCTTACCACCATTTCTAAATTCTTTGCCT | |
| | CAATCTGAAGGAGCTACTAACTTTGGTGATATAGGAGTTCA | |
| | ACAAGATAAAAGACGTGGTGTAACTCAAATGGGAAATACA | |
| | AACTATATTACTGAAGCTACTATTATGAGACCAGCTGAGGTT | |
| | GGTTATAGTGCACCATATTATTCTTTTGAGGCGTCTACACAA | |
| | GGGCCATTTAAAACACCTATTGCAGCAGGACGGGGGGGAG | |
| | CGCAAACATATGAAAATCAAGCAGCAGATGGTGATCCAAG | |
| | ATATGCATTTGGTAGACAACATGGTCAAAAAACTACCACAA | |
| | CAGGAGAAACACCTGAGAGATTTACATATATAGCACATCAA | |
| | GATACAGGAAGATATCCAGAAGGAGATTGGATTCAAAATAT | |
| | TAACTTTAACCTTCCTGTAACGAATGATAATGTATTGCTACC | |
| | AACAGATCCAATTGGAGGTAAAACAGGAATTAACTATACTA | |
| | ATATATTTAATACTTATGGTCCTTTAACTGCATTAAATAATGT | |
| | ACCACCAGTTTATCCAAATGGTCAAATTTGGGATAAAGAAT | |
| | TTGATACTGACTTAAAACCAAGACTTCATGTAAATGCACCA | |
| | TTTGTTTGTCAAAATAATTGTCCTGGTCAATTATTTGTAAAA | |
| | GTTGCGCCTAATTTAACAAATGAATATGATCCTGATGCATCT | |
| | GCTAATATGTCAAGAATTGTAACTTACTCAGATTTTTGGTGG | |
| | AAAGGTAAATTAGTATTTAAAGCTAAACTAAGAGCCTCTCA | |
| | TACTTGGAATCCAATTCAACAAATGAGTATTAATGTAGATAA | |
| | CCAATTTAACTATGTACCAAGTAATATTGGAGGTATGAAAAT | |
| | TGTATATGAAAAATCTCAACTAGCACCTAGAAAATTATATTA | |
| | ACTCGAGGCATGCGGTACCAAGCTTGTCGAGAAGTACTAG | |
| | AGGATCATAATCAGCCATACCACATTTGTAGAGGTTTTACTT | |
| | GCTTTAAAAAACCTCCCACACCTCCCCCTGAACCTGAAAC | |
| | ATAAAATGAATGCAATTGTTGTTGTTAACTTGTTTATTGCAG | |
| | CTTATAATGGTTACAAATAAAGCAATAGCATCACAAATTTCA | |
| | CAAATAAAGCATTTTTTTCACTGCATTCTAGTTGTGGTTTGT | |
| | CCAAACTCATCAATGTATCTTATCATGTCTGGATC | |
| Exemplary | CATGGAGATAATTAAAATGATAACCATCTCGCAAATAAATAA | SEQ ID |
| CPV | GTATTTTACTGTTTTCGTAACAGTTTTGTAATAAAAAAACCT | NO: 150 |
| construct 9 | ATAAAATTCCGGATTATTCATACCGTCCCACCATCGGGCGCG | |
| comprising | GATCTGCCGCCACGGCACCTCCGGCAAAGAGAGCCAGGA | |
| a proto- | GAGGATATAAATATCTTGGGCCTGGGAACAGTCTTGACCAA | |
| parvovirus | GGAGAACCAACTAACCCTTCTGACGCCGCTGCAAAAGAAC | |
| variant | ACGACGAAGCTTACGCTGCTTATCTTCGCTCTGGTAAAAAC | |
| VP1 capsid | CCATACTTATATTTCTCGCCAGCAGATCAACGCTTTATAGAT | |
| coding | CAAACTAAGGACGCTAAAGATTGGGGGGGGAAAATAGGAC | |
| sequence | ATTATTTTTTTAGAGCTAAAAAGGCAATTGCTCCAGTATTAA | |
| Ph-Kozak- | CTGATACACCAGATCATCCATCAACATCAAGACCAACAAAA | |
| CPV-VP1- | CCAACTAAAAGAAGTAAACCACCACCTCATATTTTCATCAA | |
| ACG-del- | TCTTGCAAAAAAAAAAAAAGCCGGTGCAGGACAAGTAAA | |
| LVPPG | AAGAGACAATCTTGCACCAATGAGTGATGGAGCAGTTCAA | |
| | CCAGACGGTGGTCAACCTGCTGTCAGAAATGAAAGAGCTA | |
| | CAGGATCTGGGAACGGGTCTGGAGGCGGGGGTGGTGGTG | |
| | GTTCTGGGGGTGTGGGGATTTCTACGGGTACTTTCAATAAT | |
| | CAGACGGAATTTAAATTTTTGGAAAACGGATGGGTGGAAA | |
| | TCACAGCAAACTCAAGCAGACTTGTACATTTAAATATGCCA | |
| | GAAAGTGAAAATTATAGAAGAGTGGTTGTAAATAATATGGA | |
| | TAAAACTGCAGTTAACGGAAACATGGCTTTAGATGATATTC | |
| | ATGCACAAATTGTAACACCTTGGTCATTGGTTGATGCAAAT | |
| | GCTTGGGGAGTTTGGTTTAATCCAGGAGATTGGCAACTAAT | |
| | TGTTAATACTATGAGTGAGTTGCATTTAGTTAGTTTTGAACA | |
| | AGAAATTTTTAATGTTGTTTTAAAGACTGTTTCAGAATCTG | |
| | CTACTCAGCCACCAACTAAAGTTTATAATAATGATTTAACTG | |
| | CATCATTGATGGTTGCATTAGATAGTAATAATACTATGCCATT | |
| | TACTCCAGCAGCTATGAGATCTGAGACATTGGGTTTTTATCC | |
| | ATGGAAACCAACCATACCAACTCCATGGAGATATTATTTTC | |
| | AATGGGATAGAACATTAATACCATCTCATACTGGAACTAGT | |
| | GGCACACCAACAAATATATACCATGGTACAGATCCAGATGA | |
| | TGTTCAATTTTATACTATTGAAAATTCTGTGCCAGTACACTT | |
| | ACTAAGAACAGGTGATGAATTTGCTACAGGAACATTTTTTT | |
| | TTGATTGTAAACCATGTAGACTAACACATACATGGCAAACA | |
| | AATAGAGCATTGGGCTTACCACCATTTCTAAATTCTTTGCCT | |
| | CAATCTGAAGGAGCTACTAACTTTGGTGATATAGGAGTTCA | |
| | ACAAGATAAAAGACGTGGTGTAACTCAAATGGGAAATACA | |
| | AACTATATTACTGAAGCTACTATTATGAGACCAGCTGAGGTT | |
| | GGTTATAGTGCACCATATTATTCTTTTGAGGCGTCTACACAA | |
| | GGGCCATTTAAAACACCTATTGCAGCAGGACGGGGGGGAG | |
| | CGCAAACATATGAAAATCAAGCAGCAGATGGTGATCCAAG | |
| | ATATGCATTTGGTAGACAACATGGTCAAAAAACTACCACAA | |
| | CAGGAGAAACACCTGAGAGATTTACATATATAGCACATCAA | |
| | GATACAGGAAGATATCCAGAAGGAGATTGGATTCAAAATAT | |
| | TAACTTTAACCTTCCTGTAACGAATGATAATGTATTGCTACC | |
| | AACAGATCCAATTGGAGGTAAAACAGGAATTAACTATACTA | |
| | ATATATTTAATACTTATGGTCCTTTAACTGCATTAAATAATGT | |
| | ACCACCAGTTTATCCAAATGGTCAAATTTGGGATAAAGAAT | |
| | TTGATACTGACTTAAAACCAAGACTTCATGTAAATGCACCA | |
| | TTTGTTTGTCAAAATAATTGTCCTGGTCAATTATTTGTAAAA | |
| | GTTGCGCCTAATTTAACAAATGAATATGATCCTGATGCATCT | |
| | GCTAATATGTCAAGAATTGTAACTTACTCAGATTTTTGGTGG | |
| | AAAGGTAAATTAGTATTTAAAGCTAAACTAAGAGCCTCTCA | |
| | TACTTGGAATCCAATTCAACAAATGAGTATTAATGTAGATAA | |
| | CCAATTTAACTATGTACCAAGTAATATTGGAGGTATGAAAAT | |
| | TGTATATGAAAAATCTCAACTAGCACCTAGAAAATTATATTA | |
| | ACTCGAGGCATGCGGTACCAAGCTTGTCGAGAAGTACTAG | |
| | AGGATCATAATCAGCCATACCACATTTGTAGAGGTTTTACTT | |
| | GCTTTAAAAAACCTCCCACACCTCCCCCTGAACCTGAAAC | |
| | ATAAAATGAATGCAATTGTTGTTGTTAACTTGTTTATTGCAG | |
| | CTTATAATGGTTACAAATAAAGCAATAGCATCACAAATTTCA | |
| | CAAATAAAGCATTTTTTTCACTGCATTCTAGTTGTGGTTTGT | |
| | CCAAACTCATCAATGTATCTTATCATGTCTGGATC | |
| Exemplary | CATGGAGATAATTAAAATGATAACCATCTCGCAAATAAATAA | SEQ ID |
| CPV | GTATTTTACTGTTTTCGTAACAGTTTTGTAATAAAAAAACCT | NO: 151 |
| construct 10 | ATAAAATTCCGGATTATTCATACCGTCCCACCATCGGGCGCG | |
| comprising | GATCTGCCGCCTTGGCACCTCCGGCAAAGAGAGCCAGGAG | |
| a proto- | AGGATATAAATATCTTGGGCCTGGGAACAGTCTTGACCAAG | |
| parvovirus | GAGAACCAACTAACCCTTCTGACGCCGCTGCAAAAGAACA | |
| variant | CGACGAAGCTTACGCTGCTTATCTTCGCTCTGGTAAAAACC | |
| VP1 capsid | CATACTTATATTTCTCGCCAGCAGATCAACGCTTTATAGATC | |
| coding | AAACTAAGGACGCTAAAGATTGGGGGGGGAAAATAGGAC | |
| sequence | ATTATTTTTTTAGAGCTAAAAAGGCAATTGCTCCAGTATTAA | |
| Ph-Kozak- | CTGATACACCAGATCATCCATCAACATCAAGACCAACAAAA | |
| CPV-VP1- | CCAACTAAAAGAAGTAAACCACCACCTCATATTTTCATCAA | |
| TTG-del- | TCTTGCAAAAAAAAAAAAAGCCGGTGCAGGACAAGTAAA | |
| LVPPG | AAGAGACAATCTTGCACCAATGAGTGATGGAGCAGTTCAA | |
| | CCAGACGGTGGTCAACCTGCTGTCAGAAATGAAAGAGCTA | |
| | CAGGATCTGGGAACGGGTCTGGAGGCGGGGGTGGTGGTG | |
| | GTTCTGGGGGTGTGGGGATTTCTACGGGTACTTTCAATAAT | |
| | CAGACGGAATTTAAATTTTTGGAAAACGGATGGGTGGAAA | |
| | TCACAGCAAACTCAAGCAGACTTGTACATTTAAATATGCCA | |
| | GAAAGTGAAAATTATAGAAGAGTGGTTGTAAATAATATGGA | |
| | TAAAACTGCAGTTAACGGAAACATGGCTTTAGATGATATTC | |
| | ATGCACAAATTGTAACACCTTGGTCATTGGTTGATGCAAAT | |
| | GCTTGGGGAGTTTGGTTTAATCCAGGAGATTGGCAACTAAT | |
| | TGTTAATACTATGAGTGAGTTGCATTTAGTTAGTTTTGAACA | |
| | AGAAATTTTTAATGTTGTTTTAAAGACTGTTTCAGAATCTG | |
| | CTACTCAGCCACCAACTAAAGTTTATAATAATGATTTAACTG | |
| | CATCATTGATGGTTGCATTAGATAGTAATAATACTATGCCATT | |
| | TACTCCAGCAGCTATGAGATCTGAGACATTGGGTTTTTATCC | |
| | ATGGAAACCAACCATACCAACTCCATGGAGATATTATTTTC | |
| | AATGGGATAGAACATTAATACCATCTCATACTGGAACTAGT | |
| | GGCACACCAACAAATATATACCATGGTACAGATCCAGATGA | |
| | TGTTCAATTTTATACTATTGAAAATTCTGTGCCAGTACACTT | |
| | ACTAAGAACAGGTGATGAATTTGCTACAGGAACATTTTTTT | |
| | TTGATTGTAAACCATGTAGACTAACACATACATGGCAAACA | |
| | AATAGAGCATTGGGCTTACCACCATTTCTAAATTCTTTGCCT | |
| | CAATCTGAAGGAGCTACTAACTTTGGTGATATAGGAGTTCA | |
| | ACAAGATAAAAGACGTGGTGTAACTCAAATGGGAAATACA | |
| | AACTATATTACTGAAGCTACTATTATGAGACCAGCTGAGGTT | |
| | GGTTATAGTGCACCATATTATTCTTTTGAGGCGTCTACACAA | |
| | GGGCCATTTAAAACACCTATTGCAGCAGGACGGGGGGGAG | |
| | CGCAAACATATGAAAATCAAGCAGCAGATGGTGATCCAAG | |
| | ATATGCATTTGGTAGACAACATGGTCAAAAAACTACCACAA | |
| | CAGGAGAAACACCTGAGAGATTTACATATATAGCACATCAA | |
| | GATACAGGAAGATATCCAGAAGGAGATTGGATTCAAAATAT | |
| | TAACTTTAACCTTCCTGTAACGAATGATAATGTATTGCTACC | |
| | AACAGATCCAATTGGAGGTAAAACAGGAATTAACTATACTA | |
| | ATATATTTAATACTTATGGTCCTTTAACTGCATTAAATAATGT | |
| | ACCACCAGTTTATCCAAATGGTCAAATTTGGGATAAAGAAT | |
| | TTGATACTGACTTAAAACCAAGACTTCATGTAAATGCACCA | |
| | TTTGTTTGTCAAAATAATTGTCCTGGTCAATTATTTGTAAAA | |
| | GTTGCGCCTAATTTAACAAATGAATATGATCCTGATGCATCT | |
| | GCTAATATGTCAAGAATTGTAACTTACTCAGATTTTTGGTGG | |
| | AAAGGTAAATTAGTATTTAAAGCTAAACTAAGAGCCTCTCA | |
| | TACTTGGAATCCAATTCAACAAATGAGTATTAATGTAGATAA | |
| | CCAATTTAACTATGTACCAAGTAATATTGGAGGTATGAAAAT | |
| | TGTATATGAAAAATCTCAACTAGCACCTAGAAAATTATATTA | |
| | ACTCGAGGCATGCGGTACCAAGCTTGTCGAGAAGTACTAG | |
| | AGGATCATAATCAGCCATACCACATTTGTAGAGGTTTTACTT | |
| | GCTTTAAAAAACCTCCCACACCTCCCCCTGAACCTGAAAC | |
| | ATAAAATGAATGCAATTGTTGTTGTTAACTTGTTTATTGCAG | |
| | CTTATAATGGTTACAAATAAAGCAATAGCATCACAAATTTCA | |
| | CAAATAAAGCATTTTTTTCACTGCATTCTAGTTGTGGTTTGT | |
| | CCAAACTCATCAATGTATCTTATCATGTCTGGATC | |
| Exemplary | CATGGAGATAATTAAAATGATAACCATCTCGCAAATAAATAA | SEQ ID |
| CPV | GTATTTTACTGTTTTCGTAACAGTTTTGTAATAAAAAAACCT | NO: 152 |
| construct 11 | ATAAAATTCCGGATTATTCATACCGTCCCACCATCGGGCGCG | |
| comprising | GATCTGCCGCCATCGCACCTCCGGCAAAGAGAGCCAGGAG | |
| a proto- | AGGATATAAATATCTTGGGCCTGGGAACAGTCTTGACCAAG | |
| parvovirus | GAGAACCAACTAACCCTTCTGACGCCGCTGCAAAAGAACA | |
| variant | CGACGAAGCTTACGCTGCTTATCTTCGCTCTGGTAAAAACC | |
| VP1 capsid | CATACTTATATTTCTCGCCAGCAGATCAACGCTTTATAGATC | |
| coding | AAACTAAGGACGCTAAAGATTGGGGGGGGAAAATAGGAC | |
| sequence | ATTATTTTTTTAGAGCTAAAAAGGCAATTGCTCCAGTATTAA | |
| Ph-Kozak- | CTGATACACCAGATCATCCATCAACATCAAGACCAACAAAA | |
| CPV-VP1- | CCAACTAAAAGAAGTAAACCACCACCTCATATTTTCATCAA | |
| ATC-del- | TCTTGCAAAAAAAAAAAAAGCCGGTGCAGGACAAGTAAA | |
| LVPPG | AAGAGACAATCTTGCACCAATGAGTGATGGAGCAGTTCAA | |
| | CCAGACGGTGGTCAACCTGCTGTCAGAAATGAAAGAGCTA | |
| | CAGGATCTGGGAACGGGTCTGGAGGCGGGGGTGGTGGTG | |
| | GTTCTGGGGGTGTGGGGATTTCTACGGGTACTTTCAATAAT | |
| | CAGACGGAATTTAAATTTTTGGAAAACGGATGGGTGGAAA | |
| | TCACAGCAAACTCAAGCAGACTTGTACATTTAAATATGCCA | |
| | GAAAGTGAAAATTATAGAAGAGTGGTTGTAAATAATATGGA | |
| | TAAAACTGCAGTTAACGGAAACATGGCTTTAGATGATATTC | |
| | ATGCACAAATTGTAACACCTTGGTCATTGGTTGATGCAAAT | |
| | GCTTGGGGAGTTTGGTTTAATCCAGGAGATTGGCAACTAAT | |
| | TGTTAATACTATGAGTGAGTTGCATTTAGTTAGTTTTGAACA | |
| | AGAAATTTTTAATGTTGTTTTAAAGACTGTTTCAGAATCTG | |
| | CTACTCAGCCACCAACTAAAGTTTATAATAATGATTTAACTG | |
| | CATCATTGATGGTTGCATTAGATAGTAATAATACTATGCCATT | |
| | TACTCCAGCAGCTATGAGATCTGAGACATTGGGTTTTTATCC | |
| | ATGGAAACCAACCATACCAACTCCATGGAGATATTATTTTC | |
| | AATGGGATAGAACATTAATACCATCTCATACTGGAACTAGT | |
| | GGCACACCAACAAATATATACCATGGTACAGATCCAGATGA | |
| | TGTTCAATTTTATACTATTGAAAATTCTGTGCCAGTACACTT | |
| | ACTAAGAACAGGTGATGAATTTGCTACAGGAACATTTTTTT | |
| | TTGATTGTAAACCATGTAGACTAACACATACATGGCAAACA | |
| | AATAGAGCATTGGGCTTACCACCATTTCTAAATTCTTTGCCT | |
| | CAATCTGAAGGAGCTACTAACTTTGGTGATATAGGAGTTCA | |
| | ACAAGATAAAAGACGTGGTGTAACTCAAATGGGAAATACA | |
| | AACTATATTACTGAAGCTACTATTATGAGACCAGCTGAGGTT | |
| | GGTTATAGTGCACCATATTATTCTTTTGAGGCGTCTACACAA | |
| | GGGCCATTTAAAACACCTATTGCAGCAGGACGGGGGGGAG | |
| | CGCAAACATATGAAAATCAAGCAGCAGATGGTGATCCAAG | |
| | ATATGCATTTGGTAGACAACATGGTCAAAAAACTACCACAA | |
| | CAGGAGAAACACCTGAGAGATTTACATATATAGCACATCAA | |
| | GATACAGGAAGATATCCAGAAGGAGATTGGATTCAAAATAT | |
| | TAACTTTAACCTTCCTGTAACGAATGATAATGTATTGCTACC | |
| | AACAGATCCAATTGGAGGTAAAACAGGAATTAACTATACTA | |
| | ATATATTTAATACTTATGGTCCTTTAACTGCATTAAATAATGT | |
| | ACCACCAGTTTATCCAAATGGTCAAATTTGGGATAAAGAAT | |
| | TTGATACTGACTTAAAACCAAGACTTCATGTAAATGCACCA | |
| | TTTGTTTGTCAAAATAATTGTCCTGGTCAATTATTTGTAAAA | |
| | GTTGCGCCTAATTTAACAAATGAATATGATCCTGATGCATCT | |
| | GCTAATATGTCAAGAATTGTAACTTACTCAGATTTTTGGTGG | |
| | AAAGGTAAATTAGTATTTAAAGCTAAACTAAGAGCCTCTCA | |
| | TACTTGGAATCCAATTCAACAAATGAGTATTAATGTAGATAA | |
| | CCAATTTAACTATGTACCAAGTAATATTGGAGGTATGAAAAT | |
| | TGTATATGAAAAATCTCAACTAGCACCTAGAAAATTATATTA | |
| | ACTCGAGGCATGCGGTACCAAGCTTGTCGAGAAGTACTAG | |
| | AGGATCATAATCAGCCATACCACATTTGTAGAGGTTTTACTT | |
| | GCTTTAAAAAACCTCCCACACCTCCCCCTGAACCTGAAAC | |
| | ATAAAATGAATGCAATTGTTGTTGTTAACTTGTTTATTGCAG | |
| | CTTATAATGGTTACAAATAAAGCAATAGCATCACAAATTTCA | |
| | CAAATAAAGCATTTTTTTCACTGCATTCTAGTTGTGGTTTGT | |
| | CCAAACTCATCAATGTATCTTATCATGTCTGGATC | |
| Exemplary | GTATACTCCGGAATATTAATAGATGCGAAACACGCACGGCG | SEQ ID |
| CPV | CGCGCACGCAGCTTAGCACAAACGCGTCGTTGCACGCGCC | NO: 153 |
| construct 12 | CACCGCTAACCGCAGGCCAATCGGTCGGCCGGCCTCATATC | |
| comprising | CGCTCACCAGCCGCGTCCTATCGGGCGCGGCTTCCGCGCC | |
| a proto- | CATTTTGAATAAATAAACGATAACGCCGTTGGTGGCGTGAG | |
| parvovirus | GCATGTAAAAGGTTACATCATTATCTTGTTCGCCATCCGGTT | |
| variant | GGTATAAATAGACGTTCATGTTGGTTTTTGTTTCAGTTGCAA | |
| VP1 capsid | GTTGGCTGCGGCGCGCGCAGCACCTTTGCTATTCCGGATTA | |
| coding | TTCATACCGTCCCACCATCGGGCGCGGATCTGCCTCCATGT | |
| sequence | CTGGCAACCAGTATACTGAGGAAGTTATGGAGGGAGTAAA | |
| CPV- | TTGGTTAAAGAAACATGCAGAAAATGAAGCATTTTCGTTTG | |
| OpiE1-NS2- | TTTTTAAATGTGACAACGTCCAACTAAATGGAAAGGATGTT | |
| CTG | CGCTGGAACAACTATACCAAACCAATTCAAAATGAAGAAC | |
| | TAACATCTTTAATTAGAGGAGCACAAACAGCAATGGATCAA | |
| | ACCGAAGAAGAAGAAATGGACTGGGAATCGGAAGTTGATA | |
| | GTCTCGCCAAAAAGTTGCAAAGACTTAGAGACACAAGCG | |
| | GCAAGCAATCCTCAGAGTCAAGACCAAGTTCTAACTCCTC | |
| | TGACTCCGGACGTAGTGGACCTTGCACTGGAACCGTGGAG | |
| | TACTCCAGATACGCCTATTGCAGAAACTGCAAATCAACAAT | |
| | CAAACCAACTTGGCGTTACTCACAAAGACGTGCAAGCGAG | |
| | TCCGACGTGGTCCGAAATAGAGGCAGACCTGAGAGCCATC | |
| | TTTACTTCCATCATCACCATCACCACTGAGAGCTCACTAGT | |
| | CGCGGCCGCTTTCGAATCTAGAGCCTGCAGTCTCGAGGCAT | |
| | GCGGTACCAAGCTTGTCGAGAAGTACTAGAGGATCATAATC | |
| | AGCCATACCACATTTGTAGAGGTTTTACTTGCTTTAAAAAA | |
| | CCTCCCACACCTCCCCCTGAACCTGAAACATAAAATGAATG | |
| | CAATTGTTGTTGTTAACTTGTTTATTGCAGCTTATAATGGTT | |
| | ACAAATAAAGCAATAGCATCACAAATTTCACAAATAAAGCA | |
| | TTTTTTTCACTGCATTCTAGTTGTGGTTTGTCCAAACTCATC | |
| | AATGTATCTTATCATGTCTGGATC | |
| Exemplary | GTATACTCCGGAATATTAATAGATGCGAAACACGCACGGCG | SEQ ID |
| CPV | CGCGCACGCAGCTTAGCACAAACGCGTCGTTGCACGCGCC | NO: 154 |
| construct 13 | CACCGCTAACCGCAGGCCAATCGGTCGGCCGGCCTCATATC | |
| comprising | CGCTCACCAGCCGCGTCCTATCGGGCGCGGCTTCCGCGCC | |
| a proto- | CATTTTGAATAAATAAACGATAACGCCGTTGGTGGCGTGAG | |
| parvovirus | GCATGTAAAAGGTTACATCATTATCTTGTTCGCCATCCGGTT | |
| variant | GGTATAAATAGACGTTCATGTTGGTTTTTGTTTCAGTTGCAA | |
| VP1 capsid | GTTGGCTGCGGCGCGCGCAGCACCTTTGCTATTCCGGATTA | |
| coding | TTCATACCGTCCCACCATCGGGCGCGGATCTGCCTCCCTGT | |
| sequence | CTGGCAACCAGTATACTGAGGAAGTTATGGAGGGAGTAAA | |
| CPV- | TTGGTTAAAGAAACATGCAGAAAATGAAGCATTTTCGTTTG | |
| OpiE1-NS2- | TTTTTAAATGTGACAACGTCCAACTAAATGGAAAGGATGTT | |
| CTG | CGCTGGAACAACTATACCAAACCAATTCAAAATGAAGAAC | |
| | TAACATCTTTAATTAGAGGAGCACAAACAGCAATGGATCAA | |
| | ACCGAAGAAGAAGAAATGGACTGGGAATCGGAAGTTGATA | |
| | GTCTCGCCAAAAAGTTGCAAAGACTTAGAGACACAAGCG | |
| | GCAAGCAATCCTCAGAGTCAAGACCAAGTTCTAACTCCTC | |
| | TGACTCCGGACGTAGTGGACCTTGCACTGGAACCGTGGAG | |
| | TACTCCAGATACGCCTATTGCAGAAACTGCAAATCAACAAT | |
| | CAAACCAACTTGGCGTTACTCACAAAGACGTGCAAGCGAG | |
| | TCCGACGTGGTCCGAAATAGAGGCAGACCTGAGAGCCATC | |
| | TTTACTTCTGAGAGCTCACTAGTCGCGGCCGCTTTCGAATC | |
| | TAGAGCCTGCAGTCTCGAGGCATGCGGTACCAAGCTTGTC | |
| | GAGAAGTACTAGAGGATCATAATCAGCCATACCACATTTGT | |
| | AGAGGTTTTACTTGCTTTAAAAAACCTCCCACACCTCCCCC | |
| | TGAACCTGAAACATAAAATGAATGCAATTGTTGTTGTTAAC | |
| | TTGTTTATTGCAGCTTATAATGGTTACAAATAAAGCAATAGC | |
| | ATCACAAATTTCACAAATAAAGCATTTTTTTCACTGCATTCT | |
| | AGTTGTGGTTTGTCCAAACTCATCAATGTATCTTATCATGTC | |
| | TGGATC | |
| Exemplary | CGCGTGGCCTCCGCGCCGGGTTTTGGCGCCTCCCGCGGGC | SEQ ID |
| CPV | GCCCCCCTCCTCACGGCGAGCGCTGCCACGTCAGACGAAG | NO: 155 |
| Construct | GGCGCAGGAGCGTTCCTGATCCTTCCGCCCGGACGCTCAG | |
| 14 | GACAGCGGCCCGCTGCTCATAAGACTCGGCCTTAGAACCC | |
| comprising | CAGTATCAGCAGAAGGACATTTTAGGACGGGACTTGGGTG | |
| a proto- | ACTCTAGGGCACTGGTTTTCTTTCCAGAGAGCGGAACAGG | |
| parvovirus | CGAGGAAAAGTAGTCCCTTCTCGGCGATTCTGCGGAGGGA | |
| variant | TCTCCGTGGGGCGGTGAACGCCGATGATTATATAAGGACGC | |
| VP1 capsid | GCCGGGTGTGGCACAGCTAGTTCCGTCGCAGCCGGGATTT | |
| coding | GGGTCGCGGTTCTTGTTTGTGGATCGCTGTGATCGTCACTT | |
| sequence | GGTGGTACCGGACTCTAGAGGATCCGGTACTCGAGGAACT | |
| UBC_CPV_ | GAAAAACCAGAAAGTTAACTGGTAAGTTTAGTCTTTTTGTC | |
| VP1_del- | TTTTATTTCAGGTCCCGGATCCGGTGGTGGTGCAAATCAAA | |
| LVPPG | GAACTGCTCCTCAGTGGATGTTGCCTTTACTTCTAGGCCTG | |
| | TAGGGAAGTGTTACTTCTGCTCTAAAAGCTGCGGAATTGTA | |
| | GCCGTTGAAGCTTCCTAGGCCGCCACCATGGCCCCCCCCG | |
| | CCAAGCGCGCCCGCCGCGGCTACAAGTACCTGGGCCCCGG | |
| | CAACAGCCTGGACCAGGGCGAGCCCACCAACCCCAGCGA | |
| | CGCCGCCGCCAAGGAGCACGACGAGGCCTACGCCGCCTAC | |
| | CTGCGCAGCGGCAAGAACCCCTACCTGTACTTCAGCCCCG | |
| | CCGACCAGCGCTTCATCGACCAGACCAAGGACGCCAAGG | |
| | ACTGGGGCGGCAAGATCGGCCACTACTTCTTCCGCGCCAA | |
| | GAAGGCCATCGCCCCCGTGCTGACCGACACCCCCGACCAC | |
| | CCCAGCACCAGCCGCCCCACCAAGCCCACCAAGCGCAGC | |
| | AAGCCCCCCCCCCACATCTTCATCAACCTGGCCAAGAAGA | |
| | AGAAGGCCGGCGCCGGCCAGGTGAAGCGCGACAACCTGG | |
| | CCCCCATGAGCGACGGCGCCGTGCAGCCCGACGGCGGCCA | |
| | GCCCGCCGTGCGCAACGAGCGCGCCACCGGCAGCGGCAA | |
| | CGGCAGCGGCGGCGGCGGCGGCGGCGGCAGCGGCGGCGT | |
| | GGGCATCAGCACCGGCACCTTCAACAACCAGACCGAGTTC | |
| | AAGTTCCTGGAGAACGGCTGGGTGGAGATCACCGCCAACA | |
| | GCAGCCGCCTGGTGCACCTGAACATGCCCGAGAGCGAGA | |
| | ACTACCGCCGCGTGGTGGTGAACAACATGGACAAGACCGC | |
| | CGTGAACGGCAACATGGCCCTGGACGACATCCACGCCCAG | |
| | ATCGTGACCCCCTGGAGCCTGGTGGACGCCAACGCCTGGG | |
| | GCGTGTGGTTCAACCCCGGCGACTGGCAGCTGATCGTGAA | |
| | CACCATGAGCGAGCTGCACCTGGTGAGCTTCGAGCAGGAG | |
| | ATCTTCAACGTGGTGCTGAAGACCGTGAGCGAGAGCGCCA | |
| | CCCAGCCCCCCACCAAGGTGTACAACAACGACCTGACCGC | |
| | CAGCCTGATGGTGGCCCTGGACAGCAACAACACCATGCCC | |
| | TTCACCCCCGCCGCCATGCGCAGCGAGACCCTGGGCTTCT | |
| | ACCCCTGGAAGCCCACCATCCCCACCCCCTGGCGCTACTAC | |
| | TTCCAGTGGGACCGCACCCTGATCCCCAGCCACACCGGCA | |
| | CCAGCGGCACCCCCACCAACATCTACCACGGCACCGACCC | |
| | CGACGACGTGCAGTTCTACACCATCGAGAACAGCGTGCCC | |
| | GTGCACCTGCTGCGCACCGGCGACGAGTTCGCCACCGGCA | |
| | CCTTCTTCTTCGACTGCAAGCCCTGCCGCCTGACCCACACC | |
| | TGGCAGACCAACCGCGCCCTGGGCCTGCCCCCCTTCCTGA | |
| | ACAGCCTGCCCCAGAGCGAGGGCGCCACCAACTTCGGCG | |
| | ACATCGGCGTGCAGCAGGACAAGCGCCGCGGCGTGACCC | |
| | AGATGGGCAACACCAACTACATCACCGAGGCCACCATCAT | |
| | GCGCCCCGCCGAGGTGGGCTACAGCGCCCCCTACTACAGC | |
| | TTCGAGGCCAGCACCCAGGGCCCCTTCAAGACCCCCATCG | |
| | CCGCCGGCCGCGGCGGCGCCCAGACCTACGAGAACCAGG | |
| | CCGCCGACGGCGACCCCCGCTACGCCTTCGGCCGCCAGCA | |
| | CGGCCAGAAGACCACCACCACCGGCGAGACCCCCGAGCG | |
| | CTTCACCTACATCGCCCACCAGGACACCGGCCGCTACCCC | |
| | GAGGGCGACTGGATCCAGAACATCAACTTCAACCTGCCCG | |
| | TGACCAACGACAACGTGCTGCTGCCCACCGACCCCATCGG | |
| | CGGCAAGACCGGCATCAACTACACCAACATCTTCAACACC | |
| | TACGGCCCCCTGACCGCCCTGAACAACGTGCCCCCCGTGT | |
| | ACCCCAACGGCCAGATCTGGGACAAGGAGTTCGACACCG | |
| | ACCTGAAGCCCCGCCTGCACGTGAACGCCCCCTTCGTGTG | |
| | CCAGAACAACTGCCCCGGCCAGCTGTTCGTGAAGGTGGCC | |
| | CCCAACCTGACCAACGAGTACGACCCCGACGCCAGCGCCA | |
| | ACATGAGCCGCATCGTGACCTACAGCGACTTCTGGTGGAA | |
| | GGGCAAGCTGGTGTTCAAGGCCAAGCTGCGCGCCAGCCA | |
| | CACCTGGAACCCCATCCAGCAGATGAGCATCAACGTGGAC | |
| | AACCAGTTCAACTACGTGCCCAGCAACATCGGCGGCATGA | |
| | AGATCGTGTACGAGAAGAGCCAGCTGGCCCCCCGCAAGCT | |
| | GTACTAATAACTCGAGCATGCATCTAGAGGTACATCTAGATA | |
| | GAGCTCGCTGATCAGCCTCGACTGTGCCTTCTAGTTGCCAG | |
| | CCATCTGTTGTTTGCCCCTCCCCCGTGCCTTCCTTGACCCT | |
| | GGAAGGTGCCACTCCCACTGTCCTTTCCTAATAAAATGAGG | |
| | AAATTGCATCGCATTGTCTGAGTAGGTGTCATTCTATTCTGG | |
| | GGGGTGGGGTGGGGCAGGACAGCAAGGGGGAGGATTGGG | |
| | AAGACAATAGCAGGCATGCTGGGGA | |
| Exemplary | GACATTGATTATTGACTAGTTATTAATAGTAATCAATTACGG | SEQ ID |
| CPV | GGTCATTAGTTCATAGCCCATATATGGAGTTCCGCGTTACAT | NO: 156 |
| Construct | AACTTACGGTAAATGGCCCGCCTGGCTGACCGCCCAACGA | |
| comprising | CCCCCGCCCATTGACGTCAATAATGACGTATGTTCCCATAGT | |
| a proto- | AACGCCAATAGGGACTTTCCATTGACGTCAATGGGTGGAG | |
| parvovirus | TATTTACGGTAAACTGCCCACTTGGCAGTACATCAAGTGTA | |
| variant | TCATATGCCAAGTACGCCCCCTATTGACGTCAATGACGGTA | |
| VP2 capsid | AATGGCCCGCCTGGCATTATGCCCAGTACATGACCTTATGG | |
| coding | GACTTTCCTACTTGGCAGTACATCTACGTATTAGTCATCGCT | |
| sequence | ATTACCATGGTGATGCGGTTTTGGCAGTACATCAATGGGCG | |
| CPV_VP2 | TGGATAGCGGTTTGACTCACGGGGATTTCCAAGTCTCCACC | |
| | CCATTGACGTCAATGGGAGTTTGTTTTGGCACCAAAATCAA | |
| | CGGGACTTTCCAAAATGTCGTAACAACTCCGCCCCATTGAC | |
| | GCAAATGGGCGGTAGGCGTGTACGGTGGGAGGTCTATATAA | |
| | GCAGAGCTCTCTGGCTAACTAGAGAACCCACTGCTTACTG | |
| | GCTTATCGAAATTAATACGACTCACTATAGGGAGACCCAAG | |
| | CTTGGTACCGGACTCTAGAGGATCCGGTACTCGAGGAACT | |
| | GAAAAACCAGAAAGTTAACTGGTAAGTTTAGTCTTTTTGTC | |
| | TTTTATTTCAGGTCCCGGATCCGGTGGTGGTGCAAATCAAA | |
| | GAACTGCTCCTCAGTGGATGTTGCCTTTACTTCTAGGCCTG | |
| | TACGGAAGTGTTACTTCTGCTCTAAAAGCTTGATTAATTAA | |
| | GGCCGCCACCATGAGCGACGGCGCCGTGCAGCCCGACGG | |
| | CGGCCAGCCCGCCGTGCGCAACGAGCGCGCCACCGGCAG | |
| | CGGCAACGGCAGCGGCGGCGGCGGCGGCGGCGGCAGCGG | |
| | CGGCGTGGGCATCAGCACCGGCACCTTCAACAACCAGACC | |
| | GAGTTCAAGTTCCTGGAGAACGGCTGGGTGGAGATCACCG | |
| | CCAACAGCAGCCGCCTGGTGCACCTGAACATGCCCGAGAG | |
| | CGAGAACTACCGCCGCGTGGTGGTGAACAACATGGACAAG | |
| | ACCGCCGTGAACGGCAACATGGCCCTGGACGACATCCACG | |
| | CCCAGATCGTGACCCCCTGGAGCCTGGTGGACGCCAACGC | |
| | CTGGGGCGTGTGGTTCAACCCCGGCGACTGGCAGCTGATC | |
| | GTGAACACCATGAGCGAGCTGCACCTGGTGAGCTTCGAGC | |
| | AGGAGATCTTCAACGTGGTGCTGAAGACCGTGAGCGAGA | |
| | GCGCCACCCAGCCCCCCACCAAGGTGTACAACAACGACCT | |
| | GACCGCCAGCCTGATGGTGGCCCTGGACAGCAACAACACC | |
| | ATGCCCTTCACCCCCGCCGCCATGCGCAGCGAGACCCTGG | |
| | GCTTCTACCCCTGGAAGCCCACCATCCCCACCCCCTGGCGC | |
| | TACTACTTCCAGTGGGACCGCACCCTGATCCCCAGCCACAC | |
| | CGGCACCAGCGGCACCCCCACCAACATCTACCACGGCACC | |
| | GACCCCGACGACGTGCAGTTCTACACCATCGAGAACAGCG | |
| | TGCCCGTGCACCTGCTGCGCACCGGCGACGAGTTCGCCAC | |
| | CGGCACCTTCTTCTTCGACTGCAAGCCCTGCCGCCTGACCC | |
| | ACACCTGGCAGACCAACCGCGCCCTGGGCCTGCCCCCCTT | |
| | CCTGAACAGCCTGCCCCAGAGCGAGGGCGCCACCAACTTC | |
| | GGCGACATCGGCGTGCAGCAGGACAAGCGCCGCGGCGTG | |
| | ACCCAGATGGGCAACACCAACTACATCACCGAGGCCACCA | |
| | TCATGCGCCCCGCCGAGGTGGGCTACAGCGCCCCCTACTAC | |
| | AGCTTCGAGGCCAGCACCCAGGGCCCCTTCAAGACCCCCA | |
| | TCGCCGCCGGCCGCGGCGGCGCCCAGACCTACGAGAACCA | |
| | GGCCGCCGACGGCGACCCCCGCTACGCCTTCGGCCGCCAG | |
| | CACGGCCAGAAGACCACCACCACCGGCGAGACCCCCGAG | |
| | CGCTTCACCTACATCGCCCACCAGGACACCGGCCGCTACC | |
| | CCGAGGGCGACTGGATCCAGAACATCAACTTCAACCTGCC | |
| | CGTGACCAACGACAACGTGCTGCTGCCCACCGACCCCATC | |
| | GGCGGCAAGACCGGCATCAACTACACCAACATCTTCAACA | |
| | CCTACGGCCCCCTGACCGCCCTGAACAACGTGCCCCCCGT | |
| | GTACCCCAACGGCCAGATCTGGGACAAGGAGTTCGACACC | |
| | GACCTGAAGCCCCGCCTGCACGTGAACGCCCCCTTCGTGT | |
| | GCCAGAACAACTGCCCCGGCCAGCTGTTCGTGAAGGTGGC | |
| | CCCCAACCTGACCAACGAGTACGACCCCGACGCCAGCGCC | |
| | AACATGAGCCGCATCGTGACCTACAGCGACTTCTGGTGGA | |
| | AGGGCAAGCTGGTGTTCAAGGCCAAGCTGCGCGCCAGCC | |
| | ACACCTGGAACCCCATCCAGCAGATGAGCATCAACGTGGA | |
| | CAACCAGTTCAACTACGTGCCCAGCAACATCGGCGGCATG | |
| | AAGATCGTGTACGAGAAGAGCCAGCTGGCCCCCCGCAAGC | |
| | TGTACTAATGACTCGAGCATGCATCTAGAGGTACATCTAGAT | |
| | AGAGCTCGCTGATCAGCCTCGACTGTGCCTTCTAGTTGCCA | |
| | GCCATCTGTTGTTTGCCCCTCCCCCGTGCCTTCCTTGACCC | |
| | TGGAAGGTGCCACTCCCACTGTCCTTTCCTAATAAAATGAG | |
| | GAAATTGCATCGCATTGTCTGAGTAGGTGTCATTCTATTCTG | |
| | GGGGGTGGGGTGGGGCAGGACAGCAAGGGGGAGGATTGG | |
| | GAAGACAATAGCAGGCATGCTGGGGA | |

d. Exemplary Heterologous Nucleic Acid Constructs

In some embodiments, constructs of the disclosure may comprise (i) a transgene or a portion thereof and a transgene promoter sequence, and (ii) 5′ and 3′ AAV inverted terminal repeats (ITRs). In some embodiments, a construct may be packaged within a protoparvovirus variant VP1 capsid polypeptide to produce a virion. In some embodiments, a virion is delivered to a selected target cell. In some embodiments, a transgene is a nucleic acid sequence, heterologous to a construct sequence, which encodes a polypeptide, protein, functional RNA molecule (e.g., miRNA, miRNA inhibitor) or other gene product, of interest. A nucleic acid transgene coding sequence is operatively linked to regulatory component(s) in a manner which permits transgene transcription, translation, and/or expression in a cell of a target tissue.

Constructs as described in the present disclosure may include one or more additional elements as described herein (e.g., regulatory elements e.g., one or more of a promoter, a poly A sequence, and an IRES).

In some embodiments, constructs of the present disclosure may be at least 3 Kb, at least 3.5 Kb, at least 4.0 Kb, at least 4.1 Kb, at least 4.2 Kb, at least 4.3 Kb, at least 4.4 Kb, at least 4.5 Kb, at least 4.6 Kb, at least 4.7 Kb, at least 4.8 Kb, at least 4.9 Kb, at least 5.0 Kb, at least 5.1 Kb, at least 5.2 Kb, at least 5.3 Kb, at least 5.4 Kb, at least 5.5 Kb, at least 5.6 Kb, at least 5.7 Kb, at least 5.8 Kb, at least 5.9 Kb, at least 6.0 Kb, at least 6.1 Kb, at least 6.2 Kb, at least 6.3 Kb, at least 6.4 Kb, at least 6.5 Kb.

Methods for obtaining constructs are known in the art. For example, to produce protoparvovirus constructs, methods typically involve culturing a host cell which comprises a VP1 capsid coding sequence encoding a protoparvovirus capsid polypeptide or fragment thereof; a construct comprising an AAV inverted terminal repeats (ITRs) and a transgene; a functional capsid rep gene; a functional ITR rep gene; and/or sufficient helper functions to permit packaging of the construct into a protoparvovirus capsid polypeptide.

In some embodiments, components to be cultured in a host cell to package a construct in a protoparvovirus VP1 capsid may be provided to the host cell in trans. Alternatively, one or more components (e.g., a construct, rep sequences, cap sequences, and/or helper functions) may be provided by a stable host cell that has been engineered to contain one or more such components using methods known to those of skill in the art. In some embodiments, such a stable host cell contains such component(s) under control of an inducible promoter. In some embodiments, such component(s) may be under control of a constitutive promoter. In some embodiments, a selected stable host cell may contain selected component(s) under control of a constitutive promoter and other selected component(s) under control of one or more inducible promoters. For example, a stable host cell may be generated that is derived from HEK293T cells (which contain El helper functions under the control of a constitutive promoter), but that contain rep and/or cap proteins under control of inducible promoters. Other stable host cells may be generated by one of skill in the art using routine methods.

A construct, rep sequences, cap sequences, and helper functions required for producing a protoparvovirus VP1 polypeptide of the disclosure may be delivered to a packaging host cell using any appropriate genetic element (e.g., construct). A selected genetic element may be delivered by any suitable method known in the art, e.g., to those with skill in nucleic acid manipulation and include genetic engineering, recombinant engineering, and synthetic techniques (see, e.g., Sambrook et al., Molecular Cloning: A Laboratory Manual, Cold Spring Harbor Press, Cold Spring Harbor, N.Y., which is incorporated in its entirety herein by reference). Similarly, methods of generating protoparvovirus virions are well known and any suitable method can be used with the present disclosure (see, e.g., K. Fisher et al., J. Virol., 70:520-532 (1993) and U.S. Pat. No. 5,478,745, each of which is incorporated in its entirety herein by reference).

i. Inverted Terminal Repeat Sequences (ITRs)

Sequences of a construct described herein may comprise a cis-acting 5′ and 3′ inverted terminal repeat sequences (ITRs) (See, e.g., B. J. Carter, in “Handbook of Parvoviruses,” ed., P. Tijsser, CRC Press, pp. 155 168 (1990), which is incorporated in its entirety herein by reference). In some embodiments, ITR sequences are about 145 nt in length. For example, wild type AAV2 ITRs are generally about 145 nt in length. Preferably, substantially the entire sequences encoding ITRs are used in a given molecule, although some degree of minor modification of these sequences is permissible. Ability to modify ITR sequences is within the skill of the art. (See, e.g., texts such as Sambrook et al. “Molecular Cloning. A Laboratory Manual,” 2d ed., Cold Spring Harbor Laboratory, New York (1989); and K. Fisher et al., J Virol., 70:520 532 (1996), each of which is incorporated in its entirety herein by reference). An example of such a molecule employed in the present disclosure is a “cis-acting” construct comprising a sequence encoding a transgene product, in which such a sequence and its associated regulatory elements are flanked by 5′ or “left” and 3′ or “right” AAV ITR sequences. 5′ and left designations refer to a position of an ITR sequence relative to an entire construct, read left to right, in a sense direction. For example, in some embodiments, a 5′ or left ITR is an ITR that is closest to a promoter (as opposed to a polyadenylation sequence) for a given construct, when a construct is depicted in a sense orientation, linearly. 3′ and right designations refer to a position of an ITR sequence relative to an entire construct, read left to right, in a sense direction. For example, in some embodiments, a 3′ or right ITR is an ITR that is closest to a polyadenylation sequence (as opposed to a promoter sequence) for a given construct, when a construct is depicted in a sense orientation, linearly. ITRs as provided herein are depicted in 5′ to 3′ order in accordance with a sense strand. Accordingly, one of skill in the art will appreciate that a 5′ or “left” orientation ITR can also be depicted as a 3′ or “right” ITR when converting from sense to antisense direction. Further, it is well within the ability of one of skill in the art to transform a given sense ITR sequence (e.g., a 5′/left AAV ITR) into an antisense sequence (e.g., 3′/right ITR sequence). Accordingly, based upon known AAV ITRs one of skill in the art would understand, in looking at sequences disclosed herein, whether an ITR was in a sense or antisense orientation and whether it would go on a “left” or “right” side of a construct, whether or not it is explicitly labeled as such. One of ordinary skill in the art would understand how to modify a given ITR sequence for use as either a 5′/left or 3′/right ITR, or an antisense version thereof.

ITR sequences may be obtained from any known virus. In some embodiments, an ITR is or comprises 145 nucleotides. In some embodiments an ITR is a wild-type AAV2 ITR. In some embodiments an ITR is derived from a wild-type AAV2 ITR and includes one or more modifications, e.g., truncations, deletions, substitutions or insertions as is known in the art. In some embodiments, an ITR comprises fewer than 145 nucleotides, e.g., 119, 127, 130, 134 or 141 nucleotides. For example, in some embodiments, an ITR comprises 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143 144, or 145 nucleotides.

In some embodiments, an ITR comprises (a) a dependoparvovirus ITR (b) an AAV ITR, optionally an AAV2 ITR, (c) a bocaparvovirus ITR, (d) a protoparvovirus ITR, (e) a tetraparvovirus ITR, or (f) an erythroparvovirus ITR. In certain embodiments, the ITR is a terminal palindrome with Rep binding elements and terminal resolution site (trs) that is structurally similar to the wild-type ITR. The ITR, in some embodiments, is from AAV1, 2, 3, etc. In certain embodiments, the ITR has the AAV2 RBE and trs. In some embodiments, the ITR is a chimera of different AAVs. In some embodiments, the ITR and the Rep protein are from AAV5. In some embodiments, the ITR is synthetic and is comprised of RBE motifs and terminal resolution site (trs) GGTTGG, AGTTGG, AGTTGA, RRTTRR. The typical T-shaped structure of the terminal palindrome consisting of the B/B′ and C/C′ stems may also be synthetically modified with substitutions and insertions that maintain the overall secondary structure based on folding prediction (available at URL (http) of unafold.rna.albany.edu/?q=mfold/DNA-Folding-Form). The stability of the ITR secondary structure is designated by the Gibbs free energy, delta G, with lower values, i.e., more negative, indicating greater stability. The full-length, 145 nt ITR has a computed ΔG=−69.91 kcal/mol. The B and C stems: GCCCGGGCAAAGCCCGGGCGTCGGGCGACCTTTGGTCGCCCG (SEQ ID NO: 144) have ΔG=−22.44 kcal/mol. Substitutions and insertions that result in a structure with ΔG=−15 kcal/mol to-30 kcal/mol are functionally equivalent and not distinct from the wild-type dependoparvovirus ITRs.

Any combination of ITRs and capsid polypeptides may be used in constructs of the present disclosure, for example, wild-type or variant AAV2 ITRs and AAV6 capsid, etc.

ii. Transgene

Among other things, the present disclosure provides that a virion described herein comprises a heterologous nucleic acid comprising a transgene. In some embodiments, a transgene encodes a receptor, toxin, a hormone, an enzyme, a marker protein encoded by a marker gene (see above), or a cell surface protein or a therapeutic protein, peptide or antibody or fragment thereof. In some embodiments, a transgene for use in construct compositions as disclosed herein encodes any polypeptide of which expression in the cell is desired, including, but not limited to antibodies, antigens, enzymes, receptors (cell surface or nuclear), hormones, lymphokines, cytokines, reporter polypeptides, growth factors, and functional fragments of any of the above.

In some embodiments, a transgene for use in a virion as disclosed herein encodes a polypeptide that is lacking or non-functional in the subject having a disease, including but not limited to any diseases described herein. In some embodiments, a disease is a genetic disease.

In some aspects, a transgene as described herein encodes a nucleic acid for use in methods of preventing or treating one or more genetic deficiencies or dysfunctions in a mammal, such as for example, a polypeptide deficiency or polypeptide excess in a mammal, and particularly for preventing, treating or reducing severity or extent of deficiency in a human manifesting one or more of disorders linked to a deficiency in such polypeptides in cells and tissues. In some embodiments, methods described herein involve administration of a transgene that encodes one or more therapeutic peptides, polypeptides, siRNAs, microRNAs, antisense nucleotides, etc. packaged in a virion described herein, preferably in a pharmaceutically acceptable composition, to a subject in an amount and for a period of time sufficient to prevent or treat a deficiency or disorder in a subject suffering from such a disorder.

Thus, in some embodiments, nucleic acids of interest for use in construct compositions as disclosed herein can encode one or more peptides, polypeptides, or proteins, which are useful for the treatment or prevention of a disease in a mammalian subject.

Exemplary nucleic acids of interest for use in compositions and methods as disclosed herein include but not limited to: BDNF, CNTF, CSF, EGF, FGF, G-SCF, GM-CSF, gonadotropin, IFN, IFG-1, M-CSF, NGF, PDGF, PEDF, TGF, VEGF, TGF-B2, TNF, prolactin, somatotropin, XIAP1, IL-1, IL-2, IL-3, IL-4, IL-5, IL-6, IL-7, IL-8, IL-9, IL-10, IL-10(187A), viral IL-10, IL-11, IL-12, IL-13, IL-14, IL-15, IL-16, IL-17, IL-18, VEGF, FGF, SDF-1, connexin 40, connexin 43, SCN4a, HIFia, SERCa2a, ADCYl, and ADCY6.

In some embodiments, a nucleic acid may comprise a coding sequence or a fragment thereof selected from the group consisting of a mammalian β globin gene (e.g., HBA1, HBA2, HBB, HBG1, HBG2, HBD, HBE1, and/or HBZ), alpha-hemoglobin stabilizing protein (AHSP), a B-cell lymphoma/leukemia 11A (BCL11A) gene, a Kruppel-like factor 1 (KLF1) gene, a CCR5 gene, a CXCR4 gene, a PPP1R12C (AAVS1) gene, an hypoxanthine phosphoribosyltransferase (HPRT) gene, an albumin gene, a Factor VIII gene, a Factor IX gene, a Leucine-rich repeat kinase 2 (LRRK2) gene, a Huntingtin (HTT) gene, a rhodopsin (RHO) gene, a Cystic Fibrosis Transmembrane Conductance Regulator (CFTR) gene, F8 or a fragment thereof (e.g., fragment encoding B-domain deleted polypeptide (e.g., VIII SQ, p-VIII)), a surfactant protein B gene (SFTPB), a T-cell receptor alpha (TRAC) gene, a T-cell receptor beta (TRBC) gene, a programmed cell death 1 (PD1) gene, a Cytotoxic T-Lymphocyte Antigen 4 (CTLA-4) gene, an human leukocyte antigen (HLA) A gene, an HLA B gene, an HLA C gene, an HLA-DPA gene, an HLA-DQ gene, an HLA-DRA gene, a LMP7 gene, a Transporter associated with Antigen Processing (TAP) 1 gene, a TAP2 gene, a tapasin gene (TAPBP), a class II major histocompatibility complex transactivator (CUT A) gene, a dystrophin gene (DMD), a glucocorticoid receptor gene (GR), an IL2RG gene, an RFX5 gene, a FAD2 gene, a FAD3 gene, a ZP15 gene, a KASII gene, a MDH gene, and/or an EPSPS gene.

In some embodiments, a transgene for use in a virion disclosed herein can be used to restore expression of genes that are reduced in expression, silenced, or otherwise dysfunctional in a subject. Similarly, in some embodiments, a transgene for use in a virion disclosed herein can also be used to knockdown expression of genes that are aberrantly expressed in a subject.

In some embodiments, a dysfunctional gene is a tumor suppressor that has been silenced in a subject having cancer. In some embodiments, a dysfunctional gene is an oncogene that is aberrantly expressed in a subject having a cancer. Exemplary genes associated with cancer (oncogenes and tumor suppressors) include but not limited to: AARS, ABCB 1, ABCC4, ABI2, ABL1, ABL2, ACK1, ACP2, ACY1, ADSL, AK1, AKR1C2, AKT1, ALB, ANPEP, ANXAS, ANXA7, AP2Ml, APC, ARHGAPS, ARHGEFS, ARID4A, ASNS, ATF4, ATM, ATPSB, ATPSO, AXL, BARDI, BAX, BCL2, BHLHB2, BLMH, BRAF, BRCA1, BRCA2, BTK, CANX, CAP1, CAPNI, CAPNS1, CAVI, CBFB, CBLB, CCL2, CCND1, CCND2, CCND3, CCNE1, CCTS, CCYR61, CD24, CD44, CD59, CDC20, CDC25, CDC25A, CDC25B, CDC2LS, CDK10, CDK4, CDK5, CDK9, CDKL1, CDKNIA, CDKNIB, CDKNIC, CDKN2A, CDKN2B, CDKN2D, CEBPG, CENPC1, CGRRF1, CHAFIA, CIB1, CKMT1, CLK1, CLK2, CLK3, CLNSIA, CLTC, COLIA1, COL6A3, COX6C, COX7A2, CRAT, CRHR1, CSF1R, CSK, CSNK1G2, CTNNA1, CTNNB1, CTPS, CTSC, CTSD, CULI, CYR61, DCC, DCN, DDX10, DEK, DHCR7, DHRS2, DHX8, DLG3, DVL1, DVL3, E2F1, E2F3, E2F5, EGFR, EGR1, EIF5, EPHA2, ERBB2, ERBB3, ERBB4, ERCC3, ETVI, ETV3, ETV6, F2R, FASTK, FBN1, FBN2, FES, FGFR1, FGR, FKBP8, FN1, FOS, FOSL1, FOSL2, FOXG1A, FOXO1A, FRAP1, FRZB, FTL, FZD2, FZDS, FZD9, G22P1, GAS6, GCNSL2, GDF1S, GNA13, GNAS, GNB2, GNB2L1, GPR39, GRB2, GSK3A, GSPT1, GTF21, HDAC1, HDGF, HMMR, HPRT1, HRB, HSPA4, HSPAS, HSPA8, HSPB1, HSPH1, HYAL1, HYOU1, ICAM1, ID1, ID2, IDUA, IER3, IFITM1, IGF1R, IGF2R, IGFBP3, IGFBP4, IGFBPS, IL1B, ILK, ING1, IRF3, ITGA3, ITGA6, ITGB4, JAK1, JARID1A, JUN, JUNB, JUND, K-ALPHA-1, KIT, KITLG, KLK10, KPNA2, KRAS2, KRT18, KRT2A, KRT9, LAMB1, LAMP2, LCK, LCN2, LEP, LITAF, LRPAP1, LTF, LYN, LZTR1, MADH1, MAP2K2, MAP3K8, MAPK12, MAPK13, MAPKAPK3, MAPRE1, MARS, MAS1, MCC, MCM2, MCM4, MDM2, MDM4, MET, MGST1, MICB, MLLT3, MME, MMP1, MMP14, MMP17, MMP2, MNDA, MSH2, MSH6, MT3, MYB, MYBL1, MYBL2, MYC, MYCL1, MYCN, MYD88, MYL9, MYLK, NEO1, NF1, NF2, NFKB I, NFKB2, NFSF7, NID, NINJ1, NMBR, NME1, NME2, NME3, NOTCH 1, NOTCH2, NOTCH4, NPM1, NQO1, NRID1, NR2Fl, NR2F6, NRAS, NRG1, NSEP1, OSM, PA2G4, PABPC1, PCNA, PCTK1, PCTK2, PCTK3, PDGFA, PDGFB, PDGFRA, PDPK1, PEA15, PFDN4, PFDN5, PGAM1, PHB, PIK3CA, PIK3CB, PIK3CG, PIM1, PKM2, PKMYT1, PLK2, PPARD, PPARG, PPIH, PPP1CA, PPP2RSA, PRDX2, PRDX4, PRKAR1A, PRKCBP1, PRNP, PRSS15, PSMA1, PTCH, PTEN, PTGS1, PTMA, PTN, PTPRN, RABSA, RAC1, RADSO, RAF1, RALBP1, RAP1A, RARA, RARB, RASGRF1, RB1, RBBP4, RBL2, REA, REL, RELA, RELB, RET, RFC2, RGS19, RHOA, RHOB, RHOC, RHOD, RIPK1, RPN2, RPS6 KB 1, RRM1, SARS, SELENBP1, SEMA3C, SEMA4D, SEPP1, SERPINH1, SFN, SFPQ, SFRS7, SHB, SHH, SIAH2, SIVA, SIVA TP53, SKI, SKIL, SLC16A1, SLC1A4, SLC20Al, SMO, SMPD1, SNAI2, SND1, SNRPB2, SOCS1, SOCS3, SOD1, SORT1, SPINT2, SPRY2, SRC, SRPX, STAT1, STAT2, STAT3, STAT5B, STC1, TAF1, TBL3, TBRG4, TCF1, TCF7L2, TFAP2C, TFDP1, TFDP2, TGFA, TGFB1, TGFBR1, TGFBR2, TGFBR3, THBS1, TIE, TIMP1, TIMP3, TJP1, TK1, TLE1, TNF, TNFRSF10A, TNFRSF10B, TNFRSF1A, TNFRSF1B, TNFRSF6, TNFSF7, TNK1, TOB1, TP53, TP53BP2, TP5313, TP73, TPBG, TPTI, TRADD, TRAM1, TRRAP, TSG101, TUFM, TXNRD1, TYR03, UBC, UBE2L6, UCHL1, USP7, VDAC1, VEGF, VHL, VIL2, WEE1, WNT1, WNT2, WNT2B, WNT3, WNTSA, WT1, XRCC 1, YES 1, YWHAB, YWHAZ, ZAP70, and ZNF9.

In some embodiments, a dysfunctional gene is HBB. In some embodiments, an HBB comprises at a nonsense, frameshift, or splicing mutation that reduces or eliminates β-globin production. In some embodiments, HBB comprises a mutation in a promoter region or polyadenylation signal of HBB. In some embodiments, an HBB mutation is at least one of c. 17A>T, c.-1360G, c.92+1G>A, c.92+6T>C, c.93-21G>A, c.1180T, c.316-1060G, c.25_26delAA, c.27_28insG, c.92+5G>C, c. 1180T, c. 135delC, c.315+1G>A, c.-78A>G, c.52A>T, c.59A>G, c.92+5G>C, c. 124_127delTTCT, c.316-1970T, c.-78A>G, c.52A>T, c. 124 127delTTCT, c.316-197C>T, C.-1380T, c.-79A>G, c.92+5G>C, c.75T>A, c.316-2A>G, and c.316-2A>C.

In certain embodiments, sickle cell disease is improved by gene therapy (e.g., stem cell gene therapy) that introduces an HBB variant that comprises at least one sequence variation comprising anti-sickling activity. In some embodiments, an HBB variant may be a double mutant (βAS2; T87Q and E22A). In some embodiments, an HBB variant may be a triple-mutant β-globin variant (βAS3; T87Q, E22A, and G16D). A modification at β16, glycine to aspartic acid, serves a competitive advantage over sickle globin (βS, HbS) for binding to a chain. A modification at β22, glutamic acid to alanine, partially enhances axial interaction with a20 histidine. These modifications result in anti-sickling properties greater than those of the single T87Q-modified variant and comparable to fetal globin. In a SCD murine model, transplantation of bone marrow stem cells transduced with SIN lentivirus carrying βAS3 reversed the red blood cell physiology and SCD clinical symptoms. Accordingly, this variant is being tested in a clinical trial (Identifier no: NCT02247843), Cytotherapy (2018) 20(7): 899-910.

In some embodiments, a dysfunctional gene is CFTR. In some embodiments, CFTR comprises a mutation selected from ΔF508, R553X, R74W, R668C, S977F, L997F, K1060T, A1067T, R1070Q, R1066H, T3381, R334W, G85E, A46D, I336K, H1054D, MIV, E92K, V520F, H1085R, R560T, L927P, R560S, N1303K, M1101K, L1077P, R1066M, R1066C, L1065P, Y569D, A561E, A559T, S492F, L467P, R347P, S341P, I507del, G1061R, G542X, W1282X, and 2184InsA.

In some embodiments, a transgene comprises a gene associated with a kidney disease.

In some embodiments, a transgene comprises a gene associated with Alport syndrome (e.g., Col4a3, Col4a4, Col4a5). In some embodiments, a transgene comprises or is Col4a3. In some embodiments, a transgene comprises or is Col4a4. In some embodiments, a transgene comprises or is Col4a5.

In some embodiments, a transgene comprises a gene associated with Fabry disease (e.g., GLA). In some embodiments, a transgene comprises or is GLA.

In some embodiments, a transgene comprises a gene associated with autosomal dominant polycystic kidney disease (PKD) (e.g., PKD1, PKD2). In some embodiments, a transgene comprises or is PKD. In some embodiments, a transgene comprises or is PKD1. In some embodiments, a transgene comprises or is PKD2.

In some embodiments, a transgene comprises a gene associated with congenital nephrotic syndrome (e.g., NPHS1 (Nephrin), NPHS2 (Podocin). In some embodiments, a transgene comprises or is NPHS1. In some embodiments, a transgene comprises or is NPHS2.

In some embodiments, a transgene comprises a gene associated with a cardiac disease (or heart disease).

In some embodiments, a transgene comprises a gene associated with hypertrophic cardiomyopathy (e.g., MYBPC3, JPH2, ALPK3). In some embodiments, a transgene comprises or is MYBPC3. In some embodiments, a transgene comprises or is JPH2. In some embodiments, a transgene comprises or is ALPK3.

In some embodiments, a transgene comprises a gene associated with dilated cardiomyopathy (e.g., RBM20). In some embodiments, a transgene comprises or is RBM20.

In some embodiments, a transgene comprises a gene associated with dilated cardiomyopathy (e.g., ALPK3, LMNA, BAG3). In some embodiments, a transgene comprises or is ALPK3. In some embodiments, a transgene comprises or is LMNA. In some embodiments, a transgene comprises or is BAG3.

In some embodiments, a transgene as defined herein encodes a small interfering nucleic acid (e.g., shRNAs, miRNAs) that inhibits the expression of a gene product associated with cancer (e.g., oncogenes) may be used to prevent or treat cancer. In some embodiments, a transgene as defined herein encodes a gene product associated with cancer (or a functional RNA that inhibits expression of a gene associated with cancer) for use, e.g., for research purposes, e.g., to study a cancer or to identify therapeutics that prevent or treat a cancer.

An ordinarily skilled artisan also appreciates that a nucleic acids of interest can comprise at least one sequence variation that result in conservative amino acid substitutions which may provide functionally equivalent variants, or homologs of a protein or polypeptide. Additionally contemplated in this disclosure is a transgene in a virion described herein, having a dominant negative mutation. For example, a transgene can encode a mutant protein that interacts with the same elements as a wild-type protein, and thereby blocks some aspects of a function of a wild-type protein.

In some embodiments, a transgene in a virion disclosed herein includes miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA.

A miRNA inhibits the function of the mRNAs it targets and, as a result, inhibits expression of the polypeptides encoded by the mRNAs. Thus, blocking (partially or totally) the activity of the miRNA (e.g., silencing the miRNA) can effectively induce, or restore, expression of a polypeptide whose expression is inhibited (de-repress the polypeptide). In some embodiments, de-repression of polypeptides encoded by mRNA targets of a miRNA is accomplished by inhibiting the miRNA activity in cells through any one of a variety of methods. For example, blocking activity of a miRNA can be accomplished by hybridization with a small interfering nucleic acid (e.g., antisense oligonucleotide, miRNA sponge, TuD RNA) that is complementary, or substantially complementary to, the miRNA, thereby blocking interaction of the miRNA with its target mRNA. As used herein, a small interfering nucleic acid that is substantially complementary to a miRNA is one that is capable of hybridizing with a miRNA, and blocking the miRNA's activity. In some embodiments, a small interfering nucleic acid that is substantially complementary to a miRNA is a small interfering nucleic acid that is complementary with the miRNA at all but 1, 2, 3, 4, 5, 6, 7, 8, 9, 10, 11, 12, 13, 14, 15, 16, 17, or 18 bases. In some embodiments, a small interfering nucleic acid sequence that is substantially complementary to a miRNA, is a small interfering nucleic acid sequence that is complementary with the miRNA at, at least, one base.

iii. Transgene Promoter Sequences

In some embodiments, a transgene promoter is an inducible promoter, a constitutive promoter, a mammalian cell promoter, a viral promoter, a chimeric promoter, an engineered promoter, a tissue-specific promoter, or any other type of promoter known in the art. In some embodiments, a promoter is a RNA polymerase II promoter, such as a mammalian RNA polymerase II promoter. In some embodiments, a promoter is a RNA polymerase III promoter, including, but not limited to, a HI promoter, a human U6 promoter, a mouse U6 promoter, or a swine U6 promoter.

In some embodiments, a transgene promoter can be a transgene promoter that, in its endogenous context, is associated with a gene in the CRISPR/Cas system. For example, in some embodiments, a promoter can be a Cas gene promoter. In some embodiments, a transgene promoter can be a Cas9 promoter.

A variety of transgene promoters is known in the art, any of which can be used herein. Non-limiting examples of transgene promoters that can be used herein include transgene promoters for: human elongation factor 1α-subunit (EF1a) (Liu et al. (2007) Exp. Mol. Med. 39(2): 170-175; Accession No. J04617.1; Gill et al., Gene Ther. 8(20): 1539-1546, 2001; Xu et al., Human Gene Ther. 12(5): 563-573, 2001; Xu et al., Gene Ther. 8:1323-1332; Ikeda et al., Gene Ther. 9:932-938, 2002; Gilham et al., J. Gene Med. 12(2): 129-136, 2010, each of which is incorporated in its entirety herein by reference), cytomegalovirus (Xu et al., Human Gene Ther. 12(5): 563-573, 2001; Xu et al., Gene Ther. 8:1323-1332; Gray et al., Human Gene Ther. 22:1143-1153, 2011, each of which is incorporated in its entirety herein by reference), human immediate-early cytomegalovirus (CMV) (U.S. Pat. No. 5,168,062, Liu et al. (2007) Exp. Mol. Med. 39(2): 170-175; Accession No. X17403.1 or KY490085.1, each of which is incorporated in its entirety herein by reference), human ubiquitin C (UBC) (Gill et al., Gene Ther. 8(20): 1539-1546, 2001; Qin et al., PLOS One 5(5): e10611, 2010, each of which is incorporated in its entirety herein by reference), mouse phosphoglycerate kinase 1, polyoma adenovirus, simian virus 40 (SV40), β-globin, β-actin, α-fetoprotein, γ-globin, β-interferon, γ-glutamyl transferase, mouse mammary tumor virus (MMTV), Rous sarcoma virus, rat insulin, glyceraldehyde-3-phosphate dehydrogenase, metallothionein II (MT II), amylase, cathepsin, MI muscarinic receptor, retroviral LTR (e.g., human T-cell leukemia virus HTLV, each of which is incorporated in its entirety herein by reference), AAV ITR, interleukin-2, collagenase, platelet-derived growth factor, adenovirus 5 E2, stromelysin, murine MX gene, glucose regulated proteins (GRP78 and GRP94), α-2-macroglobulin, vimentin, MHC class I gene H-2κ b, HSP70, proliferin, tumor necrosis factor, thyroid stimulating hormone α gene, immunoglobulin light chain, T-cell receptor, HLA DQα and DQβ, interleukin-2 receptor, MHC class II, MHC class II HLA-DRα, muscle creatine kinase, prealbumin (transthyretin), elastase I, albumin gene, c-fos, c-HA-ras, neural cell adhesion molecule (NCAM), H2B (TH2B) histone, rat growth hormone, human serum amyloid (SAA), troponin I (TN I), duchenne muscular dystrophy, human immunodeficiency virus, Gibbon Ape Leukemia Virus (GALV) promoters, promoter of HNRPA2β1-CBX1 (UCOE) (Powell and Gray (2015) Discov. Med. 19(102): 49-57; Antoniou et al., Human Gene Ther. 24(4): 363-374, 2013), β-glucuronidase (GUSB) (Husain et al., Gene Ther. 16:927-932, 2009), chicken β-actin (CBA) (Liu et al. (2007) Exp. Mol. Med. 39(2): 170-175; Stone et al. (2005) Mol. Ther. 11(6): 843-848; Klein et al., Exp. Neurol. 176(1): 66-74, 2002; Ohlfest et al., Blood 105:2691-2698, 2005; Gray et al., Human Gene Ther. 22:1143-1153, 2011, each of which is incorporated in its entirety herein by reference), a human β-actin promoter (HBA) (Accession No. Y00474.1), murine myosin VIIA (musMyo7) (Boeda et al. (2001) Hum. Mol. Genet. 10(15): 1581-1589; Accession No. AF384559.1, each of which is incorporated in its entirety herein by reference), human myosin VIIA (hsMyo7) (Boeda et al. (2001) Hum. Mol. Genet. 10(15): 1581-1589; Accession No. NG_009086.1, each of which is incorporated in its entirety herein by reference), murine poly(ADP-ribose) polymerase 2 (musPARP2) (Ame et al. (2001) J. Biol. Chem. 276(14): 11092-11099; Accession No. ΔF191547.1, each of which is incorporated in its entirety herein by reference), human poly(ADP-ribose) polymerase 2 (hsPARP2) (Ame et al. (2001) J. Biol. Chem. 276(14): 11092-11099; Accession No. X16612.1 or AF479321.1, each of which is incorporated in its entirety herein by reference), acetylcholine receptor epsilon-subunit (AChε) (Duclert et al. (1993) PNAS 90(7): 3043-3047; Accession No. S58221.1 or CR933736.12, each of which is incorporated in its entirety herein by reference), Rous sarcoma virus (RSV) (Liu et al. (2007) Exp. Mol. Med. 39(2): 170-175; Accession No. M77786.1, each of which is incorporated in its entirety herein by reference), (GFAP) (Liu et al. (2007) Exp. Mol. Med. 39(2): 170-175; Stone et al. (2005) Mol. Ther. 11(6): 843-848; Accession No. NG 008401.1 or M67446.1, each of which is incorporated in its entirety herein by reference), hAAT (Van Linthout et al., Human Gene Ther. 13(7): 829-840, 2002; Cunningham et al., Mol. Ther. 16(6): 1081-1088, 2008, each of which is incorporated in its entirety herein by reference), and a CBA hybrid (CBh) (Gray et al. (2011) Hum. Gen. Therapy 22:1143-1153; Accession No. KF926476.1 or KC152483.1, each of which is incorporated in its entirety herein by reference). Additional examples of promoters are known in the art. See, e.g., Lodish, Molecular Cell Biology, Freeman and Company, New York 2007. The contents of each of these references are incorporated by reference in its entirety.

In some embodiments, a promoter is a CMV immediate early promoter.

In some embodiments, a promoter is a CAG promoter or a CAG/CBA promoter.

The term “constitutive” transgene promoter refers to a nucleotide sequence that, when operably linked with a nucleic acid encoding a protein a nucleic acid.

Examples of constitutive transgene promoters include, without limitation, a retroviral Rous sarcoma virus (RSV) LTR promoter, a cytomegalovirus (CMV) promoter (see, e.g., Boshart et al. Cell 41:521-530, 1985, which is incorporated in its entirety herein by reference), an SV40 promoter, a dihydrofolate reductase promoter, a beta-actin promoter, a phosphoglycerol kinase (PGK) promoter, and an EF1-alpha promoter (Invitrogen).

In some embodiments, inducible transgene promoters allow regulation of gene expression and can be regulated by exogenously supplied compounds, environmental factors such as temperature, or presence of a specific physiological state, e.g., acute phase, a particular functional or biological state of a cell, e.g., a particular differentiation state of a cell, or in replicating cells only. Inducible promoters and inducible systems are available from a variety of commercial sources, including, without limitation, Invitrogen, Clontech, and Ariad. Additional examples of inducible promoters are known in the art.

Examples of inducible transgene promoters regulated by exogenously supplied compounds include a zinc-inducible sheep metallothionine (MT) promoter, a dexamethasone (Dex)-inducible mouse mammary tumor virus (MMTV) promoter, a T7 polymerase promoter system (WO 98/10088, which is incorporated in its entirety herein by reference); an ecdysone insect promoter (No et al. Proc. Natl. Acad. Sci. U.S.A. 93:3346-3351, 1996, which is incorporated in its entirety herein by reference), a tetracycline-repressible system (Gossen et al. Proc. Natl. Acad. Sci. U.S.A. 89:5547-5551, 1992, which is incorporated in its entirety herein by reference), a tetracycline-inducible system (Gossen et al. Science 268:1766-1769, 1995, see also Harvey et al. Curr. Opin. Chem. Biol. 2:512-518, 1998, each of which is incorporated in its entirety herein by reference), an RU486-inducible system (Wang et al. Nat. Biotech. 15:239-243, 1997; and Wang et al. Gene Ther. 4:432-441, 1997, each of which is incorporated in its entirety herein by reference), and a rapamycin-inducible system (Magari et al. J. Clin. Invest. 100:2865-2872, 1997, which is incorporated in its entirety herein by reference).

In some embodiments, regulatory sequences impart tissue-specific gene expression capabilities. In some cases, tissue-specific regulatory sequences bind tissue-specific transcription factors that induce transcription in a tissue-specific manner.

The term “tissue-specific” transgene promoter refers to a transgene promoter that is active only in certain specific cell types and/or tissues (e.g., transcription of a specific gene occurs only within cells expressing transcription regulatory and/or control proteins that bind to the tissue-specific promoter).

In some embodiments, provided constructs comprise a promoter sequence selected from a CAG, a CBA, a CMV, or a CB7 promoter. In some embodiments of therapeutic compositions described herein, a first or sole a construct further includes at least one promoter.

iv. Enhancers and 5′ Cap

In some instances, a construct can include a transgene promoter sequence and/or an enhancer sequence. In some embodiments, an enhancer is a nucleotide sequence that can increase a level of transcription of a nucleic acid encoding a polypeptide of interest (e.g., a transgene). In some embodiments, enhancer sequences (50-1500 base pairs in length) generally increase a level of transcription by providing additional binding sites for transcription-associated proteins (e.g., transcription factors). In some embodiments, an enhancer sequence is found within an intronic sequence. Unlike promoter sequences, enhancer sequences can act at much larger distance away from a transcription start site (e.g., as compared to a promoter). Non-limiting examples of enhancers include a RSV enhancer, a CMV enhancer, and a SV40 enhancer. An example of a CMV enhancer is described in, e.g., Boshart et al., Cell 41(2): 521-530, 1985, which is incorporated in its entirety herein by reference.

As described herein, a 5′ cap (also termed an RNA cap, an RNA 7-methylguanosine cap or an RNA m.sup.7G cap) is a modified guanine nucleotide that has been added to a “front” or 5′ end of a eukaryotic messenger RNA shortly after a start of transcription. In some embodiments, a 5′ cap consists of a terminal group which is linked to a first transcribed nucleotide. Its presence is critical for recognition by a ribosome and protection from RNases. Cap addition is coupled to transcription, and occurs co-transcriptionally, such that each influences the other. Shortly after start of transcription, a 5′ end of an mRNA being synthesized is bound by a cap-synthesizing complex associated with RNA polymerase. This enzymatic complex catalyzes a chemical reactions that are required for mRNA capping. Synthesis proceeds as a multi-step biochemical reaction. A capping moiety can be modified to modulate functionality of mRNA such as its stability or efficiency of translation.

e. Reporter Sequences or Elements

Any constructs provided herein can optionally include a sequence encoding a reporter protein (“a reporter sequence”). For example, in some embodiments, a reporter sequence may be a FLAG, an eGFP, an mScarlet, a luciferase or any variant thereof. In some embodiments, a reporter sequence is visibly detectable without intervention. In some embodiments, a reporter element may be detected using a combination of fluorescent, histochemical, and/or transcript or protein analyses. Non-limiting examples of reporter sequences are described herein. Additional examples of reporter sequences are known in the art. In some embodiments, reporter sequence can be used to verify tissue-specific targeting capabilities and tissue-specific promoter regulatory activity of any constructs described herein.

f. Additional Sequences

In some embodiments, constructs of the present disclosure may comprise a T2A element or sequence. In some embodiments, constructs of the present disclosure may include one or more cloning sites. In some such embodiments, cloning sites may not be fully removed prior to manufacturing for administration to a subject.

g. Genome Editing

In some embodiments, a genome editing system targets nucleotides within a specific target site.

i. RNA-Guided Micleases

RNA-guided nucleases according to the present disclosure include, but are not limited to, naturally-occurring Class 2 CRISPR nucleases such as Cas9, and Cpf1, as well as other nucleases derived or obtained therefrom. In functional terms, RNA-guided nucleases are defined as those nucleases that: (a) interact with (e.g., complex with) a gRNA; and (b) together with gRNA, associate with, and optionally cleave or modify, a target region of a DNA that includes (i) a sequence complementary to a targeting domain of a gRNA and, optionally, (ii) an additional sequence referred to as a “protospacer adjacent motif,” or “PAM,” which is described in greater detail herein.

Naturally occurring CRISPR systems are organized evolutionarily into two classes and five types (Makarova et al. Nat Rev Microbiol. 2011 June; 9(6): 467-477 (“Makarova”), which is incorporated in its entirety herein by reference), and while genome editing systems of the present disclosure may adapt components of any type or class of naturally occurring CRISPR system, embodiments presented herein are generally adapted from Class 2, and type II or V CRISPR systems. Class 2 systems, which encompass types II and V, are characterized by relatively large, multidomain CRISPR proteins (e.g., Cas9 or Cpf1) and one or more gRNAs (e.g., a crRNA and, optionally, a tracrRNA) that form ribonucleoprotein (RNP) complexes that associate with (i.e., target) and cleave specific loci complementary to a targeting (or spacer) sequence of a crRNA. Genome editing systems according to the present disclosure similarly target and edit cellular DNA sequences, but differ significantly from CRISPR systems occurring in nature. For example, unimolecular gRNAs described herein do not occur in nature, and both gRNAs and CRISPR nucleases according to this disclosure may incorporate any number of non-naturally occurring modifications.

As described herein, it should be noted that a genome editing systems of the present disclosure can be targeted to a single specific nucleotide sequence, or may be targeted to—and capable of editing in parallel—two or more specific nucleotide sequences through use of two or more gRNAs. In some embodiments, use of multiple gRNAs is referred to as “multiplexing.” As described herein, multiplexing can be employed, for example, to target multiple, unrelated target sequences of interest, or to form multiple SSBs or DSBs within a single target domain and, in some cases, to generate specific edits within such target domain. For example, International Patent Publication No. WO 2015/138510 by Maeder et al., which is incorporated in its entirety herein by reference; (“Maeder”) describes a genome editing system for correcting a point mutation (C.2991+1655A to G) in human CEP290 that results in t creation of a cryptic splice site, which in turn reduces or eliminates function of the gene. That genome editing system of Maeder utilizes two gRNAs targeted to sequences on either side of (i.e., flanking) the point mutation, and forms DSBs that flank the mutation. This, in turn, promotes deletion of the intervening sequence, including the mutation, thereby eliminating the cryptic splice site and restoring normal gene function.

As another example, WO 2016/073990 by Cotta-Ramusino, et al. (“Cotta-Ramusino”), which is incorporated in its entirety herein by reference. Cotta-Ramusino describes a genome editing system that utilizes two gRNAs in combination with a Cas9 nickase (a Cas9 that makes a single strand nick such as S. pyogenes D10A), an arrangement termed a “dual-nickase system.” The dual-nickase system of Cotta-Ramusino is configured to make two nicks on opposite strands of a sequence of interest that are offset by one or more nucleotides, which nicks combine to create a double strand break having an overhang (5′ in the case of Cotta-Ramusino, though 3′ overhangs are also possible). The overhang, in turn, can facilitate homology directed repair events in some circumstances. And, as another example, WO 2015/070083 by Palestrant et al., which is incorporated in its entirety herein by reference; (“Palestrant”) describes a gRNA targeted to a nucleotide sequence encoding Cas9 (referred to as a “governing RNA”), which can be included in a genome editing system comprising one or more additional gRNAs to permit transient expression of a Cas9 that might otherwise be constitutively expressed, for example in some virally transduced cells. These multiplexing applications are intended to be exemplary, rather than limiting, and the skilled artisan will appreciate that other applications of multiplexing are generally compatible with the genome editing systems described here.

Genome editing systems can, in some instances, form double strand breaks that are repaired by cellular DNA double-strand break mechanisms such as NHEJ or HDR. These mechanisms are described throughout the literature, for example by Davis & Maizels, PNAS, 111(10): E924-932, Mar. 11, 2014, which is incorporated in its entirety herein by reference (“Davis”) (describing Alt-HDR); Frit et al. DNA Repair 17(2014) 81-97, which is incorporated in its entirety herein by reference (“Frit”) (describing Alt-NHEJ); and Iyama and Wilson III, DNA Repair (Amst.) 2013-August; 12(8): 620-636, which is incorporated in its entirety herein by reference (“Iyama”) (describing canonical HDR and NHEJ pathways generally).

Where genome editing systems operate by forming DSBs, such systems optionally include one or more components that promote or facilitate a particular mode of double-strand break repair or a particular repair outcome. For instance, Cotta-Ramusino also describes genome editing systems in which a single stranded oligonucleotide “donor template” is added; a donor template is incorporated into a target region of cellular DNA that is cleaved by a genome editing system, and can result in a change in a target sequence.

In some embodiments, genome editing systems modify a target sequence, or modify expression of a gene in or near a target sequence, without causing single- or double-strand breaks. For example, a genome editing system may include a CRISPR protein fused to a functional domain that acts on DNA, thereby modifying a target sequence or its expression. As one example, a CRISPR protein can be connected to (e.g., fused to) a cytidine deaminase functional domain, and may operate by generating targeted C-to-A substitutions. Exemplary nuclease/deaminase fusions are described in Komor et al. Nature 533, 420-424(19 May 2016) (“Komor”), which is incorporated in its entirety herein by reference. In some embodiments, a genome editing system may utilize a cleavage-inactivated (i.e., a “dead”) nuclease, such as a dead Cas9 (dCas9), and may operate by forming stable complexes on one or more targeted regions of cellular DNA, thereby interfering with functions involving a targeted region(s) including, without limitation, mRNA transcription, chromatin remodeling, etc. In some embodiments, a genome editing system may be self-inactivating to improve a safety profile, as described by Li et al. “A Self-Deleting AAV-CRISPR System for In vivo Editing” Mol Ther Methods Clin Dev. 2019 Mar. 15; 12:111-122; published online (2018 Dec. 6), the contents of which are hereby incorporated by reference in its entirety.

As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Cas9 vs. Cpf1), species (e.g., S. pyogenes vs. S. aureus, etc.) or variation (e.g., full-length vs. truncated or split; naturally-occurring PAM specificity vs. engineered PAM specificity, etc.) of RNA-guided nuclease. In some embodiments, a CRISPR/Cas is derived from a type II CRISPR/Cas system. In some embodiments, a CRISPR/Cas system is derived from a Cas9 protein. A Cas9 protein can be from Streptococcus pyogenes, Streptococcus thermophilus, Staphylococcus aureus, Campylobacter jejuni, or other species.

Administering bacterial Cas9 in humans presents immunogenicity concerns. Therefore, it is important to develop a codon-optimized CRISPR system as described herein to reduce immunogenicity. In addition, some other limitations include a need to use a two construct system (instead of a single construct system such that is used in shRNA and miRNA protocols), and determination of off-target risk.

A PAM sequence takes its name from its sequential relationship to a “protospacer” sequence that is complementary to gRNA targeting domains (or “spacers”). Together with protospacer sequences, PAM sequences define target regions or sequences for specific RNA-guided nuclease/gRNA combinations.

Various RNA-guided nucleases may require different sequential relationships between PAMs and protospacers. In general, Cas9s recognize PAM sequences that are 3′ of a protospacer. Cpf1, on the other hand, generally recognizes PAM sequences that are 5′ of a protospacer.

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. And F. novicida Cpf1 recognizes a TTN PAM sequence. PAM sequences have been identified for a variety of RNA-guided nucleases, and a strategy for identifying novel PAM sequences has been described by Shmakov et al., 2015, Molecular Cell 60, 385-397, Nov. 5, 2015. It should also be noted that engineered RNA-guided nucleases can have PAM specificities that differ from\PAM specificities of reference molecules (for instance, in the case of an engineered RNA-guided nuclease, a reference molecule may be a naturally occurring variant from which an RNA-guided nuclease is derived, or a naturally occurring variant having the greatest amino acid sequence homology to an engineered RNA-guided nuclease).

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above) Ran & Hsu, et al., Cell 154(6), 1380-1389 Sep. 12, 2013 (“Ran”)), or that that do not cut at all.

The present application also recognizes that other types of CRISPR enzymes, such as Cas12a, can be used in accordance with embodiments described herein.

### CRISPR Fusion Proteins

As described herein, in some embodiments, a CRISPR nuclease is part of a fusion protein comprising one or more heterologous protein domains (e.g., about or more than about 1, 2, 3, 4, 5, 6, 7, 8, 9, 10, or more domains in addition to a CRISPR nuclease). A CRISPR nuclease fusion protein may comprise any additional protein sequence, and optionally a linker sequence between any two domains. Examples of protein domains that may be fused to a CRISPR nuclease include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR nuclease are described in US20110059502, incorporated herein by reference. In some embodiments, a tagged CRISPR nuclease is used to identify a location of a target sequence. In some embodiments, a CRISPR nuclease that is part of a fusion protein has been engineered to produce only SSBs as described herein. In some embodiments, a CRISPR nuclease that is part of a fusion protein has been engineered to not cut at all as described herein.

### CRISPR Variants

In general, RNA-guided nucleases comprise at least one RNA recognition and/or RNA binding domain. RNA recognition and/or RNA binding domains interact with a guiding RNA. CRISPR/Cas proteins can also comprise nuclease domains (i.e., DNase or RNase domains), DNA binding domains, helicase domains, RNAse domains, protein-protein interaction domains, dimerization domains, as well as other domains. RNA-guided nucleases can be modified to increase nucleic acid binding affinity and/or specificity, alter an enzymatic activity, and/or change another property of a protein. In some embodiments, a CRISPR/Cas-like protein of a fusion protein can be derived from a wild type Cas9 protein or fragment thereof. In some embodiments, a CRISPR/Cas can be derived from modified Cas9 protein. For example, an amino acid sequence of a Cas9 protein can be modified to alter one or more properties (e.g., nuclease activity, affinity, stability, and so forth) of a protein. Alternatively, domains of a Cas9 protein not involved in RNA-guided cleavage can be eliminated from a protein such that a modified Cas9 protein is smaller than a wild type Cas9 protein. In general, a Cas9 protein comprises at least two nuclease (i.e., DNase) domains. For example, a Cas9 protein can comprise a RuvC-like nuclease domain and a HNH-like nuclease domain. RuvC and HNH domains work together to cut single strands to make a double-stranded break in DNA (Jinek et al., 2012, Science, 337:816-821, which is incorporated in its entirety herein by reference).

In some embodiments, a Cas9-derived protein can be modified to contain only one functional nuclease domain (either a RuvC-like or a HNH-like nuclease domain). For example, a Cas9-derived protein can be modified such that one nuclease domain is deleted or mutated such that it is no longer functional (i.e., nuclease activity is absent). In some embodiments in which one nuclease domains is inactive, a Cas9-derived protein is able to introduce a nick into a double-stranded nucleic acid (such protein is termed a “nickase”), but not cleave double-stranded DNA. In any of the above-described embodiments, any or all of nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

One example of a CRISPR/Cas9 system used to inhibit gene expression, CRISPRi, is described in U.S. Publication No. US2014/0068797, which is incorporated herein by reference in its entirety. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a gRNA, a DNA recognition complex is generated that specifically interferes with transcriptional elongation, RNA polymerase binding, or transcription factor binding. This CRISPRi system efficiently represses expression of targeted genes.

ii. Guide RNAs (gRNAs)
gRNA Sequence Selection

A gRNA sequence may be specific for any gene, such as a gene that would affect (e.g., ameliorate, improve, attenuate, mitigate) a disease or disorder. In some embodiments, a gRNA sequence includes an RNA sequence, a DNA sequence, a combination thereof (a RNA-DNA combination sequence), or a sequence with synthetic nucleotides. A gRNA sequence can be a single molecule or a double molecule. In one embodiment, a gRNA sequence comprises a single guide RNA (sgRNA).

In some embodiments, a gRNA sequence is specific for a gene and targets that gene for Cas endonuclease-induced double strand breaks. A sequence of a gRNA may be within a loci of the gene. In one embodiment, a gRNA sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length. In some embodiments, a gRNA sequence is from about 18 to about 22 nucleotides in length.

As described herein, in some embodiments in the context of formation of a CRISPR complex, “target sequence” refers to a sequence to which a guide sequence is designed to have some complementarity, where hybridization between a target sequence and a guide sequence promotes formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, a target sequence may be within an organelle of a eukaryotic cell, for example, mitochondrion or nucleus. Typically, in the context of an endogenous CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) a target sequence. As with a target sequence, it is believed that complete complementarity is not needed, provided this is sufficient to be functional. In some embodiments, a tracr sequence has at least 50%, 60%, 70%, 80%, 90%, 95% or 99% of sequence complementarity along the length of a tracr mate sequence when optimally aligned.

gRNA Design

Methods for selection and validation of target sequences as well as off-target analyses have been described previously, e.g., in Mali; Hsu; Fu et al., 2014 Nat biotechnol 32(3): 279-84, Heigwer et al., 2014 Nat methods 11(2): 122-3; Bae et al. (2014) Bioinformatics 30(10): 1473-5; and Xiao A et al. (2014) Bioinformatics 30(8): 1180-1182, each of which is incorporated in its entirety herein by reference. As a non-limiting example, gRNA design may involve use of a software tool to optimize choice of potential target sequences corresponding to a user's target sequence, e.g., to minimize total off-target activity across a genome. While off-target activity is not limited to cleavage, cleavage efficiency at each off-target sequence can be predicted, e.g., using an experimentally-derived weighting scheme. These and other guide selection methods are described in detail in Maeder and Cotta-Ramusino.

For example, methods for selection and validation of target sequences as well as off-target analyses can be performed using cas-offinder (Bae S, Park J, Kim J-S. Cas-OFFinder: a fast and versatile algorithm that searches for potential off-target sites of Cas9 RNA-guided endonucleases. Bioinformatics. 2014; 30:1473-5, which is incorporated in its entirety herein by reference). Cas-offinder is a tool that can quickly identify all sequences in a genome that have up to a specified number of mismatches to a guide sequence.

As another example, methods for scoring how likely a given sequence is to be an off-target (e.g., once candidate target sequences are identified) can be performed. An exemplary score includes a Cutting Frequency Determination (CFD) score, as described by Doench J G, Fusi N, Sullender M, Hegde M, Vaimberg E W, Donovan K F, et al. Optimized sgRNA design to maximize activity and minimize off-target effects of CRISPR-Cas9. Nat Biotechnol. 2016; 34:184-91, which is incorporated in its entirety herein by reference.

gRNA Modifications

Certain exemplary modifications discussed in this section can be included at any position within a gRNA sequence including, without limitation at or near its 5′ end (e.g., within 1-10, 1-5, or 1-2 nucleotides of a 5′ end) and/or at or near its 3′ end (e.g., within 1-10, 1-5, or 1-2 nucleotides of a 3′ end). In some cases, modifications are positioned within functional motifs, such as a repeat-anti-repeat duplex of a Cas9 gRNA, a stem loop structure of a Cas9 or Cpf1 gRNA, and/or a targeting domain of a gRNA. Others types of modified nucleobases are described herein.

h. Knockdown

The present disclosure provides technologies (e.g., comprising compositions) that may, in some embodiments, reduce, suppress or otherwise decrease (“knock down”) expression of one or more gene products. For example, in some embodiments, technologies of the present disclosure may achieve knockdown of a gene product (e.g., a gene, mRNA, protein, etc.).

i. Inhibitory Nucleic Acid Molecules

RNA interference (RNAi) is a process of sequence-specific post-transcriptional gene silencing by which, e.g., double stranded RNA (dsRNA) homologous to a target locus can specifically inactivate gene function (Hammond et al., Nature Genet. 2001; 2:110-119; Sharp, Genes Dev. 1999; 13:139-141, the contents of each which are hereby incorporated by reference herein in its entirety). For example, positional location of shRNAs targeting intronic-3 XmiR, poly A-3 XmiR, or both intronic-3 XmiR and PolyA-3XmiR reduced PIZ serum level (% knockdown as compared to GFP control) (Mueller et al 2012). As described herein, positional impacts of miRNAs are tested and evaluated. In some embodiments, dsRNA-induced gene silencing can be mediated by short double-stranded small interfering RNAs (siRNAs) generated from longer dsRNAs by ribonuclease III cleavage (Bernstein et al., Nature 2001; 409:363-366 and Elbashir et al., Genes Dev. 2001; 15:188-200, the contents of each of which are hereby incorporated by reference herein in its entirety). Without being bound by any particular theory, RNAi-mediated gene silencing is thought to occur via sequence-specific RNA degradation, where sequence specificity is determined by interaction of a siRNA with its complementary sequence within a target RNA (see, e.g., Tuschl, Chem. Biochem. 2001; 2:239-245). In some embodiments, RNAi can involve use of, e.g., siRNAs (Elbashir, et al., Nature 2001; 411:494-498, which is incorporated in its entirety herein by reference) or short hairpin RNAs (shRNAs) bearing a fold back stem-loop structure (Paddison et al., Genes Dev. 2002; 16:948-958; Sui et al., Proc. Natl. Acad. Sci. USA 2002; 99:5515-5520; Brummelkamp et al., Science 2002; 296:550-553; Paul et al., Nature Biotechnol. 2002; 20:505-508, each of which is incorporated in its entirety herein by reference).

In some embodiments an inhibitory nucleic acid is one or more of a short interfering RNA (siRNA), a short hairpin RNA (shRNA), an antisense oligonucleotide, or a ribozyme. In some embodiments, knockdown of gene expression is achieved via inhibitory nucleic acids that target a target sequence as described herein. In some such embodiments, a targeted target sequence may be a wild-type and/or pathogenic variant gene product.

siRNA or shRNA

In some embodiments, the present disclosure provides an inhibitory nucleic acid e, e.g., a chemically-modified siRNAs or a construct-driven expression of short hairpin RNA (shRNA) that are then cleaved to siRNA, e.g., within a cell. Accordingly, one of skill in the art will understand that, for purposes of sequences, an shRNA sequence is interchangeable with an siRNA sequence and that where the disclosure refers to an siRNA, an shRNA sequence may be used since the shRNA will be cleaved into siRNA. For example, in some embodiments, an inhibitory nucleic acid can be a dsRNA (e.g., siRNA) including 16-30 nucleotides, e.g., 16, 17, 18, 19, 20, 21, 22, 23, 24, 25, 26, 27, 28, 29, or 30 nucleotides in each strand, where one strand is substantially identical, e.g., at least 80% (or more, e.g., 85%, 90%, 95%, or 100%) identical, e.g., having 3, 2, 1, or 0 mismatched nucleotide(s), to a target region in an mRNA, and the other strand is complementary to the first strand. In some embodiments, dsRNA molecules can be designed using methods known in the art, e.g., Dharmacon.com (see, siDESIGN CENTER) or “The siRNA User Guide,” available on the Internet at mpibpc.gwdg.de/abteilungen/100/105/sirna.html website which is incorporated in its entirety herein by reference. Without being bound by any particular theory, the present disclosure contemplates that siRNA or shRNAs are more “endogenous” (e.g., no foreign proteins) in a way that may be more recognizable to a cell compared to other available techniques that will be known to those of skill in the art. Accordingly, in some embodiments, siRNA or shRNA have lower immunogenicity and/or have less risk of off-target DNA cleavage as compared to other techniques known to those of skill in the art.

Several methods for expressing siRNA duplexes within cells from a construct to achieve long-term target gene suppression in cells are known in the art, e.g., including constructs that use a mammalian Pol III promoter system (e.g., H1 or U6/snRNA promoter systems (Tuschl, Nature Biotechnol., 20:440-448, 2002, which is incorporated in its entirety herein by reference) to express functional double-stranded siRNAs; (Bagella et al., J. Cell. Physiol., 177:206-213, 1998; Lee et al., Nature Biotechnol., 20:500-505, 2002; Paul et al., Nature Biotechnol., 20:505-508, 2002; Yu et al., Proc. Natl. Acad. Sci. U.S.A., 99(9): 6047-6052, 2002; Sui et al., Proc. Natl. Acad. Sci. U.S.A. 99(6): 5515-5520, 2002, each of which is incorporated in its entirety herein by reference). Transcriptional termination by RNA Pol III occurs at runs of four consecutive T residues in a DNA template, and can be used to provide a mechanism to end the siRNA transcript at a specific sequence. An siRNA is complementary to a sequence of a target gene in 5′-3′ and 3′-5′ orientations, and the two strands of a given siRNA can be expressed in the same construct or in separate constructs. Hairpin siRNAs, driven by H1 or U6 snRNA promoter and expressed in cells, can inhibit target gene expression (Bagella et al., 1998, supra; Lee et al., 2002, supra; Paul et al., 2002, supra; Yu et al., 2002, supra; Sui et al., 2002, supra).

In some embodiments, siRNAs of the present disclosure are double stranded nucleic acid duplexes (of, e.g., 15, 16, 17, 18, 19, 20, 21, 22, 23, 24, 25, 26, or 27 base pairs) comprising annealed complementary single stranded nucleic acid molecules. In some embodiments, siRNAs are short dsRNAs comprising annealed complementary single strand RNAs. In some embodiments, siRNAs comprise an annealed RNA: DNA duplex, wherein the sense strand of a duplex is a DNA molecule and the antisense strand of the same duplex is a RNA molecule.

In some embodiments, duplexed siRNAs comprise a 2 or 3 nucleotide 3′ overhang on each strand of a duplex. In some embodiments, siRNAs comprise 5′-phosphate and 3′-hydroxyl groups.

In some embodiments, a siRNA molecule of the present disclosure includes one or more natural nucleobase and/or one or more modified nucleobases derived from a natural nucleobase. Examples include, but are not limited to, uracil, thymine, adenine, cytosine, and guanine having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being natural degradation products). Exemplary modified nucleobases are disclosed in Chiu and Rana, R N A, 2003, 9, 1034-1048, Limbach et al. Nucleic Acids Research, 1994, 22, 2183-2196 and Revankar and Rao, Comprehensive Natural Products Chemistry, vol. 7, 313, each of which is incorporated in its entirety herein by reference.

Modified nucleobases also include expanded-size nucleobases in which one or more aryl rings, such as phenyl rings, have been added. Nucleic base replacements described in the Glen Research catalog (available on the world wide web at glenresearch.com); Krueger A T et al., Acc. Chem. Res., 2007, 40, 141-150; Kool, ET, Acc. Chem. Res., 2002, 35, 936-943; Benner S. A., et al., Nat. Rev. Genet., 2005, 6, 553-543; Romesberg, F. E., et al., Curr. Opin. Chem. Biol., 2003, 7, 723-733; Hirao, I., Curr. Opin. Chem. Biol., 2006, 10, 622-627, each of which is incorporated in its entirety herein by reference, are contemplated as useful for siRNA molecules described herein. In some embodiments, modified nucleobases also encompass structures that are not considered nucleobases but are other moieties such as, but not limited to, corrin- or porphyrin-derived rings. Porphyrin-derived base replacements have been described in Morales-Rojas, H and Kool, ET, Org. Lett., 2002, 4, 4377-4380, which is incorporated in its entirety herein by reference.

In some embodiments, modified nucleobases are of any one of the following structures, optionally substituted:

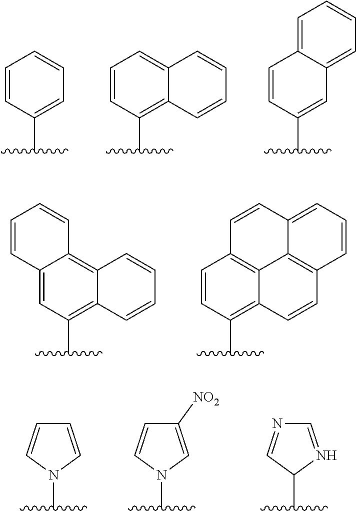

In some embodiments, a modified nucleobase is fluorescent. Exemplary such fluorescent modified nucleobases include phenanthrene, pyrene, stillbene, isoxanthine, isozanthopterin, terphenyl, terthiophene, benzoterthiophene, coumarin, lumazine, tethered stillbene, benzo-uracil, and naphtho-uracil, as shown below:

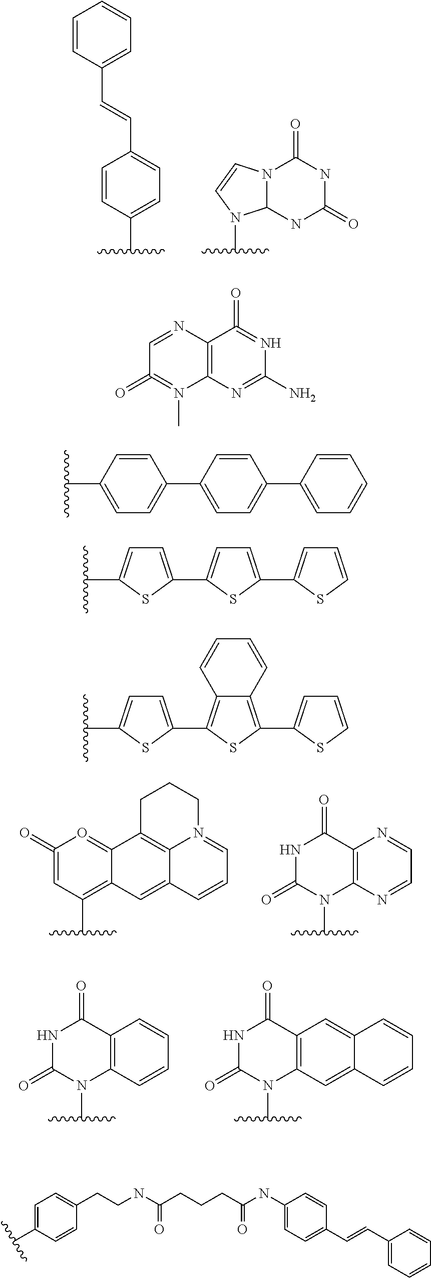

In some embodiments, a modified nucleobase is unsubstituted. In some embodiments, a modified nucleobase is substituted. In some embodiments, a modified nucleobase is substituted such that it contains, e.g., heteroatoms, alkyl groups, or linking moieties connected to fluorescent moieties, biotin or avidin moieties, or other protein or peptides. In some embodiments, a modified nucleobase is a “universal base” that is not a nucleobase in the most classical sense, but that functions similarly to a nucleobase. One representative example of such a universal base is 3-nitropyrrole.

In some embodiments, siRNA molecules described herein include nucleosides that incorporate modified nucleobases and/or nucleobases covalently bound to modified sugars. Some examples of nucleosides that incorporate modified nucleobases include 4-acetylcytidine; 5-(carboxyhydroxylmethyl) uridine; 2′-O-methylcytidine; 5-carboxymethylaminomethyl-2-thiouridine; 5-carboxymethylaminomethyluridine; dihydrouridine; 2′-O-methylpseudouridine; beta,D-galactosylqueosine; 2′-O-methylguanosine; N⁶-isopentenyladenosine; 1-methyladenosine; 1-methylpseudouridine; 1-methylguanosine; 1-methylinosine; 2,2-dimethylguanosine; 2-methyladenosine; 2-methylguanosine; N⁷-methylguanosine; 3-methyl-cytidine; 5-methylcytidine; 5-hydroxymethylcytidine; 5-formylcytosine; 5-carboxylcytosine; N⁶-methyladenosine; 7-methylguanosine; 5-methylaminoethyluridine; 5-methoxyaminomethyl-2-thiouridine; beta,D-mannosylqueosine; 5-methoxycarbonylmethyluridine; 5-methoxyuridine; 2-methylthio-N⁶-isopentenyladenosine; N-((9-beta,D-ribofuranosyl-2-methylthiopurine-6-yl) carbamoyl) threonine; N-((9-beta,D-ribofuranosylpurine-6-yl)-N-methylcarbamoyl) threonine; uridine-5-oxyacetic acid methylester; uridine-5-oxyacetic acid (v); pseudouridine; queosine; 2-thiocytidine; 5-methyl-2-thiouridine; 2-thiouridine; 4-thiouridine; 5-methyluridine; 2′-O-methyl-5-methyluridine; and 2′-O-methyluridine.

In some embodiments, nucleosides include 6′-modified bicyclic nucleoside analogs that have either (R) or(S)-chirality at the 6′-position and include the analogs described in U.S. Pat. No. 7,399,845, which is incorporated in its entirety herein by reference. In some embodiments, nucleosides include 5′-modified bicyclic nucleoside analogs that have either (R) or(S) -chirality at the 5′-position and include the analogs described in U.S. Publ. No. 20070287831, which is incorporated in its entirety herein by reference. In some embodiments, a nucleobase or modified nucleobase is 5-bromouracil, 5-iodouracil, or 2,6-diaminopurine. In some embodiments, a nucleobase or modified nucleobase is modified by substitution with a fluorescent moiety.

Methods of preparing modified nucleobases are described in, e.g., U.S. Pat. Nos. 3,687,808; 4,845,205; 5,130,30; 5,134,066; 5,175,273; 5,367,066; 5,432,272; 5,457,187; 5,457,191; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is incorporated in its entirety herein by reference.

In some embodiments, a siRNA molecule described herein includes one or more modified nucleotides wherein a phosphate group or linkage phosphorus in its nucleotides are linked to various positions of a sugar or modified sugar. As non-limiting examples, a phosphate group or linkage phosphorus can be linked to a 2′, 3′, 4′ or 5′ hydroxyl moiety of a sugar or modified sugar. Nucleotides that incorporate modified nucleobases as described herein are also contemplated in this context.

Other modified sugars can also be incorporated within a siRNA molecule. In some embodiments, a modified sugar contains one or more substituents at a 2′ position including one of the following: —F; CF₃, —CN, —N³, —NO, —NO₂, —OR′, —SR′, or —N(R′)₂, wherein each R′ is independently as defined above and described herein; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH (C₁-C₁₀ alkyl)₂, —NH-(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein the alkyl, alkylene, alkenyl and alkynyl may be substituted or unsubstituted. Examples of substituents include, and are not limited to, —O(CH₂)ₙOCH₃, and —O(CH₂)ₙNH₂, wherein n is from 1 to about 10, MOE, DMAOE, DMAEOE. Also contemplated herein are modified sugars described in WO 2001/088198; and Martin et al., Helv. Chim. Acta, 1995, 78, 486-504, each of which is incorporated in its entirety herein by reference. In some embodiments, a modified sugar comprises one or more groups selected from a substituted silyl group, an RNA cleaving group, a reporter group, a fluorescent label, an intercalator, a group for improving pharmacokinetic properties of a nucleic acid, a group for improving pharmacodynamic properties of a nucleic acid, or other substituents having similar properties. In some embodiments, modifications are made at one or more of a 2′, 3′, 4′, 5′, or 6′ positions of a sugar or modified sugar, including a 3′ position of a sugar on a 3′-terminal nucleotide or in a 5′ position of a 5′-terminal nucleotide.

In some embodiments, a 2′-OH of a ribose is replaced with a substituent including one of the following: —H, —F; —CF₃, —CN, —N³, —NO, —NO₂, —OR′, —SR′, or —N(R′)₂, wherein each R′ is independently as defined above and described herein; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH (C₁-C₁₀ alkyl)₂, —NH-(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein an alkyl, alkylene, alkenyl and alkynyl may be substituted or unsubstituted. In some embodiments, a 2′-OH is replaced with —H (deoxyribose). In some embodiments, a 2′-OH is replaced with —F. In some embodiments, a 2′-OH is replaced with —OR′. In some embodiments, a 2′-OH is replaced with —OMe. In some embodiments, a 2′-OH is replaced with —OCH₂CH₂OMe.

Modified sugars also include locked nucleic acids (LNAs). In some embodiments, a locked nucleic acid has the structure indicated below. A locked nucleic acid of the structure below is indicated, wherein Ba represents a nucleobase or modified nucleobase as described herein, and wherein R^{2s} is —OCH₂C₄′—

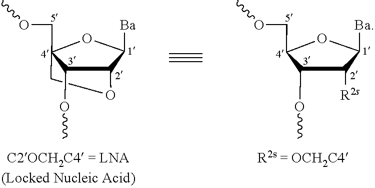

In some embodiments, a modified sugar is an ENA such as those described in, e.g., Seth et al., J Am Chem Soc. 2010 Oct. 27; 132(42): 14942-14950, which is incorporated in its entirety herein by reference. In some embodiments, a modified sugar is any of those found in an XNA (xenonucleic acid), for instance, arabinose, anhydrohexitol, threose, 2′fluoroarabinose, or cyclohexene.

Modified sugars include sugar mimetics such as cyclobutyl or cyclopentyl moieties in place of the pentofuranosyl sugar (see, e.g., U.S. Pat. Nos. 4,981,957; 5,118,800; 5,319,080; and 5,359,044, each of which is incorporated in its entirety herein by reference). Some modified sugars that are contemplated include sugars in which an oxygen atom within a ribose ring is replaced by nitrogen, sulfur, selenium, or carbon. In some embodiments, a modified sugar is a modified ribose wherein an oxygen atom within a ribose ring is replaced with nitrogen, and wherein a nitrogen is optionally substituted with an alkyl group (e.g., methyl, ethyl, isopropyl, etc.).

Non-limiting examples of modified sugars include glycerol, which form glycerol nucleic acid (GNA) analogues. An exemplary GNA analogue is described in Zhang, R et al., J. Am. Chem. Soc., 2008, 130, 5846-5847, which is incorporated in its entirety herein by reference; see also Zhang L, et al., J. Am. Chem. Soc., 2005, 127, 4174-4175 and Tsai C H et al., PNAS, 2007, 14598-14603, each which is incorporated in its entirety herein by reference. Another example of a GNA derived analogue, flexible nucleic acid (FNA) based on mixed acetal aminal of formyl glycerol, is described in each of Joyce G F et al., PNAS, 1987, 84, 4398-4402 and Heuberger BD and Switzer C, J. Am. Chem. Soc., 2008, 130, 412-413, each of which is incorporated in its entirety herein by reference. Additional non-limiting examples of modified sugars include hexopyranosyl (6′ to 4′), pentopyranosyl (4′ to 2′), pentopyranosyl (4′ to 3′), or tetrofuranosyl (3′ to 2′) sugars.

Modified sugars and sugar mimetics can be prepared by methods known in the art, including, but not limited to: A. Eschenmoser, Science (1999), 284:2118; M. Bohringer et al., Helv. Chim. Acta (1992), 75:1416-1477; M. Egli et al., J. Am. Chem. Soc. (2006), 128(33): 10847-56; A. Eschenmoser in Chemical Synthesis: Gnosis to Prognosis, C. Chatgilialoglu and V. Sniekus, Ed., (Kluwer Academic, Netherlands, 1996), p.293; K.-U. Schoning et al., Science (2000), 290:1347-1351; A. Eschenmoser et al., Helv. Chim. Acta (1992), 75:218; J. Hunziker et al., Helv. Chim. Acta (1993), 76:259; G. Otting et al., Helv. Chim. Acta (1993), 76:2701; K. Groebke et al., Helv. Chim. Acta (1998), 81:375; and A. Eschenmoser, Science (1999), 284:2118. Modifications to 2′ modifications can be found in Verma, S. et al. Annu. Rev. Biochem. 1998, 67, 99-134 and all references therein, each of which is incorporated in its entirety herein by reference. Specific modifications to a ribose can be found in the following references: 2′-fluoro (Kawasaki et. al., J. Med. Chem., 1993, 36, 831-841), 2′-MOE (Martin, P. Helv. Chim. Acta 1996, 79, 1930-1938), “LNA” (Wengel, J. Acc. Chem. Res. 1999, 32, 301-310); PCT Publication No. WO2012/030683, each of which is incorporated in its entirety herein by reference.

In some embodiments, a siRNA described herein can be introduced to a target cell as an annealed duplex siRNA. In some embodiments, a siRNA described herein is introduced to a target cell as single stranded sense and antisense nucleic acid sequences that, once within a target cell, anneal to form a siRNA duplex. Alternatively, sense and antisense strands of an siRNA can be encoded by an expression construct (such as an expression construct described herein) that is introduced to a target cell. Upon expression within a target cell, transcribed sense and antisense strands can anneal to reconstitute an siRNA.

In some embodiments, an siRNA molecule as described herein can be synthesized by standard methods known in the art, e.g., by use of an automated synthesizer. Without being bound by any particular theory, RNAs produced by such methodologies tend to be highly pure and to anneal efficiently to form siRNA duplexes. In some embodiments, following chemical synthesis, single stranded RNA molecules can be deprotected, annealed to form siRNAs, and purified (e.g., by gel electrophoresis or HPLC). Alternatively, in some embodiments, standard procedures can be used for in vitro transcription of RNA from DNA templates, e.g., carrying one or more RNA polymerase promoter sequences (e.g., T7 or SP6 RNA polymerase promoter sequences). Protocols for preparation of siRNAs using T7 RNA polymerase are known in the art (see, e.g., Donze and Picard, Nucleic Acids Res. 2002; 30: e46; and Yu et al., Proc. Natl. Acad. Sci. USA 2002; 99:6047-6052, each of which is incorporated in its entirety herein by reference). In some embodiments, sense and antisense transcripts can be synthesized in two independent reactions and annealed later. In some embodiments, sense and antisense transcripts can be synthesized simultaneously in a single reaction.

In some embodiments, an siRNA molecule can also be formed within a cell by transcription of RNA from an expression construct introduced into a cell (see, e.g., Yu et al., Proc. Natl. Acad. Sci. USA 2002; 99:6047-6052, which is incorporated in its entirety herein by reference). For example, in some embodiments, an expression construct for in vivo production of siRNA molecules can include one or more siRNA encoding sequences operably linked to elements necessary for proper transcription of an siRNA encoding sequence(s), including, e.g., promoter elements and transcription termination signals. In some embodiments, preferred promoters for use in such expression constructs may include, e.g., a polymerase-III promoter, e.g., a polymerase-III HI-RNA promoter (see, e.g., Brummelkamp et al., Science 2002; 296:550-553, which is incorporated in its entirety herein by reference), a U6 polymerase-III promoter (see, e.g., Sui et al., Proc. Natl. Acad. Sci. USA 2002; Paul et al., Nature Biotechnol. 2002; 20:505-508; and Yu et al., Proc. Natl. Acad. Sci. USA 2002; 99:6047-6052, each of which is incorporated in its entirety herein by reference). In some embodiments, an siRNA expression construct can comprise one or more construct sequences that facilitate cloning of an expression construct. Standard constructs that can be used include, e.g., pSilencer 2.0-U6 construct (Ambion Inc., Austin, Tex.).

miRNA

The present disclosure provides technologies related to or comprising one or more inhibitory nucleic acid molecules such as, e.g., one or more nucleotide sequences that are, comprise, or encode, microRNAs. MicroRNAs (miRNAs) are a highly conserved class of small RNA molecules that are transcribed from DNA in genomes of plants and animals, but are not translated into protein. As is known to those in the art, animal cells express a range of noncoding RNAs of approximately 22 nucleotides termed micro RNA (miRNAs) and can regulate gene expression at a post transcriptional or translational level during animal development. miRNAs are excised from an approximately 70 nucleotide precursor RNA stem-loop. By substituting stem sequences of an miRNA precursor with miRNA sequence complementary to a target mRNA, a construct that expresses a novel miRNA can be used to produce siRNAs to initiate RNAi against specific mRNA targets in mammalian cells (Zeng, Mol. Cell, 9:1327-1333, 2002). In some embodiments, when expressed by DNA constructs containing polymerase III promoters, micro-RNA designed hairpins can silence gene expression (McManus, RNA 8:842-850, 2002).

In some embodiments, miRNAs can be synthesized and locally or systemically administered to a subject, e.g., for therapeutic purposes. In some embodiments, miRNAs can be designed and/or synthesized as mature molecules or precursors (e.g., pri- or pre-miRNAs). In some embodiments, a pre-miRNA includes a guide strand and a passenger strand that are the same length (e.g., about 15, 16, 17, 18, 19, 20, 21, 22, 23, 24, or 25 nucleotides). In some embodiments, a pre-miRNA includes a guide strand and a passenger strand that are different lengths (e.g., one strand is about 19 nucleotides, and the other is about 21 nucleotides). In some embodiments, an miRNA can target a coding region, a 5′ untranslated region, and/or a 3′ untranslated region, of endogenous mRNA. In some embodiments, an miRNA comprises a guide strand comprising a nucleotide sequence having sufficient sequence complementary with an endogenous mRNA of a subject to hybridize with and inhibit expression of endogenous mRNA.

### Antisense Nucleic Acid

In some embodiments, an inhibitory nucleic acid molecule may be or comprise an antisense nucleic acid molecule, e.g., nucleic acid molecules whose nucleotide sequence is complementary to all or part of an mRNA encoding a protein of interest. In some embodiments, a non-coding regions (“5′ and 3′ untranslated regions”) are 5′ and 3′ sequences that flank a coding region and are not translated into amino acids. Based upon sequences disclosed herein, one of skill in the art can easily choose and synthesize any of a number of appropriate antisense molecules to target a gene as described herein. For example, a “gene walk” comprising a series of oligonucleotides of 15-30 nucleotides spanning a length of a nucleic acid (e.g., an mRNA) can be prepared, followed by testing for inhibition of expression of a gene. Optionally, gaps of 5-10 nucleotides can be left between oligonucleotides to reduce numbers of oligonucleotides synthesized and tested.

In some embodiments, an antisense oligonucleotide can be, for example, about 5, 10, 15, 20, 25, 30, 35, 40, 45, or 50 nucleotides or more in length. One of skill in the art will recognize that an antisense oligonucleotide can be synthesized using various different chemistries.

### Ribozymes

In some embodiments, an inhibitory nucleic acid molecule may be or comprise a ribozyme. As is known to those of skill in the art, ribozymes are catalytic RNA molecules with ribonuclease activity. In some embodiments, a ribozyme may be used as a controllable promoter. In some embodiments, ribozymes are capable of cleaving a single-stranded nucleic acid, such as an mRNA, to which they have a complementary region. Thus, in some embodiments, ribozymes (e.g., hammerhead ribozymes (described in Haselhoff and Gerlach, Nature, 334:585-591, 1988, which is incorporated in its entirety herein by reference)) can be used to catalytically cleave mRNA transcripts to thereby inhibit translation of a protein encoded by a given mRNA. Methods of designing and producing ribozymes are known in the art (see, e.g., Scanlon, 1999, Therapeutic Applications of Ribozymes, Humana Press, which is incorporated in its entirety herein by reference). In some embodiments, for example, a ribozyme having specificity for a transgene mRNA can be designed based upon nucleotide sequence of a transgene gene product cDNA (e.g., any exemplary cDNA sequences described herein). For example, a derivative of a Tetrahymena L-19 IVS RNA can be constructed in which nucleotide sequence of an active site is complementary to a nucleotide sequence to be cleaved in a transgene mRNA (Cech et al. U.S. Pat. No. 4,987,071; and Cech et al., U.S. Pat. No. 5,116,742, each of which is incorporated in its entirety herein by reference). Alternatively, an mRNA encoding a transgene protein can be used to select a catalytic RNA having a specific ribonuclease activity from a pool of RNA molecules (See, e.g., Bartel and Szostak, Science, 261:1411-1418, 1993, which is incorporated in its entirety herein by reference).

i. Pharmaceutical Compositions and Kits

Pharmaceutical compositions of the present disclosure may include constructs, as described herein. For example, in some embodiments, pharmaceutical compositions may comprise constructs and/or virions. In some such embodiments, such virions comprise one or more constructs, which comprise a nucleic acid, e.g., one or a plurality of constructs described herein. For example, a pharmaceutical composition of the present disclosure comprise as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose, or dextrans; mannitol; proteins; polypeptides or amino acids such as glycine; antioxidants; chelating agents such as EDTA or glutathione; adjuvants (e.g., aluminum hydroxide); and preservatives. In some embodiments, compositions of the present disclosure are formulated for intravenous administration.

In some embodiments, a composition includes a pharmaceutically acceptable carrier (e.g., phosphate buffered saline, saline, or bacteriostatic water). Upon formulation, solutions can be administered in a manner compatible with a dosage formulation and in such amount as is therapeutically effective. Formulations are easily administered in a variety of dosage forms such as injectable solutions, injectable gels, drug-release capsules, and the like.

Compositions provided herein can be, e.g., formulated to be compatible with their intended route of administration. A non-limiting example of an intended route of administration is local administration.

Also provided are kits including any compositions or constructs described herein. In some embodiments, a kit can include a solid composition (e.g., a lyophilized composition including at least one construct as described herein) and a liquid for solubilizing a lyophilized composition. In some embodiments, a kit can include one or more constructs described herein.

In some embodiments, a kit can include a pre-loaded syringe including any compositions described herein.

In some embodiments, a kit includes a vial comprising any of the compositions described herein (e.g., formulated as an aqueous composition, e.g., an aqueous pharmaceutical composition).

In some embodiments, a kit can include instructions for performing any methods described herein.

i. Cells

In some embodiments, the present disclosure provides a cell (e.g., an insect cell, e.g., a Sf9 cell, e.g., a mammalian cell, e.g., a human cell, e.g., a HEK293T cells, etc.) that comprises any nucleic acids, constructs (e.g., at least two different constructs described herein), compositions, etc., as described herein. As will be appreciated by one of skill in the art, nucleic acids and constructs described herein can be introduced into any cell (e.g., an insect cell, e.g., a Sf9 cell, etc.). Non-limiting examples of certain constructs and methods for introducing constructs into cells are described herein.

In some embodiments, the present disclosure provides a cell (e.g., a mammalian cell, e.g., a human cell, etc.) that comprises any nucleic acids, constructs (e.g., at least two different constructs described herein), compositions, etc., as described herein. As will be appreciated by one of skill in the art, nucleic acids and constructs described herein can be introduced into any cell (e.g., a mammalian cell, e.g., a human cell, etc.). Non-limiting examples of certain constructs and methods for introducing constructs into cells are described herein.

In some embodiments, a cell is a human cell, a mouse cell, a porcine cell, a rabbit cell, a dog cell, a rat cell, a sheep cell, a cat cell, a horse cell, a non-human primate cell, or an insect cell.

In some embodiments, a cell is a primary cell (e.g., a human primary cell). In some embodiments, a cell is a liver cell. In some embodiments, a cell is a primary hepatocyte cell (e.g., a Huh7 cell). In some embodiments, a cell is a neuron cell. In some embodiments, a cell is a kidney cell (e.g., a human renal proximal tubule (HRCE) cell, e.g., a bile duct cell, e.g., an outer medullary cell, e.g., a mixed medullary cell, e.g., renal cortical epithelial cells, e.g., renal epithelial cells). In some embodiments, a cell is an immune cell. In some embodiments, a cell is a human T cell (e.g., a CD4+ T cell, e.g., a Th2 cell). In some embodiments, a cell is a blood cell (e.g., a PBMC cell). In some embodiments, a cell is a skeletal muscle cell. In some embodiments, a cell is a differentiated skeletal muscle cell (e.g., a myotube cell). In some embodiments, a cell is a primary cardiomyocyte cell. In some embodiments, a cell is a bone marrow MSC cell. In some embodiments, a cell is a small intestine cell. In some embodiments, a cell is a muscle cell. In some embodiments, a cell is a heart cell. In some embodiments, a cell is a spleen cell. In some embodiments, a cell is a brain cell (e.g., a brain-striatum cell, e.g., a neuroblastoma cell (e.g., a SH-SY5Y cell), e.g., a CD105-positive endothelial cell, e.g., a brain cortex cell).

In some embodiments, a cell is a PymT tumor cell, a cervix cancer cell (e.g., a HeLa cell), a K562 cell, a Raji cell, a SKOV-3 cell, a breast cancer cell (e.g., a MCF-7 cell), a M07e cell, a human saphenous vascular endothelial cell (HSaVEC), a MT1-MMP cell, a primary hepatocyte cell (e.g., a Huh7 cell), an immune cell (e.g., a human T cell, e.g., a CD4+ T cell, e.g., a Th2 cell, e.g., a CAR T cell, e.g., a NK cell), a neuron cell (e.g., a LX-2 cell, e.g., a stellate cell, e.g., a primary neuron cell, e.g., a neuroblastoma cell (e.g., a SH-SY5Y cell)), a lung cell (e.g., a lung fibroblast cell), a myoblast cell, a myotube cell, a primary cardiomyocyte, a skeletal muscle cell (e.g., a differentiated skeletal muscle cell), a human vein endothelial cell, a T84 cell, a ileum cell (intestinal), a primary human airway epithelia cell), a kidney cell (e.g., a human renal proximal tubule (HRCE) cell, e.g., a bile duct cell, e.g., an outer medullary cell, e.g., a mixed medullary cell, e.g., renal cortical epithelial cells, e.g., renal epithelial cells), a bone marrow MSC cell, a blood cell (e.g., a PBMC cell), a small intestine cell, a muscle cell, a heart cell, a spleen cell, a liver cell, a brain cell (e.g., a brain-striatum cell, e.g., a CD105-positive endothelial cell, e.g., a brain cortex cell) or an ocular cell. In some embodiments, a cell is a testes cell. In some embodiments, a cell is an oocyte. In some embodiments, a cell is a medulla cell. In some embodiments, a cell is a striatum cell. In some embodiments, a cell is a spinal cord (or chord) cell. In some embodiments, a cell is a duodenum cell.

In some embodiments, a cell is in vitro. In some embodiments, a cell is in vivo or ex vivo. For example, in some embodiments, cell is present in a mammal. In some embodiments, a cell (e.g., a mammalian cell) is autologous cell obtained, e.g., from a subject (e.g., a mammal) and cultured ex vivo.

In some embodiments, cells provided by the present disclosure are transfected host cells. In some embodiments, transfection is used to refer to uptake of foreign DNA by a cell, and a cell has been “transfected” when exogenous DNA has been introduced inside a cell membrane. A number of transfection techniques are generally known in the art (see, e.g., Graham et al. (1973) Virology, 52:456; Sambrook et al. (1989) Molecular Cloning, a laboratory manual, Cold Spring Harbor Laboratories, New York, Davis et al. (1986) Basic Methods in Molecular Biology, Elsevier; and Chu et al. (1981) Gene 13:197, each of which is incorporated in its entirety herein by reference). Such techniques can be used to introduce one or more exogenous nucleic acids, such as a nucleotide integration construct and other nucleic acid molecules, into suitable host cells.

### 3. Methods

Among other things, the present disclosure provides methods. In some embodiments, a method comprises producing a virion described herein. In some embodiments, a method comprises purifying a virion described herein. In some embodiments, a method comprises characterizing a virion described herein. In some embodiments, a method comprises manufacturing a virion described herein.

In some embodiments, a method comprises introducing a composition as described herein into a cell of a subject. For example, provided herein are methods that in some embodiments include administering to a cell of a subject (e.g., an animal, e.g., a mammal, e.g., a primate, e.g., a human) a therapeutically effective amount of any composition described herein.

a. Methods of Making

Among other things, the present disclosure provides for methods of making constructs described herein. For example, in some embodiments, constructs are prepared using a standard dual transfection system (e.g., two plasmids/constructs, comprising (i) rep/cap genes, (ii) helper genes, and (iii) payloads (e.g., a transgene) respectively) followed by standard isolation and purification methods (e.g., CsCl gradient). For example, in some embodiments, constructs are prepared using a standard triple transfection system (e.g., three plasmids/constructs, comprising (i) rep/cap genes, (ii) helper genes, and (iii) payloads (e.g., a transgene) respectively, e.g., four plasmids/constructs, etc.) followed by standard isolation and purification methods (e.g., CsCl gradient). In some such embodiments, such preparations are formulated for delivery into a subject.

Moreover, the present disclosure provides, among other things, a method of making protoparvovirus-related compositions, preparations, constructs, virions, populations of virions, etc. In some embodiments, such methods include use of host cells.

In some embodiments, a host cell is a mammalian cell. In some embodiments, a mammalian cell is a human cell. In some embodiments, a mammalian cell is a HEK293T cell. In some embodiments, a mammalian cell is a K562 cell. In some embodiments, a mammalian cell is a HRCE cell. For example, in some embodiments, such methods include use of an exemplary CPV construct described herein (e.g., SEQ ID NO: 130, e.g., SEQ ID NO: 142) for production of compositions, preparations, constructs, virions, populations of virions, etc. in mammalian cells (e.g. HEK293T cells). For example, in some embodiments, such methods include use of an exemplary CuV construct described herein (e.g. SEQ ID NO: 133, e.g., SEQ ID NO: 143, e.g., SEQ ID NO: 148) for production of compositions, preparations, constructs, virions, populations of virions, etc. in mammalian cells (e.g., HEK293T cells). For example, in some embodiments, such methods include use of an exemplary CuV construct described herein (e.g., SEQ ID NO: 135) for production of compositions, preparations, constructs, virions, populations of virions, etc. in mammalian cells (e.g. in HEK cells). In some embodiments, a host cell is an insect cell. In some embodiments, an insect cell is an Sf9 cell. The term includes progeny of an original cell that has been transfected. Thus, a “host cell” as used herein may refer to a cell that has been transfected with an exogenous DNA sequence. It is understood that progeny of a single parental cell may not necessarily be completely identical in morphology or in genomic or total DNA complement as the original parent, due to natural, accidental, or deliberate mutation.

In some embodiments, the present disclosure provides a system in which a transgene flanked by ITRs and rep/cap genes are introduced into insect host cells by infection with insect virus (e.g., baculovirus)-based constructs. Such production systems are known in the art.

In some embodiments, provided herein are methods of producing a virion or a population of virions described herein. A number of constructs described herein may be consolidated by incorporating the structural and/or nonstructural genes into one or more constructs. In some embodiments, certain protoparvovirus genomic sequence(s) may also be integrated into a baculovirus genome to contain structural (e.g., encoding VP polypeptides(s)) and/or nonstructural genes. In some embodiments, certain protoparvovirus genomic sequences may also be integrated into a mammalian genome to contain structural (e.g., encoding VP polypeptides(s)) and/or nonstructural genes.

In some embodiments, provided herein are methods of producing a virion having a protoparvovirus variant VP1 capsid polypeptide, wherein the protoparvovirus is of a species selected from Carnivore protoparvovirus, Carnivore protoparvovirus 1, Chiropteran protoparvovirus 1, Eulipotyphla protoparvovirus 1, Primate protoparvovirus 1, Primate protoparvovirus 2, Primate protoparvovirus 3, Primate protoparvovirus 4, Rodent protoparvovirus 1, Rodent protoparvovirus 2, Rodent protoparvovirus 3, Ungulate protoparvovirus 1, and Ungulate protoparvovirus 2. In some embodiments, protoparvovirus is selected from canine parvovirus, feline panleukopenia virus, human bufavirus 1, human bufavirus 2, human bufavirus 3, human tusavirus, human cutavirus, Wuharv parvovirus, porcine parvovirus, minute virus of mice, or megabat bufavirus, or genetic variant thereof.

In some embodiments, an insect cell is derived from a species of lepidoptera, e.g., Spodoptera frugiperda, Spodoptera littoralis, Spodoptera exigua, or Trichoplusiani. In some embodiments, an insect cell is Sf9. In some embodiments, a construct is a baculoviral construct, a viral construct, or a plasmid. In some embodiments, the at least one construct is a baculoviral construct. In some embodiments, subclones of lepidopteran cell lines that demonstrate enhanced virion yield on a per cell or per volume basis are used. In some embodiments, modified lepidopteran cell lines with an integrated copy of NS1, Rep, VP, and/or construct genome, singly or in combinations, are used. The insect cell line, in some embodiments, is “cured” of endogenous or contaminating or adventitious insect viruses such as the Spodoptera rhabdovirus.

In some embodiments, a virion may also be produced using a mammalian cell, e.g., Grieger et al (2016) Mol Ther 24:287-297, the contents of which are incorporated by reference herein in its entirety).

b. Methods of Treatment

Among other things, in some embodiments, technologies of the present disclosure are used to treat a disease or disorder. In some embodiments, provided herein are methods of preventing or treating a disease using a virion or pharmaceutical compositions described herein. In some embodiments, a virion disclosed herein provides to a subject a transgene (e.g., those encoding a therapeutic protein or a fragment thereof) transiently, e.g., a nucleic acid transduced by a virion is eventually lost after a certain period of expression. In preferred embodiments, a nucleic acid transduced by a virion integrates stably inside cells.

In some embodiments, provided herein are methods of preventing or treating a disease, comprising administering to a subject in need thereof an effective amount of a virion or pharmaceutical composition of the present disclosure. In some embodiments, a nucleic acid encodes a polypeptide. In some embodiments, a nucleic acid decreases or eliminates expression of an endogenous gene. In some embodiments, provided herein are methods of preventing or treating a disease, comprising: (a) administering to a subject in need thereof an effective amount of a virion described herein comprising a nucleic acid that increases or restores expression of a gene whose endogenous expression is aberrantly lower than expression in a healthy subject; or (b) administering to a subject in need thereof an effective amount of a virion described herein comprising a nucleic acid that decreases or eliminates expression of a gene whose endogenous expression is aberrantly higher than expression in a healthy subject. In some embodiments, a nucleic acid comprises a transgene.

In some embodiments, provided herein are methods of preventing or treating a disease, comprising: (a) obtaining a plurality of cells from a subject with disease, (b) transducing cells with a virion described herein, optionally further selecting or screening for transduced cells, and (c) administering an effective amount of transduced cells to a subject. In some embodiments, cells are autologous to a subject. In some embodiments, cells are allogeneic to a subject. There are advantages of preparing transduced cells in vitro or ex vivo. First, existence and location of a transgene in a target cell genome can be verified before administering them to a patient, thereby avoiding interfering with cell functions or off target effects. This improves safety, even without the use of GSH. Second, transduced cells can be administered to a subject in need thereof without a virion. This can eliminate any concern for triggering immune response or inducing neutralizing antibodies that inactivate virion. Accordingly, transduced cells can be safely redosed or the dose can be titrated without any adverse effect.

Among other things, in some embodiments, provided herein are methods of preventing or treating a disease comprising standard of care measures used for gene therapies described in the art. In some embodiments, a virion or population of virions, a pharmaceutical composition, or transduced cells described herein can induce an immune response in a subject. In some embodiments, provided herein are methods of preventing or treating a disease, comprising, among other things, co-administering to a subject (1) an immune suppressant and/or a prophylactic and (2) a virion or population of virions, a pharmaceutical composition, or transduced cells described herein to mitigate an immune response. In some embodiments, a disease is an exemplary disease described herein. In some embodiments, a disease is not an ocular disease. In some embodiments, an immune suppressant and/or a prophylactic is administered to a subject prior to administering to a subject a virion or population of virions, a pharmaceutical composition, or transduced cells. In some embodiments, an immune suppressant and/or a prophylactic is administered to a subject after administering to a subject a virion or population of virions, a pharmaceutical composition, or transduced cells. In some embodiments, an immune suppressant and/or a prophylactic is administered to a subject at the same time as administering to a subject a virion or population of virions, a pharmaceutical composition, or transduced cells.

In some embodiments of any methods described herein, such methods may result in improvement in a disease described herein (e.g., any metrics for determining improvement in a disease described herein) in a subject in need thereof for at least 10 days, at least 15 days, at least 20 days, at least 25 days, at least 30 days, at least 35 days, at least 40 days, at least 45 days, at least 50 days, at least 55 days, at least 60 days, at least 65 days, at least 70 days, at least 75 days, at least 80 days, at least 85 days, at least 100 days, at least 105 days, at least 110 days, at least 115 days, at least 120 days, at least 5 months, at least 6 months, at least 7 months, at least 8 months, at least 9 months, at least 10 months, at least 11 months, or at least 12 months.

In some embodiments, a virion, pharmaceutical composition, or transduced cells of the present disclosure are administered via intravascular, intracerebral, parenteral, intraperitoneal, intravenous, epidural, intraspinal, intrasternal, intra-articular, intra-synovial, intrathecal, intra-arterial, intracardiac, intramuscular, intranasal, intrapulmonary, skin graft, or oral administration.

In some embodiments, provided herein are methods of preventing or treating a hemoglobinopathy, comprising: (a) administering to a subject in need thereof an effective amount of a virion described herein, comprising a nucleic acid that encodes a hemoglobin subunit, or (b) obtaining erythroid-lineage cells or bone marrow cells from a subject in need thereof, transducing the cells with a virion described herein, comprising a nucleic acid that encodes a hemoglobin subunit, optionally further selecting or screening for transduced cells; and administering an effective amount of cells to a subject. In some embodiments, the hemoglobinopathy is beta-thalassemia or sickle cell disease.

In some embodiments, provided herein are methods of preventing or treating a disease using a virion or pharmaceutical composition comprising a protoparvovirus variant VP1 capsid polypeptide.

As described herein, protoparvovirus transduces cells via its interaction with transferrin receptors (TfR) that are expressed on the target cells. It is an insight of the present disclosure that a mouse transferrin receptor is similar to a human transferrin receptor. In some embodiments, a target cell is a mouse cell comprising a human transferrin receptor. In some embodiments, preparations, constructs, virions, or population of virions described herein are administered to a mouse comprising a human transferrin receptor.

TfR or CD71 is expressed in brain microvascular endothelial cells (BMVECs) the major element of the blood-brain barrier (BBB) (Navone, Marfia et al. 2013 the entire contents of which are hereby incorporated by reference herein). The blood-brain barrier (BBB) constitutes a primary limitation for passage of substances, both soluble and cellular, from the blood into the brain. CD71 has become an alternative to drive receptor specific transcystosis and deliver macromolecules such as antibodies to a brain parenchyma. Thus, protoparvovirus (e.g., CPV) can exploit the use of CD71 to translocate to a brain via systemic administration and transduce brain cells to prevent or treat different neurodegenerative disorders and neuromuscular disorders including but not limited to spinal muscular atrophy type 1, Huntington's disease, Canavan's disease, and lysosomal storage diseases. TfR or CD71 is also highly expressed in erythroid progenitor cells at early stage during differentiation and B lymphoblast cells. CD71 expression transiently overlap with CD34 expression in progenitor cells, before differentiation to lymphoid or erythroid lineages. Thus, protoparvovirus (e.g., CPV) can transduce stem cells and be used for T cells, B cells or NK cells derived therapies after differentiation from stem cells. Some of these uses are in cancer therapy, antimicrobial or autoimmunity related therapies. After HSC differentiation to myeloid progenitors lineage, CD71/TfR is highly expressed in basophilic Endemic Burkitt lymphoma (EBL), polychromatic erythroblast and orthochromatic erythroblasts during erythropoiesis, before the final step to produce non-nucleated erythrocytes, therefore protoparvovirus (e.g., CPV) compositions can be used for treatment or prevention of non-malignant hemoglobinopathies such as sickle cell disease by expressing anti-sickling versions of hemoglobin genes. In some embodiments, provided herein are methods of preventing or treating a disease using a virion comprising a variant VP1 capsid polypeptide or a variant thereof of a bufavirus, cutavirus, or tusavirus. Bufavirus, cutavirus, tusavirus, or a virion comprising a variant capsid polypeptide of any one of said viruses, has broad applications for gastrointestinal disorders and other target tissues. For instance, cutavirus has been isolated from skin samples in patients with cutaneous T cells lymphomas and melanomas, showing a tropism for T and B cells. Such tropism makes cutavirus attractive for gene transfer applications in lymphoid progenitor cells and subsequent applications (i) in differentiated T cells such as CAR-T and related cancer therapies, or (ii) in differentiated B cells and their applications to express therapeutic human antibodies against invading pathogens, tumor cells (e.g., tumor antigens or neoantigens), or chronic autoimmune disease.

In some embodiments, a virion comprises a variant VP1 capsid polypeptide(s) of a cutavirus. In some embodiments, a virion or pharmaceutical composition targets a T cell, B cell, and/or a lymphoid progenitor cell. In some embodiments, a virion, pharmaceutical composition, or transduced cells prevent or treat cancer.

In some embodiments, a virion, a population of virions, a composition, or a pharmaceutical composition comprises a transgene coding sequence encoding a protein or a fragment thereof selected from a hemoglobin gene (HBA1, HBA2, HBB, HBG1, HBG2, HBD, HBE1, and/or HBZ), a gene encoding an alpha-hemoglobin stabilizing protein (AHSP), coagulation factor VIII, coagulation factor IX, von Willebrand factor, dystrophin or truncated dystrophin, micro-dystrophin, utrophin or truncated utrophin, micro-utrophin, usherin (USH2A), CEP290, glial cell line-derived neurotrophic factor (GDNF), neuturin (NTN), HTT, neuronal apoptosis inhibitory protein (NAIP), INS, F8 or a fragment thereof (e.g., fragment encoding B-domain deleted polypeptide (e.g., VIII SQ, p-VIII)), cystic fibrosis transmembrane conductance regulator (CFTR), a gene associated with Alport syndrome (e.g., Col4a3, Col4a4, Col4a5), a gene associated with Fabry disease (e.g., GLA), a gene associated with autosomal dominant polycystic kidney disease (PKD) (e.g., PKD, PKD1, PKD2), a gene associated with congenital nephrotic syndrome (e.g., NPHS1 (Nephrin), NPHS2 (Podocin), a gene associated with hypertrophic cardiomyopathy (e.g., MYBPC3, JPH2, ALPK3), a gene associated with dilated cardiomyopathy (e.g., RBM20), or a gene associated with dilated cardiomyopathy (e.g., ALPK3, LMNA, BAG3).

In some embodiments, a virion, population of virions, preparation, composition, or pharmaceutical composition transduces (a) a CD34+ stem cell, optionally transduces ex vivo; (b) a mesenchymal stem cell, optionally transduces ex vivo; (c) a liver cell, (d) a small intestinal cell, and/or (e) a lung cell.

In some embodiments, a virion, population of virions, preparation, composition, or pharmaceutical composition transduces a mammalian cell. In some embodiments, a virion, population of virions, preparation, composition, or pharmaceutical composition transduces a human cell. In some embodiments, a virion, population of virions, preparation, composition, or pharmaceutical composition transduces a human kidney cell. In some embodiments, a virion, population of virions, preparation, composition, or pharmaceutical composition transduces a myeloid cell. In some embodiments, a virion, a population of virions, a preparation, a composition, or a pharmaceutical composition transduces a cardiac cell. In some embodiments, a virion, a population of virions, a preparation, a composition, or a pharmaceutical composition transduces a brain cell.

In some embodiments, a virion, population of virions, preparation, composition, or pharmaceutical composition comprises a nucleic encoding (a) CFTR or a fragment thereof, (b) a non-coding RNA (e.g., piRNA, miRNA, shRNA, siRNA, antisense RNA) that targets an endogenous mutant form of CFTR, (c) a CRISPR/Cas system that targets an endogenous mutant form of CFTR; and/or (d) any combination of any one of a nucleic acids listed in (a) to (c). In some embodiments, a virion or pharmaceutical composition is delivered to lung via an intranasal or intrapulmonary administration. In some embodiments, a virion or pharmaceutical composition (a) increases expression of CFTR or fragment thereof; and/or (b) decreases expression of an endogenous mutant form of CFTR in a transduced cell. In some embodiments, a virion or pharmaceutical composition prevents or treats cystic fibrosis.

In some embodiments, a virion, a population of virions, a preparation, a construct, composition, or a pharmaceutical composition comprises a nucleic encoding (a) Col4a3 or a fragment thereof, (b) a non-coding RNA (e.g., piRNA, miRNA, shRNA, siRNA, antisense RNA) that targets an endogenous mutant form of Col4a3, (c) a CRISPR/Cas system that targets an endogenous mutant form of Col4a3; and/or (d) any combination of any one of a nucleic acids listed in (a) to (c). In some embodiments, a virion or pharmaceutical composition is delivered to kidney via systemic administration. In some embodiments, a virion, composition, or pharmaceutical composition (a) increases expression of Col4a3 or fragment thereof; and/or (b) decreases expression of an endogenous mutant form of Col4a3 in a transduced cell. In some embodiments, a virion or pharmaceutical composition prevents or treats Alport syndrome.

In some embodiments, a virion, a population of virions, a preparation, a construct, composition, or a pharmaceutical composition comprises a nucleic encoding (a) Col4a4 or a fragment thereof, (b) a non-coding RNA (e.g., piRNA, miRNA, shRNA, siRNA, antisense RNA) that targets an endogenous mutant form of Col4a4, (c) a CRISPR/Cas system that targets an endogenous mutant form of Col4a4; and/or (d) any combination of any one of a nucleic acids listed in (a) to (c). In some embodiments, a virion or pharmaceutical composition is delivered to kidney via systemic administration. In some embodiments, a virion, composition, or pharmaceutical composition (a) increases expression of Col4a4 or fragment thereof; and/or (b) decreases expression of an endogenous mutant form of Col4a4 in a transduced cell. In some embodiments, a virion or pharmaceutical composition prevents or treats Alport syndrome.

In some embodiments, a virion, a population of virions, a preparation, a construct, composition, or a pharmaceutical composition comprises a nucleic encoding (a) Col4a5 or a fragment thereof, (b) a non-coding RNA (e.g., piRNA, miRNA, shRNA, siRNA, antisense RNA) that targets an endogenous mutant form of Col4a5, (c) a CRISPR/Cas system that targets an endogenous mutant form of Col4a5; and/or (d) any combination of any one of a nucleic acids listed in (a) to (c). In some embodiments, a virion or pharmaceutical composition is delivered to kidney via systemic administration. In some embodiments, a virion, composition, or pharmaceutical composition (a) increases expression of Col4a5 or fragment thereof; and/or (b) decreases expression of an endogenous mutant form of Col4a5 in a transduced cell. In some embodiments, a virion or pharmaceutical composition prevents or treats Alport syndrome.

In some embodiments, a virion, a population of virions, a preparation, a construct, composition, or a pharmaceutical composition comprises a nucleic encoding (a) GLA or a fragment thereof, (b) a non-coding RNA (e.g., piRNA, miRNA, shRNA, siRNA, antisense RNA) that targets an endogenous mutant form of GLA, (c) a CRISPR/Cas system that targets an endogenous mutant form of GLA; and/or (d) any combination of any one of a nucleic acids listed in (a) to (c). In some embodiments, a virion or pharmaceutical composition is delivered to kidney via systemic administration. In some embodiments, a virion, composition, or pharmaceutical composition (a) increases expression of GLA or fragment thereof; and/or (b) decreases expression of an endogenous mutant form of GLA in a transduced cell. In some embodiments, a virion or pharmaceutical composition prevents or treats Fabry disease.

In some embodiments, a virion, a population of virions, a preparation, a construct, composition, or a pharmaceutical composition comprises a nucleic encoding (a) PKD1 or a fragment thereof, (b) a non-coding RNA (e.g., piRNA, miRNA, shRNA, siRNA, antisense RNA) that targets an endogenous mutant form of PKD1, (c) a CRISPR/Cas system that targets an endogenous mutant form of PKD1; and/or (d) any combination of any one of a nucleic acids listed in (a) to (c). In some embodiments, a virion or pharmaceutical composition is delivered to kidney via systemic administration. In some embodiments, a virion, composition, or pharmaceutical composition (a) increases expression of PKD1 or fragment thereof; and/or (b) decreases expression of an endogenous mutant form of PKD1 in a transduced cell. In some embodiments, a virion or pharmaceutical composition prevents or treats autosomal dominant polycystic kidney disease (PKD).

In some embodiments, a virion, a population of virions, a preparation, a construct, composition, or a pharmaceutical composition comprises a nucleic encoding (a) PKD2 or a fragment thereof, (b) a non-coding RNA (e.g., piRNA, miRNA, shRNA, siRNA, antisense RNA) that targets an endogenous mutant form of PKD2, (c) a CRISPR/Cas system that targets an endogenous mutant form of PKD2; and/or (d) any combination of any one of a nucleic acids listed in (a) to (c). In some embodiments, a virion or pharmaceutical composition is delivered to kidney via systemic administration. In some embodiments, a virion, composition, or pharmaceutical composition (a) increases expression of PKD2 or fragment thereof; and/or (b) decreases expression of an endogenous mutant form of PKD2 in a transduced cell. In some embodiments, a virion or pharmaceutical composition prevents or treats autosomal dominant polycystic kidney disease (PKD).

In some embodiments, a virion, a population of virions, a preparation, a construct, composition, or a pharmaceutical composition comprises a nucleic encoding (a) NPHS1 (nephrin) or a fragment thereof, (b) a non-coding RNA (e.g., piRNA, miRNA, shRNA, siRNA, antisense RNA) that targets an endogenous mutant form of NPHS1, (c) a CRISPR/Cas system that targets an endogenous mutant form of NPHS1; and/or (d) any combination of any one of a nucleic acids listed in (a) to (c). In some embodiments, a virion or pharmaceutical composition is delivered to kidney via systemic administration. In some embodiments, a virion, composition, or pharmaceutical composition (a) increases expression of NPHS1 or fragment thereof; and/or (b) decreases expression of an endogenous mutant form of NPHS1 in a transduced cell. In some embodiments, a virion or pharmaceutical composition prevents or treats congenital nephrotic syndrome.

In some embodiments, a virion, a population of virions, a preparation, a construct, composition, or a pharmaceutical composition comprises a nucleic encoding (a) NPHS2 (podocin) or a fragment thereof, (b) a non-coding RNA (e.g., piRNA, miRNA, shRNA, siRNA, antisense RNA) that targets an endogenous mutant form of NPHS2, (c) a CRISPR/Cas system that targets an endogenous mutant form of NPHS2; and/or (d) any combination of any one of a nucleic acids listed in (a) to (c). In some embodiments, a virion or pharmaceutical composition is delivered to kidney via systemic administration. In some embodiments, a virion, composition, or pharmaceutical composition (a) increases expression of NPHS2 or fragment thereof; and/or (b) decreases expression of an endogenous mutant form of NPHS2 in a transduced cell. In some embodiments, a virion or pharmaceutical composition prevents or treats congenital nephrotic syndrome.

In some embodiments, a virion, a population of virions, a preparation, a construct, composition, or a pharmaceutical composition comprises a nucleic encoding (a) MYBPC3 or a fragment thereof, (b) a non-coding RNA (e.g., piRNA, miRNA, shRNA, siRNA, antisense RNA) that targets an endogenous mutant form of MYBPC3, (c) a CRISPR/Cas system that targets an endogenous mutant form of MYBPC3; and/or (d) any combination of any one of a nucleic acids listed in (a) to (c). In some embodiments, a virion or pharmaceutical composition is delivered to kidney via systemic administration. In some embodiments, a virion, composition, or pharmaceutical composition (a) increases expression of MYBPC3 or fragment thereof; and/or (b) decreases expression of an endogenous mutant form of MYBPC3 in a transduced cell. In some embodiments, a virion or pharmaceutical composition prevents or treats hypertrophic cardiomyopathy.

In some embodiments, a virion, a population of virions, a preparation, a construct, composition, or a pharmaceutical composition comprises a nucleic encoding (a) JPH2 or a fragment thereof, (b) a non-coding RNA (e.g., piRNA, miRNA, shRNA, siRNA, antisense RNA) that targets an endogenous mutant form of JPH2, (c) a CRISPR/Cas system that targets an endogenous mutant form of JPH2; and/or (d) any combination of any one of a nucleic acids listed in (a) to (c). In some embodiments, a virion or pharmaceutical composition is delivered to kidney via systemic administration. In some embodiments, a virion, composition, or pharmaceutical composition (a) increases expression of JPH2 or fragment thereof; and/or (b) decreases expression of an endogenous mutant form of JPH2 in a transduced cell. In some embodiments, a virion or pharmaceutical composition prevents or treats hypertrophic cardiomyopathy.

In some embodiments, a virion, a population of virions, a preparation, a construct, composition, or a pharmaceutical composition comprises a nucleic encoding (a) ALPK3 or a fragment thereof, (b) a non-coding RNA (e.g., piRNA, miRNA, shRNA, siRNA, antisense RNA) that targets an endogenous mutant form of ALPK3, (c) a CRISPR/Cas system that targets an endogenous mutant form of ALPK3; and/or (d) any combination of any one of a nucleic acids listed in (a) to (c). In some embodiments, a virion or pharmaceutical composition is delivered to kidney via systemic administration. In some embodiments, a virion, composition, or pharmaceutical composition (a) increases expression of ALPK3 or fragment thereof; and/or (b) decreases expression of an endogenous mutant form of ALPK3 in a transduced cell. In some embodiments, a virion or pharmaceutical composition prevents or treats hypertrophic cardiomyopathy.

In some embodiments, a virion, a population of virions, a preparation, a construct, composition, or a pharmaceutical composition comprises a nucleic encoding (a) ALPK3 or a fragment thereof, (b) a non-coding RNA (e.g., piRNA, miRNA, shRNA, siRNA, antisense RNA) that targets an endogenous mutant form of ALPK3, (c) a CRISPR/Cas system that targets an endogenous mutant form of ALPK3; and/or (d) any combination of any one of a nucleic acids listed in (a) to (c). In some embodiments, a virion or pharmaceutical composition is delivered to kidney via systemic administration. In some embodiments, a virion, composition, or pharmaceutical composition (a) increases expression of ALPK3 or fragment thereof; and/or (b) decreases expression of an endogenous mutant form of ALPK3 in a transduced cell. In some embodiments, a virion or pharmaceutical composition prevents or treats dilated cardiomyopathy.

In some embodiments, a virion, a population of virions, a preparation, a construct, composition, or a pharmaceutical composition comprises a nucleic encoding (a) RBM20 or a fragment thereof, (b) a non-coding RNA (e.g., piRNA, miRNA, shRNA, siRNA, antisense RNA) that targets an endogenous mutant form of RBM20, (c) a CRISPR/Cas system that targets an endogenous mutant form of RBM20; and/or (d) any combination of any one of a nucleic acids listed in (a) to (c). In some embodiments, a virion or pharmaceutical composition is delivered to kidney via systemic administration. In some embodiments, a virion, composition, or pharmaceutical composition (a) increases expression of RBM20 or fragment thereof; and/or (b) decreases expression of an endogenous mutant form of RBM20 in a transduced cell. In some embodiments, a virion or pharmaceutical composition prevents or treats dilated cardiomyopathy.

In some embodiments, a virion, a population of virions, a preparation, a construct, composition, or a pharmaceutical composition comprises a nucleic encoding (a) PKP2 or a fragment thereof, (b) a non-coding RNA (e.g., piRNA, miRNA, shRNA, siRNA, antisense RNA) that targets an endogenous mutant form of PKP2, (c) a CRISPR/Cas system that targets an endogenous mutant form of PKP2; and/or (d) any combination of any one of a nucleic acids listed in (a) to (c). In some embodiments, a virion or pharmaceutical composition is delivered to kidney via systemic administration. In some embodiments, a virion, composition, or pharmaceutical composition (a) increases expression of PKP2 or fragment thereof; and/or (b) decreases expression of an endogenous mutant form of PKP2 in a transduced cell. In some embodiments, a virion or pharmaceutical composition prevents or treats dilated cardiomyopathy.

In some embodiments, a virion, a population of virions, a preparation, a construct, composition, or a pharmaceutical composition comprises a nucleic encoding (a) LMNA or a fragment thereof, (b) a non-coding RNA (e.g., piRNA, miRNA, shRNA, siRNA, antisense RNA) that targets an endogenous mutant form of LMNA, (c) a CRISPR/Cas system that targets an endogenous mutant form of LMNA; and/or (d) any combination of any one of a nucleic acids listed in (a) to (c). In some embodiments, a virion or pharmaceutical composition is delivered to kidney via systemic administration. In some embodiments, a virion, composition, or pharmaceutical composition (a) increases expression of LMNA or fragment thereof; and/or (b) decreases expression of an endogenous mutant form of LMNA in a transduced cell. In some embodiments, a virion or pharmaceutical composition prevents or treats dilated cardiomyopathy.

In some embodiments, a virion, a population of virions, a preparation, a construct, composition, or a pharmaceutical composition comprises a nucleic encoding (a) BAG3 or a fragment thereof, (b) a non-coding RNA (e.g., piRNA, miRNA, shRNA, siRNA, antisense RNA) that targets an endogenous mutant form of BAG3, (c) a CRISPR/Cas system that targets an endogenous mutant form of BAG3; and/or (d) any combination of any one of a nucleic acids listed in (a) to (c). In some embodiments, a virion or pharmaceutical composition is delivered to kidney via systemic administration. In some embodiments, a virion, composition, or pharmaceutical composition (a) increases expression of BAG3 or fragment thereof; and/or (b) decreases expression of an endogenous mutant form of BAG3 in a transduced cell. In some embodiments, a virion or pharmaceutical composition prevents or treats dilated cardiomyopathy.

In some embodiments, methods of preventing or treating a disease further include re-administering an additional amount of a virion, population of virions, preparation, composition, pharmaceutical composition, or transduced cells. In some embodiments, the re-administering an additional amount is performed after an attenuation in a treatment subsequent to administering an initial effective amount of a virion, pharmaceutical composition, or transduced cells. In some embodiments, an additional amount is the same as an initial effective amount. In some embodiments, an additional amount is more than an initial effective amount. In some embodiments, an additional amount is less than an initial effective amount. In certain embodiments, an additional amount is increased or decreased based on expression of an endogenous gene and/or a nucleic acid of a virion. An endogenous gene includes a biomarker gene whose expression is, e.g., indicative of or relevant to diagnosis and/or prognosis of a disease.

In some embodiments, methods of preventing or treating a disease further comprise administering to a subject or contacting cells with an agent that modulates expression of a nucleic acid. In some embodiments, an agent is selected from a small molecule, a metabolite, an oligonucleotide, a riboswitch, a peptide, a peptidomimetic, a hormone, a hormone analog, and light. In some embodiments, an agent is selected from tetracycline, cumate, tamoxifen, estrogen, and an antisense oligonucleotide (ASO). In some embodiments, methods further comprise re-administering an agent one or more times at intervals. In some embodiments, re-administration of an agent results in pulsatile expression of a nucleic acid. In some embodiments, time between the intervals and/or amount of an agent is increased or decreased based on serum concentration and/or half-life of a protein expressed from a nucleic acid.

In some embodiments, further provided herein are methods of modulating (i) gene expression, or (ii) function and/or structure of a protein in a cell, the method comprising transducing a cell with a virion or pharmaceutical composition described herein comprising a nucleic acid that modulates gene expression, or function and/or structure of a protein in a cell. In some embodiments, such nucleic acid comprises a sequence encoding CRISPRi or CRISPRa agents. In some embodiments, gene expression, or function and/or structure of a protein is increased or restored. In some embodiments, gene expression, or function and/or structure of a protein is decreased or eliminated.

c. Methods of Delivering a Transgene to a Genomic Safe Harbor (GSH)

Among other things, in some embodiments, the present disclosure provides for a method of delivering a transgene to a genomic safe harbor (GSH).

Genomic safe harbors (GSH) are intragenic, intergenic, or extragenic regions of the human and model species genomes that are able to accommodate the predictable expression of newly integrated DNA without significant adverse effects on the host cell or organism. GSHs may comprise intronic or exonic gene sequences as well as intergenic or extragenic sequences. While not being limited to theory, a useful safe harbor must permit sufficient transgene expression to yield desired levels of the transgene-encoded protein or non-coding RNA. A GSH also should not predispose cells to malignant transformation, nor interfere with progenitor cell differentiation, nor significantly alter normal cellular functions. What distinguishes a GSH from a fortuitous good integration event is the predictability of outcome, which is based on prior knowledge and validation of a GSH.

The larger genome size of a virion described herein allows delivery of a therapeutic transgene(s) together with GSH sequences, which is otherwise not possible with virions having a limited genome size, e.g., AAV. Accordingly, virions of the present disclosure not only facilitates delivery of a larger transgene compared with e.g., AAV, but also facilitates a safe delivery of a transgene by allowing codelivery of a GSH sequences that ensures predictable expression of a transgene without adverse effects on host cells. Exemplary GSHs that have been targeted for transgene addition include (i) the adeno-associated virus site 1 (AAVS1), a naturally occurring, non-germline, site of integration of AAV virus DNA on chromosome 19; (ii) chemokine (C-C motif) receptor 5 (CCR5) gene, a chemokine receptor gene known as an HIV-1 coreceptor; (iii) human ortholog of the mouse Rosa26 locus, a locus extensively validated in the murine setting for the insertion of ubiquitously expressed transgenes; (iii) a T cell receptor locus (TCR), such as TCR alpha or TCR beta, and (iv) albumin in murine cells (see, e.g., U.S. Pat. Nos. 7,951,925; 8,771,985; 8,110,379; and 7,951,925; U.S. Patent Publication Nos. 2010/0218264; 2011/0265198; 2013/0137104; 2013/0122591; 2013/0177983; 2013/0177960; 2015/0056705 and 2015/0159172; all of which are incorporated by reference). Additional GSHs include Kif6, Pax5, collagen, HTRP, HI 1 (a thymidine kinase encoding nucleic acid at HI 1 locus), beta-2 microglobulin, GAPDH, TCR, RUNX1, KLHL7, NUPL2 or an intergenic region thereof, mir684, KCNH2, GPNMB, MIR4540, MIR4475, MIR4476, PRL32P21, LOC105376031, LOC105376032, LOC105376030, MELK, EBLN3P, ZCCHC7, RNF38, or loci meeting the criteria of a genome safe harbor as described herein (see e.g., WO 2019/169233 A1, WO 2017/079673 A1; incorporated by reference). GSHs described herein provide a non-limiting representation of GSHs that can be used with virions described herein. The present disclosure contemplates use of any GSHs that are known in the art.

In some embodiments, a GSH allows safe and targeted gene delivery that has limited off-target activity and minimal risk of genotoxicity, or causing insertional oncogenesis upon integration of foreign DNA, while being accessible to highly specific nucleases with minimal off-target activity.

In some embodiments, a GSH has any one or more of the following properties: (i) outside a gene transcription unit; (ii) located between 5-50 kilobases (kb) away from the 5′ end of any gene; (iii) located between 5-300 kb away from cancer-related genes; (iv) located 5-300 kb away from any identified microRNA; and (v) outside ultra-conserved regions and long noncoding RNAs. In some embodiments, a GSH locus has any or more of the following properties: (i) outside a gene transcription unit; (ii) located >50 kilobases (kb) from the 5′ end of any gene; (iii) located >300 kb from cancer-related genes; (iv) located >300 kb from any identified microRNA; and (v) outside ultra-conserved regions and long noncoding RNAs. In studies of lentiviral construct integrations in transduced induced pluripotent stem cells, analysis of over 5,000 integration sites revealed that-17% of integrations occurred in safe harbors. Virions that integrated into these safe harbors were able to express therapeutic levels of β-globin from their transgene without perturbing endogenous gene expression.

In some embodiments, a GSH is AAVS1. AAVS1 was identified as the adeno-associated virus common integration site on chromosome 19 and is located in chromosome 19 (position 19ql3.42) and was primarily identified as a repeatedly recovered site of integration of wild-type AAV in the genome of cultured human cell lines that have been infected with AAV in vitro. Integration in the AAVS1 locus interrupts the gene phosphatase 1 regulatory subunit 12C (PPP1R12C; also known as MBS85), which encodes a protein with a function that is not clearly delineated. The organismal consequences of disrupting one or both alleles of PPP1R12C are currently unknown. No gross abnormalities or differentiation deficits were observed in human and mouse pluripotent stem cells harboring transgenes targeted in AAVS1. Originally, AAV DNA integration into AAVS1 site was Rep-dependent, however, there are commercially available CRISPR/Cas9 reagents available for targeting which preserved the functionality of the targeted allele and maintained the expression of PPP1R12C at levels that are comparable to those in non-targeted cells. AAVS1 was also assessed using ZFN-mediated recombination into iPSCs or CD34+ cells.

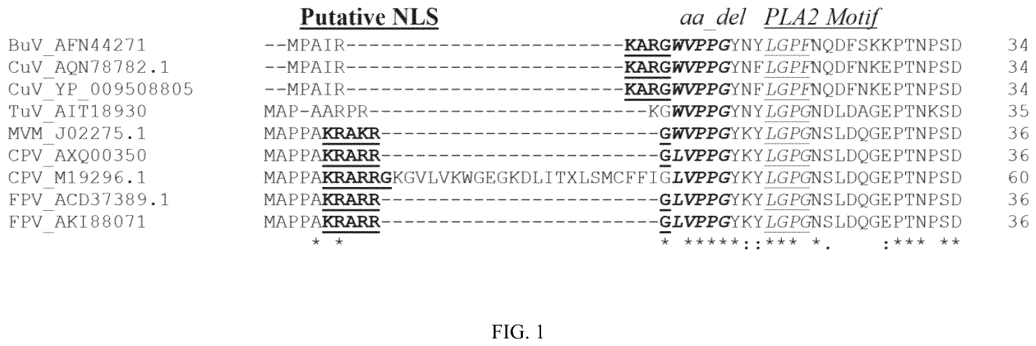

As originally characterized, the AAVS1 locus is >4 kb and is identified as chromosome 19 nucleotides 55,113,873-55,117,983 (human genome assembly GRCh38/hg38) and overlaps with exon 1 of the PPP1R12C gene that encodes protein phosphatase 1 regulatory subunit 12C. This >4 kb region is extremely G+C nucleotide content rich and is a gene-rich region of particularly gene-rich chromosome 19 (see FIG. 1A of Sadelain et al, Nature Revs Cancer, 2012; 12; 51-58), and some integrated promoters can indeed activate or cis-activate neighboring genes, the consequence of which in different tissues is presently unknown. PPP1R12C exon 1 5′untranslated region contains a functional AAV origin of DNA synthesis indicated within a known sequence (Urcelay et al. 1995).

AAVS1 GSH was identified by characterizing an AAV provirus structure in latently infected human cell lines with recombinant bacteriophage genomic libraries generated from latently infected clonal cell lines (Detroit 6 clone 7374 IIID5) (Kotin and Berns 1989), Kotin et al, isolated non-viral, cellular DNA flanking the provirus and used a subset of “left” and “right” flanking DNA fragments as probes to screen panels of independently derived latently infected clonal cell lines. In approximately 70% of the clonal isolates, AAV DNA was detected with the cell-specific probe (Kotin et al. 1991; Kotin et al. 1990). Sequence analysis of the pre-integration site identified near homology to a portion of the AAV inverted terminal repeat (Kotin, Linden, and Beerns 1992). Although lacking the characteristic interrupted palindrome, the AAVS1 locus retained the Rep binding elements and terminal resolution sites homologous to the AAV ITR.

Selection of the exonic integration site is non-obvious, and perhaps counter-intuitive, since insertion and expression of foreign DNA likely disrupts expression of endogenous genes. Apparently, insertion of an AAV genome into this locus does not adversely affect cell viability or iPSC differentiation (DeKelver et al. 2010; Wang et al. 2012; Zou et al. 2011). AAVS1 locus is within a 5′ UTR of the highly conserved PPP1R12C gene. The Rep-dependent minimal origin of DNA synthesis is conserved in a 5′UTR of a human, chimapanzee, and gorilla PPP1R12C gene. However, commercially available CRISPR/Cas9 reagents used for integrating DNA into AAVS1 target PPP1R12C intron 1 rather than an exon.

In some embodiments, a GSH is any one of Kif6, Pax5, collagen, HTRP, HI 1, beta-2 microglobulin, GAPDH, TCR, RUNX1, KLHL7, an intergenic region of NUPL2, mir684, KCNH2, GPNMB, MIR4540, MIR4475, MIR4476, PRL32P21, LOC105376031, LOC105376032, LOC105376030, MELK, EBLN3P, ZCCHC7, and RNF38.

In some embodiments, a GSH is a Pax 5 gene (also known as Paired Box 5, or “B-cell lineage specific activator protein,” or BSAP). In humans PAX5 is located on chromosome 9 at 9p 13.2 and has orthologues across many vertebrate species, including, human, chimp, macaque, mouse, rat, dog, horse, cow, pig, opossum, platypus, chicken, lizard, xenopus, C. elegans, drosophila and zebrafish. PAX5 gene is located at Chromosome 9:36, 833,275-37,034, 185 reverse strand (GRCh38: CM000671.2) or 36,833,272-37,034,182 in GRCh37 coordinates.

Additional exemplary GSHs are listed in Table 5A and Table 5B.

TABLE 5A
Exemplary GSH loci in Homo Sapiens (see, e.g.,
WO 2019/169232; incorporated by reference)

| Gene | Chromosomal location | Accession number/location |
|---|---|---|
| PAX5 | Chromosome 9: 36,833,275- | NC_000009.12 (36833274 . . . 37035949, |
| | 37,034,185 reverse strand | complement) |
| MIR4540 | — | NC_000009.12 (36864254 . . . 36864308, |
| | | complement) |
| MIR4475 | GRCh38.p7 | NC_000009.12 (36823539 . . . 36823599, |
| | (GCF_000001405.33) | complement) |
| MIR4476 | GRCh38.p7 | NC_000009.12 (36893462 . . . 36893531, |
| | (GCF_000001405.33) | complement) |
| PRL32P21 | GRCh38.p7 | NC_000009.12 (37046835 . . . 37047242) |
| | (GCF_000001405.33) | |
| LOC105376031 | GRCh38.p7 | NC_000009.12 (37027763 . . . 37031333) |
| | (GCF_000001405.33) | |
| LOC105376032 | GRCh38.p7 | NC_000009.12 (37002697 . . . 37007774) |
| | (GCF_000001405.33) | |
| LOC105376030 | GRCh38.p7 | NC_000009.12 (36779475 . . . 36830456) |
| | (GCF_000001405.33) | |
| MELK | GRCh38.p7 | NC_000009.12 (36572862 . . . 36677683) |
| | (GCF_000001405.33) | |
| EBLN3P | GRCh38.p7 | NC_000009.12 (37079896 . . . 37090401) |
| | (GCF_000001405.33) | |
| ZCCHC7 | GRCh38.p7 | NC_000009.12 (37120169 . . . 37358149) |
| | (GCF_000001405.33) | |
| RNF38 | GRCh38.p7 | NC_000009.12 (36336398 . . . 36487384, |
| | (GCF_000001405.33) | complement) |

TABLE 5B
Exemplary GSH loci (see, e.g., WO 2019/169232; incorporated by reference)

| | | | |
|---|---|---|---|
| Taxonomic | | | |
| Rank | Brief description | Species | Chromosomal location |

[symbol]

loci

| | | | |
|---|---|---|---|
| [symbol] | mAAV_eve integration between | M. domestica | chromosome [symbol] |
| (taxonomic | cadherin [symbol] and [symbol] 16. | | [symbol] |
| rank: Family) | Because the [symbol] genome is | | [symbol] |
| | [symbol] another | | Intergenic distance: [symbol] |
| | [symbol] | | Empty EVE locus in M. domestica 674,422,470-675,422,729 |
| | with a more completely assemble | | |
| | genome is used as a substitute | Mouse | [symbol] |
| | genome. | | [symbol] |
| | | | cdh 11: [symbol] |
| | | | Intergenic distance = 3.2 Mb |
| | | Homo sapiens | Chromosome 16 |
| | | | cdh 8: [symbol] |
| | | | cdh 11: [symbol] |
| | | | Intergenic distance: 2.9 Mb |
| [symbol] | [symbol] EVE located between | H. Sapiens | Chromosome [symbol] |
| (Family) - the | [symbol] and [symbol] | | [symbol] |
| Family | The gene order is: | M. mus | [symbol] |
| [symbol] are | [symbol] | | |
| rabbits and | [symbol] | | |
| [symbol] species | [symbol] | | |
| of the | | | |
| [symbol] | | | |
| Order. | | | |

[symbol]

loci

| | | | |
|---|---|---|---|
| Cetacea | EVE integrated into an [symbol] of | H. sapiens | chromosome [symbol] |
| (Order) | PAX [symbol] | | [symbol] |
| | | M. mus | Chromosome [symbol] |
| | | | [symbol] |
| (Family- | [symbol] EVE integrated into the [symbol] | H. sapiens | Chromosome 6 |
| [symbol] | gene, [symbol] | | [symbol] |
| Order - | | | Chromosome 17 |
| [symbol] | | | [symbol] |
| [symbol] | | | |
| (Genus), | | | |
| [symbol] | | | |
| (Subfamily) | | | |

[symbol]

indicates data missing or illegible when filed

d. Methods of Integration into a Target Genome

Among other things, in some embodiments, the present disclosure provides for a method of integration into a target genome.

Integration to a target genome may be driven by cellular processes, such as homologous recombination or non-homologous end-joining (NHEJ). Integration may also be initiated and/or facilitated by an exogenously introduced nuclease. In preferred embodiments, a nucleic acid packaged within a virion described herein is integrated to a specific locus within a genome, e.g., a GSH. In some embodiments, a GSH is any locus that permits sufficient transgene expression to yield desired levels of the transgene-encoded protein or non-coding RNA. A GSH also should not predispose cells to malignant transformation nor significantly alter normal cellular functions. Site-specific integration to a GSH may be mediated by a nucleic acid homologous to a GSH that is placed 5′ and 3′ to a nucleic acid to be integrated. Such homologous donor sequences may provide a template for homology-dependent repair that allows integration at the desired locus.

In preferred embodiments, a virion described herein comprises a nucleic acid comprising a nucleic acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a nucleic acid sequence of a genomic safe harbor (GSH) of a target cell. In some embodiments, said nucleic acid that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a GSH is placed 5′ and 3′ (homology arms) to a nucleic acid to be integrated, thereby allowing insertion (of a nucleic acid located between homology arms) to a specific locus in a target genome by homologous recombination. In some embodiments, a nucleic acid to be integrated is any one of a nucleic acids operably linked to a promoter described herein. In some embodiments, a GSH is AAVS1, ROSA26, CCR5, Kif6, Pax5, an intergenic region of NUPL2, collagen, HTRP, HI 1 (a thymidine kinase encoding nucleic acid at HI 1 locus), beta-2 microglobulin, GAPDH, TCR, RUNX1, KLHL7, mir684, KCNH2, GPNMB, MIR4540, MIR4475, MIR4476, PRL32P21, LOC105376031, LOC105376032, LOC105376030, MELK, EBLN3P, ZCCHC7, or RNF38. In some embodiments, a GSH is AAVS1, ROSA26, CCR5, Kif6, Pax5, or an intergenic region of NUPL2.

In certain embodiments, a coding sequence of a virion is integrated into a genome of a target cell upon transduction. In some embodiments, a nucleic acid is integrated into a GSH or EVE. In some embodiments, a GSH is AAVS1, ROSA26, CCR5, Kif6, Pax5, an intergenic region of NUPL2, collagen, HTRP, HI 1 (a thymidine kinase encoding nucleic acid at HI 1 locus), beta-2 microglobulin, GAPDH, TCR, RUNX1, KLHL7, mir684, KCNH2, GPNMB, MIR4540, MIR4475, MIR4476, PRL32P21, LOC105376031, LOC105376032, LOC105376030, MELK, EBLN3P, ZCCHC7, or RNF38. In some embodiments, a GSH is AAVS1, ROSA26, CCR5, Kif6, Pax5, or an intergenic region of NUPL2. In some embodiments, a nucleic acid is integrated into a target genome by homologous recombination followed by a DNA break formation induced by an exogenously-introduced nuclease. In some embodiments, a nuclease is TALEN, ZEN, a meganuclease, a megaTAL, or a CRISPR endonuclease (e.g., a Cas9 endonuclease or a variant thereof). In some embodiments, a CRISPR endonuclease is in a complex with a guide RNA.

In some embodiments, provided herein are methods of integrating a heterologous nucleic acid into a GSH in a cell, comprising: (a) transducing a cell with one or more virions described herein comprising a heterologous nucleic acid flanked at the 5′ end and 3′ end by a donor nucleic acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the target GSH nucleic acid; or (b) transducing the cell with one or more virions described herein comprising (i) a heterologous nucleic acid flanked at a 5′ end and 3′ end by a donor nucleic acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the target GSH nucleic acid, and (ii) a nucleic acid encoding a nuclease (e.g., Cas9 or a variant thereof, ZFN, TALEN) and/or a guide RNA, wherein a nuclease or the nuclease/gRNA complex makes a DNA break at a GSH, which is repaired using a donor nucleic acid, thereby integrating a heterologous nucleic acid at GSH. In some embodiments, (i) a heterologous nucleic acid flanked by a donor nucleic acid that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a target GSH nucleic acid and (ii) a nucleic acid encoding a nuclease and/or the gRNA are transduced in separate virions. In some embodiments, a GSH is AAVS1, ROSA26, CCR5, Kif6, Pax5, an intergenic region of NUPL2, collagen, HTRP, HI 1 (a thymidine kinase encoding nucleic acid at HI 1 locus), beta-2 microglobulin, GAPDH, TCR, RUNX1, KLHL7, mir684, KCNH2, GPNMB, MIR4540, MIR4475, MIR4476, PRL32P21, LOC105376031, LOC105376032, LOC105376030, MELK, EBLN3P, ZCCHC7, or RNF38. In some embodiments, a GSH is AAVS1, ROSA26, CCR5, Kif6, Pax5, or an intergenic region of NUPL2.

For integration of a nucleic acid located between the 5′ and 3′ homology arms, the 5′ and 3′ homology arms should be long enough for targeting to a GSH and allow (e.g., guide) integration into a genome by homologous recombination. To increase the likelihood of integration at a precise location and enhance probability of homologous recombination, the 5′ and 3′ homology arms may include a sufficient number of nucleic acids. In some embodiments, the 5′ and 3′ homology arms may include at least 10 base pairs but no more than 5,000 base pairs, at least 50 base pairs but no more than 5,000 base pairs, at least 100 base pairs but no more than 5,000 base pairs, at least 200 base pairs but no more than 5,000 base pairs, at least 250 base pairs but no more than 5,000 base pairs, or at least 300 base pairs but no more than 5,000 base pairs. In some embodiments, the 5′ and 3′ homology arms include about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 base pairs. Detailed information regarding length of homology arms and recombination frequency is art-known, see e.g., Zhang et al. “Efficient precise knock in with a double cut HDR donor after CRISPR/Cas9-mediated double-stranded DNA cleavage.” Genome biology 18.1 (2017): 35, which is incorporated herein in its entirety by reference.

5′ and 3′ homology arms may be any sequence that is homologous with a GSH target sequence in a genome of a host cell. In some embodiments, 5′ and 3′ homology arms may be homologous to portions of a GSH described herein. Furthermore, 5′ and 3′ homology arms may be non-coding or coding nucleotide sequences.

In some embodiments, a 5′ and/or 3′ homology arms can be homologous to a sequence immediately upstream and/or downstream of the integration or DNA cleavage site on the chromosome. Alternatively, the 5′ and/or 3′ homology arms can be homologous to a sequence that is distant from the integration or DNA cleavage site, such as at least 1, 2, 5, 10, 15, 20, 25, 30, 50, 75,100,125, 150, 175, 200, 225, 250, 275, 300, 325, 350, 375, 400, 425, 450, 475, 500 or more base pairs away from the integration or DNA cleavage site, or partially or completely overlapping with a DNA cleavage site (e.g., can be a DNA break induced by an exogenously-introduced nuclease). In some embodiments, a 3′ homology arm of the nucleotide sequence is proximal to an ITR.

### 4. Administration

Provided herein are technologies comprising, among other things, therapeutic delivery systems for treating a disease or disorder. In some embodiments the present disclosure provides compositions that are part of or comprise at least one construct, e.g., viral construct, e.g., a protoparvovirus variant VP1 construct. In some such embodiments, a composition comprises a virion. In some embodiments, a virion comprises a protoparvovirus variant VP1 capsid polypeptide.

a. Routes of Administration

In some embodiments, the present disclosure provides various routes of and formulations for administration. As will be known to one of skill in the art, pharmaceutical forms suitable for injectable use include sterile aqueous solutions or dispersions and sterile powders for extemporaneous preparation of sterile injectable solutions or dispersions. Dispersions may also be prepared in glycerol, liquid polyethylene glycols, and mixtures thereof and in oils.

Under ordinary conditions of storage and use, these preparations contain a preservative to prevent growth of microorganisms. In many cases the form is sterile and fluid to the extent that easy syringability exists. It must be stable under conditions of manufacture and storage and must be preserved against contaminating action of microorganisms, such as bacteria and fungi. In some embodiments, a carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by use of a coating, such as lecithin, by maintenance of the required particle size in the case of dispersion and by use of surfactants. Prevention of action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride.

Prolonged absorption of injectable compositions can be brought about use in compositions of agents delaying absorption, for example, aluminum monostearate and gelatin. For administration of an injectable aqueous solution, for example, a solution may be suitably buffered, if necessary, and a liquid diluent first rendered isotonic with sufficient saline or glucose. Aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at a proposed site of infusion, (see for example, “Remington's Pharmaceutical Sciences” 15th Edition, pages 1035-1038 and 1570-1580, which is incorporated in its entirety herein by reference). Some variation in dosage will necessarily occur depending on condition of a host. A person responsible for administration will, in any event, determine an appropriate dose for an individual host.

In some embodiments, sterile injectable solutions are prepared by incorporating active virion in a required amount in an appropriate solvent with various other ingredients enumerated herein, as required, followed by filtered sterilization. Generally, dispersions are prepared by incorporating various sterilized active ingredients into a sterile vehicle which contains basic dispersion medium and required other ingredients from those enumerated above. In the case of sterile powders for preparation of sterile injectable solutions, in some embodiments, preferred methods of preparation are vacuum-drying and freeze-drying techniques which yield a powder of active ingredient plus any additional desired ingredient from a previously sterile-filtered solution thereof.

In some embodiments, virion compositions disclosed herein may also be formulated in a neutral or salt form. Pharmaceutically-acceptable salts, include acid addition salts (formed with free amino groups of a given protein) and which are formed with inorganic acids such as, for example, hydrochloric or phosphoric acids, or such organic acids as acetic, oxalic, tartaric, mandelic, and the like. Salts formed with free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions can be administered in a manner compatible with the dosage formulation and in such amount as is therapeutically effective. Formulations are easily administered in a variety of dosage forms such as injectable solutions, drug-release capsules, and the like.

Delivery vehicles such as liposomes, nanocapsules, microparticles, microspheres, lipid particles, vesicles, and the like, may be used for introduction of compositions of the present disclosure into suitable host cells. In particular, in some embodiments, virion-construct delivered transgenes may be formulated for delivery either encapsulated in a lipid particle, a liposome, a vesicle, a nanosphere, or a nanoparticle or the like.

Such formulations may be preferred for introduction of pharmaceutically acceptable formulations of nucleic acids or virion constructs disclosed herein. Formation and use of liposomes is generally known to those of skill in the art. Recently, liposomes were developed with improved serum stability and circulation half-times (U.S. Pat. No. 5,741,516, which is incorporated in its entirety herein by reference). Further, various methods of liposome and liposome like preparations as potential drug carriers have been described (U.S. Pat. Nos. 5,567,434; 5,552,157; 5,565,213; 5,738,868 and 5,795,587, each of which is incorporated in its entirety herein by reference).

Liposomes have been used successfully with a number of cell types that are normally resistant to transfection by other procedures. In addition, liposomes are free of DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining efficacy of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 Tm. Sonication of MLVs results in formation of small unilamellar vesicles (SUVs) with diameters in a range of approximately 200 to 500.ANG., containing an aqueous solution in the core.

Alternatively, nanocapsule formulations of a virion may be used. Nanocapsules can generally entrap substances in a stable and reproducible way. To avoid side effects due to intracellular polymeric overloading, such ultrafine particles (sized around 0.1 Tm) should be designed using polymers able to be degraded in vivo. Biodegradable polyalkyl-cyanoacrylate nanoparticles that meet these requirements are contemplated for use.

In addition to methods of delivery described above, the following techniques are also contemplated as alternative methods of delivering a virion to a host. Sonophoresis (i.e., ultrasound) has been used and described in U.S. Pat. No. 5,656,016, which is incorporated in its entirety herein by reference, as a device for enhancing the rate and efficacy of drug permeation into and through a circulatory system. Other drug delivery alternatives contemplated are intraosseous injection (U.S. Pat. No. 5,779,708, which is incorporated in its entirety herein by reference), microchip devices (U.S. Pat. No. 5,797,898, which is incorporated in its entirety herein by reference), ophthalmic formulations (Bourlais et al., 1998, which is incorporated in its entirety herein by reference), transdermal matrices (U.S. Pat. Nos. 5,770,219 and 5,783,208, each of which is incorporated in its entirety herein by reference) and feedback-controlled delivery (U.S. Pat. No. 5,697,899, which is incorporated in its entirety herein by reference).

In some embodiments, administration of any compositions of the present disclosure may be carried out in any convenient manner, including by aerosol inhalation, injection, ingestion, transfusion, implantation or transplantation. Compositions described herein may be administered to a patient trans arterially, subcutaneously, intradermally, intranodally, intramedullary, intramuscularly, by intravenous (i.v.) injection, or intraperitoneally. In some embodiments, a nucleic acid composition of the present disclosure is administered to a patient by intradermal or subcutaneous injection. In some embodiments, a nucleic composition of the present disclosure is administered by i.v. injection.

b. Dosing

In some embodiments, any of the methods disclosed herein comprise a dose-escalation study to assess safety and tolerability in subjects, e.g., mammals, e.g., humans, e.g., patients, with a disease described herein. In some embodiments, a preparation, a construct(s), a virion, a population of virions, a composition, or a pharmaceutical composition disclosed herein is administered at a dosing regimen disclosed herein. In some embodiments, the dosing regimen comprises either unilateral or bilateral intracochlear administrations of a dose, e.g., as described herein, of a preparation, a construct(s), a virion, a population of virions, a composition, or a pharmaceutical composition disclosed herein. In some embodiments, a dosing regimen comprises delivery in a volume of at least 0.001 mL, 0.005 mL, 0.01 mL, at least 0.02 mL, at least 0.03 mL, at least 0.04 mL, at least 0.05 mL, at least 0.06 mL, at least 0.07 mL, at least 0.08 mL, at least 0.09 mL, at least 0.10 mL, at least 0.11 mL, at least 0.12 mL, at least 0.13 mL, at least 0.14 mL, at least 0.15 mL, at least 0.16 mL, at least 0.17 mL, at least 0.18 mL, at least 0.19 mL, or at least 0.20 mL per cochlea. In some embodiments, the dosing regimen comprises delivery in a volume of at most 0.30 mL, at most 0.25 mL, at most 0.20 mL, at most 0.15 mL, at most 0.14 mL, at most 0.13 mL, at most 0.12 mL, at most 0.11 mL, at most 0.10 mL, at most 0.09 mL, at most 0.08 mL, at most 0.07 mL, at most 0.06 mL, at most 0.05 mL, at most 0.01 mL, at most 0.005 mL, or at most 0.001 mL per cochlea. In some embodiments, the dosing regimen comprises delivery in a volume of about 0.001 mL, 0.005 mL, 0.01 mL, 0.05 mL, about 0.06 mL, about 0.07 mL, about 0.08 mL, about 0.09 mL, about 0.10 mL, about 0.11 mL, about 0.12 mL, about 0.13 mL, about 0.14 mL, or about 0.15 mL per cochlea, depending on the population. In some embodiments, the dosing regimen comprises delivery in a volume of at least 0.001 mL, 0.005 mL, 0.01 mL, at least 0.02 mL, at least 0.03 mL, at least 0.04 mL, at least 0.05 mL, at least 0.06 mL, at least 0.07 mL, at least 0.08 mL, at least 0.09 mL, at least 0.10 mL, at least 0.11 mL, at least 0.12 mL, at least 0.13 mL, at least 0.14 mL, at least 0.15 mL, at least 0.16 mL, at least 0.17 mL, at least 0.18 mL, at least 0.19 mL, or at least 0.20 mL per cochlea. In some embodiments, the dosing regimen comprises delivery in a volume of at most 0.30 mL, at most 0.25 mL, at most 0.20 mL, at most 0.15 mL, at most 0.14 mL, at most 0.13 mL, at most 0.12 mL, at most 0.11 mL, at most 0.10 mL, at most 0.09 mL, at most 0.08 mL, at most 0.07 mL, at most 0.06 mL, at most 0.05 mL, at most 0.01 mL, at most 0.005 mL, or at most 0.001 mL per cochlea. In some embodiments, the dosing regimen comprises delivery in a volume of about 0.001 mL, about 0.005 mL, about 0.01 mL, 0.05 mL, about 0.06 mL, about 0.07 mL, about 0.08 mL, about 0.09 mL, about 0.10 mL, about 0.11 mL, about 0.12 mL, about 0.13 mL, about 0.14 mL, or about 0.15 mL per cochlea, depending on the population.

In some embodiments, a dosing regimen comprises delivery in a concentration of about 1.0e13 VG/kg, about 1.1 e13 VG/kg, about 1.2e13 VG/kg, about 1.3e13 VG/kg, about 1.4e13 VG/kg, about 1.5e13 VG/kg, about 1.6e13 VG/kg, about 1.7e13 VG/kg, about 1.8e13 VG/kg, about 1.9e13 VG/kg, about 2.0e13 VG/kg, about 2.1e13 VG/kg, about 2.2e13 VG/kg, about 2.3e13 VG/kg, about 2.4e13 VG/kg, about 2.5e13 VG/kg, about 2.6e13 VG/kg, about 2.7e13 VG/kg, about 2.8e13 VG/kg, about 2.9e13 VG/kg, about 3.0e13 VG/kg, about 3.1e13 VG/kg, about 3.2e13 VG/kg, about 3.3e13 VG/kg, about 3.4e13 VG/kg, about 3.5e13 VG/kg, about 3.6e13 VG/kg, about 3.7e13 VG/kg, about 3.8e13 VG/kg, about 3.9e13 VG/kg, about 4.0e13 VG/kg, about 4.1e13 VG/kg, about 4.2e13 VG/kg, about 4.3e13 VG/kg, about 4.4e13 VG/kg, about 4.5e13 VG/kg, about 4.6e13 VG/kg, about 4.7e13 VG/kg, about 4.8e13 VG/kg, about 4.9e13 VG/kg, about 5.0e13 VG/kg, about 1.0e14 VG/kg, about 1.le14 VG/kg, about 1.2e14 VG/kg, about 1.3e14 VG/kg, about 1.4e14 VG/kg, about 1.5e14 VG/kg, about 1.6e14 VG/kg, about 1.7e14 VG/kg, about 1.8e14 VG/kg, about 1.9e14 VG/kg, about 2.0e14 VG/kg.

In some embodiments, a method disclosed herein evaluates safety and tolerability of escalating doses of a preparation, a construct(s), a virion, a population of virions, a composition, or a pharmaceutical composition disclosed herein administered via systemic administration to a subject, e.g., 1 to 80 years of age, with a disease described herein.

In some embodiments, any of the methods disclosed herein comprise an evaluation of safety and tolerability of a preparation, a construct(s), a virion, a population of virions, a composition, or a pharmaceutical composition disclosed herein. In some embodiments, evaluation of the efficacy of a preparation, a construct(s), a virion, a population of virions, a composition, or a pharmaceutical composition disclosed herein to treat a disease described herein, is performed in a randomized, controlled setting (using a concurrent, non-intervention observation arm).

### 5. Exemplary Diseases

In some embodiments, compositions, preparations, constructs, virions, population of virions, host cells, and/or pharmaceutical compositions described herein may be used for prevention and/or treatment of various diseases.

In some embodiments, a disease is selected from endothelial dysfunction, cystic fibrosis, cardiovascular disease, kidney disease, renal disease, ocular disease, cancer, hemoglobinopathy, anemia, hemophilia, myeloproliferative disorder, coagulopathy, sickle cell disease, alpha-thalassemia, beta-thalassemia, hemophilia (e.g., hemophilia A), Fanconi anemia, familial intrahepatic cholestasis, epidermolysis bullosa, Fabry, Gaucher, Nieman-Pick A, Nieman-Pick B, GM1 Gangliosidosis, Mucopolysaccharidosis (MPS) I (Hurler, Scheie, Hurler/Scheie), MPS II (Hunter), MPS VI (Maroteaux-Lamy), hematologic cancer, hemochromatosis, hereditary hemochromatosis, juvenile hemochromatosis, cirrhosis, hepatocellular carcinoma, pancreatitis, diabetes mellitus, cardiomyopathy, arthritis, hypogonadism, cardiac (or heart) disease, heart attack, hypothyroidism, glucose intolerance, arthropathy, liver fibrosis, Wilson's disease, ulcerative colitis, Crohn's disease, Tay-Sachs disease, neurodegenerative disorder, Spinal muscular atrophy type 1, Huntington's disease, Canavan's disease, lysosomal storage diseases, rheumatoid arthritis, inflammatory bowel disease, psoriatic arthritis, juvenile chronic arthritis, psoriasis, and ankylosing spondylitis, and autoimmune disease, neurodegenerative disease (e.g., Alzheimer's disease, Parkinson's disease, Huntington's disease, ataxias), inflammatory disease, inflammatory bowel disease, Crohn's disease, rheumatoid arthritis, lupus, multiple sclerosis, chronic obstructive pulmony disease/COPD, pulmonary fibrosis, Sjogren's disease, hyperglycemic disorders, type I diabetes, type II diabetes, insulin resistance, hyperinsulinemia, insulin-resistant diabetes (e.g. Mendenhall's Syndrome, Werner Syndrome, leprechaunism, and lipoatrophic diabetes), dyslipidemia, hyperlipidemia, elevated low-density lipoprotein (LDL), depressed highdensity lipoprotein (HDL), elevated triglycerides, metabolic syndrome, liver disease, renal disease, cardiovascular disease, ischemia, stroke, complications during reperfusion, muscle degeneration, atrophy, symptoms of aging (e.g., muscle atrophy, frailty, metabolic disorders, low grade inflammation, atherosclerosis, stroke, age-associated dementia and sporadic form of Alzheimer's disease, pre-cancerous states, and psychiatric conditions including depression), spinal cord injury, arteriosclerosis, infectious diseases (e.g., bacterial, fungal, viral), AIDS, tuberculosis, defects in embryogenesis, infertility, lysosomal storage diseases, activator deficiency/GM2 gangliosidosis, alpha-mannosidosis, aspartylglucoaminuria, cholesteryl ester storage disease, chronic hexosaminidase A deficiency, cystinosis, Danon disease, Farber disease, fucosidosis, galactosialidosis, Gaucher Disease (Types I, II and III), GM1 Gangliosidosis, (infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Neuronal ceroid lipofuscinoses, CLN6 disease, Jansky-Bielschowsky disease, Pompe disease, pycnodysostosis, Sandhoff disease, Schindler disease, and Wolman disease.

In some embodiments, a disease is a kidney disease. In some embodiments, a disease is Alport syndrome. In some embodiments, a disease is Fabry disease. In some embodiments, a disease is autosomal dominant polycystic kidney disease (PKD). In some embodiments, a disease is congenital nephrotic syndrome.

In some embodiments, a disease is a cardiac (or heart) disease. In some embodiments, a cardiac (or heart) disease is hypertrophic cardiomyopathy. In some embodiments, a disease is dilated cardiomyopathy.

In some embodiments, compositions, preparations, constructs, virions, population of virions, host cells, and/or pharmaceutical compositions comprising a protoparvovirus variant VP1 capsid polypeptide are useful for transducing a hematopoietic cells, hematopoietic progenitor cell, hematopoietic stem cells, erythroid lineage cell, megakaryocyte, erythroid progenitor cell (EPC), CD34+ cell, CD36+ cell, mesenchymal stem cell, nerve cell, intestinal cells, intestinal stem cell, gut epithelial cell, endothelial cells, lung cells, enterocyte, liver cell (e.g., hepatocyte, hepatic stellate cells (HSCs), Kupffer cells (KCs), liver sinusoidal endothelial cells (LSECs)), brain microvascular endothelial cell (BMVECs), erythroid progenitor cell, lymphoid progenitor cells, B lymphoblast cell, T cells, B cells, basophilic Endemic Burkitt Lymphoma (EBL), polychromatic erythroblast, orthochromatic erythroblast, kidney cells, or cardiac (or heart) cells. In some embodiments, compositions, preparations, constructs, virions, population of virions, host cells, and/or pharmaceutical compositions comprising a protoparvovirus variant VP1 capsid polypeptide are useful for transducing a testes cell, an oocyte, a medulla cell, a striatum cell, a spinal cord (or chord) cell, or a duodenum cell. In some embodiments, compositions, preparations, constructs, virions, population of virions, host cells, and/or pharmaceutical compositions comprising a protoparvovirus variant VP1 capsid polypeptide are useful for transducing kidney cells. In some embodiments, compositions, preparations, constructs, virions, population of virions, host cells, and/or pharmaceutical compositions comprising a protoparvovirus variant VP1 capsid polypeptide are useful for transducing cardiac (or heart) cells. In some embodiments, compositions, preparations, constructs, virions, population of virions, host cells, and/or pharmaceutical compositions comprising a protoparvovirus variant VP1 capsid polypeptide are useful for transducing brain cells.

In addition, in some embodiments, compositions, preparations, constructs, virions, population of virions, host cells, and/or pharmaceutical compositions described herein are particularly useful in delivering a nucleic acid (e.g., a therapeutic nucleic acid, e.g., a transgene) in vivo (e.g., administering directly to a subject, e.g., targeting a specific tissue via viral tropism), as well as in vitro or ex vivo (obtaining a plurality of cells from a subject, transducing said cells using virions, and administering the subject an effective number of transduced cells).

In some embodiments, an exemplary disease is hemochromatosis as described by “Protoparvovirus and tetraparvovirus compositions and methods for gene therapy” published as WO2022140683A1 on Jun. 30, 2022, the entire contents of which are hereby incorporated by reference herein.

In some embodiments, an exemplary disease includes inflammatory bowel disease (IBD) as described by “Protoparvovirus and tetraparvovirus compositions and methods for gene therapy” published as WO2022140683A1 on Jun. 30, 2022, the entire contents of which are hereby incorporated by reference herein.

In some embodiments, an exemplary disease includes autophagy-related diseases as described by “Protoparvovirus and tetraparvovirus compositions and methods for gene therapy” published as WO2022140683A1 on Jun. 30, 2022 the entire contents of which are hereby incorporated by reference herein.

In some embodiments, an exemplary disease includes inflammatory disorders as described by “Protoparvovirus and tetraparvovirus compositions and methods for gene therapy” published as WO2022140683A1 on Jun. 30, 2022, the entire contents of which are hereby incorporated by reference herein.

In some embodiments, an exemplary disease includes cancer as described by “Protoparvovirus and tetraparvovirus compositions and methods for gene therapy” published as WO2022140683A1 on Jun. 30, 2022 the entire contents of which are hereby incorporated by reference herein.

In some embodiments, an exemplary disease includes familial intrahepatic cholestasis as described by “Protoparvovirus and tetraparvovirus compositions and methods for gene therapy” published as WO2022140683A1 on Jun. 30, 2022 the entire contents of which are hereby incorporated by reference herein.

In some embodiments, an exemplary disease includes Wilson disease as described by “Protoparvovirus and tetraparvovirus compositions and methods for gene therapy” published as WO2022140683A1 on Jun. 30, 2022, the entire contents of which are hereby incorporated by reference herein.

In some embodiments, an exemplary disease includes lysosomal Storage Disorders as described by “Protoparvovirus and tetraparvovirus compositions and methods for gene therapy” published as WO2022140683A1 on Jun. 30, 2022, the entire contents of which are hereby incorporated by reference herein.

In some embodiments, an exemplary disease includes epidermolysis bullosa as described by “Protoparvovirus and tetraparvovirus compositions and methods for gene therapy” published as WO2022140683A1 on Jun. 30, 2022, the entire contents of which are hereby incorporated by reference herein.

In some embodiments, an exemplary disease includes hematologic diseases as described by “Protoparvovirus and tetraparvovirus compositions and methods for gene therapy” published as WO2022140683A1 on Jun. 30, 2022, the entire contents of which are hereby incorporated by reference herein.

In some embodiments, an exemplary disease includes type I diabetes as described by “Protoparvovirus and tetraparvovirus compositions and methods for gene therapy” published as WO2022140683A1 on Jun. 30, 2022, the entire contents of which are hereby incorporated by reference herein.

In some embodiments, an exemplary disease includes hemophilia A as described by “Protoparvovirus and tetraparvovirus compositions and methods for gene therapy” published as WO2022140683A1 on Jun. 30, 2022, the entire contents of which are hereby incorporated by reference herein.

In some embodiments, an exemplary disease includes neurodegenerative disorders and neuromuscular disorders including but not limited to spinal muscular atrophy type 1, Huntington's disease, Canavan's disease, and lysosomal storage diseases as described herein.

In some embodiments, an exemplary disease includes ocular disorders.

The disclosure is further described in detail by reference to the following experimental examples. These examples are provided for purposes of illustration only, and are not intended to be limiting unless otherwise specified. Thus, the disclosure should in no way be construed as being limited to the following examples, but rather should be construed to encompass any and all variations that become evident as a result of the teaching provided herein.

For example, other assays, including those described in the Example section herein as well as those that are known in the art, can also be used in accordance with the present disclosure.

### EXAMPLES

### Example 1: Alignments of Protoparvovirus VP1 Capsid Amino Acid Sequences Across Exemplary Protoparvovirus Species Showed Significant Conservation of a Splice Variant that Eliminates a Stretch of Amino Acid Residues within a Protoparvovirus VP1 Capsid Polypeptide

The present example identifies significantly conserved characteristic sequence elements within a protoparvovirus VP1 capsid polypeptide (e.g., within a VP1 unique region (VP1u)).

Expression of protoparvovirus capsid polypeptides in host cell systems, including baculovirus-Sf9 systems, is challenging due to cell toxicity. Without wishing to be bound to any theory, cell toxicity may be due to protoparvovirus VP1 capsid polypeptide retention in cell cytoplasm, which can result in protein aggregation and subsequent toxicity as described herein.

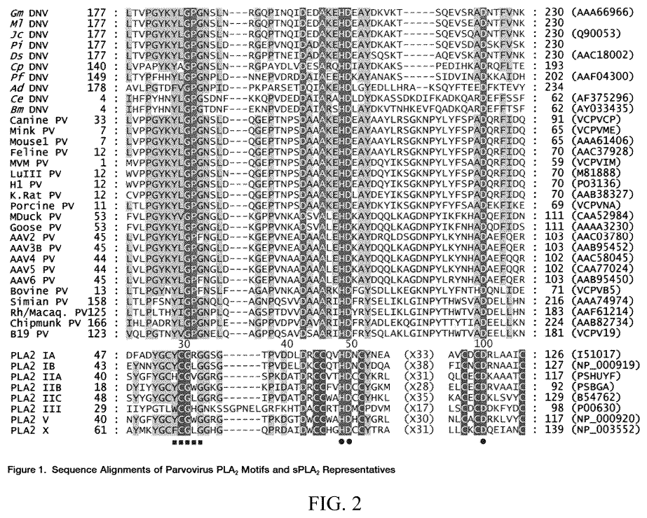

FIG. 1 shows alignments of an N-terminus region of exemplary protoparvovirus VP1u within a VP1 capsid polypeptide. Alignments depicted by FIG. 1 reveal significant conservation of a stretch of amino acid residues (“aa_del” motif) within exemplary protoparvovirus species including bufavirus (BuV), cutavirus (CuV), tusavirus (TuV), minute virus of mice (MVM), canine parvovirus (CPV), and feline panleukopenia virus (FPV). Alignments depicted by FIG. 1 also show significant conservation of a putative nuclear localization signal sequence (NLS) upstream of a five amino acid motif. Alignments depicted by FIG. 1 also show highly conserved PLA2 motif residues downstream of an aa_del motif (see FIG. 2).

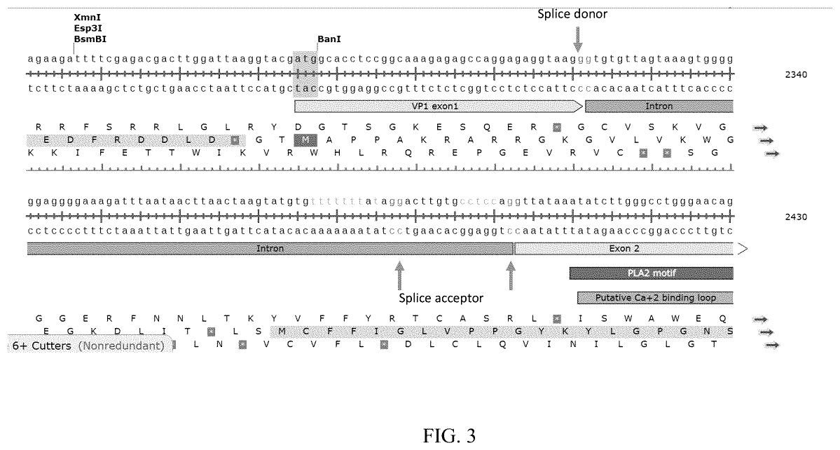

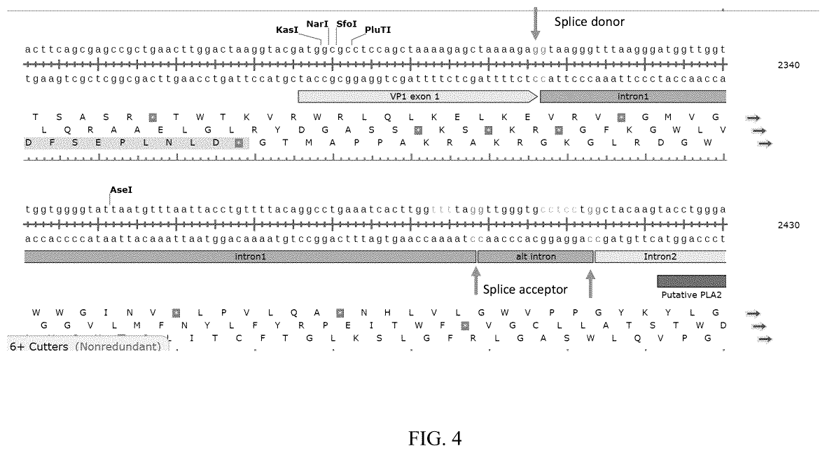

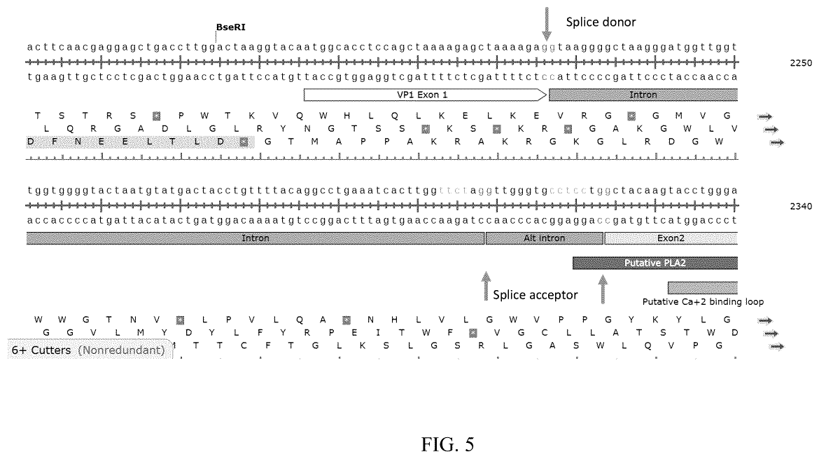

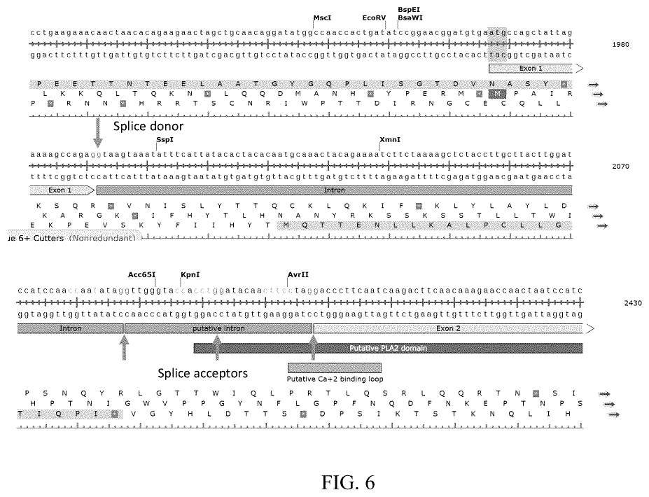

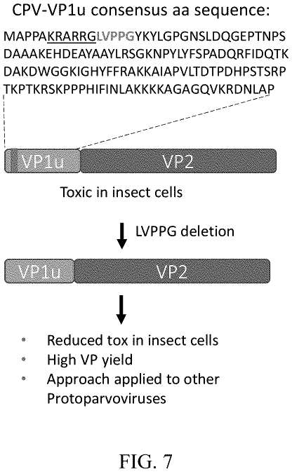

The present disclosure recognizes that an adjacent splice donor sequence and a splice acceptor sequence occurs downstream of a conserved NLS and upstream of a conserved PLA2 within this region that results in deletion of a conserved amino acid motif (see FIG. 7). The present disclosure also recognizes that this splice variant can reduce VP1 capsid polypeptide toxicity. The present disclosure also recognizes that this splice variant can increase virion potency. It is an insight of the present disclosure that a splice variant does not necessarily occur in host cells as described herein. Moreover, as described herein, it is an insight of the present disclosure that adjacent splice donor/acceptor sequences between a NLS and initiation of a PLA2 motif are conserved across a variety of protoparvovirus species. For example, canine parvovirus (CPV) sequence analysis depicted in FIG. 3 shows adjacent splice donor/acceptor sequences between a NLS (KRARRG) and initiation of a PLA2 motif that results in deletion of a five amino acid motif. As another example, FIG. 4 shows two adjacent donor/acceptor sequences between a NLS (KRAKRG) and a PLA2 motif that can result in deletion of a five amino acid motif in a reference minute virus of mice (MVM) VP1 capsid polypeptide sequence. Moreover, FIG. 5 shows adjacent splice acceptor/donor sequences between a NLS (KRAKRG) and a PLA2 motif that can result in deletion of a five amino acid motif in a reference rat H-1 parvovirus (H-1PV) VP1 capsid polypeptide sequence. As another example, FIG. 6 shows adjacent donor/acceptor sequences between a NLS (KARG) and a PLA2 motif that can result in deletion or partial deletion of a five amino acid motif in a reference cutavirus (CuV) VP1 capsid polypeptide sequence.

Accordingly, without wishing to be bound to any theory, it is an insight of the present example that protoparvovirus VP1 capsid polypeptide toxicity can be reduced by engineering compositions, preparations, constructs, virions, population of virions, and host cells comprising a protoparvovirus variant VP1 capsid polypeptide as described herein.

### Example 2: A Protoparvovirus Variant VP1 Capsid Polypeptide in Host Cells Exhibited Increased Potency and Reduced Toxicity in Host Cells

The present example provides exemplary compositions, preparations, constructs, virions, population of virions, and host cells for gene therapy and related methods that show increased potency and reduced toxicity in host cells as described herein.

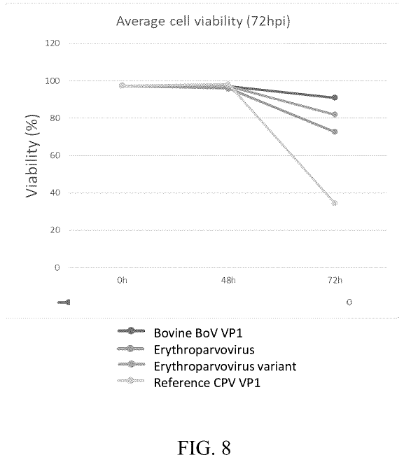

Virions comprising a CPV reference VP1 capsid polypeptide encoded by a CPV reference VP1 capsid coding sequence according to SEQ ID NO: 126) were generated and tested in host cells according to standard protocols. As shown in FIG. 8, a CPV reference VP1 capsid polypeptide showed elevated toxicity in insect cells at 72 hours post-infection (hpi), affecting VP1 capsid polypeptide yield, compared to other genuses in family parvovirinae (such as bocavirus or erythroparvovirus).

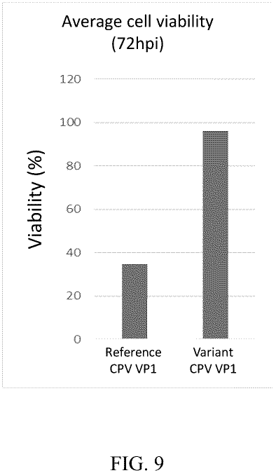

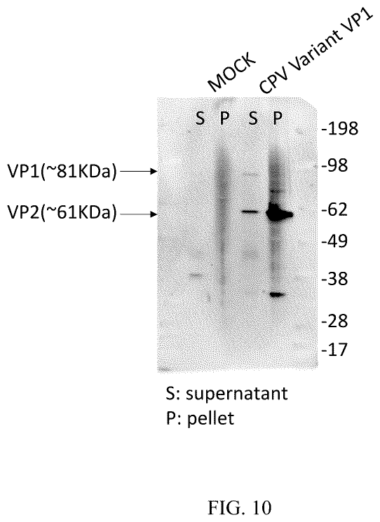

An exemplary construct comprising deletion of a five amino acid motif (LVPPG-SEQ ID NO: 1) immediately downstream of a NLS within the CPV VP1 capsid polypeptide (e.g., a construct according to SEQ ID NO: 121) was designed and tested in host cells according to standard protocols. As described by Example 1, this deleted region is conserved across other protoparvovirus species. As shown in FIG. 9, a CPV variant VP1 capsid polypeptide construct showed more than double the average percent cell viability at 72 hpi compared to a CPV reference VP1 capsid polypeptide. Further, FIG. 10 depicts detection of CPV VP1 and VP2 capsid polypeptides by Western Blot in the supernatant and pellet of insect (Sf9) cells infected with a baculovirus construct (BEV) expressing a CPV variant VP1 construct. The present disclosure recognizes that other exemplary protoparvovirus variant VP1 capsid polypeptides described herein can be used.

Accordingly, in some embodiments, the present example demonstrates a protoparvovirus variant VP1 capsid polypeptides described herein increases potency and reduces toxicity of exemplary virions comprising a protoparvovirus variant VP1 capsid polypeptide in host cells.

### Example 3: Exemplary Constructs Comprising a Protoparvovirus Variant VP1 Capsid Polypeptide in a Host Cell Increased VP1 Initiation Relative to a Protoparvovirus Reference VP1 Capsid Polypeptide

The present example demonstrates that modifications and/or selections of components of constructs encoding a protoparvovirus VP1 capsid polypeptide or protoparvovirus variant VP1 capsid polypeptide described herein, can increase VP1 initiation in host cells. Moreover, the present example demonstrates that modifications and/or selections of components of constructs encoding a protoparvovirus VP1 capsid polypeptide or protoparvovirus variant VP1 capsid polypeptide described herein, can increase potency in host cells.

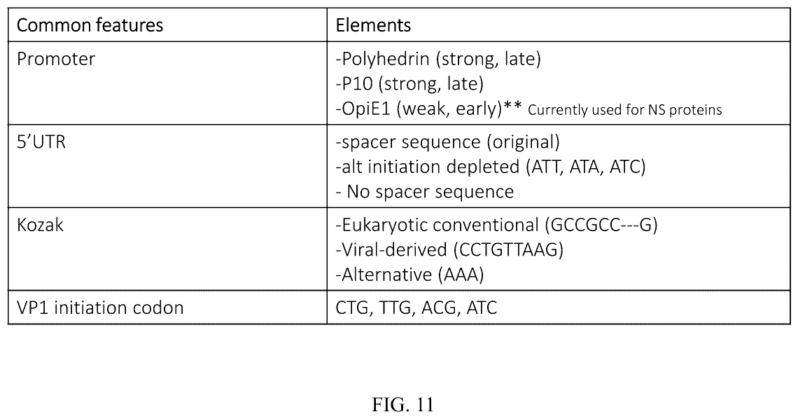

FIG. 11 depicts exemplary protoparvovirus construct elements that can improve production and/or reduce toxicity of protoparvovirus variant VP1 capsid polypeptides in host cells, according to an embodiment of the present disclosure. In some embodiments, a construct element includes one or more of an expression control sequence, a 5′ UTR, a VP1 translation initiation sequence, or a combination thereof.

In some embodiments, selection of an expression control sequence having certain components can improve production of a protoparvovirus VP1 capsid polypeptide. In some embodiments, selection of an expression control sequence having certain characteristics can reduce toxicity of a protoparvovirus VP1 capsid polypeptide. In some embodiments a characteristic is strong expression. In some embodiments a characteristic is weak expression. In some embodiments a characteristic is delayed expression. In some embodiments a characteristic is early expression.

For example, polyhedrin is an exemplary expression control sequence that can initiate strong and/or late expression of a VP1 capsid polypeptide. As another example, P10 is an exemplary expression control sequence that can initiate strong and/or late expression of a VP1 capsid polypeptide. Moreover, OpiE1 is an exemplary expression control sequence that can initiate weak and/or early expression of a VP1 capsid polypeptide.

Among other things, the present example recognizes that selection of a 5′ UTR sequence can improve production and/or reduce toxicity of a VP1 capsid polypeptide. In some embodiments, a 5′UTR sequence is a stretch of nucleotides between an expression control sequence and a VP1 capsid coding sequence (referred to herein as “a nucleotide spacer sequence”).

In some embodiments, a 5′UTR sequence comprises a nucleotide spacer sequence. In some embodiments, a 5′ UTR sequence comprises a nucleotide spacer sequence that does not comprise an alternative translation initiation sequence (e.g., so translation does not start before a VP1 capsid coding sequence). In some embodiments, a 5′ UTR sequence comprises a nucleotide spacer sequence and a Kozak consensus sequence. In some embodiments, a 5′ UTR sequence does not comprise a nucleotide spacer sequence. In some embodiments, there is no nucleotide spacer sequence between an expression control sequence and a VP1 capsid coding sequence.

In some embodiments, a 5′ UTR sequence comprises a nucleotide spacer sequence as shown in Table 6.

TABLE 6

| Sequence | | SEQ ID |
|---|---|---|
| Name | Sequence | NO: |
| Exemplary | ATTCCGGATTATTCATACCGT | SEQ ID |
| Nucleotide | CCCACCATCGGGCGCGGATCT | NO: 123 |
| Spacer | | |
| Sequence 1 | | |
| Exemplary | ACTCCGGACTACTGATACCGT | SEQ ID |
| Nucleotide | CCCACTTTCGGGCGCTTACCT | NO: 124 |
| Spacer | | |
| Sequence 2 | | |
| (without | | |
| alternative | | |
| translation | | |
| initiation | | |
| sequences) | | |

In some embodiments, a Kozak sequence comprises a eukaryotic (GCCGCC - - - G), viral-derived (CCTGTTAAG), or alternate sequence (AAA).

In some embodiments, a protoparvovirus variant VP1 capsid polypeptide construct comprises an alternative translation initiation sequence such as CTG, ATC, TTG and ACG.

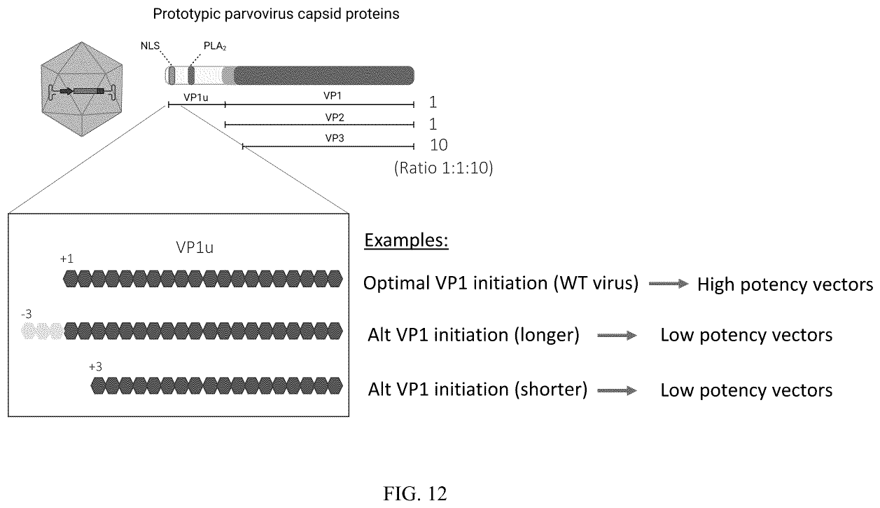

Moreover, the present example describes that leaky scanning of an mRNA sequence for expression of VP1, VP2, and VP3 capsid polypeptides in a suitable ratio, for example, a VP1: VP2: VP3 ratio of 1:1:10, can result in alternate initiation of a VP1 capsid polypeptide. As described by this Example, alternative initiation of a VP1 capsid polypeptide leads to a longer or shorter VP1 capsid polypeptide which can negatively impact virion potency, as shown in FIG. 12. Appropriate VP1 capsid polypeptide initiation leads to high potency virions.

Accordingly, in some embodiments, the present disclosure describes compositions, preparations, constructs, virions, population of virions, and host cells comprising a protoparvovirus variant VP1 capsid polypeptide can exhibit increased VP1 initiation relative to a reference VP1 capsid polypeptide.

### Example 4: Increased AAV Genome Trans-Encapsidation Generates a High Filled/Empty Capsid Ratio in a Host System

The present example demonstrates that modifications and/or selections of components of constructs encoding a protoparvovirus VP1 capsid polypeptide or protoparvovirus variant VP1 capsid polypeptide described herein, can increase AAV genome trans-encapsidation within a protoparvovirus variant VP1 capsid polypeptide in host cells.

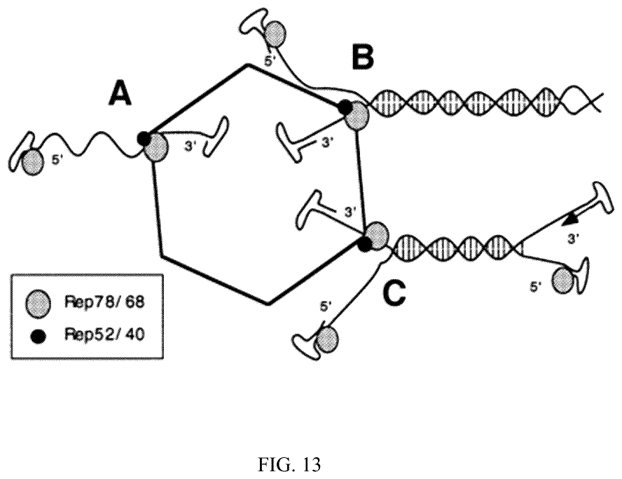

Parvovirus non-structural proteins play a key role in different steps of a virus life cycle, from DNA replication and transcription regulation to genome packaging. While an N-terminus region of a full-length NS (e.g., Rep78 in AAVs or NS1 in autonomous parvoviruses) participates in genome replication, recognizing the viral genome in a sequence-specific manner, a C-terminus region (e.g., Rep 52/40 in AAVs) contains an SF3 helicase domain. A SF3 helicase domain acts as a motor to incorporate a viral genome into a preformed capsid, as shown in FIG. 13 (see also King et al. EMBO J. 2001 Jun. 15; 20(12): 3282-91, doi: 10.1093/emboj/20.12.3282, the entire contents of which are hereby incorporated by reference herein). In some embodiments, without wishing to be bound to any theory, the present disclosure describes that an SF3 helicase domain of a NS1 of a protoparvovirus can apply a force to incorporate an AAV genome into a protoparvovirus variant VP1 capsid polypeptide in an ATP-dependent manner. Without wishing to be bound to any theory, this function is believed to take place via an AAV packaging complex comprising an immobilized helicase complex, composed of large and small Rep proteins, on a capsid surface. As shown in FIG. 13, a genome is translocated through an AAV packaging complex and into a capsid either (A) as a single-stranded molecule using the initial ‘scanning’ function before the first duplexed base pairs are encountered or (B) by unwinding a double-stranded dimer or multimer genome on a capsid surface at the same time or (C) simultaneous replication (arrow) of a double-stranded monomer genome being packaged.

In view of functional co-evolution of parvovirus NS proteins and respective capsids, it is an insight of the present disclosure that co-expression of the C-terminus region of a NS protein from a cognate autonomous protoparvovirus provides a more efficient NS-capsid interaction, thus improving packaging of an AAV-derived genome (e.g., a transgene) into a respective capsid via a helicase domain, in a sequence-independent manner.

Accordingly, in some embodiments, the present disclosure describes compositions, preparations, constructs, virions, population of virions, and host cells can exhibit increased encapsidation via co-expression of NS1.

### Example 5: Exemplary Virions Comprising a Parvovirus VP1 Capsid Polypeptide Produced in Host HEK293 Cells

The present Example confirms that exemplary compositions, preparations, nucleotide sequences, and methods described herein can be used to produce virions comprising a protoparvovirus VP1 capsid polypeptide in mammalian host cells e.g., HEK293 cells.

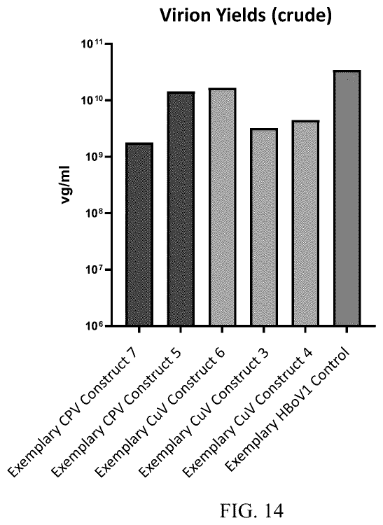

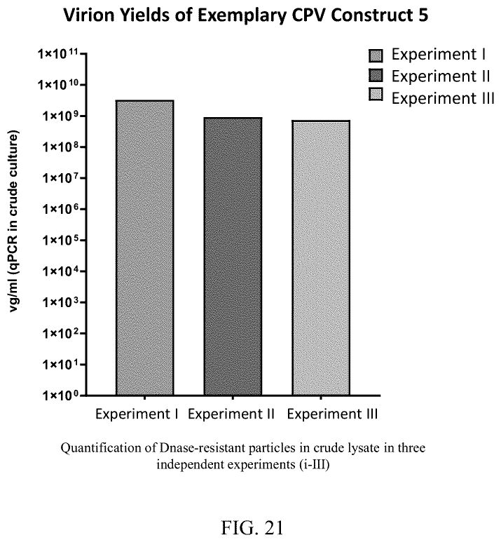

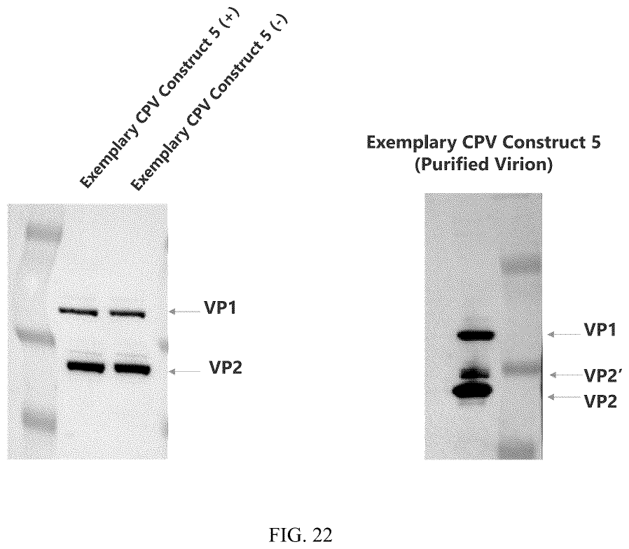

As shown in FIG. 14, (1) virions comprising a CPV VP1 capsid polypeptide encoded by a VP1 capsid coding sequence according to SEQ ID NO: 130 (Exemplary CPV Construct 5) and (2) virions comprising a CuV VP1 capsid polypeptide encoded by a VP1 capsid coding sequence according to SEQ ID NO: 139 (Exemplary CuV Construct 6) produced similar virion yields (vg/mL) in host HEK293 cells relative to virions comprising an exemplary control HBoV1 capsid polypeptide. Moreover, as shown in FIG. 14, (3) virions comprising a CPV VP1 capsid polypeptide encoded by a VP1 capsid coding sequence according to SEQ ID NO: 148 (Exemplary CPV Construct 7), (4) virions comprising a CuV VP1 capsid polypeptide encoded by a VP1 capsid coding sequence according to SEQ ID NO: 133 (Exemplary CuV Construct 3), and (5) virions comprising a CuV VP1 capsid polypeptide encoded by a VP1 capsid coding sequence according to SEQ ID NO: 134 (Exemplary CuV Construct 4) generated reasonable quantities of virion yields (vg/mL) in host HEK293 cells, despite being an order magnitude less than quantities of virion yields (vg/mL) of (6) virions comprising an exemplary control HBoV1 capsid polypeptide in host HEK293 cells. FIG. 21 shows comparable virion yields (vg/mL) of virions comprising a CPV VP1 capsid polypeptide encoded by a VP1 capsid coding sequence according to SEQ ID NO: 130 (Exemplary CPV Construct 5) produced in host HEK293T cells across three independent experiments.

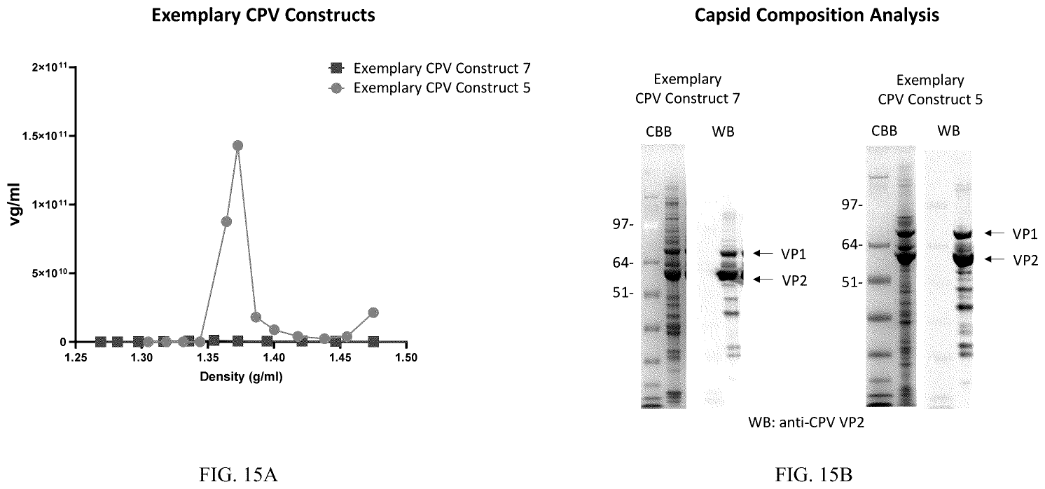

FIG. 15A shows fractions comprising filled virions (or particles) that were detected and isolated via ultracentrifugation in CsCl of virions comprising a CPV VP1 capsid polypeptide encoded by a VP1 capsid coding sequence according to SEQ ID NO: 148 (Exemplary CPV Construct 7) and virions comprising a CPV VP1 capsid polypeptide encoded by a VP1 capsid coding sequence according to SEQ ID NO: 130 (Exemplary CPV Construct 5). FIG. 15B shows a western blot analysis of capsid composition and amounts of VP1 and VP2 capsid polypeptides of virions comprising a CPV VP1 capsid polypeptide encoded by a VP1 capsid coding sequence according to SEQ ID NO: 148 (Exemplary CPV Construct 7), and virions comprising a CPV VP1 capsid polypeptide encoded by a VP1 capsid coding sequence according to SEQ ID NO: 130 (Exemplary CPV Construct 5) produced in host HEK293 cells.

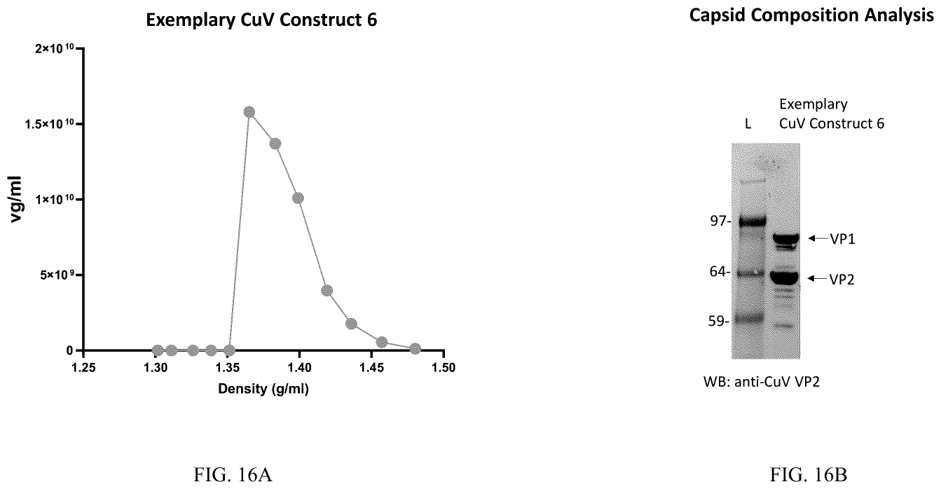

FIG. 16A shows fractions comprising filled virions (or particles) that were detected and isolated via ultracentrifugation in CsCl of virions comprising a CuV VP1 capsid polypeptide encoded by a VP1 capsid coding sequence according to SEQ ID NO: 139 (Exemplary CuV Construct 6) produced in HEK293 cells. FIG. 16B shows a western blot analysis of capsid composition and amounts of VP1 and VP2 capsid polypeptides of virions comprising CuV VP1 capsid polypeptide encoded by a VP1 capsid coding sequence according to SEQ ID NO: 139 (Exemplary CuV Construct 6) produced in host HEK293 cells.

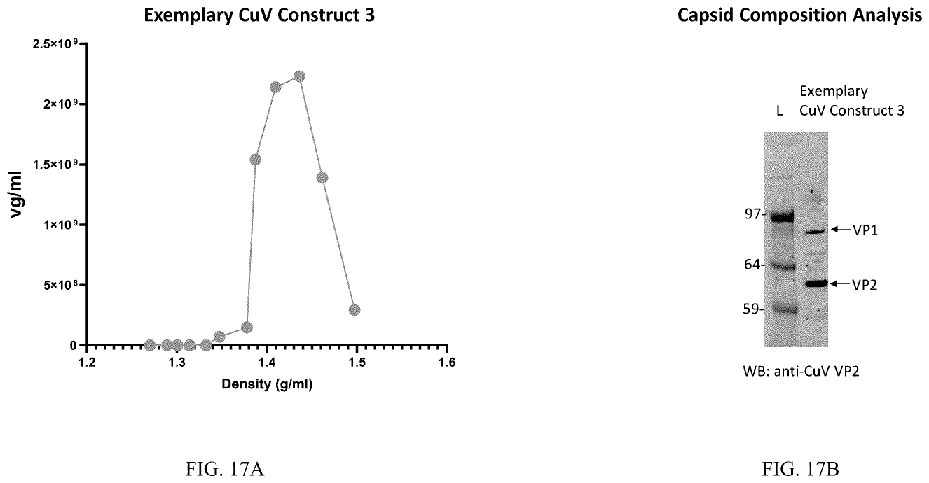

FIG. 17A shows fractions comprising filled virions (or particles) that were detected and isolated via ultracentrifugation in CsCl of virions a CuV VP1 capsid polypeptide encoded by a VP1 capsid coding sequence according to SEQ ID NO: 133 (Exemplary CuV Construct 3) produced in HEK293 cells. FIG. 17B shows a western blot analysis of capsid composition and amounts of VP1 and VP2 capsid polypeptides of virions comprising a CuV VP1 capsid polypeptide encoded by a VP1 capsid coding sequence according to SEQ ID NO: 133 (Exemplary CuV Construct 3) produced in host HEK293 cells.

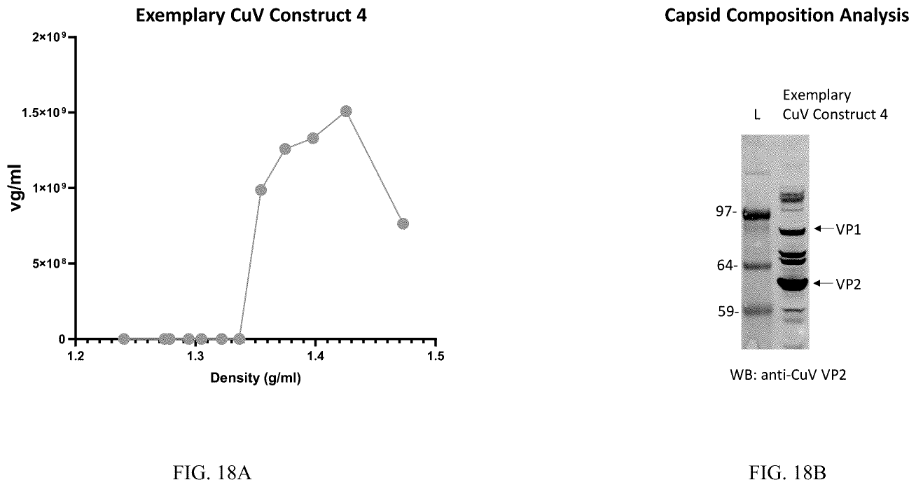

FIG. 18A shows fractions comprising filled virions (or particles) that were detected and isolated via ultracentrifugation in CsCl of virions comprising a CuV VP1 capsid polypeptide encoded by a VP1 capsid coding sequence according to SEQ ID NO: 134 (Exemplary CuV Construct 4) produced in HEK293 cells. FIG. 18B shows a western blot analysis of capsid composition and amounts of VP1 and VP2 capsid polypeptides of virions comprising a CuV VP1 capsid polypeptide encoded by a VP1 capsid coding sequence according to SEQ ID NO: 134 (Exemplary CuV Construct 4) produced in host HEK293 cells.

Therefore, the data shown in FIGS. 14-18B confirm efficient and robust production of virions comprising a CPV or CuV VP1 capsid polypeptide in mammalian cells. Moreover, the data also confirm that construct component design can influence virion production in host cells.

The present Example can be used with other protoparvovirus capsid polypeptides beyond CPV and CuV capsid polypeptides as described herein.

Accordingly, the present Example confirms that exemplary compositions, preparations, nucleotide sequences, and methods described herein can be used to produce virions comprising a protoparvovirus VP1 capsid polypeptide in mammalian host cells. Moreover, the present Example confirms that virions comprising an exemplary CPV VP1 capsid polypeptide as described herein can be produced in mammalian cells. Moreover, the present Example confirms that virions comprising an exemplary CuV VP1 capsid polypeptide as described herein can be produced in mammalian cells.

### Example 6: Exemplary Virions Comprising a CPV Capsid Interact with a Transferrin Receptor

The present Example provides exemplary compositions, preparations, constructs, virions, population of virions, which can interact with a transferrin receptor (TfR). In particular, for instance, the present Example demonstrates that a protoparvovirus VP1 capsid coding sequence encoding a VP1 capsid polypeptide sequence as described herein produced virions that were efficiently transduced into human neuroblastoma (e.g., SH-SY-5Y) cells and human kidney (e.g., HEK293T) cells.

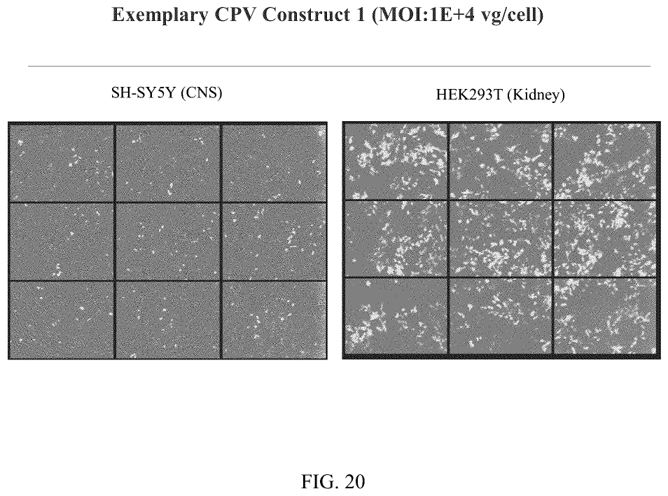

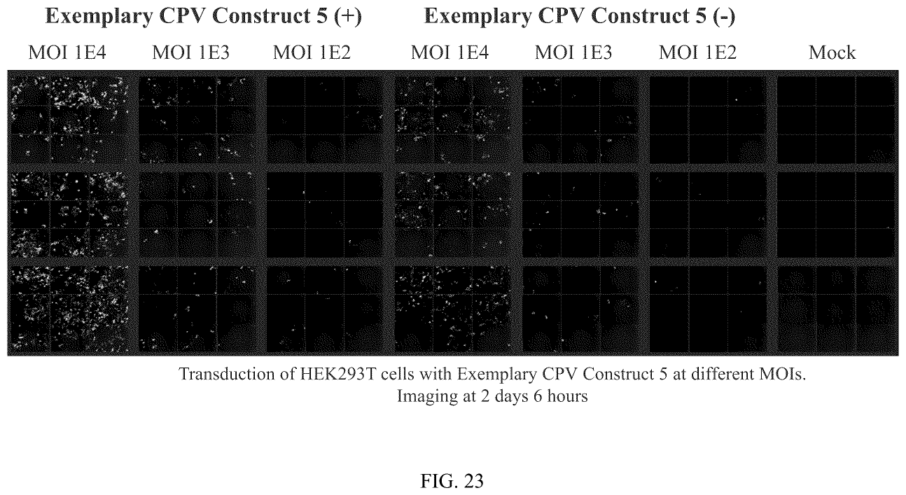

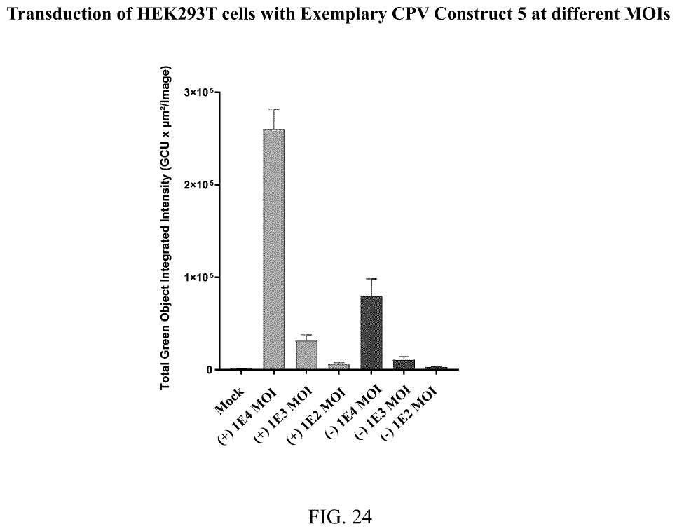

Virions comprising a CPV VP1 capsid polypeptide encoded by a VP1 capsid coding sequence according to SEQ ID NO: 126 (Exemplary CPV Construct 1) produced in HEK293T cells via triple transfection showed transduction of human neuroblastoma cell line SH-SY5Y cells and kidney cell line HEK293T cells, as shown in FIG. 20. Initial manufacturability shows titers of about 1E9 vg/ml in crude (data not shown). FIG. 23 shows fluorescence imaging of kidney cell line HEK293T cells transduced with MOI 1E4 vg/cell, 1E3 vg/cell, and 1E2 vg/cell of virions comprising a CPV VP1 capsid polypeptide encoded by a VP1 capsid coding sequence according to SEQ ID NO: 130 (Exemplary CPV Construct 5) with (+) and without (−) trypsin conditions. FIG. 24 shows a bar graph depicting GFP transgene expression as measured by GCU×μm2 per image of HEK293T cells transduced with MOI 1E4 vg/cell, 1E3 vg/cell, and 1E2 vg/cell of virions comprising a CPV VP1 capsid polypeptide encoded by a VP1 capsid coding sequence according to SEQ ID NO: 130 (Exemplary CPV Construct 5) with (+) and without (−) trypsin conditions. As shown in FIGS. 23-24, virions comprising a CPV VP1 capsid polypeptide encoded by a VP1 capsid coding sequence according to SEQ ID NO: 130 (Exemplary CPV Construct 5) showed robust transduction of HEK293T cells at MOI 1E4 vg/cell.

Accordingly, the present Example confirms that exemplary compositions, preparations, constructs, virions, and population of cells comprising recombinant virions can be produced in mammalian host cells. Moreover, the present Example confirms transduction of the described virions in human cells. The present Example also confirms that virions comprising an exemplary CPV VP1 capsid polypeptide as described herein can transduce mammalian cells as described herein.

The present Example can be used with other protoparvovirus capsid polypeptides beyond CPV capsid polypeptides as described herein.

### EXEMPLARY EMBODIMENTS

Embodiment 1. A construct comprising a VP1 capsid coding sequence operably linked to an expression control sequence, wherein the VP1 capsid coding sequence encodes a protoparvovirus variant VP1 capsid polypeptide having an amino acid sequence that:
(i) shows at least 70% overall sequence identity with that of a protoparvovirus reference VP1 capsid polypeptide selected from the group consisting of those in Table 3B, which reference polypeptide includes an amino acid sequence element as set forth in SEQ ID NOs: 1-3 or both; and
(ii) includes at least one sequence variation (e.g., otherwise functional, e.g., codon optimized) relative to any such protoparvovirus reference VP1 capsid polypeptide.

Embodiment 2. The construct of embodiment 1, wherein the at least one sequence variation reduces toxicity in a host cell, relative to the protoparvovirus reference VP1 capsid polypeptide.

Embodiment 3. The construct of embodiment 1 or 2, wherein the at least one sequence variation increases virion production in a host cell, relative to the protoparvovirus reference VP1 capsid polypeptide.

Embodiment 4. The construct of any one of the preceding embodiments, wherein the at least one sequence variation increases capsid polypeptide yield, relative to the protoparvovirus reference VP1 capsid polypeptide.

Embodiment 5. The construct of any one of embodiments 2-4, wherein the host cell is an insect cell.

Embodiment 6. The construct of any one of embodiments 2-4, wherein the host cell is a mammalian cell.

Embodiment 7. The construct of any one of the preceding embodiments, wherein the construct comprises a nuclear localization signal (NLS) sequence.

Embodiment 8. The construct of any one of the preceding embodiments, wherein the at least one sequence variation is downstream (e.g., immediately downstream) of the NLS sequence.

Embodiment 9. The construct of any one of the preceding embodiments, wherein the at least one sequence variation is at the 3′ end of the NLS sequence.

Embodiment 10. The construct of any one of the preceding embodiments, wherein the at least one sequence variation comprises a deletion of one or more amino acid residues downstream of the NLS sequence.

Embodiment 11. The construct of any one of the preceding embodiments, wherein the at least one sequence variation comprises a deletion of two or more amino acid residues downstream of the NLS sequence.

Embodiment 12. The construct of any one of the preceding embodiments, wherein the at least one sequence variation comprises a deletion of three or more amino acid residues downstream of the NLS sequence.

Embodiment 13. The construct of any one of the preceding embodiments, wherein the at least one sequence variation comprises a deletion of four or more amino acid residues downstream of the NLS sequence.

Embodiment 14. The construct of any one of the preceding embodiments, wherein the at least one sequence variation comprises a deletion of five or more amino acid residues downstream of the NLS sequence.

Embodiment 15. The construct of any one of the preceding embodiments, wherein the at least one sequence variation comprises a deletion of five or more amino acid residues upstream of a phospholipase A2 (PLA2) motif.

Embodiment 16. The construct of any one of the preceding embodiments, wherein the at least one sequence variation comprises a deletion of five or more amino acid residues between the NLS sequence and the PLA2 motif.

Embodiment 17. The construct of any one of the preceding embodiments, wherein the at least one sequence variation comprises deletion of LVPPG (SEQ ID NO: 1), WVPPG (SEQ ID NO: 2), or WVPPGYNFLG (SEQ ID NO: 3).

Embodiment 18. The construct of any one of the preceding embodiments, wherein the protoparvovirus variant VP1 capsid polypeptide comprises an amino acid sequence with at least 60% identity to SEQ ID NO: 90 (GenBank accession number AXQ00350).

Embodiment 19. The construct of embodiment 18, wherein the protoparvovirus variant VP1 capsid polypeptide comprises an amino acid sequence that is at least about 60% identical to SEQ ID NO: 104 (GenBank accession number AXQ00350).

Embodiment 20. The construct of any one of embodiments 1-17, wherein the at least one sequence variation comprises deletion of residues 12-16 of the protoparvovirus variant VP1 capsid polypeptide.

Embodiment 21. The construct of any one of embodiments 1-17 or 20, wherein the protoparvovirus variant VP1 capsid polypeptide comprises an amino acid sequence with at least 60% identity to SEQ ID NO: 89 (GenBank accession number M19296.1).

Embodiment 22. The construct of any one of embodiments 1-17 or 20, wherein the protoparvovirus variant VP1 capsid polypeptide comprises an amino acid sequence with at least 60% identity to SEQ ID NO: 93 (GenBank accession number ACD37389.1).

Embodiment 23. The construct of any one of embodiments 1-17 or 20, wherein the protoparvovirus variant VP1 capsid polypeptide comprises an amino acid sequence with at least 60% identity to SEQ ID NO: 94 (GenBank accession number AKI88071).

Embodiment 24. The construct of any one of embodiments 1-17 or 20, wherein the protoparvovirus variant VP1 capsid polypeptide comprises an amino acid sequence with at least 60% identity to SEQ ID NO: 95 (GenBank accession number J02275.1).

Embodiment 25. The construct of any one of embodiments 1-17, wherein the at least one sequence variation comprises deletion of residues 10-14 of the protoparvovirus variant VP1 capsid polypeptide.

Embodiment 26. The construct of any one of embodiments 1-17 or 25, wherein the protoparvovirus variant VP1 capsid polypeptide comprises an amino acid sequence with at least 60% identity to SEQ ID NO: 91 (GenBank accession number AQN78782.1).

Embodiment 27. The construct of any one of embodiments 1-17 or 25, wherein the protoparvovirus variant VP1 capsid polypeptide comprises an amino acid sequence with at least 60% identity to SEQ ID NO: 92 (GenBank accession number YP_009508805).

Embodiment 28. The construct of any one of embodiments 1-17 or 25, wherein the protoparvovirus variant VP1 capsid polypeptide comprises an amino acid sequence with at least 60% identity to SEQ ID NO: 88 (GenBank accession number AFN44271).

Embodiment 29. The construct of any one of embodiments 1-17, wherein the at least one sequence variation comprises deletion of residues 11-15 of the protoparvovirus variant VP1 capsid polypeptide.

Embodiment 30. The construct of any one of embodiments 1-17 or 29, wherein the protoparvovirus variant VP1 capsid polypeptide comprises an amino acid sequence with at least 60% identity to SEQ ID NO: 96 (GenBank accession number AIT18930).

Embodiment 31. The construct of any of the preceding embodiments, wherein the at least one sequence variation diminishes human humoral immune response against a virion, and/or reduces neutralization of a virion by human antibodies.

Embodiment 32. The construct of any one of the preceding embodiments, further comprising a sequence that encodes a protoparvovirus VP2 capsid polypeptide.

Embodiment 33. The construct of embodiment 32, wherein the construct includes sequences that direct transcription and/or translation start such that the protoparvovirus VP2 capsid polypeptide is present in excess of the protoparvovirus variant VP1 capsid polypeptide (e.g., wherein the ratio of protoparvovirus VP2 capsid polypeptide to VP1 capsid polypeptide is 25:1, 20:1, 15:1, 10:1, 5:1).

Embodiment 34. The construct of embodiment 33, wherein the VP1 capsid coding sequence comprises fewer translation initiation sequence(s) (e.g., ATG sequence(s)) across the length of the VP1 capsid coding sequence (e.g., in frame or out of frame) that encodes the protoparvovirus variant VP1 capsid polypeptide relative to the reference protoparvovirus VP1 capsid coding sequence.

Embodiment 35. The construct of embodiment 34, wherein the VP1 capsid coding sequence comprises fewer translation initiation sequence(s) (e.g., ATG sequence(s)) across the length of the VP1 capsid coding sequence (e.g., in frame or out of frame) that encodes the protoparvovirus variant VP1 capsid polypeptide due to a deletion in one or more translation initiation sequence(s) relative to the protoparvovirus reference VP1 capsid coding sequence.

Embodiment 36. The construct of embodiment 34, wherein the VP1 capsid coding sequence comprises fewer translation initiation sequence(s) (e.g., ATG sequence(s)) across the length of the VP1 capsid coding sequence (e.g., in frame or out of frame) that encodes the protoparvovirus variant VP1 capsid polypeptide due to a substitution in one more translation initiation sequence(s) relative to the protoparvovirus reference VP1 capsid coding sequence.

Embodiment 37. The construct of embodiment 34, wherein the VP1 capsid coding sequence comprises an alternative translation initiation sequence (e.g., CTG, TTG, ACG, ATC).

Embodiment 38. The construct of embodiment 37, wherein the alternative translation initiation sequence improves potency relative to a construct comprising an ATG initiation sequence.

Embodiment 39. The construct of any one of the preceding embodiments, further comprising a heterologous peptide tag.

Embodiment 40. The construct of embodiment 39, wherein the heterologous peptide tag allows affinity purification using an antibody, an antigen-binding fragment of an antibody, or a nanobody.

Embodiment 41. The construct of embodiment 39 or 40, wherein the heterologous peptide tag comprises an epitope/tag selected from hemagglutinin, His (e.g., 6X-His), FLAG, E-tag, TK15, Strep-tag II, AU1, AU5, Myc, Glu-Glu, KT3, and IRS.

Embodiment 42. The construct of any one of the preceding embodiments, wherein the construct further comprises a nucleic acid sequence that encodes one or more heterologous peptides having a length from about 10 amino acids to 20 amino acids (e.g., according to SEQ ID NOs: 5-84) (e.g., wherein the one or more heterologous peptides comprises or is a heterologous targeting peptide).

Embodiment 43. The construct of embodiment 42, wherein the one or more heterologous peptides are inserted into one or more residues of a protoparvovirus variant VP1 capsid polypeptide corresponding to one or more residues within a variable region of a parvovirus (e.g., AAV) capsid (e.g., wherein the one or more residues of a protoparvovirus variant VP1 capsid polypeptide map(s) onto a structural overlay of one or more residues within a variable region of a parvovirus VP1 capsid (e.g., AAV capsid)).

Embodiment 44. The construct of embodiment 42, wherein the one or more heterologous peptides are inserted into one or more residues along the 3-fold axis of symmetry of a common VP3 region of the protoparvovirus variant VP1 capsid polypeptide.

Embodiment 45. The construct of embodiment 42, wherein the one or more heterologous peptides are inserted into one or more residues along the 3-fold axis of symmetry of a common VP2 region of the protoparvovirus variant VP1 capsid polypeptide.

Embodiment 46. The construct of embodiment 42, wherein the one or more heterologous peptides targets a cell (e.g., a PymT tumor cell, a cervix cancer cell (e.g., a HeLa cell), a K562 cell, a Raji cell, a SKOV-3 cell, a breast cancer cell (e.g., a MCF-7 cell), a M07e cell, a human saphenous vascular endothelial cell (HSaVEC), a MT1-MMP cell, a primary hepatocyte cell (e.g., a Huh7 cell), an immune cell (e.g., a human T cell, e.g., a CD4+ T cell, e.g., a Th2 cell, e.g., a CAR T cell, e.g., a NK cell), a neuron cell (e.g., a LX-2 cell, e.g., a stellate cell, e.g. a primary neuron cell, e.g., neuroblastoma cell (e.g., a SH-SY5Y cell)), a lung cell (e.g., a lung fibroblast cell), a myoblast cell, a myotube cell, a primary cardiomyocyte, a skeletal muscle cell, (e.g., a differentiated skeletal muscle cell), a human vein endothelial cell, a T84 cell, a ileum cell (intestinal), a primary human airway epithelia cell), a kidney cell (e.g., a human renal proximal tubule (HRCE) cell, e.g., a bile duct cell, e.g., an outer medullary cell, e.g., a mixed medullary cell, e.g., renal cortical epithelial cells, e.g., renal epithelial cells), a bone marrow MSC cell, a blood cell (e.g., hematopoietic stem cell (HSC), e.g., a PBMC cell), a small intestine cell, a muscle cell, a heart cell, a spleen cell, a liver cell, a brain cell (e.g., a brain-striatum cell, e.g., a CD105-positive endothelial cell, e.g., a brain cortex cell), an ocular cell, a testes cell, an oocyte, a medulla cell, a striatum cell, a spinal cord (or chord) cell, or a duodenum cell) (e.g., wherein the one or more heterologous peptides comprises or is a heterologous targeting peptide).

Embodiment 47. The construct of any one of the preceding embodiments, wherein the protoparvovirus variant VP1 capsid polypeptide confers increased infectivity, relative to the protoparvovirus reference VP1 capsid polypeptide.

Embodiment 48. The construct of any one of embodiments 42 to 47, wherein the one or more heterologous peptides increases cell specificity and/or viral transduction efficiency and/or increases virion performance.

Embodiment 49. The construct of any one of the preceding embodiments, wherein the expression control sequence comprises a promoter.

Embodiment 50. The construct of embodiment 49, wherein the promoter is a polyhedrin promoter, a P10 promoter, a CMV-b-actin promoter, an OpiE1 promoter, a JeT promoter, a Ubiquitin C promoter, or a truncated CMV enhancer and promoter.

Embodiment 51. The construct of any one of the preceding embodiments, wherein the construct further comprises a 5′ untranslated region (UTR) sequence.

Embodiment 52. The construct of embodiment 51, wherein the 5′ UTR further comprises either a (i) nucleotide spacer sequence or (ii) a Kozak consensus sequence or both.

Embodiment 53. The construct of embodiment 52, wherein the nucleotide spacer sequence comprises a nucleotide sequence according to SEQ ID NO: 121.

Embodiment 54. The construct of embodiment 52, wherein the nucleotide spacer sequence comprises a nucleotide sequence according to SEQ ID NO: 122.

Embodiment 55. The construct of embodiment 52, wherein the Kozak consensus sequence comprises or is a eukaryotic conventional Kozak consensus sequence (GCCGCC - - - G), Viral-derived Kozak consensus sequence (CCTGTTAAG), or alternative Kozak consensus sequence (AAA).

Embodiment 56. The construct of any one of the preceding embodiments, wherein the construct does not comprise a 5′ UTR sequence.

Embodiment 57. The construct of any one of embodiments 1-56, wherein the VP1 capsid coding sequence comprises or is single-stranded deoxyribonucleic acid (ssDNA).

Embodiment 58. The construct of any one of embodiments 1-56, wherein the VP1 capsid coding sequence comprises or is double stranded DNA (dsDNA).

Embodiment 59. The construct of any one of embodiments 1-56, wherein the VP1 capsid coding sequence comprises or is RNA (e.g., an mRNA).

Embodiment 60. The construct of any one of the preceding embodiments, wherein the VP1 capsid coding sequence comprises a sequence according to CTG, TTG, ACG, or ATC.

Embodiment 61. A construct comprising a sequence having at least 70% identity (e.g., 80%, 85%, 90%, 95%, 100% identity) to a sequence shown in Table 4.

Embodiment 62. A protoparvovirus variant VP1 capsid polypeptide having an amino acid sequence that:
(i) shows at least 70% overall sequence identity with that of a protoparvovirus reference VP1 capsid selected from the group consisting of those in Table 3B, which reference polypeptide includes an amino acid sequence element as set forth in SEQ ID NOs: 1-3 or both; and
(ii) includes at least one sequence variation relative to any such protoparvovirus reference VP1 capsid polypeptide.

Embodiment 63. The protoparvovirus variant VP1 capsid polypeptide of embodiment 62, wherein the protoparvovirus variant VP1 capsid polypeptide is characterized by reduced toxicity in a host cell relative to the protoparvovirus reference VP1 capsid polypeptide.

Embodiment 64. The protoparvovirus variant VP1 capsid polypeptide of embodiment 63, wherein the protoparvovirus variant VP1 capsid polypeptide is characterized by improved production of VP1 capsid polypeptide in a host cell relative to the protoparvovirus reference VP1 capsid polypeptide.

Embodiment 65. A virion comprising the protoparvovirus variant VP1 capsid polypeptide of any one of embodiments 1-64.

Embodiment 66. The virion of embodiment 65, wherein the protoparvovirus variant VP1 capsid polypeptide diminishes human humoral immune response against the virion, and/or reduces neutralization of the virion by human antibodies.

Embodiment 67. The virion of embodiment 65 or 66, wherein the protoparvovirus variant VP1 capsid polypeptide increases affinity and/or specificity of the virion to at least one cellular receptor involved in internalization of the virion.

Embodiment 68. The virion of any one of embodiments 65-67, wherein the protoparvovirus variant VP1 capsid polypeptide comprises an insertion of one or more heterologous peptides having a length of from 10 amino acids to 20 amino acids (e.g., wherein the insertion of one or more heterologous peptides is at one or more residues along the 3-fold axis of symmetry of a VP1 capsid polypeptide).

Embodiment 69. The virion of embodiment 68, wherein the one or more heterologous peptides targets a cell (e.g., a PymT tumor cell, a cervix cancer cell (e.g., a HeLa cell), a K562 cell, a Raji cell, a SKOV-3 cell, a breast cancer cell (e.g. a MCF-7 cell), a M07e cell, a human saphenous vascular endothelial cell (HSaVEC), a MT1-MMP cell, a primary hepatocyte cell (e.g., a Huh7 cell), an immune cell (e.g., a human T cell, e.g., a CD4+ T cell, e.g., a Th2 cell, e.g., a CAR T cell, e.g., a NK cell), a neuron cell (e.g., a LX-2 cell, e.g., a stellate cell, e.g. a primary neuron cell, e.g., neuroblastoma cell (e.g., a SH-SY5Y cell), a lung cell (e.g., a lung fibroblast cell), a myoblast cell, a myotube cell, a primary cardiomyocyte, a skeletal muscle cell, (e.g., a differentiated skeletal muscle cell), a human vein endothelial cell, a T84 cell, a ileum cell (intestinal), a primary human airway epithelia cell), a kidney cell (e.g., a human renal proximal tubule (HRCE) cell, e.g., a bile duct cell, e.g., an outer medullary cell, e.g., a mixed medullary cell, e.g., renal cortical epithelial cells, e.g., renal epithelial cells), a bone marrow MSC cell, a blood cell (e.g., hematopoietic stem cell (HSC), e.g., a PBMC cell), a small intestine cell, a muscle cell, a heart cell, a spleen cell, a liver cell, a brain cell (e.g., a brain-striatum cell, e.g., a CD105-positive endothelial cell, e.g., a brain cortex cell), an ocular cell, a testes cell, an oocyte, a medulla cell, a striatum cell, a spinal cord (or chord) cell, or a duodenum cell.

Embodiment 70. The virion of any one of embodiments 65-69, wherein the protoparvovirus variant VP1 capsid polypeptide confers increased infectivity, relative to the protoparvovirus reference VP1 capsid polypeptide.

Embodiment 71. The virion of any one of embodiments 65-70, wherein the one or more heterologous peptides increases cell specificity and/or viral transduction efficiency and/or increases virion performance.

Embodiment 72. The virion of any one of embodiments 65-71, further comprising a heterologous nucleic acid sequence.

Embodiment 73. The virion of embodiment 72, wherein the heterologous nucleic acid comprises a nucleic acid sequence that is at least about 60% identical to a nucleic acid sequence of a target cell.

Embodiment 74. The virion of embodiment 72 or 73, wherein the heterologous nucleic acid is at least about 60% identical to a nucleic acid of a mammal, preferably wherein the mammal is a human.

Embodiment 75. The virion of any one of embodiments 72-74, wherein the heterologous nucleic acid sequence comprises at least one inverted terminal repeat (ITR).

Embodiment 76. The virion of embodiment 75, wherein the at least one ITR comprises one or more of the following:
(a) a dependoparvovirus ITR,
(b) a bocaparvovirus ITR
(c) a protoparvovirus ITR,
(d) a tetraparvovirus ITR, or
(e) an erthythroparvovirus ITR.

Embodiment 77. The virion of any one of embodiments 72-76, wherein the heterologous nucleic acid sequence is deoxyribonucleic acid (DNA).

Embodiment 78. The virion of embodiment 77, wherein the DNA is single-stranded or self-complementary duplex.

Embodiment 79. The virion of any one of embodiments 72-78, wherein the heterologous nucleic acid sequence comprises a Rep protein-dependent origin of replication (ori).

Embodiment 80. The virion of any one of embodiments 72-79, wherein the heterologous nucleic acid sequence comprises a transgene coding sequence.

Embodiment 81. The virion of any one of embodiments 72-80, wherein the transgene coding sequence is operably linked to a transgene promoter, optionally placed between two ITRs.

Embodiment 82. The virion of embodiment 80, wherein the transgene coding sequences comprises one or more of:
(a) a gene encoding a protein or a fragment thereof, preferably a human protein or a fragment thereof;
(b) a nucleic acid encoding a nuclease, optionally a Transcription Activator-Like Effector Nuclease (TALEN), a zinc-finger nuclease (ZFN), a meganuclease, a megaTAL, or a CRISPR endonuclease, (e.g., a Cas9 endonuclease or a variant thereof);
(c) a nucleic acid encoding a reporter, e.g., luciferase or GFP; or
(d) a nucleic acid encoding a drug resistance protein, e.g., neomycin resistance.

Embodiment 83. The virion of embodiment 81 or 82, wherein the transgene coding sequence is codon-optimized for expression in a target cell.

Embodiment 84. The virion of embodiment 83, wherein the target cell is or comprises a PymT tumor cell, a cervix cancer cell (e.g., a HeLa cell), a K562 cell, a Raji cell, a SKOV-3 cell, a breast cancer cell (e.g. a MCF-7 cell), a M07e cell, a human saphenous vascular endothelial cell (HSaVEC), a MT1-MMP cell, a primary hepatocyte cell (e.g., a Huh7 cell), an immune cell (e.g., a human T cell (e.g., a CD4+ T cell, e.g., a Th2 cell, e.g., a CAR T cell), e.g., a NK cell), a neuron cell (e.g., a LX-2 cell, e.g., a stellate cell, e.g. a primary neuron cell, e.g., neuroblastoma cell (e.g., a SH-SY5Y cell)), a human vein endothelial cell, a T84 cell, a ileum cell (intestinal), a primary human airway epithelia cell, a kidney cell (e.g., a human renal proximal tubule (HRCE) cell, e.g., a bile duct cell, e.g., an outer medullary cell, e.g., a mixed medullary cell, e.g., renal cortical epithelial cells, e.g., renal epithelial cells), a bone marrow MSC cell, a blood cell (e.g., hematopoietic stem cell (HSC)), a small intestine cell, a spleen cell, a liver cell, a heart cell (e.g., a myoblast cell, e.g., a myotube cell, e.g., a primary cardiomyocyte), a lung cell (e.g., a lung fibroblast cell), a brain cell (e.g., a brain-striatum cell, e.g., CD105-positive endothelial cells, e.g., a brain cortex cell), a muscle cell (e.g., a skeletal muscle cell, e.g., a differentiated skeletal muscle cell), a testes cell, an oocyte, a medulla cell, a striatum cell, a spinal cord (or chord) cell, or a duodenum cell).

Embodiment 85. The virion of any one of embodiments 80-84, wherein the transgene coding sequence comprises a hemoglobin gene (HBA1, HBA2, HBB, HBG1, HBG2, HBD, HBE1, and/or HBZ), a gene encoding an alpha-hemoglobin stabilizing protein (AHSP), coagulation factor VIII, coagulation factor IX, von Willebrand factor, dystrophin or truncated dystrophin, micro-dystrophin, utrophin or truncated utrophin, micro-utrophin, usherin (USH2A), CEP290, glial cell line-derived neurotrophic factor (GDNF), neuturin (NTN), HTT, neuronal apoptosis inhibitory protein (NAIP), cystic fibrosis transmembrane conductance regulator (CFTR), F8 or a fragment thereof (e.g., fragment encoding B-domain deleted polypeptide (e.g., VIII SQ, p-VIII)), T cell receptor (e.g., TCR alpha or TCR beta), a gene associated with lysosomal storage diseases, a gene associated with Alport syndrome (e.g., Col4a3, Col4a4, Col4a5), a gene associated with Fabry disease (e.g., GLA), a gene associated with autosomal dominant polycystic kidney disease (PKD) (e.g., PKD, PKD1, PKD2), a gene associated with congenital nephrotic syndrome (e.g., NPHS1 (Nephrin), NPHS2 (Podocin), a gene associated with hypertrophic cardiomyopathy (e.g., MYBPC3, JPH2, ALPK3), a gene associated with dilated cardiomyopathy (e.g., RBM20), or a gene associated with dilated cardiomyopathy (e.g., ALPK3, LMNA, BAG3).

Embodiment 86. The virion of any one of embodiments 72-85, wherein the heterologous nucleic acid sequence comprises a non-coding sequence.

Embodiment 87. The virion of embodiment 86, wherein the non-coding sequence comprises or is RNA.

Embodiment 88. The virion of embodiment 87, wherein the RNA comprises or is lncRNA, miRNA, shRNA, siRNA, antisense RNA, and/or guide RNA.

Embodiment 89. The virion of embodiment 86, wherein the non-coding sequence comprises or is DNA.

Embodiment 90. The virion of embodiment 89, wherein the DNA comprises or is:
(a) a transcription regulatory element (e.g., an enhancer, a transcription termination sequence, an untranslated region (5′ or 3′ UTR), a proximal promoter element, a locus control region, a polyadenylation signal sequence), and/or
(b) a translation regulatory element (e.g., Kozak sequence, woodchuck hepatitis virus post-transcriptional regulatory element).

Embodiment 91. The virion of embodiment 90, wherein the DNA comprises or is a transcription regulatory element, and wherein the transcription regulatory element is a locus control region, optionally a β-globin LCR or a DNase hypersensitive site (HS) of β-globin LCR.

Embodiment 92. The virion of any one of embodiments 80-91, wherein the transgene coding sequence (or the protein translated therefrom) or the non-coding sequence increases or restores the expression of an endogenous gene of the target cell.

Embodiment 93. The virion of any one of embodiments 80-91, wherein the transgene coding sequence (or the protein translated therefrom) or the non-coding sequence decreases or eliminates the expression of an endogenous gene of the target cell.

Embodiment 94. The virion of any one of embodiments 81-93, wherein the transgene promoter is selected from:
(a) a promoter heterologous to a nucleic acid;
(b) a promoter that facilitates the tissue-specific expression of a nucleic acid, preferably wherein the transgene promoter facilitates hematopoietic cell-specific expression or erythroid lineage-specific expression;
(c) a promoter that facilitates the constitutive expression of a nucleic acid; and
(d) a promoter that is inducibly expressed, optionally in response to a metabolite or small molecule or chemical entity.

Embodiment 95. The virion of any one of embodiments 81-94, wherein the transgene promoter is selected from the CMV promoter, β-globin promoter, CAG promoter, AHSP promoter, MND promoter, Wiskott-Aldrich promoter, and PKLR promoter.

Embodiment 96. The virion of any one of embodiments 65-95, wherein the virion is icosahedral.

Embodiment 97. The virion of any one of embodiments 65-95, wherein the protoparvovirus variant VP1 capsid polypeptide is phosphorylated.

Embodiment 98. A population of virions according to any one of embodiments 65-97, wherein the population is characterized as having reduced toxicity in a host cell, improved virion production in a host cell, increased capsid polypeptide yield, or any combination thereof, relative to a population of virions comprising the protoparvovirus reference VP1 capsid polypeptide.

Embodiment 99. A system comprising a construct of any one of embodiments 1-61 and/or a second construct comprising a sequence that encodes a protoparvovirus VP2 capsid polypeptide, wherein the protoparvovirus VP2 capsid polypeptide is present in excess of the protoparvovirus variant VP1 capsid polypeptide (e.g., wherein the ratio of protoparvovirus VP2 capsid polypeptide to VP1 capsid polypeptide is 25:1, 20:1, 15:1, 10:1, 5:1).

Embodiment 100. A system comprising a protoparvovirus variant VP1 capsid polypeptide of any one of embodiments 62-64 and a protoparvovirus VP2 capsid polypeptide, wherein the protoparvovirus VP2 capsid polypeptide is present in excess of the protoparvovirus variant VP1 capsid polypeptide (e.g., wherein the ratio of protoparvovirus VP2 capsid polypeptide to VP1 capsid polypeptide is 25:1, 20:1, 15:1, 10:1, 5:1).

Embodiment 101. A composition comprising a construct of any one of embodiments 1-61.

Embodiment 102. A composition comprising a virion of any one of embodiments 65-97.

Embodiment 103. A composition comprising a population of virions of embodiment 98.

Embodiment 104. A composition comprising a protoparvovirus variant VP1 capsid polypeptide of any one of embodiments 62-64.

Embodiment 105. The composition of any one of embodiments 101-104, wherein the composition is a pharmaceutical composition.

Embodiment 106. The composition of embodiment 105, further comprising a pharmaceutically acceptable carrier.

Embodiment 107. A kit comprising a construct of any one of embodiments 1-61 and a construct comprising a codon sequence encoding a least one capsid replication protein (e.g., NS1) of a protoparvovirus operably linked to an expression control sequence for expression in a host cell.

Embodiment 108. A host cell comprising a construct of any one of embodiments 1-61.

Embodiment 109. A host cell comprising a protoparvovirus variant VP1 capsid polypeptide of any one of embodiments 62-64.

Embodiment 110. A host cell comprising a virion of any one of embodiments 65-97.

Embodiment 111. A host cell comprising a population of virions of embodiment 98.

Embodiment 112. A host cell comprising a composition of any one of embodiments 101-106.

Embodiment 113. The host cell of embodiment 108, further comprising a second construct comprising a polynucleotide comprising at least one ITR nucleotide sequence.

Embodiment 114. The host cell of embodiment 113, wherein the at least one ITR comprises a parvovirus ITR.

Embodiment 115. The host cell of embodiment 113, wherein the at least one ITR comprises one or more of the following:
(a) a dependoparvovirus ITR,
(b) a bocaparvovirus ITR
(c) a protoparvovirus ITR,
(d) a tetraparvovirus ITR, or
(e) an erthythroparvovirus ITR.

Embodiment 116. The host cell of embodiment 115, wherein the at least one ITR comprises a dependoparvovirus ITR, wherein the at least one dependoparvovirus ITR comprises an AAV ITR, optionally an AAV2 ITR.

Embodiment 117. The host cell of any one of claims 108-116, further comprising a third construct comprising a polynucleotide comprising:
(1) at least one capsid replication protein (e.g., NS1) of a protoparvovirus operably linked to an expression control sequence for expression in a host cell,
(2) (i) at least one ITR replication protein of a protoparvovirus, bocaparvovirus, dependoparvovirus, tetraparvovirus, or erythroparvovirus, or (ii) at least one ITR replication protein of an AAV, optionally wherein the at least one ITR replication protein of an AAV comprises (a) a Rep52 or a Rep40 coding sequence operably linked to an expression control sequence for expression in a host cell, and/or (b) a Rep78 or a Rep68 coding sequence operably linked to an expression control sequence for expression in a host cell, or
(3) a combination of (1) and (2i) or (1) and (2ii).

Embodiment 118. The host cell of any one of embodiments 108-117, wherein at least the first construct, the second construct, or the third construct is stably integrated in the host cell genome.

Embodiment 119. The host cell of any one of embodiments 108-118, wherein the at least one capsid replication protein of a protoparvovirus is an NS1 protein (e.g., having at least 30% identity to SEQ ID NO: 4).

Embodiment 120. The host cell of any one of embodiments 108-118, wherein the host cell is an insect cell.

Embodiment 121. The host cell of any one of embodiments 108-118, wherein the host cell is a mammalian cell.

Embodiment 122. The host cell of embodiment 120, wherein the host cell is derived from a species of lepidoptera.

Embodiment 123. The host cell of embodiment 122, wherein the species of lepidoptera is Spodoptera frugiperda, Spodoptera littoralis, Spodoptera exigua, or Trichoplusiani.

Embodiment 124. The host cell of embodiment 120, wherein the insect cell is Sf9.

Embodiment 125. The host cell of any one of embodiments 111-124, wherein the construct is a baculoviral construct, a viral construct, or a plasmid.

Embodiment 126. The host cell of any one of embodiments 111-125, wherein the construct is a baculoviral construct.

Embodiment 127. The host cell of any one of embodiments 113-126, wherein the expression control sequence for expression in a host cell comprises a promoter.

Embodiment 128. The host cell of embodiment 127, wherein the promoter comprises:
(a) an immediate early promoter of an animal DNA virus,
(b) an immediate early promoter of a host virus, or
(c) a host cell promoter.

Embodiment 129. The host cell of embodiment 128, wherein the animal DNA virus is cytomegalovirus (CMV), parvovirus, or AAV.

Embodiment 130. The host cell of embodiment 128, wherein the host virus is a lepidopteran virus or a baculovirus, optionally wherein the baculovirus is Autographa californica multicapsid nucleopolyhedrovirus (AcMNPV).

Embodiment 131. The host cell of any one of embodiments 127-130, wherein the promoter is a polyhedrin (polh) promoter, a Immediately early 1 gene (IE-1) promoter, a P10 promoter, a CMV-b-actin promoter, an OpiE1 promoter, a JeT promoter, a Ubiquitin C promoter, or a truncated CMV enhancer and promoter.

Embodiment 132. The host cell of any one of embodiments 113-131, wherein the heterologous nucleic acid sequence comprises at least one ITR replication protein of an AAV comprises a nucleotide sequence encoding Rep52 and/or Rep78.

Embodiment 133. The host cell of any one of embodiments 113-131, wherein the AAV is AAV2.

Embodiment 134. A method of producing a virion according to any one of embodiments 65-91 or a population of virions according to embodiment 98, comprising:
(1) providing one or more of the following:
(i) a first construct comprising at least one ITR nucleotide sequence, optionally further comprising a heterologous nucleic acid operably linked to a promoter for expression in a target cell,
(ii) a second construct comprising a construct according to any one of embodiments 1-61 and/or a construct comprising a VP1 capsid coding sequence linked to an expression control sequence, wherein the VP1 capsid coding sequence encodes a protoparvovirus variant VP1 capsid polypeptide, wherein the expression control sequence comprises or is an expression control sequence for expression in a host cell, and
(2) introducing the first construct and/or the second construct into a host cell, and
(3) maintaining said host cell under conditions such that a virion according to any one of embodiments 65-97 or a population of virions according to embodiment 98 is produced.

Embodiment 135. The method of embodiment 134, further comprising (4) providing a third construct comprising:
(A) at least one capsid replication protein (e.g., NS1) of protoparvovirus operably linked to an expression control sequence for expression in a host cell (e.g., wherein the at least one capsid replication protein of a protoparvovirus enhances encapsidation, relative to encapsidation without the at least one capsid replication protein of a protoparvovirus),
(B) at least one ITR replication protein of an AAV, optionally wherein the at least one ITR replication protein of an AAV comprises (a) a Rep52 or a Rep40 coding sequence operably linked to an expression control sequence for expression in a host cell, and/or (b) a Rep78 or a Rep68 coding sequence operably linked to an expression control sequence for expression in a host cell, or
(C) a combination of (A) and (B).

Embodiment 136. The method of embodiment 134, wherein the host cell achieves a cell viability of greater than 50% (e.g., of greater than 60%, 70%, or 80%).

Embodiment 137. A method of producing a virion according to any one of embodiments 65-97 or a population of virions according to embodiment 98 in a host cell, the method comprising:
(1) providing a host cell comprising
(i) a first construct comprising at least one ITR nucleotide sequence, optionally further comprising a heterologous nucleic acid operably linked to a promoter for expression in a target cell,
(ii) a second construct comprising a construct according to any one of embodiments 1-65 and/or a construct comprising a VP1 capsid coding sequence linked to an expression control sequence, wherein the VP1 capsid coding sequence encodes a protoparvovirus variant VP1 capsid polypeptide, wherein the expression control sequence comprises or is an expression control sequence for expression in a host cell, and
(iii) a third construct comprising
(A) at least one capsid replication protein (e.g., NS1) of protoparvovirus operably linked to an expression control sequence for expression in a host cell (e.g., wherein the at least one capsid replication protein of a protoparvovirus enhances encapsidation, relative to encapsidation without the at least one capsid replication protein of a protoparvovirus),
(B) at least one ITR replication protein of an AAV, optionally wherein the at least one ITR replication protein of an AAV comprises (a) a Rep52 or a Rep40 coding sequence operably linked to an expression control sequence for expression in a host cell, and/or (b) a Rep78 or a Rep68 coding sequence operably linked to an expression control sequence for expression in a host cell, or
(C) a combination of (A) and (B),
optionally, a fourth construct,
wherein at least one of (i), (ii), (iii) (A), (iii) (B), and (iii) (C) is/are stably integrated in the host cell genome, and the fourth construct, when present, comprises the remainder of the (i), (ii), (iii) (A), (iii) (B), and (iii) (C) nucleotide sequences which is/are not stably integrated in the host cell genome, and
(2) maintaining the host cell under conditions such that a virion according to any one of embodiments 65-97 or a population of virions according to embodiment 98 is produced.

Embodiment 138. The method of embodiment 137, wherein the host cell achieves a cell viability of greater than 50% (e.g., of greater than 60%, 70%, or 80%).

Embodiment 139. The method of any one of embodiment 137 or 138, wherein the host cell is derived from a species of lepidoptera.

Embodiment 140. The method of embodiment 139, wherein the species of lepidoptera is Spodoptera frugiperda, Spodoptera littoralis, Spodoptera exigua, or Trichoplusiani.

Embodiment 142. The method of any one of embodiments 137-139, wherein the host cell is Sf9.

Embodiment 143. The method of embodiment 137 or 138, wherein the host cell is a mammalian cell.

Embodiment 144. The method of any one of embodiments 137-143, wherein the at least one construct is a baculoviral construct, a viral construct, or a plasmid.

Embodiment 145. The method of any one of embodiments 137-144, wherein the at least one construct is a baculoviral construct.

Embodiment 146. The method of any one of embodiments 137-145, wherein the at least one ITR comprises one or more of the following:
(a) a dependoparvovirus ITR,
(b) a bocaparvovirus ITR
(c) a protoparvovirus ITR,
(d) a tetraparvovirus ITR, or
(e) an erthythroparvovirus ITR.

Embodiment 147. The method of any one of embodiments 137-146, wherein the expression control sequence for expression in a host cell comprises:
(a) a promoter, and/or
(b) a Kozak consensus sequence.

Embodiment 148. The method of any one of embodiments 137-147, wherein the nucleotide sequence comprising at least one ITR replication protein of an AAV comprises a nucleotide sequence encoding Rep52 and/or Rep78.

Embodiment 149. The method of any one of embodiments 137-148, wherein the AAV is AAV2.

Embodiment 150. A method of purifying a virion according to any one of embodiments 65-97 or a population of virions according to embodiment 98, wherein the virion or the population of virions is purified using an antibody, an antigen-binding fragment of an antibody, or a nanobody that binds the virion.

Embodiment 151. The method of embodiment 150, wherein the antibody, an antigen-binding fragment of an antibody, or a nanobody binds the heterologous peptide tag in the capsid of the virion.

Embodiment 152. The method of embodiment 151, wherein the heterologous peptide tag comprises an epitope/tag selected from hemagglutinin, His (e.g., 6X-His), FLAG, E-tag, TK15, Strep-tag II, AU1, AU5, Myc, Glu-Glu, KT3, and IRS.

Embodiment 153. A method of preventing or treating a disease, comprising: administering to a subject in need thereof an effective amount of virion according to any one of embodiments 65-97 or a population of virions according to embodiment 98 or a pharmaceutical composition of embodiment 105.

Embodiment 154. A method of preventing or treating a disease, comprising:
(a) obtaining a plurality of cells;
(b) transducing the cells with a virion according to any one of embodiments 65-97 or a population of virions according to embodiment 98 or the pharmaceutical composition of embodiment 105, optionally further selecting or screening for the transduced cells; and
(c) administering an effective amount of the transduced cells to a subject in need thereof.

Embodiment 155. The method of embodiments 153 and 154, further comprising co-administering an immune suppressant and/or a prophylactic to mitigate an immune response.

Embodiment 156. A method of characterizing a virion according to any one of embodiments 65-97 or a population of virions according to embodiment 98 or the pharmaceutical composition of embodiment 105.

Embodiment 157. A method of manufacturing an intermediate (e.g., any intermediate that can be stored or shipped) of a virion according to any one of embodiments 65-97 or a population of virions according to embodiment 98 or the pharmaceutical composition of embodiment 105.

Embodiment 158. A method of providing a virion according to any one of embodiments 65-97 or a population of virions according to embodiment 98 or the pharmaceutical composition of embodiment 105, comprising assessing one or more characteristics of the virion or the population of virions and establishing one or more characteristics of the virion or population of virions (e.g., compared to a reference sample).

Embodiment 159. A system comprising a host cell according to any one of embodiments 108-133.

Embodiment 160. A method comprising contacting a cell with a construct of any one of embodiments 1-61.

Embodiment 161. A virion according to any one of embodiments 65-97 or a population of virions according to embodiment 98 or the pharmaceutical composition of embodiment 105 for use in the treatment of a disease or disorder.

Embodiment 162. Use of a construct of any one of embodiments 1-61 for the manufacture of a medicament to treat a disease or disorder.

Embodiment 163. Use of a virion of any one of embodiments 65-97 for the manufacture of a medicament to treat a disease or disorder.

Embodiment 164. Use of a population of virions of embodiment 98 for the manufacture of a medicament to treat a disease or disorder.

Embodiment 165. A kit comprising a construct of any one of embodiments 1-61, a protoparvovirus variant VP1 capsid polypeptide of any one of embodiments 62-64, a virion of any one of embodiments 65-97, a population of virions of embodiment 98, a composition of any one of embodiments 101-106, or a host cell of any one of embodiments 108-133.

### EQUIVALENTS

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

It is to be understood that while the disclosure has been described in conjunction with the detailed description thereof, the foregoing description is intended to illustrate and not limit the scope of the present disclosure, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A construct comprising a VP1 capsid coding sequence operably linked to an expression control sequence, wherein the VP1 capsid coding sequence encodes a protoparvovirus variant VP1 capsid polypeptide having an amino acid sequence that:
(i) shows at least 70% overall sequence identity with that of a protoparvovirus reference VP1 capsid polypeptide selected from the group consisting of those in Table 3B, which reference polypeptide includes an amino acid sequence element as set forth in SEQ ID NOs: 1-3; and
(ii) includes at least one sequence variation relative to SEQ ID NOs: 1-3 in any such protoparvovirus reference VP1 capsid polypeptide.

2.-6. (canceled)

7. The construct of claim 1, wherein the construct comprises a nuclear localization signal (NLS) sequence.

8. The construct of claim 1, wherein the at least one sequence variation is downstream of a NLS sequence.

9. (canceled)

10. The construct of claim 1, wherein the at least one sequence variation comprises a deletion of one or more amino acid residues downstream of a NLS sequence.

11.-14. (canceled)

15. The construct of claim 1, wherein the at least one sequence variation comprises a deletion of one or more amino acid residues upstream of a phospholipase A2 (PLA2) motif.

16. The construct of claim 15, wherein the at least one sequence variation comprises a deletion of one or more amino acid residues between the NLS sequence and the PLA2 motif.

17. The construct of claim 1, wherein the at least one sequence variation comprises deletion of LVPPG (SEQ ID NO: 1), WVPPG (SEQ ID NO: 2), or WVPPGYNFLG (SEQ ID NO: 3).

18.-31. (canceled)

32. The construct of claim 1, further comprising a sequence that encodes a protoparvovirus VP2 capsid polypeptide.

33. The construct of claim 32, wherein the construct includes sequences that direct transcription and/or translation start such that the protoparvovirus VP2 capsid polypeptide is present in excess of the protoparvovirus variant VP1 capsid polypeptide.

34. The construct of claim 33, wherein the VP1 capsid coding sequence comprises fewer translation initiation sequence(s) across the length of the VP1 capsid coding sequence that encodes the protoparvovirus variant VP1 capsid polypeptide relative to the reference protoparvovirus VP1 capsid coding sequence.

35.-41. (canceled)

42. The construct of claim 1, wherein the construct further comprises a nucleic acid sequence that encodes one or more heterologous peptides having a length from about 10 amino acids to 20 amino acids.

43.-48. (canceled)

49. The construct of claim 1, wherein the expression control sequence comprises a promoter.

50. (canceled)

51. The construct of claim 1, wherein the construct further comprises a 5′ untranslated region (UTR) sequence.

52. The construct of claim 51, wherein the 5′ UTR further comprises either a (i) nucleotide spacer sequence or (ii) a Kozak consensus sequence or both.

53.-60. (canceled)

61. A nucleic acid construct comprising a sequence that encodes a protoparvovirus variant VP1 capsid polypeptide, wherein the protoparvovirus variant VP1 capsid polypeptide has an amino acid sequence having at least 70% identity to any one of SEQ ID NOs: 104-106.

62. A protoparvovirus variant VP1 capsid polypeptide having an amino acid sequence that:
(i) shows at least 70% overall sequence identity with that of a protoparvovirus reference VP1 capsid selected from the group consisting of those in Table 3B, which reference polypeptide includes an amino acid sequence element as set forth in SEQ ID NOs: 1-3; and
(ii) includes at least one sequence variation relative to SEQ ID NOs: 1-3 in any such protoparvovirus reference VP1 capsid polypeptide.

63.-64. (canceled)

65. A virion comprising:
(1) a protoparvovirus variant VP1 capsid polypeptide of having an amino acid sequence that:
(i) shows at least 70% overall sequence identity with that of a protoparvovirus reference VP1 capsid selected from the group consisting of those in Table 3B, which reference polypeptide includes an amino acid sequence element as set forth in SEQ ID NOs: 1-3, and
(ii) includes at least one sequence variation relative to SEQ ID NOs: 1-3 in any such protoparvovirus reference VP1 capsid polypeptide; and
(2) a heterologous nucleic acid sequence comprising:
(i) a transgene coding sequence, and
(ii) at least one inverted terminal repeat (ITR).

66.-67. (canceled)

68. The virion of claim 65, wherein the protoparvovirus variant VP1 capsid polypeptide comprises an insertion of one or more heterologous peptides having a length of from 10 amino acids to 20 amino acids.

69.-80. (canceled)

81. The virion of claim 65, wherein the transgene coding sequence is operably linked to a transgene promoter, optionally placed between two ITRs.

82.-96. (canceled)

97. The virion of claim 65, wherein the protoparvovirus variant VP1 capsid polypeptide is phosphorylated.

98.-101. (canceled)

102. A composition comprising the virion of claim 65.

103.-104. (canceled)

105. The composition of claim 102, wherein the composition is a pharmaceutical composition.

106. (canceled)

107. A kit comprising the construct of claim 1 and a construct comprising a coding sequence encoding a least one capsid replication protein of a protoparvovirus operably linked to an expression control sequence for expression in a host cell.

108.-109. (canceled)

110. A host cell comprising the virion of claim 65.

111.-133. (canceled)

134. A method of producing the virion according to claim 65, comprising:
(1) providing one or more of the following:
(i) a first construct comprising at least one ITR nucleotide sequence, optionally further comprising a heterologous nucleic acid operably linked to a promoter for expression in a target cell,
(ii) a second construct comprising a construct comprising a VP1 capsid coding sequence operably linked to an expression control sequence, wherein the VP1 capsid coding sequence encodes a protoparvovirus variant VP1 capsid polypeptide having an amino acid sequence that:
(a) shows at least 70% overall sequence identity with that of a protoparvovirus reference VP1 capsid polypeptide selected from the group consisting of those in Table 3B, which reference polypeptide includes an amino acid sequence element as set forth in SEQ ID NOs: 1-3; and
(b) includes at least one sequence variation relative to SEQ ID NOs: 1-3 in any such protoparvovirus reference VP1 capsid polypeptide; and
(2) introducing the first construct and/or the second construct into a host cell, and
(3) maintaining said host cell under conditions such that the virion is produced.

135. The method of claim 134, further comprising (4) providing a third construct comprising:
(A) at least one capsid replication protein of protoparvovirus operably linked to an expression control sequence for expression in a host cell,
(B) at least one ITR replication protein of an AAV, optionally wherein the at least one ITR replication protein of an AAV comprises (a) a Rep52 or a Rep40 coding sequence operably linked to an expression control sequence for expression in a host cell, and/or (b) a Rep78 or a Rep68 coding sequence operably linked to an expression control sequence for expression in a host cell, or
(C) a combination of (A) and (B).

136.-152. (canceled)

153. A method of preventing or treating a disease, comprising: administering to a subject in need thereof an effective amount of virion according to claim 65.

154.-165. (canceled)


SEQUENCE LISTING (221 sequences)

SEQ ID NO: 1 - protein, 5 aa, synthetic construct
LVPPG

SEQ ID NO: 2 - protein, 5 aa, synthetic construct
WVPPG

SEQ ID NO: 3 - protein, 10 aa, synthetic construct
WVPPGYNFLG

SEQ ID NO: 4 - protein, 668 aa, synthetic construct
MSGNQYTEEVMEGVNWLKKHAENEAFSFVFKCDNVQLNGKDVRWNNYTKPIQNEELTSLIRGAQTAMDQTEEEEMDWESEVDSLAKKQVQTFDALIKKCLFEVFVSKNIEPNECVWFIQHEWGKDQGWHCHVLLHSKNLQQATGKWLRRQMNMYWSRWLVTLCSVNLTPTEKIKLREIAEDSEWVTILTYRHKQTKKDYVKMVHFGNMIAYYFLTKKKIVHMTKESGYFLSTDSGWKFNFMKYQDRQIVSTLYTEQMKPETVETTVTTAQETKRGRIQTKKEVSIKCTLRDLVSKRVTSPEDWMMLQPDSYIEMMAQPGGENLLKNTLEICTLTLARTKTAFELILEKADNTKLTNFDLANSRTCQIFRMHGWNWIKVCHAIACVLNRQGGKRNTVLFHGPASTGKSIIAQAIAQAVGNVGCYNAANVNFPFNDCTNKNLIWIEEAGNFGQQVNQFKAICSGQTIRIDQKGKGSKQIEPTPVIMTTNENITIVRIGCEERPEHTQPIRDRMLNIKLVCKLPGDFGLVDKEEWPLICAWLVKHGFVSTMANYTHHWGKVPEWDENWAEPKIQEGINSPGCKDLKTQAASNPQSQDQVLTPLTPDVVDLALEPWSTPDTPIAETANQQSNQLGVTHKDVQASPTWSEIEADLRAIFTSEQLEEDFRDDLD

SEQ ID NO: 5 - protein, 14 aa, synthetic construct
QAGTFALRGDNPQG

SEQ ID NO: 6 - protein, 6 aa, synthetic construct
NGRAHA

SEQ ID NO: 7 - protein, 7 aa, synthetic construct
RGDAVGV

SEQ ID NO: 8 - protein, 7 aa, synthetic construct
RGDTPTS

SEQ ID NO: 9 - protein, 7 aa, synthetic construct
GENQARS

SEQ ID NO: 10 - protein, 7 aa, synthetic construct
RSNAVVP

SEQ ID NO: 11 - protein, 9 aa, synthetic construct
CDCRGDCFC

SEQ ID NO: 12 - protein, 7 aa, synthetic construct
PRGTNGP

SEQ ID NO: 13 - protein, 7 aa, synthetic construct
SRGATTT

SEQ ID NO: 14 - protein, 7 aa, synthetic construct
SIGYPLP

SEQ ID NO: 15 - protein, 12 aa, synthetic construct
MTPFPTSNEANL

SEQ ID NO: 16 - protein, 7 aa, synthetic construct
QPEHSST

SEQ ID NO: 17 - protein, 7 aa, synthetic construct
VNTANST

SEQ ID NO: 18 - protein, 9 aa, synthetic construct
CNHRYMQMC

SEQ ID NO: 19 - protein, 9 aa, synthetic construct
CAPGPSKSG

SEQ ID NO: 20 - protein, 7 aa, synthetic construct
EYHHYNK

SEQ ID NO: 21 - protein, 7 aa, synthetic construct
ASSLNIA

SEQ ID NO: 22 - protein, 7 aa, synthetic construct
TQVGQKT

SEQ ID NO: 23 - protein, 7 aa, synthetic construct
LPSSLQK

SEQ ID NO: 24 - protein, 7 aa, synthetic construct
WPFYGTP

SEQ ID NO: 25 - protein, 7 aa, synthetic construct
DSPAHPS

SEQ ID NO: 26 - protein, 7 aa, synthetic construct
GWTLHNK

SEQ ID NO: 27 - protein, 7 aa, synthetic construct
GMNAFRA

SEQ ID NO: 28 - protein, 7 aa, synthetic construct
LGETTRP

SEQ ID NO: 29 - protein, 7 aa, synthetic construct
RGDTATL

SEQ ID NO: 30 - protein, 7 aa, synthetic construct
PRGDLAP

SEQ ID NO: 31 - protein, 7 aa, synthetic construct
RGDQQSL

SEQ ID NO: 32 - protein, 10 aa, synthetic construct
EQLSISEEDL

SEQ ID NO: 33 - protein, 35 aa, synthetic construct
  VARIANT 35: note = X can be any amino acid
FNMQCQRRFYEALHDPNLNEEQRNAKIKSIRDDCX

SEQ ID NO: 34 - protein, 15 aa, synthetic construct
GLNDIFEAQKIEWHE

SEQ ID NO: 35 - protein, 13 aa, synthetic construct
LCTPSRAALLTGR

SEQ ID NO: 36 - protein, 15 aa, synthetic construct
QVSHWVSGLAEGSFG

SEQ ID NO: 37 - protein, 15 aa, synthetic construct
LSHTSGRVEGSVSLL

SEQ ID NO: 38 - protein, 7 aa, synthetic construct
VTAGRAP

SEQ ID NO: 39 - protein, 7 aa, synthetic construct
APVTRPA

SEQ ID NO: 40 - protein, 7 aa, synthetic construct
DLSNLTR

SEQ ID NO: 41 - protein, 7 aa, synthetic construct
NQVGSWS

SEQ ID NO: 42 - protein, 7 aa, synthetic construct
EARVRPP

SEQ ID NO: 43 - protein, 7 aa, synthetic construct
NSVSLYT

SEQ ID NO: 44 - protein, 7 aa, synthetic construct
NDVRSAN

SEQ ID NO: 45 - protein, 7 aa, synthetic construct
NESRVLS

SEQ ID NO: 46 - protein, 7 aa, synthetic construct
NRTWEQQ

SEQ ID NO: 47 - protein, 7 aa, synthetic construct
NSVQSSW

SEQ ID NO: 48 - protein, 7 aa, synthetic construct
RGDLGLS

SEQ ID NO: 49 - protein, 7 aa, synthetic construct
RGDMSRE

SEQ ID NO: 50 - protein, 7 aa, synthetic construct
ESGLSQS

SEQ ID NO: 51 - protein, 7 aa, synthetic construct
EYRDSSG

SEQ ID NO: 52 - protein, 7 aa, synthetic construct
DLGSARA

SEQ ID NO: 53 - protein, 7 aa, synthetic construct
GPQGKNS

SEQ ID NO: 54 - protein, 7 aa, synthetic construct
NSSRDLG

SEQ ID NO: 55 - protein, 7 aa, synthetic construct
NDVRAVS

SEQ ID NO: 56 - protein, 7 aa, synthetic construct
PRSTSDP

SEQ ID NO: 57 - protein, 5 aa, synthetic construct
DIIRA

SEQ ID NO: 58 - protein, 12 aa, synthetic construct
SYENVASRRPEG

SEQ ID NO: 59 - protein, 12 aa, synthetic construct
PENSVRRYGLEE

SEQ ID NO: 60 - protein, 12 aa, synthetic construct
LSLASNRPTATS

SEQ ID NO: 61 - protein, 19 aa, synthetic construct
NDVWNRDNSSKRGGTTEAS

SEQ ID NO: 62 - protein, 19 aa, synthetic construct
NRTYSSTSNSTSRSEWDNS

SEQ ID NO: 63 - protein, 7 aa, synthetic construct
ESGHGYF

SEQ ID NO: 64 - protein, 7 aa, synthetic construct
GQHPRPG

SEQ ID NO: 65 - protein, 7 aa, synthetic construct
PSVSPRP

SEQ ID NO: 66 - protein, 7 aa, synthetic construct
VNSTRLP

SEQ ID NO: 67 - protein, 7 aa, synthetic construct
LSPVRPG

SEQ ID NO: 68 - protein, 12 aa, synthetic construct
MSSDPRRPPRDG

SEQ ID NO: 69 - protein, 12 aa, synthetic construct
GARPSEVTTRPG

SEQ ID NO: 70 - protein, 12 aa, synthetic construct
GNEVLGTKPRAP

SEQ ID NO: 71 - protein, 19 aa, synthetic construct
KMRPGAMGTTGEGTRVTRE

SEQ ID NO: 72 - protein, 7 aa, synthetic construct
MNVRGDL

SEQ ID NO: 73 - protein, 7 aa, synthetic construct
ENVRGDL

SEQ ID NO: 74 - protein, 7 aa, synthetic construct
KTLLPTP

SEQ ID NO: 75 - protein, 12 aa, synthetic construct
HLNILSTLWKYR

SEQ ID NO: 76 - protein, 7 aa, synthetic construct
SKAGRSP

SEQ ID NO: 77 - skipped

SEQ ID NO: 78 - protein, 9 aa, synthetic construct
PERTAMSLP

SEQ ID NO: 79 - protein, 7 aa, synthetic construct
ESGLSOS

SEQ ID NO: 80 - protein, 7 aa, synthetic construct
SEGLKNL

SEQ ID NO: 81 - protein, 7 aa, synthetic construct
SLRSPPS

SEQ ID NO: 82 - protein, 7 aa, synthetic construct
RGDLRVS

SEQ ID NO: 83 - protein, 7 aa, synthetic construct
TLAVPFK

SEQ ID NO: 84 - protein, 7 aa, synthetic construct
YTLSQGW

SEQ ID NO: 85 - protein, 671 aa, synthetic construct
MPPIKRQPRGWVLPGYRYLGPFNPLDNGEPVNNADRAAQLHDHAYSELIKSGKNPYLYFNKADEKFIDDLKDDWSIGGIIGSSFFKIKRAVAPALGNKERAQKRHFYFANSNKGAKKTKKSEPKPGTSKMSDTDIQDQQPDTVDAPQNASGGGTGSIGGGKGSGVGISTGGWVGGSHFSDKYVVTKNTRQFITTIQNGHLYKTEAIETTNQSGKSQRCVTTPWTYFNFNQYSCHFSPQDWQRLTNEYKRFRPKAMQVKIYNLQIKQILSNGADTTYNNDLTAGVHIFCDGEHAYPNASHPWDEDVMPDLPYKTWKLFQYGYIPIENELADLDGNAAGGNATEKALLYQMPFFLLENSDHQVLRTGESTEFTFNFDCEWVNNERAYIPPGLMFNPKVPTRRVQYIRQNGSTAASTGRIQPYSKPTSWMTGPGLLSAQRVGPQSSDTAPFMVCTNPEGTHINTGAAGFGSGFDPPSGCLAPTNLEYKLQWYQTPEGTGNNGNIIANPSLSMLRDQLLYKGNQTTYNLVGDIWMFPNQVWDRFPITRENPIWCKKPRADKHTIMDPFDGSIAMDHPPGTIFIKMAKIPVPTATNADSYLNIYCTGQVSCEIVWEVERYATKNWRPERRHTALGMSLGGESNYTPTYHVDPTGAYIQPTSYDQCMPVKTNINKVL

SEQ ID NO: 86 - DNA, 2256 bp, unidentified
  source 1..2256: mol_type = other DNA; note = Canine parvovirus
atggcacctccggcaaagagagccaggagaggtaagggtgtgttagtaaagtggggggaggggaaagatttaataacttaactaagtatgtgtttttttataggacttgtgcctccaggttataaatatcttgggcctgggaacagtcttgaccaaggagaaccaactaacccttctgacgccgctgcaaaagaacacgacgaagcttacgctgcttatcttcgctctggtaaaaacccatacttatatttctcgccagcagatcaacgctttatagatcaaactaaggacgctaaagattggggggggaaaataggacattatttttttagagctaaaaaggcaattgctccagtattaactgatacaccagatcatccatcaacatcaagaccaacaaaaccaactaaaagaagtaaaccaccacctcatattttcatcaatcttgcaaaaaaaaaaaaagccggtgcaggacaagtaaaaagagacaatcttgcaccaatgagtgatggagcagttcaaccagacggtggtcagcctgctgtcagaaatgaaagagctacaggatctgggaacgggtctggaggcgggggtggtggtggttctgggggtgtggggatttctacgggtactttcaataatcagacggaatttaaatttttggaaaacggatgggtggaaatcacagcaaactcaagcagacttgtacatttaaatatgccagaaagtgaaaattatagaagagtggttgtaaataatttggataaaactgcagttaacggaaacatggctttagatgatactcatgcacaaattgtaacaccttggtcattggttgatgcaaatgcttggggagtttggtttaatccaggagattggcaactaattgttaatactatgagtgagttgcatttagttagttttgaacaagaaatttttaatgttgttttaaagactgtttcagagtctgctactcagccaccaacaaaagtttataataatgatttaactgcatcattgatggttgcattagatagtaataatactatgccatttactccagcagctatgagatctgagacattgggtttttatccatggaaaccaaccataccaactccatggagatattattttcaatgggatagaacattaataccatctcatactggaactagtggcacaccaacaaatatataccatggtacagatccagatgacgttcaattttatactattgaaaattctgtgccagtacacttactaagaacaggagatgaatttgctacaggaacatttttttttgattgtaaaccatgtagactaacacatacatggcaaacaaatagagcattgggcttaccaccatttctaaattctttgcctcaagctgaaggaggtactaactttggttatataggagttcaacaagataaaagacgtggtgtaactcaaatgggaaatacaaactatattactgaagctactattatgagaccagctgaggttggttatagtgcaccatattattcttttgaggcgtctacacaagggccatttaaaacacctattgcagcaggacgggggggagcgcaaacagatgaaaatcaagcagcagatggtgatccaagatatgcatttggtagacaacatggtcaaaaaactaccacaacaggagaaacacctgagagatttacatatatagcacatcaagatacaggaagatatccagaaggagattggattcaaaatattaactttaaccttcctgtaacaaatgataatgtattgctaccaacagatccaattggaggtaaagcaggaattaactataccaatatatttaatacttatggtcctttaactgcattaaataatgtaccaccagtttatccaaatggtcaaatttgggataaagaatttgatactgatttaaaaccaagacttcatgtaaatgcaccatttgtttgtcaaaataattgtcctggtcaattatttgtaaaagttgcgcctaatttaacaaatgaatatgatcctgatgcatctgctaatatgtcaagaattgtaacttactcagatttttggtggaaaggtaaattagtatttaaagctaaactaagagcctctcatacttggaatccaattcaacaaatgagtattaatgtagataaccaatttaactatgtaccaagtaatattggaggtatgaaaattgtatatgaaaaatctcaactagcacctagaaaattatattaa

SEQ ID NO: 87 - DNA, 1764 bp, unidentified
  source 1..1764: mol_type = other DNA; note = Minute virus of mice
atgagtgatggcaccagccaacctgacagcggaaacgctgtccactcagctgcaagagttgaacgagcagctgacggccctggaggctctgggggtgggggctctggcgggggtggggttggtgtttctactgggtcttatgataatcaaacgcattatagattcttgggtgacggctgggtagaaattactgcactagcaactagactagtacatttaaacatgcctaaatcagaaaactattgcagaatcagagttcacaatacaacagacacatcagtcaaaggcaacatggcaaaagatgatgctcatgagcaaatttggacaccatggagcttggtggatgctaatgcttggggagtttggctccagccaagtgactggcaatacatttgcaacaccatgagccagcttaacttggtatcacttgatcaagaaatattcaatgtagtgctgaaaactgttacagagcaagacttaggaggtcaagctataaaaatatacaacaatgaccttacagcttgcatgatggttgcagtagactcaaacaacattttgccatacacacctgcagcaaactcaatggaaacacttggtttctacccctggaaaccaaccatagcatcaccatacaggtactatttttgcgttgacagagatctttcagtgacctacgaaaatcaagaaggcacagttgaacataatgtgatgggaacaccaaaaggaatgaattctcaattttttaccattgagaacacacaacaaatcacattgctcagaacaggggacgaatttgccacaggtacttactactttgacacaaattcagttaaactcacacacacgtggcaaaccaaccgtcaacttggacagcctccactgctgtcaacctttcctgaagctgacactgatgcaggtacacttactgctcaagggagcagacatggaacaacacaaatgggggttaactgggtgagtgaagcaatcagaaccagacctgctcaagtaggattttgtcaaccacacaatgactttgaagccagcagagctggaccatttgctgccccaaaagttccagcagatattactcaaggagtagacaaagaagccaatggcagtgttagatacagttatggcaaacagcatggtgaaaattgggcttcacatggaccagcaccagagcgctacacatgggatgaaacaagctttggttcaggtagagacaccaaagatggttttattcaatcagcaccactagttgttccaccaccactaaatggcattcttacaaatgcaaaccctattgggactaaaaatgacattcatttttcaaatgtttttaacagctatggtccactaactgcattttcacacccaagtcctgtataccctcaaggacaaatatgggacaaagaactagatcttgaacacaaacctagacttcacataactgctccatttgtttgtaaaaacaatgcacctggacaaatgttggttagattaggaccaaacctaactgaccaatatgatccaaacggagccacactttctagaattgttacatacggtacatttttctggaaaggaaaactaaccatgagagcaaaacttagagctaacaccacttggaacccagtgtaccaagtaagtgctgaagacaatggcaactcatacatgagtgtaactaaatggttaccaactgctactggaaacatgcagtctgtgccgcttataacaagacctgttgctagaaatacttactaa

SEQ ID NO: 88 - protein, 707 aa, unidentified
  source 1..707: note = Bufavirus-1
MPAIRKARGWVPPGYNYLGPFNQDFSKKPTNPSDNAARKHDLEYNKLIKQGHNPYWNYNHADEDFIKETDQATDWGGKFGNFVFRAKRALAPELAPPAKKKTKTKHTEPEYSHKHIKAGTKRGKPFYLFVNLARKKARMTDTQDVSEQQSDQPSVASTSAKAGGGGGGGGSGVGHSTGNYNNRTEFYYHGDEVTIVCHSSRHIHLNMSESEEYKIYDTDRGPTFPTDQTLQGRDTINDSYHAQVETPWFLINPNSWGTWMNPADFQQLTTTCREVTLEHLDQTLDNIVIKTVSKQGSGAEETTQYNNDLTALLQVALDKSNQLPWVADNMYLDSLGYIPWRPCKLKQYSYHVNFWNTIDIISGPQQNQWQQVKKEIKWDDLQFTPIETTTEIDLLRTGDSWTSGPYKFNTKPTQLSYHWQSTRHTGSVHPTEPPNAIGQQGRNIIDINGWQWGDRSNPMSAATRVSNFHIGYSWPEWRIHYGSGGPAINPGAPFSQAPWSTDPQVRLTQGASEKAIFDYNHGDDDPAHRDQWWQNNLPMTGQTDWAPKNAHQTNVSNNIPSRQEFWTQDYHNTFGPFTAVDDVGIQYPWGAIWTKTPDTTHKPMMSAHAPFICKDGPPGQLLVKLAPNYTENLQTDGLGNNRIVTYATFWWTGKLVLKGKLRLPRQFNLYNLPGRPRGTEAKKFLPNEIGHFELPFMPGRCMPNYTI

SEQ ID NO: 89 - protein, 751 aa, synthetic construct
  VARIANT 27: note = X can be any amino acid
MAPPAKRARRGKGVLVKWGEGKDLITXLSMCFFIGLVPPGYKYLGPGNSLDQGEPTNPSDAAAKEHDEAYAAYLRSGKNPYLYFSPADQRFIDQTKDAKDWGGKIGHYFFRAKKAIAPVLTDTPDHPSTSRPTKPTKRSKPPPHIFINLAKKKKAGAGQVKRDNLAPMSDGAVQPDGGQPAVRNERATGSGNGSGGGGGGGSGGVGISTGTFNNQTEFKFLENGWVEITANSSRLVHLNMPESENYRRVVVNNMDKTAVNGNMALDDIHAQIVTPWSLVDANAWGVWFNPGDWQLIVNTMSELHLVSFEQEIFNVVLKTVSESATQPPTKVYNNDLTASLMVALDSNNTMPFTPAAMRSETLGFYPWKPTIPTPWRYYFQWDRTLIPSHTGTSGTPTNIYHGTDPDDVQFYTIENSVPVHLLRTGDEFATGTFFFDCKPCRLTHTWQTNRALGLPPFLNSLPQSEGATNFGDIGVQQDKRRGVTQMGNTNYITEATIMRPAEVGYSAPYYSFEASTQGPFKTPIAAGRGGAQTYENQAADGDPRYAFGRQHGQKTTTTGETPERFTYIAHQDTGRYPEGDWIQNINFNLPVTNDNVLLPTDPIGGKTGINYTNIFNTYGPLTALNNVPPVYPNGQIWDKEFDTDLKPRLHVNAPFVCQNNCPGQLFVKVAPNLTNEYDPDASANMSRIVTYSDFWWKGKLVFKAKLRASHTWNPIQQMSINVDNQFNYVPSNIGGMKIVYEKSQLAPRKLY

SEQ ID NO: 90 - protein, 727 aa, unidentified
  source 1..727: note = Canine parvovirus
MAPPAKRARRGLVPPGYKYLGPGNSLDQGEPTNPSDAAAKEHDEAYAAYLRSGKNPYLYFSPADQRFIDQTKDAKDWGGKIGHYFFRAKKAIAPVLTDTPDHPSTSRPTKPTKRSKPPPHIFINLAKKKKAGAGQVKRDNLAPMSDGGVQPDGGQPAVRNERATGSGNGSGGGGGGGSGGVGISTGTFNNQTEFKFLENGWVEITANSSRLVHLNMPESENYRRVVVNNLDKTAVNGNMALDDTHAQIVTPWSLVDANAWGVWFNPGDWQLIVNTMSELHLVSFEQEIFNVVLKTVSESATQPPTKVYNNDLTASLMVALDSNNTMPFTPAAMRSETLGFYPWKPTIPTPWRYYFQWDRTLIPSHTGTSGTPTNIYHGTDPDDVQFYTIENSVPVHLLRTGDEFATGTFYFDCKPCRLTHTWQTNRALGLPPFLNSLPQAEGGTNFGYIGVQQDKRRGVTQMGNTNIITEATIMRPAEVGYSAPYYSFEASTQGPFKTPIAAGRGGAQTDENRAADGDPRYAFGRQHGQKTTTTGETPERFTYIAHQDTGRYPEGDWIQNINFNLPVTEDNVLLPTDPIGGKTGINYTNIFNTYGPLTALNNVPPVYPNGQIWDKEFDTDLKPRLHVNAPFVCQNNCPGQLFVKVAPNLTNEYDPDASANMSRIVTYSDFWWKGKLVFKAKLRASHTWNPIQQMSINVDNQFNYVPSNIGGMKIVYEKSQLAPRKLY

SEQ ID NO: 91 - protein, 707 aa, unidentified
  source 1..707: note = Cutavirus
MPAIRKARGWVPPGYNFLGPFNQDFNKEPTNPSDNAAKQHDLEYNKLINQGHNPYWYYNKADEDFIKATDQAPDWGGKFGNFIFRAKKHIAPELAPPAKKKSKTKHPEPEFSHKHIKPGTKRGKPFHIFVNLARKRARMSEPAENTNDQPNDSPVEQGAGQIGGGGGGGGSGVGHSTGDYNNRTEFIYHGDEVTIICHSTRLVHINMSDREDYIIYETDRGQLFPTTQDLQGRDTLNDSYHAKVETPWKLLHANSWGCWFSPADFQQMITTCRDIAPIQMHQKIENIVIKTVSKTGTGETETTNYNNDLTALLQIAQDNSNLLPWAADNFYIDSVGYVPWRACKLPTYCYHVDTWNTIDINQADAPNRWREIKKGIQWDNIQFTPLETMINIDLLRTGDAWQSGNYNFHTKPTNLAYHWQSQRHTGSCHPTVAPLVERGQGTNIQSVNCWQWGDRNNPSSASTRVSNMHIGYSFPEWQIHYSTGGPVINPGSAFSQAPWGSTTEGTRLTQGASEKAIYDWAHGDDQPGARETWWQNNQHVTGQTDWAPKNAHTSELNNNVPAATHFWKNSYHNTFSPFTAVDDHGPQYPWGAIWGKYPDTTHKPMMSAHAPFLLHGPPGQLFVKLAPNYTDTLDNGGVTHPRIVTYGTFWWSGKLIFKGKLRTPRQWNTYNLPSLDKRETMKNTVPNEVGHFELPYMPGRCLPNYTL

SEQ ID NO: 92 - protein, 707 aa, unidentified
  source 1..707: note = Cutavirus
MPAIRKARGWVPPGYNFLGPFNQDFNKEPTNPSDNAAKQHDLEYNKLINQGHNPYWYYNKADEDFIKATDQAPDWGGKFGNFIFRAKKHIAPELAPPAKKKSKTKHSEPEFSHKHIKPGTKRGKPFHIFVNLARKRARMSEPANDTNEQPDNSPVEQGAGQIGGGGGGGGSGVGHSTGDYNNRTEFIYHGDEVTIICHSTRLVHINMSDREDYIIYETDRGPLFPTTQDLQGRDTLNDSYHAKVETPWKLLHANSWGCWFSPADFQQMITTCRDIAPIKMHQKIENIVIKTVSKTGTGETETTNYNNDLTALLQIAQDNSNLLPWAADNFYIDSVGYVPWRACKLPTYCYHVDTWNTIDINQADTPNQWREIKKGIQWDNIQFTPLETMINIDLLRTGDAWESGNYNFHTKPTNLAYHWQSQRHTGSCHPTVAPLVERGQGTNIQSVNCWQWGDRNNPSSASTRVSNIHIGYSFPEWQIHYSTGGPVINPGSAFSQAPWGSTTEGTRLTQGASEKAIYDWSHGDDQPGARETWWQNNQHVTGQTDWAPKNAHTSELNNNVPAATHFWKNSYHNTFSPFTAVDDHGPQYPWGAIWGKYPDTTHKPMMSAHAPFLLHGPPGQLFVKLAPNYTDTLDNGGVTHPRIVTYGTFWWSGQLIFKGKLRTPRQWNTYNLPSLDKRETMKNTVPNEVGHFELPYMPGRCLPNYTL

SEQ ID NO: 93 - protein, 727 aa, unidentified
  source 1..727: note = Feline panleukopenia virus
MAPPAKRARRGLVPPGYKYLGPGNSLDQGEPTNPSDAAAKEHDEAYAAYLRSGKNPYLYFSPADQRFIDQTKDAKDWGGKIGHYFFRAKKAIAPVLTDTPDHPSTSRPTKPTKRSKPPPHIFINLAKKKKAGAGQVKRDNLAPMSDGAVQPDGGQPAVRNERATGSGNGSGGGGGGGSGGVGISTGTFNNQTEFKFLENGWVEITANSSRLVHLNMPESENYKRVVVNNMDKTAVKGNMALDDIHVQIVTPWSLVDANAWGVWFNPGDWQLIVNTMSELHLVSFEQEIFNVVLKTVSESATQPPTKVYNNDLTASLMVALDSNNTMPFTPAAMRSETLGFYPWKPTIPTPWRYYFQWDRTLIPSHTGTSGTPTNVYHGTDPDDVQFYTIENSVPVHLLRTGDEFATGTFFFDCKPCRLTHTWQTNRALGLPPFLNSLPQSEGATNYGDIGVQQDKRRGVTQMGNTDYITEATIMRPAEVGYSAPYYSFEASTQGPFKTPIAAGRGGAQTDENQAADGDPRYAFGRQHGQKTTTTGETPERFTYIAHQDTGRYPEGDWIQNINFNLPVTNDNVLLPTDPIGGKTGINYTNIFNTYGPLTALNNVPPVYPNGQIWDKEFDTDLKPRLHVNAPFVCQNNCPGQLFVKVAPNLTNEYDPDASANMSRIVTYSDFWWKGKLVFKAKLRASHTWNPIQQMSINVDNQFNYVPNNIGAMKIVYEKSQLAPRKLY

SEQ ID NO: 94 - protein, 727 aa, unidentified
  source 1..727: note = Feline panleukopenia virus
MAPPAKRARRGLVPPGYKYLGPGNSLDQGEPTNPSDAAAKEHDEAYAAYLRSGKNPYLYFSPADQRFIDQTKDAKDWGGKIGHYFFRAKKAIAPVLTDTPDHPSTSRPTKPTKRSKPPPHIFINLAKKKKAGAGQVKRDNLAPMSDGAVQPDGGQPAVRNERATGSGNGSGGGGGGGSGGVGISTGTFNNQTEFKFLENGWVEITANSSRLVHLNMPESENYKRVVVNNMDKTAVKGNMALDDTHVQIVTPWSLVDANAWGVWFNPGDWQLIVNTMSELHLVSFEQEIFNVVLKTVSESATQPPTKVYNNDLTASLMVALDSNNTMPFTPAAMRSETLGFYPWKPTIPTPWRYYFQWDRTLIPSHTGTSGTPTNVYHGTDPDDVQFYTIENSVPVHLLRTGDEFATGTFFFDCKPCRLTHTWQTNRALGLPPFLNSLPQSEGATNFGDIGVQQDKRRGVTQMGNTDYITEATIMRPAEVGYSAPYYSFEASTQGPFKTPIAAGRGGAQTDENQAADGDPRYAFGRQHGQKTTTTGETPERFTYIAHQDTGRYPEGDWIQNINFNLPVTNDNVLLPTDPIGGKTGINYTNIFNTYGPLTALNNVPPVYPNGQIWDKEFDTDLKPRLHVNAPFVCQNNCPGQLFVKVAPNLTNEYDPDASANMSRIVTYSDFWWKGKLVFKAKLRASHTWNPIQQMSINVDNQFNYVPNNIGAMKIVYEKSQLAPRKLY

SEQ ID NO: 95 - protein, 729 aa, unidentified
  source 1..729: note = Minute virus of mice
MAPPAKRAKRGWVPPGYKYLGPGNSLDQGEPTNPSDAAAKEHDEAYDQYIKSGKNPYLYFSAADQRFIDQTKDAKDWGGKVGHYFFRTKRAFAPKLATDSEPGTSGVSRAGKRTRPPAYIFINQARAKKKLTSSAAQQSSQTMSDGTSQPDSGNAVHSAARVERAADGPGGSGGGGSGGGGVGVSTGSYDNQTHYRFLGDGWVEITALATRLVHLNMPKSENYCRIRVHNTTDTSVKGNMAKDDAHEQIWTPWSLVDANAWGVWLQPSDWQYICNTMSQLNLVSLDQEIFNVVLKTVTEQDLGGQAIKIYNNDLTACMMVAVDSNNILPYTPAANSMETLGFYPWKPTIASPYRYYFCVDRDLSVTYENQEGTVEHNVMGTPKGMNSQFFTIENTQQITLLRTGDEFATGTYYFDTNSVKLTHTWQTNRQLGQPPLLSTFPEADTDAGTLTAQGSRHGTTQMGVNWVSEAIRTRPAQVGFCQPHNDFEASRAGPFAAPKVPADITQGVDKEANGSVRYSYGKQHGENWASHGPAPERYTWDETSFGSGRDTKDGFIQSAPLVVPPPLNGILTNANPIGTKNDIHFSNVFNSYGPLTAFSHPSPVYPQGQIWDKELDLEHKPRLHITAPFVCKNNAPGQMLVRLGPNLTDQYDPNGATLSRIVTYGTFFWKGKLTMRAKLRANTTWNPVYQVSAEDNGNSYMSVTKWLPTATGNMQSVPLITRPVARNTY

SEQ ID NO: 96 - protein, 715 aa, unidentified
  source 1..715: note = Tusavirus 1
MAPAARPRKGWVPPGYNYLGPGNDLDAGEPTNKSDAAARKHDFAYSAYLKQGLDPYWNFNKADEKFIRDTEGATDWGGRLGHWIFRAKKHILPHLKEPTLAGRKRPAPAHIFVNLANKRKKGLPTRKDQQKDTLDSNAQQPVREADQPDGMAASSSDSGPSSSGGGARAGGVGVSTGDFDNTTLWDFHEDGTATITCNSTRLVHLTRPDSLDYKIIPTQNNTAVQTVGHMMDDDNHTQVLTPWSLVDCNAWGVWLSPHDWQHIMNIGEELELLSLEQEVFNVTLKTATETGPPESRITMYNNDLTAVMMITTDTNNQLPYTPAAIRSETLGFYPWRPTVVPRWRYYFDWDRFLSVTSSSDQSTSIINHSSTQSAIGQFFVIETQLPIALLRTGDSYATGGYKFDCNKVNLGRHWQTTRSLGLPPKIEPPTSESALGTINQNARLGWRWGINDVHETNVVRPCTAGYNHPEWFYTHTLEGPAIDPAPPTSIPSNWGGGTPPDTRASSHNQQRITYNYNHGNKDENLNNFSLNPNIELGSIINQGNFLSYEGNGQQINTTAGVGKNGETATSDPNLVRYMPNTYGVYTAVDHQGPVYPHGQIWDKQIHTDKKPELHCLAPFTCKNNPPGQMFVRIAPNLTDTFNATPTFSEIITYADFWWKGTLKMKIKLRPPHQWNIATVLGAAVNIGDAARFVPNRLGQLEFPVINGRIVPSTVY

SEQ ID NO: 97 - DNA, 2169 bp, synthetic construct
  source 1..2169: mol_type = other DNA
ctggcacctccggcaaagagagccaggagaggatataaatatcttgggcctgggaacagtcttgaccaaggagaaccaactaacccttctgacgccgctgcaaaagaacacgacgaagcttacgctgcttatcttcgctctggtaaaaacccatacttatatttctcgccagcagatcaacgctttatagatcaaactaaggacgctaaagattggggggggaaaataggacattatttttttagagctaaaaaggcaattgctccagtattaactgatacaccagatcatccatcaacatcaagaccaacaaaaccaactaaaagaagtaaaccaccacctcatattttcatcaatcttgcaaaaaaaaaaaaagccggtgcaggacaagtaaaaagagacaatcttgcaccaatgagtgatggagcagttcaaccagacggtggtcaacctgctgtcagaaatgaaagagctacaggatctgggaacgggtctggaggcgggggtggtggtggttctgggggtgtggggatttctacgggtactttcaataatcagacggaatttaaatttttggaaaacggatgggtggaaatcacagcaaactcaagcagacttgtacatttaaatatgccagaaagtgaaaattatagaagagtggttgtaaataatatggataaaactgcagttaacggaaacatggctttagatgatattcatgcacaaattgtaacaccttggtcattggttgatgcaaatgcttggggagtttggtttaatccaggagattggcaactaattgttaatactatgagtgagttgcatttagttagttttgaacaagaaatttttaatgttgttttaaagactgtttcagaatctgctactcagccaccaactaaagtttataataatgatttaactgcatcattgatggttgcattagatagtaataatactatgccatttactccagcagctatgagatctgagacattgggtttttatccatggaaaccaaccataccaactccatggagatattattttcaatgggatagaacattaataccatctcatactggaactagtggcacaccaacaaatatataccatggtacagatccagatgatgttcaattttatactattgaaaattctgtgccagtacacttactaagaacaggtgatgaatttgctacaggaacatttttttttgattgtaaaccatgtagactaacacatacatggcaaacaaatagagcattgggcttaccaccatttctaaattctttgcctcaatctgaaggagctactaactttggtgatataggagttcaacaagataaaagacgtggtgtaactcaaatgggaaatacaaactatattactgaagctactattatgagaccagctgaggttggttatagtgcaccatattattcttttgaggcgtctacacaagggccatttaaaacacctattgcagcaggacgggggggagcgcaaacatatgaaaatcaagcagcagatggtgatccaagatatgcatttggtagacaacatggtcaaaaaactaccacaacaggagaaacacctgagagatttacatatatagcacatcaagatacaggaagatatccagaaggagattggattcaaaatattaactttaaccttcctgtaacgaatgataatgtattgctaccaacagatccaattggaggtaaaacaggaattaactatactaatatatttaatacttatggtcctttaactgcattaaataatgtaccaccagtttatccaaatggtcaaatttgggataaagaatttgatactgacttaaaaccaagacttcatgtaaatgcaccatttgtttgtcaaaataattgtcctggtcaattatttgtaaaagttgcgcctaatttaacaaatgaatatgatcctgatgcatctgctaatatgtcaagaattgtaacttactcagatttttggtggaaaggtaaattagtatttaaagctaaactaagagcctctcatacttggaatccaattcaacaaatgagtattaatgtagataaccaatttaactatgtaccaagtaatattggaggtatgaaaattgtatatgaaaaatctcaactagcacctagaaaattatattaa

SEQ ID NO: 98 - DNA, 402 bp, synthetic construct
  source 1..402: mol_type = other DNA
ctggctccagctattagaaaagccagaggttacaacttcctaggacccttcaatcaagacttcaacaaagaaccaactaatccatcagacaacgctgcaaaacaacacgatttggaatacaacaaactaatcaaccaaggacacaatccttattggtactacaacaaagctgacgaagacttcatcaaagcaacagatcaagcaccagactggggaggaaaatttggcaacttcatcttcagagccaaaaaacacatcgctccagaactggcaccaccagcaaaaaagaaaagcaaaaccaaacacagtgaaccagaattcagccacaaacacatcaaaccaggcaccaaaagaggtaagccttttcatatttttgtaaaccttgctagaaaaagagcccgc

SEQ ID NO: 99 - DNA, 2094 bp, synthetic construct
  source 1..2094: mol_type = other DNA
acgccagctattagaaaagccagaggacccttcaatcaagacttcaacaaagaaccaactaatccatcagacaacgctgcaaaacaacacgatttggaatacaacaaactaatcaaccaaggacacaatccttattggtactacaacaaagctgacgaagacttcatcaaagcaacagatcaagcaccagactggggaggaaaatttggcaacttcatcttcagagccaaaaaacacatcgctccagaactggcaccaccagcaaaaaagaaaagcaaaaccaaacacagtgaaccagaattcagccacaaacacatcaaaccaggcaccaaaagaggtaagccttttcatatttttgtaaaccttgctagaaaaagagcccgcatgtcagaaccagctaatgatacaaatgaacaaccagacaactcccctgttgaacagggtgctggtcaaattggaggaggtggaggtggaggtggaagcggtgtcgggcacagcactggtgattataataataggactgagtttatttatcatggtgatgaagtcacaattatttgccactctacaagactggttcacatcaatatgtcagacagggaagactacatcatctatgaaacagacagaggaccactctttcctaccactcaggacctgcagggtagagacactctaaatgactcttaccatgccaaagtagaaacaccatggaaactactccatgcaaacagctggggctgctggttttcaccagcagacttccaacaaatgatcaccacatgcagagacatagcaccaataaaaatgcaccaaaaaatagaaaacattgtcatcaaaacagtcagtaaaacaggcacaggagaaacagaaacaaccaactacaacaatgacctcacagcactcctacaaattgcacaagacaacagtaacctactaccatgggctgcagataacttttatatagactcagtaggttacgttccatggagagcatgcaaactaccaacctactgctaccacgtagacacttggaatacaattgacataaaccaagcagacacaccaaaccaatggagagaaatcaaaaaaggcatccaatgggacaatatccaattcacaccactagaaactatgataaacattgacttactaagaacaggagatgcctgggaatctggtaactacaatttccacacaaaaccaacaaacctagcttaccattggcaatcacaaagacacacaggcagctgtcacccaacagtagcacctctagttgaaagaggacaaggaaccaacatacaatcagtaaactgttggcaatggggagacagaaacaatccaagctctgcatcaaccagagtatccaatatacatattggatactcatttccagaatggcaaatccactactcaacaggaggaccagtaattaatccaggcagtgcattctcacaagcaccatggggctcaacaactgaaggcaccagactaacccaaggtgcatctgaaaaagccatctatgactggtcccatggagatgaccaaccaggagccagagaaacctggtggcaaaacaaccaacatgtaacaggacaaactgactgggcaccaaaaaatgcacacacctcagaactcaacaacaatgtaccagcagccacacacttctggaaaaacagctatcacaacaccttctcaccattcactgcagtagatgatcatggaccacaatatccatggggagccatctggggaaaatacccagacaccacacacaaaccaatgatgtcagctcacgcaccattcctacttcatggaccacctggacaactctttgtaaaactagcaccaaactatacagacacacttgacaacggaggtgtaacacatcccagaatcgtcacatatggaaccttctggtggtcaggacaactcatctttaaaggaaaactacgcactccaagacaatggaatacctacaacctaccaagcctagacaaaagagaaaccatgaaaaacacagtaccaaatgaagttggtcactttgaactaccatacatgccaggaagatgtctaccaaactacacattgtaa

SEQ ID NO: 100 - DNA, 2169 bp, synthetic construct
  source 1..2169: mol_type = other DNA
ctggcacctccggcaaagagagccaggagaggatataaatatcttgggcctgggaacagtcttgaccaaggagaaccaactaacccttctgacgccgctgcaaaagaacacgacgaagcttacgctgcttatcttcgctctggtaaaaacccatacttatatttctcgccagcagatcaacgctttatagatcaaactaaggacgctaaagattggggggggaaaataggacattatttttttagagctaaaaaggcaattgctccagtattaactgatacaccagatcatccatcaacatcaagaccaacaaaaccaactaaaagaagtaaaccaccacctcatattttcatcaatcttgcaaaaaaaaaaaaagccggtgcaggacaagtaaaaagagacaatcttgcaccaatgagtgatggagcagttcaaccagacggtggtcaacctgctgtcagaaatgaaagagctacaggatctgggaacgggtctggaggcgggggtggtggtggttctgggggtgtggggatttctacgggtactttcaataatcagacggaatttaaatttttggaaaacggatgggtggaaatcacagcaaactcaagcagacttgtacatttaaatatgccagaaagtgaaaattataaaagagtagttgtaaataatatggataaaactgcagttaaaggaaacatggctttagatgatattcatgtacaaattgtaacaccttggtcattggttgatgcaaatgcttggggagtttggtttaatccaggagattggcaactaattgttaatactatgagtgagttgcatttagttagttttgaacaagaaatttttaatgttgttttaaagactgtttcagaatctgctactcagccaccaactaaagtttataataatgatttaactgcatcattgatggttgcattagatagtaataatactatgccatttactccagcagctatgagatctgagacattgggtttttatccatggaaaccaaccataccaactccatggagatattattttcaatgggatagaacattaataccatctcatactggaactagtggcacaccaacaaatatataccatggtacagatccagatgatgttcaattttatactattgaaaattctgtgccagtacacttactaagaacaggtgatgaatttgctacaggaacatttttttttgattgtaaaccatgtagactaacacatacatggcaaacaaatagagcattgggcttaccaccatttttaaattctttgcctcaatctgaaggagctactaactttggtgatataggagttcaacaagataaaagacgtggtgtaactcaaatgggaaatacaaactatattactgaagctactattatgagaccagctgaggttggttatagtgcaccatattattcttttgaggcgtctacacaagggccatttaaaacacctattgcagcaggacgggggggagcgcaaacagatgaaaatcaagcagcagatggtgatccaagatatgcatttggtagacaacatggtcaaaaaactaccacaacaggagaaacacctgagagatttacatatatagcacatcaagatacaggaagatatccagaaggagattggattcaaaatattaactttaaccttcctgtaacaaatgataatgtattgctaccaacagatccaattggaggtaaaacaggaattaactatactaatatatttaatacttatggtcctttaactgcattaaataatgtaccaccagtttatccaaatggtcaaatttgggataaagaatttgatactgacttaaaaccaagacttcatgtaaatgcaccatttgtttgtcaaaataattgtcctggtcaattatttgtaaaagttgcgcctaatttaacaaatgaatatgatcctgatgcatctgctaatatgtcaagaattgtaacttactcagatttttggtggaaaggtaaattagtatttaaagctaaactaagagcctctcatacttggaatccaattcaacaaatgagtattaatgtagataaccaatttaactatgtaccaagtaatattggagctatgaaaattgtatatgaaaaatctcaactagcacctagaaaattatattaa

SEQ ID NO: 101 - DNA, 2175 bp, synthetic construct
  source 1..2175: mol_type = other DNA
acggcgcctccagctaaaagagctaaaagaggctacaagtacctgggaccagggaacagccttgaccaaggagaaccaaccaatccatctgacgccgctgccaaagagcacgacgaggcctacgatcaatacatcaaatctggaaaaaatccttacctgtacttctctgctgctgatcaacgctttattgaccaaaccaaggacgccaaagactggggaggcaaggttggtcactacttttttagaaccaagcgcgcttttgcacctaagcttgctactgactctgaacctggaacttctggtgtaagcagagctggtaaacgcactagaccacctgcttacatttttattaaccaagccagagctaaaaaaaaacttacttcttctgctgcacagcaaagcagtcaaaccatgagtgatggcaccagccaacctgacagcggaaacgctgtccactcagctgcaagagttgaacgagcagctgacggccctggaggctctgggggtgggggctctggcgggggtggggttggtgtttctactgggtcttatgataatcaaacgcattatagattcttgggtgacggctgggtagaaattactgcactagcaactagactagtacatttaaacatgcctaaatcagaaaactattgcagaatcagagttcacaatacaacagacacatcagtcaaaggcaacatggcaaaagatgatgctcatgagcaaatttggacaccatggagcttggtggatgctaatgcttggggagtttggctccagccaagtgactggcaatacatttgcaacaccatgagccagcttaacttggtatcacttgatcaagaaatattcaatgtagtgctgaaaactgttacagagcaagacttaggaggtcaagctataaaaatatacaacaatgaccttacagcttgcatgatggttgcagtagactcaaacaacattttgccatacacacctgcagcaaactcaatggaaacacttggtttctacccctggaaaccaaccatagcatcaccatacaggtactatttttgcgttgacagagatctttcagtgacctacgaaaatcaagaaggcacagttgaacataatgtgatgggaacaccaaaaggaatgaattctcaattttttaccattgagaacacacaacaaatcacattgctcagaacaggggacgaatttgccacaggtacttactactttgacacaaattcagttaaactcacacacacgtggcaaaccaaccgtcaacttggacagcctccactgctgtcaacctttcctgaagctgacactgatgcaggtacacttactgctcaagggagcagacatggaacaacacaaatgggggttaactgggtgagtgaagcaatcagaaccagacctgctcaagtaggattttgtcaaccacacaatgactttgaagccagcagagctggaccatttgctgccccaaaagttccagcagatattactcaaggagtagacaaagaagccaatggcagtgttagatacagttatggcaaacagcatggtgaaaattgggcttcacatggaccagcaccagagcgctacacatgggatgaaacaagctttggttcaggtagagacaccaaagatggttttattcaatcagcaccactagttgttccaccaccactaaatggcattcttacaaatgcaaaccctattgggactaaaaatgacattcatttttcaaatgtttttaacagctatggtccactaactgcattttcacacccaagtcctgtataccctcaaggacaaatatgggacaaagaactagatcttgaacacaaacctagacttcacataactgctccatttgtttgtaaaaacaatgcacctggacaaatgttggttagattaggaccaaacctaactgaccaatatgatccaaacggagccacactttctagaattgttacatacggtacatttttctggaaaggaaaactaaccatgagagcaaaacttagagctaacaccacttggaacccagtgtaccaagtaagtgctgaagacaatggcaactcatacatgagtgtaactaaatggttaccaactgctactggaaacatgcagtctgtgccgcttataacaagacctgttgctagaaatacttactaa

SEQ ID NO: 102 - DNA, 2193 bp, synthetic construct
  source 1..2193: mol_type = other DNA
acggcacctccagctaaaagagctaaaagaggctacaagtacctgggaccagggaacagccttgaccaaggagaaccaaccaacccttctgacgccgctgccaaagaacacgacgaagcctacgaccaatacatcaaatctggaaaaaatccttacctgtacttctctcctgctgatcaacgcttcattgaccaaaccaaagacgccaaggactggggcggcaaggttggtcactacttttttagaaccaagcgagcttttgcacctaagctttctactgactctgaacctggcacttctggtgtgagcagacctggtaaacgaactaaaccacctgctcacatttttgtaaatcaagccagagctaaaaaaaaacgcgcttctcttgctgcacagcagaggactctgacaatgagtgatggcaccgaaacaaaccaaccagacactggaatcgctaatgctagagttgagcgatcagctgacggaggtggaagctctgggggtgggggctctggcgggggtgggattggtgtttctactgggacttatgataatcaaacgacttataagtttttgggagatggatgggtagaaataactgcacatgcttctagacttttgcacttgggaatgcctccttcagaaaactactgccgcgtcaccgttcacaataatcaaacaacaggacacggaactaaggtaaagggaaacatggcctatgatgacacacatcaacaaatttggacaccatggagcttggtagatgctaatgcttggggagtttggttccaaccaagtgactggcagttcattcaaaacagcatggaatcgctgaatcttgactcattgagccaagaactatttaatgtagtagtcaaaacagtcactgaacaacaaggagctggccaagatgccattaaagtctataataatgacttgacggcctgtatgatggttgctctggatagtaacaacatactgccttacacacctgcagctcaaacatcagaaacacttggtttctacccatggaaaccaaccgcaccagctccttacagatactactttttcatgcctagacaactcagtgtaacctctagcaactctgctgaaggaactcaaatcacagacaccattggagagccacaggcactaaactctcaattttttactattgagaacaccttgcctattactctcctgcgcacaggtgatgagtttacaactggcacctacatctttaacactgacccacttaaacttactcacacatggcaaaccaacagacacttgggcatgcctccaagaataactgacctaccaacatcagatacagcaacagcatcactaactgcaaatggagacagatttggatcaacacaaacacagaatgtgaactatgtcacagaggctttgcgcaccaggcctgctcagattggcttcatgcaacctcatgacaactttgaagcaaacagaggtggcccatttaaggttccagtggtaccgctagacataacagctggcgaggaccatgatgcaaacggagccatacgatttaactatggcaaacaacatggcgaagattgggccaaacaaggagcagcaccagaaaggtacacatgggatgcaattgatagtgcagctgggagggacacagctagatgctttgtacaaagtgcaccaatatctattccaccaaaccaaaaccagatcttgcagcgagaagacgccatagctggcagaactaacatgcattatactaatgtttttaacagctatggtccacttagtgcatttcctcatccagatcccatttatccaaatggacaaatttgggacaaagaattggacctggaacacaaacctagactacacgtaactgcaccatttgtttgtaaaaacaacccaccaggtcaactatttgttcgcttggggcctaatctgactgaccaatttgacccaaacagcacaactgtttctcgcattgttacatatagcactttttactggaagggtattttgaaattcaaagccaaactaagaccaaatctgacctggaatcctgtataccaagcaaccacagactctgttgccaattcttacatgaatgttaagaaatggctcccatctgcaactggcaacatgcactctgatccattgatttgtagacctgtgcctcacatgacatactaa

SEQ ID NO: 103 - protein, 702 aa, synthetic construct
MPAIRKARGYNYLGPFNQDFSKKPTNPSDNAARKHDLEYNKLIKQGHNPYWNYNHADEDFIKETDQATDWGGKFGNFVFRAKRALAPELAPPAKKKTKTKHTEPEYSHKHIKAGTKRGKPFYLFVNLARKKARMTDTQDVSEQQSDQPSVASTSAKAGGGGGGGGSGVGHSTGNYNNRTEFYYHGDEVTIVCHSSRHIHLNMSESEEYKIYDTDRGPTFPTDQTLQGRDTINDSYHAQVETPWFLINPNSWGTWMNPADFQQLTTTCREVTLEHLDQTLDNIVIKTVSKQGSGAEETTQYNNDLTALLQVALDKSNQLPWVADNMYLDSLGYIPWRPCKLKQYSYHVNFWNTIDIISGPQQNQWQQVKKEIKWDDLQFTPIETTTEIDLLRTGDSWTSGPYKFNTKPTQLSYHWQSTRHTGSVHPTEPPNAIGQQGRNIIDINGWQWGDRSNPMSAATRVSNFHIGYSWPEWRIHYGSGGPAINPGAPFSQAPWSTDPQVRLTQGASEKAIFDYNHGDDDPAHRDQWWQNNLPMTGQTDWAPKNAHQTNVSNNIPSRQEFWTQDYHNTFGPFTAVDDVGIQYPWGAIWTKTPDTTHKPMMSAHAPFICKDGPPGQLLVKLAPNYTENLQTDGLGNNRIVTYATFWWTGKLVLKGKLRLPRQFNLYNLPGRPRGTEAKKFLPNEIGHFELPFMPGRCMPNYTI

SEQ ID NO: 104 - protein, 722 aa, synthetic construct
LAPPAKRARRGYKYLGPGNSLDQGEPTNPSDAAAKEHDEAYAAYLRSGKNPYLYFSPADQRFIDQTKDAKDWGGKIGHYFFRAKKAIAPVLTDTPDHPSTSRPTKPTKRSKPPPHIFINLAKKKKAGAGQVKRDNLAPMSDGAVQPDGGQPAVRNERATGSGNGSGGGGGGGSGGVGISTGTFNNQTEFKFLENGWVEITANSSRLVHLNMPESENYRRVVVNNMDKTAVNGNMALDDIHAQIVTPWSLVDANAWGVWFNPGDWQLIVNTMSELHLVSFEQEIFNVVLKTVSESATQPPTKVYNNDLTASLMVALDSNNTMPFTPAAMRSETLGFYPWKPTIPTPWRYYFQWDRTLIPSHTGTSGTPTNIYHGTDPDDVQFYTIENSVPVHLLRTGDEFATGTFFFDCKPCRLTHTWQTNRALGLPPFLNSLPQSEGATNFGDIGVQQDKRRGVTQMGNTNYITEATIMRPAEVGYSAPYYSFEASTQGPFKTPIAAGRGGAQTYENQAADGDPRYAFGRQHGQKTTTTGETPERFTYIAHQDTGRYPEGDWIQNINFNLPVTNDNVLLPTDPIGGKTGINYTNIFNTYGPLTALNNVPPVYPNGQIWDKEFDTDLKPRLHVNAPFVCQNNCPGQLFVKVAPNLTNEYDPDASANMSRIVTYSDFWWKGKLVFKAKLRASHTWNPIQQMSINVDNQFNYVPSNIGGMKIVYEKSQLAPRKLY

SEQ ID NO: 105 - protein, 702 aa, synthetic construct
MPAIRKARGYNFLGPFNQDFNKEPTNPSDNAAKQHDLEYNKLINQGHNPYWYYNKADEDFIKATDQAPDWGGKFGNFIFRAKKHIAPELAPPAKKKSKTKHPEPEFSHKHIKPGTKRGKPFHIFVNLARKRARMSEPAENTNDQPNDSPVEQGAGQIGGGGGGGGSGVGHSTGDYNNRTEFIYHGDEVTIICHSTRLVHINMSDREDYIIYETDRGQLFPTTQDLQGRDTLNDSYHAKVETPWKLLHANSWGCWFSPADFQQMITTCRDIAPIQMHQKIENIVIKTVSKTGTGETETTNYNNDLTALLQIAQDNSNLLPWAADNFYIDSVGYVPWRACKLPTYCYHVDTWNTIDINQADAPNRWREIKKGIQWDNIQFTPLETMINIDLLRTGDAWQSGNYNFHTKPTNLAYHWQSQRHTGSCHPTVAPLVERGQGTNIQSVNCWQWGDRNNPSSASTRVSNMHIGYSFPEWQIHYSTGGPVINPGSAFSQAPWGSTTEGTRLTQGASEKAIYDWAHGDDQPGARETWWQNNQHVTGQTDWAPKNAHTSELNNNVPAATHFWKNSYHNTFSPFTAVDDHGPQYPWGAIWGKYPDTTHKPMMSAHAPFLLHGPPGQLFVKLAPNYTDTLDNGGVTHPRIVTYGTFWWSGKLIFKGKLRTPRQWNTYNLPSLDKRETMKNTVPNEVGHFELPYMPGRCLPNYTL

SEQ ID NO: 106 - protein, 697 aa, synthetic construct
TPAIRKARGPFNQDFNKEPTNPSDNAAKQHDLEYNKLINQGHNPYWYYNKADEDFIKATDQAPDWGGKFGNFIFRAKKHIAPELAPPAKKKSKTKHSEPEFSHKHIKPGTKRGKPFHIFVNLARKRARMSEPANDTNEQPDNSPVEQGAGQIGGGGGGGGSGVGHSTGDYNNRTEFIYHGDEVTIICHSTRLVHINMSDREDYIIYETDRGPLFPTTQDLQGRDTLNDSYHAKVETPWKLLHANSWGCWFSPADFQQMITTCRDIAPIKMHQKIENIVIKTVSKTGTGETETTNYNNDLTALLQIAQDNSNLLPWAADNFYIDSVGYVPWRACKLPTYCYHVDTWNTIDINQADTPNQWREIKKGIQWDNIQFTPLETMINIDLLRTGDAWESGNYNFHTKPTNLAYHWQSQRHTGSCHPTVAPLVERGQGTNIQSVNCWQWGDRNNPSSASTRVSNIHIGYSFPEWQIHYSTGGPVINPGSAFSQAPWGSTTEGTRLTQGASEKAIYDWSHGDDQPGARETWWQNNQHVTGQTDWAPKNAHTSELNNNVPAATHFWKNSYHNTFSPFTAVDDHGPQYPWGAIWGKYPDTTHKPMMSAHAPFLLHGPPGQLFVKLAPNYTDTLDNGGVTHPRIVTYGTFWWSGQLIFKGKLRTPRQWNTYNLPSLDKRETMKNTVPNEVGHFELPYMPGRCLPNYTL

SEQ ID NO: 107 - protein, 722 aa, synthetic construct
LAPPAKRARRGYKYLGPGNSLDQGEPTNPSDAAAKEHDEAYAAYLRSGKNPYLYFSPADQRFIDQTKDAKDWGGKIGHYFFRAKKAIAPVLTDTPDHPSTSRPTKPTKRSKPPPHIFINLAKKKKAGAGQVKRDNLAPMSDGAVQPDGGQPAVRNERATGSGNGSGGGGGGGSGGVGISTGTFNNQTEFKFLENGWVEITANSSRLVHLNMPESENYKRVVVNNMDKTAVKGNMALDDIHVQIVTPWSLVDANAWGVWFNPGDWQLIVNTMSELHLVSFEQEIFNVVLKTVSESATQPPTKVYNNDLTASLMVALDSNNTMPFTPAAMRSETLGFYPWKPTIPTPWRYYFQWDRTLIPSHTGTSGTPTNIYHGTDPDDVQFYTIENSVPVHLLRTGDEFATGTFFFDCKPCRLTHTWQTNRALGLPPFLNSLPQSEGATNFGDIGVQQDKRRGVTQMGNTNYITEATIMRPAEVGYSAPYYSFEASTQGPFKTPIAAGRGGAQTDENQAADGDPRYAFGRQHGQKTTTTGETPERFTYIAHQDTGRYPEGDWIQNINFNLPVTNDNVLLPTDPIGGKTGINYTNIFNTYGPLTALNNVPPVYPNGQIWDKEFDTDLKPRLHVNAPFVCQNNCPGQLFVKVAPNLTNEYDPDASANMSRIVTYSDFWWKGKLVFKAKLRASHTWNPIQQMSINVDNQFNYVPSNIGAMKIVYEKSQLAPRKLY

SEQ ID NO: 108 - protein, 724 aa, synthetic construct
TAPPAKRAKRGYKYLGPGNSLDQGEPTNPSDAAAKEHDEAYDQYIKSGKNPYLYFSAADQRFIDQTKDAKDWGGKVGHYFFRTKRAFAPKLATDSEPGTSGVSRAGKRTRPPAYIFINQARAKKKLTSSAAQQSSQTMSDGTSQPDSGNAVHSAARVERAADGPGGSGGGGSGGGGVGVSTGSYDNQTHYRFLGDGWVEITALATRLVHLNMPKSENYCRIRVHNTTDTSVKGNMAKDDAHEQIWTPWSLVDANAWGVWLQPSDWQYICNTMSQLNLVSLDQEIFNVVLKTVTEQDLGGQAIKIYNNDLTACMMVAVDSNNILPYTPAANSMETLGFYPWKPTIASPYRYYFCVDRDLSVTYENQEGTVEHNVMGTPKGMNSQFFTIENTQQITLLRTGDEFATGTYYFDTNSVKLTHTWQTNRQLGQPPLLSTFPEADTDAGTLTAQGSRHGTTQMGVNWVSEAIRTRPAQVGFCQPHNDFEASRAGPFAAPKVPADITQGVDKEANGSVRYSYGKQHGENWASHGPAPERYTWDETSFGSGRDTKDGFIQSAPLVVPPPLNGILTNANPIGTKNDIHFSNVFNSYGPLTAFSHPSPVYPQGQIWDKELDLEHKPRLHITAPFVCKNNAPGQMLVRLGPNLTDQYDPNGATLSRIVTYGTFFWKGKLTMRAKLRANTTWNPVYQVSAEDNGNSYMSVTKWLPTATGNMQSVPLITRPVARNTY

SEQ ID NO: 109 - protein, 710 aa, synthetic construct
MAPAARPRKGYNYLGPGNDLDAGEPTNKSDAAARKHDFAYSAYLKQGLDPYWNFNKADEKFIRDTEGATDWGGRLGHWIFRAKKHILPHLKEPTLAGRKRPAPAHIFVNLANKRKKGLPTRKDQQKDTLDSNAQQPVREADQPDGMAASSSDSGPSSSGGGARAGGVGVSTGDFDNTTLWDFHEDGTATITCNSTRLVHLTRPDSLDYKIIPTQNNTAVQTVGHMMDDDNHTQVLTPWSLVDCNAWGVWLSPHDWQHIMNIGEELELLSLEQEVFNVTLKTATETGPPESRITMYNNDLTAVMMITTDTNNQLPYTPAAIRSETLGFYPWRPTVVPRWRYYFDWDRFLSVTSSSDQSTSIINHSSTQSAIGQFFVIETQLPIALLRTGDSYATGGYKFDCNKVNLGRHWQTTRSLGLPPKIEPPTSESALGTINQNARLGWRWGINDVHETNVVRPCTAGYNHPEWFYTHTLEGPAIDPAPPTSIPSNWGGGTPPDTRASSHNQQRITYNYNHGNKDENLNNFSLNPNIELGSIINQGNFLSYEGNGQQINTTAGVGKNGETATSDPNLVRYMPNTYGVYTAVDHQGPVYPHGQIWDKQIHTDKKPELHCLAPFTCKNNPPGQMFVRIAPNLTDTFNATPTFSEIITYADFWWKGTLKMKIKLRPPHQWNIATVLGAAVNIGDAARFVPNRLGQLEFPVINGRIVPSTVY

SEQ ID NO: 110 - protein, 730 aa, synthetic construct
TAPPAKRAKRGYKYLGPGNSLDQGEPTNPSDAAAKEHDEAYDQYIKSGKNPYLYFSPADQRFIDQTKDAKDWGGKVGHYFFRTKRAFAPKLSTDSEPGTSGVSRPGKRTKPPAHIFVNQARAKKKRASLAAQQRTLTMSDGTETNQPDTGIANARVERSADGGGSSGGGGSGGGGIGVSTGTYDNQTTYKFLGDGWVEITAHASRLLHLGMPPSENYCRVTVHNNQTTGHGTKVKGNMAYDDTHQQIWTPWSLVDANAWGVWFQPSDWQFIQNSMESLNLDSLSQELFNVVVKTVTEQQGAGQDAIKVYNNDLTACMMVALDSNNILPYTPAAQTSETLGFYPWKPTAPAPYRYYFFMPRQLSVTSSNSAEGTQITDTIGEPQALNSQFFTIENTLPITLLRTGDEFTTGTYIFNTDPLKLTHTWQTNRHLGMPPRITDLPTSDTATASLTANGDRFGSTQTQNVNYVTEALRTRPAQIGFMQPHDNFEANRGGPFKVPVVPLDITAGEDHDANGAIRFNYGKQHGEDWAKQGAAPERYTWDAIDSAAGRDTARCFVQSAPISIPPNQNQILQREDAIAGRTNMHYTNVFNSYGPLSAFPHPDPIYPNGQIWDKELDLEHKPRLHVTAPFVCKNNPPGQLFVRLGPNLTDQFDPNSTTVSRIVTYSTFYWKGILKFKAKLRPNLTWNPVYQATTDSVANSYMNVKKWLPSATGNMHSDPLICRPVPHMTY

SEQ ID NO: 111 - protein, 569 aa, synthetic construct
MTDTQDVSEQQSDQPSVASTSAKAGGGGGGGGSGVGHSTGNYNNRTEFYYHGDEVTIVCHSSRHIHLNMSESEEYKIYDTDRGPTFPTDQTLQGRDTINDSYHAQVETPWFLINPNSWGTWMNPADFQQLTTTCREVTLEHLDQTLDNIVIKTVSKQGSGAEETTQYNNDLTALLQVALDKSNQLPWVADNMYLDSLGYIPWRPCKLKQYSYHVNFWNTIDIISGPQQNQWQQVKKEIKWDDLQFTPIETTTEIDLLRTGDSWTSGPYKFNTKPTQLSYHWQSTRHTGSVHPTEPPNAIGQQGRNIIDINGWQWGDRSNPMSAATRVSNFHIGYSWPEWRIHYGSGGPAINPGAPFSQAPWSTDPQVRLTQGASEKAIFDYNHGDDDPAHRDQWWQNNLPMTGQTDWAPKNAHQTNVSNNIPSRQEFWTQDYHNTFGPFTAVDDVGIQYPWGAIWTKTPDTTHKPMMSAHAPFICKDGPPGQLLVKLAPNYTENLQTDGLGNNRIVTYATFWWTGKLVLKGKLRLPRQFNLYNLPGRPRGTEAKKFLPNEIGHFELPFMPGRCMPNYTI

SEQ ID NO: 112 - protein, 584 aa, synthetic construct
MSDGAVQPDGGQPAVRNERATGSGNGSGGGGGGGSGGVGISTGTFNNQTEFKFLENGWVEITANSSRLVHLNMPESENYRRVVVNNLDKTAVNGNMALDDTHAQIVTPWSLVDANAWGVWFNPGDWQLIVNTMSELHLVSFEQEIFNVVLKTVSESATQPPTKVYNNDLTASLMVALDSNNTMPFTPAAMRSETLGFYPWKPTIPTPWRYYFQWDRTLIPSHTGTSGTPTNIYHGTDPDDVQFYTIENSVPVHLLRTGDEFATGTFFFDCKPCRLTHTWQTNRALGLPPFLNSLPQSEGGTNFGYIGVQQDKRRGVTQMGNTNYITEATIMRPAEVGYSAPYYSFEASTQGPFKTPIAAGRGGAQTDENQAADGDPRYAFGRQHGQKTTTTGETPERFTYIAHQDTGRYPEGDWIQNINFNLPVTDDNVLLPTDPIGGKTGINYTNIFNTYGPLTALNNVPPVYPNGQIWDKEFDTDLKPRLHVNAPFVCQNNCPGQLFVKVAPNLTNEYDPDASANMSRIVTYSDFWWKGKLVFKAKLRASHTWNPIQQMSINVDNQFNYVPSNIGGMKIVYEKSQLAPRKLY

SEQ ID NO: 113 - DNA, 1752 bp, synthetic construct
  source 1..1752: mol_type = other DNA
atgagcgacggcgccgtgcagcccgacggcggccagcccgccgtgcgcaacgagcgcgccaccggcagcggcaacggcagcggcggcggcggcggcggcggcagcggcggcgtgggcatcagcaccggcaccttcaacaaccagaccgagttcaagttcctggagaacggctgggtggagatcaccgccaacagcagccgcctggtgcacctgaacatgcccgagagcgagaactaccgccgcgtggtggtgaacaacatggacaagaccgccgtgaacggcaacatggccctggacgacatccacgcccagatcgtgaccccctggagcctggtggacgccaacgcctggggcgtgtggttcaaccccggcgactggcagctgatcgtgaacaccatgagcgagctgcacctggtgagcttcgagcaggagatcttcaacgtggtgctgaagaccgtgagcgagagcgccacccagccccccaccaaggtgtacaacaacgacctgaccgccagcctgatggtggccctggacagcaacaacaccatgcccttcacccccgccgccatgcgcagcgagaccctgggcttctacccctggaagcccaccatccccaccccctggcgctactacttccagtgggaccgcaccctgatccccagccacaccggcaccagcggcacccccaccaacatctaccacggcaccgaccccgacgacgtgcagttctacaccatcgagaacagcgtgcccgtgcacctgctgcgcaccggcgacgagttcgccaccggcaccttcttcttcgactgcaagccctgccgcctgacccacacctggcagaccaaccgcgccctgggcctgccccccttcctgaacagcctgccccagagcgagggcgccaccaacttcggcgacatcggcgtgcagcaggacaagcgccgcggcgtgacccagatgggcaacaccaactacatcaccgaggccaccatcatgcgccccgccgaggtgggctacagcgccccctactacagcttcgaggccagcacccagggccccttcaagacccccatcgccgccggccgcggcggcgcccagacctacgagaaccaggccgccgacggcgacccccgctacgccttcggccgccagcacggccagaagaccaccaccaccggcgagacccccgagcgcttcacctacatcgcccaccaggacaccggccgctaccccgagggcgactggatccagaacatcaacttcaacctgcccgtgaccaacgacaacgtgctgctgcccaccgaccccatcggcggcaagaccggcatcaactacaccaacatcttcaacacctacggccccctgaccgccctgaacaacgtgccccccgtgtaccccaacggccagatctgggacaaggagttcgacaccgacctgaagccccgcctgcacgtgaacgcccccttcgtgtgccagaacaactgccccggccagctgttcgtgaaggtggcccccaacctgaccaacgagtacgaccccgacgccagcgccaacatgagccgcatcgtgacctacagcgacttctggtggaagggcaagctggtgttcaaggccaagctgcgcgccagccacacctggaaccccatccagcagatgagcatcaacgtggacaaccagttcaactacgtgcccagcaacatcggcggcatgaagatcgtgtacgagaagagccagctggccccccgcaagctgtac

SEQ ID NO: 114 - protein, 569 aa, synthetic construct
MSEPANDTNEQPDNSPVEQGAGQIGGGGGGGGSGVGHSTGDYNNRTEFIYHGDEVTIICHSTRLVHINMSDREDYIIYETDRGPLFPTTQDLQGRDTLNDSYHAKVETPWKLLHANSWGCWFSPADFQQMITTCRDIAPIKMHQKIENIVIKTVSKTGTGETETTNYNNDLTALLQIAQDNSNLLPWAADNFYIDSVGYVPWRACKLPTYCYHVDTWNTIDINQADTPNQWREIKKGIQWDNIQFTPLETMINIDLLRTGDAWESGNYNFHTKPTNLAYHWQSQRHTGSCHPTVAPLVERGQGTNIQSVNCWQWGDRNNPSSASTRVSNIHIGYSFPEWQIHYSTGGPVINPGSAFSQAPWGSTTEGTRLTQGASEKAIYDWSHGDDQPGARETWWQNNQHVTGQTDWAPKNAHTSELNNNVPAATHFWKNSYHNTFSPFTAVDDHGPQYPWGAIWGKYPDTTHKPMMSAHAPFLLHGPPGQLFVKLAPNYTDTLDNGGVTHPRIVTYGTFWWSGQLIFKGKLRTPRQWNTYNLPSLDKRETMKNTVPNEVGHFELPYMPGRCLPNYTL

SEQ ID NO: 115 - DNA, 1707 bp, synthetic construct
  source 1..1707: mol_type = other DNA
atgagcgagcccgccaacgacaccaacgagcagcccgacaacagccccgtggagcagggcgccggccagatcggcggcggcggcggcggcggcggcagcggcgtgggccacagcaccggcgactacaacaaccgcaccgagttcatctaccacggcgacgaggtgaccatcatctgccacagcacccgcctggtgcacatcaacatgagcgaccgcgaggactacatcatctacgagaccgaccgcggccccctgttccccaccacccaggacctgcagggccgcgacaccctgaacgacagctaccacgccaaggtggagaccccctggaagctgctgcacgccaacagctggggctgctggttcagccccgccgacttccagcagatgatcaccacctgccgcgacatcgcccccatcaagatgcaccagaagatcgagaacatcgtgatcaagaccgtgagcaagaccggcaccggcgagaccgagaccaccaactacaacaacgacctgaccgccctgctgcagatcgcccaggacaacagcaacctgctgccctgggccgccgacaacttctacatcgacagcgtgggctacgtgccctggcgcgcctgcaagctgcccacctactgctaccacgtggacacctggaacaccatcgacatcaaccaggccgacacccccaaccagtggcgcgagatcaagaagggcatccagtgggacaacatccagttcacccccctggagaccatgatcaacatcgacctgctgcgcaccggcgacgcctgggagagcggcaactacaacttccacaccaagcccaccaacctggcctaccactggcagagccagcgccacaccggcagctgccaccccaccgtggcccccctggtggagcgcggccagggcaccaacatccagagcgtgaactgctggcagtggggcgaccgcaacaaccccagcagcgccagcacccgcgtgagcaacatccacatcggctacagcttccccgagtggcagatccactacagcaccggcggccccgtgatcaaccccggcagcgccttcagccaggccccctggggcagcaccaccgagggcacccgcctgacccagggcgccagcgagaaggccatctacgactggagccacggcgacgaccagcccggcgcccgcgagacctggtggcagaacaaccagcacgtgaccggccagaccgactgggcccccaagaacgcccacaccagcgagctgaacaacaacgtgcccgccgccacccacttctggaagaacagctaccacaacaccttcagccccttcaccgccgtggacgaccacggcccccagtacccctggggcgccatctggggcaagtaccccgacaccacccacaagcccatgatgagcgcccacgcccccttcctgctgcacggcccccccggccagctgttcgtgaagctggcccccaactacaccgacaccctggacaacggcggcgtgacccacccccgcatcgtgacctacggcaccttctggtggagcggccagctgatcttcaagggcaagctgcgcaccccccgccagtggaacacctacaacctgcccagcctggacaagcgcgagaccatgaagaacaccgtgcccaacgaggtgggccacttcgagctgccctacatgcccggccgctgcctgcccaactacaccctg

SEQ ID NO: 116 - protein, 584 aa, synthetic construct
MSDGAVQPDGGQPAVRNERATGSGNGSGGGGGGGSGGVGISTGTFNNQTEFKFLENGWVEITANSSRLVHLNMPESENYKRVVVNNMDKTAVKGNMALDDIHVQIVTPWSLVDANAWGVWFNPGDWQLIVNTMSELHLVSFEQEIFNVVLKTVSESATQPPTKVYNNDLTASLMVALDSNNTMPFTPAAMRSETLGFYPWKPTIPTPWRYYFQWDRTLIPSHTGTSGTPTNIYHGTDPDDVQFYTIENSVPVHLLRTGDEFATGTFFFDCKPCRLTHTWQTNRALGLPPFLNSLPQSEGATNFGDIGVQQDKRRGVTQMGNTNYITEATIMRPAEVGYSAPYYSFEASTQGPFKTPIAAGRGGAQTDENQAADGDPRYAFGRQHGQKTTTTGETPERFTYIAHQDTGRYPEGDWIQNINFNLPVTNDNVLLPTDPIGGKTGINYTNIFNTYGPLTALNNVPPVYPNGQIWDKEFDTDLKPRLHVNAPFVCQNNCPGQLFVKVAPNLTNEYDPDASANMSRIVTYSDFWWKGKLVFKAKLRASHTWNPIQQMSINVDNQFNYVPSNIGAMKIVYEKSQLAPRKLY

SEQ ID NO: 117 - protein, 565 aa, synthetic construct
MAASSSDSGPSSSGGGARAGGVGVSTGDFDNTTLWDFHEDGTATITCNSTRLVHLTRPDSLDYKIIPTQNNTAVQTVGHMMDDDNHTQVLTPWSLVDCNAWGVWLSPHDWQHIMNIGEELELLSLEQEVFNVTLKTATETGPPESRITMYNNDLTAVMMITTDTNNQLPYTPAAIRSETLGFYPWRPTVVPRWRYYFDWDRFLSVTSSSDQSTSIINHSSTQSAIGQFFVIETQLPIALLRTGDSYATGGYKFDCNKVNLGRHWQTTRSLGLPPKIEPPTSESALGTINQNARLGWRWGINDVHETNVVRPCTAGYNHPEWFYTHTLEGPAIDPAPPTSIPSNWGGGTPPDTRASSHNQQRITYNYNHGNKDENLNNFSLNPNIELGSIINQGNFLSYEGNGQQINTTAGVGKNGETATSDPNLVRYMPNTYGVYTAVDHQGPVYPHGQIWDKQIHTDKKPELHCLAPFTCKNNPPGQMFVRIAPNLTDTFNATPTFSEIITYADFWWKGTLKMKIKLRPPHQWNIATVLGAAVNIGDAARFVPNRLGQLEFPVINGRIVPSTVY

SEQ ID NO: 118 - DNA, 89 bp, synthetic construct
  source 1..89: mol_type = other DNA
catggagataattaaaatgataaccatctcgcaaataaataagtattttactgttttcgtaacagttttgtaataaaaaaacctataaa

SEQ ID NO: 119 - DNA, 580 bp, synthetic construct
  source 1..580: mol_type = other DNA
ggtacctctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccac

SEQ ID NO: 120 - DNA, 292 bp, synthetic construct
  source 1..292: mol_type = other DNA
gcgaaacacgcacggcgcgcgcacgcagcttagcacaaacgcgtcgttgcacgcgcccaccgctaaccgcaggccaatcggtcggccggcctcatatccgctcaccagccgcgtcctatcgggcgcggcttccgcgcccattttgaataaataaacgataacgccgttggtggcgtgaggcatgtaaaaggttacatcattatcttgttcgccatccggttggtataaatagacgttcatgttggtttttgtttcagttgcaagttggctgcggcgcgcgcagcacctttgc

SEQ ID NO: 121 - DNA, 110 bp, synthetic construct
  source 1..110: mol_type = other DNA
gacctttaattcaacccaacacaatatattatagttaaataagaattattatcaaatcatttgtatattaattaaaatactatactgtaaattacattttatttacaatc

SEQ ID NO: 122 - DNA, 40 bp, synthetic construct
  source 1..40: mol_type = other DNA
ctcgacgaagacttgatcacccgggggatcccctgttaag

SEQ ID NO: 123 - DNA, 42 bp, synthetic construct
  source 1..42: mol_type = other DNA
attccggattattcataccgtcccaccatcgggcgcggatct

SEQ ID NO: 124 - DNA, 42 bp, synthetic construct
  source 1..42: mol_type = other DNA
actccggactactgataccgtcccactttcgggcgcttacct

SEQ ID NO: 125 - DNA, 132 bp, synthetic construct
  source 1..132: mol_type = other DNA
ttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 126 - DNA, 2591 bp, synthetic construct
  source 1..2591: mol_type = other DNA
catggagataattaaaatgataaccatctcgcaaataaataagtattttactgttttcgtaacagttttgtaataaaaaaacctataaaattccggattattcataccgtcccaccatcgggcgcggatctcctgttaagctggcacctccggcaaagagagccaggagaggatataaatatcttgggcctgggaacagtcttgaccaaggagaaccaactaacccttctgacgccgctgcaaaagaacacgacgaagcttacgctgcttatcttcgctctggtaaaaacccatacttatatttctcgccagcagatcaacgctttatagatcaaactaaggacgctaaagattggggggggaaaataggacattatttttttagagctaaaaaggcaattgctccagtattaactgatacaccagatcatccatcaacatcaagaccaacaaaaccaactaaaagaagtaaaccaccacctcatattttcatcaatcttgcaaaaaaaaaaaaagccggtgcaggacaagtaaaaagagacaatcttgcaccaatgagtgatggagcagttcaaccagacggtggtcaacctgctgtcagaaatgaaagagctacaggatctgggaacgggtctggaggcgggggtggtggtggttctgggggtgtggggatttctacgggtactttcaataatcagacggaatttaaatttttggaaaacggatgggtggaaatcacagcaaactcaagcagacttgtacatttaaatatgccagaaagtgaaaattatagaagagtggttgtaaataatatggataaaactgcagttaacggaaacatggctttagatgatattcatgcacaaattgtaacaccttggtcattggttgatgcaaatgcttggggagtttggtttaatccaggagattggcaactaattgttaatactatgagtgagttgcatttagttagttttgaacaagaaatttttaatgttgttttaaagactgtttcagaatctgctactcagccaccaactaaagtttataataatgatttaactgcatcattgatggttgcattagatagtaataatactatgccatttactccagcagctatgagatctgagacattgggtttttatccatggaaaccaaccataccaactccatggagatattattttcaatgggatagaacattaataccatctcatactggaactagtggcacaccaacaaatatataccatggtacagatccagatgatgttcaattttatactattgaaaattctgtgccagtacacttactaagaacaggtgatgaatttgctacaggaacatttttttttgattgtaaaccatgtagactaacacatacatggcaaacaaatagagcattgggcttaccaccatttctaaattctttgcctcaatctgaaggagctactaactttggtgatataggagttcaacaagataaaagacgtggtgtaactcaaatgggaaatacaaactatattactgaagctactattatgagaccagctgaggttggttatagtgcaccatattattcttttgaggcgtctacacaagggccatttaaaacacctattgcagcaggacgggggggagcgcaaacatatgaaaatcaagcagcagatggtgatccaagatatgcatttggtagacaacatggtcaaaaaactaccacaacaggagaaacacctgagagatttacatatatagcacatcaagatacaggaagatatccagaaggagattggattcaaaatattaactttaaccttcctgtaacgaatgataatgtattgctaccaacagatccaattggaggtaaaacaggaattaactatactaatatatttaatacttatggtcctttaactgcattaaataatgtaccaccagtttatccaaatggtcaaatttgggataaagaatttgatactgacttaaaaccaagacttcatgtaaatgcaccatttgtttgtcaaaataattgtcctggtcaattatttgtaaaagttgcgcctaatttaacaaatgaatatgatcctgatgcatctgctaatatgtcaagaattgtaacttactcagatttttggtggaaaggtaaattagtatttaaagctaaactaagagcctctcatacttggaatccaattcaacaaatgagtattaatgtagataaccaatttaactatgtaccaagtaatattggaggtatgaaaattgtatatgaaaaatctcaactagcacctagaaaattatattaactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 127 - DNA, 2540 bp, synthetic construct
  source 1..2540: mol_type = other DNA
catggagataattaaaatgataaccatctcgcaaataaataagtattttactgttttcgtaacagttttgtaataaaaaaacctataaactggcacctccggcaaagagagccaggagaggatataaatatcttgggcctgggaacagtcttgaccaaggagaaccaactaacccttctgacgccgctgcaaaagaacacgacgaagcttacgctgcttatcttcgctctggtaaaaacccatacttatatttctcgccagcagatcaacgctttatagatcaaactaaggacgctaaagattggggggggaaaataggacattatttttttagagctaaaaaggcaattgctccagtattaactgatacaccagatcatccatcaacatcaagaccaacaaaaccaactaaaagaagtaaaccaccacctcatattttcatcaatcttgcaaaaaaaaaaaaagccggtgcaggacaagtaaaaagagacaatcttgcaccaatgagtgatggagcagttcaaccagacggtggtcaacctgctgtcagaaatgaaagagctacaggatctgggaacgggtctggaggcgggggtggtggtggttctgggggtgtggggatttctacgggtactttcaataatcagacggaatttaaatttttggaaaacggatgggtggaaatcacagcaaactcaagcagacttgtacatttaaatatgccagaaagtgaaaattatagaagagtggttgtaaataatatggataaaactgcagttaacggaaacatggctttagatgatattcatgcacaaattgtaacaccttggtcattggttgatgcaaatgcttggggagtttggtttaatccaggagattggcaactaattgttaatactatgagtgagttgcatttagttagttttgaacaagaaatttttaatgttgttttaaagactgtttcagaatctgctactcagccaccaactaaagtttataataatgatttaactgcatcattgatggttgcattagatagtaataatactatgccatttactccagcagctatgagatctgagacattgggtttttatccatggaaaccaaccataccaactccatggagatattattttcaatgggatagaacattaataccatctcatactggaactagtggcacaccaacaaatatataccatggtacagatccagatgatgttcaattttatactattgaaaattctgtgccagtacacttactaagaacaggtgatgaatttgctacaggaacatttttttttgattgtaaaccatgtagactaacacatacatggcaaacaaatagagcattgggcttaccaccatttctaaattctttgcctcaatctgaaggagctactaactttggtgatataggagttcaacaagataaaagacgtggtgtaactcaaatgggaaatacaaactatattactgaagctactattatgagaccagctgaggttggttatagtgcaccatattattcttttgaggcgtctacacaagggccatttaaaacacctattgcagcaggacgggggggagcgcaaacatatgaaaatcaagcagcagatggtgatccaagatatgcatttggtagacaacatggtcaaaaaactaccacaacaggagaaacacctgagagatttacatatatagcacatcaagatacaggaagatatccagaaggagattggattcaaaatattaactttaaccttcctgtaacgaatgataatgtattgctaccaacagatccaattggaggtaaaacaggaattaactatactaatatatttaatacttatggtcctttaactgcattaaataatgtaccaccagtttatccaaatggtcaaatttgggataaagaatttgatactgacttaaaaccaagacttcatgtaaatgcaccatttgtttgtcaaaataattgtcctggtcaattatttgtaaaagttgcgcctaatttaacaaatgaatatgatcctgatgcatctgctaatatgtcaagaattgtaacttactcagatttttggtggaaaggtaaattagtatttaaagctaaactaagagcctctcatacttggaatccaattcaacaaatgagtattaatgtagataaccaatttaactatgtaccaagtaatattggaggtatgaaaattgtatatgaaaaatctcaactagcacctagaaaattatattaactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 128 - DNA, 2540 bp, synthetic construct
  source 1..2540: mol_type = other DNA
catggagataattaaaatgataaccatctcgcaaataaataagtattttactgttttcgtaacagttttgtaataaaaaaacctataaaacggcacctccggcaaagagagccaggagaggatataaatatcttgggcctgggaacagtcttgaccaaggagaaccaactaacccttctgacgccgctgcaaaagaacacgacgaagcttacgctgcttatcttcgctctggtaaaaacccatacttatatttctcgccagcagatcaacgctttatagatcaaactaaggacgctaaagattggggggggaaaataggacattatttttttagagctaaaaaggcaattgctccagtattaactgatacaccagatcatccatcaacatcaagaccaacaaaaccaactaaaagaagtaaaccaccacctcatattttcatcaatcttgcaaaaaaaaaaaaagccggtgcaggacaagtaaaaagagacaatcttgcaccaatgagtgatggagcagttcaaccagacggtggtcaacctgctgtcagaaatgaaagagctacaggatctgggaacgggtctggaggcgggggtggtggtggttctgggggtgtggggatttctacgggtactttcaataatcagacggaatttaaatttttggaaaacggatgggtggaaatcacagcaaactcaagcagacttgtacatttaaatatgccagaaagtgaaaattatagaagagtggttgtaaataatatggataaaactgcagttaacggaaacatggctttagatgatattcatgcacaaattgtaacaccttggtcattggttgatgcaaatgcttggggagtttggtttaatccaggagattggcaactaattgttaatactatgagtgagttgcatttagttagttttgaacaagaaatttttaatgttgttttaaagactgtttcagaatctgctactcagccaccaactaaagtttataataatgatttaactgcatcattgatggttgcattagatagtaataatactatgccatttactccagcagctatgagatctgagacattgggtttttatccatggaaaccaaccataccaactccatggagatattattttcaatgggatagaacattaataccatctcatactggaactagtggcacaccaacaaatatataccatggtacagatccagatgatgttcaattttatactattgaaaattctgtgccagtacacttactaagaacaggtgatgaatttgctacaggaacatttttttttgattgtaaaccatgtagactaacacatacatggcaaacaaatagagcattgggcttaccaccatttctaaattctttgcctcaatctgaaggagctactaactttggtgatataggagttcaacaagataaaagacgtggtgtaactcaaatgggaaatacaaactatattactgaagctactattatgagaccagctgaggttggttatagtgcaccatattattcttttgaggcgtctacacaagggccatttaaaacacctattgcagcaggacgggggggagcgcaaacatatgaaaatcaagcagcagatggtgatccaagatatgcatttggtagacaacatggtcaaaaaactaccacaacaggagaaacacctgagagatttacatatatagcacatcaagatacaggaagatatccagaaggagattggattcaaaatattaactttaaccttcctgtaacgaatgataatgtattgctaccaacagatccaattggaggtaaaacaggaattaactatactaatatatttaatacttatggtcctttaactgcattaaataatgtaccaccagtttatccaaatggtcaaatttgggataaagaatttgatactgacttaaaaccaagacttcatgtaaatgcaccatttgtttgtcaaaataattgtcctggtcaattatttgtaaaagttgcgcctaatttaacaaatgaatatgatcctgatgcatctgctaatatgtcaagaattgtaacttactcagatttttggtggaaaggtaaattagtatttaaagctaaactaagagcctctcatacttggaatccaattcaacaaatgagtattaatgtagataaccaatttaactatgtaccaagtaatattggaggtatgaaaattgtatatgaaaaatctcaactagcacctagaaaattatattaactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 129 - DNA, 2540 bp, synthetic construct
  source 1..2540: mol_type = other DNA
catggagataattaaaatgataaccatctcgcaaataaataagtattttactgttttcgtaacagttttgtaataaaaaaacctataaattggcacctccggcaaagagagccaggagaggatataaatatcttgggcctgggaacagtcttgaccaaggagaaccaactaacccttctgacgccgctgcaaaagaacacgacgaagcttacgctgcttatcttcgctctggtaaaaacccatacttatatttctcgccagcagatcaacgctttatagatcaaactaaggacgctaaagattggggggggaaaataggacattatttttttagagctaaaaaggcaattgctccagtattaactgatacaccagatcatccatcaacatcaagaccaacaaaaccaactaaaagaagtaaaccaccacctcatattttcatcaatcttgcaaaaaaaaaaaaagccggtgcaggacaagtaaaaagagacaatcttgcaccaatgagtgatggagcagttcaaccagacggtggtcaacctgctgtcagaaatgaaagagctacaggatctgggaacgggtctggaggcgggggtggtggtggttctgggggtgtggggatttctacgggtactttcaataatcagacggaatttaaatttttggaaaacggatgggtggaaatcacagcaaactcaagcagacttgtacatttaaatatgccagaaagtgaaaattatagaagagtggttgtaaataatatggataaaactgcagttaacggaaacatggctttagatgatattcatgcacaaattgtaacaccttggtcattggttgatgcaaatgcttggggagtttggtttaatccaggagattggcaactaattgttaatactatgagtgagttgcatttagttagttttgaacaagaaatttttaatgttgttttaaagactgtttcagaatctgctactcagccaccaactaaagtttataataatgatttaactgcatcattgatggttgcattagatagtaataatactatgccatttactccagcagctatgagatctgagacattgggtttttatccatggaaaccaaccataccaactccatggagatattattttcaatgggatagaacattaataccatctcatactggaactagtggcacaccaacaaatatataccatggtacagatccagatgatgttcaattttatactattgaaaattctgtgccagtacacttactaagaacaggtgatgaatttgctacaggaacatttttttttgattgtaaaccatgtagactaacacatacatggcaaacaaatagagcattgggcttaccaccatttctaaattctttgcctcaatctgaaggagctactaactttggtgatataggagttcaacaagataaaagacgtggtgtaactcaaatgggaaatacaaactatattactgaagctactattatgagaccagctgaggttggttatagtgcaccatattattcttttgaggcgtctacacaagggccatttaaaacacctattgcagcaggacgggggggagcgcaaacatatgaaaatcaagcagcagatggtgatccaagatatgcatttggtagacaacatggtcaaaaaactaccacaacaggagaaacacctgagagatttacatatatagcacatcaagatacaggaagatatccagaaggagattggattcaaaatattaactttaaccttcctgtaacgaatgataatgtattgctaccaacagatccaattggaggtaaaacaggaattaactatactaatatatttaatacttatggtcctttaactgcattaaataatgtaccaccagtttatccaaatggtcaaatttgggataaagaatttgatactgacttaaaaccaagacttcatgtaaatgcaccatttgtttgtcaaaataattgtcctggtcaattatttgtaaaagttgcgcctaatttaacaaatgaatatgatcctgatgcatctgctaatatgtcaagaattgtaacttactcagatttttggtggaaaggtaaattagtatttaaagctaaactaagagcctctcatacttggaatccaattcaacaaatgagtattaatgtagataaccaatttaactatgtaccaagtaatattggaggtatgaaaattgtatatgaaaaatctcaactagcacctagaaaattatattaactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 130 - DNA, 3321 bp, synthetic construct
  source 1..3321: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggaagcttcctaggccgccaccatggccccccccgccaagcgcgcccgccgcggctacaagtacctgggccccggcaacagcctggaccagggcgagcccaccaaccccagcgacgccgccgccaaggagcacgacgaggcctacgccgcctacctgcgcagcggcaagaacccctacctgtacttcagccccgccgaccagcgcttcatcgaccagaccaaggacgccaaggactggggcggcaagatcggccactacttcttccgcgccaagaaggccatcgcccccgtgctgaccgacacccccgaccaccccagcaccagccgccccaccaagcccaccaagcgcagcaagccccccccccacatcttcatcaacctggccaagaagaagaaggccggcgccggccaggtgaagcgcgacaacctggcccccatgagcgacggcgccgtgcagcccgacggcggccagcccgccgtgcgcaacgagcgcgccaccggcagcggcaacggcagcggcggcggcggcggcggcggcagcggcggcgtgggcatcagcaccggcaccttcaacaaccagaccgagttcaagttcctggagaacggctgggtggagatcaccgccaacagcagccgcctggtgcacctgaacatgcccgagagcgagaactaccgccgcgtggtggtgaacaacatggacaagaccgccgtgaacggcaacatggccctggacgacatccacgcccagatcgtgaccccctggagcctggtggacgccaacgcctggggcgtgtggttcaaccccggcgactggcagctgatcgtgaacaccatgagcgagctgcacctggtgagcttcgagcaggagatcttcaacgtggtgctgaagaccgtgagcgagagcgccacccagccccccaccaaggtgtacaacaacgacctgaccgccagcctgatggtggccctggacagcaacaacaccatgcccttcacccccgccgccatgcgcagcgagaccctgggcttctacccctggaagcccaccatccccaccccctggcgctactacttccagtgggaccgcaccctgatccccagccacaccggcaccagcggcacccccaccaacatctaccacggcaccgaccccgacgacgtgcagttctacaccatcgagaacagcgtgcccgtgcacctgctgcgcaccggcgacgagttcgccaccggcaccttcttcttcgactgcaagccctgccgcctgacccacacctggcagaccaaccgcgccctgggcctgccccccttcctgaacagcctgccccagagcgagggcgccaccaacttcggcgacatcggcgtgcagcaggacaagcgccgcggcgtgacccagatgggcaacaccaactacatcaccgaggccaccatcatgcgccccgccgaggtgggctacagcgccccctactacagcttcgaggccagcacccagggccccttcaagacccccatcgccgccggccgcggcggcgcccagacctacgagaaccaggccgccgacggcgacccccgctacgccttcggccgccagcacggccagaagaccaccaccaccggcgagacccccgagcgcttcacctacatcgcccaccaggacaccggccgctaccccgagggcgactggatccagaacatcaacttcaacctgcccgtgaccaacgacaacgtgctgctgcccaccgaccccatcggcggcaagaccggcatcaactacaccaacatcttcaacacctacggccccctgaccgccctgaacaacgtgccccccgtgtaccccaacggccagatctgggacaaggagttcgacaccgacctgaagccccgcctgcacgtgaacgcccccttcgtgtgccagaacaactgccccggccagctgttcgtgaaggtggcccccaacctgaccaacgagtacgaccccgacgccagcgccaacatgagccgcatcgtgacctacagcgacttctggtggaagggcaagctggtgttcaaggccaagctgcgcgccagccacacctggaaccccatccagcagatgagcatcaacgtggacaaccagttcaactacgtgcccagcaacatcggcggcatgaagatcgtgtacgagaagagccagctggccccccgcaagctgtactaataactcgagcatgcatctagaggtacatctagatagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 131 - DNA, 2531 bp, synthetic construct
  source 1..2531: mol_type = other DNA
atcatggagataattaaaatgataaccatctcgcaaataaataagtattttactgttttcgtaacagttttgtaataaaaaaacctataaatattccctcgacgaagacttgatcacccgggggatcccctgttaagctggctccagctattagaaaagccagaggttacaacttcctaggacccttcaatcaagacttcaacaaagaaccaactaatccatcagacaacgctgcaaaacaacacgatttggaatacaacaaactaatcaaccaaggacacaatccttattggtactacaacaaagctgacgaagacttcatcaaagcaacagatcaagcaccagactggggaggaaaatttggcaacttcatcttcagagccaaaaaacacatcgctccagaactggcaccaccagcaaaaaagaaaagcaaaaccaaacacagtgaaccagaattcagccacaaacacatcaaaccaggcaccaaaagaggtaagccttttcatatttttgtaaaccttgctagaaaaagagcccgcatgtcagaaccagctaatgatacaaatgaacaaccagacaactcccctgttgaacagggtgctggtcaaattggaggaggtggaggtggaggtggaagcggtgtcgggcacagcactggtgattataataataggactgagtttatttatcatggtgatgaagtcacaattatttgccactctacaagactggttcacatcaatatgtcagacagggaagactacatcatctatgaaacagacagaggaccactctttcctaccactcaggacctgcagggtagagacactctaaatgactcttaccatgccaaagtagaaacaccatggaaactactccatgcaaacagctggggctgctggttttcaccagcagacttccaacaaatgatcaccacatgcagagacatagcaccaataaaaatgcaccaaaaaatagaaaacattgtcatcaaaacagtcagtaaaacaggcacaggagaaacagaaacaaccaactacaacaatgacctcacagcactcctacaaattgcacaagacaacagtaacctactaccatgggctgcagataacttttatatagactcagtaggttacgttccatggagagcatgcaaactaccaacctactgctaccacgtagacacttggaatacaattgacataaaccaagcagacacaccaaaccaatggagagaaatcaaaaaaggcatccaatgggacaatatccaattcacaccactagaaactatgataaacattgacttactaagaacaggagatgcctgggaatctggtaactacaatttccacacaaaaccaacaaacctagcttaccattggcaatcacaaagacacacaggcagctgtcacccaacagtagcacctctagttgaaagaggacaaggaaccaacatacaatcagtaaactgttggcaatggggagacagaaacaatccaagctctgcatcaaccagagtatccaatatacatattggatactcatttccagaatggcaaatccactactcaacaggaggaccagtaattaatccaggcagtgcattctcacaagcaccatggggctcaacaactgaaggcaccagactaacccaaggtgcatctgaaaaagccatctatgactggtcccatggagatgaccaaccaggagccagagaaacctggtggcaaaacaaccaacatgtaacaggacaaactgactgggcaccaaaaaatgcacacacctcagaactcaacaacaatgtaccagcagccacacacttctggaaaaacagctatcacaacaccttctcaccattcactgcagtagatgatcatggaccacaatatccatggggagccatctggggaaaatacccagacaccacacacaaaccaatgatgtcagctcacgcaccattcctacttcatggaccacctggacaactctttgtaaaactagcaccaaactatacagacacacttgacaacggaggtgtaacacatcccagaatcgtcacatatggaaccttctggtggtcaggacaactcatctttaaaggaaaactacgcactccaagacaatggaatacctacaacctaccaagcctagacaaaagagaaaccatgaaaaacacagtaccaaatgaagttggtcactttgaactaccatacatgccaggaagatgtctaccaaactacacattgtaactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 132 - DNA, 2531 bp, synthetic construct
  source 1..2531: mol_type = other DNA
atcatggagataattaaaatgataaccatctcgcaaataaataagtattttactgttttcgtaacagttttgtaataaaaaaacctataaatactccggactactgataccgtcccactttcgggcgcttacctgccgccacgccagctattagaaaagccagaggatacaacttcctaggacccttcaatcaagacttcaacaaagaaccaactaatccatcagacaacgctgcaaaacaacacgatttggaatacaacaaactaatcaaccaaggacacaatccttattggtactacaacaaagctgacgaagacttcatcaaagcaacagatcaagcaccagactggggaggaaaatttggcaacttcatcttcagagccaaaaaacacatcgctccagaactggcaccaccagcaaaaaagaaaagcaaaaccaaacacagtgaaccagaattcagccacaaacacatcaaaccaggcaccaaaagaggtaagccttttcatatttttgtaaaccttgctagaaaaagagcccgcatgtcagaaccagctaatgatacaaatgaacaaccagacaactcccctgttgaacagggtgctggtcaaattggaggaggtggaggtggaggtggaagcggtgtcgggcacagcactggtgattataataataggactgagtttatttatcatggtgatgaagtcacaattatttgccactctacaagactggttcacatcaatatgtcagacagggaagactacatcatctatgaaacagacagaggaccactctttcctaccactcaggacctgcagggtagagacactctaaatgactcttaccatgccaaagtagaaacaccatggaaactactccatgcaaacagctggggctgctggttttcaccagcagacttccaacaaatgatcaccacatgcagagacatagcaccaataaaaatgcaccaaaaaatagaaaacattgtcatcaaaacagtcagtaaaacaggcacaggagaaacagaaacaaccaactacaacaatgacctcacagcactcctacaaattgcacaagacaacagtaacctactaccatgggctgcagataacttttatatagactcagtaggttacgttccatggagagcatgcaaactaccaacctactgctaccacgtagacacttggaatacaattgacataaaccaagcagacacaccaaaccaatggagagaaatcaaaaaaggcatccaatgggacaatatccaattcacaccactagaaactatgataaacattgacttactaagaacaggagatgcctgggaatctggtaactacaatttccacacaaaaccaacaaacctagcttaccattggcaatcacaaagacacacaggcagctgtcacccaacagtagcacctctagttgaaagaggacaaggaaccaacatacaatcagtaaactgttggcaatggggagacagaaacaatccaagctctgcatcaaccagagtatccaatatacatattggatactcatttccagaatggcaaatccactactcaacaggaggaccagtaattaatccaggcagtgcattctcacaagcaccatggggctcaacaactgaaggcaccagactaacccaaggtgcatctgaaaaagccatctatgactggtcccatggagatgaccaaccaggagccagagaaacctggtggcaaaacaaccaacatgtaacaggacaaactgactgggcaccaaaaaatgcacacacctcagaactcaacaacaatgtaccagcagccacacacttctggaaaaacagctatcacaacaccttctcaccattcactgcagtagatgatcatggaccacaatatccatggggagccatctggggaaaatacccagacaccacacacaaaccaatgatgtcagctcacgcaccattcctacttcatggaccacctggacaactctttgtaaaactagcaccaaactatacagacacacttgacaacggaggtgtaacacatcccagaatcgtcacatatggaaccttctggtggtcaggacaactcatctttaaaggaaaactacgcactccaagacaatggaatacctacaacctaccaagcctagacaaaagagaaaccatgaaaaacacagtaccaaatgaagttggtcactttgaactaccatacatgccaggaagatgtctaccaaactacacattgtaactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 133 - DNA, 3261 bp, synthetic construct
  source 1..3261: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggaagcttcctaggccgccaccatgcccgccatccgcaaggcccgcggctacaacttcctgggccccttcaaccaggacttcaacaaggagcccaccaaccccagcgacaacgccgccaagcagcacgacctggagtacaacaagctgatcaaccagggccacaacccctactggtactacaacaaggccgacgaggacttcatcaaggccaccgaccaggcccccgactggggcggcaagttcggcaacttcatcttccgcgccaagaagcacatcgcccccgagctggccccccccgccaagaagaagagcaagaccaagcacagcgagcccgagttcagccacaagcacatcaagcccggcaccaagcgcggcaagcccttccacatcttcgtgaacctggcccgcaagcgcgcccgcatgagcgagcccgccaacgacaccaacgagcagcccgacaacagccccgtggagcagggcgccggccagatcggcggcggcggcggcggcggcggcagcggcgtgggccacagcaccggcgactacaacaaccgcaccgagttcatctaccacggcgacgaggtgaccatcatctgccacagcacccgcctggtgcacatcaacatgagcgaccgcgaggactacatcatctacgagaccgaccgcggccccctgttccccaccacccaggacctgcagggccgcgacaccctgaacgacagctaccacgccaaggtggagaccccctggaagctgctgcacgccaacagctggggctgctggttcagccccgccgacttccagcagatgatcaccacctgccgcgacatcgcccccatcaagatgcaccagaagatcgagaacatcgtgatcaagaccgtgagcaagaccggcaccggcgagaccgagaccaccaactacaacaacgacctgaccgccctgctgcagatcgcccaggacaacagcaacctgctgccctgggccgccgacaacttctacatcgacagcgtgggctacgtgccctggcgcgcctgcaagctgcccacctactgctaccacgtggacacctggaacaccatcgacatcaaccaggccgacacccccaaccagtggcgcgagatcaagaagggcatccagtgggacaacatccagttcacccccctggagaccatgatcaacatcgacctgctgcgcaccggcgacgcctgggagagcggcaactacaacttccacaccaagcccaccaacctggcctaccactggcagagccagcgccacaccggcagctgccaccccaccgtggcccccctggtggagcgcggccagggcaccaacatccagagcgtgaactgctggcagtggggcgaccgcaacaaccccagcagcgccagcacccgcgtgagcaacatccacatcggctacagcttccccgagtggcagatccactacagcaccggcggccccgtgatcaaccccggcagcgccttcagccaggccccctggggcagcaccaccgagggcacccgcctgacccagggcgccagcgagaaggccatctacgactggagccacggcgacgaccagcccggcgcccgcgagacctggtggcagaacaaccagcacgtgaccggccagaccgactgggcccccaagaacgcccacaccagcgagctgaacaacaacgtgcccgccgccacccacttctggaagaacagctaccacaacaccttcagccccttcaccgccgtggacgaccacggcccccagtacccctggggcgccatctggggcaagtaccccgacaccacccacaagcccatgatgagcgcccacgcccccttcctgctgcacggcccccccggccagctgttcgtgaagctggcccccaactacaccgacaccctggacaacggcggcgtgacccacccccgcatcgtgacctacggcaccttctggtggagcggccagctgatcttcaagggcaagctgcgcaccccccgccagtggaacacctacaacctgcccagcctggacaagcgcgagaccatgaagaacaccgtgcccaacgaggtgggccacttcgagctgccctacatgcccggccgctgcctgcccaactacaccctgtaataactcgagcatgcatctagaggtacatctagatagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 134 - DNA, 2516 bp, synthetic construct
  source 1..2516: mol_type = other DNA
atcatggagataattaaaatgataaccatctcgcaaataaataagtattttactgttttcgtaacagttttgtaataaaaaaacctataaatactccggactactgataccgtcccactttcgggcgcttacctgccgccacgccagctattagaaaagccagaggacccttcaatcaagacttcaacaaagaaccaactaatccatcagacaacgctgcaaaacaacacgatttggaatacaacaaactaatcaaccaaggacacaatccttattggtactacaacaaagctgacgaagacttcatcaaagcaacagatcaagcaccagactggggaggaaaatttggcaacttcatcttcagagccaaaaaacacatcgctccagaactggcaccaccagcaaaaaagaaaagcaaaaccaaacacagtgaaccagaattcagccacaaacacatcaaaccaggcaccaaaagaggtaagccttttcatatttttgtaaaccttgctagaaaaagagcccgcatgtcagaaccagctaatgatacaaatgaacaaccagacaactcccctgttgaacagggtgctggtcaaattggaggaggtggaggtggaggtggaagcggtgtcgggcacagcactggtgattataataataggactgagtttatttatcatggtgatgaagtcacaattatttgccactctacaagactggttcacatcaatatgtcagacagggaagactacatcatctatgaaacagacagaggaccactctttcctaccactcaggacctgcagggtagagacactctaaatgactcttaccatgccaaagtagaaacaccatggaaactactccatgcaaacagctggggctgctggttttcaccagcagacttccaacaaatgatcaccacatgcagagacatagcaccaataaaaatgcaccaaaaaatagaaaacattgtcatcaaaacagtcagtaaaacaggcacaggagaaacagaaacaaccaactacaacaatgacctcacagcactcctacaaattgcacaagacaacagtaacctactaccatgggctgcagataacttttatatagactcagtaggttacgttccatggagagcatgcaaactaccaacctactgctaccacgtagacacttggaatacaattgacataaaccaagcagacacaccaaaccaatggagagaaatcaaaaaaggcatccaatgggacaatatccaattcacaccactagaaactatgataaacattgacttactaagaacaggagatgcctgggaatctggtaactacaatttccacacaaaaccaacaaacctagcttaccattggcaatcacaaagacacacaggcagctgtcacccaacagtagcacctctagttgaaagaggacaaggaaccaacatacaatcagtaaactgttggcaatggggagacagaaacaatccaagctctgcatcaaccagagtatccaatatacatattggatactcatttccagaatggcaaatccactactcaacaggaggaccagtaattaatccaggcagtgcattctcacaagcaccatggggctcaacaactgaaggcaccagactaacccaaggtgcatctgaaaaagccatctatgactggtcccatggagatgaccaaccaggagccagagaaacctggtggcaaaacaaccaacatgtaacaggacaaactgactgggcaccaaaaaatgcacacacctcagaactcaacaacaatgtaccagcagccacacacttctggaaaaacagctatcacaacaccttctcaccattcactgcagtagatgatcatggaccacaatatccatggggagccatctggggaaaatacccagacaccacacacaaaccaatgatgtcagctcacgcaccattcctacttcatggaccacctggacaactctttgtaaaactagcaccaaactatacagacacacttgacaacggaggtgtaacacatcccagaatcgtcacatatggaaccttctggtggtcaggacaactcatctttaaaggaaaactacgcactccaagacaatggaatacctacaacctaccaagcctagacaaaagagaaaccatgaaaaacacagtaccaaatgaagttggtcactttgaactaccatacatgccaggaagatgtctaccaaactacacattgtaactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 135 - DNA, 3246 bp, synthetic construct
  source 1..3246: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggaagcttcctaggccgccaccatgcccgccatccgcaaggcccgcggccccttcaaccaggacttcaacaaggagcccaccaaccccagcgacaacgccgccaagcagcacgacctggagtacaacaagctgatcaaccagggccacaacccctactggtactacaacaaggccgacgaggacttcatcaaggccaccgaccaggcccccgactggggcggcaagttcggcaacttcatcttccgcgccaagaagcacatcgcccccgagctggccccccccgccaagaagaagagcaagaccaagcacagcgagcccgagttcagccacaagcacatcaagcccggcaccaagcgcggcaagcccttccacatcttcgtgaacctggcccgcaagcgcgcccgcatgagcgagcccgccaacgacaccaacgagcagcccgacaacagccccgtggagcagggcgccggccagatcggcggcggcggcggcggcggcggcagcggcgtgggccacagcaccggcgactacaacaaccgcaccgagttcatctaccacggcgacgaggtgaccatcatctgccacagcacccgcctggtgcacatcaacatgagcgaccgcgaggactacatcatctacgagaccgaccgcggccccctgttccccaccacccaggacctgcagggccgcgacaccctgaacgacagctaccacgccaaggtggagaccccctggaagctgctgcacgccaacagctggggctgctggttcagccccgccgacttccagcagatgatcaccacctgccgcgacatcgcccccatcaagatgcaccagaagatcgagaacatcgtgatcaagaccgtgagcaagaccggcaccggcgagaccgagaccaccaactacaacaacgacctgaccgccctgctgcagatcgcccaggacaacagcaacctgctgccctgggccgccgacaacttctacatcgacagcgtgggctacgtgccctggcgcgcctgcaagctgcccacctactgctaccacgtggacacctggaacaccatcgacatcaaccaggccgacacccccaaccagtggcgcgagatcaagaagggcatccagtgggacaacatccagttcacccccctggagaccatgatcaacatcgacctgctgcgcaccggcgacgcctgggagagcggcaactacaacttccacaccaagcccaccaacctggcctaccactggcagagccagcgccacaccggcagctgccaccccaccgtggcccccctggtggagcgcggccagggcaccaacatccagagcgtgaactgctggcagtggggcgaccgcaacaaccccagcagcgccagcacccgcgtgagcaacatccacatcggctacagcttccccgagtggcagatccactacagcaccggcggccccgtgatcaaccccggcagcgccttcagccaggccccctggggcagcaccaccgagggcacccgcctgacccagggcgccagcgagaaggccatctacgactggagccacggcgacgaccagcccggcgcccgcgagacctggtggcagaacaaccagcacgtgaccggccagaccgactgggcccccaagaacgcccacaccagcgagctgaacaacaacgtgcccgccgccacccacttctggaagaacagctaccacaacaccttcagccccttcaccgccgtggacgaccacggcccccagtacccctggggcgccatctggggcaagtaccccgacaccacccacaagcccatgatgagcgcccacgcccccttcctgctgcacggcccccccggccagctgttcgtgaagctggcccccaactacaccgacaccctggacaacggcggcgtgacccacccccgcatcgtgacctacggcaccttctggtggagcggccagctgatcttcaagggcaagctgcgcaccccccgccagtggaacacctacaacctgcccagcctggacaagcgcgagaccatgaagaacaccgtgcccaacgaggtgggccacttcgagctgccctacatgcccggccgctgcctgcccaactacaccctgtaataactcgagcatgcatctagaggtacatctagatagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 136 - DNA, 2591 bp, synthetic construct
  source 1..2591: mol_type = other DNA
catggagataattaaaatgataaccatctcgcaaataaataagtattttactgttttcgtaacagttttgtaataaaaaaacctataaaattccggattattcataccgtcccaccatcgggcgcggatctcctgttaagctggcacctccggcaaagagagccaggagaggatataaatatcttgggcctgggaacagtcttgaccaaggagaaccaactaacccttctgacgccgctgcaaaagaacacgacgaagcttacgctgcttatcttcgctctggtaaaaacccatacttatatttctcgccagcagatcaacgctttatagatcaaactaaggacgctaaagattggggggggaaaataggacattatttttttagagctaaaaaggcaattgctccagtattaactgatacaccagatcatccatcaacatcaagaccaacaaaaccaactaaaagaagtaaaccaccacctcatattttcatcaatcttgcaaaaaaaaaaaaagccggtgcaggacaagtaaaaagagacaatcttgcaccaatgagtgatggagcagttcaaccagacggtggtcaacctgctgtcagaaatgaaagagctacaggatctgggaacgggtctggaggcgggggtggtggtggttctgggggtgtggggatttctacgggtactttcaataatcagacggaatttaaatttttggaaaacggatgggtggaaatcacagcaaactcaagcagacttgtacatttaaatatgccagaaagtgaaaattataaaagagtagttgtaaataatatggataaaactgcagttaaaggaaacatggctttagatgatattcatgtacaaattgtaacaccttggtcattggttgatgcaaatgcttggggagtttggtttaatccaggagattggcaactaattgttaatactatgagtgagttgcatttagttagttttgaacaagaaatttttaatgttgttttaaagactgtttcagaatctgctactcagccaccaactaaagtttataataatgatttaactgcatcattgatggttgcattagatagtaataatactatgccatttactccagcagctatgagatctgagacattgggtttttatccatggaaaccaaccataccaactccatggagatattattttcaatgggatagaacattaataccatctcatactggaactagtggcacaccaacaaatatataccatggtacagatccagatgatgttcaattttatactattgaaaattctgtgccagtacacttactaagaacaggtgatgaatttgctacaggaacatttttttttgattgtaaaccatgtagactaacacatacatggcaaacaaatagagcattgggcttaccaccatttttaaattctttgcctcaatctgaaggagctactaactttggtgatataggagttcaacaagataaaagacgtggtgtaactcaaatgggaaatacaaactatattactgaagctactattatgagaccagctgaggttggttatagtgcaccatattattcttttgaggcgtctacacaagggccatttaaaacacctattgcagcaggacgggggggagcgcaaacagatgaaaatcaagcagcagatggtgatccaagatatgcatttggtagacaacatggtcaaaaaactaccacaacaggagaaacacctgagagatttacatatatagcacatcaagatacaggaagatatccagaaggagattggattcaaaatattaactttaaccttcctgtaacaaatgataatgtattgctaccaacagatccaattggaggtaaaacaggaattaactatactaatatatttaatacttatggtcctttaactgcattaaataatgtaccaccagtttatccaaatggtcaaatttgggataaagaatttgatactgacttaaaaccaagacttcatgtaaatgcaccatttgtttgtcaaaataattgtcctggtcaattatttgtaaaagttgcgcctaatttaacaaatgaatatgatcctgatgcatctgctaatatgtcaagaattgtaacttactcagatttttggtggaaaggtaaattagtatttaaagctaaactaagagcctctcatacttggaatccaattcaacaaatgagtattaatgtagataaccaatttaactatgtaccaagtaatattggagctatgaaaattgtatatgaaaaatctcaactagcacctagaaaattatattaactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 137 - DNA, 2597 bp, synthetic construct
  source 1..2597: mol_type = other DNA
atcatggagataattaaaatgataaccatctcgcaaataaataagtattttactgttttcgtaacagttttgtaataaaaaaacctataaatactccggactactgataccgtcccactttcgggcgcttacctgccgccacggcgcctccagctaaaagagctaaaagaggctacaagtacctgggaccagggaacagccttgaccaaggagaaccaaccaatccatctgacgccgctgccaaagagcacgacgaggcctacgatcaatacatcaaatctggaaaaaatccttacctgtacttctctgctgctgatcaacgctttattgaccaaaccaaggacgccaaagactggggaggcaaggttggtcactacttttttagaaccaagcgcgcttttgcacctaagcttgctactgactctgaacctggaacttctggtgtaagcagagctggtaaacgcactagaccacctgcttacatttttattaaccaagccagagctaaaaaaaaacttacttcttctgctgcacagcaaagcagtcaaaccatgagtgatggcaccagccaacctgacagcggaaacgctgtccactcagctgcaagagttgaacgagcagctgacggccctggaggctctgggggtgggggctctggcgggggtggggttggtgtttctactgggtcttatgataatcaaacgcattatagattcttgggtgacggctgggtagaaattactgcactagcaactagactagtacatttaaacatgcctaaatcagaaaactattgcagaatcagagttcacaatacaacagacacatcagtcaaaggcaacatggcaaaagatgatgctcatgagcaaatttggacaccatggagcttggtggatgctaatgcttggggagtttggctccagccaagtgactggcaatacatttgcaacaccatgagccagcttaacttggtatcacttgatcaagaaatattcaatgtagtgctgaaaactgttacagagcaagacttaggaggtcaagctataaaaatatacaacaatgaccttacagcttgcatgatggttgcagtagactcaaacaacattttgccatacacacctgcagcaaactcaatggaaacacttggtttctacccctggaaaccaaccatagcatcaccatacaggtactatttttgcgttgacagagatctttcagtgacctacgaaaatcaagaaggcacagttgaacataatgtgatgggaacaccaaaaggaatgaattctcaattttttaccattgagaacacacaacaaatcacattgctcagaacaggggacgaatttgccacaggtacttactactttgacacaaattcagttaaactcacacacacgtggcaaaccaaccgtcaacttggacagcctccactgctgtcaacctttcctgaagctgacactgatgcaggtacacttactgctcaagggagcagacatggaacaacacaaatgggggttaactgggtgagtgaagcaatcagaaccagacctgctcaagtaggattttgtcaaccacacaatgactttgaagccagcagagctggaccatttgctgccccaaaagttccagcagatattactcaaggagtagacaaagaagccaatggcagtgttagatacagttatggcaaacagcatggtgaaaattgggcttcacatggaccagcaccagagcgctacacatgggatgaaacaagctttggttcaggtagagacaccaaagatggttttattcaatcagcaccactagttgttccaccaccactaaatggcattcttacaaatgcaaaccctattgggactaaaaatgacattcatttttcaaatgtttttaacagctatggtccactaactgcattttcacacccaagtcctgtataccctcaaggacaaatatgggacaaagaactagatcttgaacacaaacctagacttcacataactgctccatttgtttgtaaaaacaatgcacctggacaaatgttggttagattaggaccaaacctaactgaccaatatgatccaaacggagccacactttctagaattgttacatacggtacatttttctggaaaggaaaactaaccatgagagcaaaacttagagctaacaccacttggaacccagtgtaccaagtaagtgctgaagacaatggcaactcatacatgagtgtaactaaatggttaccaactgctactggaaacatgcagtctgtgccgcttataacaagacctgttgctagaaatacttactaactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 138 - DNA, 2615 bp, synthetic construct
  source 1..2615: mol_type = other DNA
atcatggagataattaaaatgataaccatctcgcaaataaataagtattttactgttttcgtaacagttttgtaataaaaaaacctataaatactccggactactgataccgtcccactttcgggcgcttacctgccgccacggcacctccagctaaaagagctaaaagaggctacaagtacctgggaccagggaacagccttgaccaaggagaaccaaccaacccttctgacgccgctgccaaagaacacgacgaagcctacgaccaatacatcaaatctggaaaaaatccttacctgtacttctctcctgctgatcaacgcttcattgaccaaaccaaagacgccaaggactggggcggcaaggttggtcactacttttttagaaccaagcgagcttttgcacctaagctttctactgactctgaacctggcacttctggtgtgagcagacctggtaaacgaactaaaccacctgctcacatttttgtaaatcaagccagagctaaaaaaaaacgcgcttctcttgctgcacagcagaggactctgacaatgagtgatggcaccgaaacaaaccaaccagacactggaatcgctaatgctagagttgagcgatcagctgacggaggtggaagctctgggggtgggggctctggcgggggtgggattggtgtttctactgggacttatgataatcaaacgacttataagtttttgggagatggatgggtagaaataactgcacatgcttctagacttttgcacttgggaatgcctccttcagaaaactactgccgcgtcaccgttcacaataatcaaacaacaggacacggaactaaggtaaagggaaacatggcctatgatgacacacatcaacaaatttggacaccatggagcttggtagatgctaatgcttggggagtttggttccaaccaagtgactggcagttcattcaaaacagcatggaatcgctgaatcttgactcattgagccaagaactatttaatgtagtagtcaaaacagtcactgaacaacaaggagctggccaagatgccattaaagtctataataatgacttgacggcctgtatgatggttgctctggatagtaacaacatactgccttacacacctgcagctcaaacatcagaaacacttggtttctacccatggaaaccaaccgcaccagctccttacagatactactttttcatgcctagacaactcagtgtaacctctagcaactctgctgaaggaactcaaatcacagacaccattggagagccacaggcactaaactctcaattttttactattgagaacaccttgcctattactctcctgcgcacaggtgatgagtttacaactggcacctacatctttaacactgacccacttaaacttactcacacatggcaaaccaacagacacttgggcatgcctccaagaataactgacctaccaacatcagatacagcaacagcatcactaactgcaaatggagacagatttggatcaacacaaacacagaatgtgaactatgtcacagaggctttgcgcaccaggcctgctcagattggcttcatgcaacctcatgacaactttgaagcaaacagaggtggcccatttaaggttccagtggtaccgctagacataacagctggcgaggaccatgatgcaaacggagccatacgatttaactatggcaaacaacatggcgaagattgggccaaacaaggagcagcaccagaaaggtacacatgggatgcaattgatagtgcagctgggagggacacagctagatgctttgtacaaagtgcaccaatatctattccaccaaaccaaaaccagatcttgcagcgagaagacgccatagctggcagaactaacatgcattatactaatgtttttaacagctatggtccacttagtgcatttcctcatccagatcccatttatccaaatggacaaatttgggacaaagaattggacctggaacacaaacctagactacacgtaactgcaccatttgtttgtaaaaacaacccaccaggtcaactatttgttcgcttggggcctaatctgactgaccaatttgacccaaacagcacaactgtttctcgcattgttacatatagcactttttactggaagggtattttgaaattcaaagccaaactaagaccaaatctgacctggaatcctgtataccaagcaaccacagactctgttgccaattcttacatgaatgttaagaaatggctcccatctgcaactggcaacatgcactctgatccattgatttgtagacctgtgcctcacatgacatactaactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 139 - DNA, 2546 bp, synthetic construct
  source 1..2546: mol_type = other DNA
atcatggagataattaaaatgataaccatctcgcaaataaataagtattttactgttttcgtaacagttttgtaataaaaaaacctataaatactccggactactgataccgtcccactttcgggcgcttacctgccgccacgccagctattagaaaagccagaggttgggtaccacctggatacaacttcctaggacccttcaatcaagacttcaacaaagaaccaactaatccatcagacaacgctgcaaaacaacacgatttggaatacaacaaactaatcaaccaaggacacaatccttattggtactacaacaaagctgacgaagacttcatcaaagcaacagatcaagcaccagactggggaggaaaatttggcaacttcatcttcagagccaaaaaacacatcgctccagaactggcaccaccagcaaaaaagaaaagcaaaaccaaacacagtgaaccagaattcagccacaaacacatcaaaccaggcaccaaaagaggtaagccttttcatatttttgtaaaccttgctagaaaaagagcccgcatgtcagaaccagctaatgatacaaatgaacaaccagacaactcccctgttgaacagggtgctggtcaaattggaggaggtggaggtggaggtggaagcggtgtcgggcacagcactggtgattataataataggactgagtttatttatcatggtgatgaagtcacaattatttgccactctacaagactggttcacatcaatatgtcagacagggaagactacatcatctatgaaacagacagaggaccactctttcctaccactcaggacctgcagggtagagacactctaaatgactcttaccatgccaaagtagaaacaccatggaaactactccatgcaaacagctggggctgctggttttcaccagcagacttccaacaaatgatcaccacatgcagagacatagcaccaataaaaatgcaccaaaaaatagaaaacattgtcatcaaaacagtcagtaaaacaggcacaggagaaacagaaacaaccaactacaacaatgacctcacagcactcctacaaattgcacaagacaacagtaacctactaccatgggctgcagataacttttatatagactcagtaggttacgttccatggagagcatgcaaactaccaacctactgctaccacgtagacacttggaatacaattgacataaaccaagcagacacaccaaaccaatggagagaaatcaaaaaaggcatccaatgggacaatatccaattcacaccactagaaactatgataaacattgacttactaagaacaggagatgcctgggaatctggtaactacaatttccacacaaaaccaacaaacctagcttaccattggcaatcacaaagacacacaggcagctgtcacccaacagtagcacctctagttgaaagaggacaaggaaccaacatacaatcagtaaactgttggcaatggggagacagaaacaatccaagctctgcatcaaccagagtatccaatatacatattggatactcatttccagaatggcaaatccactactcaacaggaggaccagtaattaatccaggcagtgcattctcacaagcaccatggggctcaacaactgaaggcaccagactaacccaaggtgcatctgaaaaagccatctatgactggtcccatggagatgaccaaccaggagccagagaaacctggtggcaaaacaaccaacatgtaacaggacaaactgactgggcaccaaaaaatgcacacacctcagaactcaacaacaatgtaccagcagccacacacttctggaaaaacagctatcacaacaccttctcaccattcactgcagtagatgatcatggaccacaatatccatggggagccatctggggaaaatacccagacaccacacacaaaccaatgatgtcagctcacgcaccattcctacttcatggaccacctggacaactctttgtaaaactagcaccaaactatacagacacacttgacaacggaggtgtaacacatcccagaatcgtcacatatggaaccttctggtggtcaggacaactcatctttaaaggaaaactacgcactccaagacaatggaatacctacaacctaccaagcctagacaaaagagaaaccatgaaaaacacagtaccaaatgaagttggtcactttgaactaccatacatgccaggaagatgtctaccaaactacacattgtaactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 140 - DNA, 2612 bp, synthetic construct
  source 1..2612: mol_type = other DNA
atcatggagataattaaaatgataaccatctcgcaaataaataagtattttactgttttcgtaacagttttgtaataaaaaaacctataaatactccggactactgataccgtcccactttcgggcgcttacctgccgccacggcgcctccagctaaaagagctaaaagaggttgggtgcctcctggctacaagtacctgggaccagggaacagccttgaccaaggagaaccaaccaatccatctgacgccgctgccaaagagcacgacgaggcctacgatcaatacatcaaatctggaaaaaatccttacctgtacttctctgctgctgatcaacgctttattgaccaaaccaaggacgccaaagactggggaggcaaggttggtcactacttttttagaaccaagcgcgcttttgcacctaagcttgctactgactctgaacctggaacttctggtgtaagcagagctggtaaacgcactagaccacctgcttacatttttattaaccaagccagagctaaaaaaaaacttacttcttctgctgcacagcaaagcagtcaaaccatgagtgatggcaccagccaacctgacagcggaaacgctgtccactcagctgcaagagttgaacgagcagctgacggccctggaggctctgggggtgggggctctggcgggggtggggttggtgtttctactgggtcttatgataatcaaacgcattatagattcttgggtgacggctgggtagaaattactgcactagcaactagactagtacatttaaacatgcctaaatcagaaaactattgcagaatcagagttcacaatacaacagacacatcagtcaaaggcaacatggcaaaagatgatgctcatgagcaaatttggacaccatggagcttggtggatgctaatgcttggggagtttggctccagccaagtgactggcaatacatttgcaacaccatgagccagcttaacttggtatcacttgatcaagaaatattcaatgtagtgctgaaaactgttacagagcaagacttaggaggtcaagctataaaaatatacaacaatgaccttacagcttgcatgatggttgcagtagactcaaacaacattttgccatacacacctgcagcaaactcaatggaaacacttggtttctacccctggaaaccaaccatagcatcaccatacaggtactatttttgcgttgacagagatctttcagtgacctacgaaaatcaagaaggcacagttgaacataatgtgatgggaacaccaaaaggaatgaattctcaattttttaccattgagaacacacaacaaatcacattgctcagaacaggggacgaatttgccacaggtacttactactttgacacaaattcagttaaactcacacacacgtggcaaaccaaccgtcaacttggacagcctccactgctgtcaacctttcctgaagctgacactgatgcaggtacacttactgctcaagggagcagacatggaacaacacaaatgggggttaactgggtgagtgaagcaatcagaaccagacctgctcaagtaggattttgtcaaccacacaatgactttgaagccagcagagctggaccatttgctgccccaaaagttccagcagatattactcaaggagtagacaaagaagccaatggcagtgttagatacagttatggcaaacagcatggtgaaaattgggcttcacatggaccagcaccagagcgctacacatgggatgaaacaagctttggttcaggtagagacaccaaagatggttttattcaatcagcaccactagttgttccaccaccactaaatggcattcttacaaatgcaaaccctattgggactaaaaatgacattcatttttcaaatgtttttaacagctatggtccactaactgcattttcacacccaagtcctgtataccctcaaggacaaatatgggacaaagaactagatcttgaacacaaacctagacttcacataactgctccatttgtttgtaaaaacaatgcacctggacaaatgttggttagattaggaccaaacctaactgaccaatatgatccaaacggagccacactttctagaattgttacatacggtacatttttctggaaaggaaaactaaccatgagagcaaaacttagagctaacaccacttggaacccagtgtaccaagtaagtgctgaagacaatggcaactcatacatgagtgtaactaaatggttaccaactgctactggaaacatgcagtctgtgccgcttataacaagacctgttgctagaaatacttactaactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 141 - DNA, 2630 bp, synthetic construct
  source 1..2630: mol_type = other DNA
atcatggagataattaaaatgataaccatctcgcaaataaataagtattttactgttttcgtaacagttttgtaataaaaaaacctataaatactccggactactgataccgtcccactttcgggcgcttacctgccgccacggcacctccagctaaaagagctaaaagaggttgggtgcctcctggctacaagtacctgggaccagggaacagccttgaccaaggagaaccaaccaacccttctgacgccgctgccaaagaacacgacgaagcctacgaccaatacatcaaatctggaaaaaatccttacctgtacttctctcctgctgatcaacgcttcattgaccaaaccaaagacgccaaggactggggcggcaaggttggtcactacttttttagaaccaagcgagcttttgcacctaagctttctactgactctgaacctggcacttctggtgtgagcagacctggtaaacgaactaaaccacctgctcacatttttgtaaatcaagccagagctaaaaaaaaacgcgcttctcttgctgcacagcagaggactctgacaatgagtgatggcaccgaaacaaaccaaccagacactggaatcgctaatgctagagttgagcgatcagctgacggaggtggaagctctgggggtgggggctctggcgggggtgggattggtgtttctactgggacttatgataatcaaacgacttataagtttttgggagatggatgggtagaaataactgcacatgcttctagacttttgcacttgggaatgcctccttcagaaaactactgccgcgtcaccgttcacaataatcaaacaacaggacacggaactaaggtaaagggaaacatggcctatgatgacacacatcaacaaatttggacaccatggagcttggtagatgctaatgcttggggagtttggttccaaccaagtgactggcagttcattcaaaacagcatggaatcgctgaatcttgactcattgagccaagaactatttaatgtagtagtcaaaacagtcactgaacaacaaggagctggccaagatgccattaaagtctataataatgacttgacggcctgtatgatggttgctctggatagtaacaacatactgccttacacacctgcagctcaaacatcagaaacacttggtttctacccatggaaaccaaccgcaccagctccttacagatactactttttcatgcctagacaactcagtgtaacctctagcaactctgctgaaggaactcaaatcacagacaccattggagagccacaggcactaaactctcaattttttactattgagaacaccttgcctattactctcctgcgcacaggtgatgagtttacaactggcacctacatctttaacactgacccacttaaacttactcacacatggcaaaccaacagacacttgggcatgcctccaagaataactgacctaccaacatcagatacagcaacagcatcactaactgcaaatggagacagatttggatcaacacaaacacagaatgtgaactatgtcacagaggctttgcgcaccaggcctgctcagattggcttcatgcaacctcatgacaactttgaagcaaacagaggtggcccatttaaggttccagtggtaccgctagacataacagctggcgaggaccatgatgcaaacggagccatacgatttaactatggcaaacaacatggcgaagattgggccaaacaaggagcagcaccagaaaggtacacatgggatgcaattgatagtgcagctgggagggacacagctagatgctttgtacaaagtgcaccaatatctattccaccaaaccaaaaccagatcttgcagcgagaagacgccatagctggcagaactaacatgcattatactaatgtttttaacagctatggtccacttagtgcatttcctcatccagatcccatttatccaaatggacaaatttgggacaaagaattggacctggaacacaaacctagactacacgtaactgcaccatttgtttgtaaaaacaacccaccaggtcaactatttgttcgcttggggcctaatctgactgaccaatttgacccaaacagcacaactgtttctcgcattgttacatatagcactttttactggaagggtattttgaaattcaaagccaaactaagaccaaatctgacctggaatcctgtataccaagcaaccacagactctgttgccaattcttacatgaatgttaagaaatggctcccatctgcaactggcaacatgcactctgatccattgatttgtagacctgtgcctcacatgacatactaactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 142 - DNA, 2900 bp, synthetic construct
  source 1..2900: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggttgaggaacctgttaagatgagcgacggcgccgtgcagcccgacggcggccagcccgccgtgcgcaacgagcgcgccaccggcagcggcaacggcagcggcggcggcggcggcggcggcagcggcggcgtgggcatcagcaccggcaccttcaacaaccagaccgagttcaagttcctggagaacggctgggtggagatcaccgccaacagcagccgcctggtgcacctgaacatgcccgagagcgagaactaccgccgcgtggtggtgaacaacatggacaagaccgccgtgaacggcaacatggccctggacgacatccacgcccagatcgtgaccccctggagcctggtggacgccaacgcctggggcgtgtggttcaaccccggcgactggcagctgatcgtgaacaccatgagcgagctgcacctggtgagcttcgagcaggagatcttcaacgtggtgctgaagaccgtgagcgagagcgccacccagccccccaccaaggtgtacaacaacgacctgaccgccagcctgatggtggccctggacagcaacaacaccatgcccttcacccccgccgccatgcgcagcgagaccctgggcttctacccctggaagcccaccatccccaccccctggcgctactacttccagtgggaccgcaccctgatccccagccacaccggcaccagcggcacccccaccaacatctaccacggcaccgaccccgacgacgtgcagttctacaccatcgagaacagcgtgcccgtgcacctgctgcgcaccggcgacgagttcgccaccggcaccttcttcttcgactgcaagccctgccgcctgacccacacctggcagaccaaccgcgccctgggcctgccccccttcctgaacagcctgccccagagcgagggcgccaccaacttcggcgacatcggcgtgcagcaggacaagcgccgcggcgtgacccagatgggcaacaccaactacatcaccgaggccaccatcatgcgccccgccgaggtgggctacagcgccccctactacagcttcgaggccagcacccagggccccttcaagacccccatcgccgccggccgcggcggcgcccagacctacgagaaccaggccgccgacggcgacccccgctacgccttcggccgccagcacggccagaagaccaccaccaccggcgagacccccgagcgcttcacctacatcgcccaccaggacaccggccgctaccccgagggcgactggatccagaacatcaacttcaacctgcccgtgaccaacgacaacgtgctgctgcccaccgaccccatcggcggcaagaccggcatcaactacaccaacatcttcaacacctacggccccctgaccgccctgaacaacgtgccccccgtgtaccccaacggccagatctgggacaaggagttcgacaccgacctgaagccccgcctgcacgtgaacgcccccttcgtgtgccagaacaactgccccggccagctgttcgtgaaggtggcccccaacctgaccaacgagtacgaccccgacgccagcgccaacatgagccgcatcgtgacctacagcgacttctggtggaagggcaagctggtgttcaaggccaagctgcgcgccagccacacctggaaccccatccagcagatgagcatcaacgtggacaaccagttcaactacgtgcccagcaacatcggcggcatgaagatcgtgtacgagaagagccagctggccccccgcaagctgtactaataactcgagcatgcatctagagatctagatagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 143 - DNA, 2855 bp, synthetic construct
  source 1..2855: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggttgaggaacctgttaagatgagcgagcccgccaacgacaccaacgagcagcccgacaacagccccgtggagcagggcgccggccagatcggcggcggcggcggcggcggcggcagcggcgtgggccacagcaccggcgactacaacaaccgcaccgagttcatctaccacggcgacgaggtgaccatcatctgccacagcacccgcctggtgcacatcaacatgagcgaccgcgaggactacatcatctacgagaccgaccgcggccccctgttccccaccacccaggacctgcagggccgcgacaccctgaacgacagctaccacgccaaggtggagaccccctggaagctgctgcacgccaacagctggggctgctggttcagccccgccgacttccagcagatgatcaccacctgccgcgacatcgcccccatcaagatgcaccagaagatcgagaacatcgtgatcaagaccgtgagcaagaccggcaccggcgagaccgagaccaccaactacaacaacgacctgaccgccctgctgcagatcgcccaggacaacagcaacctgctgccctgggccgccgacaacttctacatcgacagcgtgggctacgtgccctggcgcgcctgcaagctgcccacctactgctaccacgtggacacctggaacaccatcgacatcaaccaggccgacacccccaaccagtggcgcgagatcaagaagggcatccagtgggacaacatccagttcacccccctggagaccatgatcaacatcgacctgctgcgcaccggcgacgcctgggagagcggcaactacaacttccacaccaagcccaccaacctggcctaccactggcagagccagcgccacaccggcagctgccaccccaccgtggcccccctggtggagcgcggccagggcaccaacatccagagcgtgaactgctggcagtggggcgaccgcaacaaccccagcagcgccagcacccgcgtgagcaacatccacatcggctacagcttccccgagtggcagatccactacagcaccggcggccccgtgatcaaccccggcagcgccttcagccaggccccctggggcagcaccaccgagggcacccgcctgacccagggcgccagcgagaaggccatctacgactggagccacggcgacgaccagcccggcgcccgcgagacctggtggcagaacaaccagcacgtgaccggccagaccgactgggcccccaagaacgcccacaccagcgagctgaacaacaacgtgcccgccgccacccacttctggaagaacagctaccacaacaccttcagccccttcaccgccgtggacgaccacggcccccagtacccctggggcgccatctggggcaagtaccccgacaccacccacaagcccatgatgagcgcccacgcccccttcctgctgcacggcccccccggccagctgttcgtgaagctggcccccaactacaccgacaccctggacaacggcggcgtgacccacccccgcatcgtgacctacggcaccttctggtggagcggccagctgatcttcaagggcaagctgcgcaccccccgccagtggaacacctacaacctgcccagcctggacaagcgcgagaccatgaagaacaccgtgcccaacgaggtgggccacttcgagctgccctacatgcccggccgctgcctgcccaactacaccctgtaataactcgagcatgcatctagagatctagatagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 144 - DNA, 42 bp, synthetic construct
  source 1..42: mol_type = other DNA
gcccgggcaaagcccgggcgtcgggcgacctttggtcgcccg

SEQ ID NO: 145 - protein, 6 aa, synthetic construct
KRARRG

SEQ ID NO: 146 - protein, 6 aa, synthetic construct
KRAKRG

SEQ ID NO: 147 - protein, 4 aa, synthetic construct
KARG

SEQ ID NO: 148 - DNA, 3336 bp, synthetic construct
  source 1..3336: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggaagcttcctaggccgccaccatggccccccccgccaagcgcgcccgccgcggcctggtgccccccggctacaagtacctgggccccggcaacagcctggaccagggcgagcccaccaaccccagcgacgccgccgccaaggagcacgacgaggcctacgccgcctacctgcgcagcggcaagaacccctacctgtacttcagccccgccgaccagcgcttcatcgaccagaccaaggacgccaaggactggggcggcaagatcggccactacttcttccgcgccaagaaggccatcgcccccgtgctgaccgacacccccgaccaccccagcaccagccgccccaccaagcccaccaagcgcagcaagccccccccccacatcttcatcaacctggccaagaagaagaaggccggcgccggccaggtgaagcgcgacaacctggcccccatgagcgacggcgccgtgcagcccgacggcggccagcccgccgtgcgcaacgagcgcgccaccggcagcggcaacggcagcggcggcggcggcggcggcggcagcggcggcgtgggcatcagcaccggcaccttcaacaaccagaccgagttcaagttcctggagaacggctgggtggagatcaccgccaacagcagccgcctggtgcacctgaacatgcccgagagcgagaactaccgccgcgtggtggtgaacaacatggacaagaccgccgtgaacggcaacatggccctggacgacatccacgcccagatcgtgaccccctggagcctggtggacgccaacgcctggggcgtgtggttcaaccccggcgactggcagctgatcgtgaacaccatgagcgagctgcacctggtgagcttcgagcaggagatcttcaacgtggtgctgaagaccgtgagcgagagcgccacccagccccccaccaaggtgtacaacaacgacctgaccgccagcctgatggtggccctggacagcaacaacaccatgcccttcacccccgccgccatgcgcagcgagaccctgggcttctacccctggaagcccaccatccccaccccctggcgctactacttccagtgggaccgcaccctgatccccagccacaccggcaccagcggcacccccaccaacatctaccacggcaccgaccccgacgacgtgcagttctacaccatcgagaacagcgtgcccgtgcacctgctgcgcaccggcgacgagttcgccaccggcaccttcttcttcgactgcaagccctgccgcctgacccacacctggcagaccaaccgcgccctgggcctgccccccttcctgaacagcctgccccagagcgagggcgccaccaacttcggcgacatcggcgtgcagcaggacaagcgccgcggcgtgacccagatgggcaacaccaactacatcaccgaggccaccatcatgcgccccgccgaggtgggctacagcgccccctactacagcttcgaggccagcacccagggccccttcaagacccccatcgccgccggccgcggcggcgcccagacctacgagaaccaggccgccgacggcgacccccgctacgccttcggccgccagcacggccagaagaccaccaccaccggcgagacccccgagcgcttcacctacatcgcccaccaggacaccggccgctaccccgagggcgactggatccagaacatcaacttcaacctgcccgtgaccaacgacaacgtgctgctgcccaccgaccccatcggcggcaagaccggcatcaactacaccaacatcttcaacacctacggccccctgaccgccctgaacaacgtgccccccgtgtaccccaacggccagatctgggacaaggagttcgacaccgacctgaagccccgcctgcacgtgaacgcccccttcgtgtgccagaacaactgccccggccagctgttcgtgaaggtggcccccaacctgaccaacgagtacgaccccgacgccagcgccaacatgagccgcatcgtgacctacagcgacttctggtggaagggcaagctggtgttcaaggccaagctgcgcgccagccacacctggaaccccatccagcagatgagcatcaacgtggacaaccagttcaactacgtgcccagcaacatcggcggcatgaagatcgtgtacgagaagagccagctggccccccgcaagctgtactaataactcgagcatgcatctagaggtacatctagatagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 149 - DNA, 2588 bp, synthetic construct
  source 1..2588: mol_type = other DNA
catggagataattaaaatgataaccatctcgcaaataaataagtattttactgttttcgtaacagttttgtaataaaaaaacctataaaattccggattattcataccgtcccaccatcgggcgcggatctgccgccctggcacctccggcaaagagagccaggagaggatataaatatcttgggcctgggaacagtcttgaccaaggagaaccaactaacccttctgacgccgctgcaaaagaacacgacgaagcttacgctgcttatcttcgctctggtaaaaacccatacttatatttctcgccagcagatcaacgctttatagatcaaactaaggacgctaaagattggggggggaaaataggacattatttttttagagctaaaaaggcaattgctccagtattaactgatacaccagatcatccatcaacatcaagaccaacaaaaccaactaaaagaagtaaaccaccacctcatattttcatcaatcttgcaaaaaaaaaaaaagccggtgcaggacaagtaaaaagagacaatcttgcaccaatgagtgatggagcagttcaaccagacggtggtcaacctgctgtcagaaatgaaagagctacaggatctgggaacgggtctggaggcgggggtggtggtggttctgggggtgtggggatttctacgggtactttcaataatcagacggaatttaaatttttggaaaacggatgggtggaaatcacagcaaactcaagcagacttgtacatttaaatatgccagaaagtgaaaattatagaagagtggttgtaaataatatggataaaactgcagttaacggaaacatggctttagatgatattcatgcacaaattgtaacaccttggtcattggttgatgcaaatgcttggggagtttggtttaatccaggagattggcaactaattgttaatactatgagtgagttgcatttagttagttttgaacaagaaatttttaatgttgttttaaagactgtttcagaatctgctactcagccaccaactaaagtttataataatgatttaactgcatcattgatggttgcattagatagtaataatactatgccatttactccagcagctatgagatctgagacattgggtttttatccatggaaaccaaccataccaactccatggagatattattttcaatgggatagaacattaataccatctcatactggaactagtggcacaccaacaaatatataccatggtacagatccagatgatgttcaattttatactattgaaaattctgtgccagtacacttactaagaacaggtgatgaatttgctacaggaacatttttttttgattgtaaaccatgtagactaacacatacatggcaaacaaatagagcattgggcttaccaccatttctaaattctttgcctcaatctgaaggagctactaactttggtgatataggagttcaacaagataaaagacgtggtgtaactcaaatgggaaatacaaactatattactgaagctactattatgagaccagctgaggttggttatagtgcaccatattattcttttgaggcgtctacacaagggccatttaaaacacctattgcagcaggacgggggggagcgcaaacatatgaaaatcaagcagcagatggtgatccaagatatgcatttggtagacaacatggtcaaaaaactaccacaacaggagaaacacctgagagatttacatatatagcacatcaagatacaggaagatatccagaaggagattggattcaaaatattaactttaaccttcctgtaacgaatgataatgtattgctaccaacagatccaattggaggtaaaacaggaattaactatactaatatatttaatacttatggtcctttaactgcattaaataatgtaccaccagtttatccaaatggtcaaatttgggataaagaatttgatactgacttaaaaccaagacttcatgtaaatgcaccatttgtttgtcaaaataattgtcctggtcaattatttgtaaaagttgcgcctaatttaacaaatgaatatgatcctgatgcatctgctaatatgtcaagaattgtaacttactcagatttttggtggaaaggtaaattagtatttaaagctaaactaagagcctctcatacttggaatccaattcaacaaatgagtattaatgtagataaccaatttaactatgtaccaagtaatattggaggtatgaaaattgtatatgaaaaatctcaactagcacctagaaaattatattaactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 150 - DNA, 2588 bp, synthetic construct
  source 1..2588: mol_type = other DNA
catggagataattaaaatgataaccatctcgcaaataaataagtattttactgttttcgtaacagttttgtaataaaaaaacctataaaattccggattattcataccgtcccaccatcgggcgcggatctgccgccacggcacctccggcaaagagagccaggagaggatataaatatcttgggcctgggaacagtcttgaccaaggagaaccaactaacccttctgacgccgctgcaaaagaacacgacgaagcttacgctgcttatcttcgctctggtaaaaacccatacttatatttctcgccagcagatcaacgctttatagatcaaactaaggacgctaaagattggggggggaaaataggacattatttttttagagctaaaaaggcaattgctccagtattaactgatacaccagatcatccatcaacatcaagaccaacaaaaccaactaaaagaagtaaaccaccacctcatattttcatcaatcttgcaaaaaaaaaaaaagccggtgcaggacaagtaaaaagagacaatcttgcaccaatgagtgatggagcagttcaaccagacggtggtcaacctgctgtcagaaatgaaagagctacaggatctgggaacgggtctggaggcgggggtggtggtggttctgggggtgtggggatttctacgggtactttcaataatcagacggaatttaaatttttggaaaacggatgggtggaaatcacagcaaactcaagcagacttgtacatttaaatatgccagaaagtgaaaattatagaagagtggttgtaaataatatggataaaactgcagttaacggaaacatggctttagatgatattcatgcacaaattgtaacaccttggtcattggttgatgcaaatgcttggggagtttggtttaatccaggagattggcaactaattgttaatactatgagtgagttgcatttagttagttttgaacaagaaatttttaatgttgttttaaagactgtttcagaatctgctactcagccaccaactaaagtttataataatgatttaactgcatcattgatggttgcattagatagtaataatactatgccatttactccagcagctatgagatctgagacattgggtttttatccatggaaaccaaccataccaactccatggagatattattttcaatgggatagaacattaataccatctcatactggaactagtggcacaccaacaaatatataccatggtacagatccagatgatgttcaattttatactattgaaaattctgtgccagtacacttactaagaacaggtgatgaatttgctacaggaacatttttttttgattgtaaaccatgtagactaacacatacatggcaaacaaatagagcattgggcttaccaccatttctaaattctttgcctcaatctgaaggagctactaactttggtgatataggagttcaacaagataaaagacgtggtgtaactcaaatgggaaatacaaactatattactgaagctactattatgagaccagctgaggttggttatagtgcaccatattattcttttgaggcgtctacacaagggccatttaaaacacctattgcagcaggacgggggggagcgcaaacatatgaaaatcaagcagcagatggtgatccaagatatgcatttggtagacaacatggtcaaaaaactaccacaacaggagaaacacctgagagatttacatatatagcacatcaagatacaggaagatatccagaaggagattggattcaaaatattaactttaaccttcctgtaacgaatgataatgtattgctaccaacagatccaattggaggtaaaacaggaattaactatactaatatatttaatacttatggtcctttaactgcattaaataatgtaccaccagtttatccaaatggtcaaatttgggataaagaatttgatactgacttaaaaccaagacttcatgtaaatgcaccatttgtttgtcaaaataattgtcctggtcaattatttgtaaaagttgcgcctaatttaacaaatgaatatgatcctgatgcatctgctaatatgtcaagaattgtaacttactcagatttttggtggaaaggtaaattagtatttaaagctaaactaagagcctctcatacttggaatccaattcaacaaatgagtattaatgtagataaccaatttaactatgtaccaagtaatattggaggtatgaaaattgtatatgaaaaatctcaactagcacctagaaaattatattaactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 151 - DNA, 2588 bp, synthetic construct
  source 1..2588: mol_type = other DNA
catggagataattaaaatgataaccatctcgcaaataaataagtattttactgttttcgtaacagttttgtaataaaaaaacctataaaattccggattattcataccgtcccaccatcgggcgcggatctgccgccttggcacctccggcaaagagagccaggagaggatataaatatcttgggcctgggaacagtcttgaccaaggagaaccaactaacccttctgacgccgctgcaaaagaacacgacgaagcttacgctgcttatcttcgctctggtaaaaacccatacttatatttctcgccagcagatcaacgctttatagatcaaactaaggacgctaaagattggggggggaaaataggacattatttttttagagctaaaaaggcaattgctccagtattaactgatacaccagatcatccatcaacatcaagaccaacaaaaccaactaaaagaagtaaaccaccacctcatattttcatcaatcttgcaaaaaaaaaaaaagccggtgcaggacaagtaaaaagagacaatcttgcaccaatgagtgatggagcagttcaaccagacggtggtcaacctgctgtcagaaatgaaagagctacaggatctgggaacgggtctggaggcgggggtggtggtggttctgggggtgtggggatttctacgggtactttcaataatcagacggaatttaaatttttggaaaacggatgggtggaaatcacagcaaactcaagcagacttgtacatttaaatatgccagaaagtgaaaattatagaagagtggttgtaaataatatggataaaactgcagttaacggaaacatggctttagatgatattcatgcacaaattgtaacaccttggtcattggttgatgcaaatgcttggggagtttggtttaatccaggagattggcaactaattgttaatactatgagtgagttgcatttagttagttttgaacaagaaatttttaatgttgttttaaagactgtttcagaatctgctactcagccaccaactaaagtttataataatgatttaactgcatcattgatggttgcattagatagtaataatactatgccatttactccagcagctatgagatctgagacattgggtttttatccatggaaaccaaccataccaactccatggagatattattttcaatgggatagaacattaataccatctcatactggaactagtggcacaccaacaaatatataccatggtacagatccagatgatgttcaattttatactattgaaaattctgtgccagtacacttactaagaacaggtgatgaatttgctacaggaacatttttttttgattgtaaaccatgtagactaacacatacatggcaaacaaatagagcattgggcttaccaccatttctaaattctttgcctcaatctgaaggagctactaactttggtgatataggagttcaacaagataaaagacgtggtgtaactcaaatgggaaatacaaactatattactgaagctactattatgagaccagctgaggttggttatagtgcaccatattattcttttgaggcgtctacacaagggccatttaaaacacctattgcagcaggacgggggggagcgcaaacatatgaaaatcaagcagcagatggtgatccaagatatgcatttggtagacaacatggtcaaaaaactaccacaacaggagaaacacctgagagatttacatatatagcacatcaagatacaggaagatatccagaaggagattggattcaaaatattaactttaaccttcctgtaacgaatgataatgtattgctaccaacagatccaattggaggtaaaacaggaattaactatactaatatatttaatacttatggtcctttaactgcattaaataatgtaccaccagtttatccaaatggtcaaatttgggataaagaatttgatactgacttaaaaccaagacttcatgtaaatgcaccatttgtttgtcaaaataattgtcctggtcaattatttgtaaaagttgcgcctaatttaacaaatgaatatgatcctgatgcatctgctaatatgtcaagaattgtaacttactcagatttttggtggaaaggtaaattagtatttaaagctaaactaagagcctctcatacttggaatccaattcaacaaatgagtattaatgtagataaccaatttaactatgtaccaagtaatattggaggtatgaaaattgtatatgaaaaatctcaactagcacctagaaaattatattaactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 152 - DNA, 2588 bp, synthetic construct
  source 1..2588: mol_type = other DNA
catggagataattaaaatgataaccatctcgcaaataaataagtattttactgttttcgtaacagttttgtaataaaaaaacctataaaattccggattattcataccgtcccaccatcgggcgcggatctgccgccatcgcacctccggcaaagagagccaggagaggatataaatatcttgggcctgggaacagtcttgaccaaggagaaccaactaacccttctgacgccgctgcaaaagaacacgacgaagcttacgctgcttatcttcgctctggtaaaaacccatacttatatttctcgccagcagatcaacgctttatagatcaaactaaggacgctaaagattggggggggaaaataggacattatttttttagagctaaaaaggcaattgctccagtattaactgatacaccagatcatccatcaacatcaagaccaacaaaaccaactaaaagaagtaaaccaccacctcatattttcatcaatcttgcaaaaaaaaaaaaagccggtgcaggacaagtaaaaagagacaatcttgcaccaatgagtgatggagcagttcaaccagacggtggtcaacctgctgtcagaaatgaaagagctacaggatctgggaacgggtctggaggcgggggtggtggtggttctgggggtgtggggatttctacgggtactttcaataatcagacggaatttaaatttttggaaaacggatgggtggaaatcacagcaaactcaagcagacttgtacatttaaatatgccagaaagtgaaaattatagaagagtggttgtaaataatatggataaaactgcagttaacggaaacatggctttagatgatattcatgcacaaattgtaacaccttggtcattggttgatgcaaatgcttggggagtttggtttaatccaggagattggcaactaattgttaatactatgagtgagttgcatttagttagttttgaacaagaaatttttaatgttgttttaaagactgtttcagaatctgctactcagccaccaactaaagtttataataatgatttaactgcatcattgatggttgcattagatagtaataatactatgccatttactccagcagctatgagatctgagacattgggtttttatccatggaaaccaaccataccaactccatggagatattattttcaatgggatagaacattaataccatctcatactggaactagtggcacaccaacaaatatataccatggtacagatccagatgatgttcaattttatactattgaaaattctgtgccagtacacttactaagaacaggtgatgaatttgctacaggaacatttttttttgattgtaaaccatgtagactaacacatacatggcaaacaaatagagcattgggcttaccaccatttctaaattctttgcctcaatctgaaggagctactaactttggtgatataggagttcaacaagataaaagacgtggtgtaactcaaatgggaaatacaaactatattactgaagctactattatgagaccagctgaggttggttatagtgcaccatattattcttttgaggcgtctacacaagggccatttaaaacacctattgcagcaggacgggggggagcgcaaacatatgaaaatcaagcagcagatggtgatccaagatatgcatttggtagacaacatggtcaaaaaactaccacaacaggagaaacacctgagagatttacatatatagcacatcaagatacaggaagatatccagaaggagattggattcaaaatattaactttaaccttcctgtaacgaatgataatgtattgctaccaacagatccaattggaggtaaaacaggaattaactatactaatatatttaatacttatggtcctttaactgcattaaataatgtaccaccagtttatccaaatggtcaaatttgggataaagaatttgatactgacttaaaaccaagacttcatgtaaatgcaccatttgtttgtcaaaataattgtcctggtcaattatttgtaaaagttgcgcctaatttaacaaatgaatatgatcctgatgcatctgctaatatgtcaagaattgtaacttactcagatttttggtggaaaggtaaattagtatttaaagctaaactaagagcctctcatacttggaatccaattcaacaaatgagtattaatgtagataaccaatttaactatgtaccaagtaatattggaggtatgaaaattgtatatgaaaaatctcaactagcacctagaaaattatattaactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 153 - DNA, 1206 bp, synthetic construct
  source 1..1206: mol_type = other DNA
gtatactccggaatattaatagatgcgaaacacgcacggcgcgcgcacgcagcttagcacaaacgcgtcgttgcacgcgcccaccgctaaccgcaggccaatcggtcggccggcctcatatccgctcaccagccgcgtcctatcgggcgcggcttccgcgcccattttgaataaataaacgataacgccgttggtggcgtgaggcatgtaaaaggttacatcattatcttgttcgccatccggttggtataaatagacgttcatgttggtttttgtttcagttgcaagttggctgcggcgcgcgcagcacctttgctattccggattattcataccgtcccaccatcgggcgcggatctgcctccatgtctggcaaccagtatactgaggaagttatggagggagtaaattggttaaagaaacatgcagaaaatgaagcattttcgtttgtttttaaatgtgacaacgtccaactaaatggaaaggatgttcgctggaacaactataccaaaccaattcaaaatgaagaactaacatctttaattagaggagcacaaacagcaatggatcaaaccgaagaagaagaaatggactgggaatcggaagttgatagtctcgccaaaaagttgcaaagacttagagacacaagcggcaagcaatcctcagagtcaagaccaagttctaactcctctgactccggacgtagtggaccttgcactggaaccgtggagtactccagatacgcctattgcagaaactgcaaatcaacaatcaaaccaacttggcgttactcacaaagacgtgcaagcgagtccgacgtggtccgaaatagaggcagacctgagagccatctttacttccatcatcaccatcaccactgagagctcactagtcgcggccgctttcgaatctagagcctgcagtctcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 154 - DNA, 1188 bp, synthetic construct
  source 1..1188: mol_type = other DNA
gtatactccggaatattaatagatgcgaaacacgcacggcgcgcgcacgcagcttagcacaaacgcgtcgttgcacgcgcccaccgctaaccgcaggccaatcggtcggccggcctcatatccgctcaccagccgcgtcctatcgggcgcggcttccgcgcccattttgaataaataaacgataacgccgttggtggcgtgaggcatgtaaaaggttacatcattatcttgttcgccatccggttggtataaatagacgttcatgttggtttttgtttcagttgcaagttggctgcggcgcgcgcagcacctttgctattccggattattcataccgtcccaccatcgggcgcggatctgcctccctgtctggcaaccagtatactgaggaagttatggagggagtaaattggttaaagaaacatgcagaaaatgaagcattttcgtttgtttttaaatgtgacaacgtccaactaaatggaaaggatgttcgctggaacaactataccaaaccaattcaaaatgaagaactaacatctttaattagaggagcacaaacagcaatggatcaaaccgaagaagaagaaatggactgggaatcggaagttgatagtctcgccaaaaagttgcaaagacttagagacacaagcggcaagcaatcctcagagtcaagaccaagttctaactcctctgactccggacgtagtggaccttgcactggaaccgtggagtactccagatacgcctattgcagaaactgcaaatcaacaatcaaaccaacttggcgttactcacaaagacgtgcaagcgagtccgacgtggtccgaaatagaggcagacctgagagccatctttacttctgagagctcactagtcgcggccgctttcgaatctagagcctgcagtctcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 155 - DNA, 3066 bp, synthetic construct
  source 1..3066: mol_type = other DNA
cgcgtggcctccgcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtggtaccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtagggaagtgttacttctgctctaaaagctgcggaattgtagccgttgaagcttcctaggccgccaccatggccccccccgccaagcgcgcccgccgcggctacaagtacctgggccccggcaacagcctggaccagggcgagcccaccaaccccagcgacgccgccgccaaggagcacgacgaggcctacgccgcctacctgcgcagcggcaagaacccctacctgtacttcagccccgccgaccagcgcttcatcgaccagaccaaggacgccaaggactggggcggcaagatcggccactacttcttccgcgccaagaaggccatcgcccccgtgctgaccgacacccccgaccaccccagcaccagccgccccaccaagcccaccaagcgcagcaagccccccccccacatcttcatcaacctggccaagaagaagaaggccggcgccggccaggtgaagcgcgacaacctggcccccatgagcgacggcgccgtgcagcccgacggcggccagcccgccgtgcgcaacgagcgcgccaccggcagcggcaacggcagcggcggcggcggcggcggcggcagcggcggcgtgggcatcagcaccggcaccttcaacaaccagaccgagttcaagttcctggagaacggctgggtggagatcaccgccaacagcagccgcctggtgcacctgaacatgcccgagagcgagaactaccgccgcgtggtggtgaacaacatggacaagaccgccgtgaacggcaacatggccctggacgacatccacgcccagatcgtgaccccctggagcctggtggacgccaacgcctggggcgtgtggttcaaccccggcgactggcagctgatcgtgaacaccatgagcgagctgcacctggtgagcttcgagcaggagatcttcaacgtggtgctgaagaccgtgagcgagagcgccacccagccccccaccaaggtgtacaacaacgacctgaccgccagcctgatggtggccctggacagcaacaacaccatgcccttcacccccgccgccatgcgcagcgagaccctgggcttctacccctggaagcccaccatccccaccccctggcgctactacttccagtgggaccgcaccctgatccccagccacaccggcaccagcggcacccccaccaacatctaccacggcaccgaccccgacgacgtgcagttctacaccatcgagaacagcgtgcccgtgcacctgctgcgcaccggcgacgagttcgccaccggcaccttcttcttcgactgcaagccctgccgcctgacccacacctggcagaccaaccgcgccctgggcctgccccccttcctgaacagcctgccccagagcgagggcgccaccaacttcggcgacatcggcgtgcagcaggacaagcgccgcggcgtgacccagatgggcaacaccaactacatcaccgaggccaccatcatgcgccccgccgaggtgggctacagcgccccctactacagcttcgaggccagcacccagggccccttcaagacccccatcgccgccggccgcggcggcgcccagacctacgagaaccaggccgccgacggcgacccccgctacgccttcggccgccagcacggccagaagaccaccaccaccggcgagacccccgagcgcttcacctacatcgcccaccaggacaccggccgctaccccgagggcgactggatccagaacatcaacttcaacctgcccgtgaccaacgacaacgtgctgctgcccaccgaccccatcggcggcaagaccggcatcaactacaccaacatcttcaacacctacggccccctgaccgccctgaacaacgtgccccccgtgtaccccaacggccagatctgggacaaggagttcgacaccgacctgaagccccgcctgcacgtgaacgcccccttcgtgtgccagaacaactgccccggccagctgttcgtgaaggtggcccccaacctgaccaacgagtacgaccccgacgccagcgccaacatgagccgcatcgtgacctacagcgacttctggtggaagggcaagctggtgttcaaggccaagctgcgcgccagccacacctggaaccccatccagcagatgagcatcaacgtggacaaccagttcaactacgtgcccagcaacatcggcggcatgaagatcgtgtacgagaagagccagctggccccccgcaagctgtactaataactcgagcatgcatctagaggtacatctagatagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 156 - DNA, 2890 bp, synthetic construct
  source 1..2890: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagcttgattaattaaggccgccaccatgagcgacggcgccgtgcagcccgacggcggccagcccgccgtgcgcaacgagcgcgccaccggcagcggcaacggcagcggcggcggcggcggcggcggcagcggcggcgtgggcatcagcaccggcaccttcaacaaccagaccgagttcaagttcctggagaacggctgggtggagatcaccgccaacagcagccgcctggtgcacctgaacatgcccgagagcgagaactaccgccgcgtggtggtgaacaacatggacaagaccgccgtgaacggcaacatggccctggacgacatccacgcccagatcgtgaccccctggagcctggtggacgccaacgcctggggcgtgtggttcaaccccggcgactggcagctgatcgtgaacaccatgagcgagctgcacctggtgagcttcgagcaggagatcttcaacgtggtgctgaagaccgtgagcgagagcgccacccagccccccaccaaggtgtacaacaacgacctgaccgccagcctgatggtggccctggacagcaacaacaccatgcccttcacccccgccgccatgcgcagcgagaccctgggcttctacccctggaagcccaccatccccaccccctggcgctactacttccagtgggaccgcaccctgatccccagccacaccggcaccagcggcacccccaccaacatctaccacggcaccgaccccgacgacgtgcagttctacaccatcgagaacagcgtgcccgtgcacctgctgcgcaccggcgacgagttcgccaccggcaccttcttcttcgactgcaagccctgccgcctgacccacacctggcagaccaaccgcgccctgggcctgccccccttcctgaacagcctgccccagagcgagggcgccaccaacttcggcgacatcggcgtgcagcaggacaagcgccgcggcgtgacccagatgggcaacaccaactacatcaccgaggccaccatcatgcgccccgccgaggtgggctacagcgccccctactacagcttcgaggccagcacccagggccccttcaagacccccatcgccgccggccgcggcggcgcccagacctacgagaaccaggccgccgacggcgacccccgctacgccttcggccgccagcacggccagaagaccaccaccaccggcgagacccccgagcgcttcacctacatcgcccaccaggacaccggccgctaccccgagggcgactggatccagaacatcaacttcaacctgcccgtgaccaacgacaacgtgctgctgcccaccgaccccatcggcggcaagaccggcatcaactacaccaacatcttcaacacctacggccccctgaccgccctgaacaacgtgccccccgtgtaccccaacggccagatctgggacaaggagttcgacaccgacctgaagccccgcctgcacgtgaacgcccccttcgtgtgccagaacaactgccccggccagctgttcgtgaaggtggcccccaacctgaccaacgagtacgaccccgacgccagcgccaacatgagccgcatcgtgacctacagcgacttctggtggaagggcaagctggtgttcaaggccaagctgcgcgccagccacacctggaaccccatccagcagatgagcatcaacgtggacaaccagttcaactacgtgcccagcaacatcggcggcatgaagatcgtgtacgagaagagccagctggccccccgcaagctgtactaatgactcgagcatgcatctagaggtacatctagatagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 157 - DNA, 189 bp, synthetic construct
  source 1..189: mol_type = other DNA
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgaca

SEQ ID NO: 158 - DNA, 400 bp, synthetic construct
  source 1..400: mol_type = other DNA
ggcctccgcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggt

SEQ ID NO: 159 - DNA, 376 bp, synthetic construct
  source 1..376: mol_type = other DNA
ggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctg

SEQ ID NO: 160 - protein, 34 aa, unidentified
  source 1..34: note = Bufavirus-1
MPAIRKARGWVPPGYNYLGPFNQDFSKKPTNPSD

SEQ ID NO: 161 - protein, 34 aa, unidentified
  source 1..34: note = Cutavirus
MPAIRKARGWVPPGYNFLGPFNQDFNKEPTNPSD

SEQ ID NO: 162 - protein, 35 aa, unidentified
  source 1..35: note = Tusavirus 1
MAPAARPRKGWVPPGYNYLGPGNDLDAGEPTNKSD

SEQ ID NO: 163 - protein, 36 aa, unidentified
  source 1..36: note = Minute virus of mice
MAPPAKRAKRGWVPPGYKYLGPGNSLDQGEPTNPSD

SEQ ID NO: 164 - protein, 36 aa, unidentified
  source 1..36: note = Canine parvovirus
MAPPAKRARRGLVPPGYKYLGPGNSLDQGEPTNPSD

SEQ ID NO: 165 - protein, 60 aa, synthetic construct
  VARIANT 27: note = X can be any amino acid
MAPPAKRARRGKGVLVKWGEGKDLITXLSMCFFIGLVPPGYKYLGPGNSLDQGEPTNPSD

SEQ ID NO: 166 - protein, 54 aa, synthetic construct
LTVPGYKYLGPGNSLNRGQPINQIDEDAKEHDEAYDKVKTSQEVSRADNTFVNK

SEQ ID NO: 167 - protein, 54 aa, synthetic construct
LTVPGYKYLGPGNSLNRGQPTNQIDEDAKEHDEAYDKAKTSQEVSEADNTFVNK

SEQ ID NO: 168 - protein, 54 aa, synthetic construct
LTVPGYKYLGPGNSLNRGQPTNQIDEDAKEHDEAYDKAKTSQEVSQADNTFVNK

SEQ ID NO: 169 - protein, 54 aa, synthetic construct
LTVPGYKYLGPGNSLDRGEPVNQIDADAKEHDEAYDKAKTSQEVSDADSKFVSK

SEQ ID NO: 170 - protein, 54 aa, synthetic construct
LTVPGYKYLGPGNSLNRGPPTNEIDADAKEHDEAYSQSKTAQEVSKADNTFVNK

SEQ ID NO: 171 - protein, 54 aa, synthetic construct
LVPAPYKYAGPGNSLNRGPAYDLVDESARQHDIAYDKAKSPEDIHKADRQFLTE

SEQ ID NO: 172 - protein, 54 aa, synthetic construct
LTYPFHHYLGPGNPLDNNEPVDRDDAIAEEHDKAYANAKSSIDVINADKKAIDH

SEQ ID NO: 173 - protein, 57 aa, synthetic construct
AVLPGTDFVGPGNPIDPKPARSETDQIAKEHDLGYEDLLHRAKSQYFTEEDFKTEVY

SEQ ID NO: 174 - protein, 59 aa, synthetic construct
IHFPYHNYLGPGSDNFKKQPVDEDDAIARAHDLDYDKASSDKDIFKADKQARDEFFSSF

SEQ ID NO: 175 - protein, 59 aa, synthetic construct
IHFPYHNYLGPGTDNFEKNPVDEDDAIARSHDLAYDKVTNHKEVFQADKQARDEFFTSF

SEQ ID NO: 176 - protein, 59 aa, synthetic construct
LVPPGYKYLGPGNSLDQGEPTNPSDAAAKEHDEAYAAYLRSGKNPYLYFSPADQRFIDQ

SEQ ID NO: 177 - protein, 59 aa, synthetic construct
MVPPGYKYLGPGNSLDQGEPTNPSDAAAKEHDEAYDQYIKSGKNPYLYFSAADQRFIDQ

SEQ ID NO: 178 - protein, 59 aa, synthetic construct
WVPPGYKYLGPGNSLNQGEPTNPSDAAAKEHDEAYDQYIKSGKNPYLYFSPADQRFIDQ

SEQ ID NO: 179 - protein, 59 aa, synthetic construct
WVPPGYKYLGPGNSLDQGEPTNPSDAAAKEHDEAYDQYIKSGKNPYLYFSPADQRFIDQ

SEQ ID NO: 180 - protein, 59 aa, synthetic construct
CVPPGYKYLGPGNSLDQGEPTNPSDAAAKEHDLAYDEYIKSGKNPYLYFSPADQRFIDQ

SEQ ID NO: 181 - protein, 59 aa, synthetic construct
LTLPGYKYLGPGNSLDQGEPTNPSDAAAKEHDEAYDKYIKSGKNPYFYFSAADEKFIKE

SEQ ID NO: 182 - protein, 59 aa, synthetic construct
FVLPGYKYVGPGNGLDKGPPVNKADSVALEHDKAYDQQLKAGDNPYIKFKHADQEFIDN

SEQ ID NO: 183 - protein, 58 aa, synthetic construct
FVLPGYKYLPGNGLDKGPPVNKADSVALEHDKAYDQQLKAGDNPYIKFNHADQDFIDS

SEQ ID NO: 184 - protein, 59 aa, synthetic construct
LVLPGYKYLGPFNGLDKGEPVNEADAAALEHDKAYDRQLDSGDNPYLKYNHADAEFQER

SEQ ID NO: 185 - protein, 59 aa, synthetic construct
LVLPGYKYLGPGNGLDKGEPVNEADAAALEHDKAYDQQLKAGDNPYLKYNHADAEFQER

SEQ ID NO: 186 - protein, 59 aa, synthetic construct
LVLPGYKYLGPGNGLDKGEPVNAADAAALEHDKAYDQQLKAGDNPYLKYNHADAEFQQR

SEQ ID NO: 187 - protein, 59 aa, synthetic construct
LVLPGYKYLGPFNGLDKGEPVNAADAAALEHDKAYDQQLKAGDNPYLRYNHADAEFQER

SEQ ID NO: 188 - protein, 59 aa, synthetic construct
LTLPGYNYLGPFNSLFAGAPVNKADAAARKHDFGYSDLLKEGKNPYLYFNTHDQNLIDE

SEQ ID NO: 189 - protein, 59 aa, synthetic construct
LTLPFSNYIGPGNQLQAGNPQSVVDAAARIHDFRYSELIKLGINPYTHWSVADDELLHN

SEQ ID NO: 190 - protein, 59 aa, synthetic construct
LTLPLTHYIGPGNPLQAGSPTDVVDAAARIHDYRYSELIKLGINPYTHWTVADDELLHN

SEQ ID NO: 191 - protein, 59 aa, synthetic construct
IHLPADRYLGPGNPLENGPPVDPVDAVARIHDFRYADLEKQGINPYTTYTIADEELLKN

SEQ ID NO: 192 - protein, 59 aa, synthetic construct
VQLPGTNYVGPGNELQAGPPQSAVDSAARIHDFRYSQLAKLGINPYTHWTVADEELLKN

SEQ ID NO: 193 - protein, 47 aa, synthetic construct
DFADYGCYCGRGGSGTPVDDLDRCCQVHDNCYNEAAVCDCDRLAAIC

SEQ ID NO: 194 - protein, 47 aa, synthetic construct
EYNNYGCYCGLGGSGTPVDELDKCCQTHDNCYDQAFICNCDRNAAIC

SEQ ID NO: 195 - protein, 47 aa, synthetic construct
SYGFYGCHCGVGGRGSPKDATDRCCVTHDCCYKRLQLCECDKAAATC

SEQ ID NO: 196 - protein, 48 aa, synthetic construct
DYIYYGCYCGWGGKGKPIDATDRCCFVHDCCYGKMELCECEDRVAAIC

SEQ ID NO: 197 - protein, 47 aa, synthetic construct
SYYGYGCYCGLGGRGIPVDATDRCCWAHDCCYHKLKACECDKLSVYC

SEQ ID NO: 198 - protein, 52 aa, synthetic construct
IIYPGTLWCGHGNKSSGPNELGRFKHTDACCRTHDMCPDVMLSCDCDKFYDC

SEQ ID NO: 199 - protein, 47 aa, synthetic construct
NYGFYGCYCGWGGRGTPKDGTDWCCWAHDHCYGRLNLCACDRKLVYC

SEQ ID NO: 200 - protein, 47 aa, synthetic construct
AYMKYGCFCGLGGHGQPRDAIDWCCHGHDCCYTRALLCKCDQEIANC

SEQ ID NO: 201 - DNA, 180 bp, synthetic construct
  source 1..180: mol_type = other DNA
agaagattttcgagacgacttggattaaggtacgatggcacctccggcaaagagagccaggagaggtaagggtgtgttagtaaagtggggggaggggaaagatttaataacttaactaagtatgtgtttttttataggacttgtgcctccaggttataaatatcttgggcctgggaacag

SEQ ID NO: 202 - DNA, 180 bp, synthetic construct
  source 1..180: mol_type = other DNA
tcttctaaaagctctgctgaacctaattccatgctaccgtggaggccgtttctctcggtcctctccattcccacacaatcatttcacccccctcccctttctaaattattgaattgattcatacacaaaaaaatatcctgaacacggaggtccaatatttatagaacccggacccttgtc

SEQ ID NO: 203 - protein, 86 aa, synthetic construct
RRFSRRLGLRYDGTSGKESQERGCVSKVGEDFRDDLDGTMAPPAKRARRGKGVLVKWGKKIFETTWIKVRWHLRQREPGEVRVCSG

SEQ ID NO: 204 - protein, 79 aa, synthetic construct
GGERFNNLTKYVFFYRTCASRLISWAWEQEGKDLITLSMCFFIGLVPPGYKYLGPGNSLNVCVFLDLCLQVINILGLGT

SEQ ID NO: 205 - DNA, 180 bp, synthetic construct
  source 1..180: mol_type = other DNA
acttcagcgagccgctgaacttggactaaggtacgatggcgcctccagctaaaagagctaaaagaggtaagggtttaagggatggttggttggtggggtattaatgtttaattacctgttttacaggcctgaaatcacttggttttaggttgggtgcctcctggctacaagtacctggga

SEQ ID NO: 206 - DNA, 177 bp, synthetic construct
  source 1..177: mol_type = other DNA
tgaagtcgctcggcgacttgaacctgattccatgctaccgcggaggtcgattttctcgattttctccattcccaaattccctaccaaccaaccaccccataattacaaattaatggacaaaatgtccggactttagtgaaccaaaatccaacccacggaccgatgttcatggaccct

SEQ ID NO: 207 - protein, 84 aa, synthetic construct
TSASRTWTKVRWRLQLKELKEVRVGMVGLQRAAELGLRYDGASSKSKRGFKGWLVDFSEPLNLDGTMAPPAKRAKRGKGLRDGW

SEQ ID NO: 208 - protein, 80 aa, synthetic construct
WWGINVLPVLQANHLVLGWVPPGYKYLGGGVLMFNYLFYRPEITWFVGCLLATSTWDITCFTGLKSLGFRLGASWLQVPG

SEQ ID NO: 209 - DNA, 180 bp, synthetic construct
  source 1..180: mol_type = other DNA
acttcaacgaggagctgaccttggactaaggtacaatggcacctccagctaaaagagctaaaagaggtaaggggctaagggatggttggttggtggggtactaatgtatgactacctgttttacaggcctgaaatcacttggttctaggttgggtgcctcctggctacaagtacctggga

SEQ ID NO: 210 - DNA, 180 bp, synthetic construct
  source 1..180: mol_type = other DNA
tgaagttgctcctcgactggaacctgattccatgttaccgtggaggtcgattttctcgattttctccattccccgattccctaccaaccaaccaccccatgattacatactgatggacaaaatgtccggactttagtgaaccaagatccaacccacggaggaccgatgttcatggaccct

SEQ ID NO: 211 - protein, 84 aa, synthetic construct
TSTRSPWTKVQWHLQLKELKEVRGGMVGLQRGADLGLRYNGTSSKSKRGAKGWLVDFNEELTLDGTMAPPAKRAKRGKGLRDGW

SEQ ID NO: 212 - protein, 80 aa, synthetic construct
WWGTNVLPVLQANHLVLGWVPPGYKYLGGGVLMYDYLFYRPEITWFVGCLLATSTWDTTCFTGLKSLGSRLGASWLQVPG

SEQ ID NO: 213 - DNA, 270 bp, synthetic construct
  source 1..270: mol_type = other DNA
cctgaagaaacaactaacacagaagaactagctgcaacaggatatggccaaccactgatatccggaacggatgtgaatgccagctattagaaaagccagaggtaagtaaatatttcattatacactacacaatgcaaactacagaaaatcttctaaaagctctaccttgcttacttggatccatccaaccaatataggttgggtaccacctggatacaacttcctaggacccttcaatcaagacttcaacaaagaaccaactaatccatc

SEQ ID NO: 214 - DNA, 270 bp, synthetic construct
  source 1..270: mol_type = other DNA
ggacttctttgttgattgtgtcttcttgatcgacgttgtcctataccggttggtgactataggccttgcctacacttacggtcgataatcttttcggtctccattcatttataaagtaatatgtgatgtgttacgtttgatgtcttttagaagattttcgagatggaacgaatgaacctaggtaggttggttatatccaacccatggtggacctatgttgaaggatcctgggaagttagttctgaagttgtttcttggttgattaggtag

SEQ ID NO: 215 - protein, 84 aa, synthetic construct
PEETTNTEELAATGYGQPLISGTDVNASYLKKQLTQKNLQQDMANHYPERMMPAIRPRNNHRRTSCNRIWPTTDIRNGCECQLL

SEQ ID NO: 216 - protein, 87 aa, synthetic construct
KSQRVNISLYTTQCKLQKIFKLYLAYLDKARGKIFHYTLHNANYRKSSKSSTLLTWIEKPEVSKYFIIHYTMQTTENLLKALPCLLG

SEQ ID NO: 217 - protein, 87 aa, synthetic construct
PSNQYRLGTTWIQLPRTLQSRLQQRTNSIHPTNIGWVPPGYNFLGPFNQDFNKEPTNPSTIQPIVGYHLDTTSDPSIKTSTKNQLIH

SEQ ID NO: 218 - protein, 143 aa, synthetic construct
MAPPAKRARRGLVPPGYKYLGPGNSLDQGEPTNPSDAAAKEHDEAYAAYLRSGKNPYLYFSPADQRFIDQTKDAKDWGGKIGHYFFRAKKAIAPVLTDTPDHPSTSRPTKPTKRSKPPPHIFINLAKKKKAGAGQVKRDNLAP

SEQ ID NO: 219 - protein, 6 aa, synthetic construct
  VARIANT 1: note = K can be replaced by I
KRARRG

SEQ ID NO: 220 - protein, 4 aa, synthetic construct
LGPF

SEQ ID NO: 221 - DNA, 12 bp, synthetic construct
  source 1..12: mol_type = other DNA
tggttggttggt